(19) United States
(12) Patent Application Publication
(10) Pub. No.: US 2024/0166747 A1
(43) Pub. Date: May 23, 2024

(54) ANTIGEN BINDING PROTEINS AND COMBINATIONS THEREOF

(71) Applicant: GLAZOSMITHKLINE INTELLECTUAL PROPERTY DEVELOPMENT LIMITED, Stevenage (GB)

(72) Inventors: Yan Y. DEGENHARDT, Collegeville, PA (US); Jun GUAN, Collegeville, PA (US); Kenneth William HANCE, Collegeville, PA (US); Iris ROTH, South San Francisco, CA (US); Peter MORLEY, Stevenage (GB)

(21) Appl. No.: 18/552,477

(22) PCT Filed: Mar. 29, 2022
(86) PCT No.: PCT/IB2022/052897

§ 371 Date: Sep. 26, 2023

Related U.S. Application Data
(60) Provisional application No. 63/275,651, filed on Nov. 4, 2021.
(60) Provisional application No. 63/168,991, filed on Mar. 31, 2021.

(51) Int. Cl. C07K 16/28; A61P 35/00

(52) U.S. Cl. CPC C07K 16/2818; A61P 35/00; C07K 16/28; C07K 16/2803; C07K 2317/21; C07K 2317/24

(57) ABSTRACT

Provided are compositions for treating CD96 mediated diseases, and related methods.

### CROSS REFERENCE TO RELATED APPLICATIONS

This application claims priority from U.S. Provisional Application No. 63/168,991, filed 31 Mar. 2021, and U.S. Provisional Application No. 63/275,651, filed 4 Nov. 2021. The entire contents of each of the aforementioned applications are incorporated herein by reference.

### SEQUENCE LISTING SUBMITTED ELECTRONICALLY

The instant application contains a Sequence Listing which has been submitted electronically in ASCII format and is hereby incorporated by reference in its entirety. Said ASCII copy, created on 1 Feb. 2022, is named “PU67095_WO_SL_ASCII” and is 363 KB in size.

### FIELD OF THE INVENTION

The present invention relates to antigen binding proteins and fragments thereof that specifically bind to CD96 and in particular human CD96. The present invention also relates to methods of treating diseases or disorders with said antigen binding fragments, pharmaceutical compositions comprising said antigen binding fragments, and methods of manufacture. Other embodiments of the present invention will be apparent from the description below.

### BACKGROUND TO THE INVENTION

CD96/TACTILE (“T cell activation increased late expression”) is a cell surface receptor in the immunoglobulin superfamily, which is expressed mainly on T cells, natural killer (NK) cells, and natural killer T (NKT) cells. CD96 belongs to a family of receptors, which includes CD226 and TIGIT (“T cell immunoreceptor with Ig and ITIM domains,” also known as WUCAM, Vstm3, VSIG9) that are known to interact with nectin and nectin-like ligands. CD155/NECL5 (“nectin-like protein-5”) is the primary ligand for all three receptors (CD96, TIGIT and CD226). TIGIT binds to CD155 with higher affinity (3.15 nM) than CD226 (119 nM), and CD96 binding is intermediate (37.6 nM) (Martinet L. & Smyth M. J. Nat Rev Immunol. 2015 April; 15(4): 243-54). Besides CD155, both TIGIT and CD226 also bind another ligand CD112 with much reduced affinity. Recently a new receptor CD112R was discovered that also binds to CD112 (Zhu Y., et al. J Exp Med. 2016 Feb. 8; 213(2): 167-76)

Among the receptors in this axis, CD226 (DNAM-1) is one of the major activating receptors for NK cells. CD226 has been reported to potentiate NK cell cytotoxicity against cancer cells, and is critical for tumor immunosurveillance (Lakshmikanth T., et al. J Clin Invest. 2009; 119(5): 1251-63; Chan C. J., et al. J Immunol. 2010; 184(2): 902-11; Gilfillan S., et al. J Exp Med. 2008; 205(13): 2965-73; Iguchi-Manaka A., et al. J Exp Med. 2008; 205(13): 2959-64). Conversely, both CD96 (Chan C. J., et al. Nat Immunol. 2014; 15(5): 431-8) and TIGIT (Lozano E., et al. J Immunol. 2012; 188(8): 3869-75) are known to dampen immune responses through inhibition of NK and/or T cell function. TIGIT expression has been associated with T cell exhaustion (Lozano E., et al. 2012; Kurtulus S., et al. J Clin Invest. 2015; 125(11): 4053-62) and NK cell exhaustion (Zhang Q., et al. Nat Immunol. 2018; 19(7): 723-32), and several anti-TIGIT antibodies are in clinical development.

Overall, there is considerably more literature and mechanistic understanding on CD226 and TIGIT relative to CD96. CD226 does not have a classic ITAM motif as in other immune activating receptors. Upon ligand binding and receptor dimerization, it conducts a positive signaling through a series of phosphorylation events including PKC and Vav1 proteins. The cytoplasmic tail of TIGIT contains an ITT motif and a classic inhibitory ITIM motif. Upon CD155 binding, tyrosine phosphorylation of the ITT motif occurs, and immune inhibitory signaling is transduced downstream involving SHIP1. In contrast, no signaling for CD96 has yet been elucidated. It is known that there is a potentially inhibitory ITIM motif in the cytoplasmic tail of CD96, as well as a potentially activating YXXM motif that is present in multiple immune activating receptors (e.g. ICOS and CD28) (Georgiev H., et al. Front Immunol. 2018; 9: 1072).

Although CD96 was discovered over 25 years ago (Wang P. L., et al. J Immunol. 1992; 148(8): 2600-8), little was known about the function of CD96 other than the fact that it is a member of the immunoglobulin family that shares the ligand CD155 with CD226 and TIGIT (Fuchs A., et al. J Immunol. 2004; 172(7): 3994-8). Subsequent publications linking CD96 to cancer centered mostly on CD96 as a leukemia stem cell (LSC) marker. The first paper indicating CD96 as a potential immuno oncology target was published by the lab of Professor Mark Smyth in 2014; CD96 was shown to compete with CD226 for CD155 binding in NK cells and CD96 negatively regulated production of pro-inflammatory cytokines including IFNγ (gamma) in mice after activation by LPS (Chan C. J., et al. 2014). CD96 knockout mice as well as anti-CD96 antibody-treated mice were less susceptible to MCA-induced sarcoma formation (Id.). In the same study, CD226 or CD155 blockade resulted in a worse outcome, and this is postulated to be due to the loss of the CD155:CD226 activatory pathway (Id.).

Subsequently, further in vivo studies supporting the inhibition of CD96 for cancer treatment have been published (See, e.g. Blake S. J., et al. Cancer Discov. 2016; 6(4): 446-59; Brooks J., et al. 2018; 78(2): 475-88; Harjunpää H., et al. Oncoimmunology. 2018; 7(7): e1445949). Accordingly, a need exists for improved antigen binding proteins and fragments thereof that target CD96 for use in the treatment of disease. Such compositions and related methods are provided in the present disclosure.

### SUMMARY OF THE INVENTION

The present invention provides, in a first aspect, CD96 binding proteins. The present invention also provides, in a second aspect, nucleic acid constructs encoding CD96 binding proteins. In a third aspect, the present invention provides expression vectors comprising the nucleic acid according to the second aspect. In a fourth aspect, the present invention provides a recombinant host cell comprising the nucleic acid or expression vector described in the previous aspects. The present invention further provides, in a fifth aspect, methods of producing a CD96 binding protein comprising culturing the host cell as described in the preceding aspect under conditions suitable for expression of said nucleic acid sequence(s) or vector(s), whereby a polypeptide comprising the CD96 binding protein is produced. A sixth aspect of the disclosure is the CD96 binding protein produced by the method for the production described in the preceding aspect. The present invention also provides, in a seventh aspect, pharmaceutical compositions comprising the CD96 binding protein described in any one of the preceding aspects, and a pharmaceutically acceptable excipient. Another aspect of the disclosure is a method of treatment of a disease in a subject in need thereof comprising administering to said subject a therapeutically effective amount of the CD96 binding protein or the pharmaceutical composition as described in any one of the preceding aspects to the subject. A further aspect of the disclosure is the method of treatment described in the preceding aspect further comprising whether the subject expresses CD96. Another aspect of the disclosure is a CD96 binding protein or a pharmaceutical composition as described in any one of the preceding aspects for use in therapy or for use in the treatment of a disease.

The following detailed description of preferred embodiments of the invention will be better understood when read in conjunction with the appended drawings. For the purpose of illustrating the invention, there are shown in the drawings, embodiments which are presently preferred. It should be understood, however, that the invention is not limited to the precise arrangements and instrumentalities of the embodiments shown in the drawings.

### DESCRIPTION OF DRAWINGS/FIGURES

FIG. 1 shows binding of CD96 binding proteins to human CD3⁺ T cells (A), and the ability for CD96 binding proteins to prevent the binding of CD155 to CHO cells expressing human CD96 (B).

FIG. 2 shows solution equilibrium titration (SET) data for CD96 binding protein binding to human (a), cynomolgus monkey (b) or mouse (c) CD96.

FIG. 3 shows binding of CD96 binding protein to HEK cells transiently transfected with human or cynomolgus monkey CD96 isoforms.

FIG. 4 shows binding of CD96 binding protein (vs isotype control) to primary human T cells (total CD3⁺ T cells vs CD4⁺ or CD8⁺ subsets).

FIG. 5 shows binding of CD96 binding protein to activated primary cynomolgus monkey T cells.

FIG. 6 shows CD96 internalisation using imaging cytometry in CD8⁺ T cells following binding to human PBMCs using imaging cytometry; representative cell images of CD8⁺ T cells (Gray=CD8 staining, white=CD96 binding protein-PE staining)

FIG. 7 shows CD96 binding protein, when pre-complexed to membrane CD96 in primary human T cells, preventing the binding of CD155:Fc to said T cells.

FIG. 8 shows the displacement of CD155:Fc that has been pre-bound to human T cells by CD96 binding protein.

FIG. 9 shows human Fcγ reporter assay data, demonstrating that binding of CD96 binding proteins to CD96 on primary human T cells does not induce cross-linking and/or signaling via Fcγ receptors.

FIG. 10 shows human ADCC target cell killing assay data in CD4+ and CD8⁺ T cells; no evidence of increased cell death was observed in either CD4+ or CD8+ T cells in the presence of CD96 binding proteins.

FIG. 11 shows human CDC target cell killing assay in CD4⁺ T cells; no evidence of induced complement-dependent cellular toxicity is observed from CD96 binding protein.

FIG. 12 shows the EC50 data for IFNγ release of CD96 binding proteins (expressed from CHO or HEK cells) in a mixed PMBC-MLR assay.

FIG. 13 shows activity of CD96 binding protein (expressed from HEK cells) on IFNγ production in a mixed PBMC-MLR assay, with or without CD4+(A) or CD8+(B) T cells.

FIG. 14 shows the effect of CD96 binding protein on the secretion of IFNγ and Granzyme B in a mixed PBMC-MLR assay.

FIG. 15 shows the frequency of IFNγ+ cells in different cell populations on day 3 in a mixed PBMC-MLR assay, in the presence of CD96 binding protein and controls.

FIG. 16 shows the frequency of CD96⁺ cells in CD4⁺, CD8⁺, and NK cell populations upon treatment with CD96 binding protein and controls in a mixed PBMC-MLR assay.

FIG. 17 shows expression level of CD96 in CD4⁺, CD8⁺, and NK cell populations upon treatment with CD96 binding proteins and controls in a mixed PBMC-MLR assay.

FIG. 18 shows FACs characterization displaying the effect of CD96 binding protein on the ratio of CD226⁺ single positive vs CD226⁺CD96⁺ double positive NK cells in a mixed PBMC-MLR assay.

FIG. 19 shows FACs characterization displaying the effect of CD96 binding protein on IFNγ⁺GrzB⁺ double positive cells among NK cell total population in a mixed PBMC-MLR assay.

FIG. 20 shows the inhibitory effect of plate-bound CD155-Fc on IFNγ production in a human PBMC assay.

FIG. 21 shows the activity of CD96 binding protein in renal cancer TIL functional assays, alone or in combination with anti-PD1 or anti-TIGIT antibodies.

FIG. 22 shows bioluminescence imaging study data of CD96 binding protein in a NK cell dependent B16F10 melanoma lung colonization model, showing representative bioluminescent images acquired approximately 15 minutes post injection of B16F10 RFluc melanoma cells.

FIG. 23 shows lung bioluminescent signal at Day 14 post B16F10 cell injection in mice without depletion; with CD4, CD8, or NK cell depletion.

FIG. 24 shows in vivo bioluminescent lung signals at day 14 and day 20 exhibiting the effect of CD96 binding proteins (vs control) on lung metastasis in various groups.

FIG. 25 shows images of CD96 binding protein treated CD4⁺/CD8⁺ depleted mouse lungs vs control at day 20 (end of study).

FIG. 26 shows IFNγ production in CD155 coated PBMC assay in the presence of CD96 binding proteins or isotype control in the presence of anti-TIGIT mAb, as evaluated by MSD.

FIG. 27 shows TNFα production in CD155 coated PBMC assay in the presence of CD96 binding proteins or isotype control in the presence of anti-TIGIT mAb, as evaluated by MSD.

FIG. 28 shows IFNγ production in CD155 coated PBMC assay in the presence of CD96 binding proteins or isotype control in the presence or absence of anti-TIGIT mAb for 3 days, as evaluated by MSD.

FIGS. 29A-29D show CD226 axis expression across immune subsets in human tumors, determined by CyTOF as described in Example 11. FIG. 29A: Live CD45+ cells from a subset of donors (61 were concatenated and analyzed via uniform manifold approximation and projection (UMAP) analysis). Expression of canonical immune markers were used to gate N=12 cell subsets and define biaxial gating strategies. FIG. 29B: Frequency of biaxially gated populations amongst samples across N=14 tumor indications and all samples (n=104). FIG. 29C: UMAPs highlighting CD226 axis member expression, color coded for signal intensity (median mass intensity, MMI). FIG. 29D: Collated percent (%) expression of CD226 axis members across major immune cell subsets. Each symbol represents a single sample.

FIGS. 30A-30D show CD226 axis expression on NK cell subsets across tumor indications, determined by CyTOF as described in Example 11. FIG. 30A: NK cells from all donors with a minimum of 30 NK cells (76 as identified by biaxial gating) were concatenated for UMAP analysis The biaxial gating definition for NK cells (as informed by FIG. 29A UMAP analysis) included CD56+ and/or NKG2D⁺ cells that were also CD3⁻ CD19⁻ CD15⁻ CD11b⁻ CD11c⁻. Regions of interest (ROIs) with unique protein expression patterns are shown in FIGS. 30A-30B. FIG. 30B: NK cell UMAPs highlighting CD226 axis member expression, color coded by signal intensity (MMI). FIG. 30C: Frequency of NK cells that fell within each ROI (as gated on UMAP) amongst each sample across tumor indications. FIG. 30D: Signal intensity (median metal intensity, MMI) of various markers amongst NK ROIs. Various clusters are highlighted (boxed). Markers with MMIs of <5 across all ROIs and CD226 axis ligands (CD155 and CD112) were removed. PVRIG based analysis was conducted on N=61 samples.

FIGS. 31A-31D show CD226 axis expression on CD4+ T cell subsets across human tumors, determined by CyTOF as described in Example 11. FIG. 31A: CD4+ T cells from all donors with a minimum of 30 CD4+ T cells (n=97) as identified by biaxial gating were concatenated for UMAP analysis. The biaxial gating definition for tumor-infiltrating CD4+ T cells included CD3⁺ CD4⁺ cells that were also CD8⁻ CD19⁻ CD56⁻ CD15⁻ CD14⁻ CD11b⁻ with FoxP3 expression defining Treg cells. On the resulting CD4⁺ T cell UMAP, regions of interest (ROIs) with unique protein expression patterns are shown in FIG. 31A and FIG. 31B. FIG. 31B: CD4⁺ T cell UMAPs highlighting CD226 axis member expression, color-coded by signal intensity (MMI). FIG. 31C: Frequency of CD4⁺ T cells that fell within each ROI (as gated on UMAP) amongst samples across tumor indications. FIG. 31D: Signal intensity (MMI) of various markers amongst CD4⁺ T cell ROIs. Various clusters are highlighted (boxed). Markers with minimal expression (MMIs of <5 across all ROIs) and CD226 axis ligands (CD155 and CD112) were removed. PVRIG based analysis was conducted on N=61 samples.

FIGS. 32A-32D show CD226 axis expression on CD8+ T cell subsets across human tumors, determined by CyTOF as described in Example 11. FIG. 32A: CD8+ T cells from all donors with a minimum of 30 CD8+ T cells (n=90) as identified by biaxial gating were concatenated for UMAP analysis. The biaxial gating definition for tumor-infiltrating CD8+ T cells included CD3+CD+8 cells that were also CD4− CD19−CD56−CD15−CD14−CD11b−. On the resulting CD8+ T cell UMAP, regions of interest (ROIs) with unique protein expression patterns are shown in FIGS. 32A-32B. FIG. 32B: CD8+ T cell UMAPs highlighting CD226 axis member expression, color coded by signal intensity (MMI). FIG. 32C: Frequency of CD8+ T cells that fell within each ROI (as gated on UMAP) amongst each sample across tumor indications. FIG. 32D: Signal intensity (MMI) of various markers amongst CD8+ T cell ROIs. Various clusters are highlighted (boxed). Markers with minimal expression (MMIs of less than 5 across all ROIs) and CD226 axis ligands (CD155 and CD112) were removed. PVRIG based analysis was conducted on N=61 samples.

FIG. 33 shows a summary of CD226 axis member (CD96, PVRIG, TIGIT, CD226) and PD-1 expression on human tumor infiltrating lymphocytes (TILs), determined by CyTOF as described in Example 11. Expression level is signified by − (no expression) to ++++ (very high) relative expression. Relative expression compares total overall expression across all relevant immune subsets.

### DETAILED DESCRIPTION OF THE INVENTION

The present disclosure provides CD96 binding proteins, nucleic acids encoding said proteins, and related subject matter.

As used herein and in the claims, the singular forms “a,” “and,” and “the” include plural reference unless the context clearly dictates otherwise. Thus, for example, reference to “a peptide chain” is a reference to one or more peptide chains and includes equivalents thereof known to those skilled in the art.

As used herein and in the claims, the term “comprising” encompasses “including” or “consisting” e.g. a composition “comprising” X may consist exclusively of X or may include something additional, e.g., X+Y.

The term “consisting essentially of” limits the scope of the feature to the specified materials or steps and those that do not materially affect the basic characteristic(s) of the claimed feature.

The term “consisting of” excludes the presence of any additional component(s).

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range. The figures and tables of the disclosure also describe ranges, and discrete values, which may constitute an element of any of the methods disclosed herein.

Concentrations described herein are determined at ambient temperature and pressure. This may be, for example, the temperature and pressure at room temperature or in within a particular portion of a process stream. Preferably, concentrations are determined at a standard state of 25° C. and 1 bar of pressure.

The term “about” means a value within two standard deviations of the mean for any particular measured value.

“Affinity” is the strength of binding of one molecule to another. The binding affinity of an antigen binding protein to its target may be determined by equilibrium methods (e.g. enzyme-linked immunosorbent assay (ELISA) or radioimmunoassay (RIA)), or kinetics (e.g. surface plasmon resonance analysis using e.g. BIACORE equipment).

The term “antigen” as used herein refers to a structure of a macromolecule which is selectively recognized by an antigen binding protein. Antigens include but are not limited to protein (with or without polysaccharides) or protein composition comprising one or more T cell epitopes. As is contemplated herein, the target binding domains an antigen binding protein may recognize a sugar side chain of a glycoprotein rather than a specific amino acid sequence or of a macromolecule. Thus, the sugar moiety or sulfated sugar moiety serves as an antigen.

The term “antigen binding protein”, as used herein refers to isolated proteins, antibodies, antibody fragments (e.g., Fabs etc.) and other antibody derived protein constructs, such as those comprising domains (e.g., domain antibodies etc.) which are capable of binding to CD96. Such alternative antibody formats include triabody, tetrabody, miniantibody, and a minibody. Also included are alternative scaffolds in which the one or more CDRs of any molecules in accordance with the disclosure can be arranged onto a suitable non-immunoglobulin protein scaffold or skeleton, such as an affibody, a SpA scaffold, an LDL receptor class A domain, an avimer (see, e.g., U.S. Patent Application Publication Nos. 2005/0053973, 2005/0089932, 2005/0164301) or an EGF domain. An ABP also includes antigen binding fragments of such antibodies or other molecules. Further, an ABP may comprise the VH regions of the invention formatted into a full length antibody, a (Fab′)2 fragment, a Fab fragment, a bi-specific or biparatopic molecule or equivalent thereof (such as scFV, bi- tri- or tetra-bodies, Tandabs, etc.), when paired with an appropriate light chain. The ABP may comprise an antibody that is an IgG1, IgG2, IgG3, or IgG4; or IgM; IgA, IgE or IgD or a modified variant thereof. The constant domain of the antibody heavy chain may be selected accordingly. The light chain constant domain may be a kappa or lambda constant domain. The ABP may also be a chimeric antibody of the type described in WO86/01533, which comprises an antigen binding region and a non-immunoglobulin region. The antigen binding proteins of the disclosure can be provided as a lyophilized powder containing the antibody and excipients which can be reconstituted with a pharmaceutically acceptable carrier (e.g., sterile water). This reconstituted pharmaceutical composition can then be administered either subcutaneously or intravenously (e.g., with further dilution). The antigen binding proteins of the disclosure can also be provided as a liquid formulation containing the antibody, excipients and a pharmaceutically acceptable carrier. This liquid pharmaceutical composition can then be administered either subcutaneously or intravenously (e.g., with further dilution). The terms “ABP,” “antigen binding protein,” and “binding protein” are used interchangeably herein. As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “antibody variant” as used herein means an antibody that differs from a parent antibody by virtue of at least one amino acid modification (e.g., by having a different amino acid side chain), post-translational modification or other modification in at least one heavy chain, light chain, or combinations of these that results in a structural change (e.g., different amino acid side chain, different post-translational modification or other modification) relative to the parent antibody. Structural changes can be determined directly by a variety of methods well known in the art such as LC-MS, direct sequencing or indirectly via methods such as isoelectric focusing and the like. Such methods are well known to those of ordinary skill in the art.

The term “epitope” as used herein refers to that portion of the antigen that makes contact with a particular binding domain. An epitope may be linear or conformational/discontinuous. A conformational or discontinuous epitope comprises amino acid residues that are separated by other sequences, i.e. not in a continuous sequence in the antigen's primary sequence. Although the residues may be from different regions of the peptide chain, they are in close proximity in the three dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography. As is contemplated herein the term epitope includes post-translational modification to a polypeptide that can be recognized by an antigen binding protein or domain, such as sugar moiety of a glycosylated protein.

The term “isolated” as used herein, means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

CD96 (Cluster of Differentiation 96), also known as TACTILE, is a receptor expressed on T cells and NK cells, shares sequence similarity with CD226 (DNAM01), and is a type 1 transmembrane glycoprotein belonging to the immunoglobulin superfamily.

Human CD96 has 3 isoforms: two membrane (V1; V2) and a soluble form. The longer V1 isoform is a 585 amino acid protein with a MW of 65,634 Da. A shorter isoform V2 (569 aa) has a MW of 63,888 Da. V2 differs from V1 by having a short deletion of the Ig fold of the second domain. The first domain of CD96 is reported to contain the epitope(s) required for CD155 binding while the second domain modulates the magnitude/strength of binding. CD96V2 binds much more strongly to CD155 than does CD96V1, and is also a predominantly expressed form with the exception of acute myeloid leukemia AML (cells). Little is known about the soluble form of CD96 (sCD96). It has been reported that sCD96 was detected in healthy donor blood (1-3 ng/ml), with higher levels detected in hepatitis B virus (HBV) HBV/liver cirrhosis patients.

Based on mRNA analysis of normal human tissues the highest expression of CD96 (probe detecting both V1 and V2 of CD96) is observed in hematopoietic cells. CD96 expression is also observed in tissues containing large numbers of lymphocytes, such as spleen, lungs, thyroid and small intestine. Among hematopoietic cells, CD96 is most abundant in T cells and NK cells, with lower expression in some B cells. Like CD96, TIGIT and CD226 are most abundant in hematopoietic cells, and expressed in both T cells and NK cells. CD226 is also expressed in conventional DC cells. The ligand CD155 shows a broad expression pattern in human normal tissue and is also expressed in DC cells and macrophages (antigen presenting cells). CD155 is not expressed in T cells or NK cells.

As used herein, “CD96 antagonist” or “CD96 binding protein” means any chemical compound or biological molecule that blocks binding of CD96 expressed on a T cell or NK cell to CD155.

The binding affinity (KD) of the antigen binding protein-target antigen interaction may be 1 mM or less, 100 nM or less, 10 nM or less, 2 nM or less or 1 nM or less. Alternatively, the KD may be between 5 and 10 nM; or between 1 and 2 nM. The KD may be between 1 pM and 500 pM; or between 500 pM and 1 nM. For example certain useful such variants have a binding affinity (KD) which is at least about 40 nM or at least about 35 nM or at least about 30 nM e.g. about 10 pM to about 30 nM.

The binding affinity of the antigen binding protein is determined by the association constant (Ka) and the dissociation constant (Kd) (KD=Kd/Ka). The binding affinity may be measured by surface plasmon resonance (e.g. using BIACORE equipment), for example, by capture of the test antibody onto a protein-A coated sensor surface and flowing target antigen over this surface. Alternatively, the binding affinity can be measured by FORTEBIO, for example, with the test antibody receptor captured onto a protein-A coated needle and flowing target antigen over this surface. Alternatively the binding affinity (KD) can be measured by using MSD-SET analysis (MSD solution equilibrium titration) for example with the test antibody titrarated onto a standard bind MSD plate and detected using an MSD SECTOR IMAGER. MSD-SET determines the solution phase, equilibrium affinity of antibodies. This known method relies on the detection of free antigen at equilibrium in a titrated series of antibody concentrations.

The Kd may be 1×10−3 Ms−1 or less, 1×10−4 Ms−1 or less, or 1×10−5 Ms−1 or less. The Kd may be between 1×10−5 Ms−1 and 1×10−4 Ms−1; or between 1×10−4 Ms−1 and 1×10−3 Ms−1. A slow Kd may result in a slow dissociation of the antigen binding protein-target antigen complex and improved neutralization of the target antigen.

The term “specific antigen binding activity” as used herein means antigen binding activity as measured e.g. by Surface Plasmon Resonance (SPR). CD96 specific binding activity may be determined by SPR using a BIACORE instrument, for example performed in the binding mode. It is binding activity divided by total protein content in a sample.

The terms “VH” and “VL” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least one CDR and wherein the at least one CDR is CDRH3. Framework regions follow each of these CDR regions. Acceptable heavy chain variable region and light chain variable region framework 1, framework 2 and framework 3 regions are readily recognized by those of ordinary skill in the art. Acceptable heavy chain constant regions (including hinge regions) and light chain constant regions are readily recognized by those of ordinary skill in the art as well. Acceptable antibody isotypes are similarly readily recognized by those of ordinary skill in the art.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the specification follow the Kabat numbering convention.

It will be apparent to those skilled in the art that there are alternative numbering conventions for amino acid residues in variable domain sequences and full length antibody sequences. There are also alternative numbering conventions for CDR sequences, for example those set out according to the Chothia numbering convention. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

Table 1 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 1

| | | | | | Minimum |
|---|---|---|---|---|---|
| | Kabat CDR | Chothia CDR | AbM CDR | Contact CDR | binding unit |
| H1 | 31-35/35A/35B | 26-32/33/34 | 26-35/35A/35B | 30-35/35A/35B | 31-32 |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

In one embodiment of the disclosure is a CD96 binding protein comprising: (a) (i) any one or a combination of CDRs selected from CDRH1, CDRH2, CDRH3 from SEQ ID NOS: 2, 6, 10, 14, 18, 22, 26, 30, 34, 38, 42, 46, 50, 54, 58, 62, 66, 70, 74, 78, 82, 86, 90, and 94, and/or CDRL1, CDRL2, CRDL3 from SEQ ID NOS: 1, 5, 9, 13, 17, 21, 25, 29, 33, 37, 41, 45, 49, 53, 57, 61, 65, 69, 73, 77, 81, 85, 89, and 93; or (ii) a CDR variant of (i) wherein the variant has 1, 2, or 3 amino acid modifications; or (b) a VH region comprising a sequence at east 80% identical (or at least 85%, 90%, 91%, 92%, 93%, 94%, 95%, 96%, 97%, 98%, 99%, 99.5% identical) to the sequence of SEQ ID NOS: 2, 6, 10, 14, 18, 22, 26, 30, 34, 38, 42, 46, 50, 54, 58, 62, 66, 70, 74, 78, 82, 86, 90, or 94; and/or a VL region comprising a sequence at least 80% identical to the sequence of SEQ ID NOS: 1, 5, 9, 13, 17, 21, 25, 29, 33, 37, 41, 45, 49, 53, 57, 61, 65, 69, 73, 77, 81, 85, 89, or 93.

In another embodiment, the CD96 binding protein comprises: (a) (i) any one or a combination of CDRs selected from CDRH1, CDRH2, CDRH3 from SEQ ID NOS: 2, 6, 10, 14, 18, 22, 26, 30, 34, 38, 42, 46, 50, 54, 58, 62, 66, 70, 74, 78, 82, 86, 90, and 94, and/or CDRL1, CDRL2, CRDL3 from SEQ ID NOS: 1, 5, 9, 13, 17, 21, 25, 29, 33, 37, 41, 45, 49, 53, 57, 61, 65, 69, 73, 77, 81, 85, 89, and 93; or (ii) a CDR variant of (i) wherein the variant has 1, 2, or 3 amino acid modifications; or (b) a VH region comprising a sequence of SEQ ID NOS: 2, 6, 10, 14, 18, 22, 26, 30, 34, 38, 42, 46, 50, 54, 58, 62, 66, 70, 74, 78, 82, 86, 90, or 94; and/or a VL region comprising a sequence at least 80% identical (or at least 85%, 90%, 91%, 92%, 93%, 94%, 95%, 96%, 97%, 98%, 99%, 99.5% identical) to the sequence of SEQ ID NO: 1, 5, 9, 13, 17, 21, 25, 29, 33, 37, 41, 45, 49, 53, 57, 61, 65, 69, 73, 77, 81, 85, 89, or 93.

Another embodiment of the disclosure is the CD96 binding protein described in the previous aspect comprising a CDRH1 selected from SEQ ID NOS: 105-125; a CDRH2 selected from SEQ ID NOS: 126-146; and/or a CDRH3 selected from SEQ ID NOS: 147-150; a CDRL1 selected from of SEQ ID NOS: 97-98; a CDRL2 selected from SEQ ID NOS: 99-100; and/or a CDRL3 selected from SEQ ID NOS: 101-104.

Another embodiment of the disclosure is the CD96 binding protein described in any one of the preceding aspects wherein the binding protein comprises CDRH3 that is 100% identical to Seq ID NOS: 147, 148, 149, or 150.

Another embodiment of the disclosure is the CD96 binding protein described in any one of the preceding aspects comprising a CDRH1 that is 100% identical to SEQ ID NO: 105, 106, 107, 108, 109, 110, 111, 112, 113, 114, 115, 116, 117, 118, 119, 120, 121, 122, 123, 124 or 125; a CDRH2 that is 100% identical to SEQ ID NO: 126, 127, 128, 129, 130, 131, 132, 133, 134, 135, 136, 137, 138, 139, 140, 141, 142, 143, 144, 145, or 146; and/or a CDRH3 that is 100% identical to SEQ ID NO: 147, 148, 149, or 150; a CDRL1 that is 100% identical to SEQ ID NO: 97 or 98; a CDRL2 that is 100% identical to SEQ ID NO: 99 or 100; and/or a CDRL3 that is 100% identical to SEQ ID NO: 101, 102, 103, or 104.

Another embodiment of the disclosure is the CD96 binding protein described in any one of the preceding aspects wherein all 6 CDRs are present in the binding protein.

Another embodiment of the disclosure is the CD96 binding protein described in any one of the preceding aspects comprising a CDRH1 of SEQ ID NO: 115; a CDRH2 of SEQ ID NO: 145; and a CDRH3 of SEQ ID NO: 147; and/or a CDRL1 of SEQ ID NO: 97; a CDRL2 of SEQ ID NO: 99; and a CDRL3 of SEQ ID NO: 101.

Another embodiment of the disclosure is the CD96 binding protein described in any one of the preceding aspects wherein the binding protein comprises: a VH region that is 75% identical to SEQ ID NO: 86; and/or a VL region that is 75% identical to SEQ ID NO: 85 (or at least 85%, 90%, 91%, 92%, 93%, 94%, 95%, 96%, 97%, 98%, 99%, 99.5% identical to these sequences). Another aspect of the disclosure is the CD96 binding protein described in any one of the preceding aspects wherein the binding protein comprises: a VH region that is 100% identical to SEQ ID NO: 86; and/or a VL region that is 100% identical to SEQ ID NO: 85.

In one embodiment of the disclosure the invention provides a CD96 binding protein which comprises the any of the CDRs as described herein (alone or in the combinations described) and which also comprise an Fc region which can bind to the Fc gamma receptor and/or can promote IFNgamma release. Such an Fc region can be the wild type IgG1Fc.

In one embodiment of the disclosure the invention provides a CD96 binding protein which comprises the any of the VH regions as described herein (alone or in the combinations described) and which also comprise an Fc region which can bind to the Fc gamma receptor and/or can promote IFNgamma release. Such an Fc region can be the wild type IgG1Fc.

In one embodiment of the disclosure the invention provides a CD96 binding protein which comprises the any of the VL regions as described herein (alone or in the combinations described) and which also comprise an Fc region which can bind to the Fc gamma receptor and/or can promote IFNgamma release. Such an Fc region can be the wild type IgG1Fc.

Another embodiment of the disclosure is the CD96 binding protein as described in any one of the preceding aspects, which is an antibody, wherein the binding protein comprises: a (complete) heavy chain that is 75% identical (or at least 80%, 85%, 90%, 91%, 92%, 93%, 94%, 95%, 96%, 97%, 98%, 99%, 99.5% identical) to SEQ ID NO: 165; and/or a (complete) light chain that is 75% identical (or at least 80%, 85%, 90%, 91%, 92%, 93%, 94%, 95%, 96%, 97%, 98%, 99%, 99.5% identical) to SEQ ID NO: 166.

Another aspect of the disclosure is the CD96 binding protein described in any one of the preceding aspects wherein the binding protein comprises: a VH region that is 100% identical to SEQ ID NO: 86; and/or a VL region that is 100% identical to SEQ ID NO: 85. Another aspect of the disclosure is the CD96 binding protein described in any one of the preceding aspects, which is an antibody, and wherein the binding protein comprises: a (complete) heavy chain that is 100% identical to the amino acid sequence of SEQ ID NO: 165; and/or a (complete) light chain that is 100% identical to the amino acid sequence of SEQ ID NO: 166 (CDRs are underlined in SEQ ID Nos 165 and 166); or is an antibody which binds to CD96 and wherein the heavy chain is encoded by the nucleic acid sequence of SEQ ID NO: 167 and/or the light chain is encoded by the nucleic acid sequence of SEQ ID NO: 168.

Another aspect of the disclosure is the CD96 binding protein described in any one of the preceding aspects, which is an antibody, and wherein the binding protein comprises: a (complete) heavy chain that is 100% identical to the amino acid sequence of SEQ ID NO: 170; and/or a (complete) light chain that is 100% identical to the amino acid sequence of SEQ ID NO: 169 (CDRs are underlined) or is an antibody which binds to CD96 and wherein the heavy chain is encoded by the nucleic acid sequence of SEQ ID NO: 172 and/or the light chain is encoded by the nucleic acid sequence of SEQ ID NO: 171.

Another aspect of the disclosure is the CD96 binding protein described in any one of the preceding aspects, which is an antibody, and wherein the binding protein comprises: a (complete) heavy chain that is 100% identical to the amino acid sequence of SEQ ID NO: 174; and/or a (complete) light chain that is 100% identical to the amino acid sequence of SEQ ID NO: 173 (CDRs are underlined) or is an antibody which binds to CD96 and wherein the heavy chain is encoded by the nucleic acid sequence of SEQ ID NO: 176 and/or the light chain is encoded by the nucleic acid sequence of SEQ ID NO: 175.

The invention includes binding proteins which are 100% identical to any of the amino acid sequences described herein and also proteins which are variants of the amino acid sequences described herein e.g. sequences which are at least 75% identical or at least 80%, 85%, 90%, 91%, 92%, 93%, 94%, 95%, 96%, 97%, 98%, 99%, 99.5%, identical to the sequences herein.

The invention also included nucleic acids encoding the binding proteins of the invention including nucleic acids which are 100% identical to any of the nucleic acid sequences described herein and also nucleic acids which are variants of the sequences described herein e.g. sequences which are at least 75% identical or at least 80%, 85%, 90%, 91%, 92%, 93%, 94%, 95%, 96%, 97%, 98%, 99%, 99.5%, identical to the sequences herein.

CDRs and variable regions with amino acid sequences provided in the present application have binding affinity to CD96 which is equal to or better than that of CD155 and/or are variable regions which can prevent or displace CD155 from binding to CD96 (examples of binding assays which can be used to determine this are set out in examples 1 and 2 herein). Useful variants of the sequences described herein are variants in which the variable regions have binding affinity to CD96 which is equal to or better than that of CD155 or which can prevent or displace CD155 from binding to CD96 (examples of binding assays which can be used to determine this are set out in examples 1 and 2 herein). For example certain useful such variants have a binding affinity (KD) which is at least about 40 nM or at least about 35 nM or at least about 30 nM e.g. about 10 pM to about 30 nM when for example binding affinity is determined by MSD-SET assays as detailed herein.

Useful CD96 binding proteins (such as antibodies) according to the present invention can comprise such variable regions and also an Fc region as described herein wherein said Fc region can bind to the Fc gamma receptor and/or can promote IFN gamma release. Such an Fc region according to the invention is the wild type IgG1Fc or a functional variant thereof (for example an Fc disabled variant which region can bind to the Fc gamma receptor and/or can promote IFN gamma release). Hence any of the CDRs and/or variable regions of the invention can be combined with an Fc region of the invention e.g. the wild type IgG1 Fc or a functional variant thereof.

Another embodiment of the disclosure is the CD96 binding protein described in any one of the preceding aspects wherein the binding protein comprises a synthetic polypeptide, a humanised sequence, or a chimeric sequence.

As used herein, the term “conservative sequence modifications” is intended to refer to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “domain” refers to a folded protein structure which retains its tertiary structure independent of the rest of the protein. Generally, domains are responsible for discrete functional properties of proteins and in many cases, may be added, removed or transferred to other proteins without loss of function of the remainder of the protein and/or of the domain.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In one embodiment of the disclosure is a nucleic acid sequence which encodes the CD96 binding protein described in any one of the preceding embodiments.

A further embodiment of the disclosure is the nucleic acid sequence described in the previous embodiment wherein the sequence comprises SEQ ID NO 4, 8, 12, 16, 20, 24, 28, 32, 36, 40, 44, 48, 52, 56, 60, 64, 68, 72, 76, 80, 84, 88, 92, or 96 encoding the heavy chain; and/or SEQ ID NO: 3, 7, 11, 15, 19, 23, 27, 31, 35, 39, 43, 47, 51, 55, 59, 63, 67, 71, 75, 79, 83, 87, 91, or 95 encoding the light chain. Another embodiment of the disclosure is an expression vector comprising the nucleic acid described in any of the preceding embodiments.

The compositions described herein may be produced by any number of conventional techniques. For example, the compositions may be expressed in and purified from recombinant expression systems. In one embodiment, the composition is produced by a method of culturing a host cell under conditions suitable for expression of CD96 binding protein described in any of the preceding embodiments wherein the composition is expressed, and optionally purified, and optionally formulated within a pharmaceutical composition.

A number of different expression systems and purification regimes can be used to produce the compositions. Generally, host cells are transformed with a recombinant expression vector encoding the antibody. A wide range of host cells can be employed, including Eukaryotic cell lines of mammalian origin (e.g., CHO, Perc6, HEK293, HeLa, NS0). Suitable host cells include mammalian cells such as CHO (e.g., CHOK1 and CHO-DG44).

The host cell may be an isolated host cell. The host cell is usually not part of a multicellular organism (e.g., plant or animal). The host cell may be a non-human host cell.

Appropriate cloning and expression vectors for use with eukaryotic or mammalian cellular hosts and methods of cloning are known in the art.

The cells may be cultured under conditions that promote expression of the antibody. For example, a production bioreactor is used to culture the cells. The production bioreactor volume may be: (i) about 20,000 litres, about 10,000 litres; about 5,000 litres; about 2,000 litres; about 1,000 litres; or about 500 litres; or (ii) between 500 and 20,000 litres; between 500 and 10,000 litres; between 500 and 5,000 litres; between 1,000 and 10,000 litres, or between 2,000 and 10,000 litres. For example, the cells may be cultured in a production bioreactor at a pH of about 6.75 to pH 7.00. Alternatively, the cells may be cultured in a production bioreactor for about 12 to about 18 days. Alternatively, the cells may be cultured in a production bioreactor at a pH of about 6.75 to pH 7.00, for about 12 to about 18 days. This culture step may help to control the level of deamidated antibody variants, for example, to reduce the level of deamidated antibody variants.

The composition may be recovered and purified by conventional protein purification procedures. For example, the composition may be harvested directly from the culture medium. Harvest of the cell culture medium may be via clarification, for example by centrifugation and/or depth filtration. Recovery of the composition is followed by purification to ensure adequate purity.

A further embodiment of the disclosure is a recombinant host cell comprising the nucleic acid sequences or the expression vector described in any of the preceding embodiments. Another embodiment of the disclosure is a method for the production of a CD96 binding protein comprising culturing the host cell as described in the preceding embodiment under conditions suitable for expression of said nucleic acid sequence(s) or vector(s), whereby a polypeptide comprising the CD96 binding protein is produced. A further embodiment of the disclosure is the CD96 binding protein produced by the method for the production described in the preceding embodiment. Another embodiment of the disclosure in a cell line engineered to express the CD96 binding protein described in any one of the preceding embodiments.

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

“Sequence identity” as used herein is the degree of relatedness between two or more amino acid sequences, or two or more nucleic acid sequences, as determined by comparing the sequences. The comparison of sequences and determination of sequence identity may be accomplished using a mathematical algorithm; those skilled in the art will be aware of computer programs available to align two sequences and determine the percent identity between them. The skilled person will appreciate that different algorithms may yield slightly different results.

The term “specifically binds,” and grammatical variations thereof as used herein with respect to an antibody, is meant an antibody or antibody fragment which recognizes and binds with a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabelled A), in a reaction containing labelled “A” and the antibody, will reduce the amount of labelled A bound to the antibody.

The term “pharmaceutical composition” as used herein means a composition suitable for administration to a patient.

The pharmaceutical compositions described herein may comprise purified preparations of CD96 binding proteins as described herein.

For example, the pharmaceutical preparation may comprise a purified preparation of a CD96 binding as described herein in combination with a pharmaceutically acceptable carrier.

Typically, such pharmaceutical compositions comprise a pharmaceutically acceptable carrier as known and called for by acceptable pharmaceutical practice. Examples of such carriers include sterilized carriers, such as saline, Ringers solution, or dextrose solution, optionally buffered with suitable buffers to a pH within a range of 5 to 8.

Pharmaceutical compositions may be administered by injection or infusion (e.g., intravenous, intraperitoneal, intradermal, subcutaneous, intramuscular, or intraportal).

Such compositions are suitably free of visible particulate matter. Pharmaceutical compositions may comprise between 1 mg to 10 g of antigen binding protein, for example, between 5 mg and 1 g of antigen binding protein. Alternatively, the composition may comprise between 5 mg and 500 mg of antigen binding protein, for example, between 5 mg and 50 mg.

Methods for the preparation of such pharmaceutical compositions are well known to those skilled in the art. Pharmaceutical compositions may comprise between 1 mg to 10 g of antigen binding protein in unit dosage form, optionally together with instructions for use. Pharmaceutical compositions may be lyophilized (freeze dried) for reconstitution prior to administration according to methods well known or apparent to those skilled in the art.

Where antibodies have an IgG1 isotype, a chelator of copper, such as citrate (e.g., sodium citrate) or EDTA or histidine, may be added to the pharmaceutical composition to reduce the degree of copper-mediated degradation of antibodies of this isotype. Pharmaceutical compositions may also comprise a solubilizer, such as arginine, a surfactant/anti-aggregation agent such as polysorbate 80, and an inert gas such as nitrogen to replace vial headspace oxygen.

In one embodiment of the disclosure is a pharmaceutical composition comprising the CD96 binding protein as described in any of the preceding embodiments, and a pharmaceutically acceptable excipient. A further embodiment of the disclosure is a pharmaceutical composition comprising a therapeutically effective amount of a CD96 binding protein as described in any one of the preceding embodiments.

The term “anti-tumor effect” as used herein, refers to a biological effect which can be manifested by a reduction in the rate of tumor growth, decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the terms “cancer,” “neoplasm,” and “tumor” are used interchangeably and, in either the singular or plural form, refer to cells that have undergone a malignant transformation that makes them pathological to the host organism. Illustrative examples of cells that can be targeted by compositions and methods contemplated in particular embodiments include, but are not limited to the following cancers: synovial sarcoma, non-small-cell lung carcinoma (NSCLC), myxoid round cell liposarcoma (MRCLS), and multiple myeloma (MM). Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In an embodiment cancers that can be treated by the binding proteins of the invention can include solid tumours (e.g. recurrent, metastatic or advanced solid tumours). Examples of such solid tumours include ovarian, lung (e.g. NSCLC), gastric, bladder, colorectal, liver (e.g. HCC), renal (e.g. RCC), and head and neck squamous cell carcinoma (HNSCC).

The term “therapeutic” as used herein means a treatment and/or prophylaxis. A therapeutic effect is obtained by suppression, remission, or eradication of a disease state.

A “therapeutic effective amount” or “effective amount” as used herein, means an amount which provides a therapeutic or prophylactic benefit, or will elicit the biological or medical response of a tissue, system, or subject that is being sought by the researcher, veterinarian, medical doctor or other clinician. Therapeutically effective amounts and treatment regimes are generally determined empirically and may be dependent on factors, such as the age, weight, and health status of the patient and disease or disorder to be treated. Such factors are within the purview of the attending physician.

The term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

The terms “individual,” “subject,” and “patient” are used herein interchangeably. In one embodiment, the subject is a mammal, such as a primate, for example a marmoset or monkey, or a human. In a further embodiment, the subject is a human.

The dosage of antigen binding protein administered to a subject is generally between 1 μg/kg to 150 mg/kg, between 0.1 mg/kg and 100 mg/kg, between 0.5 mg/kg and 50 mg/kg, between 1 and 25 mg/kg, between about 0.3 mg/kg and about 3 mg/kg or between 1 and 10 mg/kg of the subject's body weight. For example, the dose may be 10 mg/kg, 30 mg/kg, or 60 mg/kg. The dose may also be from 10 mg/kg to 110 mg/mg 15 mg/kg to 25 mg/kg or 15 mg/kg to 100 mg/kg. The antigen binding protein may be administered, for example, parenterally, subcutaneously, intravenously, or intramuscularly. Doses may also be administered on a per subject basis such as about 20 mg per subject to about 750 mg per subject, about 75 mg per subject to about 750 mg per subject, about 20 mg per subject to about 200 mg per subject. The dose may be any discrete subrange with these dosage ranges. For example, the dose may also be administered subcutaneously on a per subject basis such as about 100 mg per subject (e.g., once every four weeks), or 300 mg per subject (or other doses administered may be subcutaneously with provided approximately the same, or comparable, bioavailability is achieved as with intravenous administration—e.g., three doses of 100 mg per subject to achieve a total dose administered subcutaneously of 300 mg per subject).

Ranges provided herein, of any type, include all values within a particular range described and values about an endpoint for a particular range.

If desired, the effective daily dose of an antibody or antigen binding protein of the disclosure (e.g., as a pharmaceutical composition) may be administered as two, three, four, five, six or more doses administered separately at appropriate intervals throughout the day, optionally, in unit dosage forms.

The administration of a dose may be by slow continuous infusion over a period of from 2 to 24 hours, such as from 2 to 12 hours, or from 2 to 6 hours. Such an administration may result in reduced side effects.

The administration of a dose may be repeated one or more times as necessary, for example, three times daily, once every day, once every 2 days, once a week, once a every 14 days, once a month, once every 3 months, once every 4 months, once every 6 months, or once every 12 months. The antigen binding proteins may be administered by maintenance therapy, for example once a week for a period of 6 months or more. The antigen binding proteins may be administered by intermittent therapy, for example, for a period of 3 to 6 months and then no dose for 3 to 6 months, followed by administration of antigen binding proteins again for 3 to 6 months, and so on, in a cycle.

For example, the dose may be administered subcutaneously, once every 14 or 28 days, in the form of multiple doses on each day of administration. In one embodiment, the dosage of the composition is 100 mg once every 4 weeks (28 days).

The antigen binding protein may be administered to the subject in such a way as to target therapy to a particular site.

The CD96 binding protein in the methods of the disclosure may be used in combination or co-administered with one or more other therapeutically active agents, such as antibodies, small molecule inhibitors, or in combination with a cell therapy. The term “co-administration” as used herein is meant either simultaneous administration or any manner of separate sequential administration of a CD96 binding protein, as described herein, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered by injection and another compound may be administered orally.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10th edition (Dec. 5, 2014), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule or anti-mitotic agents; platinum coordination complexes; alkylating agents; antibiotic agents; topoisomerase I inhibitors; topoisomerase II inhibitors; antimetabolites; hormones and hormonal analogues; signal transduction pathway inhibitors; non-receptor tyrosine kinase angiogenesis inhibitors; immunotherapeutic agents; proapoptotic agents; cell cycle signalling inhibitors; proteasome inhibitors; heat shock protein inhibitors; inhibitors of cancer metabolism; and cancer gene therapy agents.

Examples of a further active ingredient or ingredients for use in combination or co-administered with the presently disclosed CD96 binding proteins are anti-neoplastic agents. Examples of anti-neoplastic agents include, but are not limited to, chemotherapeutic agents; immuno-modulatory agents; immune-modulators; and immunostimulatory adjuvants.

The presently disclosed CD96 binding proteins may also be used in combination with anti-TIGIT antibodies. Such a combination may further enhance CD155/CD226 activation. A combination with anti-TIGIT antibody can be used to treat solid tumours such as kidney tumours e.g. renal cell carcinoma (RCC). An examples of such an anti-TIGIT antibody is tiragolumab.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Platinum coordination complexes are non-phase specific anti-cancer agents, which are interactive with DNA. The platinum complexes enter tumor cells, undergo aquation, and form intra- and interstrand crosslinks with DNA causing adverse biological effects to the tumor. Examples of platinum coordination complexes include, but are not limited to, cisplatin and carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Antibiotic anti-neoplastics are non-phase specific agents, which bind or intercalate with DNA. This action disrupts the ordinary function of the nucleic acids, leading to cell death. Examples of antibiotic anti-neoplastic agents include, but are not limited to, actinomycins such as dactinomycin; anthrocyclins such as daunorubicin and doxorubicin; and bleomycins.

Topoisomerase I inhibitors include, but are not limited to, camptothecins. The cytotoxic activity of camptothecins is believed to be related to its topoisomerase I inhibitory activity.

Topoisomerase II inhibitors include, but are not limited to, epipodophyllotoxins. Epipodophyllotoxins are phase specific anti-neoplastic agents derived from the mandrake plant. Epipodophyllotoxins typically affect cells in the S and G2 phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone; aminoglutethimide and other aromatase inhibitors such as anastrozole, letrazole, vorazole, and exemestane; progestrins such as megestrol acetate; estrogens, androgens, and anti-androgens such as flutamide, nilutamide, bicalutamide, cyproterone acetate and 5a-reductases such as finasteride and dutasteride; anti-estrogens such as tamoxifen, toremifene, raloxifene, droloxifene, iodoxyfene, as well as selective estrogen receptor modulators (SERMS); and gonadotropin-releasing hormone (GnRH) and analogues thereof, which stimulate the release of leutinizing hormone (LH) and/or follicle stimulating hormone (FSH), LHRH agonists, and antagonists such as goserelin acetate and leuprolide.

Signal transduction pathway inhibitors are those inhibitors, which block or inhibit a chemical process which evokes an intracellular change. As used herein, this change is cell proliferation or differentiation. Signal transduction inhibitors useful in the present invention include, but are not limited to, inhibitors of receptor tyrosine kinases, non-receptor tyrosine kinases, SH2/SH3 domain blockers, serine/threonine kinases, phosphatidyl inositol-3 kinases, myo-inositol signalling, and Ras oncogenes.

Several protein tyrosine kinases catalyze the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFR), tyrosine kinase with immunoglobulin-like and epidermal growth factor homology domains (TIE-2), insulin growth factor-I (IGFI) receptor, macrophage colony stimulating factor Cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath J. C., Exp. Opin. Ther. Patents, 10(6):803-818 (2000); Shawver L. K., et al., Drug Discov. Today, 2(2): 50-63 (1997); and Lofts, F. J. and Gullick W. J., “Growth factor receptors as targets.” in New Molecular Targets for Cancer Chemotherapy, Kerr D. J. and Workman P. (editors), (Jun. 27, 1994), CRC Press. Non-limiting examples of growth factor receptor inhibitors include pazopanib and sorafenib.

Tyrosine kinases, which are not growth factor receptor kinases, are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinha S. and Corey S. J., J. Hematother. Stem Cell Res., 8(5): 465-480 (2004) and Bolen, J. B., Brugge, J. S., Annu. Rev. Immunol., 15: 371-404 (1997).

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall T. E., J. Pharmacol. Toxicol. Methods, 34(3): 125-32 (1995).

Inhibitors of serine/threonine kinases include, but are not limited to, MAP kinase cascade blockers which include blockers of Raf kinases (rafk), Mitogen or Extracellular Regulated Kinase (MEKs), and Extracellular Regulated Kinases (ERKs); Protein kinase C family member blockers including blockers of PKCs (alpha, beta, gamma, epsilon, mu, lambda, iota, zeta); IkB kinases (IKKa, IKKb); PKB family kinases; AKT kinase family members; TGF beta receptor kinases; and mammalian target of rapamycin (mTOR) inhibitors, including, but not limited to rapamycin (FK506) and rapalogs, RAD001 or everolimus (AFINITOR), CCI-779 or temsirolimus, AP23573, AZD8055, WYE-354, WYE-600, WYE-687 and Pp121. Examples of inhibitors of serine/threonine kinases include, but are not limited to, trametinib, dabrafenib, and Akt inhibitors afuresertib and N-{(1S)-2-amino-1-[(3,4-difluorophenyl)methyl]ethyl}-5-chloro-4-(4-chloro-1-methyl-1H-pyrazol-5-yl)-2-furancarboxamide.

Inhibitors of phosphatidyl inositol 3-kinase family members including blockers of PI3-kinase, ATM, DNA-PK, and Ku are also useful in the present invention. Such kinases are discussed in Abraham R. T., Curr. Opin. Immunol., 8(3): 412-418 (1996); Canman C. E., and Lim D. S., Oncogene, 17(25): 3301-3308 (1998); Jackson S. P., Int. J. Biochem. Cell Biol., 29(7): 935-938 (1997); and Zhong H., et al., Cancer Res., 60(6): 1541-1545 (2000).

Also useful in the present invention are myo-inositol signalling inhibitors such as phospholipase C blockers and myo-inositol analogs. Such signal inhibitors are described in Powis G., and Kozikowski A., “Inhibitors of Myo-Inositol Signaling.” in New Molecular Targets for Cancer Chemotherapy, Kerr D. J. and Workman P. (editors), (Jun. 27, 1994), CRC Press.

Another group of signal transduction pathway inhibitors are inhibitors of Ras oncogene. Such inhibitors include inhibitors of farnesyltransferase, geranyl-geranyl transferase, and CAAX proteases as well as anti-sense oligonucleotides, ribozymes and other immunotherapies. Such inhibitors have been shown to block ras activation in cells containing wild type mutant ras, thereby acting as antiproliferation agents. Ras oncogene inhibition is discussed in Scharovsky O. G., et al., J. Biomed. Sci., 7(4): 292-298 (2000); Ashby M. N., Curr. Opin. Lipidol., 9(2): 99-102 (1998); and Bennett C.F. and Cowsert L. M., Biochem. Biophys. Acta., 1489(1): 19-30 (1999).

Antagonists to receptor kinase ligand binding may also serve as signal transduction inhibitors. This group of signal transduction pathway inhibitors includes the use of humanized antibodies or other antagonists to the extracellular ligand binding domain of receptor tyrosine kinases. Examples of antibody or other antagonists to receptor kinase ligand binding include, but are not limited to, cetuximab (ERBITUX), trastuzumab (HERCEPTIN); trastuzumab emtansine (KADCYLA); pertuzumab (PERJETA); ErbB inhibitors including lapatinib, erlotinib, and gefitinib; and 2C3 VEGFR2 specific antibody (see Brekken R.A., et al., Cancer Res., 60(18): 5117-5124 (2000)).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alpha beta3) that will inhibit angiogenesis; endostatin and angiostatin (non-RTK) may also prove useful in combination with the disclosed compounds. (See Bruns C. J., et al., Cancer Res., 60(11): 2926-2935 (2000); Schreiber A. B., et al., Science, 232(4755): 1250-1253 (1986); Yen L., et al., Oncogene, 19(31): 3460-3469 (2000)).

Agents used in immunotherapeutic regimens may also be useful in combination with the present invention. There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R.T., et al., Cancer Res., 60(13): 3569-3576 (2000); and Chen Y., et al., Cancer Res., 58(9): 1965-1971 (1998).

Agents used in proapoptotic regimens (e.g., Bcl-2 antisense oligonucleotides) may also be used in the combination of the present invention. Members of the Bcl-2 family of proteins block apoptosis. Upregulation of Bcl-2 has therefore been linked to chemoresistance. Studies have shown that the epidermal growth factor (EGF) stimulates anti-apoptotic members of the Bcl-2 family (i.e., Mcl-1). Therefore, strategies designed to downregulate the expression of Bcl-2 in tumors have demonstrated clinical benefit. Such proapoptotic strategies using the antisense oligonucleotide strategy for Bcl-2 are discussed in Waters J. S., et al., J. Clin. Oncol., 18(9): 1812-1823 (2000); and Kitada S., et al., Antisense Res. Dev., 4(2): 71-79 (1994).

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania G. R., and Chang Y. T., Exp. Opin. Ther. Patents, 10(2): 215-230 (2000). Further, p21WAF1/CIP1 has been described as a potent and universal inhibitor of cyclin-dependent kinases (Cdks) (Ball K. L., Prog. Cell Cycle Res., 3: 125-134 (1997)). Compounds that are known to induce expression of p21WAF1/CIP1 have been implicated in the suppression of cell proliferation and as having tumor suppressing activity (Richon V. M., et al., Proc. Natl. Acad. Sci. USA, 97(18): 10014-10019 (2000)), and are included as cell cycle signaling inhibitors. Histone deacetylase (HDAC) inhibitors are implicated in the transcriptional activation of p21WAF1/CIP1 (Vigushin D. M., and Coombes R. C., Anticancer Drugs, 13(1): 1-13 (2002)), and are suitable cell cycle signaling inhibitors for use in combination herein. Examples of such HDAC inhibitors include, but are not limited to vorinostat, romidepsin, panobinostat, valproic acid, and mocetinostat.

Proteasome inhibitors are drugs that block the action of proteasomes, cellular complexes that break down proteins, like the p53 protein. Several proteasome inhibitors are marketed or are being studied for the treatment of cancer. Suitable proteasome inhibitors for use in combination herein include, but are not limited to bortezomib, disulfiram, epigallocatechin gallate, salinosporamide A, and carfilzomib.

The 70 kilodalton heat shock proteins (Hsp70s) and 90 kilodalton heat shock proteins (Hsp90s) are a family of ubiquitously expressed heat shock proteins. Hsp70s and Hsp90s are over expressed certain cancer types. Several Hsp70 and Hsp90 inhibitors are being studied in the treatment of cancer. Examples of Hsp70 and Hsp90 inhibitors for use in combination herein include, but are not limited to tanespimycin and radicicol.

Many tumor cells show a markedly different metabolism from that of normal tissues. For example, the rate of glycolysis, the metabolic process that converts glucose to pyruvate, is increased, and the pyruvate generated is reduced to lactate, rather than being further oxidized in the mitochondria via the tricarboxylic acid (TCA) cycle. This effect is often seen even under aerobic conditions and is known as the Warburg Effect.

Lactate dehydrogenase A (LDH-A), an isoform of lactate dehydrogenase expressed in muscle cells, plays a pivotal role in tumor cell metabolism by performing the reduction of pyruvate to lactate, which can then be exported out of the cell. The enzyme has been shown to be upregulated in many tumor types. The alteration of glucose metabolism described in the Warburg effect is critical for growth and proliferation of cancer cells and knocking down LDH-A using RNA-i has been shown to lead to a reduction in cell proliferation and tumor growth in xenograft models (Tennant D. A., et al., Nat. Rev. Cancer, 10(4): 267-277 (2010); Fantin V. R., et al., Cancer Cell, 9(6): 425-434 (2006)).

High levels of fatty acid synthase (FAS) have been found in cancer precursor lesions. Pharmacological inhibition of FAS affects the expression of key oncogenes involved in both cancer development and maintenance. Alli P. M., et al., Oncogene, 24(1): 39-46 (2005).

Inhibitors of cancer metabolism, including inhibitors of LDH-A and inhibitors of fatty acid biosynthesis (or FAS inhibitors), are suitable for use in combination herein.

Cancer gene therapy involves the selective transfer of recombinant DNA/RNA using viral or nonviral gene delivery vectors to modify cancer calls for therapeutic purposes. Examples of cancer gene therapy include, but are not limited to suicide and oncolytic gene therapies, as well as adoptive T-cell therapies.

As used herein “immune-modulators” refer to any substance including monoclonal antibodies that affects the immune system. The CD96 binding proteins of the present invention can be considered immune-modulators. Immune-modulators can be used as anti-neoplastic agents for the treatment of cancer. For example, immune-modulators include, but are not limited to, antibodies or other antagonists to CTLA-4, such as ipilimumab (YERVOY), and PD-1, such as dostarlimab (JEMPERLI), nivolumab (OPDIVO), pembrolizumab (KEYTRUDA), and cemiplimab (LIBTAYO). Other immune-modulators include, but are not limited to, antibodies or other antagonists to PD-L1, OX-40, LAG3, TIM-3, 41BB, and GITR.

As used herein, “PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the aspects of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

In an embodiment, the chemical compound that is an inhibitor of PD-1 or PD-L1 is PCC0208025 (BMS202), BMS1166, BMS-8, BMS-37, BMS-202, BMS-230, BMS-242, BMS-1001, SB415286, vorinostat, panobinostat, azacitidine, decitabine, entitostat, JQ1, I-BET151, GSK503, or CA-170.

Examples of mAbs that bind to human PD-1, and useful in the various aspects and embodiments of the present invention, are described in U.S. Pat. Nos. 8,552,154; 8,354,509; 8,168,757; 8,008,449; 7,521,051; 7,488,802; WO2004072286; WO2004056875; and WO2004004771.

Other PD-1 antagonists useful in the any of the aspects and embodiments of the present invention include an immunoadhesin that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010027827 and WO2011066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

Nivolumab is a humanized monoclonal anti-PD-1 antibody commercially available as OPDIVO. Nivolumab is indicated for the treatment of some unresectable or metastatic melanomas. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of P13k/Akt pathway activation. Other names for nivolumab include: BMS-936558, MDX-1106, and ONO-4538. The amino acid sequence for nivolumab and methods of using and making are disclosed in U.S. Pat. No. 8,008,449.

Pembrolizumab is a humanized monoclonal anti-PD-1 antibody commercially available as KEYTRUDA. Pembrolizumab is indicated for the treatment of some unresectable or metastatic melanomas. The amino acid sequence of pembrolizumab and methods of using are disclosed in U.S. Pat. No. 8,168,757.

In one embodiment, the PD-1 antagonist comprises any one or a combination of the following CDRs:

CDRH1:
(SEQ ID NO: 151)
SYDMS
CDRH2:
(SEQ ID NO: 152)
TISGGGSYTYYQDSVKG
CDRH3:
(SEQ ID NO: 153)
PYYAMDY
CDRL1:
(SEQ ID NO: 154)
KASQDVGTAVA
CDRL2:
(SEQ ID NO: 155)
WASTLHT
CDRL3:
(SEQ ID NO: 156)
QHYSSYPWT

In one embodiment, the PD-1 antagonist comprises a heavy chain variable region CDR1 (“CDRH1”) comprising an amino acid sequence with one or two amino acid variation(s) (“CDR variant”) to the amino acid sequence set forth in SEQ ID NO:151.

In one embodiment, the PD-1 antagonist comprises a heavy chain variable region CDR2 (“CDRH2”) comprising an amino acid sequence with five or fewer, such as four or fewer, three or fewer, two or fewer, or one amino acid variation(s) (“CDR variant”) to the amino acid sequence set forth in SEQ ID NO:152. In a further embodiment, the CDRH2 comprises an amino acid sequence with one or two amino acid variation(s) to the amino acid sequence set forth in SEQ ID NO:152.

In one embodiment, the PD-1 antagonist comprises a heavy chain variable region CDR3 (“CDRH3”) comprising an amino acid sequence with one or two amino acid variation(s) (“CDR variant”) to the amino acid sequence set forth in SEQ ID NO:153.

In one embodiment, the PD-1 antagonist comprises a light chain variable region CDR1 (“CDRL1”) comprising an amino acid sequence with three or fewer, such as one or two amino acid variation(s) (“CDR variant”) to the amino acid sequence set forth in SEQ ID NO: 154.

In one embodiment, the PD-1 antagonist comprises a light chain variable region CDR2 (“CDRL2”) comprising an amino acid sequence with one or two amino acid variation(s) (“CDR variant”) to the amino acid sequence set forth in SEQ ID NO:155.

In one embodiment, the PD-1 antagonist comprises a light chain variable region CDR3 (“CDRL3”) comprising an amino acid sequence with three or fewer, such as one or two amino acid variation(s) (“CDR variant”) to the amino acid sequence set forth in SEQ ID NO:156. In a particular embodiment, the CDRL3 comprises an amino acid sequence with one amino acid variation to the amino acid sequence set forth in SEQ ID NO:156. In a further embodiment, the variant CDRL3 comprises the amino acid sequence set forth in SEQ ID NO:157.

In one embodiment, the PD-1 antagonist comprises a CDRH1 comprising an amino acid sequence with up to one amino acid variation to the amino acid sequence set forth in SEQ ID NO:151; a CDRH2 comprising an amino acid sequence with up to five amino acid variations to the amino acid sequence set forth in SEQ ID NO:152; a CDRH3 comprising an amino acid sequence with up to one amino acid variation to the amino acid sequence set forth in SEQ ID NO:153; a CDRL1 comprising an amino acid sequence with up to three amino acid variations to the amino acid sequence set forth in SEQ ID NO:154; a CDRL2 comprising an amino acid sequence with up to one amino acid variation to the amino acid sequence set forth in SEQ ID NO:155; and/or a CDRL3 comprising an amino acid sequence with up to three amino acid variations to the amino acid sequence set forth in SEQ ID NO:156.

In one embodiment of the present invention the PD-1 antagonist comprises CDRH1 (SEQ ID NO:151), CDRH2 (SEQ ID NO:152), and CDRH3 (SEQ ID NO:153) in the heavy chain variable region having the amino acid sequence set forth in SEQ ID NO: 158. In some embodiments, the anti-PD-1 antibodies of the present invention comprise a heavy chain variable region having at least 90% sequence identity to SEQ ID NO: 158. Suitably, the PD-1 antagonists of the present invention may comprise a heavy chain variable region having about 85%, 86%, 87%, 88%, 89%, 90%, 91%, 92%, 93%, 94%, 95%, 96%, 97%, 98%, 99%, or 100% sequence identity to SEQ ID NO:158.

PD-1 antagonist heavy chain (VH)
variable region:
(SEQ ID NO: 158)
EVQLLESGGGLVQPGGSLRLSCAASGFTFSSYDMSWVRQAPGKGL
EWVSTISGGGSYTYYQDSVKGRFTISRDNSKNTLYLQMNSLRAE
DTAVYYC ASPYYAMDYWGQGTTVTVSS

In one embodiment, the PD-1 antagonist comprises a heavy chain variable region (“VH”) comprising an amino acid sequence with at least about 90%, 91%, 92%, 93%, 94%, 95%, 96%, 97%, 98%, 99% or 100% sequence identity to the amino acid sequence set forth in SEQ ID NO:158 In one embodiment, the VH comprises an amino acid sequence with at least one amino acid variation to the amino acid sequence set forth in SEQ ID NO:158, such as between 1 and 5, such as between 1 and 3, in particular up to 2 amino acid variations to the amino acid sequence set forth in SEQ ID NO:158.

In one embodiment of the present invention the PD-1 antagonist comprises CDRL1 (SEQ ID NO:154), CDRL2 (SEQ ID NO:155), and CDRL3 (SEQ ID NO:156) in the light chain variable region having the amino acid sequence set forth in SEQ ID NO:159. In one embodiment, a PD-1 antagonist of the present invention comprises the heavy chain variable region of SEQ ID NO:158 and the light chain variable region of SEQ ID NO:159.

In some embodiments, the PD-1 antagonists of the present invention comprise a light chain variable region having at least 90% sequence identity to the amino acid sequence set forth in SEQ ID NO:159. Suitably, the PD-1 antagonists of the present invention may comprise a light chain variable region having about 85%, 86%, 87%, 88%, 89%, 90%, 91%, 92%, 93%, 94%, 95%, 96%, 97%, 98%, 99%, or 100% sequence identity to SEQ ID NO:159.

PD-1 antagonist light chain (VL)
variable region:
(SEQ ID NO: 159)
DIQLTQSPSFLSAYVGDRVTITCKASQDVGTAVAWYQQKPGKAPK
LLIYWASTLHTGVPSRFSGSGSGTEFTLTISSLQPEDFATYYCQ
HYSSYPWTF GQGTKLEIK

In one embodiment, the PD-1 antagonist comprises a light chain variable region (“VL”) comprising an amino acid sequence with at least about 90%, 91%, 92%, 93%, 94%, 95%, 96%, 97%, 98%, 99% or 100% sequence identity to the amino acid sequence set forth in SEQ ID NO:159. In one embodiment, the VL comprises an amino acid sequence with at least one amino acid variation to the amino acid sequence set forth in SEQ ID NO:159, such as between 1 and 5, such as between 1 and 3, in particular up to 2 amino acid variations to the amino acid sequence set forth in SEQ ID NO:159.

In one embodiment, the PD-1 antagonist comprises a VH comprising an amino acid sequence with at least about 90%, 91%, 92%, 93%, 94%, 95%, 96%, 97%, 98%, 99% or 100% sequence identity to the amino acid sequence set forth in SEQ ID NO:158; and a VL comprising an amino acid sequence with at least about 90%, 91%, 92%, 93%, 94%, 95%, 96%, 97%, 98%, 99% or 100% sequence identity to the amino acid sequence set forth in SEQ ID NO: 159. In one embodiment, the PD-1 antagonist comprises a VH at least about 90% identical to the amino acid sequence of SEQ ID NO:158 and/or a VL at least about 90% identical to the amino acid sequence of SEQ ID NO:159.

In one embodiment, a PD-1 antagonist comprises a VH with the amino acid sequence set forth in SEQ ID NO:158, and a VL with the amino acid sequence set forth in SEQ ID NO:159.

In one embodiment, the PD-1 antagonist is a monoclonal antibody comprising a heavy chain (HC) amino acid sequence having at least 90%, 91%, 92,%, 93%, 94%, 95%, 96%, 97%, 98%, 99% or 100% sequence identity to the amino acid sequence set forth in SEQ ID NO:160.

(SEQ ID NO: 160)
EVQLLESGGGLVQPGGSLRLSCAASGFTFSSYDMSWVRQAPGKGL
EWVSTISGGGSYTYYQDSVKGRFTISRDNSKNTLYLQMNSLRAE
DTAVYYCASPYYAMDYWGQGTTVTVSSASTKGPSVFPLAPCSRST
SESTAALGCLVKDYFPEPVTVSWNSGALTSGVHTFPAVLQSSGL
YSLSSVVTVPSSSLGTKTYTCNVDHKPSNTKVDKRVESKYGPPC
PPCPAPEFLGGPSVFLFPPKPKDTLMISRTPEVTCVVVDVSQEDP
EVQFNWYVDGVEVHNAKTKPREEQFNSTYRVVSVLTVLHQDWLN
GKEYKCKVSNKGLPSSIEKTISKAKGQPREPQVYTLPPSQEEMT
KNQVSLTCLVKGFYPSDIAVEWESNGQPENNYKTTPPVLDSDGSF
FLYSRLTVDKSRWQEGNVFSCSVMHEALHNHYTQKSLSLSLGK

In one embodiment, the HC comprises an amino acid sequence with at least one amino acid variation to the amino acid sequence set forth in SEQ ID NO: 160, such as between 1 and 10, such as between 1 and 7, in particular up to 6 amino acid variations to the amino acid sequence set forth in SEQ ID NO:160. In a further embodiment, the HC comprises one, two, three, four, five, six or seven amino acid variations to the amino acid sequence set forth in SEQ ID NO:160.

In one embodiment, the HC chain comprises a variation at position 380 and/or 385 of SEQ ID NO:160. The asparagine residues at these positions may be modified, e.g. by deamidation (conversion of a asparagine (N) residue into an aspartate (D) residue). Therefore, in one embodiment, the HC comprises an amino acid sequence of SEQ ID NO: 162 (N380D), SEQ ID NO:163 (N385D) or SEQ ID NO:164 (N380D and N385D).

In one embodiment, the PD-1 antagonist is a monoclonal antibody comprising a light chain (LC) amino acid sequence having at least 90%, 91%, 92,%, 93%, 94%, 95%, 96%, 97%, 98%, 99% or 100% sequence identity to the amino acid sequence set forth in SEQ ID NO:161.

(SEQ ID NO: 161)
DIQLTQSPSFLSAYVGDRVTITCKASQDVGTAVAWYQQKPGKAPK
LLIYWASTLHTGVPSRFSGSGSGTEFTLTISSLQPEDFATYYCQ
HYSSYPWTFGQGTKLEIKRTVAAPSVFIFPPSDEQLKSGTASVVC
LLNNFYPREAKVQWKVDNALQSGNSQESVTEQDSKDSTYSLSST
LTLSKADYEKHKVYACEVTHQGLSSPVTKSFNRGEC

In one embodiment, the LC comprises an amino acid sequence with at least one amino acid variation to the amino acid sequence set forth in SEQ ID NO: 161, such as between 1 and 10, such as between 1 and 5, in particular up to 3 amino acid variations to the amino acid sequence set forth in SEQ ID NO:161. In a further embodiment, the LC comprises one, two or three amino acid variations to the amino acid sequence set forth in SEQ ID NO:161.

In one embodiment, the PD-1 antagonist comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO:160; and a LC comprising an amino acid sequence with at least about 90%, 91%, 92%, 93%, 94%, 95%, 96%, 97%, 98%, 99% or 100% sequence identity to the amino acid sequence set forth in SEQ ID NO:161. Therefore, the antibody is an antibody with a heavy chain at least about 90% identical to the heavy chain amino acid sequence of SEQ ID NO:160 and/or with a light chain at least about 90% identical to the light chain amino acid sequence of SEQ ID NO:161.

In one embodiment, the PD-1 antagonist comprises a heavy chain amino acid sequence at least about 90% identical to the amino acid sequence of SEQ ID NO:160 and/or a light chain amino acid sequence at least about 90% identical to the amino acid sequence of SEQ ID NO:161.

In one embodiment, the PD-1 antagonist comprises a heavy chain sequence of SEQ ID NO:160 and a light chain sequence of SEQ ID NO:161. In one embodiment, the antibody is dostarlimab comprising a heavy chain sequence of SEQ ID NO:160 and a light chain sequence of SEQ ID NO: 161.

Anti-PD-L1 antibodies and methods of making the same are known in the art. Such antibodies to PD-L1 may be polyclonal or monoclonal, and/or recombinant, and/or humanized. PD-L1 antibodies are in development as immuno-modulatory agents for the treatment of cancer.

Exemplary PD-L1 antibodies are disclosed in U.S. Pat. Nos. 9,212,224; 8,779,108; 8,552,154; 8,383,796; 8,217,149; US Patent Publication No. 20110280877; WO2013079174; and WO2013019906. Additional exemplary antibodies to PD-L1 (also referred to as CD274 or B7-H1) and methods for use are disclosed in U.S. Pat. Nos. 8,168,179; 7,943,743; 7,595,048; WO2014055897; WO2013019906; and WO2010077634. Specific anti-human PD-L1 monoclonal antibodies useful as a PD-1 antagonist in the treatment method, medicaments and uses of the present invention include MPDL3280A, BMS-936559, MEDI4736, MSB0010718C.

Atezolizumab is a fully humanized monoclonal anti-PD-L1 antibody commercially available as TECENTRIQ. Atezolizumab is indicated for the treatment of some locally advanced or metastatic urothelial carcinomas. Atezolizumab blocks the interaction of PD-L1 with PD-1 and CD80. Other exemplary PD-L1 antibodies include avelumab (BAVENCIO), durvalumab (IMFINZI)

CD134, also known as OX40, is a member of the TNFR-superfamily of receptors which is not constitutively expressed on resting naïve T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX-40 antibodies, OX-40 fusion proteins and methods of using them are disclosed in U.S. Pat. Nos. 7,504,101; 7,758,852; 7,858,765; 7,550,140; 7,960,515; WO2012027328; WO2013028231.

Additional examples of a further active ingredient or ingredients (anti-neoplastic agent) for use in combination or co-administered with the presently disclosed CD96 binding proteins are antibodies or other antagonists to CD20, retinoids, or other kinase inhibitors. Examples of such antibodies or antagonists include, but are not limited to rituximab (RITUXAN and MABTHERA), ofatumumab (ARZERRA), and bexarotene (TARGRETIN).

Additional examples of a further active ingredient or ingredients (anti-neoplastic agent) for use in combination or co-administered with the presently disclosed CD96 binding proteins are Toll-like Receptor 4 (TLR4) antagonists, including but not limited to aminoalkyl glucosaminide phosphates (AGPs).

AGPs are known to be useful as vaccine adjuvants and immunostimulatory agents for stimulating cytokine production, activating macrophages, promoting innate immune response, and augmenting antibody production in immunized animals. AGPs are synthetic ligands of TLR4. AGPs and their immunomodulating effects via TLR4 are disclosed in patent publications such as WO 2006016997, WO 2001090129, and/or U.S. Pat. No. 6,113,918 and have been reported in the literature. Additional AGP derivatives are disclosed in U.S. Pat. Nos. 7,129,219, 6,911,434, and 6,525,028. Certain AGPs act as agonists of TLR4, while others are recognized as TLR4 antagonists.

Additional non-limiting examples of a further active ingredient or ingredients (anti-neoplastic agent) for use in combination or co-administered with the presently disclosed CD96 binding proteins are antibodies to ICOS.

CDRs for murine antibodies to human ICOS having agonist activity are shown in PCT/EP2012/055735 (WO 2012131004). Antibodies to ICOS are also disclosed in WO 2008137915, WO 2010056804, EP 1374902, EP1374901, and EP1125585.

Additional examples of a further active ingredient or ingredients (anti-neoplastic agent) for use in combination or co-administered with the presently disclosed CD96 binding proteins are poly ADP ribose polymerase (PARP) inhibitors. Non-limiting examples of such inhibitors include niraparib, olaparib, rucaparib, and talazoparib.

Additional non-limiting examples of a further active ingredient or ingredients (anti-neoplastic agent) for use in combination or co-administered with the presently disclosed CD96 binding proteins are STING modulating compounds, CD39 inhibitors and A2a and A2a adenosine antagonists.

Select anti-neoplastic agents that may be used in combination with CD96 binding proteins or a pharmaceutically acceptable salt thereof, include but are not limited to: abarelix, abemaciclib, abiraterone, afatinib, aflibercept, aldoxorubicin, alectinib, alemtuzumab, arsenic trioxide, asparaginase, axitinib, AZD-9291, belinostat, bendamustine, bevacizumab, blinatumomab, bosutinib, brentuximab vedotin, cabazitaxel, cabozantinib, capecitabine, ceritinib, clofarabine, cobimetinib, crizotinib, daratumumab, dasatinib, degarelix, denosumab, dinutuximab, docetaxel, elotuzumab, entinostat, enzalutamide, epirubicin, eribulin, filgrastim, flumatinib, fulvestrant, fruquintinib, gemtuzumab ozogamicin, ibritumomab, ibrutinib, idelalisib, imatinib, irinotecan, ixabepilone, ixazomib, lenalidomide, lenvatinib, leucovorin, mechlorethamine, necitumumab, nelarabine, netupitant, nilotinib, obinutuzumab, olaparib, omacetaxine, osimertinib, oxaliplatin, paclitaxel, palbociclib, palonosetron, panitumumab, pegfilgrastim, peginterferon alfa-2b, pemetrexed, plerixafor, pomalidomide, ponatinib, pralatrexate, quizartinib, radium-223, ramucirumab, regorafenib, rolapitant, rucaparib, sipuleucel-T, sonidegib, sunitinib, talimogene laherparepvec, tipiracil, topotecan, trabectedin, trifluridine, triptorelin, uridine, vandetanib, velaparib, vemurafenib, venetoclax, vincristine, vismodegib, and zoledronic acid.

As used herein, “PVRIG antagonist” means any chemical compound or biological molecule that blocks binding of PVRL2, such as PVRL2 expressed on a cancer cell, to PVRIG, such as PVRIG expressed on an immune cell (T cell, B cell or NKT cell). Alternative names or synonyms for PVRIG and its ligands include: C7orf15, C7orf15MGC138295, CD112R, MGC104322, MGC138297, MGC2463, poliovirus receptor related immunoglobulin domain containing, Poliovirus receptor-related immunoglobulin domain-containing protein, and transmembrane protein PVRIG for PVRIG; and NECTIN2, CD112, HVEB, PRR2, PVRR2, and nectin cell adhesion molecule 2 for PVRL2.

PVRIG antagonists useful in the any of the aspects of the present invention include a monoclonal antibody (mAb), or antigen binding fragment thereof, which specifically binds to PVRIG or PVRL2, and preferably specifically binds to human PVRIG or PVRL2. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PVRIG, and are useful in the various aspects and embodiments of the present disclosure, are described, for example, in WO 2020/018879 and WO 2016/134333.

Thus, additional examples of a further active ingredient or ingredients (anti-neoplastic agent) for use in combination or co-administered with the presently disclosed CD96 binding proteins are antibodies or other antagonists to PVRIG. Examples of such antibodies or antagonists include, but are not limited to, anti-PVRIG antibodies such as SRF813 (Surface Oncology) or COM701 (CGEN-15029) (Compugen).

In an embodiment, the anti-PVRIG antibody comprises: (a) CDRH1 comprising SEQ ID NO: 181, CDRH2 comprising SEQ ID NO: 182, CDRH3 comprising SEQ ID NO: 183, CDRL1 comprising SEQ ID NO: 184, CDRL2 comprising SEQ ID NO: 185, and CDRL3 comprising SEQ ID NO: 186; (b) CDRH1 comprising SEQ ID NO: 187, CDRH2 comprising SEQ ID NO: 188, CDRH3 comprising SEQ ID NO: 189, CDRL1 comprising SEQ ID NO: 190, CDRL2 comprising SEQ ID NO: 191, and CDRL3 comprising SEQ ID NO: 192; (c) CDRH1 comprising SEQ ID NO: 193, CDRH2 comprising SEQ ID NO: 194, CDRH3 comprising SEQ ID NO: 195, CDRL1 comprising SEQ ID NO: 196, CDRL2 comprising SEQ ID NO: 197, and CDRL3 comprising SEQ ID NO: 198; (d) CDRH1 comprising SEQ ID NO: 199, CDRH2 comprising SEQ ID NO: 200, CDRH3 comprising SEQ ID NO: 201, CDRL1 comprising SEQ ID NO: 202, CDRL2 comprising SEQ ID NO: 203, and CDRL3 comprising SEQ ID NO: 204; (e) CDRH1 comprising SEQ ID NO: 205, CDRH2 comprising SEQ ID NO: 206, CDRH3 comprising SEQ ID NO: 207, CDRL1 comprising SEQ ID NO: 208, CDRL2 comprising SEQ ID NO: 209, and CDRL3 comprising SEQ ID NO: 210; (f) CDRH1 comprising SEQ ID NO: 211, CDRH2 comprising SEQ ID NO: 212, CDRH3 comprising SEQ ID NO: 213, CDRL1 comprising SEQ ID NO: 214, CDRL2 comprising SEQ ID NO: 215, and CDRL3 comprising SEQ ID NO: 216; (g) CDRH1 comprising SEQ ID NO: 217, CDRH2 comprising SEQ ID NO: 218, CDRH3 comprising SEQ ID NO: 219, CDRL1 comprising SEQ ID NO: 220, CDRL2 comprising SEQ ID NO: 221, and CDRL3 comprising SEQ ID NO: 222; (h) CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228; (i) CDRH1 comprising SEQ ID NO: 229, CDRH2 comprising SEQ ID NO: 230, CDRH3 comprising SEQ ID NO: 231, CDRL1 comprising SEQ ID NO: 232, CDRL2 comprising SEQ ID NO: 233, and CDRL3 comprising SEQ ID NO: 234; (j) CDRH1 comprising SEQ ID NO: 235, CDRH2 comprising SEQ ID NO: 236, CDRH3 comprising SEQ ID NO: 237, CDRL1 comprising SEQ ID NO: 238, CDRL2 comprising SEQ ID NO: 239, and CDRL3 comprising SEQ ID NO: 240; (k) CDRH1 comprising SEQ ID NO: 241, CDRH2 comprising SEQ ID NO: 242, CDRH3 comprising SEQ ID NO: 243, CDRL1 comprising SEQ ID NO: 244, CDRL2 comprising SEQ ID NO: 245, and CDRL3 comprising SEQ ID NO: 246; (l) CDRH1 comprising SEQ ID NO: 247, CDRH2 comprising SEQ ID NO: 248, CDRH3 comprising SEQ ID NO: 249, CDRL1 comprising SEQ ID NO: 250, CDRL2 comprising SEQ ID NO: 251, and CDRL3 comprising SEQ ID NO: 252; (m) CDRH1 comprising SEQ ID NO: 253, CDRH2 comprising SEQ ID NO: 254, CDRH3 comprising SEQ ID NO: 255, CDRL 1 comprising SEQ ID NO: 256, CDRL2 comprising SEQ ID NO: 257, and CDRL3 comprising SEQ ID NO: 258; or (n) CDRH1 comprising SEQ ID NO: 259, CDRH2 comprising SEQ ID NO: 260, CDRH3 comprising SEQ ID NO: 261,
CDRL1 comprising SEQ ID NO: 262, CDRL2 comprising SEQ ID NO: 263, and CDRL3 comprising SEQ ID NO: 264.

In an embodiment, the anti-PVRIG antibody comprises: (a) VH comprising SEQ ID NO: 265 and VL comprising SEQ ID NO: 266; (b) VH comprising SEQ ID NO: 267 and VL comprising SEQ ID NO: 268; (c) VH comprising SEQ ID NO: 269 and VL comprising SEQ ID NO: 270; (d) VH comprising SEQ ID NO: 271 and VL comprising SEQ ID NO: 272; (e) VH comprising SEQ ID NO: 273 and VL comprising SEQ ID NO: 274; (f) VH comprising SEQ ID NO: 275 and VL comprising SEQ ID NO: 276; (g) VH comprising SEQ ID NO: 277 and VL comprising SEQ ID NO: 278; (h) VH comprising SEQ ID NO: 279 and VL comprising SEQ ID NO: 280; (i) VH comprising SEQ ID NO: 281 and VL comprising SEQ ID NO: 282; (j) VH comprising SEQ ID NO: 283 and VL comprising SEQ ID NO: 284; (k) VH comprising SEQ ID NO: 285 and VL comprising SEQ ID NO: 286; (l) VH comprising SEQ ID NO: 287 and VL comprising SEQ ID NO: 288; (m) VH comprising SEQ ID NO: 289 and VL comprising SEQ ID NO: 290; or (n) VH comprising SEQ ID NO: 291 and VL comprising SEQ ID NO: 292.

In an embodiment, the anti-PVRIG antibody comprises: CDRH1 comprising SEQ ID NO: 293, CDRH2 comprising SEQ ID NO: 294, CDRH3 comprising SEQ ID NO: 294, CDRL1 comprising SEQ ID NO: 296, CDRL2 comprising SEQ ID NO: 297, and CDRL3 comprising SEQ ID NO: 298.

In an embodiment, the anti-PVRIG antibody comprises: (a) VH comprising SEQ ID NO: 299 and VL comprising SEQ ID NO: 300; or (b) VH comprising SEQ ID NO: 301 and VL comprising SEQ ID NO: 302.

As used herein, “TIGIT antagonist” means any chemical compound or biological molecule that inhbits the activity of TIGIT by interfering with its ligand binding and/or signalling activity. In embodiments, a TIGIT antagonist of the disclosure interferes with TIGIT binding to CD155/NECL5 and/or signalling induced by CD155/NECL5.

TIGIT antagonists useful in the any of the aspects of the present invention include a monoclonal antibody (mAb), or antigen binding fragment thereof, which specifically binds to TIGIT, e.g., TIGIT expressed on the surface of a T cell, a NK cell, or a NKT cell. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

In an embodiment, the anti-TIGIT antibody comprises a heavy chain sequence comprising SEQ ID NO: 392 and a light chain sequence comprising SEQ ID NO: 393. In a further embodiment, the anti-TIGIT antibody comprises: (a) CDRH1 comprising SEQ ID NO: 320, CDRH2 comprising SEQ ID NO: 321, CDRH3 comprising SEQ ID NO: 322, CDRL1 comprising SEQ ID NO: 317, CDRL2 comprising SEQ ID NO: 318, and CDRL3 comprising SEQ ID NO: 319. In still another embodiment, the anti-TIGIT antibody comprises: (a) CDRH1 comprising SEQ ID NO: 326, CDRH2 comprising SEQ ID NO: 327, CDRH3 comprising SEQ ID NO: 328, CDRL1 comprising SEQ ID NO: 323, CDRL2 comprising SEQ ID NO: 324, and CDRL3 comprising SEQ ID NO: 325. In yet another embodiment, the anti-TIGIT antibody comprises a heavy chain variable sequence comprising a sequence selected from SEQ ID NO: 315 and SEQ ID NO: 316, and a light chain variable sequence comprising a sequence selected from SEQ ID NO: 313 and SEQ ID NO: 314.

In an embodiment, the anti-TIGIT antibody comprises a heavy chain variable sequence comprising SEQ ID NO: 329 and a light chain variable sequence comprising SEQ ID NO: 330. In another embodiment, the anti-TIGIT antibody comprises: (a) CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In still another embodiment, the anti-TIGIT antibody comprises a heavy chain variable sequence comprising a sequence selected from SEQ ID NO: 337, SEQ ID NO: 339 and SEQ ID NO: 341, and a light chain variable sequence comprising a sequence selected from SEQ ID NO: 338, SEQ ID NO: 340, and SEQ ID NO: 342.

In an embodiment, the anti-TIGIT antibody comprises a CDRH1 sequence comprising a sequence selected from SEQ ID NO: 320, SEQ ID NO: 326, and SEQ ID NO: 334, a CDRH2 sequence comprising a sequence selected from SEQ ID NO: 321, SEQ ID NO: 327, and SEQ ID NO: 335, and a CDRH3 sequence comprising a sequence selected from SEQ ID NO: 322, SEQ ID NO: 328, and SEQ ID NO: 336.

In an embodiment, the anti-TIGIT antibody comprises a CDRL1 sequence comprising a sequence selected from SEQ ID NO: 317, SEQ ID NO: 323, and SEQ ID NO: 331, a CDRL2 sequence comprising a sequence selected from SEQ ID NO: 318, SEQ ID NO: 324, and SEQ ID NO: 332, and a CDRL3 sequence comprising a sequence selected from SEQ ID NO: 319, SEQ ID NO: 325, and SEQ ID NO: 333.

In an embodiment, the anti-TIGIT antibody comprises a heavy chain variable sequence comprising a sequence selected from SEQ ID NO: 315, SEQ ID NO: 316, SEQ ID NO: 329, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 395, SEQ ID NO: 402, SEQ ID NO: 404, SEQ ID NO: 406, SEQ ID NO: 408, SEQ ID NO: 410, SEQ ID NO: 411, SEQ ID NO: 412, SEQ ID NO: 413, SEQ ID NO: 414, SEQ ID NO: 415, SEQ ID NO: 416, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 437, SEQ ID NO: 439, SEQ ID NO: 441, SEQ ID NO: 443, SEQ ID NO: 445, SEQ ID NO: 446, SEQ ID NO: 447, and SEQ ID NO: 448.

In still further embodiments, the anti-TIGIT antibody comprises a light chain variable sequence comprising a sequence selected from SEQ ID NO: 330, SEQ ID NO: 313, SEQ ID NO: 314, SEQ ID NO: 338, SEQ ID NO: 340, SEQ ID NO: 342, SEQ ID NO: 345, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 394, SEQ ID NO: 403, SEQ ID NO: 405, SEQ ID NO: 407, SEQ ID NO: 409, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 438, SEQ ID NO: 440, SEQ ID NO: 442, and SEQ ID NO: 444.

In a still further embodiment, the anti-TIGIT antibody comprises a heavy chain and a light chain variable sequence as shown in Table X:

TABLE X

| VH | VL |
|---|---|
| SEQ ID NO: | SEQ ID NO: |
| 315 | 313 |
| 316 | 314 |
| 329 | 330 |
| 337 | 338 |
| 339 | 340 |
| 341 | 342 |
| 343 | 345 |
| 344 | 345 |
| 352 | 353 |
| 354 | 355 |
| 356 | 357 |
| 358 | 359 |
| 360 | 361 |
| 362 | 363 |
| 364 | 365 |
| 366 | 367 |
| 368 | 369 |
| 370 | 371 |
| 372 | 373 |
| 374 | 375 |
| 376 | 377 |
| 378 | 379 |
| 380 | 381 |
| 382 | 383 |
| 384 | 385 |
| 386 | 387 |
| 388 | 389 |
| 390 | 391 |
| 395 | 394 |
| 402 | 403 |
| 404 | 405 |
| 406 | 407 |
| 408 | 409 |
| 437 | 438 |
| 439 | 440 |
| 441 | 442 |
| 443 | 444 |

In an embodiment, the chemical compound that is a TIGIT antagonist is liothyronine, OMP-313 M32, or MTIG7192A.

Combinations of two or more of a PD-1 antagonist, a TIGIT antagonist, a PVRIG antagonist, and/or CD96 antagonist as disclosed herein are provided by the present disclosure.

In one embodiment, a combination may comprise a PD-1 antagonist and a TIGIT antagonist. In another embodiment, a combination may comprise a PD-1 antagonist and a PVRIG antagonist. In another embodiment, a combination may comprise a PD-1 antagonist and a CD96 antagonist. In another embodiment, a combination may comprise a TIGIT antagonist and a PVRIG antagonist. In another embodiment, a combination may comprise a TIGIT antagonist and a CD96 antagonist. In another embodiment, a combination may comprise a PVRIG antagonist and a CD96 antagonist.

A combination of two or more antagonists disclosed herein may be expressed as a ratio of the two or more antagonists. For example, a combination of antagonists disclosed herein may be provided as a ratio of a fixed dose of an antagonist to a fixed dose of another antagonist (e.g., a 1:1 ratio of a fixed dose of a PD-1 antagonist to a fixed dose of a TIGIT antagonist).

In an embodiment, the ratio of PD-1 antagonist to TIGIT antagonist may be at least about 1:0.25 to at least about 1:2. In a further embodiment, the ratio of PD-1 antagonist to TIGIT antagonist may be at least about 1:0.25, at least about 1:0.30, at least about 1:0.35, at least about 1:0.40, at least about 1:0.45, at least about 1:0.50, at least about 1:0.55, at least about 1:0.60, at least about 1:0.65, at least about 1:0.70, at least about 1:0.75, at least about 1:0.80, at least about 1:0.85, at least about 1:0.90, at least about 1:0.95, at least about 1:1, at least about 1:1.05, at least about 1:1.10, at least about 1:1.15, at least about 1:1.20, at least about 1:1.25, at least about 1:1.30, at least about 1:1.35, at least about 1:1.40, at least about 1:1.45, at least about 1:1.50, at least about 1:1.55, at least about 1:1.60, at least about 1:1.65, at least about 1:1.70, at least about 1:1.75, at least about 1:1.80, at least about 1:1.85, at least about 1:1.90, at least about 1:1.95, or at least about 1.2. In yet a further embodiment, the ratio of PD-1 antagonist to TIGIT antagonist may be 1:0.25, 1:0.26, 1:0.27, 1:0.28, 1:0.29, 1:0.30, 1:0.31, 1:0.32, 1:0.33, 1:0.34, 1:0.35, 1:0.36, 1:0.37, 1:0.38, 1:0.39, 1:0.40, 1:0.41, 1:0.42, 1:0.43, 1:0.44, 1:0.45, 1:0.46, 1:0.47, 1:0.48, 1:0.49, 1:0.50, 1:0.51, 1:0.52, 1:0.53, 1:0.54, 1:0.55, 1:0.56, 1:0.57, 1:0.58, 1:0.59, 1:0.60, 1:0.61, 1:0.62, 1:0.63, 1:0.64, 1:0.65, 1:0.66, 1:0.67, 1:0.68, 1:0.69, 1:0.70, 1:0.71, 1:0.72, 1:0.73, 1:0.74, 1:0.75, 1:0.76, 1:0.77, 1:0.78, 1:0.79, 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, 1:1.20, 1:1.21, 1:1.22, 1:1.23, 1:1.24, 1:1.25, 1:1.26, 1:1.27, 1:1.28, 1:1.29, 1:1.30, 1:1.31, 1:1.32, 1:1.33, 1:1.34, 1:1.35, 1:1.36, 1:1.37, 1:1.38, 1:1.39, 1:1.40, 1:1.41, 1:1.42, 1:1.43, 1:1.44, 1:1.45, 1:1.46, 1:1.47, 1:1.48, 1:1.49, 1:1.50, 1:1.51, 1:1.52, 1:1.53, 1:1.54, 1:1.55, 1:1.56, 1:1.57, 1:1.58, 1:1.59, 1:1.60, 1:1.61, 1:1.62, 1:1.63, 1:1.64, 1:1.65, 1:1.66, 1:1.67, 1:1.68, 1:1.69, 1:1.70, 1:1.71, 1:1.72, 1:1.73, 1:1.74, 1:1.75, 1:1.76, 1:1.77, 1:1.78, 1:1.79, 1:1.80, 1:1.81, 1:1.82, 1:1.83, 1:1.84, 1:1.85, 1:1.86, 1:1.87, 1:1.88, 1:1.89, 1:1.90, 1:1.91, 1:1.92, 1:1.93, 1:1.94, 1:1.95, 1:1.96, 1:1.97, 1:1.98, 1:1.99, or 1:2.

In an embodiment, the ratio of PD-1 antagonist to PVRIG antagonist may be at least about 1:0.25 to at least about 1:2. In a further embodiment, the ratio of PD-1 antagonist to PVRIG antagonist may be at least about 1:0.25, at least about 1:0.30, at least about 1:0.35, at least about 1:0.40, at least about 1:0.45, at least about 1:0.50, at least about 1:0.55, at least about 1:0.60, at least about 1:0.65, at least about 1:0.70, at least about 1:0.75, at least about 1:0.80, at least about 1:0.85, at least about 1:0.90, at least about 1:0.95, at least about 1:1, at least about 1:1.05, at least about 1:1.10, at least about 1:1.15, at least about 1:1.20, at least about 1:1.25, at least about 1:1.30, at least about 1:1.35, at least about 1:1.40, at least about 1:1.45, at least about 1:1.50, at least about 1:1.55, at least about 1:1.60, at least about 1:1.65, at least about 1:1.70, at least about 1:1.75, at least about 1:1.80, at least about 1:1.85, at least about 1:1.90, at least about 1:1.95, or at least about 1.2. In yet a further embodiment, the ratio of PD-1 antagonist to PVRIG antagonist may be 1:0.25, 1:0.26, 1:0.27, 1:0.28, 1:0.29, 1:0.30, 1:0.31, 1:0.32, 1:0.33, 1:0.34, 1:0.35, 1:0.36, 1:0.37, 1:0.38, 1:0.39, 1:0.40, 1:0.41, 1:0.42, 1:0.43, 1:0.44, 1:0.45, 1:0.46, 1:0.47, 1:0.48, 1:0.49, 1:0.50, 1:0.51, 1:0.52, 1:0.53, 1:0.54, 1:0.55, 1:0.56, 1:0.57, 1:0.58, 1:0.59, 1:0.60, 1:0.61, 1:0.62, 1:0.63, 1:0.64, 1:0.65, 1:0.66, 1:0.67, 1:0.68, 1:0.69, 1:0.70, 1:0.71, 1:0.72, 1:0.73, 1:0.74, 1:0.75, 1:0.76, 1:0.77, 1:0.78, 1:0.79, 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, 1:1.20, 1:1.21, 1:1.22, 1:1.23, 1:1.24, 1:1.25, 1:1.26, 1:1.27, 1:1.28, 1:1.29, 1:1.30, 1:1.31, 1:1.32, 1:1.33, 1:1.34, 1:1.35, 1:1.36, 1:1.37, 1:1.38, 1:1.39, 1:1.40, 1:1.41, 1:1.42, 1:1.43, 1:1.44, 1:1.45, 1:1.46, 1:1.47, 1:1.48, 1:1.49, 1:1.50, 1:1.51, 1:1.52, 1:1.53, 1:1.54, 1:1.55, 1:1.56, 1:1.57, 1:1.58, 1:1.59, 1:1.60, 1:1.61, 1:1.62, 1:1.63, 1:1.64, 1:1.65, 1:1.66, 1:1.67, 1:1.68, 1:1.69, 1:1.70, 1:1.71, 1:1.72, 1:1.73, 1:1.74, 1:1.75, 1:1.76, 1:1.77, 1:1.78, 1:1.79, 1:1.80, 1:1.81, 1:1.82, 1:1.83, 1:1.84, 1:1.85, 1:1.86, 1:1.87, 1:1.88, 1:1.89, 1:1.90, 1:1.91, 1:1.92, 1:1.93, 1:1.94, 1:1.95, 1:1.96, 1:1.97, 1:1.98, 1:1.99, or 1:2.

In an embodiment, the ratio of PD-1 antagonist to CD96 antagonist may be at least about 1:0.25 to at least about 1:2. In a further embodiment, the ratio of PD-1 antagonist to CD96 antagonist may be at least about 1:0.25, at least about 1:0.30, at least about 1:0.35, at least about 1:0.40, at least about 1:0.45, at least about 1:0.50, at least about 1:0.55, at least about 1:0.60, at least about 1:0.65, at least about 1:0.70, at least about 1:0.75, at least about 1:0.80, at least about 1:0.85, at least about 1:0.90, at least about 1:0.95, at least about 1:1, at least about 1:1.05, at least about 1:1.10, at least about 1:1.15, at least about 1:1.20, at least about 1:1.25, at least about 1:1.30, at least about 1:1.35, at least about 1:1.40, at least about 1:1.45, at least about 1:1.50, at least about 1:1.55, at least about 1:1.60, at least about 1:1.65, at least about 1:1.70, at least about 1:1.75, at least about 1:1.80, at least about 1:1.85, at least about 1:1.90, at least about 1:1.95, or at least about 1.2. In yet a further embodiment, the ratio of PD-1 antagonist to CD96 antagonist may be 1:0.25, 1:0.26, 1:0.27, 1:0.28, 1:0.29, 1:0.30, 1:0.31, 1:0.32, 1:0.33, 1:0.34, 1:0.35, 1:0.36, 1:0.37, 1:0.38, 1:0.39, 1:0.40, 1:0.41, 1:0.42, 1:0.43, 1:0.44, 1:0.45, 1:0.46, 1:0.47, 1:0.48, 1:0.49, 1:0.50, 1:0.51, 1:0.52, 1:0.53, 1:0.54, 1:0.55, 1:0.56, 1:0.57, 1:0.58, 1:0.59, 1:0.60, 1:0.61, 1:0.62, 1:0.63, 1:0.64, 1:0.65, 1:0.66, 1:0.67, 1:0.68, 1:0.69, 1:0.70, 1:0.71, 1:0.72, 1:0.73, 1:0.74, 1:0.75, 1:0.76, 1:0.77, 1:0.78, 1:0.79, 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, 1:1.20, 1:1.21, 1:1.22, 1:1.23, 1:1.24, 1:1.25, 1:1.26, 1:1.27, 1:1.28, 1:1.29, 1:1.30, 1:1.31, 1:1.32, 1:1.33, 1:1.34, 1:1.35, 1:1.36, 1:1.37, 1:1.38, 1:1.39, 1:1.40, 1:1.41, 1:1.42, 1:1.43, 1:1.44, 1:1.45, 1:1.46, 1:1.47, 1:1.48, 1:1.49, 1:1.50, 1:1.51, 1:1.52, 1:1.53, 1:1.54, 1:1.55, 1:1.56, 1:1.57, 1:1.58, 1:1.59, 1:1.60, 1:1.61, 1:1.62, 1:1.63, 1:1.64, 1:1.65, 1:1.66, 1:1.67, 1:1.68, 1:1.69, 1:1.70, 1:1.71, 1:1.72, 1:1.73, 1:1.74, 1:1.75, 1:1.76, 1:1.77, 1:1.78, 1:1.79, 1:1.80, 1:1.81, 1:1.82, 1:1.83, 1:1.84, 1:1.85, 1:1.86, 1:1.87, 1:1.88, 1:1.89, 1:1.90, 1:1.91, 1:1.92, 1:1.93, 1:1.94, 1:1.95, 1:1.96, 1:1.97, 1:1.98, 1:1.99, or 1:2.

In an embodiment, the ratio of TIGIT antagonist to PVRIG antagonist may be at least about 1:0.25 to at least about 1:2. In a further embodiment, the ratio of TIGIT antagonist to PVRIG antagonist may be at least about 1:0.25, at least about 1:0.30, at least about 1:0.35, at least about 1:0.40, at least about 1:0.45, at least about 1:0.50, at least about 1:0.55, at least about 1:0.60, at least about 1:0.65, at least about 1:0.70, at least about 1:0.75, at least about 1:0.80, at least about 1:0.85, at least about 1:0.90, at least about 1:0.95, at least about 1:1, at least about 1:1.05, at least about 1:1.10, at least about 1:1.15, at least about 1:1.20, at least about 1:1.25, at least about 1:1.30, at least about 1:1.35, at least about 1:1.40, at least about 1:1.45, at least about 1:1.50, at least about 1:1.55, at least about 1:1.60, at least about 1:1.65, at least about 1:1.70, at least about 1:1.75, at least about 1:1.80, at least about 1:1.85, at least about 1:1.90, at least about 1:1.95, or at least about 1.2. In yet a further embodiment, the ratio of TIGIT antagonist to PVRIG antagonist may be 1:0.25, 1:0.26, 1:0.27, 1:0.28, 1:0.29, 1:0.30, 1:0.31, 1:0.32, 1:0.33, 1:0.34, 1:0.35, 1:0.36, 1:0.37, 1:0.38, 1:0.39, 1:0.40, 1:0.41, 1:0.42, 1:0.43, 1:0.44, 1:0.45, 1:0.46, 1:0.47, 1:0.48, 1:0.49, 1:0.50, 1:0.51, 1:0.52, 1:0.53, 1:0.54, 1:0.55, 1:0.56, 1:0.57, 1:0.58, 1:0.59, 1:0.60, 1:0.61, 1:0.62, 1:0.63, 1:0.64, 1:0.65, 1:0.66, 1:0.67, 1:0.68, 1:0.69, 1:0.70, 1:0.71, 1:0.72, 1:0.73, 1:0.74, 1:0.75, 1:0.76, 1:0.77, 1:0.78, 1:0.79, 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, 1:1.20, 1:1.21, 1:1.22, 1:1.23, 1:1.24, 1:1.25, 1:1.26, 1:1.27, 1:1.28, 1:1.29, 1:1.30, 1:1.31, 1:1.32, 1:1.33, 1:1.34, 1:1.35, 1:1.36, 1:1.37, 1:1.38, 1:1.39, 1:1.40, 1:1.41, 1:1.42, 1:1.43, 1:1.44, 1:1.45, 1:1.46, 1:1.47, 1:1.48, 1:1.49, 1:1.50, 1:1.51, 1:1.52, 1:1.53, 1:1.54, 1:1.55, 1:1.56, 1:1.57, 1:1.58, 1:1.59, 1:1.60, 1:1.61, 1:1.62, 1:1.63, 1:1.64, 1:1.65, 1:1.66, 1:1.67, 1:1.68, 1:1.69, 1:1.70, 1:1.71, 1:1.72, 1:1.73, 1:1.74, 1:1.75, 1:1.76, 1:1.77, 1:1.78, 1:1.79, 1:1.80, 1:1.81, 1:1.82, 1:1.83, 1:1.84, 1:1.85, 1:1.86, 1:1.87, 1:1.88, 1:1.89, 1:1.90, 1:1.91, 1:1.92, 1:1.93, 1:1.94, 1:1.95, 1:1.96, 1:1.97, 1:1.98, 1:1.99, or 1:2.

In an embodiment, the ratio of TIGIT antagonist to CD96 antagonist may be at least about 1:0.25 to at least about 1:2. In a further embodiment, the ratio of TIGIT antagonist to CD96 antagonist may be at least about 1:0.25, at least about 1:0.30, at least about 1:0.35, at least about 1:0.40, at least about 1:0.45, at least about 1:0.50, at least about 1:0.55, at least about 1:0.60, at least about 1:0.65, at least about 1:0.70, at least about 1:0.75, at least about 1:0.80, at least about 1:0.85, at least about 1:0.90, at least about 1:0.95, at least about 1:1, at least about 1:1.05, at least about 1:1.10, at least about 1:1.15, at least about 1:1.20, at least about 1:1.25, at least about 1:1.30, at least about 1:1.35, at least about 1:1.40, at least about 1:1.45, at least about 1:1.50, at least about 1:1.55, at least about 1:1.60, at least about 1:1.65, at least about 1:1.70, at least about 1:1.75, at least about 1:1.80, at least about 1:1.85, at least about 1:1.90, at least about 1:1.95, or at least about 1.2. In yet a further embodiment, the ratio of TIGIT antagonist to CD96 antagonist may be 1:0.25, 1:0.26, 1:0.27, 1:0.28, 1:0.29, 1:0.30, 1:0.31, 1:0.32, 1:0.33, 1:0.34, 1:0.35, 1:0.36, 1:0.37, 1:0.38, 1:0.39, 1:0.40, 1:0.41, 1:0.42, 1:0.43, 1:0.44, 1:0.45, 1:0.46, 1:0.47, 1:0.48, 1:0.49, 1:0.50, 1:0.51, 1:0.52, 1:0.53, 1:0.54, 1:0.55, 1:0.56, 1:0.57, 1:0.58, 1:0.59, 1:0.60, 1:0.61, 1:0.62, 1:0.63, 1:0.64, 1:0.65, 1:0.66, 1:0.67, 1:0.68, 1:0.69, 1:0.70, 1:0.71, 1:0.72, 1:0.73, 1:0.74, 1:0.75, 1:0.76, 1:0.77, 1:0.78, 1:0.79, 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, 1:1.20, 1:1.21, 1:1.22, 1:1.23, 1:1.24, 1:1.25, 1:1.26, 1:1.27, 1:1.28, 1:1.29, 1:1.30, 1:1.31, 1:1.32, 1:1.33, 1:1.34, 1:1.35, 1:1.36, 1:1.37, 1:1.38, 1:1.39, 1:1.40, 1:1.41, 1:1.42, 1:1.43, 1:1.44, 1:1.45, 1:1.46, 1:1.47, 1:1.48, 1:1.49, 1:1.50, 1:1.51, 1:1.52, 1:1.53, 1:1.54, 1:1.55, 1:1.56, 1:1.57, 1:1.58, 1:1.59, 1:1.60, 1:1.61, 1:1.62, 1:1.63, 1:1.64, 1:1.65, 1:1.66, 1:1.67, 1:1.68, 1:1.69, 1:1.70, 1:1.71, 1:1.72, 1:1.73, 1:1.74, 1:1.75, 1:1.76, 1:1.77, 1:1.78, 1:1.79, 1:1.80, 1:1.81, 1:1.82, 1:1.83, 1:1.84, 1:1.85, 1:1.86, 1:1.87, 1:1.88, 1:1.89, 1:1.90, 1:1.91, 1:1.92, 1:1.93, 1:1.94, 1:1.95, 1:1.96, 1:1.97, 1:1.98, 1:1.99, or 1:2.

In an embodiment, the ratio of PVRIG antagonist to CD96 antagonist may be at least about 1:0.25 to at least about 1:2. In a further embodiment, the ratio of PVRIG antagonist to CD96 antagonist may be at least about 1:0.25, at least about 1:0.30, at least about 1:0.35, at least about 1:0.40, at least about 1:0.45, at least about 1:0.50, at least about 1:0.55, at least about 1:0.60, at least about 1:0.65, at least about 1:0.70, at least about 1:0.75, at least about 1:0.80, at least about 1:0.85, at least about 1:0.90, at least about 1:0.95, at least about 1:1, at least about 1:1.05, at least about 1:1.10, at least about 1:1.15, at least about 1:1.20, at least about 1:1.25, at least about 1:1.30, at least about 1:1.35, at least about 1:1.40, at least about 1:1.45, at least about 1:1.50, at least about 1:1.55, at least about 1:1.60, at least about 1:1.65, at least about 1:1.70, at least about 1:1.75, at least about 1:1.80, at least about 1:1.85, at least about 1:1.90, at least about 1:1.95, or at least about 1.2. In yet a further embodiment, the ratio of PVRIG antagonist to CD96 antagonist may be 1:0.25, 1:0.26, 1:0.27, 1:0.28, 1:0.29, 1:0.30, 1:0.31, 1:0.32, 1:0.33, 1:0.34, 1:0.35, 1:0.36, 1:0.37, 1:0.38, 1:0.39, 1:0.40, 1:0.41, 1:0.42, 1:0.43, 1:0.44, 1:0.45, 1:0.46, 1:0.47, 1:0.48, 1:0.49, 1:0.50, 1:0.51, 1:0.52, 1:0.53, 1:0.54, 1:0.55, 1:0.56, 1:0.57, 1:0.58, 1:0.59, 1:0.60, 1:0.61, 1:0.62, 1:0.63, 1:0.64, 1:0.65, 1:0.66, 1:0.67, 1:0.68, 1:0.69, 1:0.70, 1:0.71, 1:0.72, 1:0.73, 1:0.74, 1:0.75, 1:0.76, 1:0.77, 1:0.78, 1:0.79, 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, 1:1.20, 1:1.21, 1:1.22, 1:1.23, 1:1.24, 1:1.25, 1:1.26, 1:1.27, 1:1.28, 1:1.29, 1:1.30, 1:1.31, 1:1.32, 1:1.33, 1:1.34, 1:1.35, 1:1.36, 1:1.37, 1:1.38, 1:1.39, 1:1.40, 1:1.41, 1:1.42, 1:1.43, 1:1.44, 1:1.45, 1:1.46, 1:1.47, 1:1.48, 1:1.49, 1:1.50, 1:1.51, 1:1.52, 1:1.53, 1:1.54, 1:1.55, 1:1.56, 1:1.57, 1:1.58, 1:1.59, 1:1.60, 1:1.61, 1:1.62, 1:1.63, 1:1.64, 1:1.65, 1:1.66, 1:1.67, 1:1.68, 1:1.69, 1:1.70, 1:1.71, 1:1.72, 1:1.73, 1:1.74, 1:1.75, 1:1.76, 1:1.77, 1:1.78, 1:1.79, 1:1.80, 1:1.81, 1:1.82, 1:1.83, 1:1.84, 1:1.85, 1:1.86, 1:1.87, 1:1.88, 1:1.89, 1:1.90, 1:1.91, 1:1.92, 1:1.93, 1:1.94, 1:1.95, 1:1.96, 1:1.97, 1:1.98, 1:1.99, or 1:2.

In an embodiment, a combination may comprise a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a combination may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a combination may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a combination may comprise a PD-1 antagonist and a TIGIT antagonist wherein the PD-1 antagonist is dostarlimab and the TIGIT antagonist is vibostolimab. In an embodiment, a combination may comprise a a PD-1 antagonist and a TIGIT antagonist wherein the PD-1 antagonist is dostarlimab and the TIGIT antagonist comprises an anti-TIGIT antibody comprising a heavy chain and a light chain variable sequence as shown in Table X.

In an embodiment, a combination may comprise a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a combination may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a combination may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, a combination may comprise a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a combination may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a combination may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, a combination may comprise a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a TIGIT antagonist and a PVRIG antagonist wherein the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a combination may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, a combination may comprise a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a combination may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a combination may comprise a TIGIT antagonist and a CD96 antagonist wherein the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a combination may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, a combination may comprise a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination may comprise a PVRIG antagonist and a CD96 antagonist wherein the PRVIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In another embodiment, a combination may comprise a PD1 antagonist, a TIGIT antagonist, and a PVRIG antagonist. In another embodiment, a combination may comprise a PD1 antagonist, a TIGIT antagonist, and a CD96 antagonist. In another embodiment, a combination may comprise a PD1 antagonist, a PVRIG antagonist, and a CD96 antagonist. In another embodiment, a combination may comprise a TIGIT antagonist, a PVRIG antagonist, and a CD96 antagonist.

In an embodiment, the ratio of PD-1 antagonist to TIGIT antagonist to PVRIG antagonist may be at least about 1:0.25:0.25 to at least about 1:2:2. In a further embodiment, the ratio of PD-1 antagonist to TIGIT antagonist to PVRIG antagonist may be at least about 1:0.25:0.25, at least about 1:0.30:0.30, at least about 1:0.35:0.35, at least about 1:0.40:0.40, at least about 1:0.45:0.45, at least about 1:0.50:0.50, at least about 1:0.55:0.55, at least about 1:0.60:0.60, at least about 1:0.65:0.65, at least about 1:0.70:0.70, at least about 1:0.75:0.75, at least about 1:0.80:0.80, at least about 1:0.85:0.85, at least about 1:0.90:0.90, at least about 1:0.95:0.95, at least about 1:1:1, at least about 1:1.05:1.05, at least about 1:1.10:1.10, at least about 1:1.15:1.15, at least about 1:1.20:1.20, at least about 1:1.25:1.25, at least about 1:1.30:1.30, at least about 1:1.35:1.35, at least about 1:1.40:1.40, at least about 1:1.45:1.45, at least about 1:1.50:1.50, at least about 1:1.55:1.55, at least about 1:1.60:1.60, at least about 1:1.65:1.65, at least about 1:1.70:1.70, at least about 1:1.75:1.75, at least about 1:1.80:1.80, at least about 1:1.85:1.85, at least about 1:1.90:1.90, at least about 1:1.95:1.95, or at least about 1:2:2.

In an embodiment, the ratio of PD-1 antagonist to TIGIT antagonist to CD96 antagonist may be at least about 1:0.25:0.25 to at least about 1:2:2. In a further embodiment, the ratio of PD-1 antagonist to TIGIT antagonist to CD96 antagonist may be at least about 1:0.25:0.25, at least about 1:0.30:0.30, at least about 1:0.35:0.35, at least about 1:0.40:0.40, at least about 1:0.45:0.45, at least about 1:0.50:0.50, at least about 1:0.55:0.55, at least about 1:0.60:0.60, at least about 1:0.65:0.65, at least about 1:0.70:0.70, at least about 1:0.75:0.75, at least about 1:0.80:0.80, at least about 1:0.85:0.85, at least about 1:0.90:0.90, at least about 1:0.95:0.95, at least about 1:1:1, at least about 1:1.05:1.05, at least about 1:1.10:1.10, at least about 1:1.15:1.15, at least about 1:1.20:1.20, at least about 1:1.25:1.25, at least about 1:1.30:1.30, at least about 1:1.35:1.35, at least about 1:1.40:1.40, at least about 1:1.45:1.45, at least about 1:1.50:1.50, at least about 1:1.55:1.55, at least about 1:1.60:1.60, at least about 1:1.65:1.65, at least about 1:1.70:1.70, at least about 1:1.75:1.75, at least about 1:1.80:1.80, at least about 1:1.85:1.85, at least about 1:1.90:1.90, at least about 1:1.95:1.95, or at least about 1:2:2.

In an embodiment, the ratio of PD-1 antagonist to PVRIG antagonist to CD96 antagonist may be at least about 1:0.25:0.25 to at least about 1:2:2. In a further embodiment, the ratio of PD-1 antagonist to PVRIG antagonist to CD96 antagonist may be at least about 1:0.25:0.25, at least about 1:0.30:0.30, at least about 1:0.35:0.35, at least about 1:0.40:0.40, at least about 1:0.45:0.45, at least about 1:0.50:0.50, at least about 1:0.55:0.55, at least about 1:0.60:0.60, at least about 1:0.65:0.65, at least about 1:0.70:0.70, at least about 1:0.75:0.75, at least about 1:0.80:0.80, at least about 1:0.85:0.85, at least about 1:0.90:0.90, at least about 1:0.95:0.95, at least about 1:1:1, at least about 1:1.05:1.05, at least about 1:1.10:1.10, at least about 1:1.15:1.15, at least about 1:1.20:1.20, at least about 1:1.25:1.25, at least about 1:1.30:1.30, at least about 1:1.35:1.35, at least about 1:1.40:1.40, at least about 1:1.45:1.45, at least about 1:1.50:1.50, at least about 1:1.55:1.55, at least about 1:1.60:1.60, at least about 1:1.65:1.65, at least about 1:1.70:1.70, at least about 1:1.75:1.75, at least about 1:1.80:1.80, at least about 1:1.85:1.85, at least about 1:1.90:1.90, at least about 1:1.95:1.95, or at least about 1:2:2.

In an embodiment, the ratio of TIGIT antagonist to PVRIG antagonist to CD96 antagonist may be at least about 1:0.25:0.25 to at least about 1:2:2. In a further embodiment, the ratio of TIGIT antagonist to PVRIG antagonist to CD96 antagonist may be at least about 1:0.25:0.25, at least about 1:0.30:0.30, at least about 1:0.35:0.35, at least about 1:0.40:0.40, at least about 1:0.45:0.45, at least about 1:0.50:0.50, at least about 1:0.55:0.55, at least about 1:0.60:0.60, at least about 1:0.65:0.65, at least about 1:0.70:0.70, at least about 1:0.75:0.75, at least about 1:0.80:0.80, at least about 1:0.85:0.85, at least about 1:0.90:0.90, at least about 1:0.95:0.95, at least about 1:1:1, at least about 1:1.05:1.05, at least about 1:1.10:1.10, at least about 1:1.15:1.15, at least about 1:1.20:1.20, at least about 1:1.25:1.25, at least about 1:1.30:1.30, at least about 1:1.35:1.35, at least about 1:1.40:1.40, at least about 1:1.45:1.45, at least about 1:1.50:1.50, at least about 1:1.55:1.55, at least about 1:1.60:1.60, at least about 1:1.65:1.65, at least about 1:1.70:1.70, at least about 1:1.75:1.75, at least about 1:1.80:1.80, at least about 1:1.85:1.85, at least about 1:1.90:1.90, at least about 1:1.95:1.95, or at least about 1:2:2. In an embodiment, a combination may comprise a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist is dostarlimab, the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, a combination may comprise a PD-1 antagonist, a TIGIT antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a combination may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a combination may comprise a PD-1 antagonist, a TIGIT antagonist, and a CD96 antagonist wherein the PD-1 antagonist is dostarlimab, the TIGIT antagonist comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a combination may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, a combination may comprise a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination may comprise a PD-1 antagonist, a PVRIG antagonist, and a CD96 antagonist wherein the PD-1 antagonist comprises HC comprising SEQ ID NO: 160 and LC comprising SEQ ID NO: 161, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination may comprise a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ antagonist, and a CD96 antagonist wherein the TIGIT antagonist comprises VH comprising comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In a further embodiment, the ratio of PD-1 antagonist to TIGIT antagonist to PVRIG antagonist may be at least about 1:0.25:0.25, at least about 1:0.30:0.30, at least about 1:0.35:0.35, at least about 1:0.40:0.40, at least about 1:0.45:0.45, at least about 1:0.50:0.50, at least about 1:0.55:0.55, at least about 1:0.60:0.60, at least about 1:0.65:0.65, at least about 1:0.70:0.70, at least about 1:0.75:0.75, at least about 1:0.80:0.80, at least about 1:0.85:0.85, at least about 1:0.90:0.90, at least about 1:0.95:0.95, at least about 1:1:1, at least about 1:1.05:1.05, at least about 1:1.10:1.10, at least about 1:1.15:1.15, at least about 1:1.20:1.20, at least about 1:1.25:1.25, at least about 1:1.30:1.30, at least about 1:1.35:1.35, at least about 1:1.40:1.40, at least about 1:1.45:1.45, at least about 1:1.50:1.50, at least about 1:1.55:1.55, at least about 1:1.60:1.60, at least about 1:1.65:1.65, at least about 1:1.70:1.70, at least about 1:1.75:1.75, at least about 1:1.80:1.80, at least about 1:1.85:1.85, at least about 1:1.90:1.90, at least about 1:1.95:1.95, or at least about 1:2:2.

In a further embodiment, the ratio of PD-1 antagonist to TIGIT antagonist to PVRIG antagonist may be at least about 1:0.25:0.30, at least about 1:0.25:0.35, at least about 1:0.25:0.40, at least about 1:0.25:0.45, at least about 1:0.25:0.50, at least about 1:0.25:0.55, at least about 1:0.25:0.60, at least about 1:0.25:0.65, at least about 1:0.25:0.70, at least about 1:0.25:0.75, at least about 1:0.25:0.80, at least about 1:0.25:0.85, at least about 1:0.25:0.90, at least about 1:0.25:0.95, at least about 1:0.25:1, at least about 1:0.25:1.05, at least about 1:0.25:1.10, at least about 1:0.25:1.15, at least about 1:0.25:1.20, at least about 1:0.25:1.25, at least about 1:0.25:1.30, at least about 1:0.25:1.35, at least about 1:0.25:1.40, at least about 1:0.25:1.45, at least about 1:0.25:1.50, at least about 1:0.25:1.55, at least about 1:0.25:1.60, at least about 1:0.25:1.65, at least about 1:0.25:1.70, at least about 1:0.25:1.75, at least about 1:0.25:1.80, at least about 1:0.25:1.85, at least about 1:0.25:1.90, at least about 1:0.25:1.95, or at least about 1:0.25:2.

In a further embodiment, the ratio of PD-1 antagonist to TIGIT antagonist to PVRIG antagonist may be at least about 1:0.30:0.25, at least about 1:0.30:0.30, at least about 1:0.30:0.35, at least about 1:0.30:0.40, at least about 1:0.30:0.45, at least about 1:0.30:0.50, at least about 1:0.30:0.55, at least about 1:0.30:0.60, at least about 1:0.30:0.65, at least about 1:0.30:0.70, at least about 1:0.30:0.75, at least about 1:0.30:0.80, at least about 1:0.30:0.85, at least about 1:0.30:0.90, at least about 1:0.30:0.95, at least about 1:0.30:1, at least about 1:0.30:1.05, at least about 1:0.30:1.10, at least about 1:0.30:1.15, at least about 1:0.30:1.20, at least about 1:0.30:1.25, at least about 1:0.30:1.30, at least about 1:0.30:1.35, at least about 1:0.30:1.40, at least about 1:0.30:1.45, at least about 1:0.30:1.50, at least about 1:0.30:1.55, at least about 1:0.30:1.60, at least about 1:0.30:1.65, at least about 1:0.30:1.70, at least about 1:0.30:1.75, at least about 1:0.30:1.80, at least about 1:0.30:1.85, at least about 1:0.30:1.90, at least about 1:0.30:1.95, or at least about 1:0.30:2.

In a further embodiment, the ratio of PD-1 antagonist to TIGIT antagonist to PVRIG antagonist may be at least about 1:0.35:0.25, at least about 1:0.35:0.30, at least about 1:0.35:0.35, at least about 1:0.35:0.40, at least about 1:0.35:0.45, at least about 1:0.35:0.50, at least about 1:0.35:0.55, at least about 1:0.35:0.60, at least about 1:0.35:0.65, at least about 1:0.35:0.70, at least about 1:0.35:0.75, at least about 1:0.35:0.80, at least about 1:0.35:0.85, at least about 1:0.35:0.90, at least about 1:0.35:0.95, at least about 1:0.35:1, at least about 1:0.35:1.05, at least about 1:0.35:1.10, at least about 1:0.35:1.15, at least about 1:0.35:1.20, at least about 1:0.35:1.25, at least about 1:0.35:1.30, at least about 1:0.35:1.35, at least about 1:0.35:1.40, at least about 1:0.35:1.45, at least about 1:0.35:1.50, at least about 1:0.35:1.55, at least about 1:0.35:1.60, at least about 1:0.35:1.65, at least about 1:0.35:1.70, at least about 1:0.35:1.75, at least about 1:0.35:1.80, at least about 1:0.35:1.85, at least about 1:0.35:1.90, at least about 1:0.35:1.95, or at least about 1:0.35:2.

In a further embodiment, the ratio of PD-1 antagonist to TIGIT antagonist to PVRIG antagonist may be at least about 1:0.40:0.25, at least about 1:0.40:0.30, at least about 1:0.40:0.35, at least about 1:0.40:0.40, at least about 1:0.40:0.45, at least about 1:0.40:0.50, at least about 1:0.40:0.55, at least about 1:0.40:0.60, at least about 1:0.40:0.65, at least about 1:0.40:0.70, at least about 1:0.40:0.75, at least about 1:0.40:0.80, at least about 1:0.40:0.85, at least about 1:0.40:0.90, at least about 1:0.40:0.95, at least about 1:0.40:1, at least about 1:0.40:1.05, at least about 1:0.40:1.10, at least about 1:0.40:1.15, at least about 1:0.40:1.20, at least about 1:0.40:1.25, at least about 1:0.40:1.30, at least about 1:0.40:1.35, at least about 1:0.40:1.40, at least about 1:0.40:1.45, at least about 1:0.40:1.50, at least about 1:0.40:1.55, at least about 1:0.40:1.60, at least about 1:0.40:1.65, at least about 1:0.40:1.70, at least about 1:0.40:1.75, at least about 1:0.40:1.80, at least about 1:0.40:1.85, at least about 1:0.40:1.90, at least about 1:0.40:1.95, or at least about 1:0.40:2.

In a further embodiment, the ratio of PD-1 antagonist to TIGIT antagonist to PVRIG antagonist may be at least about 1:0.45:0.25, at least about 1:0.45:0.30, at least about 1:0.45:0.35, at least about 1:0.45:0.40, at least about 1:0.45:0.45, at least about 1:0.45:0.50, at least about 1:0.45:0.55, at least about 1:0.45:0.60, at least about 1:0.45:0.65, at least about 1:0.45:0.70, at least about 1:0.45:0.75, at least about 1:0.45:0.80, at least about 1:0.45:0.85, at least about 1:0.45:0.90, at least about 1:0.45:0.95, at least about 1:0.45:1, at least about 1:0.45:1.05, at least about 1:0.45:1.10, at least about 1:0.45:1.15, at least about 1:0.45:1.20, at least about 1:0.45:1.25, at least about 1:0.45:1.30, at least about 1:0.45:1.35, at least about 1:0.45:1.40, at least about 1:0.45:1.45, at least about 1:0.45:1.50, at least about 1:0.45:1.55, at least about 1:0.45:1.60, at least about 1:0.45:1.65, at least about 1:0.45:1.70, at least about 1:0.45:1.75, at least about 1:0.45:1.80, at least about 1:0.45:1.85, at least about 1:0.45:1.90, at least about 1:0.45:1.95, or at least about 1:0.45:2.

In a further embodiment, the ratio of PD-1 antagonist to TIGIT antagonist to PVRIG antagonist may be at least about 1:0.50:0.25, at least about 1:0.50:0.30, at least about 1:0.50:0.35, at least about 1:0.50:0.40, at least about 1:0.50:0.45, at least about 1:0.50:0.50, at least about 1:0.50:0.55, at least about 1:0.50:0.60, at least about 1:0.50:0.65, at least about 1:0.50:0.70, at least about 1:0.50:0.75, at least about 1:0.50:0.80, at least about 1:0.50:0.85, at least about 1:0.50:0.90, at least about 1:0.50:0.95, at least about 1:0.50:1, at least about 1:0.50:1.05, at least about 1:0.50:1.10, at least about 1:0.50:1.15, at least about 1:0.50:1.20, at least about 1:0.50:1.25, at least about 1:0.50:1.30, at least about 1:0.50:1.35, at least about 1:0.50:1.40, at least about 1:0.50:1.45, at least about 1:0.50:1.50, at least about 1:0.50:1.55, at least about 1:0.50:1.60, at least about 1:0.50:1.65, at least about 1:0.50:1.70, at least about 1:0.50:1.75, at least about 1:0.50:1.80, at least about 1:0.50:1.85, at least about 1:0.50:1.90, at least about 1:0.50:1.95, or at least about 1:0.50:2.

In a further embodiment, the ratio of PD-1 antagonist to TIGIT antagonist to PVRIG antagonist may be at least about 1:0.55:0.25, at least about 1:0.55:0.30, at least about 1:0.55:0.35, at least about 1:0.55:0.40, at least about 1:0.55:0.45, at least about 1:0.55:0.50, at least about 1:0.55:0.55, at least about 1:0.55:0.60, at least about 1:0.55:0.65, at least about 1:0.55:0.70, at least about 1:0.55:0.75, at least about 1:0.55:0.80, at least about 1:0.55:0.85, at least about 1:0.55:0.90, at least about 1:0.55:0.95, at least about 1:0.55:1, at least about 1:0.55:1.05, at least about 1:0.55:1.10, at least about 1:0.55:1.15, at least about 1:0.55:1.20, at least about 1:0.55:1.25, at least about 1:0.55:1.30, at least about 1:0.55:1.35, at least about 1:0.55:1.40, at least about 1:0.55:1.45, at least about 1:0.55:1.50, at least about 1:0.55:1.55, at least about 1:0.55:1.60, at least about 1:0.55:1.65, at least about 1:0.55:1.70, at least about 1:0.55:1.75, at least about 1:0.55:1.80, at least about 1:0.55:1.85, at least about 1:0.55:1.90, 1:0.55:1.95, or at least about 1:0.55:2.

In a further embodiment, the ratio of PD-1 antagonist to TIGIT antagonist to PVRIG antagonist may be at least about 1:0.60:0.25, at least about 1:0.60:0.30, at least about 1:0.60:0.35, at least about 1:0.60:0.40, at least about 1:0.60:0.45, at least about 1:0.60:0.50, at least about 1:0.60:0.55, at least about 1:0.60:0.60, at least about 1:0.60:0.65, at least about 1:0.60:0.70, at least about 1:0.60:0.75, at least about 1:0.60:0.80, at least about 1:0.60:0.85, at least about 1:0.60:0.90, at least about 1:0.60:0.95, at least about 1:0.60:1, at least about 1:0.60:1.05, at least about 1:0.60:1.10, at least about 1:0.60:1.15, at least about 1:0.60:1.20, at least about 1:0.60:1.25, at least about 1:0.60:1.30, at least about 1:0.60:1.35, at least about 1:0.60:1.40, at least about 1:0.60:1.45, at least about 1:0.60:1.50, at least about 1:0.60:1.55, at least about 1:0.60:1.60, at least about 1:0.60:1.65, at least about 1:0.60:1.70, at least about 1:0.60:1.75, at least about 1:0.60:1.80, at least about 1:0.60:1.85, at least about 1:0.60:1.90, at least about 1:0.60:1.95, or at least about 1:0.60:2.

In a further embodiment, the ratio of PD-1 antagonist to TIGIT antagonist to PVRIG antagonist may be at least about 1:0.65:0.25, at least about 1:0.65:0.30, at least about 1:0.65:0.35, at least about 1:0.65:0.40, at least about 1:0.65:0.45, at least about 1:0.65:0.50, at least about 1:0.65:0.55, at least about 1:0.65:0.60, at least about 1:0.65:0.65, at least about 1:0.65:0.70, at least about 1:0.65:0.75, at least about 1:0.65:0.80, at least about 1:0.65:0.85, at least about 1:0.65:0.90, at least about 1:0.65:0.95, at least about 1:0.65:1, at least about 1:0.65:1.05, at least about 1:0.65:1.10, at least about 1:0.65:1.15, at least about 1:0.65:1.20, at least about 1:0.65:1.25, at least about 1:0.65:1.30, at least about 1:0.65:1.35, at least about 1:0.65:1.40, at least about 1:0.65:1.45, at least about 1:0.65:1.50, at least about 1:0.65:1.55, at least about 1:0.65:1.60, at least about 1:0.65:1.65, at least about 1:0.65:1.70, at least about 1:0.65:1.75, at least about 1:0.65:1.80, at least about 1:0.65:1.85, at least about 1:0.65:1.90, at least about 1:0.65:1.95, or at least about 1:0.65:2.

In a further embodiment, the ratio of PD-1 antagonist to TIGIT antagonist to PVRIG antagonist may be at least about 1:0.70:0.25, at least about 1:0.70:0.30, at least about 1:0.70:0.35, at least about 1:0.70:0.40, at least about 1:0.70:0.45, at least about 1:0.70:0.50, at least about 1:0.70:0.55, at least about 1:0.70:0.60, at least about 1:0.70:0.65, at least about 1:0.70:0.70, at least about 1:0.70:0.75, at least about 1:0.70:0.80, at least about 1:0.70:0.85, at least about 1:0.70:0.90, at least about 1:0.70:0.95, at least about 1:0.70:1, at least about 1:0.70:1.05, at least about 1:0.70:1.10, at least about 1:0.70:1.15, at least about 1:0.70:1.20, at least about 1:0.70:1.25, at least about 1:0.70:1.30, at least about 1:0.70:1.35, at least about 1:0.70:1.40, at least about 1:0.70:1.45, at least about 1:0.70:1.50, at least about 1:0.70:1.55, at least about 1:0.70:1.60, at least about 1:0.70:1.65, at least about 1:0.70:1.70, at least about 1:0.70:1.75, at least about 1:0.70:1.80, at least about 1:0.70:1.85, at least about 1:0.70:1.90, at least about 1:0.70:1.95, or at least about 1:0.70:2.

In a further embodiment, the ratio of PD-1 antagonist to TIGIT antagonist to PVRIG antagonist may be at least about 1:0.75:0.25, at least about 1:0.75:0.30, at least about 1:0.75:0.35, at least about 1:0.75:0.40, at least about 1:0.75:0.45, at least about 1:0.75:0.50, at least about 1:0.75:0.55, at least about 1:0.75:0.60, at least about 1:0.75:0.65, at least about 1:0.75:0.70, at least about 1:0.75:0.75, at least about 1:0.75:0.80, at least about 1:0.75:0.85, at least about 1:0.75:0.90, at least about 1:0.75:0.95, at least about 1:0.75:1, at least about 1:0.75:1.05, at least about 1:0.75:1.10, at least about 1:0.75:1.15, at least about 1:0.75:1.20, at least about 1:0.75:1.25, at least about 1:0.75:1.30, at least about 1:0.75:1.35, at least about 1:0.75:1.40, at least about 1:0.75:1.45, at least about 1:0.75:1.50, at least about 1:0.75:1.55, at least about 1:0.75:1.60, at least about 1:0.75:1.65, at least about 1:0.75:1.70, at least about 1:0.75:1.75, at least about 1:0.75:1.80, at least about 1:0.75:1.85, at least about 1:0.75:1.90, at least about 1:0.75:1.95, or at least about 1:0.75:2.

In a further embodiment, the ratio of PD-1 antagonist to TIGIT antagonist to PVRIG antagonist may be at least about 1:0.80:0.25, at least about 1:0.80:0.30, at least about 1:0.80:0.35, at least about 1:0.80:0.40, at least about 1:0.80:0.45, at least about 1:0.80:0.50, at least about 1:0.80:0.55, at least about 1:0.80:0.60, at least about 1:0.80:0.65, at least about 1:0.80:0.70, at least about 1:0.80:0.75, at least about 1:0.80:0.80, at least about 1:0.80:0.85, at least about 1:0.80:0.90, at least about 1:0.80:0.95, at least about 1:0.80:1, at least about 1:0.80:1.05, at least about 1:0.80:1.10, at least about 1:0.80:1.15, at least about 1:0.80:1.20, at least about 1:0.80:1.25, at least about 1:0.80:1.30, at least about 1:0.80:1.35, at least about 1:0.80:1.40, at least about 1:0.80:1.45, at least about 1:0.80:1.50, at least about 1:0.80:1.55, at least about 1:0.80:1.60, at least about 1:0.80:1.65, at least about 1:0.80:1.70, at least about 1:0.80:1.75, at least about 1:0.80:1.80, at least about 1:0.80:1.85, at least about 1:0.80:1.90, at least about 1:0.80:1.95, or at least about 1:0.80:2.

In a further embodiment, the ratio of PD-1 antagonist to TIGIT antagonist to PVRIG antagonist may be at least about 1:0.85:0.25, at least about 1:0.85:0.30, at least about 1:0.85:0.35, at least about 1:0.85:0.40, at least about 1:0.85:0.45, at least about 1:0.85:0.50, at least about 1:0.85:0.55, at least about 1:0.85:0.60, at least about 1:0.85:0.65, at least about 1:0.85:0.70, at least about 1:0.85:0.75, at least about 1:0.85:0.80, at least about 1:0.85:0.85, at least about 1:0.85:0.90, at least about 1:0.85:0.95, at least about 1:0.85:1, at least about 1:0.85:1.05, at least about 1:0.85:1.10, at least about 1:0.85:1.15, at least about 1:0.85:1.20, at least about 1:0.85:1.25, at least about 1:0.85:1.30, at least about 1:0.85:1.35, at least about 1:0.85:1.40, at least about 1:0.85:1.45, at least about 1:0.85:1.50, at least about 1:0.85:1.55, at least about 1:0.85:1.60, at least about 1:0.85:1.65, at least about 1:0.85:1.70, at least about 1:0.85:1.75, at least about 1:0.85:1.80, at least about 1:0.85:1.85, at least about 1:0.85:1.90, at least about 1:0.85:1.95, or at least about 1:0.85:2.

In a further embodiment, the ratio of PD-1 antagonist to TIGIT antagonist to PVRIG antagonist may be at least about 1:0.90:0.25, at least about 1:0.90:0.30, at least about 1:0.90:0.35, at least about 1:0.90:0.40, at least about 1:0.90:0.45, at least about 1:0.90:0.50, at least about 1:0.90:0.55, at least about 1:0.90:0.60, at least about 1:0.90:0.65, at least about 1:0.90:0.70, at least about 1:0.90:0.75, at least about 1:0.90:0.80, at least about 1:0.90:0.85, at least about 1:0.90:0.90, at least about 1:0.90:0.95, at least about 1:0.90:1, at least about 1:0.90:1.05, at least about 1:0.90:1.10, at least about 1:0.90:1.15, at least about 1:0.90:1.20, at least about 1:0.90:1.25, at least about 1:0.90:1.30, at least about 1:0.90:1.35, at least about 1:0.90:1.40, at least about 1:0.90:1.45, at least about 1:0.90:1.50, at least about 1:0.90:1.55, at least about 1:0.90:1.60, at least about 1:0.90:1.65, at least about 1:0.90:1.70, at least about 1:0.90:1.75, at least about 1:0.90:1.80, at least about 1:0.90:1.85, at least about 1:0.90:1.90, at least about 1:0.90:1.95, or at least about 1:0.90:2.

In a further embodiment, the ratio of PD-1 antagonist to TIGIT antagonist to PVRIG antagonist may be at least about 1:0.95:0.25, at least about 1:0.95:0.30, at least about 1:0.95:0.35, at least about 1:0.95:0.40, at least about 1:0.95:0.45, at least about 1:0.95:0.50, at least about 1:0.95:0.55, at least about 1:0.95:0.60, at least about 1:0.95:0.65, at least about 1:0.95:0.70, at least about 1:0.95:0.75, at least about 1:0.95:0.80, at least about 1:0.95:0.85, at least about 1:0.95:0.90, at least about 1:0.95:0.95, at least about 1:0.95:1, at least about 1:0.95:1.05, at least about 1:0.95:1.10, at least about 1:0.95:1.15, at least about 1:0.95:1.20, at least about 1:0.95:1.25, at least about 1:0.95:1.30, at least about 1:0.95:1.35, at least about 1:0.95:1.40, at least about 1:0.95:1.45, at least about 1:0.95:1.50, at least about 1:0.95:1.55, at least about 1:0.95:1.60, at least about 1:0.95:1.65, at least about 1:0.95:1.70, at least about 1:0.95:1.75, at least about 1:0.95:1.80, at least about 1:0.95:1.85, at least about 1:0.95:1.90, at least about 1:0.95:1.95, or at least about 1:0.95:2.

In a further embodiment, the ratio of PD-1 antagonist to TIGIT antagonist to PVRIG antagonist may be at least about 1:1:0.25, at least about 1:1:0.30, at least about 1:1:0.35, at least about 1:1:0.40, at least about 1:1:0.45, at least about 1:1:0.50, at least about 1:1:0.55, at least about 1:1:0.60, at least about 1:1:0.65, at least about 1:1:0.70, at least about 1:1:0.75, at least about 1:1:0.80, at least about 1:1:0.85, at least about 1:1:0.90, at least about 1:1:0.95, at least about 1:1:1, at least about 1:1:1.05, at least about 1:1:1.10, at least about 1:1:1.15, at least about 1:1:1.20, at least about 1:1:1.25, at least about 1:1:1.30, at least about 1:1:1.35, at least about 1:1:1.40, at least about 1:1:1.45, at least about 1:1:1.50, at least about 1:1:1.55, at least about 1:1:1.60, at least about 1:1:1.65, at least about 1:1:1.70, at least about 1:1:1.75, at least about 1:1:1.80, at least about 1:1:1.85, at least about 1:1:1.90, at least about 1:1:1.95, or at least about 1:1:2.

In a further embodiment, the ratio of PD-1 antagonist to TIGIT antagonist to PVRIG antagonist may be at least about 1:1.05:0.25, at least about 1:1.05:0.30, at least about 1:1.05:0.35, at least about 1:1.05:0.40, at least about 1:1.05:0.45, at least about 1:1.05:0.50, at least about 1:1.05:0.55, at least about 1:1.05:0.60, at least about 1:1.05:0.65, at least about 1:1.05:0.70, at least about 1:1.05:0.75, at least about 1:1.05:0.80, at least about 1:1.05:0.85, at least about 1:1.05:0.90, at least about 1:1.05:0.95, at least about 1:1.05:1, at least about 1:1.05:1.05, at least about 1:1.05:1.10, at least about 1:1.05:1.15, at least about 1:1.05:1.20, at least about 1:1.05:1.25, at least about 1:1.05:1.30, at least about 1:1.05:1.35, at least about 1:1.05:1.40, at least about 1:1.05:1.45, at least about 1:1.05:1.50, at least about 1:1.05:1.55, at least about 1:1.05:1.60, at least about 1:1.05:1.65, at least about 1:1.05:1.70, at least about 1:1.05:1.75, at least about 1:1.05:1.80, at least about 1:1.05:1.85, at least about 1:1.05:1.90, at least about 1:1.05:1.95, or at least about 1:1.05:2.

In a further embodiment, the ratio of PD-1 antagonist to TIGIT antagonist to PVRIG antagonist may be at least about 1:1.10:0.25, at least about 1:1.10:0.30, at least about 1:1.10:0.35, at least about 1:1.10:0.40, at least about 1:1.10:0.45, at least about 1:1.10:0.50, at least about 1:1.10:0.55, at least about 1:1.10:0.60, at least about 1:1.10:0.65, at least about 1:1.10:0.70, at least about 1:1.10:0.75, at least about 1:1.10:0.80, at least about 1:1.10:0.85, at least about 1:1.10:0.90, at least about 1:1.10:0.95, at least about 1:1.10:1, at least about 1:1.10:1.05, at least about 1:1.10:1.10, at least about 1:1.10:1.15, at least about 1:1.10:1.20, at least about 1:1.10:1.25, at least about 1:1.10:1.30, at least about 1:1.10:1.35, at least about 1:1.10:1.40, at least about 1:1.10:1.45, at least about 1:1.10:1.50, at least about 1:1.10:1.55, at least about 1:1.10:1.60, at least about 1:1.10:1.65, at least about 1:1.10:1.70, at least about 1:1.10:1.75, at least about 1:1.10:1.80, at least about 1:1.10:1.85, at least about 1:1.10:1.90, at least about 1:1.10:1.95, or at least about 1:1.10:2.

In a further embodiment, the ratio of PD-1 antagonist to TIGIT antagonist to PVRIG antagonist may be at least about 1:1.15:0.25, at least about 1:1.15:0.30, at least about 1:1.15:0.35, at least about 1:1.15:0.40, at least about 1:1.15:0.45, at least about 1:1.15:0.50, at least about 1:1.15:0.55, at least about 1:1.15:0.60, at least about 1:1.15:0.65, at least about 1:1.15:0.70, at least about 1:1.15:0.75, at least about 1:1.15:0.80, at least about 1:1.15:0.85, at least about 1:1.15:0.90, at least about 1:1.15:0.95, at least about 1:1.15:1, at least about 1:1.15:1.05, at least about 1:1.15:1.10, at least about 1:1.15:1.15, at least about 1:1.15:1.20, at least about 1:1.15:1.25, at least about 1:1.15:1.30, at least about 1:1.15:1.35, at least about 1:1.15:1.40, at least about 1:1.15:1.45, at least about 1:1.15:1.50, at least about 1:1.15:1.55, at least about 1:1.15:1.60, at least about 1:1.15:1.65, at least about 1:1.15:1.70, at least about 1:1.15:1.75, at least about 1:1.15:1.80, at least about 1:1.15:1.85, at least about 1:1.15:1.90, at least about 1:1.15:1.95, or at least about 1:1.15:2.

In a further embodiment, the ratio of PD-1 antagonist to TIGIT antagonist to PVRIG antagonist may be at least about 1:1.20:0.25, at least about 1:1.20:0.30, at least about 1:1.20:0.35, at least about 1:1.20:0.40, at least about 1:1.20:0.45, at least about 1:1.20:0.50, at least about 1:1.20:0.55, at least about 1:1.20:0.60, at least about 1:1.20:0.65, at least about 1:1.20:0.70, at least about 1:1.20:0.75, at least about 1:1.20:0.80, at least about 1:1.20:0.85, at least about 1:1.20:0.90, at least about 1:1.20:0.95, at least about 1:1.20:1, at least about 1:1.20:1.05, at least about 1:1.20:1.10, at least about 1:1.20:1.15, at least about 1:1.20:1.20, at least about 1:1.20:1.25, at least about 1:1.20:1.30, at least about 1:1.20:1.35, at least about 1:1.20:1.40, at least about 1:1.20:1.45, at least about 1:1.20:1.50, at least about 1:1.20:1.55, at least about 1:1.20:1.60, at least about 1:1.20:1.65, at least about 1:1.20:1.70, at least about 1:1.20:1.75, at least about 1:1.20:1.80, at least about 1:1.20:1.85, at least about 1:1.20:1.90, at least about 1:1.20:1.95, or at least about 1:1.20:2.

In a further embodiment, the ratio of PD-1 antagonist to TIGIT antagonist to PVRIG antagonist may be at least about 1:1.25:0.25, at least about 1:1.25:0.30, at least about 1:1.25:0.35, at least about 1:1.25:0.40, at least about 1:1.25:0.45, at least about 1:1.25:0.50, at least about 1:1.25:0.55, at least about 1:1.25:0.60, at least about 1:1.25:0.65, at least about 1:1.25:0.70, at least about 1:1.25:0.75, at least about 1:1.25:0.80, at least about 1:1.25:0.85, at least about 1:1.25:0.90, at least about 1:1.25:0.95, at least about 1:1.25:1, at least about 1:1.25:1.05, at least about 1:1.25:1.10, at least about 1:1.25:1.15, at least about 1:1.25:1.20, at least about 1:1.25:1.25, at least about 1:1.25:1.30, at least about 1:1.25:1.35, at least about 1:1.25:1.40, at least about 1:1.25:1.45, at least about 1:1.25:1.50, at least about 1:1.25:1.55, at least about 1:1.25:1.60, at least about 1:1.25:1.65, at least about 1:1.25:1.70, at least about 1:1.25:1.75, at least about 1:1.25:1.80, at least about 1:1.25:1.85, at least about 1:1.25:1.90, at least about 1:1.25:1.95, or at least about 1:1.25:2.

In a further embodiment, the ratio of PD-1 antagonist to TIGIT antagonist to PVRIG antagonist may be at least about 1:1.30:0.25, at least about 1:1.30:0.30, at least about 1:1.30:0.35, at least about 1:1.30:0.40, at least about 1:1.30:0.45, at least about 1:1.30:0.50, at least about 1:1.30:0.55, at least about 1:1.30:0.60, at least about 1:1.30:0.65, at least about 1:1.30:0.70, at least about 1:1.30:0.75, at least about 1:1.30:0.80, at least about 1:1.30:0.85, at least about 1:1.30:0.90, at least about 1:1.30:0.95, at least about 1:1.30:1, at least about 1:1.30:1.05, at least about 1:1.30:1.10, at least about 1:1.30:1.15, at least about 1:1.30:1.20, at least about 1:1.30:1.25, at least about 1:1.30:1.30, at least about 1:1.30:1.35, at least about 1:1.30:1.40, at least about 1:1.30:1.45, at least about 1:1.30:1.50, at least about 1:1.30:1.55, at least about 1:1.30:1.60, at least about 1:1.30:1.65, at least about 1:1.30:1.70, at least about 1:1.30:1.75, at least about 1:1.30:1.80, at least about 1:1.30:1.85, at least about 1:1.30:1.90, at least about 1:1.30:1.95, or at least about 1:1.30:2.

In a further embodiment, the ratio of PD-1 antagonist to TIGIT antagonist to PVRIG antagonist may be at least about 1:1.35:0.25, at least about 1:1.35:0.30, at least about 1:1.35:0.35, at least about 1:1.35:0.40, at least about 1:1.35:0.45, at least about 1:1.35:0.50, at least about 1:1.35:0.55, at least about 1:1.35:0.65, at least about 1:1.35:0.70, at least about 1:1.35:0.75, at least about 1:1.35:0.80, at least about 1:1.35:0.85, at least about 1:1.35:0.90, at least about 1:1.35:0.95, at least about 1:1.35:1, at least about 1:1.35:1.05, at least about 1:1.35:1.10, at least about 1:1.35:1.15, at least about 1:1.35:1.20, at least about 1:1.35:1.25, at least about 1:1.35:1.30, at least about 1:1.35:1.35, at least about 1:1.35:1.40, at least about 1:1.35:1.45, at least about 1:1.35:1.50, at least about 1:1.35:1.55, at least about 1:1.35:1.60, at least about 1:1.35:1.65, at least about 1:1.35:1.70, at least about 1:1.35:1.75, at least about 1:1.35:1.80, at least about 1:1.35:1.85, at least about 1:1.35:1.90, at least about 1:1.35:1.95, or at least about 1:1.35:2.

In a further embodiment, the ratio of PD-1 antagonist to TIGIT antagonist to PVRIG antagonist may be at least about 1:1.40:0.25, at least about 1:1.40:0.30, at least about 1:1.40:0.35, at least about 1:1.40:0.40, at least about 1:1.40:0.45, at least about 1:1.40:0.50, at least about 1:1.40:0.55, at least about 1:1.40:0.60, at least about 1:1.40:0.65, at least about 1:1.40:0.70, at least about 1:1.40:0.75, at least about 1:1.40:0.80, at least about 1:1.40:0.85, at least about 1:1.40:0.90, at least about 1:1.40:0.95, at least about 1:1.40:1, at least about 1:1.40:1.05, at least about 1:1.40:1.10, at least about 1:1.40:1.15, at least about 1:1.40:1.20, at least about 1:1.40:1.25, at least about 1:1.40:1.30, at least about 1:1.40:1.35, at least about 1:1.40:1.40, at least about 1:1.40:1.45, at least about 1:1.40:1.50, at least about 1:1.40:1.55, at least about 1:1.40:1.60, at least about 1:1.40:1.65, at least about 1:1.40:1.70, at least about 1:1.40:1.75, at least about 1:1.40:1.80, at least about 1:1.40:1.85, at least about 1:1.40:1.90, at least about 1:1.40:1.95, or at least about 1:1.40:2.

In a further embodiment, the ratio of PD-1 antagonist to TIGIT antagonist to PVRIG antagonist may be at least about 1:1.45:0.25, at least about 1:1.45:0.30, at least about 1:1.45:0.35, at least about 1:1.45:0.40, at least about 1:1.45:0.45, at least about 1:1.45:0.50, at least about 1:1.45:0.55, at least about 1:1.45:0.60, at least about 1:1.45:0.65, at least about 1:1.45:0.70, at least about 1:1.45:0.75, at least about 1:1.45:0.80, at least about 1:1.45:0.85, at least about 1:1.45:0.90, at least about 1:1.45:0.95, at least about 1:1.45:1, at least about 1:1.45:1.05, at least about 1:1.45:1.10, at least about 1:1.45:1.15, at least about 1:1.45:1.20, at least about 1:1.45:1.25, at least about 1:1.45:1.30, at least about 1:1.45:1.35, at least about 1:1.45:1.40, at least about 1:1.45:1.45, at least about 1:1.45:1.50, at least about 1:1.45:1.55, at least about 1:1.45:1.60, at least about 1:1.45:1.65, at least about 1:1.45:1.70, at least about 1:1.45:1.75, at least about 1:1.45:1.80, at least about 1:1.45:1.85, at least about 1:1.45:1.90, at least about 1:1.45:1.95, or at least about 1:1.45:2.

In a further embodiment, the ratio of PD-1 antagonist to TIGIT antagonist to PVRIG antagonist may be at least about 1:1.50:0.25, at least about 1:1.50:0.30, at least about 1:1.50:0.35, at least about 1:1.50:0.40, at least about 1:1.50:0.45, at least about 1:1.50:0.50, at least about 1:1.50:0.55, at least about 1:1.50:0.60, at least about 1:1.50:0.65, at least about 1:1.50:0.70, at least about 1:1.50:0.75, at least about 1:1.50:0.80, at least about 1:1.50:0.85, at least about 1:1.50:0.90, at least about 1:1.50:0.95, at least about 1:1.50:1, at least about 1:1.50:1.05, at least about 1:1.50:1.10, at least about 1:1.50:1.15, at least about 1:1.50:1.20, at least about 1:1.50:1.25, at least about 1:1.50:1.30, at least about 1:1.50:1.35, at least about 1:1.50:1.40, at least about 1:1.50:1.45, at least about 1:1.50:1.50, at least about 1:1.50:1.55, at least about 1:1.50:1.60, at least about 1:1.50:1.65, at least about 1:1.50:1.70, at least about 1:1.50:1.75, at least about 1:1.50:1.80, at least about 1:1.50:1.85, at least about 1:1.50:1.90, at least about 1:1.50:1.95, or at least about 1:1.50:2.

In a further embodiment, the ratio of PD-1 antagonist to TIGIT antagonist to PVRIG antagonist may be at least about 1:1.55:0.25, at least about 1:1.55:0.30, at least about 1:1.55:0.35, at least about 1:1.55:0.40, at least about 1:1.55:0.45, at least about 1:1.55:0.50, at least about 1:1.55:0.55, at least about 1:1.55:0.60, at least about 1:1.55:0.65, at least about 1:1.55:0.70, at least about 1:1.55:0.75, at least about 1:1.55:0.80, at least about 1:1.55:0.85, at least about 1:1.55:0.90, at least about 1:1.55:0.95, at least about 1:1.55:1, at least about 1:1.55:1.05, at least about 1:1.55:1.10, at least about 1:1.55:1.15, at least about 1:1.55:1.20, at least about 1:1.55:1.25, at least about 1:1.55:1.30, at least about 1:1.55:1.35, at least about 1:1.55:1.40, at least about 1:1.55:1.45, at least about 1:1.55:1.50, at least about 1:1.55:1.55, at least about 1:1.55:1.60, at least about 1:1.55:1.65, at least about 1:1.55:1.70, at least about 1:1.55:1.75, at least about 1:1.55:1.80, at least about 1:1.55:1.85, at least about 1:1.55:1.90, at least about 1:1.55:1.95, or at least about 1:1.55:2.

In a further embodiment, the ratio of PD-1 antagonist to TIGIT antagonist to PVRIG antagonist may be at least about 1:1.60:0.25, at least about 1:1.60:0.30, at least about 1:1.60:0.35, at least about 1:1.60:0.40, at least about 1:1.60:0.45, at least about 1:1.60:0.50, at least about 1:1.60:0.55, at least about 1:1.60:0.60, at least about 1:1.60:0.65, at least about 1:1.60:0.70, at least about 1:1.60:0.75, at least about 1:1.60:0.80, at least about 1:1.60:0.85, at least about 1:1.60:0.90, at least about 1:1.60:0.95, at least about 1:1.60:1, at least about 1:1.60:1.05, at least about 1:1.60:1.10, at least about 1:1.60:1.15, at least about 1:1.60:1.20, at least about 1:1.60:1.25, at least about 1:1.60:1.30, at least about 1:1.60:1.35, at least about 1:1.60:1.40, at least about 1:1.60:1.45, at least about 1:1.60:1.50, at least about 1:1.60:1.55, at least about 1:1.60:1.60, at least about 1:1.60:1.65, at least about 1:1.60:1.70, at least about 1:1.60:1.75, at least about 1:1.60:1.80, at least about 1:1.60:1.85, at least about 1:1.60:1.90, at least about 1:1.60:1.95, or at least about 1:1.60:2.

In a further embodiment, the ratio of PD-1 antagonist to TIGIT antagonist to PVRIG antagonist may be at least about 1:1.65:0.25, at least about 1:1.65:0.30, at least about 1:1.65:0.35, at least about 1:1.65:0.40, at least about 1:1.65:0.45, at least about 1:1.65:0.50, at least about 1:1.65:0.55, at least about 1:1.65:0.60, at least about 1:1.65:0.65, at least about 1:1.65:0.70, at least about 1:1.65:0.75, at least about 1:1.65:0.80, at least about 1:1.65:0.85, at least about 1:1.65:0.90, at least about 1:1.65:0.95, at least about 1:1.65:1, at least about 1:1.65:1.05, at least about 1:1.65:1.10, at least about 1:1.65:1.15, at least about 1:1.65:1.20, at least about 1:1.65:1.25, at least about 1:1.65:1.30, at least about 1:1.65:1.35, at least about 1:1.65:1.40, at least about 1:1.65:1.45, at least about 1:1.65:1.50, at least about 1:1.65:1.55, at least about 1:1.65:1.60, at least about 1:1.65:1.65, at least about 1:1.65:1.70, at least about 1:1.65:1.75, at least about 1:1.65:1.80, at least about 1:1.65:1.85, at least about 1:1.65:1.90, at least about 1:1.65:1.95, or at least about 1:1.65:2.

In a further embodiment, the ratio of PD-1 antagonist to TIGIT antagonist to PVRIG antagonist may be at least about 1:1.70:0.25, at least about 1:1.70:0.30, at least about 1:1.70:0.35, at least about 1:1.70:0.40, at least about 1:1.70:0.45, at least about 1:1.70:0.50, at least about 1:1.70:0.55, at least about 1:1.70:0.60, at least about 1:1.70:0.65, at least about 1:1.70:0.70, at least about 1:1.70:0.75, at least about 1:1.70:0.80, at least about 1:1.70:0.85, at least about 1:1.70:0.90, at least about 1:1.70:0.95, at least about 1:1.70:1, at least about 1:1.70:1.05, at least about 1:1.70:1.10, at least about 1:1.70:1.15, at least about 1:1.70:1.20, at least about 1:1.70:1.25, at least about 1:1.70:1.30, at least about 1:1.70:1.35, at least about 1:1.70:1.40, at least about 1:1.70:1.45, at least about 1:1.70:1.50, at least about 1:1.70:1.55, at least about 1:1.70:1.60, at least about 1:1.70:1.65, at least about 1:1.70:1.70, at least about 1:1.70:1.75, at least about 1:1.70:1.80, at least about 1:1.70:1.85, at least about 1:1.70:1.90, at least about 1:1.70:1.95, or at least about 1:1.70:2.

In a further embodiment, the ratio of PD-1 antagonist to TIGIT antagonist to PVRIG antagonist may be at least about 1:1.75:0.25, at least about 1:1.75:0.30, at least about 1:1.75:0.35, at least about 1:1.75:0.40, at least about 1:1.75:0.45, at least about 1:1.75:0.50, at least about 1:1.75:0.55, at least about 1:1.75:0.60, at least about 1:1.75:0.65, at least about 1:1.75:0.70, at least about 1:1.75:0.75, at least about 1:1.75:0.80, at least about 1:1.75:0.85, at least about 1:1.75:0.90, at least about 1:1.75:0.95, at least about 1:1.75:1, at least about 1:1.75:1.05, at least about 1:1.75:1.10, at least about 1:1.75:1.15, at least about 1:1.75:1.20, at least about 1:1.75:1.25, at least about 1:1.75:1.30, at least about 1:1.75:1.35, at least about 1:1.75:1.40, at least about 1:1.75:1.45, at least about 1:1.75:1.50, at least about 1:1.75:1.55, at least about 1:1.75:1.60, at least about 1:1.75:1.65, at least about 1:1.75:1.70, at least about 1:1.75:1.75, at least about 1:1.75:1.80, at least about 1:1.75:1.85, at least about 1:1.75:1.90, at least about 1:1.75:1.95, or at least about 1:1.75:2.

In a further embodiment, the ratio of PD-1 antagonist to TIGIT antagonist to PVRIG antagonist may be at least about 1:1.80:0.25, at least about 1:1.80:0.30, at least about 1:1.80:0.35, at least about 1:1.80:0.40, at least about 1:1.80:0.45, at least about 1:1.80:0.50, at least about 1:1.80:0.55, at least about 1:1.80:0.60, at least about 1:1.80:0.65, at least about 1:1.80:0.70, at least about 1:1.80:0.75, at least about 1:1.80:0.80, at least about 1:1.80:0.85, at least about 1:1.80:0.90, at least about 1:1.80:0.95, at least about 1:1.80:1, at least about 1:1.80:1.05, at least about 1:1.80:1.10, at least about 1:1.80:1.15, at least about 1:1.80:1.20, at least about 1:1.80:1.25, at least about 1:1.80:1.30, at least about 1:1.80:1.35, at least about 1:1.80:1.40, at least about 1:1.80:1.45, at least about 1:1.80:1.50, at least about 1:1.80:1.55, at least about 1:1.80:1.60, at least about 1:1.80:1.65, at least about 1:1.80:1.70, at least about 1:1.80:1.75, at least about 1:1.80:1.80, at least about 1:1.80:1.85, at least about 1:1.80:1.90, at least about 1:1.80:1.95, or at least about 1:1.80:2.

In a further embodiment, the ratio of PD-1 antagonist to TIGIT antagonist to PVRIG antagonist may be at least about 1:1.85:0.25, at least about 1:1.85:0.30, at least about 1:1.85:0.35, at least about 1:1.85:0.40, at least about 1:1.85:0.45, at least about 1:1.85:0.50, at least about 1:1.85:0.55, at least about 1:1.85:0.60, at least about 1:1.85:0.65, at least about 1:1.85:0.70, at least about 1:1.85:0.75, at least about 1:1.85:0.80, at least about 1:1.85:0.85, at least about 1:1.85:0.90, at least about 1:1.85:0.95, at least about 1:1.85:1, at least about 1:1.85:1.05, at least about 1:1.85:1.10, at least about 1:1.85:1.15, at least about 1:1.85:1.20, at least about 1:1.85:1.25, at least about 1:1.85:1.30, at least about 1:1.85:1.35, at least about 1:1.85:1.40, at least about 1:1.85:1.45, at least about 1:1.85:1.50, at least about 1:1.85:1.55, at least about 1:1.85:1.60, at least about 1:1.85:1.65, at least about 1:1.85:1.70, at least about 1:1.85:1.75, at least about 1:1.85:1.80, at least about 1:1.85:1.85, at least about 1:1.85:1.90, at least about 1:1.85:1.95, or at least about 1:1.85:2.

In a further embodiment, the ratio of PD-1 antagonist to TIGIT antagonist to PVRIG antagonist may be at least about 1:1.90:0.25, at least about 1:1.90:0.30, at least about 1:1.90:0.35, at least about 1:1.90:0.40, at least about 1:1.90:0.45, at least about 1:1.90:0.50, at least about 1:1.90:0.55, at least about 1:1.90:0.60, at least about 1:1.90:0.65, at least about 1:1.90:0.70, at least about 1:1.90:0.75, at least about 1:1.90:0.80, at least about 1:1.90:0.85, at least about 1:1.90:0.90, at least about 1:1.90:0.95, at least about 1:1.90:1, at least about 1:1.90:1.05, at least about 1:1.90:1.10, at least about 1:1.90:1.15, at least about 1:1.90:1.20, at least about 1:1.90:1.25, at least about 1:1.90:1.30, at least about 1:1.90:1.35, at least about 1:1.90:1.40, at least about 1:1.90:1.45, at least about 1:1.90:1.50, at least about 1:1.90:1.55, at least about 1:1.90:1.60, at least about 1:1.90:1.65, at least about 1:1.90:1.70, at least about 1:1.90:1.75, at least about 1:1.90:1.80, at least about 1:1.90:1.85, at least about 1:1.90:1.90, at least about 1:1.90:1.95, or at least about 1:1.90:2.

In a further embodiment, the ratio of PD-1 antagonist to TIGIT antagonist to PVRIG antagonist may be at least about 1:1.95:0.25, at least about 1:1.95:0.30, at least about 1:1.95:0.35, at least about 1:1.95:0.40, at least about 1:1.95:0.45, at least about 1:1.95:0.50, at least about 1:1.95:0.55, at least about 1:1.95:0.60, at least about 1:1.95:0.65, at least about 1:1.95:0.70, at least about 1:1.95:0.75, at least about 1:1.95:0.80, at least about 1:1.95:0.85, at least about 1:1.95:0.90, at least about 1:1.95:0.95, at least about 1:1.95:1, at least about 1:1.95:1.05, at least about 1:1.95:1.10, at least about 1:1.95:1.15, at least about 1:1.95:1.20, at least about 1:1.95:1.25, at least about 1:1.95:1.30, at least about 1:1.95:1.35, at least about 1:1.95:1.40, at least about 1:1.95:1.45, at least about 1:1.95:1.50, at least about 1:1.95:1.55, at least about 1:1.95:1.60, at least about 1:1.95:1.65, at least about 1:1.95:1.70, at least about 1:1.95:1.75, at least about 1:1.95:1.80, at least about 1:1.95:1.85, at least about 1:1.95:1.90, at least about 1:1.95:1.95, or at least about 1:1.95:2.

In a further embodiment, the ratio of PD-1 antagonist to TIGIT antagonist to PVRIG antagonist may be at least about 1:2:0.25, at least about 1:2:0.30, at least about 1:2:0.35, at least about 1:2:0.40, at least about 1:2:0.45, at least about 1:2:0.50, at least about 1:2:0.55, at least about 1:2:0.60, at least about 1:2:0.65, at least about 1:2:0.70, at least about 1:2:0.75, at least about 1:2:0.80, at least about 1:2:0.85, at least about 1:2:0.90, at least about 1:2:0.95, at least about 1:2:1, at least about 1:2:1.05, at least about 1:2:1.10, at least about 1:2:1.15, at least about 1:2:1.20, at least about 1:2:1.25, at least about 1:2:1.30, at least about 1:2:1.35, at least about 1:2:1.40, at least about 1:2:1.45, at least about 1:2:1.50, at least about 1:2:1.55, at least about 1:2:1.60, at least about 1:2:1.65, at least about 1:2:1.70, at least about 1:2:1.75, at least about 1:2:1.80, at least about 1:2:1.85, at least about 1:2:1.90, at least about 1:2:1.95, or at least about 1:2:2.

In a further embodiment, the ratio of PD-1 antagonist to TIGIT antagonist to PVRIG antagonist may be at least about 1:0.25:0.25, at least about 1:0.30:0.25, at least about 1:0.35:0.25, at least about 1:0.40:0.25, at least about 1:0.45:0.25, at least about 1:0.50:0.25, at least about 1:0.55:0.25, at least about 1:0.60:0.25, at least about 1:0.65:0.25, at least about 1:0.70:0.25, at least about 1:0.75:0.25, at least about 1:0.80:0.25, at least about 1:0.85:0.25, at least about 1:0.90:0.25, at least about 1:0.95:0.25, at least about 1:1:0.25, at least about 1:1.05:0.25, at least about 1:1.10:0.25, at least about 1:1.15:0.25, at least about 1:1.20:0.25, at least about 1:1.25:0.25, at least about 1:1.30:0.25, at least about 1:1.35:0.25, at least about 1:1.40:0.25, at least about 1:1.45:0.25, at least about 1:1.50:0.25, at least about 1:1.55:0.25, at least about 1:1.60:0.25, at least about 1:1.65:0.25, at least about 1:1.70:0.25, at least about 1:1.75:0.25, at least about 1:1.80:0.25, at least about 1:1.85:0.25, at least about 1:1.90:0.25, at least about 1:1.95:0.25, or at least about 1:2:0.25.

In a further embodiment, the ratio of PD-1 antagonist to TIGIT antagonist to PVRIG antagonist may be at least about 1:0.25:0.30, at least about 1:0.30:0.30, at least about 1:0.35:0.30, at least about 1:0.40:0.30, at least about 1:0.45:0.30, at least about 1:0.50:0.30, at least about 1:0.55:0.30, at least about 1:0.60:0.30, at least about 1:0.65:0.30, at least about 1:0.70:0.30, at least about 1:0.75:0.30, at least about 1:0.80:0.30, at least about 1:0.85:0.30, at least about 1:0.90:0.30, at least about 1:0.95:0.30, at least about 1:1:0.30, at least about 1:1.05:0.30, at least about 1:1.10:0.30, at least about 1:1.15:0.30, at least about 1:1.20:0.30, at least about 1:1.25:0.30, at least about 1:1.30:0.30, at least about 1:1.35:0.30, at least about 1:1.40:0.30, at least about 1:1.45:0.30, at least about 1:1.50:0.30, at least about 1:1.55:0.30, at least about 1:1.60:0.30, at least about 1:1.65:0.30, at least about 1:1.70:0.30, at least about 1:1.75:0.30, at least about 1:1.80:0.30, at least about 1:1.85:0.30, at least about 1:1.90:0.30, at least about 1:1.95:0.30, or at least about 1:2:0.30.

In a further embodiment, the ratio of PD-1 antagonist to TIGIT antagonist to PVRIG antagonist may be at least about 1:0.25:0.35, at least about 1:0.30:0.35, at least about 1:0.35:0.35, at least about 1:0.40:0.35, at least about 1:0.45:0.35, at least about 1:0.50:0.35, at least about 1:0.55:0.35, at least about 1:0.60:0.35, at least about 1:0.65:0.35, at least about 1:0.70:0.35, at least about 1:0.75:0.35, at least about 1:0.80:0.35, at least about 1:0.85:0.35, at least about 1:0.90:0.35, at least about 1:0.95:0.35, at least about 1:1:0.35, at least about 1:1.05:0.35, at least about 1:1.10:0.35, at least about 1:1.15:0.35, at least about 1:1.20:0.35, at least about 1:1.25:0.35, at least about 1:1.30:0.35, at least about 1:1.35:0.35, at least about 1:1.40:0.35, at least about 1:1.45:0.35, at least about 1:1.50:0.35, at least about 1:1.55:0.35, at least about 1:1.60:0.35, at least about 1:1.65:0.35, at least about 1:1.70:0.35, at least about 1:1.75:0.35, at least about 1:1.80:0.35, at least about 1:1.85:0.35, at least about 1:1.90:0.35, at least about 1:1.95:0.35, or at least about 1:2:0.35.

In a further embodiment, the ratio of PD-1 antagonist to TIGIT antagonist to PVRIG antagonist may be at least about 1:0.25:0.40, at least about 1:0.30:0.40, at least about 1:0.35:0.40, at least about 1:0.40:0.40, at least about 1:0.45:0.40, at least about 1:0.50:0.40, at least about 1:0.55:0.40, at least about 1:0.60:0.40, at least about 1:0.65:0.40, at least about 1:0.70:0.40, at least about 1:0.75:0.40, at least about 1:0.80:0.40, at least about 1:0.85:0.40, at least about 1:0.90:0.40, at least about 1:0.95:0.40, at least about 1:1:0.40, at least about 1:1.05:0.40, at least about 1:1.10:0.40, at least about 1:1.15:0.40, at least about 1:1.20:0.40, at least about 1:1.25:0.40, at least about 1:1.30:0.40, at least about 1:1.35:0.40, at least about 1:1.40:0.40, at least about 1:1.45:0.40, at least about 1:1.50:0.40, at least about 1:1.55:0.40, at least about 1:1.60:0.40, at least about 1:1.65:0.40, at least about 1:1.70:0.40, at least about 1:1.75:0.40, at least about 1:1.80:0.40, at least about 1:1.85:0.40, at least about 1:1.90:0.40, at least about 1:1.95:0.40, or at least about 1:2:0.40.

In a further embodiment, the ratio of PD-1 antagonist to TIGIT antagonist to PVRIG antagonist may be at least about 1:0.25:0.45, at least about 1:0.30:0.45, at least about 1:0.35:0.45, at least about 1:0.40:0.45, at least about 1:0.45:0.45, at least about 1:0.50:0.45, at least about 1:0.55:0.45, at least about 1:0.60:0.45, at least about 1:0.65:0.45, at least about 1:0.70:0.45, at least about 1:0.75:0.45, at least about 1:0.80:0.45, at least about 1:0.85:0.45, at least about 1:0.90:0.45, at least about 1:0.95:0.45, at least about 1:1:0.45, at least about 1:1.05:0.45, at least about 1:1.10:0.45, at least about 1:1.15:0.45, at least about 1:1.20:0.45, at least about 1:1.25:0.45, at least about 1:1.30:0.45, at least about 1:1.35:0.45, at least about 1:1.40:0.45, at least about 1:1.45:0.45, at least about 1:1.50:0.45, at least about 1:1.55:0.45, at least about 1:1.60:0.45, at least about 1:1.65:0.45, at least about 1:1.70:0.45, at least about 1:1.75:0.45, at least about 1:1.80:0.45, at least about 1:1.85:0.45, at least about 1:1.90:0.45, at least about 1:1.95:0.45, or at least about 1:2:0.45.

In a further embodiment, the ratio of PD-1 antagonist to TIGIT antagonist to PVRIG antagonist may be at least about 1:0.25:0.50, at least about 1:0.30:0.50, at least about 1:0.35:0.50, at least about 1:0.40:0.50, at least about 1:0.45:0.50, at least about 1:0.50:0.50, at least about 1:0.55:0.50, at least about 1:0.60:0.50, at least about 1:0.65:0.50, at least about 1:0.70:0.50, at least about 1:0.75:0.50, at least about 1:0.80:0.50, at least about 1:0.85:0.50, at least about 1:0.90:0.50, at least about 1:0.95:0.50, at least about 1:1:0.50, at least about 1:1.05:0.50, at least about 1:1.10:0.50, at least about 1:1.15:0.50, at least about 1:1.20:0.50, at least about 1:1.25:0.50, at least about 1:1.30:0.50, at least about 1:1.35:0.50, at least about 1:1.40:0.50, at least about 1:1.45:0.50, at least about 1:1.50:0.50, at least about 1:1.55:0.50, at least about 1:1.60:0.50, at least about 1:1.65:0.50, at least about 1:1.70:0.50, at least about 1:1.75:0.50, at least about 1:1.80:0.50, at least about 1:1.85:0.50, at least about 1:1.90:0.50, at least about 1:1.95:0.50, or at least about 1:2:0.50.

In a further embodiment, the ratio of PD-1 antagonist to TIGIT antagonist to PVRIG antagonist may be at least about 1:0.25:0.55, at least about 1:0.30:0.55, at least about 1:0.35:0.55, at least about 1:0.40:0.55, at least about 1:0.45:0.55, at least about 1:0.50:0.55, at least about 1:0.55:0.55, at least about 1:0.60:0.55, at least about 1:0.65:0.55, at least about 1:0.70:0.55, at least about 1:0.75:0.55, at least about 1:0.80:0.55, at least about 1:0.85:0.55, at least about 1:0.90:0.55, at least about 1:0.95:0.55, at least about 1:1:0.55, at least about 1:1.05:0.55, at least about 1:1.10:0.55, at least about 1:1.15:0.55, at least about 1:1.20:0.55, at least about 1:1.25:0.55, at least about 1:1.30:0.55, at least about 1:1.35:0.55, at least about 1:1.40:0.55, at least about 1:1.45:0.55, at least about 1:1.50:0.55, at least about 1:1.55:0.55, at least about 1:1.60:0.55, at least about 1:1.65:0.55, at least about 1:1.70:0.55, at least about 1:1.75:0.55, at least about 1:1.80:0.55, at least about 1:1.85:0.55, at least about 1:1.90:0.55, at least about 1:1.95:0.55, or at least about 1:2:0.55.

In a further embodiment, the ratio of PD-1 antagonist to TIGIT antagonist to PVRIG antagonist may be at least about 1:0.25:0.60, at least about 1:0.30:0.60, at least about 1:0.35:0.60, at least about 1:0.40:0.60, at least about 1:0.45:0.60, at least about 1:0.50:0.60, at least about 1:0.55:0.60, at least about 1:0.60:0.60, at least about 1:0.65:0.60, at least about 1:0.70:0.60, at least about 1:0.75:0.60, at least about 1:0.80:0.60, at least about 1:0.85:0.60, at least about 1:0.90:0.60, at least about 1:0.95:0.60, at least about 1:1:0.60, at least about 1:1.05:0.60, at least about 1:1.10:0.60, at least about 1:1.15:0.60, at least about 1:1.20:0.60, at least about 1:1.25:0.60, at least about 1:1.30:0.60, at least about 1:1.35:0.60, at least about 1:1.40:0.60, at least about 1:1.45:0.60, at least about 1:1.50:0.60, at least about 1:1.55:0.60, at least about 1:1.60:0.60, at least about 1:1.65:0.60, at least about 1:1.70:0.60, at least about 1:1.75:0.60, at least about 1:1.80:0.60, at least about 1:1.85:0.60, at least about 1:1.90:0.60, at least about 1:1.95:0.60, or at least about 1:2:0.60.

In a further embodiment, the ratio of PD-1 antagonist to TIGIT antagonist to PVRIG antagonist may be at least about 1:0.25:0.65, at least about 1:0.30:0.65, at least about 1:0.35:0.65, at least about 1:0.40:0.65, at least about 1:0.45:0.65, at least about 1:0.50:0.65, at least about 1:0.55:0.65, at least about 1:0.60:0.65, at least about 1:0.65:0.65, at least about 1:0.70:0.65, at least about 1:0.75:0.65, at least about 1:0.80:0.65, at least about 1:0.85:0.65, at least about 1:0.90:0.65, at least about 1:0.95:0.65, at least about 1:1:0.65, at least about 1:1.05:0.65, at least about 1:1.10:0.65, at least about 1:1.15:0.65, at least about 1:1.20:0.65, at least about 1:1.25:0.65, at least about 1:1.30:0.65, at least about 1:1.35:0.65, at least about 1:1.40:0.65, at least about 1:1.45:0.65, at least about 1:1.50:0.65, at least about 1:1.55:0.65, at least about 1:1.60:0.65, at least about 1:1.65:0.65, at least about 1:1.70:0.65, at least about 1:1.75:0.65, at least about 1:1.80:0.65, at least about 1:1.85:0.65, at least about 1:1.90:0.65, at least about 1:1.95:0.65, or at least about 1:2:0.65.

In a further embodiment, the ratio of PD-1 antagonist to TIGIT antagonist to PVRIG antagonist may be at least about 1:0.25:0.70, at least about 1:0.30:0.70, at least about 1:0.35:0.70, at least about 1:0.40:0.70, at least about 1:0.45:0.70, at least about 1:0.50:0.70, at least about 1:0.55:0.70, at least about 1:0.60:0.70, at least about 1:0.65:0.70, at least about 1:0.70:0.70, at least about 1:0.75:0.70, at least about 1:0.80:0.70, at least about 1:0.85:0.70, at least about 1:0.90:0.70, at least about 1:0.95:0.70, at least about 1:1:0.70, at least about 1:1.05:0.70, at least about 1:1.10:0.70, at least about 1:1.15:0.70, at least about 1:1.20:0.70, at least about 1:1.25:0.70, at least about 1:1.30:0.70, at least about 1:1.35:0.70, at least about 1:1.40:0.70, at least about 1:1.45:0.70, at least about 1:1.50:0.70, at least about 1:1.55:0.70, at least about 1:1.60:0.70, at least about 1:1.65:0.70, at least about 1:1.70:0.70, at least about 1:1.75:0.70, at least about 1:1.80:0.70, at least about 1:1.85:0.70, at least about 1:1.90:0.70, at least about 1:1.95:0.70, or at least about 1:2:0.70.

In a further embodiment, the ratio of PD-1 antagonist to TIGIT antagonist to PVRIG antagonist may be at least about 1:0.25:0.75, at least about 1:0.30:0.75, at least about 1:0.35:0.75, at least about 1:0.40:0.75, at least about 1:0.45:0.75, at least about 1:0.50:0.75, at least about 1:0.55:0.75, at least about 1:0.60:0.75, at least about 1:0.65:0.75, at least about 1:0.70:0.75, at least about 1:0.75:0.75, at least about 1:0.80:0.75, at least about 1:0.85:0.75, at least about 1:0.90:0.75, at least about 1:0.95:0.75, at least about 1:1:0.75, at least about 1:1.05:0.75, at least about 1:1.10:0.75, at least about 1:1.15:0.75, at least about 1:1.20:0.75, at least about 1:1.25:0.75, at least about 1:1.30:0.75, at least about 1:1.35:0.75, at least about 1:1.40:0.75, at least about 1:1.45:0.75, at least about 1:1.50:0.75, at least about 1:1.55:0.75, at least about 1:1.60:0.75, at least about 1:1.65:0.75, at least about 1:1.70:0.75, at least about 1:1.75:0.75, at least about 1:1.80:0.75, at least about 1:1.85:0.75, at least about 1:1.90:0.75, at least about 1:1.95:0.75, or at least about 1:2:0.75.

In a further embodiment, the ratio of PD-1 antagonist to TIGIT antagonist to PVRIG antagonist may be at least about 1:0.25:0.80, at least about 1:0.30:0.80, at least about 1:0.35:0.80, at least about 1:0.40:0.80, at least about 1:0.45:0.80, at least about 1:0.50:0.80, at least about 1:0.55:0.80, at least about 1:0.60:0.80, at least about 1:0.65:0.80, at least about 1:0.70:0.80, at least about 1:0.75:0.80, at least about 1:0.80:0.80, at least about 1:0.85:0.80, at least about 1:0.90:0.80, at least about 1:0.95:0.80, at least about 1:1:0.80, at least about 1:1.05:0.80, at least about 1:1.10:0.80, at least about 1:1.15:0.80, at least about 1:1.20:0.80, at least about 1:1.25:0.80, at least about 1:1.30:0.80, at least about 1:1.35:0.80, at least about 1:1.40:0.80, at least about 1:1.45:0.80, at least about 1:1.50:0.80, at least about 1:1.55:0.80, at least about 1:1.60:0.80, at least about 1:1.65:0.80, at least about 1:1.70:0.80, at least about 1:1.75:0.80, at least about 1:1.80:0.80, at least about 1:1.85:0.80, at least about 1:1.90:0.80, at least about 1:1.95:0.80, or at least about 1:2:0.80.

In a further embodiment, the ratio of PD-1 antagonist to TIGIT antagonist to PVRIG antagonist may be at least about 1:0.25:0.85, at least about 1:0.30:0.85, at least about 1:0.35:0.85, at least about 1:0.40:0.85, at least about 1:0.45:0.85, at least about 1:0.50:0.85, at least about 1:0.55:0.85, at least about 1:0.60:0.85, at least about 1:0.65:0.85, at least about 1:0.70:0.85, at least about 1:0.75:0.85, at least about 1:0.80:0.85, at least about 1:0.85:0.85, at least about 1:0.90:0.85, at least about 1:0.95:0.85, at least about 1:1:0.85, at least about 1:1.05:0.85, at least about 1:1.10:0.85, at least about 1:1.15:0.85, at least about 1:1.20:0.85, at least about 1:1.25:0.85, at least about 1:1.30:0.85, at least about 1:1.35:0.85, at least about 1:1.40:0.85, at least about 1:1.45:0.85, at least about 1:1.50:0.85, at least about 1:1.55:0.85, at least about 1:1.60:0.85, at least about 1:1.65:0.85, at least about 1:1.70:0.85, at least about 1:1.75:0.85, at least about 1:1.80:0.85, at least about 1:1.85:0.85, at least about 1:1.90:0.85, at least about 1:1.95:0.85, or at least about 1:2:0.85.

In a further embodiment, the ratio of PD-1 antagonist to TIGIT antagonist to PVRIG antagonist may be at least about 1:0.25:0.90, at least about 1:0.30:0.90, at least about 1:0.35:0.90, at least about 1:0.40:0.90, at least about 1:0.45:0.90, at least about 1:0.50:0.90, at least about 1:0.55:0.90, at least about 1:0.60:0.90, at least about 1:0.65:0.90, at least about 1:0.70:0.90, at least about 1:0.75:0.90, at least about 1:0.80:0.90, at least about 1:0.85:0.90, at least about 1:0.90:0.90, at least about 1:0.95:0.90, at least about 1:1:0.90, at least about 1:1.05:0.90, at least about 1:1.10:0.90, at least about 1:1.15:0.90, at least about 1:1.20:0.90, at least about 1:1.25:0.90, at least about 1:1.30:0.90, at least about 1:1.35:0.90, at least about 1:1.40:0.90, at least about 1:1.45:0.90, at least about 1:1.50:0.90, at least about 1:1.55:0.90, at least about 1:1.60:0.90, at least about 1:1.65:0.90, at least about 1:1.70:0.90, at least about 1:1.75:0.90, at least about 1:1.80:0.90, at least about 1:1.85:0.90, at least about 1:1.90:0.90, at least about 1:1.95:0.90, or at least about 1:2:0.90.

In a further embodiment, the ratio of PD-1 antagonist to TIGIT antagonist to PVRIG antagonist may be at least about 1:0.25:0.95, at least about 1:0.30:0.95, at least about 1:0.35:0.95, at least about 1:0.40:0.95, at least about 1:0.45:0.95, at least about 1:0.50:0.95, at least about 1:0.55:0.95, at least about 1:0.60:0.95, at least about 1:0.65:0.95, at least about 1:0.70:0.95, at least about 1:0.75:0.95, at least about 1:0.80:0.95, at least about 1:0.85:0.95, at least about 1:0.90:0.95, at least about 1:0.95:0.95, at least about 1:1:0.95, at least about 1:1.05:0.95, at least about 1:1.10:0.95, at least about 1:1.15:0.95, at least about 1:1.20:0.95, at least about 1:1.25:0.95, at least about 1:1.30:0.95, at least about 1:1.35:0.95, at least about 1:1.40:0.95, at least about 1:1.45:0.95, at least about 1:1.50:0.95, at least about 1:1.55:0.95, at least about 1:1.60:0.95, at least about 1:1.65:0.95, at least about 1:1.70:0.95, at least about 1:1.75:0.95, at least about 1:1.80:0.95, at least about 1:1.85:0.95, at least about 1:1.90:0.95, at least about 1:1.95:0.95, or at least about 1:2:0.95.

In a further embodiment, the ratio of PD-1 antagonist to TIGIT antagonist to PVRIG antagonist may be at least about 1:0.25:1, at least about 1:0.30:1, at least about 1:0.35:1, at least about 1:0.40:1, at least about 1:0.45:1, at least about 1:0.50:1, at least about 1:0.55:1, at least about 1:0.60:1, at least about 1:0.65:1, at least about 1:0.70:1, at least about 1:0.75:1, at least about 1:0.80:1, at least about 1:0.85:1, at least about 1:0.90:1, at least about 1:0.95:1, at least about 1:1:1, at least about 1:1.05:1, at least about 1:1.10:1, at least about 1:1.15:1, at least about 1:1.20:1, at least about 1:1.25:1, at least about 1:1.30:1, at least about 1:1.35:1, at least about 1:1.40:1, at least about 1:1.45:1, at least about 1:1.50:1, at least about 1:1.55:1, at least about 1:1.60:1, at least about 1:1.65:1, at least about 1:1.70:1, at least about 1:1.75:1, at least about 1:1.80:1, at least about 1:1.85:1, at least about 1:1.90:1, at least about 1:1.95:1, or at least about 1:2:1.

In a further embodiment, the ratio of PD-1 antagonist to TIGIT antagonist to PVRIG antagonist may be at least about 1:0.25:1.05, at least about 1:0.30:1.05, at least about 1:0.35:1.05, at least about 1:0.40:1.05, at least about 1:0.45:1.05, at least about 1:0.50:1.05, at least about 1:0.55:1.05, at least about 1:0.60:1.05, at least about 1:0.65:1.05, at least about 1:0.70:1.05, at least about 1:0.75:1.05, at least about 1:0.80:1.05, at least about 1:0.85:1.05, at least about 1:0.90:1.05, at least about 1:0.95:1.05, at least about 1:1:1.05, at least about 1:1.05:1.05, at least about 1:1.10:1.05, at least about 1:1.15:1.05, at least about 1:1.20:1.05, at least about 1:1.25:1.05, at least about 1:1.30:1.05, at least about 1:1.35:1.05, at least about 1:1.40:1.05, at least about 1:1.45:1.05, at least about 1:1.50:1.05, at least about 1:1.55:1.05, at least about 1:1.60:1.05, at least about 1:1.65:1.05, at least about 1:1.70:1.05, at least about 1:1.75:1.05, at least about 1:1.80:1.05, at least about 1:1.85:1.05, at least about 1:1.90:1.05, at least about 1:1.95:1.05, or at least about 1:2:1.05.

In a further embodiment, the ratio of PD-1 antagonist to TIGIT antagonist to PVRIG antagonist may be at least about 1:0.25:1.10, at least about 1:0.30:1.10, at least about 1:0.35:1.10, at least about 1:0.40:1.10, at least about 1:0.45:1.10, at least about 1:0.50:1.10, at least about 1:0.55:1.10, at least about 1:0.60:1.10, at least about 1:0.65:1.10, at least about 1:0.70:1.10, at least about 1:0.75:1.10, at least about 1:0.80:1.10, at least about 1:0.85:1.10, at least about 1:0.90:1.10, at least about 1:0.95:1.10, at least about 1:1:1.10, at least about 1:1.05:1.10, at least about 1:1.10:1.10, at least about 1:1.15:1.10, at least about 1:1.20:1.10, at least about 1:1.25:1.10, at least about 1:1.30:1.10, at least about 1:1.35:1.10, at least about 1:1.40:1.10, at least about 1:1.45:1.10, at least about 1:1.50:1.10, at least about 1:1.55:1.10, at least about 1:1.60:1.10, at least about 1:1.65:1.10, at least about 1:1.70:1.10, at least about 1:1.75:1.10, at least about 1:1.80:1.10, at least about 1:1.85:1.10, at least about 1:1.90:1.10, at least about 1:1.95:1.10, or at least about 1:2:1.10.

In a further embodiment, the ratio of PD-1 antagonist to TIGIT antagonist to PVRIG antagonist may be at least about 1:0.25:1.15, at least about 1:0.30:1.15, at least about 1:0.35:1.15, at least about 1:0.40:1.15, at least about 1:0.45:1.15, at least about 1:0.50:1.15, at least about 1:0.55:1.15, at least about 1:0.60:1.15, at least about 1:0.65:1.15, at least about 1:0.70:1.15, at least about 1:0.75:1.15, at least about 1:0.80:1.15, at least about 1:0.85:1.15, at least about 1:0.90:1.15, at least about 1:0.95:1.15, at least about 1:1:1.15, at least about 1:1.05:1.15, at least about 1:1.10:1.15, at least about 1:1.15:1.15, at least about 1:1.20:1.15, at least about 1:1.25:1.15, at least about 1:1.30:1.15, at least about 1:1.35:1.15, at least about 1:1.40:1.15, at least about 1:1.45:1.15, at least about 1:1.50:1.15, at least about 1:1.55:1.15, at least about 1:1.60:1.15, at least about 1:1.65:1.15, at least about 1:1.70:1.15, at least about 1:1.75:1.15, at least about 1:1.80:1.15, at least about 1:1.85:1.15, at least about 1:1.90:1.15, at least about 1:1.95:1.15, or at least about 1:2:1.15.

In a further embodiment, the ratio of PD-1 antagonist to TIGIT antagonist to PVRIG antagonist may be at least about 1:0.25:1.20, at least about 1:0.30:1.20, at least about 1:0.35:1.20, at least about 1:0.40:1.20, at least about 1:0.45:1.20, at least about 1:0.50:1.20, at least about 1:0.55:1.20, at least about 1:0.60:1.20, at least about 1:0.65:1.20, at least about 1:0.70:1.20, at least about 1:0.75:1.20, at least about 1:0.80:1.20, at least about 1:0.85:1.20, at least about 1:0.90:1.20, at least about 1:0.95:1.20, at least about 1:1:1.20, at least about 1:1.05:1.20, at least about 1:1.10:1.20, at least about 1:1.15:1.20, at least about 1:1.20:1.20, at least about 1:1.25:1.20, at least about 1:1.30:1.20, at least about 1:1.35:1.20, at least about 1:1.40:1.20, at least about 1:1.45:1.20, at least about 1:1.50:1.20, at least about 1:1.55:1.20, at least about 1:1.60:1.20, at least about 1:1.65:1.20, at least about 1:1.70:1.20, at least about 1:1.75:1.20, at least about 1:1.80:1.20, at least about 1:1.85:1.20, at least about 1:1.90:1.20, at least about 1:1.95:1.20, or at least about 1:2:1.20.

In a further embodiment, the ratio of PD-1 antagonist to TIGIT antagonist to PVRIG antagonist may be at least about 1:0.25:1.25, at least about 1:0.30:1.25, at least about 1:0.35:1.25, at least about 1:0.40:1.25, at least about 1:0.45:1.25, at least about 1:0.50:1.25, at least about 1:0.55:1.25, at least about 1:0.60:1.25, at least about 1:0.65:1.25, at least about 1:0.70:1.25, at least about 1:0.75:1.25, at least about 1:0.80:1.25, at least about 1:0.85:1.25, at least about 1:0.90:1.25, at least about 1:0.95:1.25, at least about 1:1:1.25, at least about 1:1.05:1.25, at least about 1:1.10:1.25, at least about 1:1.15:1.25, at least about 1:1.20:1.25, at least about 1:1.25:1.25, at least about 1:1.30:1.25, at least about 1:1.35:1.25, at least about 1:1.40:1.25, at least about 1:1.45:1.25, at least about 1:1.50:1.25, at least about 1:1.55:1.25, at least about 1:1.60:1.25, at least about 1:1.65:1.25, at least about 1:1.70:1.25, at least about 1:1.75:1.25, at least about 1:1.80:1.25, at least about 1:1.85:1.25, at least about 1:1.90:1.25, at least about 1:1.95:1.25, or at least about 1:2:1.25.

In a further embodiment, the ratio of PD-1 antagonist to TIGIT antagonist to PVRIG antagonist may be at least about 1:0.25:1.30, at least about 1:0.30:1.30, at least about 1:0.35:1.30, at least about 1:0.40:1.30, at least about 1:0.45:1.30, at least about 1:0.50:1.30, at least about 1:0.55:1.30, at least about 1:0.60:1.30, at least about 1:0.65:1.30, at least about 1:0.70:1.30, at least about 1:0.75:1.30, at least about 1:0.80:1.30, at least about 1:0.85:1.30, at least about 1:0.90:1.30, at least about 1:0.95:1.30, at least about 1:1:1.30, at least about 1:1.05:1.30, at least about 1:1.10:1.30, at least about 1:1.15:1.30, at least about 1:1.20:1.30, at least about 1:1.25:1.30, at least about 1:1.30:1.30, at least about 1:1.35:1.30, at least about 1:1.40:1.30, at least about 1:1.45:1.30, at least about 1:1.50:1.30, at least about 1:1.55:1.30, at least about 1:1.60:1.30, at least about 1:1.65:1.30, at least about 1:1.70:1.30, at least about 1:1.75:1.30, at least about 1:1.80:1.30, at least about 1:1.85:1.30, at least about 1:1.90:1.30, at least about 1:1.95:1.30, or at least about 1:2:1.30.

In a further embodiment, the ratio of PD-1 antagonist to TIGIT antagonist to PVRIG antagonist may be at least about 1:0.25:1.35, at least about 1:0.30:1.35, at least about 1:0.35:1.35, at least about 1:0.40:1.35, at least about 1:0.45:1.35, at least about 1:0.50:1.35, at least about 1:0.55:1.35, at least about 1:0.60:1.35, at least about 1:0.65:1.35, at least about 1:0.70:1.35, at least about 1:0.75:1.35, at least about 1:0.80:1.35, at least about 1:0.85:1.35, at least about 1:0.90:1.35, at least about 1:0.95:1.35, at least about 1:1:1.35, at least about 1:1.05:1.35, at least about 1:1.10:1.35, at least about 1:1.15:1.35, at least about 1:1.20:1.35, at least about 1:1.25:1.35, at least about 1:1.30:1.35, at least about 1:1.35:1.35, at least about 1:1.40:1.35, at least about 1:1.45:1.35, at least about 1:1.50:1.35, at least about 1:1.55:1.35, at least about 1:1.60:1.35, at least about 1:1.65:1.35, at least about 1:1.70:1.35, at least about 1:1.75:1.35, at least about 1:1.80:1.35, at least about 1:1.85:1.35, at least about 1:1.90:1.35, at least about 1:1.95:1.35, or at least about 1:2:1.35.

In a further embodiment, the ratio of PD-1 antagonist to TIGIT antagonist to PVRIG antagonist may be at least about 1:0.25:1.40, at least about 1:0.30:1.40, at least about 1:0.35:1.40, at least about 1:0.40:1.40, at least about 1:0.45:1.40, at least about 1:0.50:1.40, at least about 1:0.55:1.40, at least about 1:0.60:1.40, at least about 1:0.65:1.40, at least about 1:0.70:1.40, at least about 1:0.75:1.40, at least about 1:0.80:1.40, at least about 1:0.85:1.40, at least about 1:0.90:1.40, at least about 1:0.95:1.40, at least about 1:1:1.40, at least about 1:1.05:1.40, at least about 1:1.10:1.40, at least about 1:1.15:1.40, at least about 1:1.20:1.40, at least about 1:1.25:1.40, at least about 1:1.30:1.40, at least about 1:1.35:1.40, at least about 1:1.40:1.40, at least about 1:1.45:1.40, at least about 1:1.50:1.40, at least about 1:1.55:1.40, at least about 1:1.60:1.40, at least about 1:1.65:1.40, at least about 1:1.70:1.40, at least about 1:1.75:1.40, at least about 1:1.80:1.40, at least about 1:1.85:1.40, at least about 1:1.90:1.40, at least about 1:1.95:1.40, or at least about 1:2:1.40.

In a further embodiment, the ratio of PD-1 antagonist to TIGIT antagonist to PVRIG antagonist may be at least about 1:0.25:1.45, at least about 1:0.30:1.45, at least about 1:0.35:1.45, at least about 1:0.40:1.45, at least about 1:0.45:1.45, at least about 1:0.50:1.45, at least about 1:0.55:1.45, at least about 1:0.60:1.45, at least about 1:0.65:1.45, at least about 1:0.70:1.45, at least about 1:0.75:1.45, at least about 1:0.80:1.45, at least about 1:0.85:1.45, at least about 1:0.90:1.45, at least about 1:0.95:1.45, at least about 1:1:1.45, at least about 1:1.05:1.45, at least about 1:1.10:1.45, at least about 1:1.15:1.45, at least about 1:1.20:1.45, at least about 1:1.25:1.45, at least about 1:1.30:1.45, at least about 1:1.35:1.45, at least about 1:1.40:1.45, at least about 1:1.45:1.45, at least about 1:1.50:1.45, at least about 1:1.55:1.45, at least about 1:1.60:1.45, at least about 1:1.65:1.45, at least about 1:1.70:1.45, at least about 1:1.75:1.45, at least about 1:1.80:1.45, at least about 1:1.85:1.45, at least about 1:1.90:1.45, at least about 1:1.95:1.45, or at least about 1:2:1.45.

In a further embodiment, the ratio of PD-1 antagonist to TIGIT antagonist to PVRIG antagonist may be at least about 1:0.25:1.50, at least about 1:0.30:1.50, at least about 1:0.35:1.50, at least about 1:0.40:1.50, at least about 1:0.45:1.50, at least about 1:0.50:1.50, at least about 1:0.55:1.50, at least about 1:0.60:1.50, at least about 1:0.65:1.50, at least about 1:0.70:1.50, at least about 1:0.75:1.50, at least about 1:0.80:1.50, at least about 1:0.85:1.50, at least about 1:0.90:1.50, at least about 1:0.95:1.50, at least about 1:1:1.50, at least about 1:1.05:1.50, at least about 1:1.10:1.50, at least about 1:1.15:1.50, at least about 1:1.20:1.50, at least about 1:1.25:1.50, at least about 1:1.30:1.50, at least about 1:1.35:1.50, at least about 1:1.40:1.50, at least about 1:1.45:1.50, at least about 1:1.50:1.50, at least about 1:1.55:1.50, at least about 1:1.60:1.50, at least about 1:1.65:1.50, at least about 1:1.70:1.50, at least about 1:1.75:1.50, at least about 1:1.80:1.50, at least about 1:1.85:1.50, at least about 1:1.90:1.50, at least about 1:1.95:1.50, or at least about 1:2:1.50.

In a further embodiment, the ratio of PD-1 antagonist to TIGIT antagonist to PVRIG antagonist may be at least about 1:0.25:1.55, at least about 1:0.30:1.55, at least about 1:0.35:1.55, at least about 1:0.40:1.55, at least about 1:0.45:1.55, at least about 1:0.50:1.55, at least about 1:0.55:1.55, at least about 1:0.60:1.55, at least about 1:0.65:1.55, at least about 1:0.70:1.55, at least about 1:0.75:1.55, at least about 1:0.80:1.55, at least about 1:0.85:1.55, at least about 1:0.90:1.55, at least about 1:0.95:1.55, at least about 1:1:1.55, at least about 1:1.05:1.55, at least about 1:1.10:1.55, at least about 1:1.15:1.55, at least about 1:1.20:1.55, at least about 1:1.25:1.55, at least about 1:1.30:1.55, at least about 1:1.35:1.55, at least about 1:1.40:1.55, at least about 1:1.45:1.55, at least about 1:1.50:1.55, at least about 1:1.55:1.55, at least about 1:1.60:1.55, at least about 1:1.65:1.55, at least about 1:1.70:1.55, at least about 1:1.75:1.55, at least about 1:1.80:1.55, at least about 1:1.85:1.55, at least about 1:1.90:1.55, at least about 1:1.95:1.55, or at least about 1:2:1.55.

In a further embodiment, the ratio of PD-1 antagonist to TIGIT antagonist to PVRIG antagonist may be at least about 1:0.25:1.60, at least about 1:0.30:1.60, at least about 1:0.35:1.60, at least about 1:0.40:1.60, at least about 1:0.45:1.60, at least about 1:0.50:1.60, at least about 1:0.55:1.60, at least about 1:0.60:1.60, at least about 1:0.65:1.60, at least about 1:0.70:1.60, at least about 1:0.75:1.60, at least about 1:0.80:1.60, at least about 1:0.85:1.60, at least about 1:0.90:1.60, at least about 1:0.95:1.60, at least about 1:1:1.60, at least about 1:1.05:1.60, at least about 1:1.10:1.60, at least about 1:1.15:1.60, at least about 1:1.20:1.60, at least about 1:1.25:1.60, at least about 1:1.30:1.60, at least about 1:1.35:1.60, at least about 1:1.40:1.60, at least about 1:1.45:1.60, at least about 1:1.50:1.60, at least about 1:1.55:1.60, at least about 1:1.60:1.60, at least about 1:1.65:1.60, at least about 1:1.70:1.60, at least about 1:1.75:1.60, at least about 1:1.80:1.60, at least about 1:1.85:1.60, at least about 1:1.90:1.60, at least about 1:1.95:1.60, or at least about 1:2:1.60.

In a further embodiment, the ratio of PD-1 antagonist to TIGIT antagonist to PVRIG antagonist may be at least about 1:0.25:1.65, at least about 1:0.30:1.65, at least about 1:0.35:1.65, at least about 1:0.40:1.65, at least about 1:0.45:1.65, at least about 1:0.50:1.65, at least about 1:0.55:1.65, at least about 1:0.60:1.65, at least about 1:0.65:1.65, at least about 1:0.70:1.65, at least about 1:0.75:1.65, at least about 1:0.80:1.65, at least about 1:0.85:1.65, at least about 1:0.90:1.65, at least about 1:0.95:1.65, at least about 1:1:1.65, at least about 1:1.05:1.65, at least about 1:1.10:1.65, at least about 1:1.15:1.65, at least about 1:1.20:1.65, at least about 1:1.25:1.65, at least about 1:1.30:1.65, at least about 1:1.35:1.65, at least about 1:1.40:1.65, at least about 1:1.45:1.65, at least about 1:1.50:1.65, at least about 1:1.55:1.65, at least about 1:1.60:1.65, at least about 1:1.65:1.65, at least about 1:1.70:1.65, at least about 1:1.75:1.65, at least about 1:1.80:1.65, at least about 1:1.85:1.65, at least about 1:1.90:1.65, at least about 1:1.95:1.65, or at least about 1:2:1.65.

In a further embodiment, the ratio of PD-1 antagonist to TIGIT antagonist to PVRIG antagonist may be at least about 1:0.25:1.70, at least about 1:0.30:1.70, at least about 1:0.35:1.70, at least about 1:0.40:1.70, at least about 1:0.45:1.70, at least about 1:0.50:1.70, at least about 1:0.55:1.70, at least about 1:0.60:1.70, at least about 1:0.65:1.70, at least about 1:0.70:1.70, at least about 1:0.75:1.70, at least about 1:0.80:1.70, at least about 1:0.85:1.70, at least about 1:0.90:1.70, at least about 1:0.95:1.70, at least about 1:1:1.70, at least about 1:1.05:1.70, at least about 1:1.10:1.70, at least about 1:1.15:1.70, at least about 1:1.20:1.70, at least about 1:1.25:1.70, at least about 1:1.30:1.70, at least about 1:1.35:1.70, at least about 1:1.40:1.70, at least about 1:1.45:1.70, at least about 1:1.50:1.70, at least about 1:1.55:1.70, at least about 1:1.60:1.70, at least about 1:1.65:1.70, at least about 1:1.70:1.70, at least about 1:1.75:1.70, at least about 1:1.80:1.70, at least about 1:1.85:1.70, at least about 1:1.90:1.70, at least about 1:1.95:1.70, or at least about 1:2:1.70.

In a further embodiment, the ratio of PD-1 antagonist to TIGIT antagonist to PVRIG antagonist may be at least about 1:0.25:1.75, at least about 1:0.30:1.75, at least about 1:0.35:1.75, at least about 1:0.40:1.75, at least about 1:0.45:1.75, at least about 1:0.50:1.75, at least about 1:0.55:1.75, at least about 1:0.60:1.75, at least about 1:0.65:1.75, at least about 1:0.70:1.75, at least about 1:0.75:1.75, at least about 1:0.80:1.75, at least about 1:0.85:1.75, at least about 1:0.90:1.75, at least about 1:0.95:1.75, at least about 1:1:1.75, at least about 1:1.05:1.75, at least about 1:1.10:1.75, at least about 1:1.15:1.75, at least about 1:1.20:1.75, at least about 1:1.25:1.75, at least about 1:1.30:1.75, at least about 1:1.35:1.75, at least about 1:1.40:1.75, at least about 1:1.45:1.75, at least about 1:1.50:1.75, at least about 1:1.55:1.75, at least about 1:1.60:1.75, at least about 1:1.65:1.75, at least about 1:1.70:1.75, at least about 1:1.75:1.75, at least about 1:1.80:1.75, at least about 1:1.85:1.75, at least about 1:1.90:1.75, at least about 1:1.95:1.75, or at least about 1:2:1.75.

In a further embodiment, the ratio of PD-1 antagonist to TIGIT antagonist to PVRIG antagonist may be at least about 1:0.25:1.80, at least about 1:0.30:1.80, at least about 1:0.35:1.80, at least about 1:0.40:1.80, at least about 1:0.45:1.80, at least about 1:0.50:1.80, at least about 1:0.55:1.80, at least about 1:0.60:1.80, at least about 1:0.65:1.80, at least about 1:0.70:1.80, at least about 1:0.75:1.80, at least about 1:0.80:1.80, at least about 1:0.85:1.80, at least about 1:0.90:1.80, at least about 1:0.95:1.80, at least about 1:1:1.80, at least about 1:1.05:1.80, at least about 1:1.10:1.80, at least about 1:1.15:1.80, at least about 1:1.20:1.80, at least about 1:1.25:1.80, at least about 1:1.30:1.80, at least about 1:1.35:1.80, at least about 1:1.40:1.80, at least about 1:1.45:1.80, at least about 1:1.50:1.80, at least about 1:1.55:1.80, at least about 1:1.60:1.80, at least about 1:1.65:1.80, at least about 1:1.70:1.80, at least about 1:1.75:1.80, at least about 1:1.80:1.80, at least about 1:1.85:1.80, at least about 1:1.90:1.80, at least about 1:1.95:1.80, or at least about 1:2:1.80.

In a further embodiment, the ratio of PD-1 antagonist to TIGIT antagonist to PVRIG antagonist may be at least about 1:0.25:1.85, at least about 1:0.30:1.85, at least about 1:0.35:1.85, at least about 1:0.40:1.85, at least about 1:0.45:1.85, at least about 1:0.50:1.85, at least about 1:0.55:1.85, at least about 1:0.60:1.85, at least about 1:0.65:1.85, at least about 1:0.70:1.85, at least about 1:0.75:1.85, at least about 1:0.80:1.85, at least about 1:0.85:1.85, at least about 1:0.90:1.85, at least about 1:0.95:1.85, at least about 1:1:1.85, at least about 1:1.05:1.85, at least about 1:1.10:1.85, at least about 1:1.15:1.85, at least about 1:1.20:1.85, at least about 1:1.25:1.85, at least about 1:1.30:1.85, at least about 1:1.35:1.85, at least about 1:1.40:1.85, at least about 1:1.45:1.85, at least about 1:1.50:1.85, at least about 1:1.55:1.85, at least about 1:1.60:1.85, at least about 1:1.65:1.85, at least about 1:1.70:1.85, at least about 1:1.75:1.85, at least about 1:1.80:1.85, at least about 1:1.85:1.85, at least about 1:1.90:1.85, at least about 1:1.95:1.85, or at least about 1:2:1.85.

In a further embodiment, the ratio of PD-1 antagonist to TIGIT antagonist to PVRIG antagonist may be at least about 1:0.25:1.90, at least about 1:0.30:1.90, at least about 1:0.35:1.90, at least about 1:0.40:1.90, at least about 1:0.45:1.90, at least about 1:0.50:1.90, at least about 1:0.55:1.90, at least about 1:0.60:1.90, at least about 1:0.65:1.90, at least about 1:0.70:1.90, at least about 1:0.75:1.90, at least about 1:0.80:1.90, at least about 1:0.85:1.90, at least about 1:0.90:1.90, at least about 1:0.95:1.90, at least about 1:1:1.90, at least about 1:1.05:1.90, at least about 1:1.10:1.90, at least about 1:1.15:1.90, at least about 1:1.20:1.90, at least about 1:1.25:1.90, at least about 1:1.30:1.90, at least about 1:1.35:1.90, at least about 1:1.40:1.90, at least about 1:1.45:1.90, at least about 1:1.50:1.90, at least about 1:1.55:1.90, at least about 1:1.60:1.90, at least about 1:1.65:1.90, at least about 1:1.70:1.90, at least about 1:1.75:1.90, at least about 1:1.80:1.90, at least about 1:1.85:1.90, at least about 1:1.90:1.90, at least about 1:1.95:1.90, or at least about 1:2:1.90.

In a further embodiment, the ratio of PD-1 antagonist to TIGIT antagonist to PVRIG antagonist may be at least about 1:0.25:1.95, at least about 1:0.30:1.95, at least about 1:0.35:1.95, at least about 1:0.40:1.95, at least about 1:0.45:1.95, at least about 1:0.50:1.95, at least about 1:0.55:1.95, at least about 1:0.60:1.95, at least about 1:0.65:1.95, at least about 1:0.70:1.95, at least about 1:0.75:1.95, at least about 1:0.80:1.95, at least about 1:0.85:1.95, at least about 1:0.90:1.95, at least about 1:0.95:1.95, at least about 1:1:1.95, at least about 1:1.05:1.95, at least about 1:1.10:1.95, at least about 1:1.15:1.95, at least about 1:1.20:1.95, at least about 1:1.25:1.95, at least about 1:1.30:1.95, at least about 1:1.35:1.95, at least about 1:1.40:1.95, at least about 1:1.45:1.95, at least about 1:1.50:1.95, at least about 1:1.55:1.95, at least about 1:1.60:1.95, at least about 1:1.65:1.95, at least about 1:1.70:1.95, at least about 1:1.75:1.95, at least about 1:1.80:1.95, at least about 1:1.85:1.95, at least about 1:1.90:1.95, at least about 1:1.95:1.95, or at least about 1:2:1.95.

In a further embodiment, the ratio of PD-1 antagonist to TIGIT antagonist to PVRIG antagonist may be at least about 1:0.25:2, at least about 1:0.30:2, at least about 1:0.35:2, at least about 1:0.40:2, at least about 1:0.45:2, at least about 1:0.50:2, at least about 1:0.55:2, at least about 1:0.60:2, at least about 1:0.65:2, at least about 1:0.70:2, at least about 1:0.75:2, at least about 1:0.80:2, at least about 1:0.85:2, at least about 1:0.90:2, at least about 1:0.95:2, at least about 1:1:2, at least about 1:1.05:2, at least about 1:1.10:2, at least about 1:1.15:2, at least about 1:1.20:2, at least about 1:1.25:2, at least about 1:1.30:2, at least about 1:1.35:2, at least about 1:1.40:2, at least about 1:1.45:2, at least about 1:1.50:2, at least about 1:1.55:2, at least about 1:1.60:2, at least about 1:1.65:2, at least about 1:1.70:2, at least about 1:1.75:2, at least about 1:1.80:2, at least about 1:1.85:2, at least about 1:1.90:2, at least about 1:1.95:2, or at least about 1:2:2.

In a further embodiment, the ratio of PD-1 antagonist to TIGIT antagonist to CD96 antagonist may be at least about 1:0.25:0.25, at least about 1:0.30:0.30, at least about 1:0.35:0.35, at least about 1:0.40:0.40, at least about 1:0.45:0.45, at least about 1:0.50:0.50, at least about 1:0.55:0.55, at least about 1:0.60:0.60, at least about 1:0.65:0.65, at least about 1:0.70:0.70, at least about 1:0.75:0.75, at least about 1:0.80:0.80, at least about 1:0.85:0.85, at least about 1:0.90:0.90, at least about 1:0.95:0.95, at least about 1:1:1, at least about 1:1.05:1.05, at least about 1:1.10:1.10, at least about 1:1.15:1.15, at least about 1:1.20:1.20, at least about 1:1.25:1.25, at least about 1:1.30:1.30, at least about 1:1.35:1.35, at least about 1:1.40:1.40, at least about 1:1.45:1.45, at least about 1:1.50:1.50, at least about 1:1.55:1.55, at least about 1:1.60:1.60, at least about 1:1.65:1.65, at least about 1:1.70:1.70, at least about 1:1.75:1.75, at least about 1:1.80:1.80, at least about 1:1.85:1.85, at least about 1:1.90:1.90, at least about 1:1.95:1.95, or at least about 1:2:2.

In a further embodiment, the ratio of PD-1 antagonist to TIGIT antagonist to CD96 antagonist may be at least about 1:0.25:0.30, at least about 1:0.25:0.35, at least about 1:0.25:0.40, at least about 1:0.25:0.45, at least about 1:0.25:0.50, at least about 1:0.25:0.55, at least about 1:0.25:0.60, at least about 1:0.25:0.65, at least about 1:0.25:0.70, at least about 1:0.25:0.75, at least about 1:0.25:0.80, at least about 1:0.25:0.85, at least about 1:0.25:0.90, at least about 1:0.25:0.95, at least about 1:0.25:1, at least about 1:0.25:1.05, at least about 1:0.25:1.10, at least about 1:0.25:1.15, at least about 1:0.25:1.20, at least about 1:0.25:1.25, at least about 1:0.25:1.30, at least about 1:0.25:1.35, at least about 1:0.25:1.40, at least about 1:0.25:1.45, at least about 1:0.25:1.50, at least about 1:0.25:1.55, at least about 1:0.25:1.60, at least about 1:0.25:1.65, at least about 1:0.25:1.70, at least about 1:0.25:1.75, at least about 1:0.25:1.80, at least about 1:0.25:1.85, at least about 1:0.25:1.90, at least about 1:0.25:1.95, or at least about 1:0.25:2.

In a further embodiment, the ratio of PD-1 antagonist to TIGIT antagonist to CD96 antagonist may be at least about 1:0.30:0.25, at least about 1:0.30:0.30, at least about 1:0.30:0.35, at least about 1:0.30:0.40, at least about 1:0.30:0.45, at least about 1:0.30:0.50, at least about 1:0.30:0.55, at least about 1:0.30:0.60, at least about 1:0.30:0.65, at least about 1:0.30:0.70, at least about 1:0.30:0.75, at least about 1:0.30:0.80, at least about 1:0.30:0.85, at least about 1:0.30:0.90, at least about 1:0.30:0.95, at least about 1:0.30:1, at least about 1:0.30:1.05, at least about 1:0.30:1.10, at least about 1:0.30:1.15, at least about 1:0.30:1.20, at least about 1:0.30:1.25, at least about 1:0.30:1.30, at least about 1:0.30:1.35, at least about 1:0.30:1.40, at least about 1:0.30:1.45, at least about 1:0.30:1.50, at least about 1:0.30:1.55, at least about 1:0.30:1.60, at least about 1:0.30:1.65, at least about 1:0.30:1.70, at least about 1:0.30:1.75, at least about 1:0.30:1.80, at least about 1:0.30:1.85, at least about 1:0.30:1.90, at least about 1:0.30:1.95, or at least about 1:0.30:2.

In a further embodiment, the ratio of PD-1 antagonist to TIGIT antagonist to CD96 antagonist may be at least about 1:0.35:0.25, at least about 1:0.35:0.30, at least about 1:0.35:0.35, at least about 1:0.35:0.40, at least about 1:0.35:0.45, at least about 1:0.35:0.50, at least about 1:0.35:0.55, at least about 1:0.35:0.60, at least about 1:0.35:0.65, at least about 1:0.35:0.70, at least about 1:0.35:0.75, at least about 1:0.35:0.80, at least about 1:0.35:0.85, at least about 1:0.35:0.90, at least about 1:0.35:0.95, at least about 1:0.35:1, at least about 1:0.35:1.05, at least about 1:0.35:1.10, at least about 1:0.35:1.15, at least about 1:0.35:1.20, at least about 1:0.35:1.25, at least about 1:0.35:1.30, at least about 1:0.35:1.35, at least about 1:0.35:1.40, at least about 1:0.35:1.45, at least about 1:0.35:1.50, at least about 1:0.35:1.55, at least about 1:0.35:1.60, at least about 1:0.35:1.65, at least about 1:0.35:1.70, at least about 1:0.35:1.75, at least about 1:0.35:1.80, at least about 1:0.35:1.85, at least about 1:0.35:1.90, at least about 1:0.35:1.95, or at least about 1:0.35:2.

In a further embodiment, the ratio of PD-1 antagonist to TIGIT antagonist to CD96 antagonist may be at least about 1:0.40:0.25, at least about 1:0.40:0.30, at least about 1:0.40:0.35, at least about 1:0.40:0.40, at least about 1:0.40:0.45, at least about 1:0.40:0.50, at least about 1:0.40:0.55, at least about 1:0.40:0.60, at least about 1:0.40:0.65, at least about 1:0.40:0.70, at least about 1:0.40:0.75, at least about 1:0.40:0.80, at least about 1:0.40:0.85, at least about 1:0.40:0.90, at least about 1:0.40:0.95, at least about 1:0.40:1, at least about 1:0.40:1.05, at least about 1:0.40:1.10, at least about 1:0.40:1.15, at least about 1:0.40:1.20, at least about 1:0.40:1.25, at least about 1:0.40:1.30, at least about 1:0.40:1.35, at least about 1:0.40:1.40, at least about 1:0.40:1.45, at least about 1:0.40:1.50, at least about 1:0.40:1.55, at least about 1:0.40:1.60, at least about 1:0.40:1.65, at least about 1:0.40:1.70, at least about 1:0.40:1.75, at least about 1:0.40:1.80, at least about 1:0.40:1.85, at least about 1:0.40:1.90, at least about 1:0.40:1.95, or at least about 1:0.40:2.

In a further embodiment, the ratio of PD-1 antagonist to TIGIT antagonist to CD96 antagonist may be at least about 1:0.45:0.25, at least about 1:0.45:0.30, at least about 1:0.45:0.35, at least about 1:0.45:0.40, at least about 1:0.45:0.45, at least about 1:0.45:0.50, at least about 1:0.45:0.55, at least about 1:0.45:0.60, at least about 1:0.45:0.65, at least about 1:0.45:0.70, at least about 1:0.45:0.75, at least about 1:0.45:0.80, at least about 1:0.45:0.85, at least about 1:0.45:0.90, at least about 1:0.45:0.95, at least about 1:0.45:1, at least about 1:0.45:1.05, at least about 1:0.45:1.10, at least about 1:0.45:1.15, at least about 1:0.45:1.20, at least about 1:0.45:1.25, at least about 1:0.45:1.30, at least about 1:0.45:1.35, at least about 1:0.45:1.40, at least about 1:0.45:1.45, at least about 1:0.45:1.50, at least about 1:0.45:1.55, at least about 1:0.45:1.60, at least about 1:0.45:1.65, at least about 1:0.45:1.70, at least about 1:0.45:1.75, at least about 1:0.45:1.80, at least about 1:0.45:1.85, at least about 1:0.45:1.90, at least about 1:0.45:1.95, or at least about 1:0.45:2.

In a further embodiment, the ratio of PD-1 antagonist to TIGIT antagonist to CD96 antagonist may be at least about 1:0.50:0.25, at least about 1:0.50:0.30, at least about 1:0.50:0.35, at least about 1:0.50:0.40, at least about 1:0.50:0.45, at least about 1:0.50:0.50, at least about 1:0.50:0.55, at least about 1:0.50:0.60, at least about 1:0.50:0.65, at least about 1:0.50:0.70, at least about 1:0.50:0.75, at least about 1:0.50:0.80, at least about 1:0.50:0.85, at least about 1:0.50:0.90, at least about 1:0.50:0.95, at least about 1:0.50:1, at least about 1:0.50:1.05, at least about 1:0.50:1.10, at least about 1:0.50:1.15, at least about 1:0.50:1.20, at least about 1:0.50:1.25, at least about 1:0.50:1.30, at least about 1:0.50:1.35, at least about 1:0.50:1.40, at least about 1:0.50:1.45, at least about 1:0.50:1.50, at least about 1:0.50:1.55, at least about 1:0.50:1.60, at least about 1:0.50:1.65, at least about 1:0.50:1.70, at least about 1:0.50:1.75, at least about 1:0.50:1.80, at least about 1:0.50:1.85, at least about 1:0.50:1.90, at least about 1:0.50:1.95, or at least about 1:0.50:2.

In a further embodiment, the ratio of PD-1 antagonist to TIGIT antagonist to CD96 antagonist may be at least about 1:0.55:0.25, at least about 1:0.55:0.30, at least about 1:0.55:0.35, at least about 1:0.55:0.40, at least about 1:0.55:0.45, at least about 1:0.55:0.50, at least about 1:0.55:0.55, at least about 1:0.55:0.60, at least about 1:0.55:0.65, at least about 1:0.55:0.70, at least about 1:0.55:0.75, at least about 1:0.55:0.80, at least about 1:0.55:0.85, at least about 1:0.55:0.90, at least about 1:0.55:0.95, at least about 1:0.55:1, at least about 1:0.55:1.05, at least about 1:0.55:1.10, at least about 1:0.55:1.15, at least about 1:0.55:1.20, at least about 1:0.55:1.25, at least about 1:0.55:1.30, at least about 1:0.55:1.35, at least about 1:0.55:1.40, at least about 1:0.55:1.45, at least about 1:0.55:1.50, at least about 1:0.55:1.55, at least about 1:0.55:1.60, at least about 1:0.55:1.65, at least about 1:0.55:1.70, at least about 1:0.55:1.75, at least about 1:0.55:1.80, at least about 1:0.55:1.85, at least about 1:0.55:1.90, 1:0.55:1.95, or at least about 1:0.55:2.

In a further embodiment, the ratio of PD-1 antagonist to TIGIT antagonist to CD96 antagonist may be at least about 1:0.60:0.25, at least about 1:0.60:0.30, at least about 1:0.60:0.35, at least about 1:0.60:0.40, at least about 1:0.60:0.45, at least about 1:0.60:0.50, at least about 1:0.60:0.55, at least about 1:0.60:0.60, at least about 1:0.60:0.65, at least about 1:0.60:0.70, at least about 1:0.60:0.75, at least about 1:0.60:0.80, at least about 1:0.60:0.85, at least about 1:0.60:0.90, at least about 1:0.60:0.95, at least about 1:0.60:1, at least about 1:0.60:1.05, at least about 1:0.60:1.10, at least about 1:0.60:1.15, at least about 1:0.60:1.20, at least about 1:0.60:1.25, at least about 1:0.60:1.30, at least about 1:0.60:1.35, at least about 1:0.60:1.40, at least about 1:0.60:1.45, at least about 1:0.60:1.50, at least about 1:0.60:1.55, at least about 1:0.60:1.60, at least about 1:0.60:1.65, at least about 1:0.60:1.70, at least about 1:0.60:1.75, at least about 1:0.60:1.80, at least about 1:0.60:1.85, at least about 1:0.60:1.90, at least about 1:0.60:1.95, or at least about 1:0.60:2.

In a further embodiment, the ratio of PD-1 antagonist to TIGIT antagonist to CD96 antagonist may be at least about 1:0.65:0.25, at least about 1:0.65:0.30, at least about 1:0.65:0.35, at least about 1:0.65:0.40, at least about 1:0.65:0.45, at least about 1:0.65:0.50, at least about 1:0.65:0.55, at least about 1:0.65:0.60, at least about 1:0.65:0.65, at least about 1:0.65:0.70, at least about 1:0.65:0.75, at least about 1:0.65:0.80, at least about 1:0.65:0.85, at least about 1:0.65:0.90, at least about 1:0.65:0.95, at least about 1:0.65:1, at least about 1:0.65:1.05, at least about 1:0.65:1.10, at least about 1:0.65:1.15, at least about 1:0.65:1.20, at least about 1:0.65:1.25, at least about 1:0.65:1.30, at least about 1:0.65:1.35, at least about 1:0.65:1.40, at least about 1:0.65:1.45, at least about 1:0.65:1.50, at least about 1:0.65:1.55, at least about 1:0.65:1.60, at least about 1:0.65:1.65, at least about 1:0.65:1.70, at least about 1:0.65:1.75, at least about 1:0.65:1.80, at least about 1:0.65:1.85, at least about 1:0.65:1.90, at least about 1:0.65:1.95, or at least about 1:0.65:2.

In a further embodiment, the ratio of PD-1 antagonist to TIGIT antagonist to CD96 antagonist may be at least about 1:0.70:0.25, at least about 1:0.70:0.30, at least about 1:0.70:0.35, at least about 1:0.70:0.40, at least about 1:0.70:0.45, at least about 1:0.70:0.50, at least about 1:0.70:0.55, at least about 1:0.70:0.60, at least about 1:0.70:0.65, at least about 1:0.70:0.70, at least about 1:0.70:0.75, at least about 1:0.70:0.80, at least about 1:0.70:0.85, at least about 1:0.70:0.90, at least about 1:0.70:0.95, at least about 1:0.70:1, at least about 1:0.70:1.05, at least about 1:0.70:1.10, at least about 1:0.70:1.15, at least about 1:0.70:1.20, at least about 1:0.70:1.25, at least about 1:0.70:1.30, at least about 1:0.70:1.35, at least about 1:0.70:1.40, at least about 1:0.70:1.45, at least about 1:0.70:1.50, at least about 1:0.70:1.55, at least about 1:0.70:1.60, at least about 1:0.70:1.65, at least about 1:0.70:1.70, at least about 1:0.70:1.75, at least about 1:0.70:1.80, at least about 1:0.70:1.85, at least about 1:0.70:1.90, at least about 1:0.70:1.95, or at least about 1:0.70:2.

In a further embodiment, the ratio of PD-1 antagonist to TIGIT antagonist to CD96 antagonist may be at least about 1:0.75:0.25, at least about 1:0.75:0.30, at least about 1:0.75:0.35, at least about 1:0.75:0.40, at least about 1:0.75:0.45, at least about 1:0.75:0.50, at least about 1:0.75:0.55, at least about 1:0.75:0.60, at least about 1:0.75:0.65, at least about 1:0.75:0.70, at least about 1:0.75:0.75, at least about 1:0.75:0.80, at least about 1:0.75:0.85, at least about 1:0.75:0.90, at least about 1:0.75:0.95, at least about 1:0.75:1, at least about 1:0.75:1.05, at least about 1:0.75:1.10, at least about 1:0.75:1.15, at least about 1:0.75:1.20, at least about 1:0.75:1.25, at least about 1:0.75:1.30, at least about 1:0.75:1.35, at least about 1:0.75:1.40, at least about 1:0.75:1.45, at least about 1:0.75:1.50, at least about 1:0.75:1.55, at least about 1:0.75:1.60, at least about 1:0.75:1.65, at least about 1:0.75:1.70, at least about 1:0.75:1.75, at least about 1:0.75:1.80, at least about 1:0.75:1.85, at least about 1:0.75:1.90, at least about 1:0.75:1.95, or at least about 1:0.75:2.

In a further embodiment, the ratio of PD-1 antagonist to TIGIT antagonist to CD96 antagonist may be at least about 1:0.80:0.25, at least about 1:0.80:0.30, at least about 1:0.80:0.35, at least about 1:0.80:0.40, at least about 1:0.80:0.45, at least about 1:0.80:0.50, at least about 1:0.80:0.55, at least about 1:0.80:0.60, at least about 1:0.80:0.65, at least about 1:0.80:0.70, at least about 1:0.80:0.75, at least about 1:0.80:0.80, at least about 1:0.80:0.85, at least about 1:0.80:0.90, at least about 1:0.80:0.95, at least about 1:0.80:1, at least about 1:0.80:1.05, at least about 1:0.80:1.10, at least about 1:0.80:1.15, at least about 1:0.80:1.20, at least about 1:0.80:1.25, at least about 1:0.80:1.30, at least about 1:0.80:1.35, at least about 1:0.80:1.40, at least about 1:0.80:1.45, at least about 1:0.80:1.50, at least about 1:0.80:1.55, at least about 1:0.80:1.60, at least about 1:0.80:1.65, at least about 1:0.80:1.70, at least about 1:0.80:1.75, at least about 1:0.80:1.80, at least about 1:0.80:1.85, at least about 1:0.80:1.90, at least about 1:0.80:1.95, or at least about 1:0.80:2.

In a further embodiment, the ratio of PD-1 antagonist to TIGIT antagonist to CD96 antagonist may be at least about 1:0.85:0.25, at least about 1:0.85:0.30, at least about 1:0.85:0.35, at least about 1:0.85:0.40, at least about 1:0.85:0.45, at least about 1:0.85:0.50, at least about 1:0.85:0.55, at least about 1:0.85:0.60, at least about 1:0.85:0.65, at least about 1:0.85:0.70, at least about 1:0.85:0.75, at least about 1:0.85:0.80, at least about 1:0.85:0.85, at least about 1:0.85:0.90, at least about 1:0.85:0.95, at least about 1:0.85:1, at least about 1:0.85:1.05, at least about 1:0.85:1.10, at least about 1:0.85:1.15, at least about 1:0.85:1.20, at least about 1:0.85:1.25, at least about 1:0.85:1.30, at least about 1:0.85:1.35, at least about 1:0.85:1.40, at least about 1:0.85:1.45, at least about 1:0.85:1.50, at least about 1:0.85:1.55, at least about 1:0.85:1.60, at least about 1:0.85:1.65, at least about 1:0.85:1.70, at least about 1:0.85:1.75, at least about 1:0.85:1.80, at least about 1:0.85:1.85, at least about 1:0.85:1.90, at least about 1:0.85:1.95, or at least about 1:0.85:2.

In a further embodiment, the ratio of PD-1 antagonist to TIGIT antagonist to CD96 antagonist may be at least about 1:0.90:0.25, at least about 1:0.90:0.30, at least about 1:0.90:0.35, at least about 1:0.90:0.40, at least about 1:0.90:0.45, at least about 1:0.90:0.50, at least about 1:0.90:0.55, at least about 1:0.90:0.60, at least about 1:0.90:0.65, at least about 1:0.90:0.70, at least about 1:0.90:0.75, at least about 1:0.90:0.80, at least about 1:0.90:0.85, at least about 1:0.90:0.90, at least about 1:0.90:0.95, at least about 1:0.90:1, at least about 1:0.90:1.05, at least about 1:0.90:1.10, at least about 1:0.90:1.15, at least about 1:0.90:1.20, at least about 1:0.90:1.25, at least about 1:0.90:1.30, at least about 1:0.90:1.35, at least about 1:0.90:1.40, at least about 1:0.90:1.45, at least about 1:0.90:1.50, at least about 1:0.90:1.55, at least about 1:0.90:1.60, at least about 1:0.90:1.65, at least about 1:0.90:1.70, at least about 1:0.90:1.75, at least about 1:0.90:1.80, at least about 1:0.90:1.85, at least about 1:0.90:1.90, at least about 1:0.90:1.95, or at least about 1:0.90:2.

In a further embodiment, the ratio of PD-1 antagonist to TIGIT antagonist to CD96 antagonist may be at least about 1:0.95:0.25, at least about 1:0.95:0.30, at least about 1:0.95:0.35, at least about 1:0.95:0.40, at least about 1:0.95:0.45, at least about 1:0.95:0.50, at least about 1:0.95:0.55, at least about 1:0.95:0.60, at least about 1:0.95:0.65, at least about 1:0.95:0.70, at least about 1:0.95:0.75, at least about 1:0.95:0.80, at least about 1:0.95:0.85, at least about 1:0.95:0.90, at least about 1:0.95:0.95, at least about 1:0.95:1, at least about 1:0.95:1.05, at least about 1:0.95:1.10, at least about 1:0.95:1.15, at least about 1:0.95:1.20, at least about 1:0.95:1.25, at least about 1:0.95:1.30, at least about 1:0.95:1.35, at least about 1:0.95:1.40, at least about 1:0.95:1.45, at least about 1:0.95:1.50, at least about 1:0.95:1.55, at least about 1:0.95:1.60, at least about 1:0.95:1.65, at least about 1:0.95:1.70, at least about 1:0.95:1.75, at least about 1:0.95:1.80, at least about 1:0.95:1.85, at least about 1:0.95:1.90, at least about 1:0.95:1.95, or at least about 1:0.95:2.

In a further embodiment, the ratio of PD-1 antagonist to TIGIT antagonist to CD96 antagonist may be at least about 1:1:0.25, at least about 1:1:0.30, at least about 1:1:0.35, at least about 1:1:0.40, at least about 1:1:0.45, at least about 1:1:0.50, at least about 1:1:0.55, at least about 1:1:0.60, at least about 1:1:0.65, at least about 1:1:0.70, at least about 1:1:0.75, at least about 1:1:0.80, at least about 1:1:0.85, at least about 1:1:0.90, at least about 1:1:0.95, at least about 1:1:1, at least about 1:1:1.05, at least about 1:1:1.10, at least about 1:1:1.15, at least about 1:1:1.20, at least about 1:1:1.25, at least about 1:1:1.30, at least about 1:1:1.35, at least about 1:1:1.40, at least about 1:1:1.45, at least about 1:1:1.50, at least about 1:1:1.55, at least about 1:1:1.60, at least about 1:1:1.65, at least about 1:1:1.70, at least about 1:1:1.75, at least about 1:1:1.80, at least about 1:1:1.85, at least about 1:1:1.90, at least about 1:1:1.95, or at least about 1:1:2.

In a further embodiment, the ratio of PD-1 antagonist to TIGIT antagonist to CD96 antagonist may be at least about 1:1.05:0.25, at least about 1:1.05:0.30, at least about 1:1.05:0.35, at least about 1:1.05:0.40, at least about 1:1.05:0.45, at least about 1:1.05:0.50, at least about 1:1.05:0.55, at least about 1:1.05:0.60, at least about 1:1.05:0.65, at least about 1:1.05:0.70, at least about 1:1.05:0.75, at least about 1:1.05:0.80, at least about 1:1.05:0.85, at least about 1:1.05:0.90, at least about 1:1.05:0.95, at least about 1:1.05:1, at least about 1:1.05:1.05, at least about 1:1.05:1.10, at least about 1:1.05:1.15, at least about 1:1.05:1.20, at least about 1:1.05:1.25, at least about 1:1.05:1.30, at least about 1:1.05:1.35, at least about 1:1.05:1.40, at least about 1:1.05:1.45, at least about 1:1.05:1.50, at least about 1:1.05:1.55, at least about 1:1.05:1.60, at least about 1:1.05:1.65, at least about 1:1.05:1.70, at least about 1:1.05:1.75, at least about 1:1.05:1.80, at least about 1:1.05:1.85, at least about 1:1.05:1.90, at least about 1:1.05:1.95, or at least about 1:1.05:2.

In a further embodiment, the ratio of PD-1 antagonist to TIGIT antagonist to CD96 antagonist may be at least about 1:1.10:0.25, at least about 1:1.10:0.30, at least about 1:1.10:0.35, at least about 1:1.10:0.40, at least about 1:1.10:0.45, at least about 1:1.10:0.50, at least about 1:1.10:0.55, at least about 1:1.10:0.60, at least about 1:1.10:0.65, at least about 1:1.10:0.70, at least about 1:1.10:0.75, at least about 1:1.10:0.80, at least about 1:1.10:0.85, at least about 1:1.10:0.90, at least about 1:1.10:0.95, at least about 1:1.10:1, at least about 1:1.10:1.05, at least about 1:1.10:1.10, at least about 1:1.10:1.15, at least about 1:1.10:1.20, at least about 1:1.10:1.25, at least about 1:1.10:1.30, at least about 1:1.10:1.35, at least about 1:1.10:1.40, at least about 1:1.10:1.45, at least about 1:1.10:1.50, at least about 1:1.10:1.55, at least about 1:1.10:1.60, at least about 1:1.10:1.65, at least about 1:1.10:1.70, at least about 1:1.10:1.75, at least about 1:1.10:1.80, at least about 1:1.10:1.85, at least about 1:1.10:1.90, at least about 1:1.10:1.95, or at least about 1:1.10:2.

In a further embodiment, the ratio of PD-1 antagonist to TIGIT antagonist to CD96 antagonist may be at least about 1:1.15:0.25, at least about 1:1.15:0.30, at least about 1:1.15:0.35, at least about 1:1.15:0.40, at least about 1:1.15:0.45, at least about 1:1.15:0.50, at least about 1:1.15:0.55, at least about 1:1.15:0.60, at least about 1:1.15:0.65, at least about 1:1.15:0.70, at least about 1:1.15:0.75, at least about 1:1.15:0.80, at least about 1:1.15:0.85, at least about 1:1.15:0.90, at least about 1:1.15:0.95, at least about 1:1.15:1, at least about 1:1.15:1.05, at least about 1:1.15:1.10, at least about 1:1.15:1.15, at least about 1:1.15:1.20, at least about 1:1.15:1.25, at least about 1:1.15:1.30, at least about 1:1.15:1.35, at least about 1:1.15:1.40, at least about 1:1.15:1.45, at least about 1:1.15:1.50, at least about 1:1.15:1.55, at least about 1:1.15:1.60, at least about 1:1.15:1.65, at least about 1:1.15:1.70, at least about 1:1.15:1.75, at least about 1:1.15:1.80, at least about 1:1.15:1.85, at least about 1:1.15:1.90, at least about 1:1.15:1.95, or at least about 1:1.15:2.

In a further embodiment, the ratio of PD-1 antagonist to TIGIT antagonist to CD96 antagonist may be at least about 1:1.20:0.25, at least about 1:1.20:0.30, at least about 1:1.20:0.35, at least about 1:1.20:0.40, at least about 1:1.20:0.45, at least about 1:1.20:0.50, at least about 1:1.20:0.55, at least about 1:1.20:0.60, at least about 1:1.20:0.65, at least about 1:1.20:0.70, at least about 1:1.20:0.75, at least about 1:1.20:0.80, at least about 1:1.20:0.85, at least about 1:1.20:0.90, at least about 1:1.20:0.95, at least about 1:1.20:1, at least about 1:1.20:1.05, at least about 1:1.20:1.10, at least about 1:1.20:1.15, at least about 1:1.20:1.20, at least about 1:1.20:1.25, at least about 1:1.20:1.30, at least about 1:1.20:1.35, at least about 1:1.20:1.40, at least about 1:1.20:1.45, at least about 1:1.20:1.50, at least about 1:1.20:1.55, at least about 1:1.20:1.60, at least about 1:1.20:1.65, at least about 1:1.20:1.70, at least about 1:1.20:1.75, at least about 1:1.20:1.80, at least about 1:1.20:1.85, at least about 1:1.20:1.90, at least about 1:1.20:1.95, or at least about 1:1.20:2.

In a further embodiment, the ratio of PD-1 antagonist to TIGIT antagonist to CD96 antagonist may be at least about 1:1.25:0.25, at least about 1:1.25:0.30, at least about 1:1.25:0.35, at least about 1:1.25:0.40, at least about 1:1.25:0.45, at least about 1:1.25:0.50, at least about 1:1.25:0.55, at least about 1:1.25:0.60, at least about 1:1.25:0.65, at least about 1:1.25:0.70, at least about 1:1.25:0.75, at least about 1:1.25:0.80, at least about 1:1.25:0.85, at least about 1:1.25:0.90, at least about 1:1.25:0.95, at least about 1:1.25:1, at least about 1:1.25:1.05, at least about 1:1.25:1.10, at least about 1:1.25:1.15, at least about 1:1.25:1.20, at least about 1:1.25:1.25, at least about 1:1.25:1.30, at least about 1:1.25:1.35, at least about 1:1.25:1.40, at least about 1:1.25:1.45, at least about 1:1.25:1.50, at least about 1:1.25:1.55, at least about 1:1.25:1.60, at least about 1:1.25:1.65, at least about 1:1.25:1.70, at least about 1:1.25:1.75, at least about 1:1.25:1.80, at least about 1:1.25:1.85, at least about 1:1.25:1.90, at least about 1:1.25:1.95, or at least about 1:1.25:2.

In a further embodiment, the ratio of PD-1 antagonist to TIGIT antagonist to CD96 antagonist may be at least about 1:1.30:0.25, at least about 1:1.30:0.30, at least about 1:1.30:0.35, at least about 1:1.30:0.40, at least about 1:1.30:0.45, at least about 1:1.30:0.50, at least about 1:1.30:0.55, at least about 1:1.30:0.60, at least about 1:1.30:0.65, at least about 1:1.30:0.70, at least about 1:1.30:0.75, at least about 1:1.30:0.80, at least about 1:1.30:0.85, at least about 1:1.30:0.90, at least about 1:1.30:0.95, at least about 1:1.30:1, at least about 1:1.30:1.05, at least about 1:1.30:1.10, at least about 1:1.30:1.15, at least about 1:1.30:1.20, at least about 1:1.30:1.25, at least about 1:1.30:1.30, at least about 1:1.30:1.35, at least about 1:1.30:1.40, at least about 1:1.30:1.45, at least about 1:1.30:1.50, at least about 1:1.30:1.55, at least about 1:1.30:1.60, at least about 1:1.30:1.65, at least about 1:1.30:1.70, at least about 1:1.30:1.75, at least about 1:1.30:1.80, at least about 1:1.30:1.85, at least about 1:1.30:1.90, at least about 1:1.30:1.95, or at least about 1:1.30:2.

In a further embodiment, the ratio of PD-1 antagonist to TIGIT antagonist to CD96 antagonist may be at least about 1:1.35:0.25, at least about 1:1.35:0.30, at least about 1:1.35:0.35, at least about 1:1.35:0.40, at least about 1:1.35:0.45, at least about 1:1.35:0.50, at least about 1:1.35:0.55, at least about 1:1.35:0.65, at least about 1:1.35:0.70, at least about 1:1.35:0.75, at least about 1:1.35:0.80, at least about 1:1.35:0.85, at least about 1:1.35:0.90, at least about 1:1.35:0.95, at least about 1:1.35:1, at least about 1:1.35:1.05, at least about 1:1.35:1.10, at least about 1:1.35:1.15, at least about 1:1.35:1.20, at least about 1:1.35:1.25, at least about 1:1.35:1.30, at least about 1:1.35:1.35, at least about 1:1.35:1.40, at least about 1:1.35:1.45, at least about 1:1.35:1.50, at least about 1:1.35:1.55, at least about 1:1.35:1.60, at least about 1:1.35:1.65, at least about 1:1.35:1.70, at least about 1:1.35:1.75, at least about 1:1.35:1.80, at least about 1:1.35:1.85, at least about 1:1.35:1.90, at least about 1:1.35:1.95, or at least about 1:1.35:2.

In a further embodiment, the ratio of PD-1 antagonist to TIGIT antagonist to CD96 antagonist may be at least about 1:1.40:0.25, at least about 1:1.40:0.30, at least about 1:1.40:0.35, at least about 1:1.40:0.40, at least about 1:1.40:0.45, at least about 1:1.40:0.50, at least about 1:1.40:0.55, at least about 1:1.40:0.60, at least about 1:1.40:0.65, at least about 1:1.40:0.70, at least about 1:1.40:0.75, at least about 1:1.40:0.80, at least about 1:1.40:0.85, at least about 1:1.40:0.90, at least about 1:1.40:0.95, at least about 1:1.40:1, at least about 1:1.40:1.05, at least about 1:1.40:1.10, at least about 1:1.40:1.15, at least about 1:1.40:1.20, at least about 1:1.40:1.25, at least about 1:1.40:1.30, at least about 1:1.40:1.35, at least about 1:1.40:1.40, at least about 1:1.40:1.45, at least about 1:1.40:1.50, at least about 1:1.40:1.55, at least about 1:1.40:1.60, at least about 1:1.40:1.65, at least about 1:1.40:1.70, at least about 1:1.40:1.75, at least about 1:1.40:1.80, at least about 1:1.40:1.85, at least about 1:1.40:1.90, at least about 1:1.40:1.95, or at least about 1:1.40:2.

In a further embodiment, the ratio of PD-1 antagonist to TIGIT antagonist to CD96 antagonist may be at least about 1:1.45:0.25, at least about 1:1.45:0.30, at least about 1:1.45:0.35, at least about 1:1.45:0.40, at least about 1:1.45:0.45, at least about 1:1.45:0.50, at least about 1:1.45:0.55, at least about 1:1.45:0.60, at least about 1:1.45:0.65, at least about 1:1.45:0.70, at least about 1:1.45:0.75, at least about 1:1.45:0.80, at least about 1:1.45:0.85, at least about 1:1.45:0.90, at least about 1:1.45:0.95, at least about 1:1.45:1, at least about 1:1.45:1.05, at least about 1:1.45:1.10, at least about 1:1.45:1.15, at least about 1:1.45:1.20, at least about 1:1.45:1.25, at least about 1:1.45:1.30, at least about 1:1.45:1.35, at least about 1:1.45:1.40, at least about 1:1.45:1.45, at least about 1:1.45:1.50, at least about 1:1.45:1.55, at least about 1:1.45:1.60, at least about 1:1.45:1.65, at least about 1:1.45:1.70, at least about 1:1.45:1.75, at least about 1:1.45:1.80, at least about 1:1.45:1.85, at least about 1:1.45:1.90, at least about 1:1.45:1.95, or at least about 1:1.45:2.

In a further embodiment, the ratio of PD-1 antagonist to TIGIT antagonist to CD96 antagonist may be at least about 1:1.50:0.25, at least about 1:1.50:0.30, at least about 1:1.50:0.35, at least about 1:1.50:0.40, at least about 1:1.50:0.45, at least about 1:1.50:0.50, at least about 1:1.50:0.55, at least about 1:1.50:0.60, at least about 1:1.50:0.65, at least about 1:1.50:0.70, at least about 1:1.50:0.75, at least about 1:1.50:0.80, at least about 1:1.50:0.85, at least about 1:1.50:0.90, at least about 1:1.50:0.95, at least about 1:1.50:1, at least about 1:1.50:1.05, at least about 1:1.50:1.10, at least about 1:1.50:1.15, at least about 1:1.50:1.20, at least about 1:1.50:1.25, at least about 1:1.50:1.30, at least about 1:1.50:1.35, at least about 1:1.50:1.40, at least about 1:1.50:1.45, at least about 1:1.50:1.50, at least about 1:1.50:1.55, at least about 1:1.50:1.60, at least about 1:1.50:1.65, at least about 1:1.50:1.70, at least about 1:1.50:1.75, at least about 1:1.50:1.80, at least about 1:1.50:1.85, at least about 1:1.50:1.90, at least about 1:1.50:1.95, or at least about 1:1.50:2.

In a further embodiment, the ratio of PD-1 antagonist to TIGIT antagonist to CD96 antagonist may be at least about 1:1.55:0.25, at least about 1:1.55:0.30, at least about 1:1.55:0.35, at least about 1:1.55:0.40, at least about 1:1.55:0.45, at least about 1:1.55:0.50, at least about 1:1.55:0.55, at least about 1:1.55:0.60, at least about 1:1.55:0.65, at least about 1:1.55:0.70, at least about 1:1.55:0.75, at least about 1:1.55:0.80, at least about 1:1.55:0.85, at least about 1:1.55:0.90, at least about 1:1.55:0.95, at least about 1:1.55:1, at least about 1:1.55:1.05, at least about 1:1.55:1.10, at least about 1:1.55:1.15, at least about 1:1.55:1.20, at least about 1:1.55:1.25, at least about 1:1.55:1.30, at least about 1:1.55:1.35, at least about 1:1.55:1.40, at least about 1:1.55:1.45, at least about 1:1.55:1.50, at least about 1:1.55:1.55, at least about 1:1.55:1.60, at least about 1:1.55:1.65, at least about 1:1.55:1.70, at least about 1:1.55:1.75, at least about 1:1.55:1.80, at least about 1:1.55:1.85, at least about 1:1.55:1.90, at least about 1:1.55:1.95, or at least about 1:1.55:2.

In a further embodiment, the ratio of PD-1 antagonist to TIGIT antagonist to CD96 antagonist may be at least about 1:1.60:0.25, at least about 1:1.60:0.30, at least about 1:1.60:0.35, at least about 1:1.60:0.40, at least about 1:1.60:0.45, at least about 1:1.60:0.50, at least about 1:1.60:0.55, at least about 1:1.60:0.60, at least about 1:1.60:0.65, at least about 1:1.60:0.70, at least about 1:1.60:0.75, at least about 1:1.60:0.80, at least about 1:1.60:0.85, at least about 1:1.60:0.90, at least about 1:1.60:0.95, at least about 1:1.60:1, at least about 1:1.60:1.05, at least about 1:1.60:1.10, at least about 1:1.60:1.15, at least about 1:1.60:1.20, at least about 1:1.60:1.25, at least about 1:1.60:1.30, at least about 1:1.60:1.35, at least about 1:1.60:1.40, at least about 1:1.60:1.45, at least about 1:1.60:1.50, at least about 1:1.60:1.55, at least about 1:1.60:1.60, at least about 1:1.60:1.65, at least about 1:1.60:1.70, at least about 1:1.60:1.75, at least about 1:1.60:1.80, at least about 1:1.60:1.85, at least about 1:1.60:1.90, at least about 1:1.60:1.95, or at least about 1:1.60:2.

In a further embodiment, the ratio of PD-1 antagonist to TIGIT antagonist to CD96 antagonist may be at least about 1:1.65:0.25, at least about 1:1.65:0.30, at least about 1:1.65:0.35, at least about 1:1.65:0.40, at least about 1:1.65:0.45, at least about 1:1.65:0.50, at least about 1:1.65:0.55, at least about 1:1.65:0.60, at least about 1:1.65:0.65, at least about 1:1.65:0.70, at least about 1:1.65:0.75, at least about 1:1.65:0.80, at least about 1:1.65:0.85, at least about 1:1.65:0.90, at least about 1:1.65:0.95, at least about 1:1.65:1, at least about 1:1.65:1.05, at least about 1:1.65:1.10, at least about 1:1.65:1.15, at least about 1:1.65:1.20, at least about 1:1.65:1.25, at least about 1:1.65:1.30, at least about 1:1.65:1.35, at least about 1:1.65:1.40, at least about 1:1.65:1.45, at least about 1:1.65:1.50, at least about 1:1.65:1.55, at least about 1:1.65:1.60, at least about 1:1.65:1.65, at least about 1:1.65:1.70, at least about 1:1.65:1.75, at least about 1:1.65:1.80, at least about 1:1.65:1.85, at least about 1:1.65:1.90, at least about 1:1.65:1.95, or at least about 1:1.65:2.

In a further embodiment, the ratio of PD-1 antagonist to TIGIT antagonist to CD96 antagonist may be at least about 1:1.70:0.25, at least about 1:1.70:0.30, at least about 1:1.70:0.35, at least about 1:1.70:0.40, at least about 1:1.70:0.45, at least about 1:1.70:0.50, at least about 1:1.70:0.55, at least about 1:1.70:0.60, at least about 1:1.70:0.65, at least about 1:1.70:0.70, at least about 1:1.70:0.75, at least about 1:1.70:0.80, at least about 1:1.70:0.85, at least about 1:1.70:0.90, at least about 1:1.70:0.95, at least about 1:1.70:1, at least about 1:1.70:1.05, at least about 1:1.70:1.10, at least about 1:1.70:1.15, at least about 1:1.70:1.20, at least about 1:1.70:1.25, at least about 1:1.70:1.30, at least about 1:1.70:1.35, at least about 1:1.70:1.40, at least about 1:1.70:1.45, at least about 1:1.70:1.50, at least about 1:1.70:1.55, at least about 1:1.70:1.60, at least about 1:1.70:1.65, at least about 1:1.70:1.70, at least about 1:1.70:1.75, at least about 1:1.70:1.80, at least about 1:1.70:1.85, at least about 1:1.70:1.90, at least about 1:1.70:1.95, or at least about 1:1.70:2.

In a further embodiment, the ratio of PD-1 antagonist to TIGIT antagonist to CD96 antagonist may be at least about 1:1.75:0.25, at least about 1:1.75:0.30, at least about 1:1.75:0.35, at least about 1:1.75:0.40, at least about 1:1.75:0.45, at least about 1:1.75:0.50, at least about 1:1.75:0.55, at least about 1:1.75:0.60, at least about 1:1.75:0.65, at least about 1:1.75:0.70, at least about 1:1.75:0.75, at least about 1:1.75:0.80, at least about 1:1.75:0.85, at least about 1:1.75:0.90, at least about 1:1.75:0.95, at least about 1:1.75:1, at least about 1:1.75:1.05, at least about 1:1.75:1.10, at least about 1:1.75:1.15, at least about 1:1.75:1.20, at least about 1:1.75:1.25, at least about 1:1.75:1.30, at least about 1:1.75:1.35, at least about 1:1.75:1.40, at least about 1:1.75:1.45, at least about 1:1.75:1.50, at least about 1:1.75:1.55, at least about 1:1.75:1.60, at least about 1:1.75:1.65, at least about 1:1.75:1.70, at least about 1:1.75:1.75, at least about 1:1.75:1.80, at least about 1:1.75:1.85, at least about 1:1.75:1.90, at least about 1:1.75:1.95, or at least about 1:1.75:2.

In a further embodiment, the ratio of PD-1 antagonist to TIGIT antagonist to CD96 antagonist may be at least about 1:1.80:0.25, at least about 1:1.80:0.30, at least about 1:1.80:0.35, at least about 1:1.80:0.40, at least about 1:1.80:0.45, at least about 1:1.80:0.50, at least about 1:1.80:0.55, at least about 1:1.80:0.60, at least about 1:1.80:0.65, at least about 1:1.80:0.70, at least about 1:1.80:0.75, at least about 1:1.80:0.80, at least about 1:1.80:0.85, at least about 1:1.80:0.90, at least about 1:1.80:0.95, at least about 1:1.80:1, at least about 1:1.80:1.05, at least about 1:1.80:1.10, at least about 1:1.80:1.15, at least about 1:1.80:1.20, at least about 1:1.80:1.25, at least about 1:1.80:1.30, at least about 1:1.80:1.35, at least about 1:1.80:1.40, at least about 1:1.80:1.45, at least about 1:1.80:1.50, at least about 1:1.80:1.55, at least about 1:1.80:1.60, at least about 1:1.80:1.65, at least about 1:1.80:1.70, at least about 1:1.80:1.75, at least about 1:1.80:1.80, at least about 1:1.80:1.85, at least about 1:1.80:1.90, at least about 1:1.80:1.95, or at least about 1:1.80:2.

In a further embodiment, the ratio of PD-1 antagonist to TIGIT antagonist to CD96 antagonist may be at least about 1:1.85:0.25, at least about 1:1.85:0.30, at least about 1:1.85:0.35, at least about 1:1.85:0.40, at least about 1:1.85:0.45, at least about 1:1.85:0.50, at least about 1:1.85:0.55, at least about 1:1.85:0.60, at least about 1:1.85:0.65, at least about 1:1.85:0.70, at least about 1:1.85:0.75, at least about 1:1.85:0.80, at least about 1:1.85:0.85, at least about 1:1.85:0.90, at least about 1:1.85:0.95, at least about 1:1.85:1, at least about 1:1.85:1.05, at least about 1:1.85:1.10, at least about 1:1.85:1.15, at least about 1:1.85:1.20, at least about 1:1.85:1.25, at least about 1:1.85:1.30, at least about 1:1.85:1.35, at least about 1:1.85:1.40, at least about 1:1.85:1.45, at least about 1:1.85:1.50, at least about 1:1.85:1.55, at least about 1:1.85:1.60, at least about 1:1.85:1.65, at least about 1:1.85:1.70, at least about 1:1.85:1.75, at least about 1:1.85:1.80, at least about 1:1.85:1.85, at least about 1:1.85:1.90, at least about 1:1.85:1.95, or at least about 1:1.85:2.

In a further embodiment, the ratio of PD-1 antagonist to TIGIT antagonist to CD96 antagonist may be at least about 1:1.90:0.25, at least about 1:1.90:0.30, at least about 1:1.90:0.35, at least about 1:1.90:0.40, at least about 1:1.90:0.45, at least about 1:1.90:0.50, at least about 1:1.90:0.55, at least about 1:1.90:0.60, at least about 1:1.90:0.65, at least about 1:1.90:0.70, at least about 1:1.90:0.75, at least about 1:1.90:0.80, at least about 1:1.90:0.85, at least about 1:1.90:0.90, at least about 1:1.90:0.95, at least about 1:1.90:1, at least about 1:1.90:1.05, at least about 1:1.90:1.10, at least about 1:1.90:1.15, at least about 1:1.90:1.20, at least about 1:1.90:1.25, at least about 1:1.90:1.30, at least about 1:1.90:1.35, at least about 1:1.90:1.40, at least about 1:1.90:1.45, at least about 1:1.90:1.50, at least about 1:1.90:1.55, at least about 1:1.90:1.60, at least about 1:1.90:1.65, at least about 1:1.90:1.70, at least about 1:1.90:1.75, at least about 1:1.90:1.80, at least about 1:1.90:1.85, at least about 1:1.90:1.90, at least about 1:1.90:1.95, or at least about 1:1.90:2.

In a further embodiment, the ratio of PD-1 antagonist to TIGIT antagonist to CD96 antagonist may be at least about 1:1.95:0.25, at least about 1:1.95:0.30, at least about 1:1.95:0.35, at least about 1:1.95:0.40, at least about 1:1.95:0.45, at least about 1:1.95:0.50, at least about 1:1.95:0.55, at least about 1:1.95:0.60, at least about 1:1.95:0.65, at least about 1:1.95:0.70, at least about 1:1.95:0.75, at least about 1:1.95:0.80, at least about 1:1.95:0.85, at least about 1:1.95:0.90, at least about 1:1.95:0.95, at least about 1:1.95:1, at least about 1:1.95:1.05, at least about 1:1.95:1.10, at least about 1:1.95:1.15, at least about 1:1.95:1.20, at least about 1:1.95:1.25, at least about 1:1.95:1.30, at least about 1:1.95:1.35, at least about 1:1.95:1.40, at least about 1:1.95:1.45, at least about 1:1.95:1.50, at least about 1:1.95:1.55, at least about 1:1.95:1.60, at least about 1:1.95:1.65, at least about 1:1.95:1.70, at least about 1:1.95:1.75, at least about 1:1.95:1.80, at least about 1:1.95:1.85, at least about 1:1.95:1.90, at least about 1:1.95:1.95, or at least about 1:1.95:2.

In a further embodiment, the ratio of PD-1 antagonist to TIGIT antagonist to CD96 antagonist may be at least about 1:2:0.25, at least about 1:2:0.30, at least about 1:2:0.35, at least about 1:2:0.40, at least about 1:2:0.45, at least about 1:2:0.50, at least about 1:2:0.55, at least about 1:2:0.60, at least about 1:2:0.65, at least about 1:2:0.70, at least about 1:2:0.75, at least about 1:2:0.80, at least about 1:2:0.85, at least about 1:2:0.90, at least about 1:2:0.95, at least about 1:2:1, at least about 1:2:1.05, at least about 1:2:1.10, at least about 1:2:1.15, at least about 1:2:1.20, at least about 1:2:1.25, at least about 1:2:1.30, at least about 1:2:1.35, at least about 1:2:1.40, at least about 1:2:1.45, at least about 1:2:1.50, at least about 1:2:1.55, at least about 1:2:1.60, at least about 1:2:1.65, at least about 1:2:1.70, at least about 1:2:1.75, at least about 1:2:1.80, at least about 1:2:1.85, at least about 1:2:1.90, at least about 1:2:1.95, or at least about 1:2:2.

In a further embodiment, the ratio of PD-1 antagonist to TIGIT antagonist to CD96 antagonist may be at least about 1:0.25:0.25, at least about 1:0.30:0.25, at least about 1:0.35:0.25, at least about 1:0.40:0.25, at least about 1:0.45:0.25, at least about 1:0.50:0.25, at least about 1:0.55:0.25, at least about 1:0.60:0.25, at least about 1:0.65:0.25, at least about 1:0.70:0.25, at least about 1:0.75:0.25, at least about 1:0.80:0.25, at least about 1:0.85:0.25, at least about 1:0.90:0.25, at least about 1:0.95:0.25, at least about 1:1:0.25, at least about 1:1.05:0.25, at least about 1:1.10:0.25, at least about 1:1.15:0.25, at least about 1:1.20:0.25, at least about 1:1.25:0.25, at least about 1:1.30:0.25, at least about 1:1.35:0.25, at least about 1:1.40:0.25, at least about 1:1.45:0.25, at least about 1:1.50:0.25, at least about 1:1.55:0.25, at least about 1:1.60:0.25, at least about 1:1.65:0.25, at least about 1:1.70:0.25, at least about 1:1.75:0.25, at least about 1:1.80:0.25, at least about 1:1.85:0.25, at least about 1:1.90:0.25, at least about 1:1.95:0.25, or at least about 1:2:0.25.

In a further embodiment, the ratio of PD-1 antagonist to TIGIT antagonist to CD96 antagonist may be at least about 1:0.25:0.30, at least about 1:0.30:0.30, at least about 1:0.35:0.30, at least about 1:0.40:0.30, at least about 1:0.45:0.30, at least about 1:0.50:0.30, at least about 1:0.55:0.30, at least about 1:0.60:0.30, at least about 1:0.65:0.30, at least about 1:0.70:0.30, at least about 1:0.75:0.30, at least about 1:0.80:0.30, at least about 1:0.85:0.30, at least about 1:0.90:0.30, at least about 1:0.95:0.30, at least about 1:1:0.30, at least about 1:1.05:0.30, at least about 1:1.10:0.30, at least about 1:1.15:0.30, at least about 1:1.20:0.30, at least about 1:1.25:0.30, at least about 1:1.30:0.30, at least about 1:1.35:0.30, at least about 1:1.40:0.30, at least about 1:1.45:0.30, at least about 1:1.50:0.30, at least about 1:1.55:0.30, at least about 1:1.60:0.30, at least about 1:1.65:0.30, at least about 1:1.70:0.30, at least about 1:1.75:0.30, at least about 1:1.80:0.30, at least about 1:1.85:0.30, at least about 1:1.90:0.30, at least about 1:1.95:0.30, or at least about 1:2:0.30.

In a further embodiment, the ratio of PD-1 antagonist to TIGIT antagonist to CD96 antagonist may be at least about 1:0.25:0.35, at least about 1:0.30:0.35, at least about 1:0.35:0.35, at least about 1:0.40:0.35, at least about 1:0.45:0.35, at least about 1:0.50:0.35, at least about 1:0.55:0.35, at least about 1:0.60:0.35, at least about 1:0.65:0.35, at least about 1:0.70:0.35, at least about 1:0.75:0.35, at least about 1:0.80:0.35, at least about 1:0.85:0.35, at least about 1:0.90:0.35, at least about 1:0.95:0.35, at least about 1:1:0.35, at least about 1:1.05:0.35, at least about 1:1.10:0.35, at least about 1:1.15:0.35, at least about 1:1.20:0.35, at least about 1:1.25:0.35, at least about 1:1.30:0.35, at least about 1:1.35:0.35, at least about 1:1.40:0.35, at least about 1:1.45:0.35, at least about 1:1.50:0.35, at least about 1:1.55:0.35, at least about 1:1.60:0.35, at least about 1:1.65:0.35, at least about 1:1.70:0.35, at least about 1:1.75:0.35, at least about 1:1.80:0.35, at least about 1:1.85:0.35, at least about 1:1.90:0.35, at least about 1:1.95:0.35, or at least about 1:2:0.35.

In a further embodiment, the ratio of PD-1 antagonist to TIGIT antagonist to CD96 antagonist may be at least about 1:0.25:0.40, at least about 1:0.30:0.40, at least about 1:0.35:0.40, at least about 1:0.40:0.40, at least about 1:0.45:0.40, at least about 1:0.50:0.40, at least about 1:0.55:0.40, at least about 1:0.60:0.40, at least about 1:0.65:0.40, at least about 1:0.70:0.40, at least about 1:0.75:0.40, at least about 1:0.80:0.40, at least about 1:0.85:0.40, at least about 1:0.90:0.40, at least about 1:0.95:0.40, at least about 1:1:0.40, at least about 1:1.05:0.40, at least about 1:1.10:0.40, at least about 1:1.15:0.40, at least about 1:1.20:0.40, at least about 1:1.25:0.40, at least about 1:1.30:0.40, at least about 1:1.35:0.40, at least about 1:1.40:0.40, at least about 1:1.45:0.40, at least about 1:1.50:0.40, at least about 1:1.55:0.40, at least about 1:1.60:0.40, at least about 1:1.65:0.40, at least about 1:1.70:0.40, at least about 1:1.75:0.40, at least about 1:1.80:0.40, at least about 1:1.85:0.40, at least about 1:1.90:0.40, at least about 1:1.95:0.40, or at least about 1:2:0.40.

In a further embodiment, the ratio of PD-1 antagonist to TIGIT antagonist to CD96 antagonist may be at least about 1:0.25:0.45, at least about 1:0.30:0.45, at least about 1:0.35:0.45, at least about 1:0.40:0.45, at least about 1:0.45:0.45, at least about 1:0.50:0.45, at least about 1:0.55:0.45, at least about 1:0.60:0.45, at least about 1:0.65:0.45, at least about 1:0.70:0.45, at least about 1:0.75:0.45, at least about 1:0.80:0.45, at least about 1:0.85:0.45, at least about 1:0.90:0.45, at least about 1:0.95:0.45, at least about 1:1:0.45, at least about 1:1.05:0.45, at least about 1:1.10:0.45, at least about 1:1.15:0.45, at least about 1:1.20:0.45, at least about 1:1.25:0.45, at least about 1:1.30:0.45, at least about 1:1.35:0.45, at least about 1:1.40:0.45, at least about 1:1.45:0.45, at least about 1:1.50:0.45, at least about 1:1.55:0.45, at least about 1:1.60:0.45, at least about 1:1.65:0.45, at least about 1:1.70:0.45, at least about 1:1.75:0.45, at least about 1:1.80:0.45, at least about 1:1.85:0.45, at least about 1:1.90:0.45, at least about 1:1.95:0.45, or at least about 1:2:0.45.

In a further embodiment, the ratio of PD-1 antagonist to TIGIT antagonist to CD96 antagonist may be at least about 1:0.25:0.50, at least about 1:0.30:0.50, at least about 1:0.35:0.50, at least about 1:0.40:0.50, at least about 1:0.45:0.50, at least about 1:0.50:0.50, at least about 1:0.55:0.50, at least about 1:0.60:0.50, at least about 1:0.65:0.50, at least about 1:0.70:0.50, at least about 1:0.75:0.50, at least about 1:0.80:0.50, at least about 1:0.85:0.50, at least about 1:0.90:0.50, at least about 1:0.95:0.50, at least about 1:1:0.50, at least about 1:1.05:0.50, at least about 1:1.10:0.50, at least about 1:1.15:0.50, at least about 1:1.20:0.50, at least about 1:1.25:0.50, at least about 1:1.30:0.50, at least about 1:1.35:0.50, at least about 1:1.40:0.50, at least about 1:1.45:0.50, at least about 1:1.50:0.50, at least about 1:1.55:0.50, at least about 1:1.60:0.50, at least about 1:1.65:0.50, at least about 1:1.70:0.50, at least about 1:1.75:0.50, at least about 1:1.80:0.50, at least about 1:1.85:0.50, at least about 1:1.90:0.50, at least about 1:1.95:0.50, or at least about 1:2:0.50.

In a further embodiment, the ratio of PD-1 antagonist to TIGIT antagonist to CD96 antagonist may be at least about 1:0.25:0.55, at least about 1:0.30:0.55, at least about 1:0.35:0.55, at least about 1:0.40:0.55, at least about 1:0.45:0.55, at least about 1:0.50:0.55, at least about 1:0.55:0.55, at least about 1:0.60:0.55, at least about 1:0.65:0.55, at least about 1:0.70:0.55, at least about 1:0.75:0.55, at least about 1:0.80:0.55, at least about 1:0.85:0.55, at least about 1:0.90:0.55, at least about 1:0.95:0.55, at least about 1:1:0.55, at least about 1:1.05:0.55, at least about 1:1.10:0.55, at least about 1:1.15:0.55, at least about 1:1.20:0.55, at least about 1:1.25:0.55, at least about 1:1.30:0.55, at least about 1:1.35:0.55, at least about 1:1.40:0.55, at least about 1:1.45:0.55, at least about 1:1.50:0.55, at least about 1:1.55:0.55, at least about 1:1.60:0.55, at least about 1:1.65:0.55, at least about 1:1.70:0.55, at least about 1:1.75:0.55, at least about 1:1.80:0.55, at least about 1:1.85:0.55, at least about 1:1.90:0.55, at least about 1:1.95:0.55, or at least about 1:2:0.55.

In a further embodiment, the ratio of PD-1 antagonist to TIGIT antagonist to CD96 antagonist may be at least about 1:0.25:0.60, at least about 1:0.30:0.60, at least about 1:0.35:0.60, at least about 1:0.40:0.60, at least about 1:0.45:0.60, at least about 1:0.50:0.60, at least about 1:0.55:0.60, at least about 1:0.60:0.60, at least about 1:0.65:0.60, at least about 1:0.70:0.60, at least about 1:0.75:0.60, at least about 1:0.80:0.60, at least about 1:0.85:0.60, at least about 1:0.90:0.60, at least about 1:0.95:0.60, at least about 1:1:0.60, at least about 1:1.05:0.60, at least about 1:1.10:0.60, at least about 1:1.15:0.60, at least about 1:1.20:0.60, at least about 1:1.25:0.60, at least about 1:1.30:0.60, at least about 1:1.35:0.60, at least about 1:1.40:0.60, at least about 1:1.45:0.60, at least about 1:1.50:0.60, at least about 1:1.55:0.60, at least about 1:1.60:0.60, at least about 1:1.65:0.60, at least about 1:1.70:0.60, at least about 1:1.75:0.60, at least about 1:1.80:0.60, at least about 1:1.85:0.60, at least about 1:1.90:0.60, at least about 1:1.95:0.60, or at least about 1:2:0.60.

In a further embodiment, the ratio of PD-1 antagonist to TIGIT antagonist to CD96 antagonist may be at least about 1:0.25:0.65, at least about 1:0.30:0.65, at least about 1:0.35:0.65, at least about 1:0.40:0.65, at least about 1:0.45:0.65, at least about 1:0.50:0.65, at least about 1:0.55:0.65, at least about 1:0.60:0.65, at least about 1:0.65:0.65, at least about 1:0.70:0.65, at least about 1:0.75:0.65, at least about 1:0.80:0.65, at least about 1:0.85:0.65, at least about 1:0.90:0.65, at least about 1:0.95:0.65, at least about 1:1:0.65, at least about 1:1.05:0.65, at least about 1:1.10:0.65, at least about 1:1.15:0.65, at least about 1:1.20:0.65, at least about 1:1.25:0.65, at least about 1:1.30:0.65, at least about 1:1.35:0.65, at least about 1:1.40:0.65, at least about 1:1.45:0.65, at least about 1:1.50:0.65, at least about 1:1.55:0.65, at least about 1:1.60:0.65, at least about 1:1.65:0.65, at least about 1:1.70:0.65, at least about 1:1.75:0.65, at least about 1:1.80:0.65, at least about 1:1.85:0.65, at least about 1:1.90:0.65, at least about 1:1.95:0.65, or at least about 1:2:0.65.

In a further embodiment, the ratio of PD-1 antagonist to TIGIT antagonist to CD96 antagonist may be at least about 1:0.25:0.70, at least about 1:0.30:0.70, at least about 1:0.35:0.70, at least about 1:0.40:0.70, at least about 1:0.45:0.70, at least about 1:0.50:0.70, at least about 1:0.55:0.70, at least about 1:0.60:0.70, at least about 1:0.65:0.70, at least about 1:0.70:0.70, at least about 1:0.75:0.70, at least about 1:0.80:0.70, at least about 1:0.85:0.70, at least about 1:0.90:0.70, at least about 1:0.95:0.70, at least about 1:1:0.70, at least about 1:1.05:0.70, at least about 1:1.10:0.70, at least about 1:1.15:0.70, at least about 1:1.20:0.70, at least about 1:1.25:0.70, at least about 1:1.30:0.70, at least about 1:1.35:0.70, at least about 1:1.40:0.70, at least about 1:1.45:0.70, at least about 1:1.50:0.70, at least about 1:1.55:0.70, at least about 1:1.60:0.70, at least about 1:1.65:0.70, at least about 1:1.70:0.70, at least about 1:1.75:0.70, at least about 1:1.80:0.70, at least about 1:1.85:0.70, at least about 1:1.90:0.70, at least about 1:1.95:0.70, or at least about 1:2:0.70.

In a further embodiment, the ratio of PD-1 antagonist to TIGIT antagonist to CD96 antagonist may be at least about 1:0.25:0.75, at least about 1:0.30:0.75, at least about 1:0.35:0.75, at least about 1:0.40:0.75, at least about 1:0.45:0.75, at least about 1:0.50:0.75, at least about 1:0.55:0.75, at least about 1:0.60:0.75, at least about 1:0.65:0.75, at least about 1:0.70:0.75, at least about 1:0.75:0.75, at least about 1:0.80:0.75, at least about 1:0.85:0.75, at least about 1:0.90:0.75, at least about 1:0.95:0.75, at least about 1:1:0.75, at least about 1:1.05:0.75, at least about 1:1.10:0.75, at least about 1:1.15:0.75, at least about 1:1.20:0.75, at least about 1:1.25:0.75, at least about 1:1.30:0.75, at least about 1:1.35:0.75, at least about 1:1.40:0.75, at least about 1:1.45:0.75, at least about 1:1.50:0.75, at least about 1:1.55:0.75, at least about 1:1.60:0.75, at least about 1:1.65:0.75, at least about 1:1.70:0.75, at least about 1:1.75:0.75, at least about 1:1.80:0.75, at least about 1:1.85:0.75, at least about 1:1.90:0.75, at least about 1:1.95:0.75, or at least about 1:2:0.75.

In a further embodiment, the ratio of PD-1 antagonist to TIGIT antagonist to CD96 antagonist may be at least about 1:0.25:0.80, at least about 1:0.30:0.80, at least about 1:0.35:0.80, at least about 1:0.40:0.80, at least about 1:0.45:0.80, at least about 1:0.50:0.80, at least about 1:0.55:0.80, at least about 1:0.60:0.80, at least about 1:0.65:0.80, at least about 1:0.70:0.80, at least about 1:0.75:0.80, at least about 1:0.80:0.80, at least about 1:0.85:0.80, at least about 1:0.90:0.80, at least about 1:0.95:0.80, at least about 1:1:0.80, at least about 1:1.05:0.80, at least about 1:1.10:0.80, at least about 1:1.15:0.80, at least about 1:1.20:0.80, at least about 1:1.25:0.80, at least about 1:1.30:0.80, at least about 1:1.35:0.80, at least about 1:1.40:0.80, at least about 1:1.45:0.80, at least about 1:1.50:0.80, at least about 1:1.55:0.80, at least about 1:1.60:0.80, at least about 1:1.65:0.80, at least about 1:1.70:0.80, at least about 1:1.75:0.80, at least about 1:1.80:0.80, at least about 1:1.85:0.80, at least about 1:1.90:0.80, at least about 1:1.95:0.80, or at least about 1:2:0.80.

In a further embodiment, the ratio of PD-1 antagonist to TIGIT antagonist to CD96 antagonist may be at least about 1:0.25:0.85, at least about 1:0.30:0.85, at least about 1:0.35:0.85, at least about 1:0.40:0.85, at least about 1:0.45:0.85, at least about 1:0.50:0.85, at least about 1:0.55:0.85, at least about 1:0.60:0.85, at least about 1:0.65:0.85, at least about 1:0.70:0.85, at least about 1:0.75:0.85, at least about 1:0.80:0.85, at least about 1:0.85:0.85, at least about 1:0.90:0.85, at least about 1:0.95:0.85, at least about 1:1:0.85, at least about 1:1.05:0.85, at least about 1:1.10:0.85, at least about 1:1.15:0.85, at least about 1:1.20:0.85, at least about 1:1.25:0.85, at least about 1:1.30:0.85, at least about 1:1.35:0.85, at least about 1:1.40:0.85, at least about 1:1.45:0.85, at least about 1:1.50:0.85, at least about 1:1.55:0.85, at least about 1:1.60:0.85, at least about 1:1.65:0.85, at least about 1:1.70:0.85, at least about 1:1.75:0.85, at least about 1:1.80:0.85, at least about 1:1.85:0.85, at least about 1:1.90:0.85, at least about 1:1.95:0.85, or at least about 1:2:0.85.

In a further embodiment, the ratio of PD-1 antagonist to TIGIT antagonist to CD96 antagonist may be at least about 1:0.25:0.90, at least about 1:0.30:0.90, at least about 1:0.35:0.90, at least about 1:0.40:0.90, at least about 1:0.45:0.90, at least about 1:0.50:0.90, at least about 1:0.55:0.90, at least about 1:0.60:0.90, at least about 1:0.65:0.90, at least about 1:0.70:0.90, at least about 1:0.75:0.90, at least about 1:0.80:0.90, at least about 1:0.85:0.90, at least about 1:0.90:0.90, at least about 1:0.95:0.90, at least about 1:1:0.90, at least about 1:1.05:0.90, at least about 1:1.10:0.90, at least about 1:1.15:0.90, at least about 1:1.20:0.90, at least about 1:1.25:0.90, at least about 1:1.30:0.90, at least about 1:1.35:0.90, at least about 1:1.40:0.90, at least about 1:1.45:0.90, at least about 1:1.50:0.90, at least about 1:1.55:0.90, at least about 1:1.60:0.90, at least about 1:1.65:0.90, at least about 1:1.70:0.90, at least about 1:1.75:0.90, at least about 1:1.80:0.90, at least about 1:1.85:0.90, at least about 1:1.90:0.90, at least about 1:1.95:0.90, or at least about 1:2:0.90.

In a further embodiment, the ratio of PD-1 antagonist to TIGIT antagonist to CD96 antagonist may be at least about 1:0.25:0.95, at least about 1:0.30:0.95, at least about 1:0.35:0.95, at least about 1:0.40:0.95, at least about 1:0.45:0.95, at least about 1:0.50:0.95, at least about 1:0.55:0.95, at least about 1:0.60:0.95, at least about 1:0.65:0.95, at least about 1:0.70:0.95, at least about 1:0.75:0.95, at least about 1:0.80:0.95, at least about 1:0.85:0.95, at least about 1:0.90:0.95, at least about 1:0.95:0.95, at least about 1:1:0.95, at least about 1:1.05:0.95, at least about 1:1.10:0.95, at least about 1:1.15:0.95, at least about 1:1.20:0.95, at least about 1:1.25:0.95, at least about 1:1.30:0.95, at least about 1:1.35:0.95, at least about 1:1.40:0.95, at least about 1:1.45:0.95, at least about 1:1.50:0.95, at least about 1:1.55:0.95, at least about 1:1.60:0.95, at least about 1:1.65:0.95, at least about 1:1.70:0.95, at least about 1:1.75:0.95, at least about 1:1.80:0.95, at least about 1:1.85:0.95, at least about 1:1.90:0.95, at least about 1:1.95:0.95, or at least about 1:2:0.95.

In a further embodiment, the ratio of PD-1 antagonist to TIGIT antagonist to CD96 antagonist may be at least about 1:0.25:1, at least about 1:0.30:1, at least about 1:0.35:1, at least about 1:0.40:1, at least about 1:0.45:1, at least about 1:0.50:1, at least about 1:0.55:1, at least about 1:0.60:1, at least about 1:0.65:1, at least about 1:0.70:1, at least about 1:0.75:1, at least about 1:0.80:1, at least about 1:0.85:1, at least about 1:0.90:1, at least about 1:0.95:1, at least about 1:1:1, at least about 1:1.05:1, at least about 1:1.10:1, at least about 1:1.15:1, at least about 1:1.20:1, at least about 1:1.25:1, at least about 1:1.30:1, at least about 1:1.35:1, at least about 1:1.40:1, at least about 1:1.45:1, at least about 1:1.50:1, at least about 1:1.55:1, at least about 1:1.60:1, at least about 1:1.65:1, at least about 1:1.70:1, at least about 1:1.75:1, at least about 1:1.80:1, at least about 1:1.85:1, at least about 1:1.90:1, at least about 1:1.95:1, or at least about 1:2:1.

In a further embodiment, the ratio of PD-1 antagonist to TIGIT antagonist to CD96 antagonist may be at least about 1:0.25:1.05, at least about 1:0.30:1.05, at least about 1:0.35:1.05, at least about 1:0.40:1.05, at least about 1:0.45:1.05, at least about 1:0.50:1.05, at least about 1:0.55:1.05, at least about 1:0.60:1.05, at least about 1:0.65:1.05, at least about 1:0.70:1.05, at least about 1:0.75:1.05, at least about 1:0.80:1.05, at least about 1:0.85:1.05, at least about 1:0.90:1.05, at least about 1:0.95:1.05, at least about 1:1:1.05, at least about 1:1.05:1.05, at least about 1:1.10:1.05, at least about 1:1.15:1.05, at least about 1:1.20:1.05, at least about 1:1.25:1.05, at least about 1:1.30:1.05, at least about 1:1.35:1.05, at least about 1:1.40:1.05, at least about 1:1.45:1.05, at least about 1:1.50:1.05, at least about 1:1.55:1.05, at least about 1:1.60:1.05, at least about 1:1.65:1.05, at least about 1:1.70:1.05, at least about 1:1.75:1.05, at least about 1:1.80:1.05, at least about 1:1.85:1.05, at least about 1:1.90:1.05, at least about 1:1.95:1.05, or at least about 1:2:1.05.

In a further embodiment, the ratio of PD-1 antagonist to TIGIT antagonist to CD96 antagonist may be at least about 1:0.25:1.10, at least about 1:0.30:1.10, at least about 1:0.35:1.10, at least about 1:0.40:1.10, at least about 1:0.45:1.10, at least about 1:0.50:1.10, at least about 1:0.55:1.10, at least about 1:0.60:1.10, at least about 1:0.65:1.10, at least about 1:0.70:1.10, at least about 1:0.75:1.10, at least about 1:0.80:1.10, at least about 1:0.85:1.10, at least about 1:0.90:1.10, at least about 1:0.95:1.10, at least about 1:1:1.10, at least about 1:1.05:1.10, at least about 1:1.10:1.10, at least about 1:1.15:1.10, at least about 1:1.20:1.10, at least about 1:1.25:1.10, at least about 1:1.30:1.10, at least about 1:1.35:1.10, at least about 1:1.40:1.10, at least about 1:1.45:1.10, at least about 1:1.50:1.10, at least about 1:1.55:1.10, at least about 1:1.60:1.10, at least about 1:1.65:1.10, at least about 1:1.70:1.10, at least about 1:1.75:1.10, at least about 1:1.80:1.10, at least about 1:1.85:1.10, at least about 1:1.90:1.10, at least about 1:1.95:1.10, or at least about 1:2:1.10.

In a further embodiment, the ratio of PD-1 antagonist to TIGIT antagonist to CD96 antagonist may be at least about 1:0.25:1.15, at least about 1:0.30:1.15, at least about 1:0.35:1.15, at least about 1:0.40:1.15, at least about 1:0.45:1.15, at least about 1:0.50:1.15, at least about 1:0.55:1.15, at least about 1:0.60:1.15, at least about 1:0.65:1.15, at least about 1:0.70:1.15, at least about 1:0.75:1.15, at least about 1:0.80:1.15, at least about 1:0.85:1.15, at least about 1:0.90:1.15, at least about 1:0.95:1.15, at least about 1:1:1.15, at least about 1:1.05:1.15, at least about 1:1.10:1.15, at least about 1:1.15:1.15, at least about 1:1.20:1.15, at least about 1:1.25:1.15, at least about 1:1.30:1.15, at least about 1:1.35:1.15, at least about 1:1.40:1.15, at least about 1:1.45:1.15, at least about 1:1.50:1.15, at least about 1:1.55:1.15, at least about 1:1.60:1.15, at least about 1:1.65:1.15, at least about 1:1.70:1.15, at least about 1:1.75:1.15, at least about 1:1.80:1.15, at least about 1:1.85:1.15, at least about 1:1.90:1.15, at least about 1:1.95:1.15, or at least about 1:2:1.15.

In a further embodiment, the ratio of PD-1 antagonist to TIGIT antagonist to CD96 antagonist may be at least about 1:0.25:1.20, at least about 1:0.30:1.20, at least about 1:0.35:1.20, at least about 1:0.40:1.20, at least about 1:0.45:1.20, at least about 1:0.50:1.20, at least about 1:0.55:1.20, at least about 1:0.60:1.20, at least about 1:0.65:1.20, at least about 1:0.70:1.20, at least about 1:0.75:1.20, at least about 1:0.80:1.20, at least about 1:0.85:1.20, at least about 1:0.90:1.20, at least about 1:0.95:1.20, at least about 1:1:1.20, at least about 1:1.05:1.20, at least about 1:1.10:1.20, at least about 1:1.15:1.20, at least about 1:1.20:1.20, at least about 1:1.25:1.20, at least about 1:1.30:1.20, at least about 1:1.35:1.20, at least about 1:1.40:1.20, at least about 1:1.45:1.20, at least about 1:1.50:1.20, at least about 1:1.55:1.20, at least about 1:1.60:1.20, at least about 1:1.65:1.20, at least about 1:1.70:1.20, at least about 1:1.75:1.20, at least about 1:1.80:1.20, at least about 1:1.85:1.20, at least about 1:1.90:1.20, at least about 1:1.95:1.20, or at least about 1:2:1.20.

In a further embodiment, the ratio of PD-1 antagonist to TIGIT antagonist to CD96 antagonist may be at least about 1:0.25:1.25, at least about 1:0.30:1.25, at least about 1:0.35:1.25, at least about 1:0.40:1.25, at least about 1:0.45:1.25, at least about 1:0.50:1.25, at least about 1:0.55:1.25, at least about 1:0.60:1.25, at least about 1:0.65:1.25, at least about 1:0.70:1.25, at least about 1:0.75:1.25, at least about 1:0.80:1.25, at least about 1:0.85:1.25, at least about 1:0.90:1.25, at least about 1:0.95:1.25, at least about 1:1:1.25, at least about 1:1.05:1.25, at least about 1:1.10:1.25, at least about 1:1.15:1.25, at least about 1:1.20:1.25, at least about 1:1.25:1.25, at least about 1:1.30:1.25, at least about 1:1.35:1.25, at least about 1:1.40:1.25, at least about 1:1.45:1.25, at least about 1:1.50:1.25, at least about 1:1.55:1.25, at least about 1:1.60:1.25, at least about 1:1.65:1.25, at least about 1:1.70:1.25, at least about 1:1.75:1.25, at least about 1:1.80:1.25, at least about 1:1.85:1.25, at least about 1:1.90:1.25, at least about 1:1.95:1.25, or at least about 1:2:1.25.

In a further embodiment, the ratio of PD-1 antagonist to TIGIT antagonist to CD96 antagonist may be at least about 1:0.25:1.30, at least about 1:0.30:1.30, at least about 1:0.35:1.30, at least about 1:0.40:1.30, at least about 1:0.45:1.30, at least about 1:0.50:1.30, at least about 1:0.55:1.30, at least about 1:0.60:1.30, at least about 1:0.65:1.30, at least about 1:0.70:1.30, at least about 1:0.75:1.30, at least about 1:0.80:1.30, at least about 1:0.85:1.30, at least about 1:0.90:1.30, at least about 1:0.95:1.30, at least about 1:1:1.30, at least about 1:1.05:1.30, at least about 1:1.10:1.30, at least about 1:1.15:1.30, at least about 1:1.20:1.30, at least about 1:1.25:1.30, at least about 1:1.30:1.30, at least about 1:1.35:1.30, at least about 1:1.40:1.30, at least about 1:1.45:1.30, at least about 1:1.50:1.30, at least about 1:1.55:1.30, at least about 1:1.60:1.30, at least about 1:1.65:1.30, at least about 1:1.70:1.30, at least about 1:1.75:1.30, at least about 1:1.80:1.30, at least about 1:1.85:1.30, at least about 1:1.90:1.30, at least about 1:1.95:1.30, or at least about 1:2:1.30.

In a further embodiment, the ratio of PD-1 antagonist to TIGIT antagonist to CD96 antagonist may be at least about 1:0.25:1.35, at least about 1:0.30:1.35, at least about 1:0.35:1.35, at least about 1:0.40:1.35, at least about 1:0.45:1.35, at least about 1:0.50:1.35, at least about 1:0.55:1.35, at least about 1:0.60:1.35, at least about 1:0.65:1.35, at least about 1:0.70:1.35, at least about 1:0.75:1.35, at least about 1:0.80:1.35, at least about 1:0.85:1.35, at least about 1:0.90:1.35, at least about 1:0.95:1.35, at least about 1:1:1.35, at least about 1:1.05:1.35, at least about 1:1.10:1.35, at least about 1:1.15:1.35, at least about 1:1.20:1.35, at least about 1:1.25:1.35, at least about 1:1.30:1.35, at least about 1:1.35:1.35, at least about 1:1.40:1.35, at least about 1:1.45:1.35, at least about 1:1.50:1.35, at least about 1:1.55:1.35, at least about 1:1.60:1.35, at least about 1:1.65:1.35, at least about 1:1.70:1.35, at least about 1:1.75:1.35, at least about 1:1.80:1.35, at least about 1:1.85:1.35, at least about 1:1.90:1.35, at least about 1:1.95:1.35, or at least about 1:2:1.35.

In a further embodiment, the ratio of PD-1 antagonist to TIGIT antagonist to CD96 antagonist may be at least about 1:0.25:1.40, at least about 1:0.30:1.40, at least about 1:0.35:1.40, at least about 1:0.40:1.40, at least about 1:0.45:1.40, at least about 1:0.50:1.40, at least about 1:0.55:1.40, at least about 1:0.60:1.40, at least about 1:0.65:1.40, at least about 1:0.70:1.40, at least about 1:0.75:1.40, at least about 1:0.80:1.40, at least about 1:0.85:1.40, at least about 1:0.90:1.40, at least about 1:0.95:1.40, at least about 1:1:1.40, at least about 1:1.05:1.40, at least about 1:1.10:1.40, at least about 1:1.15:1.40, at least about 1:1.20:1.40, at least about 1:1.25:1.40, at least about 1:1.30:1.40, at least about 1:1.35:1.40, at least about 1:1.40:1.40, at least about 1:1.45:1.40, at least about 1:1.50:1.40, at least about 1:1.55:1.40, at least about 1:1.60:1.40, at least about 1:1.65:1.40, at least about 1:1.70:1.40, at least about 1:1.75:1.40, at least about 1:1.80:1.40, at least about 1:1.85:1.40, at least about 1:1.90:1.40, at least about 1:1.95:1.40, or at least about 1:2:1.40.

In a further embodiment, the ratio of PD-1 antagonist to TIGIT antagonist to CD96 antagonist may be at least about 1:0.25:1.45, at least about 1:0.30:1.45, at least about 1:0.35:1.45, at least about 1:0.40:1.45, at least about 1:0.45:1.45, at least about 1:0.50:1.45, at least about 1:0.55:1.45, at least about 1:0.60:1.45, at least about 1:0.65:1.45, at least about 1:0.70:1.45, at least about 1:0.75:1.45, at least about 1:0.80:1.45, at least about 1:0.85:1.45, at least about 1:0.90:1.45, at least about 1:0.95:1.45, at least about 1:1:1.45, at least about 1:1.05:1.45, at least about 1:1.10:1.45, at least about 1:1.15:1.45, at least about 1:1.20:1.45, at least about 1:1.25:1.45, at least about 1:1.30:1.45, at least about 1:1.35:1.45, at least about 1:1.40:1.45, at least about 1:1.45:1.45, at least about 1:1.50:1.45, at least about 1:1.55:1.45, at least about 1:1.60:1.45, at least about 1:1.65:1.45, at least about 1:1.70:1.45, at least about 1:1.75:1.45, at least about 1:1.80:1.45, at least about 1:1.85:1.45, at least about 1:1.90:1.45, at least about 1:1.95:1.45, or at least about 1:2:1.45.

In a further embodiment, the ratio of PD-1 antagonist to TIGIT antagonist to CD96 antagonist may be at least about 1:0.25:1.50, at least about 1:0.30:1.50, at least about 1:0.35:1.50, at least about 1:0.40:1.50, at least about 1:0.45:1.50, at least about 1:0.50:1.50, at least about 1:0.55:1.50, at least about 1:0.60:1.50, at least about 1:0.65:1.50, at least about 1:0.70:1.50, at least about 1:0.75:1.50, at least about 1:0.80:1.50, at least about 1:0.85:1.50, at least about 1:0.90:1.50, at least about 1:0.95:1.50, at least about 1:1:1.50, at least about 1:1.05:1.50, at least about 1:1.10:1.50, at least about 1:1.15:1.50, at least about 1:1.20:1.50, at least about 1:1.25:1.50, at least about 1:1.30:1.50, at least about 1:1.35:1.50, at least about 1:1.40:1.50, at least about 1:1.45:1.50, at least about 1:1.50:1.50, at least about 1:1.55:1.50, at least about 1:1.60:1.50, at least about 1:1.65:1.50, at least about 1:1.70:1.50, at least about 1:1.75:1.50, at least about 1:1.80:1.50, at least about 1:1.85:1.50, at least about 1:1.90:1.50, at least about 1:1.95:1.50, or at least about 1:2:1.50.

In a further embodiment, the ratio of PD-1 antagonist to TIGIT antagonist to CD96 antagonist may be at least about 1:0.25:1.55, at least about 1:0.30:1.55, at least about 1:0.35:1.55, at least about 1:0.40:1.55, at least about 1:0.45:1.55, at least about 1:0.50:1.55, at least about 1:0.55:1.55, at least about 1:0.60:1.55, at least about 1:0.65:1.55, at least about 1:0.70:1.55, at least about 1:0.75:1.55, at least about 1:0.80:1.55, at least about 1:0.85:1.55, at least about 1:0.90:1.55, at least about 1:0.95:1.55, at least about 1:1:1.55, at least about 1:1.05:1.55, at least about 1:1.10:1.55, at least about 1:1.15:1.55, at least about 1:1.20:1.55, at least about 1:1.25:1.55, at least about 1:1.30:1.55, at least about 1:1.35:1.55, at least about 1:1.40:1.55, at least about 1:1.45:1.55, at least about 1:1.50:1.55, at least about 1:1.55:1.55, at least about 1:1.60:1.55, at least about 1:1.65:1.55, at least about 1:1.70:1.55, at least about 1:1.75:1.55, at least about 1:1.80:1.55, at least about 1:1.85:1.55, at least about 1:1.90:1.55, at least about 1:1.95:1.55, or at least about 1:2:1.55.

In a further embodiment, the ratio of PD-1 antagonist to TIGIT antagonist to CD96 antagonist may be at least about 1:0.25:1.60, at least about 1:0.30:1.60, at least about 1:0.35:1.60, at least about 1:0.40:1.60, at least about 1:0.45:1.60, at least about 1:0.50:1.60, at least about 1:0.55:1.60, at least about 1:0.60:1.60, at least about 1:0.65:1.60, at least about 1:0.70:1.60, at least about 1:0.75:1.60, at least about 1:0.80:1.60, at least about 1:0.85:1.60, at least about 1:0.90:1.60, at least about 1:0.95:1.60, at least about 1:1:1.60, at least about 1:1.05:1.60, at least about 1:1.10:1.60, at least about 1:1.15:1.60, at least about 1:1.20:1.60, at least about 1:1.25:1.60, at least about 1:1.30:1.60, at least about 1:1.35:1.60, at least about 1:1.40:1.60, at least about 1:1.45:1.60, at least about 1:1.50:1.60, at least about 1:1.55:1.60, at least about 1:1.60:1.60, at least about 1:1.65:1.60, at least about 1:1.70:1.60, at least about 1:1.75:1.60, at least about 1:1.80:1.60, at least about 1:1.85:1.60, at least about 1:1.90:1.60, at least about 1:1.95:1.60, or at least about 1:2:1.60.

In a further embodiment, the ratio of PD-1 antagonist to TIGIT antagonist to CD96 antagonist may be at least about 1:0.25:1.65, at least about 1:0.30:1.65, at least about 1:0.35:1.65, at least about 1:0.40:1.65, at least about 1:0.45:1.65, at least about 1:0.50:1.65, at least about 1:0.55:1.65, at least about 1:0.60:1.65, at least about 1:0.65:1.65, at least about 1:0.70:1.65, at least about 1:0.75:1.65, at least about 1:0.80:1.65, at least about 1:0.85:1.65, at least about 1:0.90:1.65, at least about 1:0.95:1.65, at least about 1:1:1.65, at least about 1:1.05:1.65, at least about 1:1.10:1.65, at least about 1:1.15:1.65, at least about 1:1.20:1.65, at least about 1:1.25:1.65, at least about 1:1.30:1.65, at least about 1:1.35:1.65, at least about 1:1.40:1.65, at least about 1:1.45:1.65, at least about 1:1.50:1.65, at least about 1:1.55:1.65, at least about 1:1.60:1.65, at least about 1:1.65:1.65, at least about 1:1.70:1.65, at least about 1:1.75:1.65, at least about 1:1.80:1.65, at least about 1:1.85:1.65, at least about 1:1.90:1.65, at least about 1:1.95:1.65, or at least about 1:2:1.65.

In a further embodiment, the ratio of PD-1 antagonist to TIGIT antagonist to CD96 antagonist may be at least about 1:0.25:1.70, at least about 1:0.30:1.70, at least about 1:0.35:1.70, at least about 1:0.40:1.70, at least about 1:0.45:1.70, at least about 1:0.50:1.70, at least about 1:0.55:1.70, at least about 1:0.60:1.70, at least about 1:0.65:1.70, at least about 1:0.70:1.70, at least about 1:0.75:1.70, at least about 1:0.80:1.70, at least about 1:0.85:1.70, at least about 1:0.90:1.70, at least about 1:0.95:1.70, at least about 1:1:1.70, at least about 1:1.05:1.70, at least about 1:1.10:1.70, at least about 1:1.15:1.70, at least about 1:1.20:1.70, at least about 1:1.25:1.70, at least about 1:1.30:1.70, at least about 1:1.35:1.70, at least about 1:1.40:1.70, at least about 1:1.45:1.70, at least about 1:1.50:1.70, at least about 1:1.55:1.70, at least about 1:1.60:1.70, at least about 1:1.65:1.70, at least about 1:1.70:1.70, at least about 1:1.75:1.70, at least about 1:1.80:1.70, at least about 1:1.85:1.70, at least about 1:1.90:1.70, at least about 1:1.95:1.70, or at least about 1:2:1.70.

In a further embodiment, the ratio of PD-1 antagonist to TIGIT antagonist to CD96 antagonist may be at least about 1:0.25:1.75, at least about 1:0.30:1.75, at least about 1:0.35:1.75, at least about 1:0.40:1.75, at least about 1:0.45:1.75, at least about 1:0.50:1.75, at least about 1:0.55:1.75, at least about 1:0.60:1.75, at least about 1:0.65:1.75, at least about 1:0.70:1.75, at least about 1:0.75:1.75, at least about 1:0.80:1.75, at least about 1:0.85:1.75, at least about 1:0.90:1.75, at least about 1:0.95:1.75, at least about 1:1:1.75, at least about 1:1.05:1.75, at least about 1:1.10:1.75, at least about 1:1.15:1.75, at least about 1:1.20:1.75, at least about 1:1.25:1.75, at least about 1:1.30:1.75, at least about 1:1.35:1.75, at least about 1:1.40:1.75, at least about 1:1.45:1.75, at least about 1:1.50:1.75, at least about 1:1.55:1.75, at least about 1:1.60:1.75, at least about 1:1.65:1.75, at least about 1:1.70:1.75, at least about 1:1.75:1.75, at least about 1:1.80:1.75, at least about 1:1.85:1.75, at least about 1:1.90:1.75, at least about 1:1.95:1.75, or at least about 1:2:1.75.

In a further embodiment, the ratio of PD-1 antagonist to TIGIT antagonist to CD96 antagonist may be at least about 1:0.25:1.80, at least about 1:0.30:1.80, at least about 1:0.35:1.80, at least about 1:0.40:1.80, at least about 1:0.45:1.80, at least about 1:0.50:1.80, at least about 1:0.55:1.80, at least about 1:0.60:1.80, at least about 1:0.65:1.80, at least about 1:0.70:1.80, at least about 1:0.75:1.80, at least about 1:0.80:1.80, at least about 1:0.85:1.80, at least about 1:0.90:1.80, at least about 1:0.95:1.80, at least about 1:1:1.80, at least about 1:1.05:1.80, at least about 1:1.10:1.80, at least about 1:1.15:1.80, at least about 1:1.20:1.80, at least about 1:1.25:1.80, at least about 1:1.30:1.80, at least about 1:1.35:1.80, at least about 1:1.40:1.80, at least about 1:1.45:1.80, at least about 1:1.50:1.80, at least about 1:1.55:1.80, at least about 1:1.60:1.80, at least about 1:1.65:1.80, at least about 1:1.70:1.80, at least about 1:1.75:1.80, at least about 1:1.80:1.80, at least about 1:1.85:1.80, at least about 1:1.90:1.80, at least about 1:1.95:1.80, or at least about 1:2:1.80.

In a further embodiment, the ratio of PD-1 antagonist to TIGIT antagonist to CD96 antagonist may be at least about 1:0.25:1.85, at least about 1:0.30:1.85, at least about 1:0.35:1.85, at least about 1:0.40:1.85, at least about 1:0.45:1.85, at least about 1:0.50:1.85, at least about 1:0.55:1.85, at least about 1:0.60:1.85, at least about 1:0.65:1.85, at least about 1:0.70:1.85, at least about 1:0.75:1.85, at least about 1:0.80:1.85, at least about 1:0.85:1.85, at least about 1:0.90:1.85, at least about 1:0.95:1.85, at least about 1:1:1.85, at least about 1:1.05:1.85, at least about 1:1.10:1.85, at least about 1:1.15:1.85, at least about 1:1.20:1.85, at least about 1:1.25:1.85, at least about 1:1.30:1.85, at least about 1:1.35:1.85, at least about 1:1.40:1.85, at least about 1:1.45:1.85, at least about 1:1.50:1.85, at least about 1:1.55:1.85, at least about 1:1.60:1.85, at least about 1:1.65:1.85, at least about 1:1.70:1.85, at least about 1:1.75:1.85, at least about 1:1.80:1.85, at least about 1:1.85:1.85, at least about 1:1.90:1.85, at least about 1:1.95:1.85, or at least about 1:2:1.85.

In a further embodiment, the ratio of PD-1 antagonist to TIGIT antagonist to CD96 antagonist may be at least about 1:0.25:1.90, at least about 1:0.30:1.90, at least about 1:0.35:1.90, at least about 1:0.40:1.90, at least about 1:0.45:1.90, at least about 1:0.50:1.90, at least about 1:0.55:1.90, at least about 1:0.60:1.90, at least about 1:0.65:1.90, at least about 1:0.70:1.90, at least about 1:0.75:1.90, at least about 1:0.80:1.90, at least about 1:0.85:1.90, at least about 1:0.90:1.90, at least about 1:0.95:1.90, at least about 1:1:1.90, at least about 1:1.05:1.90, at least about 1:1.10:1.90, at least about 1:1.15:1.90, at least about 1:1.20:1.90, at least about 1:1.25:1.90, at least about 1:1.30:1.90, at least about 1:1.35:1.90, at least about 1:1.40:1.90, at least about 1:1.45:1.90, at least about 1:1.50:1.90, at least about 1:1.55:1.90, at least about 1:1.60:1.90, at least about 1:1.65:1.90, at least about 1:1.70:1.90, at least about 1:1.75:1.90, at least about 1:1.80:1.90, at least about 1:1.85:1.90, at least about 1:1.90:1.90, at least about 1:1.95:1.90, or at least about 1:2:1.90.

In a further embodiment, the ratio of PD-1 antagonist to TIGIT antagonist to CD96 antagonist may be at least about 1:0.25:1.95, at least about 1:0.30:1.95, at least about 1:0.35:1.95, at least about 1:0.40:1.95, at least about 1:0.45:1.95, at least about 1:0.50:1.95, at least about 1:0.55:1.95, at least about 1:0.60:1.95, at least about 1:0.65:1.95, at least about 1:0.70:1.95, at least about 1:0.75:1.95, at least about 1:0.80:1.95, at least about 1:0.85:1.95, at least about 1:0.90:1.95, at least about 1:0.95:1.95, at least about 1:1:1.95, at least about 1:1.05:1.95, at least about 1:1.10:1.95, at least about 1:1.15:1.95, at least about 1:1.20:1.95, at least about 1:1.25:1.95, at least about 1:1.30:1.95, at least about 1:1.35:1.95, at least about 1:1.40:1.95, at least about 1:1.45:1.95, at least about 1:1.50:1.95, at least about 1:1.55:1.95, at least about 1:1.60:1.95, at least about 1:1.65:1.95, at least about 1:1.70:1.95, at least about 1:1.75:1.95, at least about 1:1.80:1.95, at least about 1:1.85:1.95, at least about 1:1.90:1.95, at least about 1:1.95:1.95, or at least about 1:2:1.95.

In a further embodiment, the ratio of PD-1 antagonist to TIGIT antagonist to CD96 antagonist may be at least about 1:0.25:2, at least about 1:0.30:2, at least about 1:0.35:2, at least about 1:0.40:2, at least about 1:0.45:2, at least about 1:0.50:2, at least about 1:0.55:2, at least about 1:0.60:2, at least about 1:0.65:2, at least about 1:0.70:2, at least about 1:0.75:2, at least about 1:0.80:2, at least about 1:0.85:2, at least about 1:0.90:2, at least about 1:0.95:2, at least about 1:1:2, at least about 1:1.05:2, at least about 1:1.10:2, at least about 1:1.15:2, at least about 1:1.20:2, at least about 1:1.25:2, at least about 1:1.30:2, at least about 1:1.35:2, at least about 1:1.40:2, at least about 1:1.45:2, at least about 1:1.50:2, at least about 1:1.55:2, at least about 1:1.60:2, at least about 1:1.65:2, at least about 1:1.70:2, at least about 1:1.75:2, at least about 1:1.80:2, at least about 1:1.85:2, at least about 1:1.90:2, at least about 1:1.95:2, or at least about 1:2:2.

In a further embodiment, the ratio of PD-1 antagonist to PVRIG antagonist to CD96 antagonist may be at least about 1:0.25:0.25, at least about 1:0.30:0.30, at least about 1:0.35:0.35, at least about 1:0.40:0.40, at least about 1:0.45:0.45, at least about 1:0.50:0.50, at least about 1:0.55:0.55, at least about 1:0.60:0.60, at least about 1:0.65:0.65, at least about 1:0.70:0.70, at least about 1:0.75:0.75, at least about 1:0.80:0.80, at least about 1:0.85:0.85, at least about 1:0.90:0.90, at least about 1:0.95:0.95, at least about 1:1:1, at least about 1:1.05:1.05, at least about 1:1.10:1.10, at least about 1:1.15:1.15, at least about 1:1.20:1.20, at least about 1:1.25:1.25, at least about 1:1.30:1.30, at least about 1:1.35:1.35, at least about 1:1.40:1.40, at least about 1:1.45:1.45, at least about 1:1.50:1.50, at least about 1:1.55:1.55, at least about 1:1.60:1.60, at least about 1:1.65:1.65, at least about 1:1.70:1.70, at least about 1:1.75:1.75, at least about 1:1.80:1.80, at least about 1:1.85:1.85, at least about 1:1.90:1.90, at least about 1:1.95:1.95, or at least about 1:2:2.

In a further embodiment, the ratio of PD-1 antagonist to PVRIG antagonist to CD96 antagonist may be at least about 1:0.25:0.30, at least about 1:0.25:0.35, at least about 1:0.25:0.40, at least about 1:0.25:0.45, at least about 1:0.25:0.50, at least about 1:0.25:0.55, at least about 1:0.25:0.60, at least about 1:0.25:0.65, at least about 1:0.25:0.70, at least about 1:0.25:0.75, at least about 1:0.25:0.80, at least about 1:0.25:0.85, at least about 1:0.25:0.90, at least about 1:0.25:0.95, at least about 1:0.25:1, at least about 1:0.25:1.05, at least about 1:0.25:1.10, at least about 1:0.25:1.15, at least about 1:0.25:1.20, at least about 1:0.25:1.25, at least about 1:0.25:1.30, at least about 1:0.25:1.35, at least about 1:0.25:1.40, at least about 1:0.25:1.45, at least about 1:0.25:1.50, at least about 1:0.25:1.55, at least about 1:0.25:1.60, at least about 1:0.25:1.65, at least about 1:0.25:1.70, at least about 1:0.25:1.75, at least about 1:0.25:1.80, at least about 1:0.25:1.85, at least about 1:0.25:1.90, at least about 1:0.25:1.95, or at least about 1:0.25:2.

In a further embodiment, the ratio of PD-1 antagonist to PVRIG antagonist to CD96 antagonist may be at least about 1:0.30:0.25, at least about 1:0.30:0.30, at least about 1:0.30:0.35, at least about 1:0.30:0.40, at least about 1:0.30:0.45, at least about 1:0.30:0.50, at least about 1:0.30:0.55, at least about 1:0.30:0.60, at least about 1:0.30:0.65, at least about 1:0.30:0.70, at least about 1:0.30:0.75, at least about 1:0.30:0.80, at least about 1:0.30:0.85, at least about 1:0.30:0.90, at least about 1:0.30:0.95, at least about 1:0.30:1, at least about 1:0.30:1.05, at least about 1:0.30:1.10, at least about 1:0.30:1.15, at least about 1:0.30:1.20, at least about 1:0.30:1.25, at least about 1:0.30:1.30, at least about 1:0.30:1.35, at least about 1:0.30:1.40, at least about 1:0.30:1.45, at least about 1:0.30:1.50, at least about 1:0.30:1.55, at least about 1:0.30:1.60, at least about 1:0.30:1.65, at least about 1:0.30:1.70, at least about 1:0.30:1.75, at least about 1:0.30:1.80, at least about 1:0.30:1.85, at least about 1:0.30:1.90, at least about 1:0.30:1.95, or at least about 1:0.30:2.

In a further embodiment, the ratio of PD-1 antagonist to PVRIG antagonist to CD96 antagonist may be at least about 1:0.35:0.25, at least about 1:0.35:0.30, at least about 1:0.35:0.35, at least about 1:0.35:0.40, at least about 1:0.35:0.45, at least about 1:0.35:0.50, at least about 1:0.35:0.55, at least about 1:0.35:0.60, at least about 1:0.35:0.65, at least about 1:0.35:0.70, at least about 1:0.35:0.75, at least about 1:0.35:0.80, at least about 1:0.35:0.85, at least about 1:0.35:0.90, at least about 1:0.35:0.95, at least about 1:0.35:1, at least about 1:0.35:1.05, at least about 1:0.35:1.10, at least about 1:0.35:1.15, at least about 1:0.35:1.20, at least about 1:0.35:1.25, at least about 1:0.35:1.30, at least about 1:0.35:1.35, at least about 1:0.35:1.40, at least about 1:0.35:1.45, at least about 1:0.35:1.50, at least about 1:0.35:1.55, at least about 1:0.35:1.60, at least about 1:0.35:1.65, at least about 1:0.35:1.70, at least about 1:0.35:1.75, at least about 1:0.35:1.80, at least about 1:0.35:1.85, at least about 1:0.35:1.90, at least about 1:0.35:1.95, or at least about 1:0.35:2.

In a further embodiment, the ratio of PD-1 antagonist to PVRIG antagonist to CD96 antagonist may be at least about 1:0.40:0.25, at least about 1:0.40:0.30, at least about 1:0.40:0.35, at least about 1:0.40:0.40, at least about 1:0.40:0.45, at least about 1:0.40:0.50, at least about 1:0.40:0.55, at least about 1:0.40:0.60, at least about 1:0.40:0.65, at least about 1:0.40:0.70, at least about 1:0.40:0.75, at least about 1:0.40:0.80, at least about 1:0.40:0.85, at least about 1:0.40:0.90, at least about 1:0.40:0.95, at least about 1:0.40:1, at least about 1:0.40:1.05, at least about 1:0.40:1.10, at least about 1:0.40:1.15, at least about 1:0.40:1.20, at least about 1:0.40:1.25, at least about 1:0.40:1.30, at least about 1:0.40:1.35, at least about 1:0.40:1.40, at least about 1:0.40:1.45, at least about 1:0.40:1.50, at least about 1:0.40:1.55, at least about 1:0.40:1.60, at least about 1:0.40:1.65, at least about 1:0.40:1.70, at least about 1:0.40:1.75, at least about 1:0.40:1.80, at least about 1:0.40:1.85, at least about 1:0.40:1.90, at least about 1:0.40:1.95, or at least about 1:0.40:2.

In a further embodiment, the ratio of PD-1 antagonist to PVRIG antagonist to CD96 antagonist may be at least about 1:0.45:0.25, at least about 1:0.45:0.30, at least about 1:0.45:0.35, at least about 1:0.45:0.40, at least about 1:0.45:0.45, at least about 1:0.45:0.50, at least about 1:0.45:0.55, at least about 1:0.45:0.60, at least about 1:0.45:0.65, at least about 1:0.45:0.70, at least about 1:0.45:0.75, at least about 1:0.45:0.80, at least about 1:0.45:0.85, at least about 1:0.45:0.90, at least about 1:0.45:0.95, at least about 1:0.45:1, at least about 1:0.45:1.05, at least about 1:0.45:1.10, at least about 1:0.45:1.15, at least about 1:0.45:1.20, at least about 1:0.45:1.25, at least about 1:0.45:1.30, at least about 1:0.45:1.35, at least about 1:0.45:1.40, at least about 1:0.45:1.45, at least about 1:0.45:1.50, at least about 1:0.45:1.55, at least about 1:0.45:1.60, at least about 1:0.45:1.65, at least about 1:0.45:1.70, at least about 1:0.45:1.75, at least about 1:0.45:1.80, at least about 1:0.45:1.85, at least about 1:0.45:1.90, at least about 1:0.45:1.95, or at least about 1:0.45:2.

In a further embodiment, the ratio of PD-1 antagonist to PVRIG antagonist to CD96 antagonist may be at least about 1:0.50:0.25, at least about 1:0.50:0.30, at least about 1:0.50:0.35, at least about 1:0.50:0.40, at least about 1:0.50:0.45, at least about 1:0.50:0.50, at least about 1:0.50:0.55, at least about 1:0.50:0.60, at least about 1:0.50:0.65, at least about 1:0.50:0.70, at least about 1:0.50:0.75, at least about 1:0.50:0.80, at least about 1:0.50:0.85, at least about 1:0.50:0.90, at least about 1:0.50:0.95, at least about 1:0.50:1, at least about 1:0.50:1.05, at least about 1:0.50:1.10, at least about 1:0.50:1.15, at least about 1:0.50:1.20, at least about 1:0.50:1.25, at least about 1:0.50:1.30, at least about 1:0.50:1.35, at least about 1:0.50:1.40, at least about 1:0.50:1.45, at least about 1:0.50:1.50, at least about 1:0.50:1.55, at least about 1:0.50:1.60, at least about 1:0.50:1.65, at least about 1:0.50:1.70, at least about 1:0.50:1.75, at least about 1:0.50:1.80, at least about 1:0.50:1.85, at least about 1:0.50:1.90, at least about 1:0.50:1.95, or at least about 1:0.50:2.

In a further embodiment, the ratio of PD-1 antagonist to PVRIG antagonist to CD96 antagonist may be at least about 1:0.55:0.25, at least about 1:0.55:0.30, at least about 1:0.55:0.35, at least about 1:0.55:0.40, at least about 1:0.55:0.45, at least about 1:0.55:0.50, at least about 1:0.55:0.55, at least about 1:0.55:0.60, at least about 1:0.55:0.65, at least about 1:0.55:0.70, at least about 1:0.55:0.75, at least about 1:0.55:0.80, at least about 1:0.55:0.85, at least about 1:0.55:0.90, at least about 1:0.55:0.95, at least about 1:0.55:1, at least about 1:0.55:1.05, at least about 1:0.55:1.10, at least about 1:0.55:1.15, at least about 1:0.55:1.20, at least about 1:0.55:1.25, at least about 1:0.55:1.30, at least about 1:0.55:1.35, at least about 1:0.55:1.40, at least about 1:0.55:1.45, at least about 1:0.55:1.50, at least about 1:0.55:1.55, at least about 1:0.55:1.60, at least about 1:0.55:1.65, at least about 1:0.55:1.70, at least about 1:0.55:1.75, at least about 1:0.55:1.80, at least about 1:0.55:1.85, at least about 1:0.55:1.90, 1:0.55:1.95, or at least about 1:0.55:2.

In a further embodiment, the ratio of PD-1 antagonist to PVRIG antagonist to CD96 antagonist may be at least about 1:0.60:0.25, at least about 1:0.60:0.30, at least about 1:0.60:0.35, at least about 1:0.60:0.40, at least about 1:0.60:0.45, at least about 1:0.60:0.50, at least about 1:0.60:0.55, at least about 1:0.60:0.60, at least about 1:0.60:0.65, at least about 1:0.60:0.70, at least about 1:0.60:0.75, at least about 1:0.60:0.80, at least about 1:0.60:0.85, at least about 1:0.60:0.90, at least about 1:0.60:0.95, at least about 1:0.60:1, at least about 1:0.60:1.05, at least about 1:0.60:1.10, at least about 1:0.60:1.15, at least about 1:0.60:1.20, at least about 1:0.60:1.25, at least about 1:0.60:1.30, at least about 1:0.60:1.35, at least about 1:0.60:1.40, at least about 1:0.60:1.45, at least about 1:0.60:1.50, at least about 1:0.60:1.55, at least about 1:0.60:1.60, at least about 1:0.60:1.65, at least about 1:0.60:1.70, at least about 1:0.60:1.75, at least about 1:0.60:1.80, at least about 1:0.60:1.85, at least about 1:0.60:1.90, at least about 1:0.60:1.95, or at least about 1:0.60:2.

In a further embodiment, the ratio of PD-1 antagonist to PVRIG antagonist to CD96 antagonist may be at least about 1:0.65:0.25, at least about 1:0.65:0.30, at least about 1:0.65:0.35, at least about 1:0.65:0.40, at least about 1:0.65:0.45, at least about 1:0.65:0.50, at least about 1:0.65:0.55, at least about 1:0.65:0.60, at least about 1:0.65:0.65, at least about 1:0.65:0.70, at least about 1:0.65:0.75, at least about 1:0.65:0.80, at least about 1:0.65:0.85, at least about 1:0.65:0.90, at least about 1:0.65:0.95, at least about 1:0.65:1, at least about 1:0.65:1.05, at least about 1:0.65:1.10, at least about 1:0.65:1.15, at least about 1:0.65:1.20, at least about 1:0.65:1.25, at least about 1:0.65:1.30, at least about 1:0.65:1.35, at least about 1:0.65:1.40, at least about 1:0.65:1.45, at least about 1:0.65:1.50, at least about 1:0.65:1.55, at least about 1:0.65:1.60, at least about 1:0.65:1.65, at least about 1:0.65:1.70, at least about 1:0.65:1.75, at least about 1:0.65:1.80, at least about 1:0.65:1.85, at least about 1:0.65:1.90, at least about 1:0.65:1.95, or at least about 1:0.65:2.

In a further embodiment, the ratio of PD-1 antagonist to PVRIG antagonist to CD96 antagonist may be at least about 1:0.70:0.25, at least about 1:0.70:0.30, at least about 1:0.70:0.35, at least about 1:0.70:0.40, at least about 1:0.70:0.45, at least about 1:0.70:0.50, at least about 1:0.70:0.55, at least about 1:0.70:0.60, at least about 1:0.70:0.65, at least about 1:0.70:0.70, at least about 1:0.70:0.75, at least about 1:0.70:0.80, at least about 1:0.70:0.85, at least about 1:0.70:0.90, at least about 1:0.70:0.95, at least about 1:0.70:1, at least about 1:0.70:1.05, at least about 1:0.70:1.10, at least about 1:0.70:1.15, at least about 1:0.70:1.20, at least about 1:0.70:1.25, at least about 1:0.70:1.30, at least about 1:0.70:1.35, at least about 1:0.70:1.40, at least about 1:0.70:1.45, at least about 1:0.70:1.50, at least about 1:0.70:1.55, at least about 1:0.70:1.60, at least about 1:0.70:1.65, at least about 1:0.70:1.70, at least about 1:0.70:1.75, at least about 1:0.70:1.80, at least about 1:0.70:1.85, at least about 1:0.70:1.90, at least about 1:0.70:1.95, or at least about 1:0.70:2.

In a further embodiment, the ratio of PD-1 antagonist to PVRIG antagonist to CD96 antagonist may be at least about 1:0.75:0.25, at least about 1:0.75:0.30, at least about 1:0.75:0.35, at least about 1:0.75:0.40, at least about 1:0.75:0.45, at least about 1:0.75:0.50, at least about 1:0.75:0.55, at least about 1:0.75:0.60, at least about 1:0.75:0.65, at least about 1:0.75:0.70, at least about 1:0.75:0.75, at least about 1:0.75:0.80, at least about 1:0.75:0.85, at least about 1:0.75:0.90, at least about 1:0.75:0.95, at least about 1:0.75:1, at least about 1:0.75:1.05, at least about 1:0.75:1.10, at least about 1:0.75:1.15, at least about 1:0.75:1.20, at least about 1:0.75:1.25, at least about 1:0.75:1.30, at least about 1:0.75:1.35, at least about 1:0.75:1.40, at least about 1:0.75:1.45, at least about 1:0.75:1.50, at least about 1:0.75:1.55, at least about 1:0.75:1.60, at least about 1:0.75:1.65, at least about 1:0.75:1.70, at least about 1:0.75:1.75, at least about 1:0.75:1.80, at least about 1:0.75:1.85, at least about 1:0.75:1.90, at least about 1:0.75:1.95, or at least about 1:0.75:2.

In a further embodiment, the ratio of PD-1 antagonist to PVRIG antagonist to CD96 antagonist may be at least about 1:0.80:0.25, at least about 1:0.80:0.30, at least about 1:0.80:0.35, at least about 1:0.80:0.40, at least about 1:0.80:0.45, at least about 1:0.80:0.50, at least about 1:0.80:0.55, at least about 1:0.80:0.60, at least about 1:0.80:0.65, at least about 1:0.80:0.70, at least about 1:0.80:0.75, at least about 1:0.80:0.80, at least about 1:0.80:0.85, at least about 1:0.80:0.90, at least about 1:0.80:0.95, at least about 1:0.80:1, at least about 1:0.80:1.05, at least about 1:0.80:1.10, at least about 1:0.80:1.15, at least about 1:0.80:1.20, at least about 1:0.80:1.25, at least about 1:0.80:1.30, at least about 1:0.80:1.35, at least about 1:0.80:1.40, at least about 1:0.80:1.45, at least about 1:0.80:1.50, at least about 1:0.80:1.55, at least about 1:0.80:1.60, at least about 1:0.80:1.65, at least about 1:0.80:1.70, at least about 1:0.80:1.75, at least about 1:0.80:1.80, at least about 1:0.80:1.85, at least about 1:0.80:1.90, at least about 1:0.80:1.95, or at least about 1:0.80:2.

In a further embodiment, the ratio of PD-1 antagonist to PVRIG antagonist to CD96 antagonist may be at least about 1:0.85:0.25, at least about 1:0.85:0.30, at least about 1:0.85:0.35, at least about 1:0.85:0.40, at least about 1:0.85:0.45, at least about 1:0.85:0.50, at least about 1:0.85:0.55, at least about 1:0.85:0.60, at least about 1:0.85:0.65, at least about 1:0.85:0.70, at least about 1:0.85:0.75, at least about 1:0.85:0.80, at least about 1:0.85:0.85, at least about 1:0.85:0.90, at least about 1:0.85:0.95, at least about 1:0.85:1, at least about 1:0.85:1.05, at least about 1:0.85:1.10, at least about 1:0.85:1.15, at least about 1:0.85:1.20, at least about 1:0.85:1.25, at least about 1:0.85:1.30, at least about 1:0.85:1.35, at least about 1:0.85:1.40, at least about 1:0.85:1.45, at least about 1:0.85:1.50, at least about 1:0.85:1.55, at least about 1:0.85:1.60, at least about 1:0.85:1.65, at least about 1:0.85:1.70, at least about 1:0.85:1.75, at least about 1:0.85:1.80, at least about 1:0.85:1.85, at least about 1:0.85:1.90, at least about 1:0.85:1.95, or at least about 1:0.85:2.

In a further embodiment, the ratio of PD-1 antagonist to PVRIG antagonist to CD96 antagonist may be at least about 1:0.90:0.25, at least about 1:0.90:0.30, at least about 1:0.90:0.35, at least about 1:0.90:0.40, at least about 1:0.90:0.45, at least about 1:0.90:0.50, at least about 1:0.90:0.55, at least about 1:0.90:0.60, at least about 1:0.90:0.65, at least about 1:0.90:0.70, at least about 1:0.90:0.75, at least about 1:0.90:0.80, at least about 1:0.90:0.85, at least about 1:0.90:0.90, at least about 1:0.90:0.95, at least about 1:0.90:1, at least about 1:0.90:1.05, at least about 1:0.90:1.10, at least about 1:0.90:1.15, at least about 1:0.90:1.20, at least about 1:0.90:1.25, at least about 1:0.90:1.30, at least about 1:0.90:1.35, at least about 1:0.90:1.40, at least about 1:0.90:1.45, at least about 1:0.90:1.50, at least about 1:0.90:1.55, at least about 1:0.90:1.60, at least about 1:0.90:1.65, at least about 1:0.90:1.70, at least about 1:0.90:1.75, at least about 1:0.90:1.80, at least about 1:0.90:1.85, at least about 1:0.90:1.90, at least about 1:0.90:1.95, or at least about 1:0.90:2.

In a further embodiment, the ratio of PD-1 antagonist to PVRIG antagonist to CD96 antagonist may be at least about 1:0.95:0.25, at least about 1:0.95:0.30, at least about 1:0.95:0.35, at least about 1:0.95:0.40, at least about 1:0.95:0.45, at least about 1:0.95:0.50, at least about 1:0.95:0.55, at least about 1:0.95:0.60, at least about 1:0.95:0.65, at least about 1:0.95:0.70, at least about 1:0.95:0.75, at least about 1:0.95:0.80, at least about 1:0.95:0.85, at least about 1:0.95:0.90, at least about 1:0.95:0.95, at least about 1:0.95:1, at least about 1:0.95:1.05, at least about 1:0.95:1.10, at least about 1:0.95:1.15, at least about 1:0.95:1.20, at least about 1:0.95:1.25, at least about 1:0.95:1.30, at least about 1:0.95:1.35, at least about 1:0.95:1.40, at least about 1:0.95:1.45, at least about 1:0.95:1.50, at least about 1:0.95:1.55, at least about 1:0.95:1.60, at least about 1:0.95:1.65, at least about 1:0.95:1.70, at least about 1:0.95:1.75, at least about 1:0.95:1.80, at least about 1:0.95:1.85, at least about 1:0.95:1.90, at least about 1:0.95:1.95, or at least about 1:0.95:2.

In a further embodiment, the ratio of PD-1 antagonist to PVRIG antagonist to CD96 antagonist may be at least about 1:1:0.25, at least about 1:1:0.30, at least about 1:1:0.35, at least about 1:1:0.40, at least about 1:1:0.45, at least about 1:1:0.50, at least about 1:1:0.55, at least about 1:1:0.60, at least about 1:1:0.65, at least about 1:1:0.70, at least about 1:1:0.75, at least about 1:1:0.80, at least about 1:1:0.85, at least about 1:1:0.90, at least about 1:1:0.95, at least about 1:1:1, at least about 1:1:1.05, at least about 1:1:1.10, at least about 1:1:1.15, at least about 1:1:1.20, at least about 1:1:1.25, at least about 1:1:1.30, at least about 1:1:1.35, at least about 1:1:1.40, at least about 1:1:1.45, at least about 1:1:1.50, at least about 1:1:1.55, at least about 1:1:1.60, at least about 1:1:1.65, at least about 1:1:1.70, at least about 1:1:1.75, at least about 1:1:1.80, at least about 1:1:1.85, at least about 1:1:1.90, at least about 1:1:1.95, or at least about 1:1:2.

In a further embodiment, the ratio of PD-1 antagonist to PVRIG antagonist to CD96 antagonist may be at least about 1:1.05:0.25, at least about 1:1.05:0.30, at least about 1:1.05:0.35, at least about 1:1.05:0.40, at least about 1:1.05:0.45, at least about 1:1.05:0.50, at least about 1:1.05:0.55, at least about 1:1.05:0.60, at least about 1:1.05:0.65, at least about 1:1.05:0.70, at least about 1:1.05:0.75, at least about 1:1.05:0.80, at least about 1:1.05:0.85, at least about 1:1.05:0.90, at least about 1:1.05:0.95, at least about 1:1.05:1, at least about 1:1.05:1.05, at least about 1:1.05:1.10, at least about 1:1.05:1.15, at least about 1:1.05:1.20, at least about 1:1.05:1.25, at least about 1:1.05:1.30, at least about 1:1.05:1.35, at least about 1:1.05:1.40, at least about 1:1.05:1.45, at least about 1:1.05:1.50, at least about 1:1.05:1.55, at least about 1:1.05:1.60, at least about 1:1.05:1.65, at least about 1:1.05:1.70, at least about 1:1.05:1.75, at least about 1:1.05:1.80, at least about 1:1.05:1.85, at least about 1:1.05:1.90, at least about 1:1.05:1.95, or at least about 1:1.05:2.

In a further embodiment, the ratio of PD-1 antagonist to PVRIG antagonist to CD96 antagonist may be at least about 1:1.10:0.25, at least about 1:1.10:0.30, at least about 1:1.10:0.35, at least about 1:1.10:0.40, at least about 1:1.10:0.45, at least about 1:1.10:0.50, at least about 1:1.10:0.55, at least about 1:1.10:0.60, at least about 1:1.10:0.65, at least about 1:1.10:0.70, at least about 1:1.10:0.75, at least about 1:1.10:0.80, at least about 1:1.10:0.85, at least about 1:1.10:0.90, at least about 1:1.10:0.95, at least about 1:1.10:1, at least about 1:1.10:1.05, at least about 1:1.10:1.10, at least about 1:1.10:1.15, at least about 1:1.10:1.20, at least about 1:1.10:1.25, at least about 1:1.10:1.30, at least about 1:1.10:1.35, at least about 1:1.10:1.40, at least about 1:1.10:1.45, at least about 1:1.10:1.50, at least about 1:1.10:1.55, at least about 1:1.10:1.60, at least about 1:1.10:1.65, at least about 1:1.10:1.70, at least about 1:1.10:1.75, at least about 1:1.10:1.80, at least about 1:1.10:1.85, at least about 1:1.10:1.90, at least about 1:1.10:1.95, or at least about 1:1.10:2.

In a further embodiment, the ratio of PD-1 antagonist to PVRIG antagonist to CD96 antagonist may be at least about 1:1.15:0.25, at least about 1:1.15:0.30, at least about 1:1.15:0.35, at least about 1:1.15:0.40, at least about 1:1.15:0.45, at least about 1:1.15:0.50, at least about 1:1.15:0.55, at least about 1:1.15:0.60, at least about 1:1.15:0.65, at least about 1:1.15:0.70, at least about 1:1.15:0.75, at least about 1:1.15:0.80, at least about 1:1.15:0.85, at least about 1:1.15:0.90, at least about 1:1.15:0.95, at least about 1:1.15:1, at least about 1:1.15:1.05, at least about 1:1.15:1.10, at least about 1:1.15:1.15, at least about 1:1.15:1.20, at least about 1:1.15:1.25, at least about 1:1.15:1.30, at least about 1:1.15:1.35, at least about 1:1.15:1.40, at least about 1:1.15:1.45, at least about 1:1.15:1.50, at least about 1:1.15:1.55, at least about 1:1.15:1.60, at least about 1:1.15:1.65, at least about 1:1.15:1.70, at least about 1:1.15:1.75, at least about 1:1.15:1.80, at least about 1:1.15:1.85, at least about 1:1.15:1.90, at least about 1:1.15:1.95, or at least about 1:1.15:2.

In a further embodiment, the ratio of PD-1 antagonist to PVRIG antagonist to CD96 antagonist may be at least about 1:1.20:0.25, at least about 1:1.20:0.30, at least about 1:1.20:0.35, at least about 1:1.20:0.40, at least about 1:1.20:0.45, at least about 1:1.20:0.50, at least about 1:1.20:0.55, at least about 1:1.20:0.60, at least about 1:1.20:0.65, at least about 1:1.20:0.70, at least about 1:1.20:0.75, at least about 1:1.20:0.80, at least about 1:1.20:0.85, at least about 1:1.20:0.90, at least about 1:1.20:0.95, at least about 1:1.20:1, at least about 1:1.20:1.05, at least about 1:1.20:1.10, at least about 1:1.20:1.15, at least about 1:1.20:1.20, at least about 1:1.20:1.25, at least about 1:1.20:1.30, at least about 1:1.20:1.35, at least about 1:1.20:1.40, at least about 1:1.20:1.45, at least about 1:1.20:1.50, at least about 1:1.20:1.55, at least about 1:1.20:1.60, at least about 1:1.20:1.65, at least about 1:1.20:1.70, at least about 1:1.20:1.75, at least about 1:1.20:1.80, at least about 1:1.20:1.85, at least about 1:1.20:1.90, at least about 1:1.20:1.95, or at least about 1:1.20:2.

In a further embodiment, the ratio of PD-1 antagonist to PVRIG antagonist to CD96 antagonist may be at least about 1:1.25:0.25, at least about 1:1.25:0.30, at least about 1:1.25:0.35, at least about 1:1.25:0.40, at least about 1:1.25:0.45, at least about 1:1.25:0.50, at least about 1:1.25:0.55, at least about 1:1.25:0.60, at least about 1:1.25:0.65, at least about 1:1.25:0.70, at least about 1:1.25:0.75, at least about 1:1.25:0.80, at least about 1:1.25:0.85, at least about 1:1.25:0.90, at least about 1:1.25:0.95, at least about 1:1.25:1, at least about 1:1.25:1.05, at least about 1:1.25:1.10, at least about 1:1.25:1.15, at least about 1:1.25:1.20, at least about 1:1.25:1.25, at least about 1:1.25:1.30, at least about 1:1.25:1.35, at least about 1:1.25:1.40, at least about 1:1.25:1.45, at least about 1:1.25:1.50, at least about 1:1.25:1.55, at least about 1:1.25:1.60, at least about 1:1.25:1.65, at least about 1:1.25:1.70, at least about 1:1.25:1.75, at least about 1:1.25:1.80, at least about 1:1.25:1.85, at least about 1:1.25:1.90, at least about 1:1.25:1.95, or at least about 1:1.25:2.

In a further embodiment, the ratio of PD-1 antagonist to PVRIG antagonist to CD96 antagonist may be at least about 1:1.30:0.25, at least about 1:1.30:0.30, at least about 1:1.30:0.35, at least about 1:1.30:0.40, at least about 1:1.30:0.45, at least about 1:1.30:0.50, at least about 1:1.30:0.55, at least about 1:1.30:0.60, at least about 1:1.30:0.65, at least about 1:1.30:0.70, at least about 1:1.30:0.75, at least about 1:1.30:0.80, at least about 1:1.30:0.85, at least about 1:1.30:0.90, at least about 1:1.30:0.95, at least about 1:1.30:1, at least about 1:1.30:1.05, at least about 1:1.30:1.10, at least about 1:1.30:1.15, at least about 1:1.30:1.20, at least about 1:1.30:1.25, at least about 1:1.30:1.30, at least about 1:1.30:1.35, at least about 1:1.30:1.40, at least about 1:1.30:1.45, at least about 1:1.30:1.50, at least about 1:1.30:1.55, at least about 1:1.30:1.60, at least about 1:1.30:1.65, at least about 1:1.30:1.70, at least about 1:1.30:1.75, at least about 1:1.30:1.80, at least about 1:1.30:1.85, at least about 1:1.30:1.90, at least about 1:1.30:1.95, or at least about 1:1.30:2.

In a further embodiment, the ratio of PD-1 antagonist to PVRIG antagonist to CD96 antagonist may be at least about 1:1.35:0.25, at least about 1:1.35:0.30, at least about 1:1.35:0.35, at least about 1:1.35:0.40, at least about 1:1.35:0.45, at least about 1:1.35:0.50, at least about 1:1.35:0.55, at least about 1:1.35:0.65, at least about 1:1.35:0.70, at least about 1:1.35:0.75, at least about 1:1.35:0.80, at least about 1:1.35:0.85, at least about 1:1.35:0.90, at least about 1:1.35:0.95, at least about 1:1.35:1, at least about 1:1.35:1.05, at least about 1:1.35:1.10, at least about 1:1.35:1.15, at least about 1:1.35:1.20, at least about 1:1.35:1.25, at least about 1:1.35:1.30, at least about 1:1.35:1.35, at least about 1:1.35:1.40, at least about 1:1.35:1.45, at least about 1:1.35:1.50, at least about 1:1.35:1.55, at least about 1:1.35:1.60, at least about 1:1.35:1.65, at least about 1:1.35:1.70, at least about 1:1.35:1.75, at least about 1:1.35:1.80, at least about 1:1.35:1.85, at least about 1:1.35:1.90, at least about 1:1.35:1.95, or at least about 1:1.35:2.

In a further embodiment, the ratio of PD-1 antagonist to PVRIG antagonist to CD96 antagonist may be at least about 1:1.40:0.25, at least about 1:1.40:0.30, at least about 1:1.40:0.35, at least about 1:1.40:0.40, at least about 1:1.40:0.45, at least about 1:1.40:0.50, at least about 1:1.40:0.55, at least about 1:1.40:0.60, at least about 1:1.40:0.65, at least about 1:1.40:0.70, at least about 1:1.40:0.75, at least about 1:1.40:0.80, at least about 1:1.40:0.85, at least about 1:1.40:0.90, at least about 1:1.40:0.95, at least about 1:1.40:1, at least about 1:1.40:1.05, at least about 1:1.40:1.10, at least about 1:1.40:1.15, at least about 1:1.40:1.20, at least about 1:1.40:1.25, at least about 1:1.40:1.30, at least about 1:1.40:1.35, at least about 1:1.40:1.40, at least about 1:1.40:1.45, at least about 1:1.40:1.50, at least about 1:1.40:1.55, at least about 1:1.40:1.60, at least about 1:1.40:1.65, at least about 1:1.40:1.70, at least about 1:1.40:1.75, at least about 1:1.40:1.80, at least about 1:1.40:1.85, at least about 1:1.40:1.90, at least about 1:1.40:1.95, or at least about 1:1.40:2.

In a further embodiment, the ratio of PD-1 antagonist to PVRIG antagonist to CD96 antagonist may be at least about 1:1.45:0.25, at least about 1:1.45:0.30, at least about 1:1.45:0.35, at least about 1:1.45:0.40, at least about 1:1.45:0.45, at least about 1:1.45:0.50, at least about 1:1.45:0.55, at least about 1:1.45:0.60, at least about 1:1.45:0.65, at least about 1:1.45:0.70, at least about 1:1.45:0.75, at least about 1:1.45:0.80, at least about 1:1.45:0.85, at least about 1:1.45:0.90, at least about 1:1.45:0.95, at least about 1:1.45:1, at least about 1:1.45:1.05, at least about 1:1.45:1.10, at least about 1:1.45:1.15, at least about 1:1.45:1.20, at least about 1:1.45:1.25, at least about 1:1.45:1.30, at least about 1:1.45:1.35, at least about 1:1.45:1.40, at least about 1:1.45:1.45, at least about 1:1.45:1.50, at least about 1:1.45:1.55, at least about 1:1.45:1.60, at least about 1:1.45:1.65, at least about 1:1.45:1.70, at least about 1:1.45:1.75, at least about 1:1.45:1.80, at least about 1:1.45:1.85, at least about 1:1.45:1.90, at least about 1:1.45:1.95, or at least about 1:1.45:2.

In a further embodiment, the ratio of PD-1 antagonist to PVRIG antagonist to CD96 antagonist may be at least about 1:1.50:0.25, at least about 1:1.50:0.30, at least about 1:1.50:0.35, at least about 1:1.50:0.40, at least about 1:1.50:0.45, at least about 1:1.50:0.50, at least about 1:1.50:0.55, at least about 1:1.50:0.60, at least about 1:1.50:0.65, at least about 1:1.50:0.70, at least about 1:1.50:0.75, at least about 1:1.50:0.80, at least about 1:1.50:0.85, at least about 1:1.50:0.90, at least about 1:1.50:0.95, at least about 1:1.50:1, at least about 1:1.50:1.05, at least about 1:1.50:1.10, at least about 1:1.50:1.15, at least about 1:1.50:1.20, at least about 1:1.50:1.25, at least about 1:1.50:1.30, at least about 1:1.50:1.35, at least about 1:1.50:1.40, at least about 1:1.50:1.45, at least about 1:1.50:1.50, at least about 1:1.50:1.55, at least about 1:1.50:1.60, at least about 1:1.50:1.65, at least about 1:1.50:1.70, at least about 1:1.50:1.75, at least about 1:1.50:1.80, at least about 1:1.50:1.85, at least about 1:1.50:1.90, at least about 1:1.50:1.95, or at least about 1:1.50:2.

In a further embodiment, the ratio of PD-1 antagonist to PVRIG antagonist to CD96 antagonist may be at least about 1:1.55:0.25, at least about 1:1.55:0.30, at least about 1:1.55:0.35, at least about 1:1.55:0.40, at least about 1:1.55:0.45, at least about 1:1.55:0.50, at least about 1:1.55:0.55, at least about 1:1.55:0.60, at least about 1:1.55:0.65, at least about 1:1.55:0.70, at least about 1:1.55:0.75, at least about 1:1.55:0.80, at least about 1:1.55:0.85, at least about 1:1.55:0.90, at least about 1:1.55:0.95, at least about 1:1.55:1, at least about 1:1.55:1.05, at least about 1:1.55:1.10, at least about 1:1.55:1.15, at least about 1:1.55:1.20, at least about 1:1.55:1.25, at least about 1:1.55:1.30, at least about 1:1.55:1.35, at least about 1:1.55:1.40, at least about 1:1.55:1.45, at least about 1:1.55:1.50, at least about 1:1.55:1.55, at least about 1:1.55:1.60, at least about 1:1.55:1.65, at least about 1:1.55:1.70, at least about 1:1.55:1.75, at least about 1:1.55:1.80, at least about 1:1.55:1.85, at least about 1:1.55:1.90, at least about 1:1.55:1.95, or at least about 1:1.55:2.

In a further embodiment, the ratio of PD-1 antagonist to PVRIG antagonist to CD96 antagonist may be at least about 1:1.60:0.25, at least about 1:1.60:0.30, at least about 1:1.60:0.35, at least about 1:1.60:0.40, at least about 1:1.60:0.45, at least about 1:1.60:0.50, at least about 1:1.60:0.55, at least about 1:1.60:0.60, at least about 1:1.60:0.65, at least about 1:1.60:0.70, at least about 1:1.60:0.75, at least about 1:1.60:0.80, at least about 1:1.60:0.85, at least about 1:1.60:0.90, at least about 1:1.60:0.95, at least about 1:1.60:1, at least about 1:1.60:1.05, at least about 1:1.60:1.10, at least about 1:1.60:1.15, at least about 1:1.60:1.20, at least about 1:1.60:1.25, at least about 1:1.60:1.30, at least about 1:1.60:1.35, at least about 1:1.60:1.40, at least about 1:1.60:1.45, at least about 1:1.60:1.50, at least about 1:1.60:1.55, at least about 1:1.60:1.60, at least about 1:1.60:1.65, at least about 1:1.60:1.70, at least about 1:1.60:1.75, at least about 1:1.60:1.80, at least about 1:1.60:1.85, at least about 1:1.60:1.90, at least about 1:1.60:1.95, or at least about 1:1.60:2.

In a further embodiment, the ratio of PD-1 antagonist to PVRIG antagonist to CD96 antagonist may be at least about 1:1.65:0.25, at least about 1:1.65:0.30, at least about 1:1.65:0.35, at least about 1:1.65:0.40, at least about 1:1.65:0.45, at least about 1:1.65:0.50, at least about 1:1.65:0.55, at least about 1:1.65:0.60, at least about 1:1.65:0.65, at least about 1:1.65:0.70, at least about 1:1.65:0.75, at least about 1:1.65:0.80, at least about 1:1.65:0.85, at least about 1:1.65:0.90, at least about 1:1.65:0.95, at least about 1:1.65:1, at least about 1:1.65:1.05, at least about 1:1.65:1.10, at least about 1:1.65:1.15, at least about 1:1.65:1.20, at least about 1:1.65:1.25, at least about 1:1.65:1.30, at least about 1:1.65:1.35, at least about 1:1.65:1.40, at least about 1:1.65:1.45, at least about 1:1.65:1.50, at least about 1:1.65:1.55, at least about 1:1.65:1.60, at least about 1:1.65:1.65, at least about 1:1.65:1.70, at least about 1:1.65:1.75, at least about 1:1.65:1.80, at least about 1:1.65:1.85, at least about 1:1.65:1.90, at least about 1:1.65:1.95, or at least about 1:1.65:2.

In a further embodiment, the ratio of PD-1 antagonist to PVRIG antagonist to CD96 antagonist may be at least about 1:1.70:0.25, at least about 1:1.70:0.30, at least about 1:1.70:0.35, at least about 1:1.70:0.40, at least about 1:1.70:0.45, at least about 1:1.70:0.50, at least about 1:1.70:0.55, at least about 1:1.70:0.60, at least about 1:1.70:0.65, at least about 1:1.70:0.70, at least about 1:1.70:0.75, at least about 1:1.70:0.80, at least about 1:1.70:0.85, at least about 1:1.70:0.90, at least about 1:1.70:0.95, at least about 1:1.70:1, at least about 1:1.70:1.05, at least about 1:1.70:1.10, at least about 1:1.70:1.15, at least about 1:1.70:1.20, at least about 1:1.70:1.25, at least about 1:1.70:1.30, at least about 1:1.70:1.35, at least about 1:1.70:1.40, at least about 1:1.70:1.45, at least about 1:1.70:1.50, at least about 1:1.70:1.55, at least about 1:1.70:1.60, at least about 1:1.70:1.65, at least about 1:1.70:1.70, at least about 1:1.70:1.75, at least about 1:1.70:1.80, at least about 1:1.70:1.85, at least about 1:1.70:1.90, at least about 1:1.70:1.95, or at least about 1:1.70:2.

In a further embodiment, the ratio of PD-1 antagonist to PVRIG antagonist to CD96 antagonist may be at least about 1:1.75:0.25, at least about 1:1.75:0.30, at least about 1:1.75:0.35, at least about 1:1.75:0.40, at least about 1:1.75:0.45, at least about 1:1.75:0.50, at least about 1:1.75:0.55, at least about 1:1.75:0.60, at least about 1:1.75:0.65, at least about 1:1.75:0.70, at least about 1:1.75:0.75, at least about 1:1.75:0.80, at least about 1:1.75:0.85, at least about 1:1.75:0.90, at least about 1:1.75:0.95, at least about 1:1.75:1, at least about 1:1.75:1.05, at least about 1:1.75:1.10, at least about 1:1.75:1.15, at least about 1:1.75:1.20, at least about 1:1.75:1.25, at least about 1:1.75:1.30, at least about 1:1.75:1.35, at least about 1:1.75:1.40, at least about 1:1.75:1.45, at least about 1:1.75:1.50, at least about 1:1.75:1.55, at least about 1:1.75:1.60, at least about 1:1.75:1.65, at least about 1:1.75:1.70, at least about 1:1.75:1.75, at least about 1:1.75:1.80, at least about 1:1.75:1.85, at least about 1:1.75:1.90, at least about 1:1.75:1.95, or at least about 1:1.75:2.

In a further embodiment, the ratio of PD-1 antagonist to PVRIG antagonist to CD96 antagonist may be at least about 1:1.80:0.25, at least about 1:1.80:0.30, at least about 1:1.80:0.35, at least about 1:1.80:0.40, at least about 1:1.80:0.45, at least about 1:1.80:0.50, at least about 1:1.80:0.55, at least about 1:1.80:0.60, at least about 1:1.80:0.65, at least about 1:1.80:0.70, at least about 1:1.80:0.75, at least about 1:1.80:0.80, at least about 1:1.80:0.85, at least about 1:1.80:0.90, at least about 1:1.80:0.95, at least about 1:1.80:1, at least about 1:1.80:1.05, at least about 1:1.80:1.10, at least about 1:1.80:1.15, at least about 1:1.80:1.20, at least about 1:1.80:1.25, at least about 1:1.80:1.30, at least about 1:1.80:1.35, at least about 1:1.80:1.40, at least about 1:1.80:1.45, at least about 1:1.80:1.50, at least about 1:1.80:1.55, at least about 1:1.80:1.60, at least about 1:1.80:1.65, at least about 1:1.80:1.70, at least about 1:1.80:1.75, at least about 1:1.80:1.80, at least about 1:1.80:1.85, at least about 1:1.80:1.90, at least about 1:1.80:1.95, or at least about 1:1.80:2.

In a further embodiment, the ratio of PD-1 antagonist to PVRIG antagonist to CD96 antagonist may be at least about 1:1.85:0.25, at least about 1:1.85:0.30, at least about 1:1.85:0.35, at least about 1:1.85:0.40, at least about 1:1.85:0.45, at least about 1:1.85:0.50, at least about 1:1.85:0.55, at least about 1:1.85:0.60, at least about 1:1.85:0.65, at least about 1:1.85:0.70, at least about 1:1.85:0.75, at least about 1:1.85:0.80, at least about 1:1.85:0.85, at least about 1:1.85:0.90, at least about 1:1.85:0.95, at least about 1:1.85:1, at least about 1:1.85:1.05, at least about 1:1.85:1.10, at least about 1:1.85:1.15, at least about 1:1.85:1.20, at least about 1:1.85:1.25, at least about 1:1.85:1.30, at least about 1:1.85:1.35, at least about 1:1.85:1.40, at least about 1:1.85:1.45, at least about 1:1.85:1.50, at least about 1:1.85:1.55, at least about 1:1.85:1.60, at least about 1:1.85:1.65, at least about 1:1.85:1.70, at least about 1:1.85:1.75, at least about 1:1.85:1.80, at least about 1:1.85:1.85, at least about 1:1.85:1.90, at least about 1:1.85:1.95, or at least about 1:1.85:2.

In a further embodiment, the ratio of PD-1 antagonist to PVRIG antagonist to CD96 antagonist may be at least about 1:1.90:0.25, at least about 1:1.90:0.30, at least about 1:1.90:0.35, at least about 1:1.90:0.40, at least about 1:1.90:0.45, at least about 1:1.90:0.50, at least about 1:1.90:0.55, at least about 1:1.90:0.60, at least about 1:1.90:0.65, at least about 1:1.90:0.70, at least about 1:1.90:0.75, at least about 1:1.90:0.80, at least about 1:1.90:0.85, at least about 1:1.90:0.90, at least about 1:1.90:0.95, at least about 1:1.90:1, at least about 1:1.90:1.05, at least about 1:1.90:1.10, at least about 1:1.90:1.15, at least about 1:1.90:1.20, at least about 1:1.90:1.25, at least about 1:1.90:1.30, at least about 1:1.90:1.35, at least about 1:1.90:1.40, at least about 1:1.90:1.45, at least about 1:1.90:1.50, at least about 1:1.90:1.55, at least about 1:1.90:1.60, at least about 1:1.90:1.65, at least about 1:1.90:1.70, at least about 1:1.90:1.75, at least about 1:1.90:1.80, at least about 1:1.90:1.85, at least about 1:1.90:1.90, at least about 1:1.90:1.95, or at least about 1:1.90:2.

In a further embodiment, the ratio of PD-1 antagonist to PVRIG antagonist to CD96 antagonist may be at least about 1:1.95:0.25, at least about 1:1.95:0.30, at least about 1:1.95:0.35, at least about 1:1.95:0.40, at least about 1:1.95:0.45, at least about 1:1.95:0.50, at least about 1:1.95:0.55, at least about 1:1.95:0.60, at least about 1:1.95:0.65, at least about 1:1.95:0.70, at least about 1:1.95:0.75, at least about 1:1.95:0.80, at least about 1:1.95:0.85, at least about 1:1.95:0.90, at least about 1:1.95:0.95, at least about 1:1.95:1, at least about 1:1.95:1.05, at least about 1:1.95:1.10, at least about 1:1.95:1.15, at least about 1:1.95:1.20, at least about 1:1.95:1.25, at least about 1:1.95:1.30, at least about 1:1.95:1.35, at least about 1:1.95:1.40, at least about 1:1.95:1.45, at least about 1:1.95:1.50, at least about 1:1.95:1.55, at least about 1:1.95:1.60, at least about 1:1.95:1.65, at least about 1:1.95:1.70, at least about 1:1.95:1.75, at least about 1:1.95:1.80, at least about 1:1.95:1.85, at least about 1:1.95:1.90, at least about 1:1.95:1.95, or at least about 1:1.95:2.

In a further embodiment, the ratio of PD-1 antagonist to PVRIG antagonist to CD96 antagonist may be at least about 1:2:0.25, at least about 1:2:0.30, at least about 1:2:0.35, at least about 1:2:0.40, at least about 1:2:0.45, at least about 1:2:0.50, at least about 1:2:0.55, at least about 1:2:0.60, at least about 1:2:0.65, at least about 1:2:0.70, at least about 1:2:0.75, at least about 1:2:0.80, at least about 1:2:0.85, at least about 1:2:0.90, at least about 1:2:0.95, at least about 1:2:1, at least about 1:2:1.05, at least about 1:2:1.10, at least about 1:2:1.15, at least about 1:2:1.20, at least about 1:2:1.25, at least about 1:2:1.30, at least about 1:2:1.35, at least about 1:2:1.40, at least about 1:2:1.45, at least about 1:2:1.50, at least about 1:2:1.55, at least about 1:2:1.60, at least about 1:2:1.65, at least about 1:2:1.70, at least about 1:2:1.75, at least about 1:2:1.80, at least about 1:2:1.85, at least about 1:2:1.90, at least about 1:2:1.95, or at least about 1:2:2.

In a further embodiment, the ratio of PD-1 antagonist to PVRIG antagonist to CD96 antagonist may be at least about 1:0.25:0.25, at least about 1:0.30:0.25, at least about 1:0.35:0.25, at least about 1:0.40:0.25, at least about 1:0.45:0.25, at least about 1:0.50:0.25, at least about 1:0.55:0.25, at least about 1:0.60:0.25, at least about 1:0.65:0.25, at least about 1:0.70:0.25, at least about 1:0.75:0.25, at least about 1:0.80:0.25, at least about 1:0.85:0.25, at least about 1:0.90:0.25, at least about 1:0.95:0.25, at least about 1:1:0.25, at least about 1:1.05:0.25, at least about 1:1.10:0.25, at least about 1:1.15:0.25, at least about 1:1.20:0.25, at least about 1:1.25:0.25, at least about 1:1.30:0.25, at least about 1:1.35:0.25, at least about 1:1.40:0.25, at least about 1:1.45:0.25, at least about 1:1.50:0.25, at least about 1:1.55:0.25, at least about 1:1.60:0.25, at least about 1:1.65:0.25, at least about 1:1.70:0.25, at least about 1:1.75:0.25, at least about 1:1.80:0.25, at least about 1:1.85:0.25, at least about 1:1.90:0.25, at least about 1:1.95:0.25, or at least about 1:2:0.25.

In a further embodiment, the ratio of PD-1 antagonist to PVRIG antagonist to CD96 antagonist may be at least about 1:0.25:0.30, at least about 1:0.30:0.30, at least about 1:0.35:0.30, at least about 1:0.40:0.30, at least about 1:0.45:0.30, at least about 1:0.50:0.30, at least about 1:0.55:0.30, at least about 1:0.60:0.30, at least about 1:0.65:0.30, at least about 1:0.70:0.30, at least about 1:0.75:0.30, at least about 1:0.80:0.30, at least about 1:0.85:0.30, at least about 1:0.90:0.30, at least about 1:0.95:0.30, at least about 1:1:0.30, at least about 1:1.05:0.30, at least about 1:1.10:0.30, at least about 1:1.15:0.30, at least about 1:1.20:0.30, at least about 1:1.25:0.30, at least about 1:1.30:0.30, at least about 1:1.35:0.30, at least about 1:1.40:0.30, at least about 1:1.45:0.30, at least about 1:1.50:0.30, at least about 1:1.55:0.30, at least about 1:1.60:0.30, at least about 1:1.65:0.30, at least about 1:1.70:0.30, at least about 1:1.75:0.30, at least about 1:1.80:0.30, at least about 1:1.85:0.30, at least about 1:1.90:0.30, at least about 1:1.95:0.30, or at least about 1:2:0.30.

In a further embodiment, the ratio of PD-1 antagonist to PVRIG antagonist to CD96 antagonist may be at least about 1:0.25:0.35, at least about 1:0.30:0.35, at least about 1:0.35:0.35, at least about 1:0.40:0.35, at least about 1:0.45:0.35, at least about 1:0.50:0.35, at least about 1:0.55:0.35, at least about 1:0.60:0.35, at least about 1:0.65:0.35, at least about 1:0.70:0.35, at least about 1:0.75:0.35, at least about 1:0.80:0.35, at least about 1:0.85:0.35, at least about 1:0.90:0.35, at least about 1:0.95:0.35, at least about 1:1:0.35, at least about 1:1.05:0.35, at least about 1:1.10:0.35, at least about 1:1.15:0.35, at least about 1:1.20:0.35, at least about 1:1.25:0.35, at least about 1:1.30:0.35, at least about 1:1.35:0.35, at least about 1:1.40:0.35, at least about 1:1.45:0.35, at least about 1:1.50:0.35, at least about 1:1.55:0.35, at least about 1:1.60:0.35, at least about 1:1.65:0.35, at least about 1:1.70:0.35, at least about 1:1.75:0.35, at least about 1:1.80:0.35, at least about 1:1.85:0.35, at least about 1:1.90:0.35, at least about 1:1.95:0.35, or at least about 1:2:0.35.

In a further embodiment, the ratio of PD-1 antagonist to PVRIG antagonist to CD96 antagonist may be at least about 1:0.25:0.40, at least about 1:0.30:0.40, at least about 1:0.35:0.40, at least about 1:0.40:0.40, at least about 1:0.45:0.40, at least about 1:0.50:0.40, at least about 1:0.55:0.40, at least about 1:0.60:0.40, at least about 1:0.65:0.40, at least about 1:0.70:0.40, at least about 1:0.75:0.40, at least about 1:0.80:0.40, at least about 1:0.85:0.40, at least about 1:0.90:0.40, at least about 1:0.95:0.40, at least about 1:1:0.40, at least about 1:1.05:0.40, at least about 1:1.10:0.40, at least about 1:1.15:0.40, at least about 1:1.20:0.40, at least about 1:1.25:0.40, at least about 1:1.30:0.40, at least about 1:1.35:0.40, at least about 1:1.40:0.40, at least about 1:1.45:0.40, at least about 1:1.50:0.40, at least about 1:1.55:0.40, at least about 1:1.60:0.40, at least about 1:1.65:0.40, at least about 1:1.70:0.40, at least about 1:1.75:0.40, at least about 1:1.80:0.40, at least about 1:1.85:0.40, at least about 1:1.90:0.40, at least about 1:1.95:0.40, or at least about 1:2:0.40.

In a further embodiment, the ratio of PD-1 antagonist to PVRIG antagonist to CD96 antagonist may be at least about 1:0.25:0.45, at least about 1:0.30:0.45, at least about 1:0.35:0.45, at least about 1:0.40:0.45, at least about 1:0.45:0.45, at least about 1:0.50:0.45, at least about 1:0.55:0.45, at least about 1:0.60:0.45, at least about 1:0.65:0.45, at least about 1:0.70:0.45, at least about 1:0.75:0.45, at least about 1:0.80:0.45, at least about 1:0.85:0.45, at least about 1:0.90:0.45, at least about 1:0.95:0.45, at least about 1:1:0.45, at least about 1:1.05:0.45, at least about 1:1.10:0.45, at least about 1:1.15:0.45, at least about 1:1.20:0.45, at least about 1:1.25:0.45, at least about 1:1.30:0.45, at least about 1:1.35:0.45, at least about 1:1.40:0.45, at least about 1:1.45:0.45, at least about 1:1.50:0.45, at least about 1:1.55:0.45, at least about 1:1.60:0.45, at least about 1:1.65:0.45, at least about 1:1.70:0.45, at least about 1:1.75:0.45, at least about 1:1.80:0.45, at least about 1:1.85:0.45, at least about 1:1.90:0.45, at least about 1:1.95:0.45, or at least about 1:2:0.45.

In a further embodiment, the ratio of PD-1 antagonist to PVRIG antagonist to CD96 antagonist may be at least about 1:0.25:0.50, at least about 1:0.30:0.50, at least about 1:0.35:0.50, at least about 1:0.40:0.50, at least about 1:0.45:0.50, at least about 1:0.50:0.50, at least about 1:0.55:0.50, at least about 1:0.60:0.50, at least about 1:0.65:0.50, at least about 1:0.70:0.50, at least about 1:0.75:0.50, at least about 1:0.80:0.50, at least about 1:0.85:0.50, at least about 1:0.90:0.50, at least about 1:0.95:0.50, at least about 1:1:0.50, at least about 1:1.05:0.50, at least about 1:1.10:0.50, at least about 1:1.15:0.50, at least about 1:1.20:0.50, at least about 1:1.25:0.50, at least about 1:1.30:0.50, at least about 1:1.35:0.50, at least about 1:1.40:0.50, at least about 1:1.45:0.50, at least about 1:1.50:0.50, at least about 1:1.55:0.50, at least about 1:1.60:0.50, at least about 1:1.65:0.50, at least about 1:1.70:0.50, at least about 1:1.75:0.50, at least about 1:1.80:0.50, at least about 1:1.85:0.50, at least about 1:1.90:0.50, at least about 1:1.95:0.50, or at least about 1:2:0.50.

In a further embodiment, the ratio of PD-1 antagonist to PVRIG antagonist to CD96 antagonist may be at least about 1:0.25:0.55, at least about 1:0.30:0.55, at least about 1:0.35:0.55, at least about 1:0.40:0.55, at least about 1:0.45:0.55, at least about 1:0.50:0.55, at least about 1:0.55:0.55, at least about 1:0.60:0.55, at least about 1:0.65:0.55, at least about 1:0.70:0.55, at least about 1:0.75:0.55, at least about 1:0.80:0.55, at least about 1:0.85:0.55, at least about 1:0.90:0.55, at least about 1:0.95:0.55, at least about 1:1:0.55, at least about 1:1.05:0.55, at least about 1:1.10:0.55, at least about 1:1.15:0.55, at least about 1:1.20:0.55, at least about 1:1.25:0.55, at least about 1:1.30:0.55, at least about 1:1.35:0.55, at least about 1:1.40:0.55, at least about 1:1.45:0.55, at least about 1:1.50:0.55, at least about 1:1.55:0.55, at least about 1:1.60:0.55, at least about 1:1.65:0.55, at least about 1:1.70:0.55, at least about 1:1.75:0.55, at least about 1:1.80:0.55, at least about 1:1.85:0.55, at least about 1:1.90:0.55, at least about 1:1.95:0.55, or at least about 1:2:0.55.

In a further embodiment, the ratio of PD-1 antagonist to PVRIG antagonist to CD96 antagonist may be at least about 1:0.25:0.60, at least about 1:0.30:0.60, at least about 1:0.35:0.60, at least about 1:0.40:0.60, at least about 1:0.45:0.60, at least about 1:0.50:0.60, at least about 1:0.55:0.60, at least about 1:0.60:0.60, at least about 1:0.65:0.60, at least about 1:0.70:0.60, at least about 1:0.75:0.60, at least about 1:0.80:0.60, at least about 1:0.85:0.60, at least about 1:0.90:0.60, at least about 1:0.95:0.60, at least about 1:1:0.60, at least about 1:1.05:0.60, at least about 1:1.10:0.60, at least about 1:1.15:0.60, at least about 1:1.20:0.60, at least about 1:1.25:0.60, at least about 1:1.30:0.60, at least about 1:1.35:0.60, at least about 1:1.40:0.60, at least about 1:1.45:0.60, at least about 1:1.50:0.60, at least about 1:1.55:0.60, at least about 1:1.60:0.60, at least about 1:1.65:0.60, at least about 1:1.70:0.60, at least about 1:1.75:0.60, at least about 1:1.80:0.60, at least about 1:1.85:0.60, at least about 1:1.90:0.60, at least about 1:1.95:0.60, or at least about 1:2:0.60.

In a further embodiment, the ratio of PD-1 antagonist to PVRIG antagonist to CD96 antagonist may be at least about 1:0.25:0.65, at least about 1:0.30:0.65, at least about 1:0.35:0.65, at least about 1:0.40:0.65, at least about 1:0.45:0.65, at least about 1:0.50:0.65, at least about 1:0.55:0.65, at least about 1:0.60:0.65, at least about 1:0.65:0.65, at least about 1:0.70:0.65, at least about 1:0.75:0.65, at least about 1:0.80:0.65, at least about 1:0.85:0.65, at least about 1:0.90:0.65, at least about 1:0.95:0.65, at least about 1:1:0.65, at least about 1:1.05:0.65, at least about 1:1.10:0.65, at least about 1:1.15:0.65, at least about 1:1.20:0.65, at least about 1:1.25:0.65, at least about 1:1.30:0.65, at least about 1:1.35:0.65, at least about 1:1.40:0.65, at least about 1:1.45:0.65, at least about 1:1.50:0.65, at least about 1:1.55:0.65, at least about 1:1.60:0.65, at least about 1:1.65:0.65, at least about 1:1.70:0.65, at least about 1:1.75:0.65, at least about 1:1.80:0.65, at least about 1:1.85:0.65, at least about 1:1.90:0.65, at least about 1:1.95:0.65, or at least about 1:2:0.65.

In a further embodiment, the ratio of PD-1 antagonist to PVRIG antagonist to CD96 antagonist may be at least about 1:0.25:0.70, at least about 1:0.30:0.70, at least about 1:0.35:0.70, at least about 1:0.40:0.70, at least about 1:0.45:0.70, at least about 1:0.50:0.70, at least about 1:0.55:0.70, at least about 1:0.60:0.70, at least about 1:0.65:0.70, at least about 1:0.70:0.70, at least about 1:0.75:0.70, at least about 1:0.80:0.70, at least about 1:0.85:0.70, at least about 1:0.90:0.70, at least about 1:0.95:0.70, at least about 1:1:0.70, at least about 1:1.05:0.70, at least about 1:1.10:0.70, at least about 1:1.15:0.70, at least about 1:1.20:0.70, at least about 1:1.25:0.70, at least about 1:1.30:0.70, at least about 1:1.35:0.70, at least about 1:1.40:0.70, at least about 1:1.45:0.70, at least about 1:1.50:0.70, at least about 1:1.55:0.70, at least about 1:1.60:0.70, at least about 1:1.65:0.70, at least about 1:1.70:0.70, at least about 1:1.75:0.70, at least about 1:1.80:0.70, at least about 1:1.85:0.70, at least about 1:1.90:0.70, at least about 1:1.95:0.70, or at least about 1:2:0.70.

In a further embodiment, the ratio of PD-1 antagonist to PVRIG antagonist to CD96 antagonist may be at least about 1:0.25:0.75, at least about 1:0.30:0.75, at least about 1:0.35:0.75, at least about 1:0.40:0.75, at least about 1:0.45:0.75, at least about 1:0.50:0.75, at least about 1:0.55:0.75, at least about 1:0.60:0.75, at least about 1:0.65:0.75, at least about 1:0.70:0.75, at least about 1:0.75:0.75, at least about 1:0.80:0.75, at least about 1:0.85:0.75, at least about 1:0.90:0.75, at least about 1:0.95:0.75, at least about 1:1:0.75, at least about 1:1.05:0.75, at least about 1:1.10:0.75, at least about 1:1.15:0.75, at least about 1:1.20:0.75, at least about 1:1.25:0.75, at least about 1:1.30:0.75, at least about 1:1.35:0.75, at least about 1:1.40:0.75, at least about 1:1.45:0.75, at least about 1:1.50:0.75, at least about 1:1.55:0.75, at least about 1:1.60:0.75, at least about 1:1.65:0.75, at least about 1:1.70:0.75, at least about 1:1.75:0.75, at least about 1:1.80:0.75, at least about 1:1.85:0.75, at least about 1:1.90:0.75, at least about 1:1.95:0.75, or at least about 1:2:0.75.

In a further embodiment, the ratio of PD-1 antagonist to PVRIG antagonist to CD96 antagonist may be at least about 1:0.25:0.80, at least about 1:0.30:0.80, at least about 1:0.35:0.80, at least about 1:0.40:0.80, at least about 1:0.45:0.80, at least about 1:0.50:0.80, at least about 1:0.55:0.80, at least about 1:0.60:0.80, at least about 1:0.65:0.80, at least about 1:0.70:0.80, at least about 1:0.75:0.80, at least about 1:0.80:0.80, at least about 1:0.85:0.80, at least about 1:0.90:0.80, at least about 1:0.95:0.80, at least about 1:1:0.80, at least about 1:1.05:0.80, at least about 1:1.10:0.80, at least about 1:1.15:0.80, at least about 1:1.20:0.80, at least about 1:1.25:0.80, at least about 1:1.30:0.80, at least about 1:1.35:0.80, at least about 1:1.40:0.80, at least about 1:1.45:0.80, at least about 1:1.50:0.80, at least about 1:1.55:0.80, at least about 1:1.60:0.80, at least about 1:1.65:0.80, at least about 1:1.70:0.80, at least about 1:1.75:0.80, at least about 1:1.80:0.80, at least about 1:1.85:0.80, at least about 1:1.90:0.80, at least about 1:1.95:0.80, or at least about 1:2:0.80.

In a further embodiment, the ratio of PD-1 antagonist to PVRIG antagonist to CD96 antagonist may be at least about 1:0.25:0.85, at least about 1:0.30:0.85, at least about 1:0.35:0.85, at least about 1:0.40:0.85, at least about 1:0.45:0.85, at least about 1:0.50:0.85, at least about 1:0.55:0.85, at least about 1:0.60:0.85, at least about 1:0.65:0.85, at least about 1:0.70:0.85, at least about 1:0.75:0.85, at least about 1:0.80:0.85, at least about 1:0.85:0.85, at least about 1:0.90:0.85, at least about 1:0.95:0.85, at least about 1:1:0.85, at least about 1:1.05:0.85, at least about 1:1.10:0.85, at least about 1:1.15:0.85, at least about 1:1.20:0.85, at least about 1:1.25:0.85, at least about 1:1.30:0.85, at least about 1:1.35:0.85, at least about 1:1.40:0.85, at least about 1:1.45:0.85, at least about 1:1.50:0.85, at least about 1:1.55:0.85, at least about 1:1.60:0.85, at least about 1:1.65:0.85, at least about 1:1.70:0.85, at least about 1:1.75:0.85, at least about 1:1.80:0.85, at least about 1:1.85:0.85, at least about 1:1.90:0.85, at least about 1:1.95:0.85, or at least about 1:2:0.85.

In a further embodiment, the ratio of PD-1 antagonist to PVRIG antagonist to CD96 antagonist may be at least about 1:0.25:0.90, at least about 1:0.30:0.90, at least about 1:0.35:0.90, at least about 1:0.40:0.90, at least about 1:0.45:0.90, at least about 1:0.50:0.90, at least about 1:0.55:0.90, at least about 1:0.60:0.90, at least about 1:0.65:0.90, at least about 1:0.70:0.90, at least about 1:0.75:0.90, at least about 1:0.80:0.90, at least about 1:0.85:0.90, at least about 1:0.90:0.90, at least about 1:0.95:0.90, at least about 1:1:0.90, at least about 1:1.05:0.90, at least about 1:1.10:0.90, at least about 1:1.15:0.90, at least about 1:1.20:0.90, at least about 1:1.25:0.90, at least about 1:1.30:0.90, at least about 1:1.35:0.90, at least about 1:1.40:0.90, at least about 1:1.45:0.90, at least about 1:1.50:0.90, at least about 1:1.55:0.90, at least about 1:1.60:0.90, at least about 1:1.65:0.90, at least about 1:1.70:0.90, at least about 1:1.75:0.90, at least about 1:1.80:0.90, at least about 1:1.85:0.90, at least about 1:1.90:0.90, at least about 1:1.95:0.90, or at least about 1:2:0.90.

In a further embodiment, the ratio of PD-1 antagonist to PVRIG antagonist to CD96 antagonist may be at least about 1:0.25:0.95, at least about 1:0.30:0.95, at least about 1:0.35:0.95, at least about 1:0.40:0.95, at least about 1:0.45:0.95, at least about 1:0.50:0.95, at least about 1:0.55:0.95, at least about 1:0.60:0.95, at least about 1:0.65:0.95, at least about 1:0.70:0.95, at least about 1:0.75:0.95, at least about 1:0.80:0.95, at least about 1:0.85:0.95, at least about 1:0.90:0.95, at least about 1:0.95:0.95, at least about 1:1:0.95, at least about 1:1.05:0.95, at least about 1:1.10:0.95, at least about 1:1.15:0.95, at least about 1:1.20:0.95, at least about 1:1.25:0.95, at least about 1:1.30:0.95, at least about 1:1.35:0.95, at least about 1:1.40:0.95, at least about 1:1.45:0.95, at least about 1:1.50:0.95, at least about 1:1.55:0.95, at least about 1:1.60:0.95, at least about 1:1.65:0.95, at least about 1:1.70:0.95, at least about 1:1.75:0.95, at least about 1:1.80:0.95, at least about 1:1.85:0.95, at least about 1:1.90:0.95, at least about 1:1.95:0.95, or at least about 1:2:0.95.

In a further embodiment, the ratio of PD-1 antagonist to PVRIG antagonist to CD96 antagonist may be at least about 1:0.25:1, at least about 1:0.30:1, at least about 1:0.35:1, at least about 1:0.40:1, at least about 1:0.45:1, at least about 1:0.50:1, at least about 1:0.55:1, at least about 1:0.60:1, at least about 1:0.65:1, at least about 1:0.70:1, at least about 1:0.75:1, at least about 1:0.80:1, at least about 1:0.85:1, at least about 1:0.90:1, at least about 1:0.95:1, at least about 1:1:1, at least about 1:1.05:1, at least about 1:1.10:1, at least about 1:1.15:1, at least about 1:1.20:1, at least about 1:1.25:1, at least about 1:1.30:1, at least about 1:1.35:1, at least about 1:1.40:1, at least about 1:1.45:1, at least about 1:1.50:1, at least about 1:1.55:1, at least about 1:1.60:1, at least about 1:1.65:1, at least about 1:1.70:1, at least about 1:1.75:1, at least about 1:1.80:1, at least about 1:1.85:1, at least about 1:1.90:1, at least about 1:1.95:1, or at least about 1:2:1.

In a further embodiment, the ratio of PD-1 antagonist to PVRIG antagonist to CD96 antagonist may be at least about 1:0.25:1.05, at least about 1:0.30:1.05, at least about 1:0.35:1.05, at least about 1:0.40:1.05, at least about 1:0.45:1.05, at least about 1:0.50:1.05, at least about 1:0.55:1.05, at least about 1:0.60:1.05, at least about 1:0.65:1.05, at least about 1:0.70:1.05, at least about 1:0.75:1.05, at least about 1:0.80:1.05, at least about 1:0.85:1.05, at least about 1:0.90:1.05, at least about 1:0.95:1.05, at least about 1:1:1.05, at least about 1:1.05:1.05, at least about 1:1.10:1.05, at least about 1:1.15:1.05, at least about 1:1.20:1.05, at least about 1:1.25:1.05, at least about 1:1.30:1.05, at least about 1:1.35:1.05, at least about 1:1.40:1.05, at least about 1:1.45:1.05, at least about 1:1.50:1.05, at least about 1:1.55:1.05, at least about 1:1.60:1.05, at least about 1:1.65:1.05, at least about 1:1.70:1.05, at least about 1:1.75:1.05, at least about 1:1.80:1.05, at least about 1:1.85:1.05, at least about 1:1.90:1.05, at least about 1:1.95:1.05, or at least about 1:2:1.05.

In a further embodiment, the ratio of PD-1 antagonist to PVRIG antagonist to CD96 antagonist may be at least about 1:0.25:1.10, at least about 1:0.30:1.10, at least about 1:0.35:1.10, at least about 1:0.40:1.10, at least about 1:0.45:1.10, at least about 1:0.50:1.10, at least about 1:0.55:1.10, at least about 1:0.60:1.10, at least about 1:0.65:1.10, at least about 1:0.70:1.10, at least about 1:0.75:1.10, at least about 1:0.80:1.10, at least about 1:0.85:1.10, at least about 1:0.90:1.10, at least about 1:0.95:1.10, at least about 1:1:1.10, at least about 1:1.05:1.10, at least about 1:1.10:1.10, at least about 1:1.15:1.10, at least about 1:1.20:1.10, at least about 1:1.25:1.10, at least about 1:1.30:1.10, at least about 1:1.35:1.10, at least about 1:1.40:1.10, at least about 1:1.45:1.10, at least about 1:1.50:1.10, at least about 1:1.55:1.10, at least about 1:1.60:1.10, at least about 1:1.65:1.10, at least about 1:1.70:1.10, at least about 1:1.75:1.10, at least about 1:1.80:1.10, at least about 1:1.85:1.10, at least about 1:1.90:1.10, at least about 1:1.95:1.10, or at least about 1:2:1.10.

In a further embodiment, the ratio of PD-1 antagonist to PVRIG antagonist to CD96 antagonist may be at least about 1:0.25:1.15, at least about 1:0.30:1.15, at least about 1:0.35:1.15, at least about 1:0.40:1.15, at least about 1:0.45:1.15, at least about 1:0.50:1.15, at least about 1:0.55:1.15, at least about 1:0.60:1.15, at least about 1:0.65:1.15, at least about 1:0.70:1.15, at least about 1:0.75:1.15, at least about 1:0.80:1.15, at least about 1:0.85:1.15, at least about 1:0.90:1.15, at least about 1:0.95:1.15, at least about 1:1:1.15, at least about 1:1.05:1.15, at least about 1:1.10:1.15, at least about 1:1.15:1.15, at least about 1:1.20:1.15, at least about 1:1.25:1.15, at least about 1:1.30:1.15, at least about 1:1.35:1.15, at least about 1:1.40:1.15, at least about 1:1.45:1.15, at least about 1:1.50:1.15, at least about 1:1.55:1.15, at least about 1:1.60:1.15, at least about 1:1.65:1.15, at least about 1:1.70:1.15, at least about 1:1.75:1.15, at least about 1:1.80:1.15, at least about 1:1.85:1.15, at least about 1:1.90:1.15, at least about 1:1.95:1.15, or at least about 1:2:1.15.

In a further embodiment, the ratio of PD-1 antagonist to PVRIG antagonist to CD96 antagonist may be at least about 1:0.25:1.20, at least about 1:0.30:1.20, at least about 1:0.35:1.20, at least about 1:0.40:1.20, at least about 1:0.45:1.20, at least about 1:0.50:1.20, at least about 1:0.55:1.20, at least about 1:0.60:1.20, at least about 1:0.65:1.20, at least about 1:0.70:1.20, at least about 1:0.75:1.20, at least about 1:0.80:1.20, at least about 1:0.85:1.20, at least about 1:0.90:1.20, at least about 1:0.95:1.20, at least about 1:1:1.20, at least about 1:1.05:1.20, at least about 1:1.10:1.20, at least about 1:1.15:1.20, at least about 1:1.20:1.20, at least about 1:1.25:1.20, at least about 1:1.30:1.20, at least about 1:1.35:1.20, at least about 1:1.40:1.20, at least about 1:1.45:1.20, at least about 1:1.50:1.20, at least about 1:1.55:1.20, at least about 1:1.60:1.20, at least about 1:1.65:1.20, at least about 1:1.70:1.20, at least about 1:1.75:1.20, at least about 1:1.80:1.20, at least about 1:1.85:1.20, at least about 1:1.90:1.20, at least about 1:1.95:1.20, or at least about 1:2:1.20.

In a further embodiment, the ratio of PD-1 antagonist to PVRIG antagonist to CD96 antagonist may be at least about 1:0.25:1.25, at least about 1:0.30:1.25, at least about 1:0.35:1.25, at least about 1:0.40:1.25, at least about 1:0.45:1.25, at least about 1:0.50:1.25, at least about 1:0.55:1.25, at least about 1:0.60:1.25, at least about 1:0.65:1.25, at least about 1:0.70:1.25, at least about 1:0.75:1.25, at least about 1:0.80:1.25, at least about 1:0.85:1.25, at least about 1:0.90:1.25, at least about 1:0.95:1.25, at least about 1:1:1.25, at least about 1:1.05:1.25, at least about 1:1.10:1.25, at least about 1:1.15:1.25, at least about 1:1.20:1.25, at least about 1:1.25:1.25, at least about 1:1.30:1.25, at least about 1:1.35:1.25, at least about 1:1.40:1.25, at least about 1:1.45:1.25, at least about 1:1.50:1.25, at least about 1:1.55:1.25, at least about 1:1.60:1.25, at least about 1:1.65:1.25, at least about 1:1.70:1.25, at least about 1:1.75:1.25, at least about 1:1.80:1.25, at least about 1:1.85:1.25, at least about 1:1.90:1.25, at least about 1:1.95:1.25, or at least about 1:2:1.25.

In a further embodiment, the ratio of PD-1 antagonist to PVRIG antagonist to CD96 antagonist may be at least about 1:0.25:1.30, at least about 1:0.30:1.30, at least about 1:0.35:1.30, at least about 1:0.40:1.30, at least about 1:0.45:1.30, at least about 1:0.50:1.30, at least about 1:0.55:1.30, at least about 1:0.60:1.30, at least about 1:0.65:1.30, at least about 1:0.70:1.30, at least about 1:0.75:1.30, at least about 1:0.80:1.30, at least about 1:0.85:1.30, at least about 1:0.90:1.30, at least about 1:0.95:1.30, at least about 1:1:1.30, at least about 1:1.05:1.30, at least about 1:1.10:1.30, at least about 1:1.15:1.30, at least about 1:1.20:1.30, at least about 1:1.25:1.30, at least about 1:1.30:1.30, at least about 1:1.35:1.30, at least about 1:1.40:1.30, at least about 1:1.45:1.30, at least about 1:1.50:1.30, at least about 1:1.55:1.30, at least about 1:1.60:1.30, at least about 1:1.65:1.30, at least about 1:1.70:1.30, at least about 1:1.75:1.30, at least about 1:1.80:1.30, at least about 1:1.85:1.30, at least about 1:1.90:1.30, at least about 1:1.95:1.30, or at least about 1:2:1.30.

In a further embodiment, the ratio of PD-1 antagonist to PVRIG antagonist to CD96 antagonist may be at least about 1:0.25:1.35, at least about 1:0.30:1.35, at least about 1:0.35:1.35, at least about 1:0.40:1.35, at least about 1:0.45:1.35, at least about 1:0.50:1.35, at least about 1:0.55:1.35, at least about 1:0.60:1.35, at least about 1:0.65:1.35, at least about 1:0.70:1.35, at least about 1:0.75:1.35, at least about 1:0.80:1.35, at least about 1:0.85:1.35, at least about 1:0.90:1.35, at least about 1:0.95:1.35, at least about 1:1:1.35, at least about 1:1.05:1.35, at least about 1:1.10:1.35, at least about 1:1.15:1.35, at least about 1:1.20:1.35, at least about 1:1.25:1.35, at least about 1:1.30:1.35, at least about 1:1.35:1.35, at least about 1:1.40:1.35, at least about 1:1.45:1.35, at least about 1:1.50:1.35, at least about 1:1.55:1.35, at least about 1:1.60:1.35, at least about 1:1.65:1.35, at least about 1:1.70:1.35, at least about 1:1.75:1.35, at least about 1:1.80:1.35, at least about 1:1.85:1.35, at least about 1:1.90:1.35, at least about 1:1.95:1.35, or at least about 1:2:1.35.

In a further embodiment, the ratio of PD-1 antagonist to PVRIG antagonist to CD96 antagonist may be at least about 1:0.25:1.40, at least about 1:0.30:1.40, at least about 1:0.35:1.40, at least about 1:0.40:1.40, at least about 1:0.45:1.40, at least about 1:0.50:1.40, at least about 1:0.55:1.40, at least about 1:0.60:1.40, at least about 1:0.65:1.40, at least about 1:0.70:1.40, at least about 1:0.75:1.40, at least about 1:0.80:1.40, at least about 1:0.85:1.40, at least about 1:0.90:1.40, at least about 1:0.95:1.40, at least about 1:1:1.40, at least about 1:1.05:1.40, at least about 1:1.10:1.40, at least about 1:1.15:1.40, at least about 1:1.20:1.40, at least about 1:1.25:1.40, at least about 1:1.30:1.40, at least about 1:1.35:1.40, at least about 1:1.40:1.40, at least about 1:1.45:1.40, at least about 1:1.50:1.40, at least about 1:1.55:1.40, at least about 1:1.60:1.40, at least about 1:1.65:1.40, at least about 1:1.70:1.40, at least about 1:1.75:1.40, at least about 1:1.80:1.40, at least about 1:1.85:1.40, at least about 1:1.90:1.40, at least about 1:1.95:1.40, or at least about 1:2:1.40.

In a further embodiment, the ratio of PD-1 antagonist to PVRIG antagonist to CD96 antagonist may be at least about 1:0.25:1.45, at least about 1:0.30:1.45, at least about 1:0.35:1.45, at least about 1:0.40:1.45, at least about 1:0.45:1.45, at least about 1:0.50:1.45, at least about 1:0.55:1.45, at least about 1:0.60:1.45, at least about 1:0.65:1.45, at least about 1:0.70:1.45, at least about 1:0.75:1.45, at least about 1:0.80:1.45, at least about 1:0.85:1.45, at least about 1:0.90:1.45, at least about 1:0.95:1.45, at least about 1:1:1.45, at least about 1:1.05:1.45, at least about 1:1.10:1.45, at least about 1:1.15:1.45, at least about 1:1.20:1.45, at least about 1:1.25:1.45, at least about 1:1.30:1.45, at least about 1:1.35:1.45, at least about 1:1.40:1.45, at least about 1:1.45:1.45, at least about 1:1.50:1.45, at least about 1:1.55:1.45, at least about 1:1.60:1.45, at least about 1:1.65:1.45, at least about 1:1.70:1.45, at least about 1:1.75:1.45, at least about 1:1.80:1.45, at least about 1:1.85:1.45, at least about 1:1.90:1.45, at least about 1:1.95:1.45, or at least about 1:2:1.45.

In a further embodiment, the ratio of PD-1 antagonist to PVRIG antagonist to CD96 antagonist may be at least about 1:0.25:1.50, at least about 1:0.30:1.50, at least about 1:0.35:1.50, at least about 1:0.40:1.50, at least about 1:0.45:1.50, at least about 1:0.50:1.50, at least about 1:0.55:1.50, at least about 1:0.60:1.50, at least about 1:0.65:1.50, at least about 1:0.70:1.50, at least about 1:0.75:1.50, at least about 1:0.80:1.50, at least about 1:0.85:1.50, at least about 1:0.90:1.50, at least about 1:0.95:1.50, at least about 1:1:1.50, at least about 1:1.05:1.50, at least about 1:1.10:1.50, at least about 1:1.15:1.50, at least about 1:1.20:1.50, at least about 1:1.25:1.50, at least about 1:1.30:1.50, at least about 1:1.35:1.50, at least about 1:1.40:1.50, at least about 1:1.45:1.50, at least about 1:1.50:1.50, at least about 1:1.55:1.50, at least about 1:1.60:1.50, at least about 1:1.65:1.50, at least about 1:1.70:1.50, at least about 1:1.75:1.50, at least about 1:1.80:1.50, at least about 1:1.85:1.50, at least about 1:1.90:1.50, at least about 1:1.95:1.50, or at least about 1:2:1.50.

In a further embodiment, the ratio of PD-1 antagonist to PVRIG antagonist to CD96 antagonist may be at least about 1:0.25:1.55, at least about 1:0.30:1.55, at least about 1:0.35:1.55, at least about 1:0.40:1.55, at least about 1:0.45:1.55, at least about 1:0.50:1.55, at least about 1:0.55:1.55, at least about 1:0.60:1.55, at least about 1:0.65:1.55, at least about 1:0.70:1.55, at least about 1:0.75:1.55, at least about 1:0.80:1.55, at least about 1:0.85:1.55, at least about 1:0.90:1.55, at least about 1:0.95:1.55, at least about 1:1:1.55, at least about 1:1.05:1.55, at least about 1:1.10:1.55, at least about 1:1.15:1.55, at least about 1:1.20:1.55, at least about 1:1.25:1.55, at least about 1:1.30:1.55, at least about 1:1.35:1.55, at least about 1:1.40:1.55, at least about 1:1.45:1.55, at least about 1:1.50:1.55, at least about 1:1.55:1.55, at least about 1:1.60:1.55, at least about 1:1.65:1.55, at least about 1:1.70:1.55, at least about 1:1.75:1.55, at least about 1:1.80:1.55, at least about 1:1.85:1.55, at least about 1:1.90:1.55, at least about 1:1.95:1.55, or at least about 1:2:1.55.

In a further embodiment, the ratio of PD-1 antagonist to PVRIG antagonist to CD96 antagonist may be at least about 1:0.25:1.60, at least about 1:0.30:1.60, at least about 1:0.35:1.60, at least about 1:0.40:1.60, at least about 1:0.45:1.60, at least about 1:0.50:1.60, at least about 1:0.55:1.60, at least about 1:0.60:1.60, at least about 1:0.65:1.60, at least about 1:0.70:1.60, at least about 1:0.75:1.60, at least about 1:0.80:1.60, at least about 1:0.85:1.60, at least about 1:0.90:1.60, at least about 1:0.95:1.60, at least about 1:1:1.60, at least about 1:1.05:1.60, at least about 1:1.10:1.60, at least about 1:1.15:1.60, at least about 1:1.20:1.60, at least about 1:1.25:1.60, at least about 1:1.30:1.60, at least about 1:1.35:1.60, at least about 1:1.40:1.60, at least about 1:1.45:1.60, at least about 1:1.50:1.60, at least about 1:1.55:1.60, at least about 1:1.60:1.60, at least about 1:1.65:1.60, at least about 1:1.70:1.60, at least about 1:1.75:1.60, at least about 1:1.80:1.60, at least about 1:1.85:1.60, at least about 1:1.90:1.60, at least about 1:1.95:1.60, or at least about 1:2:1.60.

In a further embodiment, the ratio of PD-1 antagonist to PVRIG antagonist to CD96 antagonist may be at least about 1:0.25:1.65, at least about 1:0.30:1.65, at least about 1:0.35:1.65, at least about 1:0.40:1.65, at least about 1:0.45:1.65, at least about 1:0.50:1.65, at least about 1:0.55:1.65, at least about 1:0.60:1.65, at least about 1:0.65:1.65, at least about 1:0.70:1.65, at least about 1:0.75:1.65, at least about 1:0.80:1.65, at least about 1:0.85:1.65, at least about 1:0.90:1.65, at least about 1:0.95:1.65, at least about 1:1:1.65, at least about 1:1.05:1.65, at least about 1:1.10:1.65, at least about 1:1.15:1.65, at least about 1:1.20:1.65, at least about 1:1.25:1.65, at least about 1:1.30:1.65, at least about 1:1.35:1.65, at least about 1:1.40:1.65, at least about 1:1.45:1.65, at least about 1:1.50:1.65, at least about 1:1.55:1.65, at least about 1:1.60:1.65, at least about 1:1.65:1.65, at least about 1:1.70:1.65, at least about 1:1.75:1.65, at least about 1:1.80:1.65, at least about 1:1.85:1.65, at least about 1:1.90:1.65, at least about 1:1.95:1.65, or at least about 1:2:1.65.

In a further embodiment, the ratio of PD-1 antagonist to PVRIG antagonist to CD96 antagonist may be at least about 1:0.25:1.70, at least about 1:0.30:1.70, at least about 1:0.35:1.70, at least about 1:0.40:1.70, at least about 1:0.45:1.70, at least about 1:0.50:1.70, at least about 1:0.55:1.70, at least about 1:0.60:1.70, at least about 1:0.65:1.70, at least about 1:0.70:1.70, at least about 1:0.75:1.70, at least about 1:0.80:1.70, at least about 1:0.85:1.70, at least about 1:0.90:1.70, at least about 1:0.95:1.70, at least about 1:1:1.70, at least about 1:1.05:1.70, at least about 1:1.10:1.70, at least about 1:1.15:1.70, at least about 1:1.20:1.70, at least about 1:1.25:1.70, at least about 1:1.30:1.70, at least about 1:1.35:1.70, at least about 1:1.40:1.70, at least about 1:1.45:1.70, at least about 1:1.50:1.70, at least about 1:1.55:1.70, at least about 1:1.60:1.70, at least about 1:1.65:1.70, at least about 1:1.70:1.70, at least about 1:1.75:1.70, at least about 1:1.80:1.70, at least about 1:1.85:1.70, at least about 1:1.90:1.70, at least about 1:1.95:1.70, or at least about 1:2:1.70.

In a further embodiment, the ratio of PD-1 antagonist to PVRIG antagonist to CD96 antagonist may be at least about 1:0.25:1.75, at least about 1:0.30:1.75, at least about 1:0.35:1.75, at least about 1:0.40:1.75, at least about 1:0.45:1.75, at least about 1:0.50:1.75, at least about 1:0.55:1.75, at least about 1:0.60:1.75, at least about 1:0.65:1.75, at least about 1:0.70:1.75, at least about 1:0.75:1.75, at least about 1:0.80:1.75, at least about 1:0.85:1.75, at least about 1:0.90:1.75, at least about 1:0.95:1.75, at least about 1:1:1.75, at least about 1:1.05:1.75, at least about 1:1.10:1.75, at least about 1:1.15:1.75, at least about 1:1.20:1.75, at least about 1:1.25:1.75, at least about 1:1.30:1.75, at least about 1:1.35:1.75, at least about 1:1.40:1.75, at least about 1:1.45:1.75, at least about 1:1.50:1.75, at least about 1:1.55:1.75, at least about 1:1.60:1.75, at least about 1:1.65:1.75, at least about 1:1.70:1.75, at least about 1:1.75:1.75, at least about 1:1.80:1.75, at least about 1:1.85:1.75, at least about 1:1.90:1.75, at least about 1:1.95:1.75, or at least about 1:2:1.75.

In a further embodiment, the ratio of PD-1 antagonist to PVRIG antagonist to CD96 antagonist may be at least about 1:0.25:1.80, at least about 1:0.30:1.80, at least about 1:0.35:1.80, at least about 1:0.40:1.80, at least about 1:0.45:1.80, at least about 1:0.50:1.80, at least about 1:0.55:1.80, at least about 1:0.60:1.80, at least about 1:0.65:1.80, at least about 1:0.70:1.80, at least about 1:0.75:1.80, at least about 1:0.80:1.80, at least about 1:0.85:1.80, at least about 1:0.90:1.80, at least about 1:0.95:1.80, at least about 1:1:1.80, at least about 1:1.05:1.80, at least about 1:1.10:1.80, at least about 1:1.15:1.80, at least about 1:1.20:1.80, at least about 1:1.25:1.80, at least about 1:1.30:1.80, at least about 1:1.35:1.80, at least about 1:1.40:1.80, at least about 1:1.45:1.80, at least about 1:1.50:1.80, at least about 1:1.55:1.80, at least about 1:1.60:1.80, at least about 1:1.65:1.80, at least about 1:1.70:1.80, at least about 1:1.75:1.80, at least about 1:1.80:1.80, at least about 1:1.85:1.80, at least about 1:1.90:1.80, at least about 1:1.95:1.80, or at least about 1:2:1.80.

In a further embodiment, the ratio of PD-1 antagonist to PVRIG antagonist to CD96 antagonist may be at least about 1:0.25:1.85, at least about 1:0.30:1.85, at least about 1:0.35:1.85, at least about 1:0.40:1.85, at least about 1:0.45:1.85, at least about 1:0.50:1.85, at least about 1:0.55:1.85, at least about 1:0.60:1.85, at least about 1:0.65:1.85, at least about 1:0.70:1.85, at least about 1:0.75:1.85, at least about 1:0.80:1.85, at least about 1:0.85:1.85, at least about 1:0.90:1.85, at least about 1:0.95:1.85, at least about 1:1:1.85, at least about 1:1.05:1.85, at least about 1:1.10:1.85, at least about 1:1.15:1.85, at least about 1:1.20:1.85, at least about 1:1.25:1.85, at least about 1:1.30:1.85, at least about 1:1.35:1.85, at least about 1:1.40:1.85, at least about 1:1.45:1.85, at least about 1:1.50:1.85, at least about 1:1.55:1.85, at least about 1:1.60:1.85, at least about 1:1.65:1.85, at least about 1:1.70:1.85, at least about 1:1.75:1.85, at least about 1:1.80:1.85, at least about 1:1.85:1.85, at least about 1:1.90:1.85, at least about 1:1.95:1.85, or at least about 1:2:1.85.

In a further embodiment, the ratio of PD-1 antagonist to PVRIG antagonist to CD96 antagonist may be at least about 1:0.25:1.90, at least about 1:0.30:1.90, at least about 1:0.35:1.90, at least about 1:0.40:1.90, at least about 1:0.45:1.90, at least about 1:0.50:1.90, at least about 1:0.55:1.90, at least about 1:0.60:1.90, at least about 1:0.65:1.90, at least about 1:0.70:1.90, at least about 1:0.75:1.90, at least about 1:0.80:1.90, at least about 1:0.85:1.90, at least about 1:0.90:1.90, at least about 1:0.95:1.90, at least about 1:1:1.90, at least about 1:1.05:1.90, at least about 1:1.10:1.90, at least about 1:1.15:1.90, at least about 1:1.20:1.90, at least about 1:1.25:1.90, at least about 1:1.30:1.90, at least about 1:1.35:1.90, at least about 1:1.40:1.90, at least about 1:1.45:1.90, at least about 1:1.50:1.90, at least about 1:1.55:1.90, at least about 1:1.60:1.90, at least about 1:1.65:1.90, at least about 1:1.70:1.90, at least about 1:1.75:1.90, at least about 1:1.80:1.90, at least about 1:1.85:1.90, at least about 1:1.90:1.90, at least about 1:1.95:1.90, or at least about 1:2:1.90.

In a further embodiment, the ratio of PD-1 antagonist to PVRIG antagonist to CD96 antagonist may be at least about 1:0.25:1.95, at least about 1:0.30:1.95, at least about 1:0.35:1.95, at least about 1:0.40:1.95, at least about 1:0.45:1.95, at least about 1:0.50:1.95, at least about 1:0.55:1.95, at least about 1:0.60:1.95, at least about 1:0.65:1.95, at least about 1:0.70:1.95, at least about 1:0.75:1.95, at least about 1:0.80:1.95, at least about 1:0.85:1.95, at least about 1:0.90:1.95, at least about 1:0.95:1.95, at least about 1:1:1.95, at least about 1:1.05:1.95, at least about 1:1.10:1.95, at least about 1:1.15:1.95, at least about 1:1.20:1.95, at least about 1:1.25:1.95, at least about 1:1.30:1.95, at least about 1:1.35:1.95, at least about 1:1.40:1.95, at least about 1:1.45:1.95, at least about 1:1.50:1.95, at least about 1:1.55:1.95, at least about 1:1.60:1.95, at least about 1:1.65:1.95, at least about 1:1.70:1.95, at least about 1:1.75:1.95, at least about 1:1.80:1.95, at least about 1:1.85:1.95, at least about 1:1.90:1.95, at least about 1:1.95:1.95, or at least about 1:2:1.95.

In a further embodiment, the ratio of PD-1 antagonist to PVRIG antagonist to CD96 antagonist may be at least about 1:0.25:2, at least about 1:0.30:2, at least about 1:0.35:2, at least about 1:0.40:2, at least about 1:0.45:2, at least about 1:0.50:2, at least about 1:0.55:2, at least about 1:0.60:2, at least about 1:0.65:2, at least about 1:0.70:2, at least about 1:0.75:2, at least about 1:0.80:2, at least about 1:0.85:2, at least about 1:0.90:2, at least about 1:0.95:2, at least about 1:1:2, at least about 1:1.05:2, at least about 1:1.10:2, at least about 1:1.15:2, at least about 1:1.20:2, at least about 1:1.25:2, at least about 1:1.30:2, at least about 1:1.35:2, at least about 1:1.40:2, at least about 1:1.45:2, at least about 1:1.50:2, at least about 1:1.55:2, at least about 1:1.60:2, at least about 1:1.65:2, at least about 1:1.70:2, at least about 1:1.75:2, at least about 1:1.80:2, at least about 1:1.85:2, at least about 1:1.90:2, at least about 1:1.95:2, or at least about 1:2:2.

In a further embodiment, the ratio of CD96 antagonist to PVRIG antagonist to TIGIT antagonist may be at least about 1:0.25:0.25, at least about 1:0.30:0.30, at least about 1:0.35:0.35, at least about 1:0.40:0.40, at least about 1:0.45:0.45, at least about 1:0.50:0.50, at least about 1:0.55:0.55, at least about 1:0.60:0.60, at least about 1:0.65:0.65, at least about 1:0.70:0.70, at least about 1:0.75:0.75, at least about 1:0.80:0.80, at least about 1:0.85:0.85, at least about 1:0.90:0.90, at least about 1:0.95:0.95, at least about 1:1:1, at least about 1:1.05:1.05, at least about 1:1.10:1.10, at least about 1:1.15:1.15, at least about 1:1.20:1.20, at least about 1:1.25:1.25, at least about 1:1.30:1.30, at least about 1:1.35:1.35, at least about 1:1.40:1.40, at least about 1:1.45:1.45, at least about 1:1.50:1.50, at least about 1:1.55:1.55, at least about 1:1.60:1.60, at least about 1:1.65:1.65, at least about 1:1.70:1.70, at least about 1:1.75:1.75, at least about 1:1.80:1.80, at least about 1:1.85:1.85, at least about 1:1.90:1.90, at least about 1:1.95:1.95, or at least about 1:2:2.

In a further embodiment, the ratio of CD96 antagonist to PVRIG antagonist to TIGIT antagonist may be at least about 1:0.25:0.30, at least about 1:0.25:0.35, at least about 1:0.25:0.40, at least about 1:0.25:0.45, at least about 1:0.25:0.50, at least about 1:0.25:0.55, at least about 1:0.25:0.60, at least about 1:0.25:0.65, at least about 1:0.25:0.70, at least about 1:0.25:0.75, at least about 1:0.25:0.80, at least about 1:0.25:0.85, at least about 1:0.25:0.90, at least about 1:0.25:0.95, at least about 1:0.25:1, at least about 1:0.25:1.05, at least about 1:0.25:1.10, at least about 1:0.25:1.15, at least about 1:0.25:1.20, at least about 1:0.25:1.25, at least about 1:0.25:1.30, at least about 1:0.25:1.35, at least about 1:0.25:1.40, at least about 1:0.25:1.45, at least about 1:0.25:1.50, at least about 1:0.25:1.55, at least about 1:0.25:1.60, at least about 1:0.25:1.65, at least about 1:0.25:1.70, at least about 1:0.25:1.75, at least about 1:0.25:1.80, at least about 1:0.25:1.85, at least about 1:0.25:1.90, at least about 1:0.25:1.95, or at least about 1:0.25:2.

In a further embodiment, the ratio of CD96 antagonist to PVRIG antagonist to TIGIT antagonist may be at least about 1:0.30:0.25, at least about 1:0.30:0.30, at least about 1:0.30:0.35, at least about 1:0.30:0.40, at least about 1:0.30:0.45, at least about 1:0.30:0.50, at least about 1:0.30:0.55, at least about 1:0.30:0.60, at least about 1:0.30:0.65, at least about 1:0.30:0.70, at least about 1:0.30:0.75, at least about 1:0.30:0.80, at least about 1:0.30:0.85, at least about 1:0.30:0.90, at least about 1:0.30:0.95, at least about 1:0.30:1, at least about 1:0.30:1.05, at least about 1:0.30:1.10, at least about 1:0.30:1.15, at least about 1:0.30:1.20, at least about 1:0.30:1.25, at least about 1:0.30:1.30, at least about 1:0.30:1.35, at least about 1:0.30:1.40, at least about 1:0.30:1.45, at least about 1:0.30:1.50, at least about 1:0.30:1.55, at least about 1:0.30:1.60, at least about 1:0.30:1.65, at least about 1:0.30:1.70, at least about 1:0.30:1.75, at least about 1:0.30:1.80, at least about 1:0.30:1.85, at least about 1:0.30:1.90, at least about 1:0.30:1.95, or at least about 1:0.30:2.

In a further embodiment, the ratio of CD96 antagonist to PVRIG antagonist to TIGIT antagonist may be at least about 1:0.35:0.25, at least about 1:0.35:0.30, at least about 1:0.35:0.35, at least about 1:0.35:0.40, at least about 1:0.35:0.45, at least about 1:0.35:0.50, at least about 1:0.35:0.55, at least about 1:0.35:0.60, at least about 1:0.35:0.65, at least about 1:0.35:0.70, at least about 1:0.35:0.75, at least about 1:0.35:0.80, at least about 1:0.35:0.85, at least about 1:0.35:0.90, at least about 1:0.35:0.95, at least about 1:0.35:1, at least about 1:0.35:1.05, at least about 1:0.35:1.10, at least about 1:0.35:1.15, at least about 1:0.35:1.20, at least about 1:0.35:1.25, at least about 1:0.35:1.30, at least about 1:0.35:1.35, at least about 1:0.35:1.40, at least about 1:0.35:1.45, at least about 1:0.35:1.50, at least about 1:0.35:1.55, at least about 1:0.35:1.60, at least about 1:0.35:1.65, at least about 1:0.35:1.70, at least about 1:0.35:1.75, at least about 1:0.35:1.80, at least about 1:0.35:1.85, at least about 1:0.35:1.90, at least about 1:0.35:1.95, or at least about 1:0.35:2.

In a further embodiment, the ratio of CD96 antagonist to PVRIG antagonist to TIGIT antagonist may be at least about 1:0.40:0.25, at least about 1:0.40:0.30, at least about 1:0.40:0.35, at least about 1:0.40:0.40, at least about 1:0.40:0.45, at least about 1:0.40:0.50, at least about 1:0.40:0.55, at least about 1:0.40:0.60, at least about 1:0.40:0.65, at least about 1:0.40:0.70, at least about 1:0.40:0.75, at least about 1:0.40:0.80, at least about 1:0.40:0.85, at least about 1:0.40:0.90, at least about 1:0.40:0.95, at least about 1:0.40:1, at least about 1:0.40:1.05, at least about 1:0.40:1.10, at least about 1:0.40:1.15, at least about 1:0.40:1.20, at least about 1:0.40:1.25, at least about 1:0.40:1.30, at least about 1:0.40:1.35, at least about 1:0.40:1.40, at least about 1:0.40:1.45, at least about 1:0.40:1.50, at least about 1:0.40:1.55, at least about 1:0.40:1.60, at least about 1:0.40:1.65, at least about 1:0.40:1.70, at least about 1:0.40:1.75, at least about 1:0.40:1.80, at least about 1:0.40:1.85, at least about 1:0.40:1.90, at least about 1:0.40:1.95, or at least about 1:0.40:2.

In a further embodiment, the ratio of CD96 antagonist to PVRIG antagonist to TIGIT antagonist may be at least about 1:0.45:0.25, at least about 1:0.45:0.30, at least about 1:0.45:0.35, at least about 1:0.45:0.40, at least about 1:0.45:0.45, at least about 1:0.45:0.50, at least about 1:0.45:0.55, at least about 1:0.45:0.60, at least about 1:0.45:0.65, at least about 1:0.45:0.70, at least about 1:0.45:0.75, at least about 1:0.45:0.80, at least about 1:0.45:0.85, at least about 1:0.45:0.90, at least about 1:0.45:0.95, at least about 1:0.45:1, at least about 1:0.45:1.05, at least about 1:0.45:1.10, at least about 1:0.45:1.15, at least about 1:0.45:1.20, at least about 1:0.45:1.25, at least about 1:0.45:1.30, at least about 1:0.45:1.35, at least about 1:0.45:1.40, at least about 1:0.45:1.45, at least about 1:0.45:1.50, at least about 1:0.45:1.55, at least about 1:0.45:1.60, at least about 1:0.45:1.65, at least about 1:0.45:1.70, at least about 1:0.45:1.75, at least about 1:0.45:1.80, at least about 1:0.45:1.85, at least about 1:0.45:1.90, at least about 1:0.45:1.95, or at least about 1:0.45:2.

In a further embodiment, the ratio of CD96 antagonist to PVRIG antagonist to TIGIT antagonist may be at least about 1:0.50:0.25, at least about 1:0.50:0.30, at least about 1:0.50:0.35, at least about 1:0.50:0.40, at least about 1:0.50:0.45, at least about 1:0.50:0.50, at least about 1:0.50:0.55, at least about 1:0.50:0.60, at least about 1:0.50:0.65, at least about 1:0.50:0.70, at least about 1:0.50:0.75, at least about 1:0.50:0.80, at least about 1:0.50:0.85, at least about 1:0.50:0.90, at least about 1:0.50:0.95, at least about 1:0.50:1, at least about 1:0.50:1.05, at least about 1:0.50:1.10, at least about 1:0.50:1.15, at least about 1:0.50:1.20, at least about 1:0.50:1.25, at least about 1:0.50:1.30, at least about 1:0.50:1.35, at least about 1:0.50:1.40, at least about 1:0.50:1.45, at least about 1:0.50:1.50, at least about 1:0.50:1.55, at least about 1:0.50:1.60, at least about 1:0.50:1.65, at least about 1:0.50:1.70, at least about 1:0.50:1.75, at least about 1:0.50:1.80, at least about 1:0.50:1.85, at least about 1:0.50:1.90, at least about 1:0.50:1.95, or at least about 1:0.50:2.

In a further embodiment, the ratio of CD96 antagonist to PVRIG antagonist to TIGIT antagonist may be at least about 1:0.55:0.25, at least about 1:0.55:0.30, at least about 1:0.55:0.35, at least about 1:0.55:0.40, at least about 1:0.55:0.45, at least about 1:0.55:0.50, at least about 1:0.55:0.55, at least about 1:0.55:0.60, at least about 1:0.55:0.65, at least about 1:0.55:0.70, at least about 1:0.55:0.75, at least about 1:0.55:0.80, at least about 1:0.55:0.85, at least about 1:0.55:0.90, at least about 1:0.55:0.95, at least about 1:0.55:1, at least about 1:0.55:1.05, at least about 1:0.55:1.10, at least about 1:0.55:1.15, at least about 1:0.55:1.20, at least about 1:0.55:1.25, at least about 1:0.55:1.30, at least about 1:0.55:1.35, at least about 1:0.55:1.40, at least about 1:0.55:1.45, at least about 1:0.55:1.50, at least about 1:0.55:1.55, at least about 1:0.55:1.60, at least about 1:0.55:1.65, at least about 1:0.55:1.70, at least about 1:0.55:1.75, at least about 1:0.55:1.80, at least about 1:0.55:1.85, at least about 1:0.55:1.90, 1:0.55:1.95, or at least about 1:0.55:2.

In a further embodiment, the ratio of CD96 antagonist to PVRIG antagonist to TIGIT antagonist may be at least about 1:0.60:0.25, at least about 1:0.60:0.30, at least about 1:0.60:0.35, at least about 1:0.60:0.40, at least about 1:0.60:0.45, at least about 1:0.60:0.50, at least about 1:0.60:0.55, at least about 1:0.60:0.60, at least about 1:0.60:0.65, at least about 1:0.60:0.70, at least about 1:0.60:0.75, at least about 1:0.60:0.80, at least about 1:0.60:0.85, at least about 1:0.60:0.90, at least about 1:0.60:0.95, at least about 1:0.60:1, at least about 1:0.60:1.05, at least about 1:0.60:1.10, at least about 1:0.60:1.15, at least about 1:0.60:1.20, at least about 1:0.60:1.25, at least about 1:0.60:1.30, at least about 1:0.60:1.35, at least about 1:0.60:1.40, at least about 1:0.60:1.45, at least about 1:0.60:1.50, at least about 1:0.60:1.55, at least about 1:0.60:1.60, at least about 1:0.60:1.65, at least about 1:0.60:1.70, at least about 1:0.60:1.75, at least about 1:0.60:1.80, at least about 1:0.60:1.85, at least about 1:0.60:1.90, at least about 1:0.60:1.95, or at least about 1:0.60:2.

In a further embodiment, the ratio of CD96 antagonist to PVRIG antagonist to TIGIT antagonist may be at least about 1:0.65:0.25, at least about 1:0.65:0.30, at least about 1:0.65:0.35, at least about 1:0.65:0.40, at least about 1:0.65:0.45, at least about 1:0.65:0.50, at least about 1:0.65:0.55, at least about 1:0.65:0.60, at least about 1:0.65:0.65, at least about 1:0.65:0.70, at least about 1:0.65:0.75, at least about 1:0.65:0.80, at least about 1:0.65:0.85, at least about 1:0.65:0.90, at least about 1:0.65:0.95, at least about 1:0.65:1, at least about 1:0.65:1.05, at least about 1:0.65:1.10, at least about 1:0.65:1.15, at least about 1:0.65:1.20, at least about 1:0.65:1.25, at least about 1:0.65:1.30, at least about 1:0.65:1.35, at least about 1:0.65:1.40, at least about 1:0.65:1.45, at least about 1:0.65:1.50, at least about 1:0.65:1.55, at least about 1:0.65:1.60, at least about 1:0.65:1.65, at least about 1:0.65:1.70, at least about 1:0.65:1.75, at least about 1:0.65:1.80, at least about 1:0.65:1.85, at least about 1:0.65:1.90, at least about 1:0.65:1.95, or at least about 1:0.65:2.

In a further embodiment, the ratio of CD96 antagonist to PVRIG antagonist to TIGIT antagonist may be at least about 1:0.70:0.25, at least about 1:0.70:0.30, at least about 1:0.70:0.35, at least about 1:0.70:0.40, at least about 1:0.70:0.45, at least about 1:0.70:0.50, at least about 1:0.70:0.55, at least about 1:0.70:0.60, at least about 1:0.70:0.65, at least about 1:0.70:0.70, at least about 1:0.70:0.75, at least about 1:0.70:0.80, at least about 1:0.70:0.85, at least about 1:0.70:0.90, at least about 1:0.70:0.95, at least about 1:0.70:1, at least about 1:0.70:1.05, at least about 1:0.70:1.10, at least about 1:0.70:1.15, at least about 1:0.70:1.20, at least about 1:0.70:1.25, at least about 1:0.70:1.30, at least about 1:0.70:1.35, at least about 1:0.70:1.40, at least about 1:0.70:1.45, at least about 1:0.70:1.50, at least about 1:0.70:1.55, at least about 1:0.70:1.60, at least about 1:0.70:1.65, at least about 1:0.70:1.70, at least about 1:0.70:1.75, at least about 1:0.70:1.80, at least about 1:0.70:1.85, at least about 1:0.70:1.90, at least about 1:0.70:1.95, or at least about 1:0.70:2.

In a further embodiment, the ratio of CD96 antagonist to PVRIG antagonist to TIGIT antagonist may be at least about 1:0.75:0.25, at least about 1:0.75:0.30, at least about 1:0.75:0.35, at least about 1:0.75:0.40, at least about 1:0.75:0.45, at least about 1:0.75:0.50, at least about 1:0.75:0.55, at least about 1:0.75:0.60, at least about 1:0.75:0.65, at least about 1:0.75:0.70, at least about 1:0.75:0.75, at least about 1:0.75:0.80, at least about 1:0.75:0.85, at least about 1:0.75:0.90, at least about 1:0.75:0.95, at least about 1:0.75:1, at least about 1:0.75:1.05, at least about 1:0.75:1.10, at least about 1:0.75:1.15, at least about 1:0.75:1.20, at least about 1:0.75:1.25, at least about 1:0.75:1.30, at least about 1:0.75:1.35, at least about 1:0.75:1.40, at least about 1:0.75:1.45, at least about 1:0.75:1.50, at least about 1:0.75:1.55, at least about 1:0.75:1.60, at least about 1:0.75:1.65, at least about 1:0.75:1.70, at least about 1:0.75:1.75, at least about 1:0.75:1.80, at least about 1:0.75:1.85, at least about 1:0.75:1.90, at least about 1:0.75:1.95, or at least about 1:0.75:2.

In a further embodiment, the ratio of CD96 antagonist to PVRIG antagonist to TIGIT antagonist may be at least about 1:0.80:0.25, at least about 1:0.80:0.30, at least about 1:0.80:0.35, at least about 1:0.80:0.40, at least about 1:0.80:0.45, at least about 1:0.80:0.50, at least about 1:0.80:0.55, at least about 1:0.80:0.60, at least about 1:0.80:0.65, at least about 1:0.80:0.70, at least about 1:0.80:0.75, at least about 1:0.80:0.80, at least about 1:0.80:0.85, at least about 1:0.80:0.90, at least about 1:0.80:0.95, at least about 1:0.80:1, at least about 1:0.80:1.05, at least about 1:0.80:1.10, at least about 1:0.80:1.15, at least about 1:0.80:1.20, at least about 1:0.80:1.25, at least about 1:0.80:1.30, at least about 1:0.80:1.35, at least about 1:0.80:1.40, at least about 1:0.80:1.45, at least about 1:0.80:1.50, at least about 1:0.80:1.55, at least about 1:0.80:1.60, at least about 1:0.80:1.65, at least about 1:0.80:1.70, at least about 1:0.80:1.75, at least about 1:0.80:1.80, at least about 1:0.80:1.85, at least about 1:0.80:1.90, at least about 1:0.80:1.95, or at least about 1:0.80:2.

In a further embodiment, the ratio of CD96 antagonist to PVRIG antagonist to TIGIT antagonist may be at least about 1:0.85:0.25, at least about 1:0.85:0.30, at least about 1:0.85:0.35, at least about 1:0.85:0.40, at least about 1:0.85:0.45, at least about 1:0.85:0.50, at least about 1:0.85:0.55, at least about 1:0.85:0.60, at least about 1:0.85:0.65, at least about 1:0.85:0.70, at least about 1:0.85:0.75, at least about 1:0.85:0.80, at least about 1:0.85:0.85, at least about 1:0.85:0.90, at least about 1:0.85:0.95, at least about 1:0.85:1, at least about 1:0.85:1.05, at least about 1:0.85:1.10, at least about 1:0.85:1.15, at least about 1:0.85:1.20, at least about 1:0.85:1.25, at least about 1:0.85:1.30, at least about 1:0.85:1.35, at least about 1:0.85:1.40, at least about 1:0.85:1.45, at least about 1:0.85:1.50, at least about 1:0.85:1.55, at least about 1:0.85:1.60, at least about 1:0.85:1.65, at least about 1:0.85:1.70, at least about 1:0.85:1.75, at least about 1:0.85:1.80, at least about 1:0.85:1.85, at least about 1:0.85:1.90, at least about 1:0.85:1.95, or at least about 1:0.85:2.

In a further embodiment, the ratio of CD96 antagonist to PVRIG antagonist to TIGIT antagonist may be at least about 1:0.90:0.25, at least about 1:0.90:0.30, at least about 1:0.90:0.35, at least about 1:0.90:0.40, at least about 1:0.90:0.45, at least about 1:0.90:0.50, at least about 1:0.90:0.55, at least about 1:0.90:0.60, at least about 1:0.90:0.65, at least about 1:0.90:0.70, at least about 1:0.90:0.75, at least about 1:0.90:0.80, at least about 1:0.90:0.85, at least about 1:0.90:0.90, at least about 1:0.90:0.95, at least about 1:0.90:1, at least about 1:0.90:1.05, at least about 1:0.90:1.10, at least about 1:0.90:1.15, at least about 1:0.90:1.20, at least about 1:0.90:1.25, at least about 1:0.90:1.30, at least about 1:0.90:1.35, at least about 1:0.90:1.40, at least about 1:0.90:1.45, at least about 1:0.90:1.50, at least about 1:0.90:1.55, at least about 1:0.90:1.60, at least about 1:0.90:1.65, at least about 1:0.90:1.70, at least about 1:0.90:1.75, at least about 1:0.90:1.80, at least about 1:0.90:1.85, at least about 1:0.90:1.90, at least about 1:0.90:1.95, or at least about 1:0.90:2.

In a further embodiment, the ratio of CD96 antagonist to PVRIG antagonist to TIGIT antagonist may be at least about 1:0.95:0.25, at least about 1:0.95:0.30, at least about 1:0.95:0.35, at least about 1:0.95:0.40, at least about 1:0.95:0.45, at least about 1:0.95:0.50, at least about 1:0.95:0.55, at least about 1:0.95:0.60, at least about 1:0.95:0.65, at least about 1:0.95:0.70, at least about 1:0.95:0.75, at least about 1:0.95:0.80, at least about 1:0.95:0.85, at least about 1:0.95:0.90, at least about 1:0.95:0.95, at least about 1:0.95:1, at least about 1:0.95:1.05, at least about 1:0.95:1.10, at least about 1:0.95:1.15, at least about 1:0.95:1.20, at least about 1:0.95:1.25, at least about 1:0.95:1.30, at least about 1:0.95:1.35, at least about 1:0.95:1.40, at least about 1:0.95:1.45, at least about 1:0.95:1.50, at least about 1:0.95:1.55, at least about 1:0.95:1.60, at least about 1:0.95:1.65, at least about 1:0.95:1.70, at least about 1:0.95:1.75, at least about 1:0.95:1.80, at least about 1:0.95:1.85, at least about 1:0.95:1.90, at least about 1:0.95:1.95, or at least about 1:0.95:2.

In a further embodiment, the ratio of CD96 antagonist to PVRIG antagonist to TIGIT antagonist may be at least about 1:1:0.25, at least about 1:1:0.30, at least about 1:1:0.35, at least about 1:1:0.40, at least about 1:1:0.45, at least about 1:1:0.50, at least about 1:1:0.55, at least about 1:1:0.60, at least about 1:1:0.65, at least about 1:1:0.70, at least about 1:1:0.75, at least about 1:1:0.80, at least about 1:1:0.85, at least about 1:1:0.90, at least about 1:1:0.95, at least about 1:1:1, at least about 1:1:1.05, at least about 1:1:1.10, at least about 1:1:1.15, at least about 1:1:1.20, at least about 1:1:1.25, at least about 1:1:1.30, at least about 1:1:1.35, at least about 1:1:1.40, at least about 1:1:1.45, at least about 1:1:1.50, at least about 1:1:1.55, at least about 1:1:1.60, at least about 1:1:1.65, at least about 1:1:1.70, at least about 1:1:1.75, at least about 1:1:1.80, at least about 1:1:1.85, at least about 1:1:1.90, at least about 1:1:1.95, or at least about 1:1:2.

In a further embodiment, the ratio of CD96 antagonist to PVRIG antagonist to TIGIT antagonist may be at least about 1:1.05:0.25, at least about 1:1.05:0.30, at least about 1:1.05:0.35, at least about 1:1.05:0.40, at least about 1:1.05:0.45, at least about 1:1.05:0.50, at least about 1:1.05:0.55, at least about 1:1.05:0.60, at least about 1:1.05:0.65, at least about 1:1.05:0.70, at least about 1:1.05:0.75, at least about 1:1.05:0.80, at least about 1:1.05:0.85, at least about 1:1.05:0.90, at least about 1:1.05:0.95, at least about 1:1.05:1, at least about 1:1.05:1.05, at least about 1:1.05:1.10, at least about 1:1.05:1.15, at least about 1:1.05:1.20, at least about 1:1.05:1.25, at least about 1:1.05:1.30, at least about 1:1.05:1.35, at least about 1:1.05:1.40, at least about 1:1.05:1.45, at least about 1:1.05:1.50, at least about 1:1.05:1.55, at least about 1:1.05:1.60, at least about 1:1.05:1.65, at least about 1:1.05:1.70, at least about 1:1.05:1.75, at least about 1:1.05:1.80, at least about 1:1.05:1.85, at least about 1:1.05:1.90, at least about 1:1.05:1.95, or at least about 1:1.05:2.

In a further embodiment, the ratio of CD96 antagonist to PVRIG antagonist to TIGIT antagonist may be at least about 1:1.10:0.25, at least about 1:1.10:0.30, at least about 1:1.10:0.35, at least about 1:1.10:0.40, at least about 1:1.10:0.45, at least about 1:1.10:0.50, at least about 1:1.10:0.55, at least about 1:1.10:0.60, at least about 1:1.10:0.65, at least about 1:1.10:0.70, at least about 1:1.10:0.75, at least about 1:1.10:0.80, at least about 1:1.10:0.85, at least about 1:1.10:0.90, at least about 1:1.10:0.95, at least about 1:1.10:1, at least about 1:1.10:1.05, at least about 1:1.10:1.10, at least about 1:1.10:1.15, at least about 1:1.10:1.20, at least about 1:1.10:1.25, at least about 1:1.10:1.30, at least about 1:1.10:1.35, at least about 1:1.10:1.40, at least about 1:1.10:1.45, at least about 1:1.10:1.50, at least about 1:1.10:1.55, at least about 1:1.10:1.60, at least about 1:1.10:1.65, at least about 1:1.10:1.70, at least about 1:1.10:1.75, at least about 1:1.10:1.80, at least about 1:1.10:1.85, at least about 1:1.10:1.90, at least about 1:1.10:1.95, or at least about 1:1.10:2.

In a further embodiment, the ratio of CD96 antagonist to PVRIG antagonist to TIGIT antagonist may be at least about 1:1.15:0.25, at least about 1:1.15:0.30, at least about 1:1.15:0.35, at least about 1:1.15:0.40, at least about 1:1.15:0.45, at least about 1:1.15:0.50, at least about 1:1.15:0.55, at least about 1:1.15:0.60, at least about 1:1.15:0.65, at least about 1:1.15:0.70, at least about 1:1.15:0.75, at least about 1:1.15:0.80, at least about 1:1.15:0.85, at least about 1:1.15:0.90, at least about 1:1.15:0.95, at least about 1:1.15:1, at least about 1:1.15:1.05, at least about 1:1.15:1.10, at least about 1:1.15:1.15, at least about 1:1.15:1.20, at least about 1:1.15:1.25, at least about 1:1.15:1.30, at least about 1:1.15:1.35, at least about 1:1.15:1.40, at least about 1:1.15:1.45, at least about 1:1.15:1.50, at least about 1:1.15:1.55, at least about 1:1.15:1.60, at least about 1:1.15:1.65, at least about 1:1.15:1.70, at least about 1:1.15:1.75, at least about 1:1.15:1.80, at least about 1:1.15:1.85, at least about 1:1.15:1.90, at least about 1:1.15:1.95, or at least about 1:1.15:2.

In a further embodiment, the ratio of CD96 antagonist to PVRIG antagonist to TIGIT antagonist may be at least about 1:1.20:0.25, at least about 1:1.20:0.30, at least about 1:1.20:0.35, at least about 1:1.20:0.40, at least about 1:1.20:0.45, at least about 1:1.20:0.50, at least about 1:1.20:0.55, at least about 1:1.20:0.60, at least about 1:1.20:0.65, at least about 1:1.20:0.70, at least about 1:1.20:0.75, at least about 1:1.20:0.80, at least about 1:1.20:0.85, at least about 1:1.20:0.90, at least about 1:1.20:0.95, at least about 1:1.20:1, at least about 1:1.20:1.05, at least about 1:1.20:1.10, at least about 1:1.20:1.15, at least about 1:1.20:1.20, at least about 1:1.20:1.25, at least about 1:1.20:1.30, at least about 1:1.20:1.35, at least about 1:1.20:1.40, at least about 1:1.20:1.45, at least about 1:1.20:1.50, at least about 1:1.20:1.55, at least about 1:1.20:1.60, at least about 1:1.20:1.65, at least about 1:1.20:1.70, at least about 1:1.20:1.75, at least about 1:1.20:1.80, at least about 1:1.20:1.85, at least about 1:1.20:1.90, at least about 1:1.20:1.95, or at least about 1:1.20:2.

In a further embodiment, the ratio of CD96 antagonist to PVRIG antagonist to TIGIT antagonist may be at least about 1:1.25:0.25, at least about 1:1.25:0.30, at least about 1:1.25:0.35, at least about 1:1.25:0.40, at least about 1:1.25:0.45, at least about 1:1.25:0.50, at least about 1:1.25:0.55, at least about 1:1.25:0.60, at least about 1:1.25:0.65, at least about 1:1.25:0.70, at least about 1:1.25:0.75, at least about 1:1.25:0.80, at least about 1:1.25:0.85, at least about 1:1.25:0.90, at least about 1:1.25:0.95, at least about 1:1.25:1, at least about 1:1.25:1.05, at least about 1:1.25:1.10, at least about 1:1.25:1.15, at least about 1:1.25:1.20, at least about 1:1.25:1.25, at least about 1:1.25:1.30, at least about 1:1.25:1.35, at least about 1:1.25:1.40, at least about 1:1.25:1.45, at least about 1:1.25:1.50, at least about 1:1.25:1.55, at least about 1:1.25:1.60, at least about 1:1.25:1.65, at least about 1:1.25:1.70, at least about 1:1.25:1.75, at least about 1:1.25:1.80, at least about 1:1.25:1.85, at least about 1:1.25:1.90, at least about 1:1.25:1.95, or at least about 1:1.25:2.

In a further embodiment, the ratio of CD96 antagonist to PVRIG antagonist to TIGIT antagonist may be at least about 1:1.30:0.25, at least about 1:1.30:0.30, at least about 1:1.30:0.35, at least about 1:1.30:0.40, at least about 1:1.30:0.45, at least about 1:1.30:0.50, at least about 1:1.30:0.55, at least about 1:1.30:0.60, at least about 1:1.30:0.65, at least about 1:1.30:0.70, at least about 1:1.30:0.75, at least about 1:1.30:0.80, at least about 1:1.30:0.85, at least about 1:1.30:0.90, at least about 1:1.30:0.95, at least about 1:1.30:1, at least about 1:1.30:1.05, at least about 1:1.30:1.10, at least about 1:1.30:1.15, at least about 1:1.30:1.20, at least about 1:1.30:1.25, at least about 1:1.30:1.30, at least about 1:1.30:1.35, at least about 1:1.30:1.40, at least about 1:1.30:1.45, at least about 1:1.30:1.50, at least about 1:1.30:1.55, at least about 1:1.30:1.60, at least about 1:1.30:1.65, at least about 1:1.30:1.70, at least about 1:1.30:1.75, at least about 1:1.30:1.80, at least about 1:1.30:1.85, at least about 1:1.30:1.90, at least about 1:1.30:1.95, or at least about 1:1.30:2.

In a further embodiment, the ratio of CD96 antagonist to PVRIG antagonist to TIGIT antagonist may be at least about 1:1.35:0.25, at least about 1:1.35:0.30, at least about 1:1.35:0.35, at least about 1:1.35:0.40, at least about 1:1.35:0.45, at least about 1:1.35:0.50, at least about 1:1.35:0.55, at least about 1:1.35:0.65, at least about 1:1.35:0.70, at least about 1:1.35:0.75, at least about 1:1.35:0.80, at least about 1:1.35:0.85, at least about 1:1.35:0.90, at least about 1:1.35:0.95, at least about 1:1.35:1, at least about 1:1.35:1.05, at least about 1:1.35:1.10, at least about 1:1.35:1.15, at least about 1:1.35:1.20, at least about 1:1.35:1.25, at least about 1:1.35:1.30, at least about 1:1.35:1.35, at least about 1:1.35:1.40, at least about 1:1.35:1.45, at least about 1:1.35:1.50, at least about 1:1.35:1.55, at least about 1:1.35:1.60, at least about 1:1.35:1.65, at least about 1:1.35:1.70, at least about 1:1.35:1.75, at least about 1:1.35:1.80, at least about 1:1.35:1.85, at least about 1:1.35:1.90, at least about 1:1.35:1.95, or at least about 1:1.35:2.

In a further embodiment, the ratio of CD96 antagonist to PVRIG antagonist to TIGIT antagonist may be at least about 1:1.40:0.25, at least about 1:1.40:0.30, at least about 1:1.40:0.35, at least about 1:1.40:0.40, at least about 1:1.40:0.45, at least about 1:1.40:0.50, at least about 1:1.40:0.55, at least about 1:1.40:0.60, at least about 1:1.40:0.65, at least about 1:1.40:0.70, at least about 1:1.40:0.75, at least about 1:1.40:0.80, at least about 1:1.40:0.85, at least about 1:1.40:0.90, at least about 1:1.40:0.95, at least about 1:1.40:1, at least about 1:1.40:1.05, at least about 1:1.40:1.10, at least about 1:1.40:1.15, at least about 1:1.40:1.20, at least about 1:1.40:1.25, at least about 1:1.40:1.30, at least about 1:1.40:1.35, at least about 1:1.40:1.40, at least about 1:1.40:1.45, at least about 1:1.40:1.50, at least about 1:1.40:1.55, at least about 1:1.40:1.60, at least about 1:1.40:1.65, at least about 1:1.40:1.70, at least about 1:1.40:1.75, at least about 1:1.40:1.80, at least about 1:1.40:1.85, at least about 1:1.40:1.90, at least about 1:1.40:1.95, or at least about 1:1.40:2.

In a further embodiment, the ratio of CD96 antagonist to PVRIG antagonist to TIGIT antagonist may be at least about 1:1.45:0.25, at least about 1:1.45:0.30, at least about 1:1.45:0.35, at least about 1:1.45:0.40, at least about 1:1.45:0.45, at least about 1:1.45:0.50, at least about 1:1.45:0.55, at least about 1:1.45:0.60, at least about 1:1.45:0.65, at least about 1:1.45:0.70, at least about 1:1.45:0.75, at least about 1:1.45:0.80, at least about 1:1.45:0.85, at least about 1:1.45:0.90, at least about 1:1.45:0.95, at least about 1:1.45:1, at least about 1:1.45:1.05, at least about 1:1.45:1.10, at least about 1:1.45:1.15, at least about 1:1.45:1.20, at least about 1:1.45:1.25, at least about 1:1.45:1.30, at least about 1:1.45:1.35, at least about 1:1.45:1.40, at least about 1:1.45:1.45, at least about 1:1.45:1.50, at least about 1:1.45:1.55, at least about 1:1.45:1.60, at least about 1:1.45:1.65, at least about 1:1.45:1.70, at least about 1:1.45:1.75, at least about 1:1.45:1.80, at least about 1:1.45:1.85, at least about 1:1.45:1.90, at least about 1:1.45:1.95, or at least about 1:1.45:2.

In a further embodiment, the ratio of CD96 antagonist to PVRIG antagonist to TIGIT antagonist may be at least about 1:1.50:0.25, at least about 1:1.50:0.30, at least about 1:1.50:0.35, at least about 1:1.50:0.40, at least about 1:1.50:0.45, at least about 1:1.50:0.50, at least about 1:1.50:0.55, at least about 1:1.50:0.60, at least about 1:1.50:0.65, at least about 1:1.50:0.70, at least about 1:1.50:0.75, at least about 1:1.50:0.80, at least about 1:1.50:0.85, at least about 1:1.50:0.90, at least about 1:1.50:0.95, at least about 1:1.50:1, at least about 1:1.50:1.05, at least about 1:1.50:1.10, at least about 1:1.50:1.15, at least about 1:1.50:1.20, at least about 1:1.50:1.25, at least about 1:1.50:1.30, at least about 1:1.50:1.35, at least about 1:1.50:1.40, at least about 1:1.50:1.45, at least about 1:1.50:1.50, at least about 1:1.50:1.55, at least about 1:1.50:1.60, at least about 1:1.50:1.65, at least about 1:1.50:1.70, at least about 1:1.50:1.75, at least about 1:1.50:1.80, at least about 1:1.50:1.85, at least about 1:1.50:1.90, at least about 1:1.50:1.95, or at least about 1:1.50:2.

In a further embodiment, the ratio of CD96 antagonist to PVRIG antagonist to TIGIT antagonist may be at least about 1:1.55:0.25, at least about 1:1.55:0.30, at least about 1:1.55:0.35, at least about 1:1.55:0.40, at least about 1:1.55:0.45, at least about 1:1.55:0.50, at least about 1:1.55:0.55, at least about 1:1.55:0.60, at least about 1:1.55:0.65, at least about 1:1.55:0.70, at least about 1:1.55:0.75, at least about 1:1.55:0.80, at least about 1:1.55:0.85, at least about 1:1.55:0.90, at least about 1:1.55:0.95, at least about 1:1.55:1, at least about 1:1.55:1.05, at least about 1:1.55:1.10, at least about 1:1.55:1.15, at least about 1:1.55:1.20, at least about 1:1.55:1.25, at least about 1:1.55:1.30, at least about 1:1.55:1.35, at least about 1:1.55:1.40, at least about 1:1.55:1.45, at least about 1:1.55:1.50, at least about 1:1.55:1.55, at least about 1:1.55:1.60, at least about 1:1.55:1.65, at least about 1:1.55:1.70, at least about 1:1.55:1.75, at least about 1:1.55:1.80, at least about 1:1.55:1.85, at least about 1:1.55:1.90, at least about 1:1.55:1.95, or at least about 1:1.55:2.

In a further embodiment, the ratio of CD96 antagonist to PVRIG antagonist to TIGIT antagonist may be at least about 1:1.60:0.25, at least about 1:1.60:0.30, at least about 1:1.60:0.35, at least about 1:1.60:0.40, at least about 1:1.60:0.45, at least about 1:1.60:0.50, at least about 1:1.60:0.55, at least about 1:1.60:0.60, at least about 1:1.60:0.65, at least about 1:1.60:0.70, at least about 1:1.60:0.75, at least about 1:1.60:0.80, at least about 1:1.60:0.85, at least about 1:1.60:0.90, at least about 1:1.60:0.95, at least about 1:1.60:1, at least about 1:1.60:1.05, at least about 1:1.60:1.10, at least about 1:1.60:1.15, at least about 1:1.60:1.20, at least about 1:1.60:1.25, at least about 1:1.60:1.30, at least about 1:1.60:1.35, at least about 1:1.60:1.40, at least about 1:1.60:1.45, at least about 1:1.60:1.50, at least about 1:1.60:1.55, at least about 1:1.60:1.60, at least about 1:1.60:1.65, at least about 1:1.60:1.70, at least about 1:1.60:1.75, at least about 1:1.60:1.80, at least about 1:1.60:1.85, at least about 1:1.60:1.90, at least about 1:1.60:1.95, or at least about 1:1.60:2.

In a further embodiment, the ratio of CD96 antagonist to PVRIG antagonist to TIGIT antagonist may be at least about 1:1.65:0.25, at least about 1:1.65:0.30, at least about 1:1.65:0.35, at least about 1:1.65:0.40, at least about 1:1.65:0.45, at least about 1:1.65:0.50, at least about 1:1.65:0.55, at least about 1:1.65:0.60, at least about 1:1.65:0.65, at least about 1:1.65:0.70, at least about 1:1.65:0.75, at least about 1:1.65:0.80, at least about 1:1.65:0.85, at least about 1:1.65:0.90, at least about 1:1.65:0.95, at least about 1:1.65:1, at least about 1:1.65:1.05, at least about 1:1.65:1.10, at least about 1:1.65:1.15, at least about 1:1.65:1.20, at least about 1:1.65:1.25, at least about 1:1.65:1.30, at least about 1:1.65:1.35, at least about 1:1.65:1.40, at least about 1:1.65:1.45, at least about 1:1.65:1.50, at least about 1:1.65:1.55, at least about 1:1.65:1.60, at least about 1:1.65:1.65, at least about 1:1.65:1.70, at least about 1:1.65:1.75, at least about 1:1.65:1.80, at least about 1:1.65:1.85, at least about 1:1.65:1.90, at least about 1:1.65:1.95, or at least about 1:1.65:2.

In a further embodiment, the ratio of CD96 antagonist to PVRIG antagonist to TIGIT antagonist may be at least about 1:1.70:0.25, at least about 1:1.70:0.30, at least about 1:1.70:0.35, at least about 1:1.70:0.40, at least about 1:1.70:0.45, at least about 1:1.70:0.50, at least about 1:1.70:0.55, at least about 1:1.70:0.60, at least about 1:1.70:0.65, at least about 1:1.70:0.70, at least about 1:1.70:0.75, at least about 1:1.70:0.80, at least about 1:1.70:0.85, at least about 1:1.70:0.90, at least about 1:1.70:0.95, at least about 1:1.70:1, at least about 1:1.70:1.05, at least about 1:1.70:1.10, at least about 1:1.70:1.15, at least about 1:1.70:1.20, at least about 1:1.70:1.25, at least about 1:1.70:1.30, at least about 1:1.70:1.35, at least about 1:1.70:1.40, at least about 1:1.70:1.45, at least about 1:1.70:1.50, at least about 1:1.70:1.55, at least about 1:1.70:1.60, at least about 1:1.70:1.65, at least about 1:1.70:1.70, at least about 1:1.70:1.75, at least about 1:1.70:1.80, at least about 1:1.70:1.85, at least about 1:1.70:1.90, at least about 1:1.70:1.95, or at least about 1:1.70:2.

In a further embodiment, the ratio of CD96 antagonist to PVRIG antagonist to TIGIT antagonist may be at least about 1:1.75:0.25, at least about 1:1.75:0.30, at least about 1:1.75:0.35, at least about 1:1.75:0.40, at least about 1:1.75:0.45, at least about 1:1.75:0.50, at least about 1:1.75:0.55, at least about 1:1.75:0.60, at least about 1:1.75:0.65, at least about 1:1.75:0.70, at least about 1:1.75:0.75, at least about 1:1.75:0.80, at least about 1:1.75:0.85, at least about 1:1.75:0.90, at least about 1:1.75:0.95, at least about 1:1.75:1, at least about 1:1.75:1.05, at least about 1:1.75:1.10, at least about 1:1.75:1.15, at least about 1:1.75:1.20, at least about 1:1.75:1.25, at least about 1:1.75:1.30, at least about 1:1.75:1.35, at least about 1:1.75:1.40, at least about 1:1.75:1.45, at least about 1:1.75:1.50, at least about 1:1.75:1.55, at least about 1:1.75:1.60, at least about 1:1.75:1.65, at least about 1:1.75:1.70, at least about 1:1.75:1.75, at least about 1:1.75:1.80, at least about 1:1.75:1.85, at least about 1:1.75:1.90, at least about 1:1.75:1.95, or at least about 1:1.75:2.

In a further embodiment, the ratio of CD96 antagonist to PVRIG antagonist to TIGIT antagonist may be at least about 1:1.80:0.25, at least about 1:1.80:0.30, at least about 1:1.80:0.35, at least about 1:1.80:0.40, at least about 1:1.80:0.45, at least about 1:1.80:0.50, at least about 1:1.80:0.55, at least about 1:1.80:0.60, at least about 1:1.80:0.65, at least about 1:1.80:0.70, at least about 1:1.80:0.75, at least about 1:1.80:0.80, at least about 1:1.80:0.85, at least about 1:1.80:0.90, at least about 1:1.80:0.95, at least about 1:1.80:1, at least about 1:1.80:1.05, at least about 1:1.80:1.10, at least about 1:1.80:1.15, at least about 1:1.80:1.20, at least about 1:1.80:1.25, at least about 1:1.80:1.30, at least about 1:1.80:1.35, at least about 1:1.80:1.40, at least about 1:1.80:1.45, at least about 1:1.80:1.50, at least about 1:1.80:1.55, at least about 1:1.80:1.60, at least about 1:1.80:1.65, at least about 1:1.80:1.70, at least about 1:1.80:1.75, at least about 1:1.80:1.80, at least about 1:1.80:1.85, at least about 1:1.80:1.90, at least about 1:1.80:1.95, or at least about 1:1.80:2.

In a further embodiment, the ratio of CD96 antagonist to PVRIG antagonist to TIGIT antagonist may be at least about 1:1.85:0.25, at least about 1:1.85:0.30, at least about 1:1.85:0.35, at least about 1:1.85:0.40, at least about 1:1.85:0.45, at least about 1:1.85:0.50, at least about 1:1.85:0.55, at least about 1:1.85:0.60, at least about 1:1.85:0.65, at least about 1:1.85:0.70, at least about 1:1.85:0.75, at least about 1:1.85:0.80, at least about 1:1.85:0.85, at least about 1:1.85:0.90, at least about 1:1.85:0.95, at least about 1:1.85:1, at least about 1:1.85:1.05, at least about 1:1.85:1.10, at least about 1:1.85:1.15, at least about 1:1.85:1.20, at least about 1:1.85:1.25, at least about 1:1.85:1.30, at least about 1:1.85:1.35, at least about 1:1.85:1.40, at least about 1:1.85:1.45, at least about 1:1.85:1.50, at least about 1:1.85:1.55, at least about 1:1.85:1.60, at least about 1:1.85:1.65, at least about 1:1.85:1.70, at least about 1:1.85:1.75, at least about 1:1.85:1.80, at least about 1:1.85:1.85, at least about 1:1.85:1.90, at least about 1:1.85:1.95, or at least about 1:1.85:2.

In a further embodiment, the ratio of CD96 antagonist to PVRIG antagonist to TIGIT antagonist may be at least about 1:1.90:0.25, at least about 1:1.90:0.30, at least about 1:1.90:0.35, at least about 1:1.90:0.40, at least about 1:1.90:0.45, at least about 1:1.90:0.50, at least about 1:1.90:0.55, at least about 1:1.90:0.60, at least about 1:1.90:0.65, at least about 1:1.90:0.70, at least about 1:1.90:0.75, at least about 1:1.90:0.80, at least about 1:1.90:0.85, at least about 1:1.90:0.90, at least about 1:1.90:0.95, at least about 1:1.90:1, at least about 1:1.90:1.05, at least about 1:1.90:1.10, at least about 1:1.90:1.15, at least about 1:1.90:1.20, at least about 1:1.90:1.25, at least about 1:1.90:1.30, at least about 1:1.90:1.35, at least about 1:1.90:1.40, at least about 1:1.90:1.45, at least about 1:1.90:1.50, at least about 1:1.90:1.55, at least about 1:1.90:1.60, at least about 1:1.90:1.65, at least about 1:1.90:1.70, at least about 1:1.90:1.75, at least about 1:1.90:1.80, at least about 1:1.90:1.85, at least about 1:1.90:1.90, at least about 1:1.90:1.95, or at least about 1:1.90:2.

In a further embodiment, the ratio of CD96 antagonist to PVRIG antagonist to TIGIT antagonist may be at least about 1:1.95:0.25, at least about 1:1.95:0.30, at least about 1:1.95:0.35, at least about 1:1.95:0.40, at least about 1:1.95:0.45, at least about 1:1.95:0.50, at least about 1:1.95:0.55, at least about 1:1.95:0.60, at least about 1:1.95:0.65, at least about 1:1.95:0.70, at least about 1:1.95:0.75, at least about 1:1.95:0.80, at least about 1:1.95:0.85, at least about 1:1.95:0.90, at least about 1:1.95:0.95, at least about 1:1.95:1, at least about 1:1.95:1.05, at least about 1:1.95:1.10, at least about 1:1.95:1.15, at least about 1:1.95:1.20, at least about 1:1.95:1.25, at least about 1:1.95:1.30, at least about 1:1.95:1.35, at least about 1:1.95:1.40, at least about 1:1.95:1.45, at least about 1:1.95:1.50, at least about 1:1.95:1.55, at least about 1:1.95:1.60, at least about 1:1.95:1.65, at least about 1:1.95:1.70, at least about 1:1.95:1.75, at least about 1:1.95:1.80, at least about 1:1.95:1.85, at least about 1:1.95:1.90, at least about 1:1.95:1.95, or at least about 1:1.95:2.

In a further embodiment, the ratio of CD96 antagonist to PVRIG antagonist to TIGIT antagonist may be at least about 1:2:0.25, at least about 1:2:0.30, at least about 1:2:0.35, at least about 1:2:0.40, at least about 1:2:0.45, at least about 1:2:0.50, at least about 1:2:0.55, at least about 1:2:0.60, at least about 1:2:0.65, at least about 1:2:0.70, at least about 1:2:0.75, at least about 1:2:0.80, at least about 1:2:0.85, at least about 1:2:0.90, at least about 1:2:0.95, at least about 1:2:1, at least about 1:2:1.05, at least about 1:2:1.10, at least about 1:2:1.15, at least about 1:2:1.20, at least about 1:2:1.25, at least about 1:2:1.30, at least about 1:2:1.35, at least about 1:2:1.40, at least about 1:2:1.45, at least about 1:2:1.50, at least about 1:2:1.55, at least about 1:2:1.60, at least about 1:2:1.65, at least about 1:2:1.70, at least about 1:2:1.75, at least about 1:2:1.80, at least about 1:2:1.85, at least about 1:2:1.90, at least about 1:2:1.95, or at least about 1:2:2.

In a further embodiment, the ratio of CD96 antagonist to PVRIG antagonist to TIGIT antagonist may be at least about 1:0.25:0.25, at least about 1:0.30:0.25, at least about 1:0.35:0.25, at least about 1:0.40:0.25, at least about 1:0.45:0.25, at least about 1:0.50:0.25, at least about 1:0.55:0.25, at least about 1:0.60:0.25, at least about 1:0.65:0.25, at least about 1:0.70:0.25, at least about 1:0.75:0.25, at least about 1:0.80:0.25, at least about 1:0.85:0.25, at least about 1:0.90:0.25, at least about 1:0.95:0.25, at least about 1:1:0.25, at least about 1:1.05:0.25, at least about 1:1.10:0.25, at least about 1:1.15:0.25, at least about 1:1.20:0.25, at least about 1:1.25:0.25, at least about 1:1.30:0.25, at least about 1:1.35:0.25, at least about 1:1.40:0.25, at least about 1:1.45:0.25, at least about 1:1.50:0.25, at least about 1:1.55:0.25, at least about 1:1.60:0.25, at least about 1:1.65:0.25, at least about 1:1.70:0.25, at least about 1:1.75:0.25, at least about 1:1.80:0.25, at least about 1:1.85:0.25, at least about 1:1.90:0.25, at least about 1:1.95:0.25, or at least about 1:2:0.25.

In a further embodiment, the ratio of CD96 antagonist to PVRIG antagonist to TIGIT antagonist may be at least about 1:0.25:0.30, at least about 1:0.30:0.30, at least about 1:0.35:0.30, at least about 1:0.40:0.30, at least about 1:0.45:0.30, at least about 1:0.50:0.30, at least about 1:0.55:0.30, at least about 1:0.60:0.30, at least about 1:0.65:0.30, at least about 1:0.70:0.30, at least about 1:0.75:0.30, at least about 1:0.80:0.30, at least about 1:0.85:0.30, at least about 1:0.90:0.30, at least about 1:0.95:0.30, at least about 1:1:0.30, at least about 1:1.05:0.30, at least about 1:1.10:0.30, at least about 1:1.15:0.30, at least about 1:1.20:0.30, at least about 1:1.25:0.30, at least about 1:1.30:0.30, at least about 1:1.35:0.30, at least about 1:1.40:0.30, at least about 1:1.45:0.30, at least about 1:1.50:0.30, at least about 1:1.55:0.30, at least about 1:1.60:0.30, at least about 1:1.65:0.30, at least about 1:1.70:0.30, at least about 1:1.75:0.30, at least about 1:1.80:0.30, at least about 1:1.85:0.30, at least about 1:1.90:0.30, at least about 1:1.95:0.30, or at least about 1:2:0.30.

In a further embodiment, the ratio of CD96 antagonist to PVRIG antagonist to TIGIT antagonist may be at least about 1:0.25:0.35, at least about 1:0.30:0.35, at least about 1:0.35:0.35, at least about 1:0.40:0.35, at least about 1:0.45:0.35, at least about 1:0.50:0.35, at least about 1:0.55:0.35, at least about 1:0.60:0.35, at least about 1:0.65:0.35, at least about 1:0.70:0.35, at least about 1:0.75:0.35, at least about 1:0.80:0.35, at least about 1:0.85:0.35, at least about 1:0.90:0.35, at least about 1:0.95:0.35, at least about 1:1:0.35, at least about 1:1.05:0.35, at least about 1:1.10:0.35, at least about 1:1.15:0.35, at least about 1:1.20:0.35, at least about 1:1.25:0.35, at least about 1:1.30:0.35, at least about 1:1.35:0.35, at least about 1:1.40:0.35, at least about 1:1.45:0.35, at least about 1:1.50:0.35, at least about 1:1.55:0.35, at least about 1:1.60:0.35, at least about 1:1.65:0.35, at least about 1:1.70:0.35, at least about 1:1.75:0.35, at least about 1:1.80:0.35, at least about 1:1.85:0.35, at least about 1:1.90:0.35, at least about 1:1.95:0.35, or at least about 1:2:0.35.

In a further embodiment, the ratio of CD96 antagonist to PVRIG antagonist to TIGIT antagonist may be at least about 1:0.25:0.40, at least about 1:0.30:0.40, at least about 1:0.35:0.40, at least about 1:0.40:0.40, at least about 1:0.45:0.40, at least about 1:0.50:0.40, at least about 1:0.55:0.40, at least about 1:0.60:0.40, at least about 1:0.65:0.40, at least about 1:0.70:0.40, at least about 1:0.75:0.40, at least about 1:0.80:0.40, at least about 1:0.85:0.40, at least about 1:0.90:0.40, at least about 1:0.95:0.40, at least about 1:1:0.40, at least about 1:1.05:0.40, at least about 1:1.10:0.40, at least about 1:1.15:0.40, at least about 1:1.20:0.40, at least about 1:1.25:0.40, at least about 1:1.30:0.40, at least about 1:1.35:0.40, at least about 1:1.40:0.40, at least about 1:1.45:0.40, at least about 1:1.50:0.40, at least about 1:1.55:0.40, at least about 1:1.60:0.40, at least about 1:1.65:0.40, at least about 1:1.70:0.40, at least about 1:1.75:0.40, at least about 1:1.80:0.40, at least about 1:1.85:0.40, at least about 1:1.90:0.40, at least about 1:1.95:0.40, or at least about 1:2:0.40.

In a further embodiment, the ratio of CD96 antagonist to PVRIG antagonist to TIGIT antagonist may be at least about 1:0.25:0.45, at least about 1:0.30:0.45, at least about 1:0.35:0.45, at least about 1:0.40:0.45, at least about 1:0.45:0.45, at least about 1:0.50:0.45, at least about 1:0.55:0.45, at least about 1:0.60:0.45, at least about 1:0.65:0.45, at least about 1:0.70:0.45, at least about 1:0.75:0.45, at least about 1:0.80:0.45, at least about 1:0.85:0.45, at least about 1:0.90:0.45, at least about 1:0.95:0.45, at least about 1:1:0.45, at least about 1:1.05:0.45, at least about 1:1.10:0.45, at least about 1:1.15:0.45, at least about 1:1.20:0.45, at least about 1:1.25:0.45, at least about 1:1.30:0.45, at least about 1:1.35:0.45, at least about 1:1.40:0.45, at least about 1:1.45:0.45, at least about 1:1.50:0.45, at least about 1:1.55:0.45, at least about 1:1.60:0.45, at least about 1:1.65:0.45, at least about 1:1.70:0.45, at least about 1:1.75:0.45, at least about 1:1.80:0.45, at least about 1:1.85:0.45, at least about 1:1.90:0.45, at least about 1:1.95:0.45, or at least about 1:2:0.45.

In a further embodiment, the ratio of CD96 antagonist to PVRIG antagonist to TIGIT antagonist may be at least about 1:0.25:0.50, at least about 1:0.30:0.50, at least about 1:0.35:0.50, at least about 1:0.40:0.50, at least about 1:0.45:0.50, at least about 1:0.50:0.50, at least about 1:0.55:0.50, at least about 1:0.60:0.50, at least about 1:0.65:0.50, at least about 1:0.70:0.50, at least about 1:0.75:0.50, at least about 1:0.80:0.50, at least about 1:0.85:0.50, at least about 1:0.90:0.50, at least about 1:0.95:0.50, at least about 1:1:0.50, at least about 1:1.05:0.50, at least about 1:1.10:0.50, at least about 1:1.15:0.50, at least about 1:1.20:0.50, at least about 1:1.25:0.50, at least about 1:1.30:0.50, at least about 1:1.35:0.50, at least about 1:1.40:0.50, at least about 1:1.45:0.50, at least about 1:1.50:0.50, at least about 1:1.55:0.50, at least about 1:1.60:0.50, at least about 1:1.65:0.50, at least about 1:1.70:0.50, at least about 1:1.75:0.50, at least about 1:1.80:0.50, at least about 1:1.85:0.50, at least about 1:1.90:0.50, at least about 1:1.95:0.50, or at least about 1:2:0.50.

In a further embodiment, the ratio of CD96 antagonist to PVRIG antagonist to TIGIT antagonist may be at least about 1:0.25:0.55, at least about 1:0.30:0.55, at least about 1:0.35:0.55, at least about 1:0.40:0.55, at least about 1:0.45:0.55, at least about 1:0.50:0.55, at least about 1:0.55:0.55, at least about 1:0.60:0.55, at least about 1:0.65:0.55, at least about 1:0.70:0.55, at least about 1:0.75:0.55, at least about 1:0.80:0.55, at least about 1:0.85:0.55, at least about 1:0.90:0.55, at least about 1:0.95:0.55, at least about 1:1:0.55, at least about 1:1.05:0.55, at least about 1:1.10:0.55, at least about 1:1.15:0.55, at least about 1:1.20:0.55, at least about 1:1.25:0.55, at least about 1:1.30:0.55, at least about 1:1.35:0.55, at least about 1:1.40:0.55, at least about 1:1.45:0.55, at least about 1:1.50:0.55, at least about 1:1.55:0.55, at least about 1:1.60:0.55, at least about 1:1.65:0.55, at least about 1:1.70:0.55, at least about 1:1.75:0.55, at least about 1:1.80:0.55, at least about 1:1.85:0.55, at least about 1:1.90:0.55, at least about 1:1.95:0.55, or at least about 1:2:0.55.

In a further embodiment, the ratio of CD96 antagonist to PVRIG antagonist to TIGIT antagonist may be at least about 1:0.25:0.60, at least about 1:0.30:0.60, at least about 1:0.35:0.60, at least about 1:0.40:0.60, at least about 1:0.45:0.60, at least about 1:0.50:0.60, at least about 1:0.55:0.60, at least about 1:0.60:0.60, at least about 1:0.65:0.60, at least about 1:0.70:0.60, at least about 1:0.75:0.60, at least about 1:0.80:0.60, at least about 1:0.85:0.60, at least about 1:0.90:0.60, at least about 1:0.95:0.60, at least about 1:1:0.60, at least about 1:1.05:0.60, at least about 1:1.10:0.60, at least about 1:1.15:0.60, at least about 1:1.20:0.60, at least about 1:1.25:0.60, at least about 1:1.30:0.60, at least about 1:1.35:0.60, at least about 1:1.40:0.60, at least about 1:1.45:0.60, at least about 1:1.50:0.60, at least about 1:1.55:0.60, at least about 1:1.60:0.60, at least about 1:1.65:0.60, at least about 1:1.70:0.60, at least about 1:1.75:0.60, at least about 1:1.80:0.60, at least about 1:1.85:0.60, at least about 1:1.90:0.60, at least about 1:1.95:0.60, or at least about 1:2:0.60.

In a further embodiment, the ratio of CD96 antagonist to PVRIG antagonist to TIGIT antagonist may be at least about 1:0.25:0.65, at least about 1:0.30:0.65, at least about 1:0.35:0.65, at least about 1:0.40:0.65, at least about 1:0.45:0.65, at least about 1:0.50:0.65, at least about 1:0.55:0.65, at least about 1:0.60:0.65, at least about 1:0.65:0.65, at least about 1:0.70:0.65, at least about 1:0.75:0.65, at least about 1:0.80:0.65, at least about 1:0.85:0.65, at least about 1:0.90:0.65, at least about 1:0.95:0.65, at least about 1:1:0.65, at least about 1:1.05:0.65, at least about 1:1.10:0.65, at least about 1:1.15:0.65, at least about 1:1.20:0.65, at least about 1:1.25:0.65, at least about 1:1.30:0.65, at least about 1:1.35:0.65, at least about 1:1.40:0.65, at least about 1:1.45:0.65, at least about 1:1.50:0.65, at least about 1:1.55:0.65, at least about 1:1.60:0.65, at least about 1:1.65:0.65, at least about 1:1.70:0.65, at least about 1:1.75:0.65, at least about 1:1.80:0.65, at least about 1:1.85:0.65, at least about 1:1.90:0.65, at least about 1:1.95:0.65, or at least about 1:2:0.65.

In a further embodiment, the ratio of CD96 antagonist to PVRIG antagonist to TIGIT antagonist may be at least about 1:0.25:0.70, at least about 1:0.30:0.70, at least about 1:0.35:0.70, at least about 1:0.40:0.70, at least about 1:0.45:0.70, at least about 1:0.50:0.70, at least about 1:0.55:0.70, at least about 1:0.60:0.70, at least about 1:0.65:0.70, at least about 1:0.70:0.70, at least about 1:0.75:0.70, at least about 1:0.80:0.70, at least about 1:0.85:0.70, at least about 1:0.90:0.70, at least about 1:0.95:0.70, at least about 1:1:0.70, at least about 1:1.05:0.70, at least about 1:1.10:0.70, at least about 1:1.15:0.70, at least about 1:1.20:0.70, at least about 1:1.25:0.70, at least about 1:1.30:0.70, at least about 1:1.35:0.70, at least about 1:1.40:0.70, at least about 1:1.45:0.70, at least about 1:1.50:0.70, at least about 1:1.55:0.70, at least about 1:1.60:0.70, at least about 1:1.65:0.70, at least about 1:1.70:0.70, at least about 1:1.75:0.70, at least about 1:1.80:0.70, at least about 1:1.85:0.70, at least about 1:1.90:0.70, at least about 1:1.95:0.70, or at least about 1:2:0.70.

In a further embodiment, the ratio of CD96 antagonist to PVRIG antagonist to TIGIT antagonist may be at least about 1:0.25:0.75, at least about 1:0.30:0.75, at least about 1:0.35:0.75, at least about 1:0.40:0.75, at least about 1:0.45:0.75, at least about 1:0.50:0.75, at least about 1:0.55:0.75, at least about 1:0.60:0.75, at least about 1:0.65:0.75, at least about 1:0.70:0.75, at least about 1:0.75:0.75, at least about 1:0.80:0.75, at least about 1:0.85:0.75, at least about 1:0.90:0.75, at least about 1:0.95:0.75, at least about 1:1:0.75, at least about 1:1.05:0.75, at least about 1:1.10:0.75, at least about 1:1.15:0.75, at least about 1:1.20:0.75, at least about 1:1.25:0.75, at least about 1:1.30:0.75, at least about 1:1.35:0.75, at least about 1:1.40:0.75, at least about 1:1.45:0.75, at least about 1:1.50:0.75, at least about 1:1.55:0.75, at least about 1:1.60:0.75, at least about 1:1.65:0.75, at least about 1:1.70:0.75, at least about 1:1.75:0.75, at least about 1:1.80:0.75, at least about 1:1.85:0.75, at least about 1:1.90:0.75, at least about 1:1.95:0.75, or at least about 1:2:0.75.

In a further embodiment, the ratio of CD96 antagonist to PVRIG antagonist to TIGIT antagonist may be at least about 1:0.25:0.80, at least about 1:0.30:0.80, at least about 1:0.35:0.80, at least about 1:0.40:0.80, at least about 1:0.45:0.80, at least about 1:0.50:0.80, at least about 1:0.55:0.80, at least about 1:0.60:0.80, at least about 1:0.65:0.80, at least about 1:0.70:0.80, at least about 1:0.75:0.80, at least about 1:0.80:0.80, at least about 1:0.85:0.80, at least about 1:0.90:0.80, at least about 1:0.95:0.80, at least about 1:1:0.80, at least about 1:1.05:0.80, at least about 1:1.10:0.80, at least about 1:1.15:0.80, at least about 1:1.20:0.80, at least about 1:1.25:0.80, at least about 1:1.30:0.80, at least about 1:1.35:0.80, at least about 1:1.40:0.80, at least about 1:1.45:0.80, at least about 1:1.50:0.80, at least about 1:1.55:0.80, at least about 1:1.60:0.80, at least about 1:1.65:0.80, at least about 1:1.70:0.80, at least about 1:1.75:0.80, at least about 1:1.80:0.80, at least about 1:1.85:0.80, at least about 1:1.90:0.80, at least about 1:1.95:0.80, or at least about 1:2:0.80.

In a further embodiment, the ratio of CD96 antagonist to PVRIG antagonist to TIGIT antagonist may be at least about 1:0.25:0.85, at least about 1:0.30:0.85, at least about 1:0.35:0.85, at least about 1:0.40:0.85, at least about 1:0.45:0.85, at least about 1:0.50:0.85, at least about 1:0.55:0.85, at least about 1:0.60:0.85, at least about 1:0.65:0.85, at least about 1:0.70:0.85, at least about 1:0.75:0.85, at least about 1:0.80:0.85, at least about 1:0.85:0.85, at least about 1:0.90:0.85, at least about 1:0.95:0.85, at least about 1:1:0.85, at least about 1:1.05:0.85, at least about 1:1.10:0.85, at least about 1:1.15:0.85, at least about 1:1.20:0.85, at least about 1:1.25:0.85, at least about 1:1.30:0.85, at least about 1:1.35:0.85, at least about 1:1.40:0.85, at least about 1:1.45:0.85, at least about 1:1.50:0.85, at least about 1:1.55:0.85, at least about 1:1.60:0.85, at least about 1:1.65:0.85, at least about 1:1.70:0.85, at least about 1:1.75:0.85, at least about 1:1.80:0.85, at least about 1:1.85:0.85, at least about 1:1.90:0.85, at least about 1:1.95:0.85, or at least about 1:2:0.85.

In a further embodiment, the ratio of CD96 antagonist to PVRIG antagonist to TIGIT antagonist may be at least about 1:0.25:0.90, at least about 1:0.30:0.90, at least about 1:0.35:0.90, at least about 1:0.40:0.90, at least about 1:0.45:0.90, at least about 1:0.50:0.90, at least about 1:0.55:0.90, at least about 1:0.60:0.90, at least about 1:0.65:0.90, at least about 1:0.70:0.90, at least about 1:0.75:0.90, at least about 1:0.80:0.90, at least about 1:0.85:0.90, at least about 1:0.90:0.90, at least about 1:0.95:0.90, at least about 1:1:0.90, at least about 1:1.05:0.90, at least about 1:1.10:0.90, at least about 1:1.15:0.90, at least about 1:1.20:0.90, at least about 1:1.25:0.90, at least about 1:1.30:0.90, at least about 1:1.35:0.90, at least about 1:1.40:0.90, at least about 1:1.45:0.90, at least about 1:1.50:0.90, at least about 1:1.55:0.90, at least about 1:1.60:0.90, at least about 1:1.65:0.90, at least about 1:1.70:0.90, at least about 1:1.75:0.90, at least about 1:1.80:0.90, at least about 1:1.85:0.90, at least about 1:1.90:0.90, at least about 1:1.95:0.90, or at least about 1:2:0.90.

In a further embodiment, the ratio of CD96 antagonist to PVRIG antagonist to TIGIT antagonist may be at least about 1:0.25:0.95, at least about 1:0.30:0.95, at least about 1:0.35:0.95, at least about 1:0.40:0.95, at least about 1:0.45:0.95, at least about 1:0.50:0.95, at least about 1:0.55:0.95, at least about 1:0.60:0.95, at least about 1:0.65:0.95, at least about 1:0.70:0.95, at least about 1:0.75:0.95, at least about 1:0.80:0.95, at least about 1:0.85:0.95, at least about 1:0.90:0.95, at least about 1:0.95:0.95, at least about 1:1:0.95, at least about 1:1.05:0.95, at least about 1:1.10:0.95, at least about 1:1.15:0.95, at least about 1:1.20:0.95, at least about 1:1.25:0.95, at least about 1:1.30:0.95, at least about 1:1.35:0.95, at least about 1:1.40:0.95, at least about 1:1.45:0.95, at least about 1:1.50:0.95, at least about 1:1.55:0.95, at least about 1:1.60:0.95, at least about 1:1.65:0.95, at least about 1:1.70:0.95, at least about 1:1.75:0.95, at least about 1:1.80:0.95, at least about 1:1.85:0.95, at least about 1:1.90:0.95, at least about 1:1.95:0.95, or at least about 1:2:0.95.

In a further embodiment, the ratio of CD96 antagonist to PVRIG antagonist to TIGIT antagonist may be at least about 1:0.25:1, at least about 1:0.30:1, at least about 1:0.35:1, at least about 1:0.40:1, at least about 1:0.45:1, at least about 1:0.50:1, at least about 1:0.55:1, at least about 1:0.60:1, at least about 1:0.65:1, at least about 1:0.70:1, at least about 1:0.75:1, at least about 1:0.80:1, at least about 1:0.85:1, at least about 1:0.90:1, at least about 1:0.95:1, at least about 1:1:1, at least about 1:1.05:1, at least about 1:1.10:1, at least about 1:1.15:1, at least about 1:1.20:1, at least about 1:1.25:1, at least about 1:1.30:1, at least about 1:1.35:1, at least about 1:1.40:1, at least about 1:1.45:1, at least about 1:1.50:1, at least about 1:1.55:1, at least about 1:1.60:1, at least about 1:1.65:1, at least about 1:1.70:1, at least about 1:1.75:1, at least about 1:1.80:1, at least about 1:1.85:1, at least about 1:1.90:1, at least about 1:1.95:1, or at least about 1:2:1.

In a further embodiment, the ratio of CD96 antagonist to PVRIG antagonist to TIGIT antagonist may be at least about 1:0.25:1.05, at least about 1:0.30:1.05, at least about 1:0.35:1.05, at least about 1:0.40:1.05, at least about 1:0.45:1.05, at least about 1:0.50:1.05, at least about 1:0.55:1.05, at least about 1:0.60:1.05, at least about 1:0.65:1.05, at least about 1:0.70:1.05, at least about 1:0.75:1.05, at least about 1:0.80:1.05, at least about 1:0.85:1.05, at least about 1:0.90:1.05, at least about 1:0.95:1.05, at least about 1:1:1.05, at least about 1:1.05:1.05, at least about 1:1.10:1.05, at least about 1:1.15:1.05, at least about 1:1.20:1.05, at least about 1:1.25:1.05, at least about 1:1.30:1.05, at least about 1:1.35:1.05, at least about 1:1.40:1.05, at least about 1:1.45:1.05, at least about 1:1.50:1.05, at least about 1:1.55:1.05, at least about 1:1.60:1.05, at least about 1:1.65:1.05, at least about 1:1.70:1.05, at least about 1:1.75:1.05, at least about 1:1.80:1.05, at least about 1:1.85:1.05, at least about 1:1.90:1.05, at least about 1:1.95:1.05, or at least about 1:2:1.05.

In a further embodiment, the ratio of CD96 antagonist to PVRIG antagonist to TIGIT antagonist may be at least about 1:0.25:1.10, at least about 1:0.30:1.10, at least about 1:0.35:1.10, at least about 1:0.40:1.10, at least about 1:0.45:1.10, at least about 1:0.50:1.10, at least about 1:0.55:1.10, at least about 1:0.60:1.10, at least about 1:0.65:1.10, at least about 1:0.70:1.10, at least about 1:0.75:1.10, at least about 1:0.80:1.10, at least about 1:0.85:1.10, at least about 1:0.90:1.10, at least about 1:0.95:1.10, at least about 1:1:1.10, at least about 1:1.05:1.10, at least about 1:1.10:1.10, at least about 1:1.15:1.10, at least about 1:1.20:1.10, at least about 1:1.25:1.10, at least about 1:1.30:1.10, at least about 1:1.35:1.10, at least about 1:1.40:1.10, at least about 1:1.45:1.10, at least about 1:1.50:1.10, at least about 1:1.55:1.10, at least about 1:1.60:1.10, at least about 1:1.65:1.10, at least about 1:1.70:1.10, at least about 1:1.75:1.10, at least about 1:1.80:1.10, at least about 1:1.85:1.10, at least about 1:1.90:1.10, at least about 1:1.95:1.10, or at least about 1:2:1.10.

In a further embodiment, the ratio of CD96 antagonist to PVRIG antagonist to TIGIT antagonist may be at least about 1:0.25:1.15, at least about 1:0.30:1.15, at least about 1:0.35:1.15, at least about 1:0.40:1.15, at least about 1:0.45:1.15, at least about 1:0.50:1.15, at least about 1:0.55:1.15, at least about 1:0.60:1.15, at least about 1:0.65:1.15, at least about 1:0.70:1.15, at least about 1:0.75:1.15, at least about 1:0.80:1.15, at least about 1:0.85:1.15, at least about 1:0.90:1.15, at least about 1:0.95:1.15, at least about 1:1:1.15, at least about 1:1.05:1.15, at least about 1:1.10:1.15, at least about 1:1.15:1.15, at least about 1:1.20:1.15, at least about 1:1.25:1.15, at least about 1:1.30:1.15, at least about 1:1.35:1.15, at least about 1:1.40:1.15, at least about 1:1.45:1.15, at least about 1:1.50:1.15, at least about 1:1.55:1.15, at least about 1:1.60:1.15, at least about 1:1.65:1.15, at least about 1:1.70:1.15, at least about 1:1.75:1.15, at least about 1:1.80:1.15, at least about 1:1.85:1.15, at least about 1:1.90:1.15, at least about 1:1.95:1.15, or at least about 1:2:1.15.

In a further embodiment, the ratio of CD96 antagonist to PVRIG antagonist to TIGIT antagonist may be at least about 1:0.25:1.20, at least about 1:0.30:1.20, at least about 1:0.35:1.20, at least about 1:0.40:1.20, at least about 1:0.45:1.20, at least about 1:0.50:1.20, at least about 1:0.55:1.20, at least about 1:0.60:1.20, at least about 1:0.65:1.20, at least about 1:0.70:1.20, at least about 1:0.75:1.20, at least about 1:0.80:1.20, at least about 1:0.85:1.20, at least about 1:0.90:1.20, at least about 1:0.95:1.20, at least about 1:1:1.20, at least about 1:1.05:1.20, at least about 1:1.10:1.20, at least about 1:1.15:1.20, at least about 1:1.20:1.20, at least about 1:1.25:1.20, at least about 1:1.30:1.20, at least about 1:1.35:1.20, at least about 1:1.40:1.20, at least about 1:1.45:1.20, at least about 1:1.50:1.20, at least about 1:1.55:1.20, at least about 1:1.60:1.20, at least about 1:1.65:1.20, at least about 1:1.70:1.20, at least about 1:1.75:1.20, at least about 1:1.80:1.20, at least about 1:1.85:1.20, at least about 1:1.90:1.20, at least about 1:1.95:1.20, or at least about 1:2:1.20.

In a further embodiment, the ratio of CD96 antagonist to PVRIG antagonist to TIGIT antagonist may be at least about 1:0.25:1.25, at least about 1:0.30:1.25, at least about 1:0.35:1.25, at least about 1:0.40:1.25, at least about 1:0.45:1.25, at least about 1:0.50:1.25, at least about 1:0.55:1.25, at least about 1:0.60:1.25, at least about 1:0.65:1.25, at least about 1:0.70:1.25, at least about 1:0.75:1.25, at least about 1:0.80:1.25, at least about 1:0.85:1.25, at least about 1:0.90:1.25, at least about 1:0.95:1.25, at least about 1:1:1.25, at least about 1:1.05:1.25, at least about 1:1.10:1.25, at least about 1:1.15:1.25, at least about 1:1.20:1.25, at least about 1:1.25:1.25, at least about 1:1.30:1.25, at least about 1:1.35:1.25, at least about 1:1.40:1.25, at least about 1:1.45:1.25, at least about 1:1.50:1.25, at least about 1:1.55:1.25, at least about 1:1.60:1.25, at least about 1:1.65:1.25, at least about 1:1.70:1.25, at least about 1:1.75:1.25, at least about 1:1.80:1.25, at least about 1:1.85:1.25, at least about 1:1.90:1.25, at least about 1:1.95:1.25, or at least about 1:2:1.25.

In a further embodiment, the ratio of CD96 antagonist to PVRIG antagonist to TIGIT antagonist may be at least about 1:0.25:1.30, at least about 1:0.30:1.30, at least about 1:0.35:1.30, at least about 1:0.40:1.30, at least about 1:0.45:1.30, at least about 1:0.50:1.30, at least about 1:0.55:1.30, at least about 1:0.60:1.30, at least about 1:0.65:1.30, at least about 1:0.70:1.30, at least about 1:0.75:1.30, at least about 1:0.80:1.30, at least about 1:0.85:1.30, at least about 1:0.90:1.30, at least about 1:0.95:1.30, at least about 1:1:1.30, at least about 1:1.05:1.30, at least about 1:1.10:1.30, at least about 1:1.15:1.30, at least about 1:1.20:1.30, at least about 1:1.25:1.30, at least about 1:1.30:1.30, at least about 1:1.35:1.30, at least about 1:1.40:1.30, at least about 1:1.45:1.30, at least about 1:1.50:1.30, at least about 1:1.55:1.30, at least about 1:1.60:1.30, at least about 1:1.65:1.30, at least about 1:1.70:1.30, at least about 1:1.75:1.30, at least about 1:1.80:1.30, at least about 1:1.85:1.30, at least about 1:1.90:1.30, at least about 1:1.95:1.30, or at least about 1:2:1.30.

In a further embodiment, the ratio of CD96 antagonist to PVRIG antagonist to TIGIT antagonist may be at least about 1:0.25:1.35, at least about 1:0.30:1.35, at least about 1:0.35:1.35, at least about 1:0.40:1.35, at least about 1:0.45:1.35, at least about 1:0.50:1.35, at least about 1:0.55:1.35, at least about 1:0.60:1.35, at least about 1:0.65:1.35, at least about 1:0.70:1.35, at least about 1:0.75:1.35, at least about 1:0.80:1.35, at least about 1:0.85:1.35, at least about 1:0.90:1.35, at least about 1:0.95:1.35, at least about 1:1:1.35, at least about 1:1.05:1.35, at least about 1:1.10:1.35, at least about 1:1.15:1.35, at least about 1:1.20:1.35, at least about 1:1.25:1.35, at least about 1:1.30:1.35, at least about 1:1.35:1.35, at least about 1:1.40:1.35, at least about 1:1.45:1.35, at least about 1:1.50:1.35, at least about 1:1.55:1.35, at least about 1:1.60:1.35, at least about 1:1.65:1.35, at least about 1:1.70:1.35, at least about 1:1.75:1.35, at least about 1:1.80:1.35, at least about 1:1.85:1.35, at least about 1:1.90:1.35, at least about 1:1.95:1.35, or at least about 1:2:1.35.

In a further embodiment, the ratio of CD96 antagonist to PVRIG antagonist to TIGIT antagonist may be at least about 1:0.25:1.40, at least about 1:0.30:1.40, at least about 1:0.35:1.40, at least about 1:0.40:1.40, at least about 1:0.45:1.40, at least about 1:0.50:1.40, at least about 1:0.55:1.40, at least about 1:0.60:1.40, at least about 1:0.65:1.40, at least about 1:0.70:1.40, at least about 1:0.75:1.40, at least about 1:0.80:1.40, at least about 1:0.85:1.40, at least about 1:0.90:1.40, at least about 1:0.95:1.40, at least about 1:1:1.40, at least about 1:1.05:1.40, at least about 1:1.10:1.40, at least about 1:1.15:1.40, at least about 1:1.20:1.40, at least about 1:1.25:1.40, at least about 1:1.30:1.40, at least about 1:1.35:1.40, at least about 1:1.40:1.40, at least about 1:1.45:1.40, at least about 1:1.50:1.40, at least about 1:1.55:1.40, at least about 1:1.60:1.40, at least about 1:1.65:1.40, at least about 1:1.70:1.40, at least about 1:1.75:1.40, at least about 1:1.80:1.40, at least about 1:1.85:1.40, at least about 1:1.90:1.40, at least about 1:1.95:1.40, or at least about 1:2:1.40.

In a further embodiment, the ratio of CD96 antagonist to PVRIG antagonist to TIGIT antagonist may be at least about 1:0.25:1.45, at least about 1:0.30:1.45, at least about 1:0.35:1.45, at least about 1:0.40:1.45, at least about 1:0.45:1.45, at least about 1:0.50:1.45, at least about 1:0.55:1.45, at least about 1:0.60:1.45, at least about 1:0.65:1.45, at least about 1:0.70:1.45, at least about 1:0.75:1.45, at least about 1:0.80:1.45, at least about 1:0.85:1.45, at least about 1:0.90:1.45, at least about 1:0.95:1.45, at least about 1:1:1.45, at least about 1:1.05:1.45, at least about 1:1.10:1.45, at least about 1:1.15:1.45, at least about 1:1.20:1.45, at least about 1:1.25:1.45, at least about 1:1.30:1.45, at least about 1:1.35:1.45, at least about 1:1.40:1.45, at least about 1:1.45:1.45, at least about 1:1.50:1.45, at least about 1:1.55:1.45, at least about 1:1.60:1.45, at least about 1:1.65:1.45, at least about 1:1.70:1.45, at least about 1:1.75:1.45, at least about 1:1.80:1.45, at least about 1:1.85:1.45, at least about 1:1.90:1.45, at least about 1:1.95:1.45, or at least about 1:2:1.45.

In a further embodiment, the ratio of CD96 antagonist to PVRIG antagonist to TIGIT antagonist may be at least about 1:0.25:1.50, at least about 1:0.30:1.50, at least about 1:0.35:1.50, at least about 1:0.40:1.50, at least about 1:0.45:1.50, at least about 1:0.50:1.50, at least about 1:0.55:1.50, at least about 1:0.60:1.50, at least about 1:0.65:1.50, at least about 1:0.70:1.50, at least about 1:0.75:1.50, at least about 1:0.80:1.50, at least about 1:0.85:1.50, at least about 1:0.90:1.50, at least about 1:0.95:1.50, at least about 1:1:1.50, at least about 1:1.05:1.50, at least about 1:1.10:1.50, at least about 1:1.15:1.50, at least about 1:1.20:1.50, at least about 1:1.25:1.50, at least about 1:1.30:1.50, at least about 1:1.35:1.50, at least about 1:1.40:1.50, at least about 1:1.45:1.50, at least about 1:1.50:1.50, at least about 1:1.55:1.50, at least about 1:1.60:1.50, at least about 1:1.65:1.50, at least about 1:1.70:1.50, at least about 1:1.75:1.50, at least about 1:1.80:1.50, at least about 1:1.85:1.50, at least about 1:1.90:1.50, at least about 1:1.95:1.50, or at least about 1:2:1.50.

In a further embodiment, the ratio of CD96 antagonist to PVRIG antagonist to TIGIT antagonist may be at least about 1:0.25:1.55, at least about 1:0.30:1.55, at least about 1:0.35:1.55, at least about 1:0.40:1.55, at least about 1:0.45:1.55, at least about 1:0.50:1.55, at least about 1:0.55:1.55, at least about 1:0.60:1.55, at least about 1:0.65:1.55, at least about 1:0.70:1.55, at least about 1:0.75:1.55, at least about 1:0.80:1.55, at least about 1:0.85:1.55, at least about 1:0.90:1.55, at least about 1:0.95:1.55, at least about 1:1:1.55, at least about 1:1.05:1.55, at least about 1:1.10:1.55, at least about 1:1.15:1.55, at least about 1:1.20:1.55, at least about 1:1.25:1.55, at least about 1:1.30:1.55, at least about 1:1.35:1.55, at least about 1:1.40:1.55, at least about 1:1.45:1.55, at least about 1:1.50:1.55, at least about 1:1.55:1.55, at least about 1:1.60:1.55, at least about 1:1.65:1.55, at least about 1:1.70:1.55, at least about 1:1.75:1.55, at least about 1:1.80:1.55, at least about 1:1.85:1.55, at least about 1:1.90:1.55, at least about 1:1.95:1.55, or at least about 1:2:1.55.

In a further embodiment, the ratio of CD96 antagonist to PVRIG antagonist to TIGIT antagonist may be at least about 1:0.25:1.60, at least about 1:0.30:1.60, at least about 1:0.35:1.60, at least about 1:0.40:1.60, at least about 1:0.45:1.60, at least about 1:0.50:1.60, at least about 1:0.55:1.60, at least about 1:0.60:1.60, at least about 1:0.65:1.60, at least about 1:0.70:1.60, at least about 1:0.75:1.60, at least about 1:0.80:1.60, at least about 1:0.85:1.60, at least about 1:0.90:1.60, at least about 1:0.95:1.60, at least about 1:1:1.60, at least about 1:1.05:1.60, at least about 1:1.10:1.60, at least about 1:1.15:1.60, at least about 1:1.20:1.60, at least about 1:1.25:1.60, at least about 1:1.30:1.60, at least about 1:1.35:1.60, at least about 1:1.40:1.60, at least about 1:1.45:1.60, at least about 1:1.50:1.60, at least about 1:1.55:1.60, at least about 1:1.60:1.60, at least about 1:1.65:1.60, at least about 1:1.70:1.60, at least about 1:1.75:1.60, at least about 1:1.80:1.60, at least about 1:1.85:1.60, at least about 1:1.90:1.60, at least about 1:1.95:1.60, or at least about 1:2:1.60.

In a further embodiment, the ratio of CD96 antagonist to PVRIG antagonist to TIGIT antagonist may be at least about 1:0.25:1.65, at least about 1:0.30:1.65, at least about 1:0.35:1.65, at least about 1:0.40:1.65, at least about 1:0.45:1.65, at least about 1:0.50:1.65, at least about 1:0.55:1.65, at least about 1:0.60:1.65, at least about 1:0.65:1.65, at least about 1:0.70:1.65, at least about 1:0.75:1.65, at least about 1:0.80:1.65, at least about 1:0.85:1.65, at least about 1:0.90:1.65, at least about 1:0.95:1.65, at least about 1:1:1.65, at least about 1:1.05:1.65, at least about 1:1.10:1.65, at least about 1:1.15:1.65, at least about 1:1.20:1.65, at least about 1:1.25:1.65, at least about 1:1.30:1.65, at least about 1:1.35:1.65, at least about 1:1.40:1.65, at least about 1:1.45:1.65, at least about 1:1.50:1.65, at least about 1:1.55:1.65, at least about 1:1.60:1.65, at least about 1:1.65:1.65, at least about 1:1.70:1.65, at least about 1:1.75:1.65, at least about 1:1.80:1.65, at least about 1:1.85:1.65, at least about 1:1.90:1.65, at least about 1:1.95:1.65, or at least about 1:2:1.65.

In a further embodiment, the ratio of CD96 antagonist to PVRIG antagonist to TIGIT antagonist may be at least about 1:0.25:1.70, at least about 1:0.30:1.70, at least about 1:0.35:1.70, at least about 1:0.40:1.70, at least about 1:0.45:1.70, at least about 1:0.50:1.70, at least about 1:0.55:1.70, at least about 1:0.60:1.70, at least about 1:0.65:1.70, at least about 1:0.70:1.70, at least about 1:0.75:1.70, at least about 1:0.80:1.70, at least about 1:0.85:1.70, at least about 1:0.90:1.70, at least about 1:0.95:1.70, at least about 1:1:1.70, at least about 1:1.05:1.70, at least about 1:1.10:1.70, at least about 1:1.15:1.70, at least about 1:1.20:1.70, at least about 1:1.25:1.70, at least about 1:1.30:1.70, at least about 1:1.35:1.70, at least about 1:1.40:1.70, at least about 1:1.45:1.70, at least about 1:1.50:1.70, at least about 1:1.55:1.70, at least about 1:1.60:1.70, at least about 1:1.65:1.70, at least about 1:1.70:1.70, at least about 1:1.75:1.70, at least about 1:1.80:1.70, at least about 1:1.85:1.70, at least about 1:1.90:1.70, at least about 1:1.95:1.70, or at least about 1:2:1.70.

In a further embodiment, the ratio of CD96 antagonist to PVRIG antagonist to TIGIT antagonist may be at least about 1:0.25:1.75, at least about 1:0.30:1.75, at least about 1:0.35:1.75, at least about 1:0.40:1.75, at least about 1:0.45:1.75, at least about 1:0.50:1.75, at least about 1:0.55:1.75, at least about 1:0.60:1.75, at least about 1:0.65:1.75, at least about 1:0.70:1.75, at least about 1:0.75:1.75, at least about 1:0.80:1.75, at least about 1:0.85:1.75, at least about 1:0.90:1.75, at least about 1:0.95:1.75, at least about 1:1:1.75, at least about 1:1.05:1.75, at least about 1:1.10:1.75, at least about 1:1.15:1.75, at least about 1:1.20:1.75, at least about 1:1.25:1.75, at least about 1:1.30:1.75, at least about 1:1.35:1.75, at least about 1:1.40:1.75, at least about 1:1.45:1.75, at least about 1:1.50:1.75, at least about 1:1.55:1.75, at least about 1:1.60:1.75, at least about 1:1.65:1.75, at least about 1:1.70:1.75, at least about 1:1.75:1.75, at least about 1:1.80:1.75, at least about 1:1.85:1.75, at least about 1:1.90:1.75, at least about 1:1.95:1.75, or at least about 1:2:1.75.

In a further embodiment, the ratio of CD96 antagonist to PVRIG antagonist to TIGIT antagonist may be at least about 1:0.25:1.80, at least about 1:0.30:1.80, at least about 1:0.35:1.80, at least about 1:0.40:1.80, at least about 1:0.45:1.80, at least about 1:0.50:1.80, at least about 1:0.55:1.80, at least about 1:0.60:1.80, at least about 1:0.65:1.80, at least about 1:0.70:1.80, at least about 1:0.75:1.80, at least about 1:0.80:1.80, at least about 1:0.85:1.80, at least about 1:0.90:1.80, at least about 1:0.95:1.80, at least about 1:1:1.80, at least about 1:1.05:1.80, at least about 1:1.10:1.80, at least about 1:1.15:1.80, at least about 1:1.20:1.80, at least about 1:1.25:1.80, at least about 1:1.30:1.80, at least about 1:1.35:1.80, at least about 1:1.40:1.80, at least about 1:1.45:1.80, at least about 1:1.50:1.80, at least about 1:1.55:1.80, at least about 1:1.60:1.80, at least about 1:1.65:1.80, at least about 1:1.70:1.80, at least about 1:1.75:1.80, at least about 1:1.80:1.80, at least about 1:1.85:1.80, at least about 1:1.90:1.80, at least about 1:1.95:1.80, or at least about 1:2:1.80.

In a further embodiment, the ratio of CD96 antagonist to PVRIG antagonist to TIGIT antagonist may be at least about 1:0.25:1.85, at least about 1:0.30:1.85, at least about 1:0.35:1.85, at least about 1:0.40:1.85, at least about 1:0.45:1.85, at least about 1:0.50:1.85, at least about 1:0.55:1.85, at least about 1:0.60:1.85, at least about 1:0.65:1.85, at least about 1:0.70:1.85, at least about 1:0.75:1.85, at least about 1:0.80:1.85, at least about 1:0.85:1.85, at least about 1:0.90:1.85, at least about 1:0.95:1.85, at least about 1:1:1.85, at least about 1:1.05:1.85, at least about 1:1.10:1.85, at least about 1:1.15:1.85, at least about 1:1.20:1.85, at least about 1:1.25:1.85, at least about 1:1.30:1.85, at least about 1:1.35:1.85, at least about 1:1.40:1.85, at least about 1:1.45:1.85, at least about 1:1.50:1.85, at least about 1:1.55:1.85, at least about 1:1.60:1.85, at least about 1:1.65:1.85, at least about 1:1.70:1.85, at least about 1:1.75:1.85, at least about 1:1.80:1.85, at least about 1:1.85:1.85, at least about 1:1.90:1.85, at least about 1:1.95:1.85, or at least about 1:2:1.85.

In a further embodiment, the ratio of CD96 antagonist to PVRIG antagonist to TIGIT antagonist may be at least about 1:0.25:1.90, at least about 1:0.30:1.90, at least about 1:0.35:1.90, at least about 1:0.40:1.90, at least about 1:0.45:1.90, at least about 1:0.50:1.90, at least about 1:0.55:1.90, at least about 1:0.60:1.90, at least about 1:0.65:1.90, at least about 1:0.70:1.90, at least about 1:0.75:1.90, at least about 1:0.80:1.90, at least about 1:0.85:1.90, at least about 1:0.90:1.90, at least about 1:0.95:1.90, at least about 1:1:1.90, at least about 1:1.05:1.90, at least about 1:1.10:1.90, at least about 1:1.15:1.90, at least about 1:1.20:1.90, at least about 1:1.25:1.90, at least about 1:1.30:1.90, at least about 1:1.35:1.90, at least about 1:1.40:1.90, at least about 1:1.45:1.90, at least about 1:1.50:1.90, at least about 1:1.55:1.90, at least about 1:1.60:1.90, at least about 1:1.65:1.90, at least about 1:1.70:1.90, at least about 1:1.75:1.90, at least about 1:1.80:1.90, at least about 1:1.85:1.90, at least about 1:1.90:1.90, at least about 1:1.95:1.90, or at least about 1:2:1.90.

In a further embodiment, the ratio of CD96 antagonist to PVRIG antagonist to TIGIT antagonist may be at least about 1:0.25:1.95, at least about 1:0.30:1.95, at least about 1:0.35:1.95, at least about 1:0.40:1.95, at least about 1:0.45:1.95, at least about 1:0.50:1.95, at least about 1:0.55:1.95, at least about 1:0.60:1.95, at least about 1:0.65:1.95, at least about 1:0.70:1.95, at least about 1:0.75:1.95, at least about 1:0.80:1.95, at least about 1:0.85:1.95, at least about 1:0.90:1.95, at least about 1:0.95:1.95, at least about 1:1:1.95, at least about 1:1.05:1.95, at least about 1:1.10:1.95, at least about 1:1.15:1.95, at least about 1:1.20:1.95, at least about 1:1.25:1.95, at least about 1:1.30:1.95, at least about 1:1.35:1.95, at least about 1:1.40:1.95, at least about 1:1.45:1.95, at least about 1:1.50:1.95, at least about 1:1.55:1.95, at least about 1:1.60:1.95, at least about 1:1.65:1.95, at least about 1:1.70:1.95, at least about 1:1.75:1.95, at least about 1:1.80:1.95, at least about 1:1.85:1.95, at least about 1:1.90:1.95, at least about 1:1.95:1.95, or at least about 1:2:1.95.

In a further embodiment, the ratio of CD96 antagonist to PVRIG antagonist to TIGIT antagonist may be at least about 1:0.25:2, at least about 1:0.30:2, at least about 1:0.35:2, at least about 1:0.40:2, at least about 1:0.45:2, at least about 1:0.50:2, at least about 1:0.55:2, at least about 1:0.60:2, at least about 1:0.65:2, at least about 1:0.70:2, at least about 1:0.75:2, at least about 1:0.80:2, at least about 1:0.85:2, at least about 1:0.90:2, at least about 1:0.95:2, at least about 1:1:2, at least about 1:1.05:2, at least about 1:1.10:2, at least about 1:1.15:2, at least about 1:1.20:2, at least about 1:1.25:2, at least about 1:1.30:2, at least about 1:1.35:2, at least about 1:1.40:2, at least about 1:1.45:2, at least about 1:1.50:2, at least about 1:1.55:2, at least about 1:1.60:2, at least about 1:1.65:2, at least about 1:1.70:2, at least about 1:1.75:2, at least about 1:1.80:2, at least about 1:1.85:2, at least about 1:1.90:2, at least about 1:1.95:2, or at least about 1:2:2.

Also provided are compostions comprising a combinations disclosed herein. In one embodiment, a composition may comprise a PD-1 antagonist and a TIGIT antagonist. In another embodiment, a composition may comprise a PD-1 antagonist and a PVRIG antagonist. In another embodiment, a composition may comprise a PD-1 antagonist and a CD96 antagonist. In another embodiment, a composition may comprise a TIGIT antagonist and a PVRIG antagonist. In another embodiment, a composition may comprise a TIGIT antagonist and a CD96 antagonist. In another embodiment, a composition may comprise a PVRIG antagonist and a CD96 antagonist. In another embodiment, a composition may comprise a PD1 antagonist, a TIGIT antagonist, and a PVRIG antagonist. In another embodiment, a composition may comprise a PD1 antagonist, a TIGIT antagonist, and a CD96 antagonist. In another embodiment, a composition may comprise a PD1 antagonist, a TIGIT antagonist, and a PVRIG antagonist. In another embodiment, a composition may comprise a PD1 antagonist, a PVRIG antagonist, and a CD96 antagonist. In another embodiment, a composition may comprise a TIGIT antagonist, a PVRIG antagonist, and a CD96 antagonist.

Also provided are formulations comprising the combinations disclosed herein. In one embodiment, a formulation may comprise a PD-1 antagonist and a TIGIT antagonist. In another embodiment, a formulation may comprise a PD-1 antagonist and a PVRIG antagonist. In another embodiment, a formulation may comprise a PD-1 antagonist and a CD96 antagonist. In another embodiment, a formulation may comprise a TIGIT antagonist and a PVRIG antagonist. In another embodiment, a formulation may comprise a TIGIT antagonist and a CD96 antagonist. In another embodiment, a formulation may comprise a PVRIG antagonist and a CD96 antagonist. Such formulations may be lyophilized and resuspended in a pharmaceutically acceptable carrier prior to administration to a subject.

In another embodiment, a formulation may comprise a PD1 antagonist, a TIGIT antagonist, and a PVRIG antagonist. In another embodiment, a formulation may comprise a PD1 antagonist, a TIGIT antagonist, and a CD96 antagonist. In another embodiment, a formulation may comprise a PD1 antagonist, a TIGIT antagonist, and a PVRIG antagonist. In another embodiment, a formulation may comprise a PD1 antagonist, a PVRIG antagonist, and a CD96 antagonist. In another embodiment, a formulation may comprise a TIGIT antagonist, a PVRIG antagonist, and a CD96 antagonist. In a further embodiment, the formulation may be a liquid formulation or a lyophilized formulation.

In an embodiment, the formulation comprises a PD-1 antagonist, a TIGIT antagonist, a PVRIG, and/or a CD96 antagonist each at a concentration from at least about 1 mg/ml to at least about 100 mg/ml. In a further embodiment, the formulation may be a liquid formulation or a lyophilized formulation.

In one embodiment, the formulation comprises at least about 20 mg/ml of the PD1 antagonist, at least about 20 mg/ml of the TIGIT antagonist, 10 mM L-histidine buffer, at least about 7% w/v sucrose, at least about 0.02% w/v polysorbate 80, and at least about 10 mM L-methionine. In one embodiment, the formulation comprises at least about 25 mg/ml of the PD1 antagonist, at least about 25 mg/ml of the TIGIT antagonist, 10 mM L-histidine buffer, at least about 7% w/v sucrose, at least about 0.02% w/v polysorbate 80, and at least about 10 mM L-methionine. In one embodiment, the formulation comprises at least about 50 mg/ml of the PD1 antagonist, at least about 50 mg/ml of the TIGIT antagonist, 10 mM L-histidine buffer, at least about 7% w/v sucrose, at least about 0.02% w/v polysorbate 80, and at least about 10 mM L-methionine.

In one embodiment, the formulation comprises at least about 20 mg/ml of the PD1 antagonist, at least about 20 mg/ml of the TIGIT antagonist, at least about 20 mg/ml of the PVRIG antagonist, 10 mM L-histidine buffer, at least about 7% w/v sucrose, at least about 0.02% w/v polysorbate 80, and at least about 10 mM L-methionine. In one embodiment, the formulation comprises at least about 25 mg/ml of the PD1 antagonist, at least about 25 mg/ml of the TIGIT antagonist, at least about 25 mg/ml of the PVRIG antagonist, 10 mM L-histidine buffer, at least about 7% w/v sucrose, at least about 0.02% w/v polysorbate 80, and at least about 10 mM L-methionine. In one embodiment, the formulation comprises at least about 50 mg/ml of the PD1 antagonist, at least about 50 mg/ml of the TIGIT antagonist, at least about 50 mg/ml of the PVRIG antagonist, 10 mM L-histidine buffer, at least about 7% w/v sucrose, at least about 0.02% w/v polysorbate 80, and at least about 10 mM L-methionine.

In one embodiment, the formulation comprises at least about 20 mg/ml of the PD1 antagonist, at least about 20 mg/ml of the TIGIT antagonist, at least about 20 mg/ml of the CD96 antagonist, 10 mM L-histidine buffer, at least about 7% w/v sucrose, at least about 0.02% w/v polysorbate 80, and at least about 10 mM L-methionine. In one embodiment, the formulation comprises at least about 25 mg/ml of the PD1 antagonist, at least about 25 mg/ml of the TIGIT antagonist, at least about 25 mg/ml of the CD96 antagonist, 10 mM L-histidine buffer, at least about 7% w/v sucrose, at least about 0.02% w/v polysorbate 80, and at least about 10 mM L-methionine. In one embodiment, the formulation comprises at least about 50 mg/ml of the PD1 antagonist, at least about 50 mg/ml of the TIGIT antagonist, at least about 50 mg/ml of the CD96 antagonist, 10 mM L-histidine buffer, at least about 7% w/v sucrose, at least about 0.02% w/v polysorbate 80, and at least about 10 mM L-methionine.

In an embodiment, a formulation may comprise a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a formulation may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a formulation may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a formulation may comprise a PD-1 antagonist and a TIGIT antagonist wherein the PD-1 antagonist is dostarlimab and the TIGIT antagonist comprises an anti-TIGIT antibody comprising a heavy chain and a light chain variable sequence as shown in Table X.

In an embodiment, a formulation may comprise a PD-1 antagonist and a TIGIT antagonist wherein the PD-1 antagonist is dostarlimab and the TIGIT antagonist is vibostolimab.

In an embodiment, a formulation may comprise a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a formulation may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a formulation may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a formulation may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, a formulation may comprise a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a formulation may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a formulation may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a formulation may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, a formulation may comprise a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a formulation may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a formulation may comprise a TIGIT antagonist and a PVRIG antagonist wherein the TIGIT antagonist comprises an anti-TIGIT antibody comprising a heavy chain and a light chain variable sequence as shown in Table X and the PVRIG antagonist comprises VH comprising SEQ ID NO: 279 and VL comprising SEQ ID NO: 280.

In an embodiment, a formulation may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, a formulation may comprise a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a formulation may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a formulation may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a formulation may comprise a TIGIT antagonist and a CD96 antagonist wherein the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a formulation may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, a formulation may comprise a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a formulation may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a formulation may comprise a PVRIG antagonist and a CD96 antagonist wherein the PRVIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a formulation may comprise a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a formulation may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a formulation may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a formulation may comprise a PD-1 antagonist, a TIGIT antagonist, and a PVRIG antagonist wherein the PD-1 antagonist is dostarlimab, the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a formulation may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, a formulation may comprise a PD-1 antagonist, a TIGIT antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a formulation may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a formulation may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a formulation may comprise a PD-1 antagonist, a TIGIT antagonist, and a CD96 antagonist wherein the PD-1 antagonist is dostarlimab, the TIGIT antagonist comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a formulation may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, a formulation may comprise a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a formulation may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a formulation may comprise a PD-1 antagonist, a PVRIG antagonist, and a CD96 antagonist wherein the PD-1 antagonist comprises HC comprising SEQ ID NO: 160 and LC comprising SEQ ID NO: 161, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a formulation may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a formulation may comprise a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a formulation may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a formulation may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a formulation may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166 In an embodiment, a formulation may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

Treatment can be therapeutic, prophylactic or preventative. The subject will be one who is in need thereof. Those in need of treatment may include individuals already suffering from a particular medical disease, in addition to those who may develop the disease in the future.

Thus, the methods, antigen binding proteins and compositions of the disclosure described herein can be used for prophylactic treatment or preventative treatment if specified. In this case, methods, antigen binding proteins and compositions of the disclosure can be used to prevent or delay the onset of one or more aspects or symptoms of a disease. The subject can be asymptomatic. The subject may have a genetic predisposition to the disease. A prophylactically effective amount of the antigen binding protein is administered to such an individual. A prophylactically effective amount is an amount which prevents or delays the onset of one or more aspects or symptoms of a disease described herein.

The methods, antigen binding proteins and compositions of the disclosure need not affect a complete cure, or eradicate every symptom or manifestation of the disease to constitute a viable therapeutic treatment. As is recognised in the art, drugs employed as therapeutic agents in methods of treatment may reduce the severity of a given disease state, but need not abolish every manifestation of the disease to be regarded as useful therapeutic agents. Similarly, a prophylactically administered treatment need not be completely effective in preventing the onset of a disease in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur (for example by delaying the onset of the disease) or worsen in a subject, is sufficient.

Another aspect of the disclosure is a method of treatment of a disease in a subject in need thereof comprising administering to said subject a therapeutically effective amount of the CD96 binding protein or the pharmaceutical composition as described in any one of the preceding aspects to the subject. A further aspect of the disclosure is the method of treatment described in the preceding aspect further comprising whether the subject expresses CD96.

Another aspect of the disclosure is a CD96 binding protein or a pharmaceutical composition as described in any one of the preceding aspects for use in therapy or for use in the treatment of a disease.

Another aspect of the disclosure is a combination of two or more of a PD-1 antagonist, a TIGIT antagonist, a PVRIG antagonist, and/or CD96 antagonist or a pharmaceutical composition as described in any one of the preceding aspects for use in therapy or for use in the treatment of a disease. In one embodiment, a combination may comprise a PD-1 antagonist and a TIGIT antagonist. In another embodiment, a combination may comprise a PD-1 antagonist and a PVRIG antagonist. In another embodiment, a combination may comprise a PD-1 antagonist and a CD96 antagonist. In another embodiment, a combination may comprise a TIGIT antagonist and a PVRIG antagonist. In another embodiment, a combination may comprise a TIGIT antagonist and a CD96 antagonist. In another embodiment, a combination may comprise a PVRIG antagonist and a CD96 antagonist. In another embodiment, a combination may comprise a PD1 antagonist, a TIGIT antagonist, and a PVRIG antagonist. In another embodiment, a combination may comprise a PD1 antagonist, a TIGIT antagonist, and a CD96 antagonist. In another embodiment, a combination may comprise a PD1 antagonist, a TIGIT antagonist, and a PVRIG antagonist. In another embodiment, a combination may comprise a PD1 antagonist, a PVRIG antagonist, and a CD96 antagonist. In another embodiment, a combination may comprise a TIGIT antagonist, a PVRIG antagonist, and a CD96 antagonist.

In an embodiment, a combination for use in therapy or for use in the treatment of a disease for use in therapy or for use in the treatment of a disease may comprise a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a combination for use in therapy or for use in the treatment of a disease may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a combination for use in therapy or for use in the treatment of a disease may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a combination for use in therapy or for use in the treatment of a disease may comprise a PD-1 antagonist and a TIGIT antagonist wherein the PD-1 antagonist is dostarlimab and the TIGIT antagonist is comprises an anti-TIGIT antibody comprising a heavy chain and a light chain variable sequence as shown in Table X. In an embodiment, a combination for use in therapy or for use in the treatment of a disease may comprise a PD-1 antagonist and a TIGIT antagonist wherein the PD-1 antagonist is dostarlimab and the TIGIT antagonist is vibostolimab.

In an embodiment, a combination for use in therapy or for use in the treatment of a disease may comprise a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination for use in therapy or for use in the treatment of a disease may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a combination for use in therapy or for use in the treatment of a disease may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a combination for use in therapy or for use in the treatment of a disease may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, a combination for use in therapy or for use in the treatment of a disease may comprise a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in therapy or for use in the treatment of a disease may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a combination for use in therapy or for use in the treatment of a disease may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a combination for use in therapy or for use in the treatment of a disease may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, a combination for use in therapy or for use in the treatment of a disease may comprise a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination for use in therapy or for use in the treatment of a disease may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination for use in therapy or for use in the treatment of a disease may comprise a TIGIT antagonist and a PVRIG antagonist wherein the TIGIT antagonist is comprises an anti-TIGIT antibody comprising a heavy chain and a light chain variable sequence as shown in Table X and the PVRIG antagonist comprises VH comprising SEQ ID NO: 279 and VL comprising SEQ ID NO: 280. In an embodiment, a combination for use in therapy or for use in the treatment of a disease may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, a combination for use in therapy or for use in the treatment of a disease may comprise a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in therapy or for use in the treatment of a disease may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a combination for use in therapy or for use in the treatment of a disease may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a combination for use in therapy or for use in the treatment of a disease may comprise a TIGIT antagonist and a CD96 antagonist wherein the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a combination for use in therapy or for use in the treatment of a disease may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, a combination for use in therapy or for use in the treatment of a disease may comprise a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in therapy or for use in the treatment of a disease may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination for use in therapy or for use in the treatment of a disease may comprise a PVRIG antagonist and a CD96 antagonist wherein the PRVIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination for use in therapy or for use in the treatment of a disease may comprise a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination for use in therapy or for use in the treatment of a disease may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination for use in therapy or for use in the treatment of a disease may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination for use in therapy or for use in the treatment of a disease may comprise a PD-1 antagonist, a TIGIT antagonist, and a PVRIG antagonist wherein the PD-1 antagonist is dostarlimab, the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a combination for use in therapy or for use in the treatment of a disease may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, a combination for use in therapy or for use in the treatment of a disease may comprise a PD-1 antagonist, a TIGIT antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in therapy or for use in the treatment of a disease may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a combination for use in therapy or for use in the treatment of a disease may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a combination for use in therapy or for use in the treatment of a disease may comprise a PD-1 antagonist, a TIGIT antagonist, and a CD96 antagonist wherein the PD-1 antagonist is dostarlimab, the TIGIT antagonist comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a combination for use in therapy or for use in the treatment of a disease may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, a combination for use in therapy or for use in the treatment of a disease may comprise a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in therapy or for use in the treatment of a disease may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination for use in therapy or for use in the treatment of a disease may comprise a PD-1 antagonist, a PVRIG antagonist, and a CD96 antagonist wherein the PD-1 antagonist comprises HC comprising SEQ ID NO: 160 and LC comprising SEQ ID NO: 161, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination for use in therapy or for use in the treatment of a disease may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination for use in therapy or for use in the treatment of a disease may comprise a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in therapy or for use in the treatment of a disease may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination for use in therapy or for use in the treatment of a disease may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination for use in therapy or for use in the treatment of a disease may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination for use in therapy or for use in the treatment of a disease may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, the combination for use in therapy or for use in the treatment of a disease may comprise a PD-1 antagonist and a TIGIT antagonist wherein the PD-1 antagonist is pembrolizumab and the TIGIT antagonist is vibostolimab and the ratio of PD-1 antagonist to TIGIT antagonist may be at least about 1:0.80 to at least about 1:1.2. In a further embodiment, the PD-1 antagonist is pembrolizumab and the TIGIT antagonist is vibostolimab and the ratio of PD-1 antagonist to TIGIT antagonist may be at least about 1:0.80, at least about 1:0.85, at least about 1:0.90, at least about 1:0.95, at least about 1:1, at least about 1:1.05, at least about 1:1.10, at least about 1:1.15, or at least about 1:1.20. In yet a further embodiment, the PD-1 antagonist is pembrolizumab and the TIGIT antagonist is vibostolimab and the ratio of PD-1 antagonist to TIGIT antagonist may be 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, or 1:1.20. In yet another embodiment, the combination for use in therapy or for use in the treatment of a disease may comprise pembrolizumab and vibostolimab in a ratio of at least about 1:1.

In an embodiment, the combination for use in therapy or for use in the treatment of a disease may comprise a PD-1 antagonist and a TIGIT antagonist wherein the PD-1 antagonist is dostarlimab and the TIGIT antagonist is tiragolumab and the ratio of PD-1 antagonist to TIGIT antagonist may be at least about 1:0.80 to at least about 1:1.2. In a further embodiment, the PD-1 antagonist is dostarlimab and the TIGIT antagonist is tiragolumab and the ratio of PD-1 antagonist to TIGIT antagonist may be at least about 1:0.80, at least about 1:0.85, at least about 1:0.90, at least about 1:0.95, at least about 1:1, at least about 1:1.05, at least about 1:1.10, at least about 1:1.15, or at least about 1:1.20. In yet a further embodiment, the PD-1 antagonist is dostarlimab and the TIGIT antagonist is tiragolumab and the ratio of PD-1 antagonist to TIGIT antagonist may be 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, or 1:1.20. In yet another embodiment, the combination for use in therapy or for use in the treatment of a disease may comprise dostarlimab and tiragolumab in a ratio of at least about 1:1.

In an embodiment, the combination for use in therapy or for use in the treatment of a disease may comprise a PD-1 antagonist and a TIGIT antagonist wherein the PD-1 antagonist is dostarlimab and the TIGIT antagonist is vibostolimab and the ratio of PD-1 antagonist to TIGIT antagonist may be at least about 1:0.80 to at least about 1:1.2. In a further embodiment, the PD-1 antagonist is dostarlimab and the TIGIT antagonist is vibostolimab and the ratio of PD-1 antagonist to TIGIT antagonist may be at least about 1:0.80, at least about 1:0.85, at least about 1:0.90, at least about 1:0.95, at least about 1:1, at least about 1:1.05, at least about 1:1.10, at least about 1:1.15, or at least about 1:1.20. In yet a further embodiment, the PD-1 antagonist is dostarlimab and the TIGIT antagonist is vibostolimab and the ratio of PD-1 antagonist to TIGIT antagonist may be 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, or 1:1.20. In yet another embodiment, the combination for use in therapy or for use in the treatment of a disease may comprise dostarlimab and vibostolimab in a ratio of at least about 1:1.

In an embodiment, the combination for use in therapy or for use in the treatment of a disease may comprise a PD-1 antagonist and a TIGIT antagonist wherein the PD-1 antagonist is dostarlimab and the TIGIT antagonist is ASP8374 (PTZ-201) and the ratio of PD-1 antagonist to TIGIT antagonist may be at least about 1:0.80 to at least about 1:1.2. In a further embodiment, the PD-1 antagonist is dostarlimab and the TIGIT antagonist is ASP8374 (PTZ-201) and the ratio of PD-1 antagonist to TIGIT antagonist may be at least about 1:0.80, at least about 1:0.85, at least about 1:0.90, at least about 1:0.95, at least about 1:1, at least about 1:1.05, at least about 1:1.10, at least about 1:1.15, or at least about 1:1.20. In yet a further embodiment, the PD-1 antagonist is dostarlimab and the TIGIT antagonist is ASP8374 (PTZ-201) and the ratio of PD-1 antagonist to TIGIT antagonist may be 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, or 1:1.20. In yet another embodiment, the combination for use in therapy or for use in the treatment of a disease may comprise dostarlimab and ASP8374 (PTZ-201) in a ratio of at least about 1:1.

In an embodiment, the combination for use in therapy or for use in the treatment of a disease may comprise a PD-1 antagonist and a TIGIT antagonist wherein the PD-1 antagonist is dostarlimab and the TIGIT antagonist is EOS884448 and the ratio of PD-1 antagonist to TIGIT antagonist may be at least about 1:0.80 to at least about 1:1.2. In a further embodiment, the PD-1 antagonist is dostarlimab and the TIGIT antagonist is EOS884448 and the ratio of PD-1 antagonist to TIGIT antagonist may be at least about 1:0.80, at least about 1:0.85, at least about 1:0.90, at least about 1:0.95, at least about 1:1, at least about 1:1.05, at least about 1:1.10, at least about 1:1.15, or at least about 1:1.20. In yet a further embodiment, the PD-1 antagonist is dostarlimab and the TIGIT antagonist is EOS884448 and the ratio of PD-1 antagonist to TIGIT antagonist may be 1:0.80, 1:0.81, 1:0.82, 1:0.83, 1:0.84, 1:0.85, 1:0.86, 1:0.87, 1:0.88, 1:0.89, 1:0.90, 1:0.91, 1:0.92, 1:0.93, 1:0.94, 1:0.95, 1:0.96, 1:0.97, 1:0.98, 1:0.99, 1:1, 1:1.01, 1:1.02, 1:1.03, 1:1.04, 1:1.05, 1:1.06, 1:1.07, 1:1.08, 1:1.09, 1:1.10, 1:1.11, 1:1.12, 1:1.13, 1:1.14, 1:1.15, 1:1.16, 1:1.17, 1:1.18, 1:1.19, or 1:1.20. In yet another embodiment, the combination for use in therapy or for use in the treatment of a disease may comprise dostarlimab and EOS884448 in a ratio of at least about 1:1.

Another aspect of the disclosure is the use of a CD96 binding protein or a pharmaceutical composition as described in any one of the preceding claims in the manufacture of a medicament for use in the treatment of a disease.

Another aspect of the disclosure is the use of a combination of two or more of a PD-1 antagonist, a TIGIT antagonist, a PVRIG antagonist, and/or CD96 antagonist or a pharmaceutical composition as described above in the manufacture of a medicament for use in the treatment of a disease. In one embodiment, a combination may comprise a PD-1 antagonist and a TIGIT antagonist. In another embodiment, a combination may comprise a PD-1 antagonist and a PVRIG antagonist. In another embodiment, a combination may comprise a PD-1 antagonist and a CD96 antagonist. In another embodiment, a combination may comprise a TIGIT antagonist and a PVRIG antagonist. In another embodiment, a combination may comprise a TIGIT antagonist and a CD96 antagonist. In another embodiment, a combination may comprise a PVRIG antagonist and a CD96 antagonist. In another embodiment, a combination may comprise a PD1 antagonist, a TIGIT antagonist, and a PVRIG antagonist. In another embodiment, a combination may comprise a PD1 antagonist, a TIGIT antagonist, and a CD96 antagonist. In another embodiment, a combination may comprise a PD1 antagonist, a TIGIT antagonist, and a PVRIG antagonist. In another embodiment, a combination may comprise a PD1 antagonist, a PVRIG antagonist, and a CD96 antagonist. In another embodiment, a combination may comprise a TIGIT antagonist, a PVRIG antagonist, and a CD96 antagonist. In an embodiment, a combination for use in the manufacture of a medicament for use in the treatment of a disease for use in the manufacture of a medicament for use in the treatment of a disease may comprise a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a combination for use in the manufacture of a medicament for use in the treatment of a disease may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a combination for use in the manufacture of a medicament for use in the treatment of a disease may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a combination for use in the manufacture of a medicament for use in the treatment of a disease may comprise a PD-1 antagonist and a TIGIT antagonist wherein the PD-1 antagonist is dostarlimab and the TIGIT antagonist comprises an anti-TIGIT antibody comprising a heavy chain and a light chain variable sequence as shown in Table X. In an embodiment, a combination for use in the manufacture of a medicament for use in the treatment of a disease may comprise a PD-1 antagonist and a TIGIT antagonist wherein the PD-1 antagonist is dostarlimab and the TIGIT antagonist is vibostolimab.

In an embodiment, a combination for use in the manufacture of a medicament for use in the treatment of a disease may comprise a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination for use in the manufacture of a medicament for use in the treatment of a disease may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a combination for use in the manufacture of a medicament for use in the treatment of a disease may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a combination for use in the manufacture of a medicament for use in the treatment of a disease may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, a combination for use in the manufacture of a medicament for use in the treatment of a disease may comprise a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in the manufacture of a medicament for use in the treatment of a disease may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a combination for use in the manufacture of a medicament for use in the treatment of a disease may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a combination for use in the manufacture of a medicament for use in the treatment of a disease may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, a combination for use in the manufacture of a medicament for use in the treatment of a disease may comprise a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination for use in the manufacture of a medicament for use in the treatment of a disease may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination for use in the manufacture of a medicament for use in the treatment of a disease may comprise a TIGIT antagonist and a PVRIG antagonist wherein the TIGIT antagonist comprises an anti-TIGIT antibody comprising a heavy chain and a light chain variable sequence as shown in Table X and the PVRIG antagonist comprises VH comprising SEQ ID NO: 279 and VL comprising SEQ ID NO: 280. In an embodiment, a combination for use in the manufacture of a medicament for use in the treatment of a disease may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, a combination for use in the manufacture of a medicament for use in the treatment of a disease may comprise a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in the manufacture of a medicament for use in the treatment of a disease may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a combination for use in the manufacture of a medicament for use in the treatment of a disease may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a combination for use in the manufacture of a medicament for use in the treatment of a disease may comprise a TIGIT antagonist and a CD96 antagonist wherein the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a combination for use in the manufacture of a medicament for use in the treatment of a disease may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, a combination for use in the manufacture of a medicament for use in the treatment of a disease may comprise a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in the manufacture of a medicament for use in the treatment of a disease may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination for use in the manufacture of a medicament for use in the treatment of a disease may comprise a PVRIG antagonist and a CD96 antagonist wherein the PRVIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination for use in the manufacture of a medicament for use in the treatment of a disease may comprise a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination for use in the manufacture of a medicament for use in the treatment of a disease may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination for use in the manufacture of a medicament for use in the treatment of a disease may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination for use in the manufacture of a medicament for use in the treatment of a disease may comprise a PD-1 antagonist, a TIGIT antagonist, and a PVRIG antagonist wherein the PD-1 antagonist is dostarlimab, the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a combination for use in the manufacture of a medicament for use in the treatment of a disease may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, a combination for use in the manufacture of a medicament for use in the treatment of a disease may comprise a PD-1 antagonist, a TIGIT antagonist, and a CD96 antagonist wherein the PD-1 antagonist comprises CDRH1 comprising SEQ ID NO:

151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in the manufacture of a medicament for use in the treatment of a disease may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a combination for use in the manufacture of a medicament for use in the treatment of a disease may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a combination for use in the manufacture of a medicament for use in the treatment of a disease may comprise a PD-1 antagonist, a TIGIT antagonist, and a CD96 antagonist wherein the PD-1 antagonist is dostarlimab, the TIGIT antagonist comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a combination for use in the manufacture of a medicament for use in the treatment of a disease may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, a combination for use in the manufacture of a medicament for use in the treatment of a disease may comprise a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in the manufacture of a medicament for use in the treatment of a disease may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination for use in the manufacture of a medicament for use in the treatment of a disease may comprise a PD-1 antagonist, a PVRIG antagonist, and a CD96 antagonist wherein the PD-1 antagonist comprises HC comprising SEQ ID NO: 160 and LC comprising SEQ ID NO: 161, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination for use in the manufacture of a medicament for use in the treatment of a disease may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination for use in the manufacture of a medicament for use in the treatment of a disease may comprise a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination for use in the manufacture of a medicament for use in the treatment of a disease may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination for use in the manufacture of a medicament for use in the treatment of a disease may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination for use in the manufacture of a medicament for use in the treatment of a disease may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination for use in the manufacture of a medicament for use in the treatment of a disease may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

Another aspect of the disclosure is a pharmaceutical composition comprising a therapeutically effective amount of a CD96 binding protein as described in any one of the preceding aspects.

Another aspect of the disclosure is a pharmaceutical composition comprising a therapeutically effective amount of a combination of two or more of a PD-1 antagonist, a TIGIT antagonist, a PVRIG antagonist, and/or CD96 antagonist or the pharmaceutical composition as described in any one of the preceding aspects to the subject. In one embodiment, a combination may comprise a PD-1 antagonist and a TIGIT antagonist. In another embodiment, a combination may comprise a PD-1 antagonist and a PVRIG antagonist. In another embodiment, a combination may comprise a PD-1 antagonist and a CD96 antagonist. In another embodiment, a combination may comprise a TIGIT antagonist and a PVRIG antagonist. In another embodiment, a combination may comprise a TIGIT antagonist and a CD96 antagonist. In another embodiment, a combination may comprise a PVRIG antagonist and a CD96 antagonist. In another embodiment, a combination may comprise a PD1 antagonist, a TIGIT antagonist, and a PVRIG antagonist. In another embodiment, a combination may comprise a PD1 antagonist, a TIGIT antagonist, and a CD96 antagonist. In another embodiment, a combination may comprise a PD1 antagonist, a TIGIT antagonist, and a PVRIG antagonist. In another embodiment, a combination may comprise a PD1 antagonist, a PVRIG antagonist, and a CD96 antagonist. In another embodiment, a combination may comprise a TIGIT antagonist, a PVRIG antagonist, and a CD96 antagonist.

In an embodiment, a pharmaceutical composition comprising a pharmaceutical composition comprising a combination of antagonists of antagonists may comprise a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a pharmaceutical composition comprising a combination of antagonists may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a pharmaceutical composition comprising a combination of antagonists may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a pharmaceutical composition comprising a combination of antagonists may comprise a PD-1 antagonist and a TIGIT antagonist wherein the PD-1 antagonist is dostarlimab and the TIGIT antagonist comprises an anti-TIGIT antibody comprising a heavy chain and a light chain variable sequence as shown in Table X. In an embodiment, a pharmaceutical composition comprising a combination of antagonists may comprise a PD-1 antagonist and a TIGIT antagonist wherein the PD-1 antagonist is dostarlimab and the TIGIT antagonist is vibostolimab.

In an embodiment, a pharmaceutical composition comprising a combination of antagonists may comprise a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a pharmaceutical composition comprising a combination of antagonists may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, a pharmaceutical composition comprising a combination of antagonists may comprise a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a pharmaceutical composition comprising a combination of antagonists may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a pharmaceutical composition comprising a combination of antagonists may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, a pharmaceutical composition comprising a combination of antagonists may comprise a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a pharmaceutical composition comprising a combination of antagonists may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a pharmaceutical composition comprising a combination of antagonists may comprise a TIGIT antagonist and a PVRIG antagonist wherein the TIGIT antagonist comprises an anti-TIGIT antibody comprising a heavy chain and a light chain variable sequence as shown in Table X and the PVRIG antagonist comprises VH comprising SEQ ID NO: 279 and VL comprising SEQ ID NO: 280. In an embodiment, a pharmaceutical composition comprising a combination of antagonists may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, a pharmaceutical composition comprising a combination of antagonists may comprise a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a pharmaceutical composition comprising a combination of antagonists may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a pharmaceutical composition comprising a combination of antagonists may comprise a TIGIT antagonist and a CD96 antagonist wherein the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a pharmaceutical composition comprising a combination of antagonists may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, a pharmaceutical composition comprising a combination of antagonists may comprise a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a pharmaceutical composition comprising a combination of antagonists may comprise a PVRIG antagonist and a CD96 antagonist wherein the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a pharmaceutical composition comprising a combination of antagonists may comprise a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a pharmaceutical composition comprising a combination of antagonists may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist, a TIGIT antagonist, and a PVRIG antagonist wherein the PD-1 antagonist is dostarlimab, the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, a pharmaceutical composition comprising a combination of antagonists may comprise a PD-1 antagonist, a TIGIT antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a pharmaceutical composition comprising a combination of antagonists may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a pharmaceutical composition comprising a combination of antagonists may comprise a PD-1 antagonist, a TIGIT antagonist, and a CD96 antagonist wherein the PD-1 antagonist is dostarlimab, the TIGIT antagonist comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a pharmaceutical composition comprising a combination of antagonists may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, a pharmaceutical composition comprising a combination of antagonists may comprise a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a pharmaceutical composition comprising a combination of antagonists may comprise a PD-1 antagonist, a PVRIG antagonist, and a CD96 antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a pharmaceutical composition comprising a combination of antagonists may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a pharmaceutical composition comprising a combination of antagonists may comprise a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a pharmaceutical composition comprising a combination of antagonists may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a pharmaceutical composition comprising a combination of antagonists may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a pharmaceutical composition comprising a combination of antagonists may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. Another aspect of the disclosure is a method of treatment of a disease in a subject in need thereof comprising administering to said subject a therapeutically effective amount (e.g. a fixed amount as provided herein) of the combination of two or more of a PD-1 antagonist, a TIGIT antagonist, a PVRIG antagonist, and/or CD96 antagonist or the pharmaceutical composition as described in any one of the preceding aspects to the subject. In one embodiment, a combination may comprise a PD-1 antagonist and a TIGIT antagonist. In another embodiment, a combination may comprise a PD-1 antagonist and a PVRIG antagonist. In another embodiment, a combination may comprise a PD-1 antagonist and a CD96 antagonist. In another embodiment, a combination may comprise a TIGIT antagonist and a PVRIG antagonist. In another embodiment, a combination may comprise a TIGIT antagonist and a CD96 antagonist. In another embodiment, a combination may comprise a PVRIG antagonist and a CD96 antagonist. In another embodiment, a combination may comprise a PD1 antagonist, a TIGIT antagonist, and a PVRIG antagonist. In another embodiment, a combination may comprise a PD1 antagonist, a TIGIT antagonist, and a CD96 antagonist. In another embodiment, a combination may comprise a PD1 antagonist, a TIGIT antagonist, and a PVRIG antagonist. In another embodiment, a combination may comprise a PD1 antagonist, a PVRIG antagonist, and a CD96 antagonist. In another embodiment, a combination may comprise a TIGIT antagonist, a PVRIG antagonist, and a CD96 antagonist.

In an embodiment, a pharmaceutical composition comprising a combination of antagonists may comprise a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a pharmaceutical composition comprising a combination of antagonists may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a pharmaceutical composition comprising a combination of antagonists may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a pharmaceutical composition comprising a combination of antagonists may comprise a PD-1 antagonist and a TIGIT antagonist wherein the PD-1 antagonist is dostarlimab and the TIGIT antagonist comprises an anti-TIGIT antibody comprising a heavy chain and a light chain variable sequence as shown in Table X. In an embodiment, a pharmaceutical composition comprising a combination of antagonists may comprise a PD-1 antagonist and a TIGIT antagonist wherein the PD-1 antagonist is dostarlimab and the TIGIT antagonist is vibostolimab.

In an embodiment, a pharmaceutical composition comprising a combination of antagonists may comprise a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a pharmaceutical composition comprising a combination of antagonists may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, a pharmaceutical composition comprising a combination of antagonists may comprise a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a pharmaceutical composition comprising a combination of antagonists may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a pharmaceutical composition comprising a combination of antagonists may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, a pharmaceutical composition comprising a combination of antagonists may comprise a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a pharmaceutical composition comprising a combination of antagonists may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a pharmaceutical composition comprising a combination of antagonists may comprise a TIGIT antagonist and a PVRIG antagonist wherein the TIGIT antagonist comprises an anti-TIGIT antibody comprising a heavy chain and a light chain variable sequence as shown in Table X and the PVRIG antagonist comprises VH comprising SEQ ID NO: 279 and VL comprising SEQ ID NO: 280. In an embodiment, a pharmaceutical composition comprising a combination of antagonists may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, a pharmaceutical composition comprising a combination of antagonists may comprise a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a pharmaceutical composition comprising a combination of antagonists may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a pharmaceutical composition comprising a combination of antagonists may comprise a TIGIT antagonist and a CD96 antagonist wherein the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a pharmaceutical composition comprising a combination of antagonists may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, a pharmaceutical composition comprising a combination of antagonists may comprise a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a pharmaceutical composition comprising a combination of antagonists may comprise a PVRIG antagonist and a CD96 antagonist wherein the PRVIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a pharmaceutical composition comprising a combination of antagonists may comprise a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a pharmaceutical composition comprising a combination of antagonists may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist, a TIGIT antagonist, and a PVRIG antagonist wherein the PD-1 antagonist is dostarlimab, the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a pharmaceutical composition comprising a combination of antagonists may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, a pharmaceutical composition comprising a combination of antagonists may comprise a PD-1 antagonist, a TIGIT antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a pharmaceutical composition comprising a combination of antagonists may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a pharmaceutical composition comprising a combination of antagonists may comprise a PD-1 antagonist, a TIGIT antagonist, and a CD96 antagonist wherein the PD-1 antagonist is dostarlimab, the TIGIT antagonist comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a pharmaceutical composition comprising a combination of antagonists may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, a pharmaceutical composition comprising a combination of antagonists may comprise a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a pharmaceutical composition comprising a combination of antagonists may comprise a PD-1 antagonist, a PVRIG antagonist, and a CD96 antagonist wherein the PD-1 antagonist comprises HC comprising SEQ ID NO: 160 and LC comprising SEQ ID NO: 161, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a pharmaceutical composition comprising a combination of antagonists may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a pharmaceutical composition comprising a combination of antagonists may comprise a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a pharmaceutical composition comprising a combination of antagonists may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a pharmaceutical composition comprising a combination of antagonists may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a pharmaceutical composition comprising a combination of antagonists may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a pharmaceutical composition comprising a combination of antagonists may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

Another aspect of the disclosure is a method of treatment of a disease in a subject in need thereof comprising administering to said subject a therapeutically effective amount of the combination of two or more of a PD-1 antagonist, a TIGIT antagonist, a PVRIG antagonist, and/or CD96 antagonist or the pharmaceutical composition as described in any one of the preceding aspects to the subject. In one embodiment, a combination may comprise a PD-1 antagonist and a TIGIT antagonist. In another embodiment, a combination may comprise a PD-1 antagonist and a PVRIG antagonist. In another embodiment, a combination may comprise a PD-1 antagonist and a CD96 antagonist. In another embodiment, a combination may comprise a TIGIT antagonist and a PVRIG antagonist. In another embodiment, a combination may comprise a TIGIT antagonist and a CD96 antagonist. In another embodiment, a combination may comprise a PVRIG antagonist and a CD96 antagonist. In another embodiment, a combination may comprise a PD1 antagonist, a TIGIT antagonist, and a PVRIG antagonist. In another embodiment, a combination may comprise a PD1 antagonist, a TIGIT antagonist, and a CD96 antagonist. In another embodiment, a combination may comprise a PD1 antagonist, a TIGIT antagonist, and a PVRIG antagonist. In another embodiment, a combination may comprise a PD1 antagonist, a PVRIG antagonist, and a CD96 antagonist. In another embodiment, a combination may comprise a TIGIT antagonist, a PVRIG antagonist, and a CD96 antagonist.

Another aspect of the disclosure is a method of treatment of a disease in a subject in need thereof comprising administering to said subject a therapeutically effective amount of a PD-1 antagonist and a therapeutically effective amount of a combination of two or more of a TIGIT antagonist, a PVRIG antagonist, and/or CD96 antagonist to the subject. In an embodiment, a combination may comprise a TIGIT antagonist and a PVRIG antagonist. In another embodiment, a combination may comprise a TIGIT antagonist and a CD96 antagonist. In another embodiment, a combination may comprise a PVRIG antagonist and a CD96 antagonist. In another embodiment, a combination may comprise a TIGIT antagonist, a PVRIG antagonist, and a CD96 antagonist. In a further embodiment, the PD-1 antagonist is administered before, concurrently, or after the administration of the combination of two or more of a TIGIT antagonist, a PVRIG antagonist, and/or CD96 antagonist. In yet a further embodiment, the PD-1 antagonist is administered subcutaneously and the combination of two or more of a TIGIT antagonist, a PVRIG antagonist, and/or CD96 antagonist is administered intravenously. In another embodiment, the PD-1 antagonist is administered intravenously and the combination of two or more of a TIGIT antagonist, a PVRIG antagonist, and/or CD96 antagonist is administered intravenously. In another embodiment, the PD-1 antagonist is administered subcutaneously and the combination of two or more of a TIGIT antagonist, a PVRIG antagonist, and/or CD96 antagonist is administered subcutaneously.

In an embodiment, a combination used in a method of treatment of a disease used in a method of treatment of a disease may comprise a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a combination used in a method of treatment of a disease may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a combination used in a method of treatment of a disease may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a combination used in a method of treatment of a disease may comprise a PD-1 antagonist and a TIGIT antagonist wherein the PD-1 antagonist is dostarlimab and the TIGIT antagonist comprises an anti-TIGIT antibody comprising a heavy chain and a light chain variable sequence as shown in Table X. In an embodiment, a combination used in a method of treatment of a disease may comprise a PD-1 antagonist and a TIGIT antagonist wherein the PD-1 antagonist is dostarlimab and the TIGIT antagonist is vibostolimab.

In an embodiment, a combination used in a method of treatment of a disease may comprise a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination used in a method of treatment of a disease may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a combination used in a method of treatment of a disease may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a combination used in a method of treatment of a disease may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, a combination used in a method of treatment of a disease may comprise a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination used in a method of treatment of a disease may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a combination used in a method of treatment of a disease may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a combination used in a method of treatment of a disease may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, a combination used in a method of treatment of a disease may comprise a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination used in a method of treatment of a disease may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination used in a method of treatment of a disease may comprise a TIGIT antagonist and a PVRIG antagonist wherein the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a combination used in a method of treatment of a disease may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, a combination used in a method of treatment of a disease may comprise a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination used in a method of treatment of a disease may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a combination used in a method of treatment of a disease may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a combination used in a method of treatment of a disease may comprise a TIGIT antagonist and a CD96 antagonist wherein the TIGIT antagonist comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a combination used in a method of treatment of a disease may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, a combination used in a method of treatment of a disease may comprise a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination used in a method of treatment of a disease may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination used in a method of treatment of a disease may comprise a PVRIG antagonist and a CD96 antagonist wherein the PRVIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination used in a method of treatment of a disease may comprise a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination used in a method of treatment of a disease may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination used in a method of treatment of a disease may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination used in a method of treatment of a disease may comprise a PD-1 antagonist, a TIGIT antagonist, and a PVRIG antagonist wherein the PD-1 antagonist is dostarlimab, the TIGIT antagonist is comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a combination used in a method of treatment of a disease may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, a combination used in a method of treatment of a disease may comprise a PD-1 antagonist, a TIGIT antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination used in a method of treatment of a disease may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a combination used in a method of treatment of a disease may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a combination used in a method of treatment of a disease may comprise a PD-1 antagonist, a TIGIT antagonist, and a CD96 antagonist wherein the PD-1 antagonist is dostarlimab, the TIGIT antagonist is comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a combination used in a method of treatment of a disease may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, a combination used in a method of treatment of a disease may comprise a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination used in a method of treatment of a disease may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination used in a method of treatment of a disease may comprise a PD-1 antagonist, a PVRIG antagonist, and a CD96 antagonist wherein the PD-1 antagonist comprises HC comprising SEQ ID NO: 160 and LC comprising SEQ ID NO: 161, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination used in a method of treatment of a disease may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination used in a method of treatment of a disease may comprise a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination used in a method of treatment of a disease may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination used in a method of treatment of a disease may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination used in a method of treatment of a disease may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination used in a method of treatment of a disease may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

The present disclosure also provides a method of administering a combination of a PD-1 antagonist and a TIGIT antagonist to a patient in need thereof, comprising administering a combination of a first PD-1 antagonist and a first TIGIT antagonist to the patient in need thereof; ceasing administration of the combination of the first PD-1 antagonist and the first TIGIT antagonist to the patient in need thereof after a predetermined period of time; and subsequently administering a combination of a second PD-1 antagonist and a second TIGIT antagonist to the patient in need thereof, wherein the first PD-1 antagonist is different than the second PD-1 antagonist, and wherein the first TIGIT antagonist is different than the second TIGIT antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more. The present disclosure also provides a method of administering a combination of a PD-1 antagonist and a PVRIG antagonist to a patient in need thereof, comprising administering a combination of a first PD-1 antagonist and a first PVRIG antagonist to the patient in need thereof; ceasing administration of the combination of the first PD-1 antagonist and the first PVRIG antagonist to the patient in need thereof after a predetermined period of time; and subsequently administering a combination of a second PD-1 antagonist and a second PVRIG antagonist to the patient in need thereof, wherein the first PD-1 antagonist is different than the second PD-1 antagonist, and wherein the first PVRIG antagonist is different than the second PVRIG antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of administering a combination of a PD-1 antagonist and a CD96 antagonist to a patient in need thereof, comprising administering a combination of a first PD-1 antagonist and a first CD96 antagonist to the patient in need thereof; ceasing administration of the combination of the first PD-1 antagonist and the first CD96 antagonist to the patient in need thereof after a predetermined period of time; and subsequently administering a combination of a second PD-1 antagonist and a second CD96 antagonist to the patient in need thereof, wherein the first PD-1 antagonist is different than the second PD-1 antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of administering a combination of a TIGIT antagonist and a PVRIG antagonist to a patient in need thereof, comprising administering a combination of a first TIGIT antagonist and a first PVRIG antagonist to the patient in need thereof; ceasing administration of the combination of the first TIGIT antagonist and the first PVRIG antagonist to the patient in need thereof after a predetermined period of time; and subsequently administering a combination of a second TIGIT antagonist and a second PVRIG antagonist to the patient in need thereof, wherein the first TIGIT antagonist is different than the second TIGIT antagonist, and wherein the first PVRIG antagonist is different than the second PVRIG antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of administering a combination of a TIGIT antagonist and a CD96 antagonist to a patient in need thereof, comprising administering a combination of a first TIGIT antagonist and a first CD96 antagonist to the patient in need thereof; ceasing administration of the combination of the first TIGIT antagonist and the first CD96 antagonist to the patient in need thereof after a predetermined period of time; and subsequently administering a combination of a second TIGIT antagonist and a second CD96 antagonist to the patient in need thereof, wherein the first TIGIT antagonist is different than the second TIGIT antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of administering a combination of a PVRIG antagonist and a CD96 antagonist to a patient in need thereof, comprising administering a combination of a first PVRIG antagonist and a first CD96 antagonist to the patient in need thereof; ceasing administration of the combination of the first PVRIG antagonist and the first CD96 antagonist to the patient in need thereof after a predetermined period of time; and subsequently administering a combination of a second PVRIG antagonist and a second CD96 antagonist to the patient in need thereof, wherein the first PVRIG antagonist is different than the second PVRIG antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

In an embodiment, a combination may comprise a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a combination may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a combination may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a combination may comprise a PD-1 antagonist and a TIGIT antagonist wherein the PD-1 antagonist is dostarlimab and the TIGIT antagonist comprises an anti-TIGIT antibody comprising a heavy chain and a light chain variable sequence as shown in Table X. In an embodiment, a combination may comprise a PD-1 antagonist and a TIGIT antagonist wherein the PD-1 antagonist is dostarlimab and the TIGIT antagonist is vibostolimab.

In an embodiment, a combination may comprise a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a combination may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a combination may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, a combination may comprise a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a combination may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a combination may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, a combination may comprise a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a TIGIT antagonist and a PVRIG antagonist wherein the TIGIT antagonist comprises an anti-TIGIT antibody comprising a heavy chain and a light chain variable sequence as shown in Table X and the PVRIG antagonist comprises VH comprising SEQ ID NO: 279 and VL comprising SEQ ID NO: 280. In an embodiment, a combination may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, a combination may comprise a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a combination may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a combination may comprise a TIGIT antagonist and a CD96 antagonist wherein the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a combination may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, a combination may comprise a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination may comprise a PVRIG antagonist and a CD96 antagonist wherein the PRVIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination may comprise a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist is dostarlimab, the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, a combination may comprise a PD-1 antagonist, a TIGIT antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a combination may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a combination may comprise a PD-1 antagonist, a TIGIT antagonist, and a CD96 antagonist wherein the PD-1 antagonist is dostarlimab, the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a combination may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, a combination may comprise a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination may comprise a PD-1 antagonist, a PVRIG antagonist, and a CD96 antagonist wherein the PD-1 antagonist comprises HC comprising SEQ ID NO: 160 and LC comprising SEQ ID NO: 161, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, a combination may comprise a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ antagonist, and a CD96 antagonist wherein the TIGIT antagonist comprises VH comprising comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

The present disclosure also provides a method of administering a combination of a PD1 antagonist, a TIGIT antagonist, and a PVRIG antagonist to a patient in need thereof, comprising administering a combination of a first PD1 antagonist, a first TIGIT antagonist, and a first PVRIG antagonist to the patient in need thereof; ceasing administration of the combination of the first PD1 antagonist, the first TIGIT antagonist, and the first PVRIG antagonist to the patient in need thereof after a predetermined period of time; and subsequently administering a combination of a second PD1 antagonist, a second TIGIT antagonist, and a second PVRIG antagonist to the patient in need thereof, wherein the first PD1 antagonist is different than the second PD1 antagonist, wherein the first TIGIT antagonist is different than the second TIGIT antagonist, and wherein the first PVRIG antagonist is different than the second PVRIG antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of administering a combination of a PD1 antagonist, a TIGIT antagonist, and a CD96 antagonist to a patient in need thereof, comprising administering a combination of a first PD1 antagonist, a first TIGIT antagonist, and a first CD96 antagonist to the patient in need thereof; ceasing administration of the combination of the first PD1 antagonist, the first TIGIT antagonist, and the first CD96 antagonist to the patient in need thereof after a predetermined period of time; and subsequently administering a combination of a second PD1 antagonist, a second TIGIT antagonist, and a second CD96 antagonist to the patient in need thereof, wherein the first PD1 antagonist is different than the second PD1 antagonist, wherein the first TIGIT antagonist is different than the second TIGIT antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of administering a combination of a PD1 antagonist, a TIGIT antagonist, and a PVRIG antagonist to a patient in need thereof, comprising administering a combination of a first PD1 antagonist, a first TIGIT antagonist, and a first PVRIG antagonist to the patient in need thereof; ceasing administration of the combination of the first PD1 antagonist, the first TIGIT antagonist, and the first PVRIG antagonist to the patient in need thereof after a predetermined period of time; and subsequently administering a combination of a second PD1 antagonist, a second TIGIT antagonist, and a second PVRIG antagonist to the patient in need thereof, wherein the first PD1 antagonist is different than the second PD1 antagonist, wherein the first TIGIT antagonist is different than the second TIGIT antagonist, and wherein the first PVRIG antagonist is different than the second PVRIG antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of administering a combination of a PD1 antagonist, a PVRIG antagonist, and a CD96 antagonist to a patient in need thereof, comprising administering a combination of a first PD1 antagonist, a first PVRIG antagonist, and a first CD96 antagonist to the patient in need thereof; ceasing administration of the combination of the first PD1 antagonist, the first PVRIG antagonist, and the first CD96 antagonist to the patient in need thereof after a predetermined period of time; and subsequently administering a combination of a second PD1 antagonist, a second PVRIG antagonist, and a second CD96 antagonist to the patient in need thereof, wherein the first PD1 antagonist is different than the second PD1 antagonist, wherein the first PVRIG antagonist is different than the second PVRIG antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of administering a combination of a TIGIT antagonist, a PVRIG antagonist, and a CD96 antagonist to a patient in need thereof, comprising administering a combination of a first TIGIT antagonist, a first PVRIG antagonist, and a first CD96 antagonist to the patient in need thereof; ceasing administration of the combination of the first TIGIT antagonist, the first PVRIG antagonist, and the first CD96 antagonist to the patient in need thereof after a predetermined period of time; and subsequently administering a combination of a second TIGIT antagonist, a second PVRIG antagonist, and a second CD96 antagonist to the patient in need thereof, wherein the first TIGIT antagonist is different than the second TIGIT antagonist, wherein the first PVRIG antagonist is different than the second PVRIG antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

One embodiment of the disclosure is a method for the treatment of a disease in a subject (such as a human subject) in need thereof comprising administering to said subject a therapeutically effective amount of the CD96 binding protein as described in any one of the preceding embodiments, or the pharmaceutical composition described in any one of the preceding embodiments to the subject.

The present disclosure also provides a method of treating a disease or disorder (e.g., cancer) comprising: administering a therapeutically effective amount of a combination of a first PD-1 antagonist and a first TIGIT antagonist to the patient in need thereof; ceasing administration of the combination of the first PD-1 antagonist and the first TIGIT antagonist to the patient in need thereof after a predetermined period of time; and subsequently administering a therapeutically effective amount of a combination of a second PD-1 antagonist and a second TIGIT antagonist to the patient in need thereof, wherein the first PD-1 antagonist is different than the second PD-1 antagonist, and wherein the first TIGIT antagonist is different than the second TIGIT antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a combination of a first PD-1 antagonist and a first PVRIG antagonist to the patient in need thereof; ceasing administration of the combination of the first PD-1 antagonist and the first PVRIG antagonist to the patient in need thereof after a predetermined period of time; and subsequently administering a therapeutically effective amount of a combination of a second PD-1 antagonist and a second PVRIG antagonist to the patient in need thereof, wherein the first PD-1 antagonist is different than the second PD-1 antagonist, and wherein the first PVRIG antagonist is different than the second PVRIG antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a first PD-1 antagonist and a first CD96 antagonist to the patient in need thereof; ceasing administration of the combination of the first PD-1 antagonist and the first CD96 antagonist to the patient in need thereof after a predetermined period of time; and subsequently administering a therapeutically effective amount of a combination of a second PD-1 antagonist and a second CD96 antagonist to the patient in need thereof, wherein the first PD-1 antagonist is different than the second PD-1 antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a first TIGIT antagonist and a first PVRIG antagonist to the patient in need thereof; ceasing administration of the combination of the first TIGIT antagonist and the first PVRIG antagonist to the patient in need thereof after a predetermined period of time; and subsequently administering a therapeutically effective amount of a combination of a second TIGIT antagonist and a second PVRIG antagonist to the patient in need thereof, wherein the first TIGIT antagonist is different than the second TIGIT antagonist, and wherein the first PVRIG antagonist is different than the second PVRIG antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a first TIGIT antagonist and a first CD96 antagonist to the patient in need thereof; ceasing administration of the combination of the first TIGIT antagonist and the first CD96 antagonist to the patient in need thereof after a predetermined period of time; and subsequently administering a therapeutically effective amount of a combination of a second TIGIT antagonist and a second CD96 antagonist to the patient in need thereof, wherein the first TIGIT antagonist is different than the second TIGIT antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a first PVRIG antagonist and a first CD96 antagonist to the patient in need thereof; ceasing administration of the combination of the first PVRIG antagonist and the first CD96 antagonist to the patient in need thereof after a predetermined period of time; and subsequently administering a therapeutically effective amount of a combination of a second PVRIG antagonist and a second CD96 antagonist to the patient in need thereof, wherein the first PVRIG antagonist is different than the second PVRIG antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

In an embodiment, the method of treating a disease or disorder comprises administering a combination comprising a PD-1 antagonist and a TIGIT antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. In an embodiment, a combination may comprise a PD-1 antagonist and a TIGIT antagonist wherein the PD-1 antagonist comprises VH comprising SEQ ID NO: 158 and VL comprising SEQ ID NO: 159, and the TIGIT antagonist comprises VH comprising SEQ ID NO: 329 and VL comprising SEQ ID NO: 330. In an embodiment, a combination may comprise a PD-1 antagonist and a TIGIT antagonist wherein the PD-1 antagonist comprises HC comprising SEQ ID NO: 160 and LC comprising SEQ ID NO: 161, and the TIGIT antagonist comprises VH comprising SEQ ID NO: 329 and VL comprising SEQ ID NO: 330. In an embodiment, a combination may comprise a PD-1 antagonist and a TIGIT antagonist wherein the PD-1 antagonist is dostarlimab and the TIGIT antagonist comprises an anti-TIGIT antibody comprising a heavy chain and a light chain variable sequence as shown in Table X. In an embodiment, a combination may comprise a PD-1 antagonist and a TIGIT antagonist wherein the PD-1 antagonist is dostarlimab and the TIGIT antagonist is vibostolimab.

In an embodiment the method of treating a disease or disorder comprises administering a combination comprising a PD-1 antagonist and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a PD-1 antagonist and a PVRIG antagonist wherein the PD-1 antagonist comprises VH comprising SEQ ID NO: 158 and VL comprising SEQ ID NO: 159, and the PVRIG antagonist comprises VH comprising SEQ ID NO: 279 and VL comprising SEQ ID NO: 280. In an embodiment, a combination may comprise a PD-1 antagonist and a PVRIG antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280. In an embodiment, a combination may comprise a PD-1 antagonist and a PVRIG antagonist wherein the PD-1 antagonist is dostarlimab and the PVRIG antagonist comprises VH comprising SEQ ID NO: 279 and VL comprising SEQ ID NO: 280.

In an embodiment, the method of treating a disease or disorder comprises administering a combination comprising a PD-1 antagonist and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist and a CD96 antagonist wherein the PD-1 antagonist comprises VH comprising SEQ ID NO: 158 and VL comprising SEQ ID NO: 159, and the CD96 antagonist comprises VH comprising SEQ ID NO: 86 and VL comprising SEQ ID NO: 85. In an embodiment, a combination may comprise a PD-1 antagonist and a CD96 antagonist wherein the PD-1 antagonist comprises HC comprising SEQ ID NO: 160 and LC comprising SEQ ID NO: 161, and the CD96 antagonist comprises HC comprising SEQ ID NO: 165 and LC comprising SEQ ID NO: 166. In an embodiment, a combination may comprise a PD-1 antagonist and a CD96 antagonist wherein the PD-1 antagonist is dostarlimab and the CD96 antagonist comprises HC comprising SEQ ID NO: 165 and LC comprising SEQ ID NO: 166.

In an embodiment, the method of treating a disease or disorder comprises administering a combination comprising a TIGIT antagonist and a PVRIG antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a TIGIT antagonist and a PVRIG antagonist wherein the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a TIGIT antagonist and a PVRIG antagonist wherein the TIGIT antagonist comprises an anti-TIGIT antibody comprising a heavy chain and a light chain variable sequence as shown in Table X and the PVRIG antagonist comprises VH comprising SEQ ID NO: 279 and VL comprising SEQ ID NO: 280. In an embodiment, a combination may comprise a TIGIT antagonist and a PVRIG antagonist wherein the TIGIT antagonist is vibostolimab and the PVRIG antagonist comprises VH comprising SEQ ID NO: 279 and VL comprising SEQ ID NO: 280.

In an embodiment, the method of treating a disease or disorder comprises administering a combination comprising a TIGIT antagonist and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a TIGIT antagonist and a CD96 antagonist wherein the TIGIT antagonist comprises VH comprising SEQ ID NO: 329 and VL comprising SEQ ID NO: 330, and the CD96 antagonist comprises VH comprising SEQ ID NO: 86 and VL comprising SEQ ID NO: 85. In an embodiment, a combination may comprise a TIGIT antagonist and a CD96 antagonist wherein the TIGIT antagonist comprises VH comprising SEQ ID NO: 329 and VL comprising SEQ ID NO: 330, and the CD96 antagonist comprises HC comprising SEQ ID NO: 165 and LC comprising SEQ ID NO: 166. In an embodiment, a combination may comprise a TIGIT antagonist and a CD96 antagonist wherein the TIGIT antagonist comprises an anti-TIGIT antibody comprising a heavy chain and a light chain variable sequence as shown in Table X and the CD96 antagonist comprises HC comprising SEQ ID NO: 165 and LC comprising SEQ ID NO: 166. In an embodiment, a combination may comprise a TIGIT antagonist and a CD96 antagonist wherein the TIGIT antagonist is vibostolimab and the CD96 antagonist comprises HC comprising SEQ ID NO: 165 and LC comprising SEQ ID NO: 166.

In an embodiment, the method of treating a disease or disorder comprises administering a combination comprising a PVRIG antagonist and a CD96 antagonist wherein the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PVRIG antagonist and a CD96 antagonist wherein the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination may comprise a PVRIG antagonist and a CD96 antagonist wherein the PRVIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, the method of treating a disease or disorder comprises administering a combination comprising a PD-1 antagonist, a TIGIT antagonist, and a PVRIG antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. In an embodiment, a combination may comprise a PD-1 antagonist, a TIGIT antagonist, and a PVRIG antagonist wherein the PD-1 antagonist comprises VH comprising SEQ ID NO: 158 and VL comprising SEQ ID NO: 159, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist comprises HC comprising SEQ ID NO: 160 and LC comprising SEQ ID NO: 161, the TIGIT antagonist comprises VH comprising SEQ ID NO: 329 and VL comprising SEQ ID NO: 330, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist is dostarlimab, the TIGIT antagonist comprises an anti-TIGIT antibody comprising a heavy chain and a light chain variable sequence as shown in Table X, and the PVRIG antagonist comprises VH comprising SEQ ID NO: 279 and VL comprising SEQ ID NO: 280. In an embodiment, a combination may comprise a PD-1 antagonist, a TIGIT antagonist, and a PVRIG antagonist wherein the PD-1 antagonist is dostarlimab, the TIGIT antagonist is vibostolimab, and the PVRIG antagonist comprises VH comprising SEQ ID NO: 279 and VL comprising SEQ ID NO: 280.

In an embodiment, the method of treating a disease or disorder comprises administering a combination comprising a PD-1 antagonist, a TIGIT antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist, a TIGIT antagonist, and a CD96 antagonist wherein the PD-1 antagonist comprises VH comprising SEQ ID NO: 158 and VL comprising SEQ ID NO: 159, the TIGIT antagonist comprises VH comprising SEQ ID NO:329 and VL comprising SEQ ID NO: 330, and the CD96 antagonist comprises VH comprising SEQ ID NO: 86 and VL comprising SEQ ID NO: 85. In an embodiment, a combination may comprise a PD-1 antagonist, a TIGIT antagonist, and a CD96 antagonist wherein the PD-1 antagonist comprises HC comprising SEQ ID NO: 160 and LC comprising SEQ ID NO: 161, the TIGIT antagonist comprises VH comprising SEQ ID NO:329 and VL comprising SEQ ID NO: 330, and the CD96 antagonist comprises HC comprising SEQ ID NO: 165 and LC comprising SEQ ID NO: 166. In an embodiment, a combination may comprise a PD-1 antagonist, a TIGIT antagonist, and a CD96 antagonist wherein the PD-1 antagonist is dostarlimab, the TIGIT antagonist comprises an anti-TIGIT antibody comprising a heavy chain and a light chain variable sequence as shown in Table X, and the CD96 antagonist comprises HC comprising SEQ ID NO: 165 and LC comprising SEQ ID NO: 166. In an embodiment, a combination may comprise a PD-1 antagonist, a TIGIT antagonist, and a CD96 antagonist wherein the PD-1 antagonist is dostarlimab, the TIGIT antagonist is vibostolimab, and the CD96 antagonist comprises HC comprising SEQ ID NO: 165 and LC comprising SEQ ID NO: 166.

In an embodiment, the method of treating a disease or disorder comprises administering a combination comprising a PD-1 antagonist, a PVRIG antagonist, and a CD96 antagonist wherein the PD-1 antagonist comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a PD-1 antagonist, a PVRIG antagonist, and a CD96 antagonist wherein the PD-1 antagonist comprises VH comprising SEQ ID NO: 158 and VL comprising SEQ ID NO: 159, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination may comprise a PD-1 antagonist, a PVRIG antagonist, and a CD96 antagonist wherein the PD-1 antagonist comprises HC comprising SEQ ID NO: 160 and LC comprising SEQ ID NO: 161, and the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination may comprise a PD-1 antagonist, a PVRIG antagonist, and a CD96 antagonist wherein the PD-1 antagonist is dostarlimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

In an embodiment, the method of treating a disease or disorder comprises administering a combination comprising a TIGIT antagonist, a PVRIG antagonist, and a CD96 antagonist wherein the TIGIT antagonist comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333, the PVRIG antagonist comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228, and the CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. In an embodiment, a combination may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises VH comprising SEQ ID NO: 86 and VL comprising SEQ ID NO: 85. In an embodiment, a combination may comprise a TIGIT antagonist, a PVRIG antagonist, and a CD96 antagonist wherein the TIGIT antagonist comprises VH comprising SEQ ID NO: 329 and VL comprising SEQ ID NO: 330, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination may comprise a TIGIT antagonist, a PVRIG antagonist, and a CD96 antagonist wherein the TIGIT antagonist comprises an anti-TIGIT antibody comprising a heavy chain and a light chain variable sequence as shown in Table X, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166. In an embodiment, a combination may comprise a TIGIT antagonist, a PVRIG antagonist, and a CD96 antagonist wherein the TIGIT antagonist is vibostolimab, the PVRIG antagonist comprises VH comprising SEQ ID NO: 279 and VL comprising SEQ ID NO: 280, and the CD96 antagonist comprises HC comprising SEQ ID NO: 165 and LC comprising SEQ ID NO: 166.

The present disclosure also provides a method of treating a disease or disorder (e.g., cancer) comprising: administering a therapeutically effective amount of a combination of a first PD1 antagonist, a first TIGIT antagonist, and a first PVRIG antagonist to the patient in need thereof; ceasing administration of the combination of the first PD1 antagonist, the first TIGIT antagonist, and the first PVRIG antagonist to the patient in need thereof after a predetermined period of time; and subsequently administering a therapeutically effective amount of a combination of a second PD1 antagonist, a second TIGIT antagonist, and a second PVRIG antagonist to the patient in need thereof, wherein the first PD1 antagonist is different than the second PD1 antagonist, wherein the first TIGIT antagonist is different than the second TIGIT antagonist, and wherein the first PVRIG antagonist is different than the second PVRIG antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a first PD1 antagonist, a first TIGIT antagonist, and a first CD96 antagonist to the patient in need thereof; ceasing administration of the combination of the first PD1 antagonist, the first TIGIT antagonist, and the first CD96 antagonist to the patient in need thereof after a predetermined period of time; and subsequently administering a therapeutically effective amount of a combination of a second PD1 antagonist, a second TIGIT antagonist, and a second CD96 antagonist to the patient in need thereof, wherein the first PD1 antagonist is different than the second PD1 antagonist, wherein the first TIGIT antagonist is different than the second TIGIT antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a first PD1 antagonist, a first TIGIT antagonist, and a first PVRIG antagonist to the patient in need thereof; ceasing administration of the combination of the first PD1 antagonist, the first TIGIT antagonist, and the first PVRIG antagonist to the patient in need thereof after a predetermined period of time; and subsequently administering a therapeutically effective amount of a combination of a second PD1 antagonist, a second TIGIT antagonist, and a second PVRIG antagonist to the patient in need thereof, wherein the first PD1 antagonist is different than the second PD1 antagonist, wherein the first TIGIT antagonist is different than the second TIGIT antagonist, and wherein the first PVRIG antagonist is different than the second PVRIG antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a first PD1 antagonist, a first PVRIG antagonist, and a first CD96 antagonist to the patient in need thereof; ceasing administration of the combination of the first PD1 antagonist, the first PVRIG antagonist, and the first CD96 antagonist to the patient in need thereof after a predetermined period of time; and subsequently administering a therapeutically effective amount of a combination of a second PD1 antagonist, a second PVRIG antagonist, and a second CD96 antagonist to the patient in need thereof, wherein the first PD1 antagonist is different than the second PD1 antagonist, wherein the first PVRIG antagonist is different than the second PVRIG antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

The present disclosure also provides a method of treating a disease or disorder (e.g., cancer) comprising: administering a therapeutically effective amount of a combination of a first TIGIT antagonist, a first PVRIG antagonist, and a first CD96 antagonist to the patient in need thereof; ceasing administration of the combination of the first TIGIT antagonist, the first PVRIG antagonist, and the first CD96 antagonist to the patient in need thereof after a predetermined period of time; and subsequently administering a therapeutically effective amount of a combination of a second TIGIT antagonist, a second PVRIG antagonist, and a second CD96 antagonist to the patient in need thereof, wherein the first TIGIT antagonist is different than the second TIGIT antagonist, wherein the first PVRIG antagonist is different than the second PVRIG antagonist, and wherein the first CD96 antagonist is different than the second CD96 antagonist. In an embodiment, the predetermined period of time is at least about one week, at least about two weeks, at least about three weeks, at least about four weeks, at least about 5 weeks, at least about 7 weeks, at least about 8 weeks, at least about 9, weeks, at least about 10 weeks, at least about 11 weeks, at least about 12 weeks, at least about 4 months, at least about 5 months, at least about 6 months, at least about 7 months, at least about 8 months, at least about 9 months, at least about 10 months, at least about 11 months, or at least about 1 year or more.

One embodiment of the present disclosure is a method for the treatment of a disease in a subject in need thereof comprising administering to said subject a therapeutically effective amount of the CD96 binding or the pharmaceutical composition as described in any one of the preceding embodiments to the subject. A further embodiment of the present disclosure is a method for the treatment of a disease in a subject in need thereof further comprising determining whether the subject expresses CD96. An additional embodiment of the present disclosure is a CD96 binding protein or pharmaceutical as described in any one of the previous embodiments for use in therapy. An additional embodiment of the present disclosure is a CD96 binding protein or a pharmaceutical composition as described in any one of the preceding embodiments for use in the treatment of a disease. A further embodiment is the use of a CD96 binding protein or pharmaceutical composition described in any one of the preceding embodiments, in the manufacture of a medicament for use in the treatment of a disease. An additional embodiment of this disclosure is a method for the treatment of a disease, or a therapy, comprising administering to said subject a therapeutically effective amount of the CD96 binding or the pharmaceutical composition as described in any one of the preceding embodiments to the subject wherein the disease to be treated is a cancer. In a further aspect, the cancer is a solid tumour (e.g. a recurrent, metastatic or advanced solid tumour). The cancer can be: liver cancer (e.g. HCC), ovarian cancer, lung such as non-small cell lung cancer (NSCLC), renal cancer (e.g. RCC), colon cancer, gastric cancer, bladder cancer, head and neck squamous-cell carcinoma (HNSCC), or it can be leukemia, and/or any B cell malignancy.

### Embodiments

Embodiment 1 is a CD96 binding protein comprising:
(a) (i) any one or a combination of CDRs selected from CDRH1, CDRH2, CDRH3 from SEQ ID NOS: 2, 6, 10, 14, 18, 22, 26, 30, 34, 38, 42, 46, 50, 54, 58, 62, 66, 70, 74, 78, 82, 86, 90, and 94, and/or CDRL1, CDRL2, CRDL3 from SEQ ID NOS: 1, 5, 9, 13, 17, 21, 25, 29, 33, 37, 41, 45, 49, 53, 57, 61, 65, 69, 73, 77, 81, 85, 89, and 93; or (ii) a CDR variant of (i) wherein the variant has 1, 2, or 3 amino acid modifications; or
(b) a VH region comprising a sequence at least 80% identical to the sequence of SEQ ID NOS: 2, 6, 10, 14, 18, 22, 26, 30, 34, 38, 42, 46, 50, 54, 58, 62, 66, 70, 74, 78, 82, 86, 90, or 94; and/or a VL region comprising a sequence at least 80% identical to the sequence of SEQ ID NOS: 1, 5, 9, 13, 17, 21, 25, 29, 33, 37, 41, 45, 49, 53, 57, 61, 65, 69, 73, 77, 81, 85, 89, or 93.

Embodiment 2 is the CD96 binding protein according to embodiment 1, wherein the CDR of (a) (i) is:
(1) a CDRH1 selected from SEQ ID NOS: 105-125; a CDRH2 selected from SEQ ID NOS: 126-146; and/or a CDRH3 selected from SEQ ID NOS: 147-150; a CDRL1 selected from of SEQ ID NOS: 97-98; a CDRL2 selected from SEQ ID NOS: 99-100; and/or a CDRL3 selected from SEQ ID NOS: 101-104.

Embodiment 3 is the CD96 binding protein according to any of the preceding embodiments wherein the binding protein comprises CDRH3 that is 100% identical to Seq ID NOS: 147, 148, 149, or 150.

Embodiment 4 is the CD96 binding protein according to any of the preceding embodiments wherein the binding protein comprises: a CDRH1 that is 100% identical to SEQ ID NOS: 105, 106, 107, 108, 109, 110, 111, 112, 113, 114, 115, 116, 117, 118, 119, 120, 121, 122, 123, 124 or 125; a CDRH2 that is 100% identical to SEQ ID NOS: 126, 127, 128, 129, 130, 131, 132, 133, 134, 135, 136, 137, 138, 139, 140, 141, 142, 143, 144, 145, or 146; and/or a CDRH3 that is 100% identical to SEQ ID NOS: 147, 148, 149, or 150; a CDRL1 that is 100% identical to SEQ ID NO: 97 or 98; a CDRL2 that is 100% identical to SEQ ID NO: 99 or 100; and/or a CDRL3 that is 100% identical to SEQ ID NO: 101, 102, 103, or 104.

Embodiment 5 is the CD96 binding protein according to any one of the preceding embodiments wherein all 6 CDRs are present in the binding protein.

Embodiment 6 is a CD96 binding protein comprising the following 6 CDRs:
CDRH1 of SEQ ID NO: 115;
CDRH2 of SEQ ID NO: 145; and
CDRH3 of SEQ ID NO: 147; and
CDRL1 of SEQ ID NO: 97;
CDRL2 of SEQ ID NO: 99; and
CDRL3 of SEQ ID NO: 101.

Embodiment 7 is the CD96 binding protein according to embodiment 6 wherein the binding protein comprises:
a VH region that is 75% identical to SEQ ID NO: 86; and/or
a VL region that is 75% identical to SEQ ID NO: 85.

Embodiment 8 is the CD96 binding protein according to any one of the preceding embodiments wherein the binding protein comprises:
a VH region that is 100% identical to SEQ ID NO: 86; and/or
a VL region that is 100% identical to SEQ ID NO: 85.

Embodiment 9 is the CD96 binding protein according to any one of the preceding embodiments wherein the binding protein comprises a synthetic polypeptide, a humanised sequence, or a chimeric sequence.

Embodiment 10 is the CD96 binding protein according to any one of the preceding embodiments, wherein said protein binds to CD96 with a binding affinity (KD) which is equal to or better than that of CD155 or which can prevent or displace CD155 from binding to CD96.

Embodiment 11 is the CD96 binding protein according to any of the preceding embodiments, which binds to CD96 with a binding affinity (KD) of at least about 30 nM.

Embodiment 12 is the CD96 binding protein according to any one of the preceding embodiments, which is an antibody and which comprises an antibody Fc region which can bind to the Fc gamma receptor and/or can promote IFN gamma release.

Embodiment 13 is the CD96 binding protein according to embodiment 12, wherein said Fc region is the wild type IgG1Fc or a functional variant thereof.

Embodiment 14 is a CD96 binding protein which is an antibody and wherein the binding protein comprises: a heavy chain that is 100% identical to the amino acid sequence of SEQ ID NO: 165; and/or a light chain that is 100% identical to the amino acid sequence of SEQ ID NO: 166.

Embodiment 15 is a CD96 binding protein which is an antibody and wherein the binding protein comprises: a heavy chain that is 100% identical to the amino acid sequence of SEQ ID NO: 170; and/or a light chain that is 100% identical to the amino acid sequence of SEQ ID NO: 169.

Embodiment 16 is a CD96 binding protein which is an antibody and wherein the binding protein comprises: a heavy chain that is 100% identical to the amino acid sequence of SEQ ID NO: 174; and/or a light chain that is 100% identical to the amino acid sequence of SEQ ID NO: 173.

Embodiment 17 is a nucleic acid sequence which encodes the CD96 binding protein as defined in any one of the preceding embodiments.

Embodiment 18 is the nucleic acid sequence according to embodiment 17, wherein the sequence comprises SEQ ID NOS 4, 8, 12, 16, 20, 24, 28, 32, 36, 40, 44, 48, 52, 56, 60, 64, 68, 72, 76, 80, 84, 88, 92, 96, 167, 172, or 176 encoding the heavy chain; and/or SEQ ID NOS: 3, 7, 11, 15, 19, 23, 27, 31, 35, 39, 43, 47, 51, 55, 59, 63, 67, 71, 75, 79, 83, 87, 91, 95, 168, 171, or 175 encoding the light chain.

Embodiment 19 is an expression vector comprising the nucleic acid sequence as defined in embodiment 17 or 18.

Embodiment 20 is an expression vector according to embodiment 19, selected from the following: (i) a vector encoding the nucleic acid sequence of the heavy chain of SEQ ID NO 167 and the light chain of SEQ ID NO 168, (ii) a vector encoding the nucleic acid sequence of the heavy chain of SEQ ID NO 172 and the light chain of SEQ ID NO 171, and (iii) a vector encoding the nucleic acid sequence of the heavy chain of SEQ ID NO 176 and the light chain of SEQ ID NO 175.

Embodiment 21 is a recombinant host cell comprising the nucleic acid sequence(s) as defined in embodiment 17 or 18, or the expression vector(s) as defined in embodiments 19 or 20.

Embodiment 22 is a method for the production of an CD96 binding protein, which method comprises culturing the host cell as defined in embodiment 21 under conditions suitable for expression of said nucleic acid sequence(s) or vector(s), whereby a polypeptide comprising the CD96 binding protein is produced.

Embodiment 23 is the CD96 binding protein produced by the method of embodiment 22.

Embodiment 24 is a cell line engineered to express the CD96 binding protein of any one of embodiments 1 to 16.

Embodiment 25 is a pharmaceutical composition comprising the CD96 binding protein as defined in any one of embodiments 1 to 16 and a pharmaceutically acceptable excipient.

Embodiment 26 is a pharmaceutical composition according to embodiment 25 comprising the CD96 binding protein as defined in any of embodiments 6, 8 or 14-16 and a pharmaceutically acceptable excipient.

Embodiment 27 is a method for the treatment of a disease in a subject in need thereof comprising administering to said subject a therapeutically effective amount of the CD96 binding protein as defined in any one of embodiments 1 to 16, or the pharmaceutical composition as defined in any of embodiments 25-26

Embodiment 28 is a method for the treatment of a disease in a subject in need thereof comprising administering to said subject a therapeutically effective amount the CD96 binding protein as defined in any of embodiments 6, 8 or 14-16.

Embodiment 29 is the method of embodiment 27 or 28 further comprising determining whether the subject expresses CD96.

Embodiment 30 is a CD96 binding protein as defined in any one of embodiments 1 to 16, or a pharmaceutical composition as defined in any of embodiments 25-26 for use in therapy.

Embodiment 31 is a CD96 binding protein as defined in any one of embodiments 1 to 16 or a pharmaceutical composition as defined in any of embodiments 25-26 for use in the treatment of a disease in a subject.

Embodiment 32 is a CD96 binding protein for use according to embodiment 31 wherein said patient is a human subject

Embodiment 33 is a CD96 binding protein according to embodiment 31 or 32, for use in treating a solid tumour.

Embodiment 34 is a CD96 binding protein for use according to embodiment 31 or 32, wherein said protein is an antibody according to embodiment 14-16 and said solid tumour is selected from ovarian, lung (e.g. NSCLC), gastric, bladder, colorectal, liver (e.g. HCC), renal (e.g. RCC), and head and neck squamous cell carcinoma (HNSCC).

Embodiment 35 is use of a CD96 binding protein as defined in any one of embodiments 1 to 16, or a pharmaceutical composition as defined in any of embodiments 25-26, in the manufacture of a medicament for use in the treatment of a disease.

Embodiment 36 is a pharmaceutical composition according to claims any of embodiments 25-26, which further comprises a PD-1 antagonist and/or an anti-TIGIT antibody.

Embodiment 37 is a pharmaceutical composition according to embodiment 36, wherein said PD-1 antagonist is an antibody comprising a heavy chain sequence of SEQ ID NO:160 and a light chain sequence of SEQ ID NO:161.

Embodiment 38 is the method of embodiment 27 or 28 further comprising administering a PD-1 antagonist and/or an anti-TIGIT antibody.

Embodiment 39 is the method of embodiment 38 wherein said disease is cancer and wherein the CD96 binding protein and the PD-1 antagonist and/or anti-TIGIT antibody are administered to a patient such as a human patient in a route which is simultaneously or sequentially in any order.

Embodiment 40 is the method of embodiment 39 wherein said cancer is a solid tumour and wherein said PD1 antagonist is an antibody comprising a heavy chain sequence of SEQ ID NO:160 and a light chain sequence of SEQ ID NO:162.

Embodiment 41 is the method of embodiment 40 wherein said solid tumour is selected from ovarian, lung (e.g. NSCLC), gastric, bladder, colorectal, liver (e.g. HCC), renal (e.g. RCC), and head and neck squamous cell carcinoma (HNSCC).

Embodiment 42 is the method of embodiment 39, wherein the PD-1 antagonist and the anti-TIGIT antibody are a combination in a ratio of 1:0.25 to about 1:2 or a combination in a ratio of about 1.0:1.0.

Embodiment 43 is the method of embodiment 27 or 28 further comprising administering (a) a PD-1 antagonist, (b) a PVRIG antagonist (c) a TIGIT antagonist, or (d) a combination of two or more of (a), (b), or (c).

Embodiment 44 is the method of embodiment 43 wherein said disease is cancer and wherein the CD96 binding protein and the PD-1 antagonist and/or anti-PVRIG antagonist are administered to a patient such as a human patient in a route which is simultaneously or sequentially in any order.

Embodiment 45 is a method for treating cancer the method comprising: (1) administering an effective amount of a combination of a PD-1 antagonist, and a TIGIT antagonist in a ratio of about 1.0:0.80 to about 1.0:1.2 (PD-1 antagonist:TIGIT antagonist); (2) administering a effective amount of CD96 binding protein, the CD96 binding protein comprising a VH region comprising SEQ ID NO: 86; and a VL region comprising SEQ ID NO: 85.

Embodiment 46 is the method of embodiment 45 wherein the PD-1 antagonist of the combination comprises pembrolizumab or dostarlimab.

Embodiment 47 is the method of embodiment 45 wherein the TIGIT antagonist is selected from the group consisting of tiragolumab (MTIG7192A), vibostolimab (MK-7684), ASP8374 (PTZ-201), and EOS884448.

### EXAMPLES

### Example 1

### Generation of CD96 Binding Proteins

### Binding Protein Generation

Fully human antibodies specific for human CD96 were isolated from naïve eukaryotic libraries using biotinylated recombinant human and cynomolgus CD96 combined with cell sorting and FACS-based selection techniques. The heavy chain outputs from the naïve eukaryotic library selections were shuffled against a light chain library and further selections were performed to identify the optimal light chain pairings.

Affinity maturation was performed to improve antibody affinity/potency. This involved integrating the CDRH3 of antibody 42Y073-86F08-1 (GAGYYGDKDPMDV of SEQ ID NO: 2) into pre-made libraries with diversity in CDRH1 or CDRH2. Further selections were performed on this diversified library and the lead molecules were identified and sequenced. Amino acid and nucleic acid sequences of variable light and heavy chains for the lead molecules are shown in SEQ ID NOS: 1-96. Amino acid sequences of the CDR regions are shown in SEQ ID NOS: 97-150.

### Fc Selection

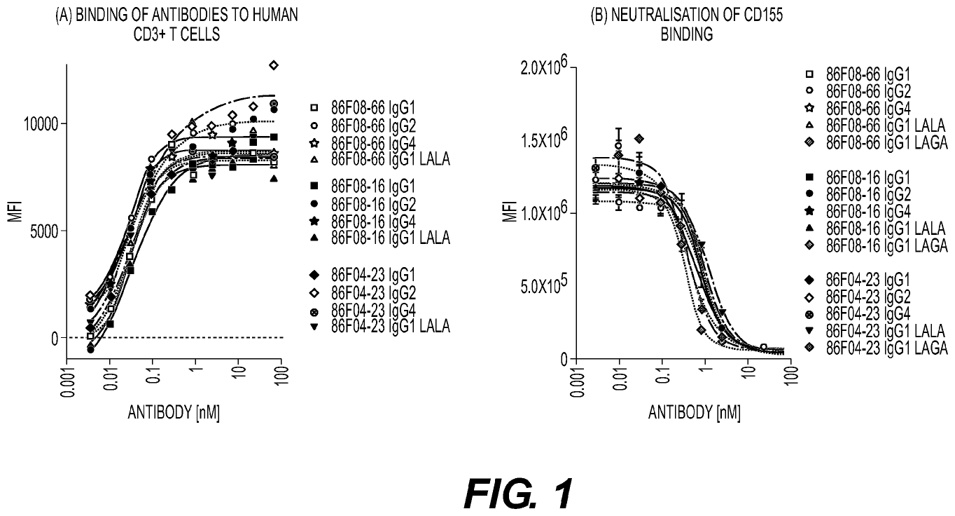

During the course of selection of lead molecules, the binding proteins were evaluated as human IgG1 WT molecules, or as Fc-disabled human IgG1 molecules. In order to make Fc-disabled molecules, amino acid residues at positions 234, 235, and 237, specifically, L234 and L235, or L235 and G237, were mutated to alanine, thus generating hIgG1 ‘LALA’ (L234A/L235A), and hIgG1 ‘LAGA’ (L235A/G237A) Fc-disabled human IgG1 molecules. These binding proteins exhibited CD96 binding and neutralisation of CD155 binding. Three CD96 binding proteins, 42Y073-86F08-66 (86F08-66), 42Y073-86F08-16 (86F08-16), and 42Y073-86F04-23 (86F04-23) were selected for further evaluation. The binding protein 42Y073-86F08-66 is also known as GSK 6097608 (a monoclonal antibody). These three CD96 binding proteins were evaluated with different IgG variants (human IgG1, IgG2, IgG4PE, or IgG1 Fc-disabled) for primary T cell binding, as well as CD155 neutralization activities. Variations in Fc did not affect binding to CD96-expressing cells, or neutralisation of CD155 binding to CD96-expressing cells, as indicated in FIG. 1.

### Example 2

### Characterization of CD96 Binding Proteins

### Binding to CHO Cells Expressing Human or Cynomolgus Monkey CD96

CHO cells expressing human CD96v2 or cynomolgus monkey CD96v2 were prepared in PBS. The cynomolgus monkey CD96v2 cells were stained with 1 μM violet proliferation dye (VPD450). Cells were diluted in pre-warmed media (RPMI containing 10% foetal calf serum) and incubated for a further 10 minutes at 37° C. before centrifugation at 400 g, 5 minutes and resuspension in flow buffer (PBS+0.5% BSA+2 mM EDTA) to 1.5×106/ml. The stained CHO-cyCD96 and unstained CHO-huCD96 cells were combined and added to wells of a 384-well assay plate together with dilutions of test anti-CD96 binding proteins for 1h at 4° C. Cells were further incubated with anti-IgG APC conjugate antibody and the amount of surface bound antibody on each of the CHO-cyCD96 and CHO-huCD96 cells was determined by flow cytometry. Median fluorescence intensity (MFI) values were fitted using a 4 parameter logistic model to calculate the EC50 of binding. Data is summarized in Table 2.

### Neutralisation of Human CD155 Binding to CHO Cells Expressing Human CD96v2

CD96 binding proteins were prepared in PBS in U-bottomed 96-well plates. CHO cells expressing human CD96v2 were added and incubated for 30 minutes at room temperature. After washing cells three times by centrifugation, human CD155-Fc-AF647 conjugate was added for a further 30 minutes at room temperature. Following three washes, cells were fixed and analysed for levels of CD155-Fc-AF647 staining by flow cytometry. Median fluorescence intensity (MFI) values were fitted using a 4 parameter logistic model to calculate IC50 values. Data is summarized in Table 2.

### Binding of CD96 Binding Proteins to Primary Human T Cells.

Human PBMCs were isolated from leukoreduction filters using density gradient centrifugation with Histopaque-1077. CD3⁺ T cells were subsequently isolated using a Pan T cell Isolation Kit (Miltenyi) following the manufacturer's instructions. Serial dilutions of test CD96 binding proteins were incubated with the human T cells for 1h at 4° C. Following three washes in buffer, anti-human IgG-APC conjugate was added for 1h at 4° C. Following three washes, cells were fixed and analysed for APC staining by flow cytometry. Median fluorescence intensity (MFI) values were fitted using a 4 parameter logistic model to calculate the EC50 of binding. Data is summarized in Table 2

TABLE 2
Binding data and neutralization of human CD155 binding to
hCD96v2 expressing CHO cells by CD96 binding proteins

| | | CHO-huCD96 | CHO-cyCD96 | Neutralization | T cell binding |
|---|---|---|---|---|---|
| Clone | Lineage | EC50 (nM) | EC50 (nM) | IC50 (nM) | EC50 (nM) |
| 42Y073-1A01-85 | 1A01 | 0.47 | 0.01 | 0.03 | 0.32 |
| 42Y073-1A01-97 | 1A01 | 0.77 | 0.02 | 0.03 | ND |
| 42Y073-1A01-100 | 1A01 | 0.02 | 0.04 | 0.06 | 0.01 |
| 42Y073-1A01-103 | 1A01 | 0.02 | 0.03 | 0.03 | 0.01 |
| 42Y073-1A01-126 | 1A01 | 0.02 | 0.03 | 0.10 | ND |
| 42Y073-1A01-191 | 1A01 | 0.01 | 0.02 | 0.03 | 0.38 |
| 42Y073-86F04-3 | 86F04 | 0.04 | 0.04 | 0.31 | 0.09 |
| 42Y073-86F04-4 | 86F04 | 0.04 | 0.07 | 0.18 | 0.05 |
| 42Y073-86F04-5 | 86F04 | 0.05 | 0.07 | 0.38 | 0.05 |
| 42Y073-86F04-6 | 86F04 | 0.06 | 0.11 | 0.25 | 0.01 |
| 42Y073-86F04-18 | 86F04 | 0.07 | 0.11 | 0.59 | 0.01 |
| 42Y073-86F04-23 | 86F04 | 0.06 | 0.03 | 0.76 | 0.01 |
| 42Y073-86F04-33 | 86F04 | 0.04 | 0.05 | 0.71 | 0.06 |
| 42Y073-86F04-88 | 86F04 | 0.05 | 0.16 | 0.21 | 0.06 |
| 42Y073-86F08-1 | 86F08 | 0.07 | 0.00 | 0.33 | 0.05 |
| 42Y073-86F08-3 | 86F08 | 0.04 | 0.08 | 0.26 | 0.05 |
| 42Y073-86F08-4 | 86F08 | 0.04 | 0.07 | 0.38 | 0.01 |
| 42Y073-86F08-8 | 86F08 | 0.17 | 0.03 | 0.93 | 0.04 |
| 42Y073-86F08-16 | 86F08 | 0.03 | 0.04 | 0.78 | 0.01 |
| 42Y073-86F08-17 | 86F08 | 0.10 | 0.03 | 0.52 | 0.01 |
| 42Y073-86F08-22 | 86F08 | 0.03 | 0.09 | 0.41 | 0.10 |
| 42Y073-86F08-47 | 86F08 | 0.05 | 0.06 | 0.17 | 0.00 |
| 42Y073-86F08-86 | 86F08 | 0.05 | 0.02 | 1.55 | 0.01 |
| 42Y073-2804-46 | 2B04 | 1.06 | 0.04 | 0.40 | ND |

### Selection of 42Y073-86F08-66 for Further Characterisation

To aid the selection of lead molecules, the biophysical properties of the anti-CD96 binding proteins were assessed. Namely the immunogenicity, deamidation, glycosylation, oxidation, aspartate isomerisation were predicted by in silico analysis and the aggregation, fragmentation and chemical degradation properties were assessed in the following conditions: 50 mM sodium phosphate pH 7.5 and 50 mM sodium acetate pH 5.0 at either 10 mg/ml or 1 mg/ml, and unstressed and stressed (thermal stress at 40° ° C. for 2 weeks) conditions. Anti-CD96 binding proteins were expressed in a HEK transient system and purified using Protein-A affinity and size exclusion chromatography. Anti-CD96 binding proteins were scored as follows:

TABLE 3
Sample preparation risk:
Percentage Recoveries

| Criteria | Low | Potential | High |
|---|---|---|---|
| Post-Dialysis | >80% | 80-70% | <70% |
| Post-Incubation | >98% | 95-98% | <95% |

TABLE 4
Aggregation & fragmentation risk by aSEC (analytical
size exclusion chromatography):

| Characterisitic | Low | Potential | High |
|---|---|---|---|
| Total % Monomer | >95.0% | 94-95% | <94% |
| Total % HMwS | <5.0% | 5.0-5.5% | >5.5% |
| Total % LMwS | <2.5% | 2.5-3% | >3% |

TABLE 5
Aggregation risk by DLS (dynamic light scattering):

| Characterisitic | Low | Potential | High |
|---|---|---|---|
| Average Rh* (nm) | 4.5-7 | 7-8 | >8 |
| Average % Mass | >98% | N/A | <98% |

*Rh = hydrodynamic radii

### Antigen Binding by BIACORE (Surface Plasmon Resonance):

A change in antigen binding within +10% between unstressed and stressed samples was considered an acceptable range (low risk). Molecules outside of this range could indicate a potential risk due to loss in binding activity.

Data is summarised in Tables 6 and 7. In Table 6, the position of the predicted risk in the sequence is identified (Kabat numbering). In Table 7, the following abbreviations are used for risks observed and the conditions under which they were observed: Agg=aggregation; incr. % Mass=increase in heterogenicity; Binding loss=a binding loss of greater than the assay limit was observed; Binding incr.=an increase in binding (indicates a possible propensity to aggregate); HMwS=High molecular weight species; LMwS=Low molecular weight species; Rh=hydrodynamic radius (where a larger radius indicates possible aggregation); PBS=phosphate buffer saline; U=unstressed; S=stressed; A=50 mM sodium acetate pH 5.0; and P=50 mM sodium phosphate pH 7.5.

42Y073-86F08-66, 42Y073-86F08-16 and 42Y073-86F04-23 demonstrated minimal development liabilities and minimal in silico risks. However, 42Y073-86F08-66 was selected for progression and further characterisation because the following attributes were observed:
An acceptable level of percentage total high molecular weight species (HMwS; <5%), low molecular weight species (LMwS; <2.5%) and monomer (95%), as determined by analytical size exclusion chromatography (aSEC);
Expected values for size distribution (>98% in Peak 2 of SEC) and hydrodynamic radius (4.5-7 nm), as determined by dynamic light scattering (DLS);
Minimal unexpected changes to chemical heterogeneity between unstressed and stressed as determined by capillary isoelectric focusing (cIEF); and
For antigen binding <10% change was determined by the antigen binding assay.

TABLE 6
In silico biophysical analysis results of CD96 binding proteins

| | Oxidation | De-amidation | Isomerization | Other |
|---|---|---|---|---|
| 42Y073-86F04-3 | Potential | Low | Low | Low |
| | (H_cdr2:54) | | | |
| 42Y073-86F04-5 | Low | Low | Low | Low |
| 42Y073-86F04-18 | Potential | Low | Low | Low |
| | (H_cdr2:54) | | | |
| 42Y073-86F04-23 | Potential | Low | Low | Low |
| | (H_cdr2:54) | | | |
| 42Y073-86F04-33 | Low | Low | Low | Low |
| 42Y073-86F08-3 | Low | Low | Low | Potential |
| | | | | (DP motif |
| | | | | H_cdr3:100C) |
| 42Y073-86F08-4 | Low | Low | Low | Potential |
| | | | | (DP motif |
| | | | | H_cdr3:100C) |
| 42Y073-86F08-16 | Low | Low | Low | Potential |
| | | | | (DP motif |
| | | | | H_cdr3:100C) |
| 42Y073-86F08-22 | Low | Low | Low | Potential |
| | | | | (DP motif |
| | | | | H_cdr3:100C) |
| 42Y073-86F08-47 | Low | Low | Low | Potential |
| | | | | (DP motif |
| | | | | H_cdr3:100C) |
| 42Y073-2B04-46 | Low | Low | Potential | Low |
| | | | (H_cdr2:52) | |
| 42Y073-1A01-85 | Low | Low | Low | Potential |
| | | | | (DP motif |
| | | | | H_cdr3:101) |
| 42Y073-1A01-100 | Low | Low | Low | Potential |
| | | | | (DP motif |
| | | | | H_cdr3:101) |
| 42Y073-1A01-103 | Low | Low | Low | Potential |
| | | | | (DP motif |
| | | | | H_cdr3:101) |
| 42Y073-1A01-126 | Low | Low | Low | Potential |
| | | | | (DP motif |
| | | | | H_cdr3:101) |
| 42Y073-86F08-66 | Low | Low | Low | Potential |
| | (H_cdr3:100E, | | | (DP motif |
| | H_fr2:48) | | | H_cdr3:100C) |
| 42Y073-86F08-16 | Low | Low | Low | Potential |
| | | | | (DP motif |
| | | | | H_cdr3:100C) |
| 42Y073-86F04-23 | Potential | Low | Low | Low |
| | (H_cdr2:54) | | | |

TABLE 7
Further biophysical analysis results of CD96 binding proteins

| | | Aggregation & | | | |
|---|---|---|---|---|---|
| | Sample | Fragmentation | Aggregation | Antigen | |
| | preparation | (aSEC) | (DLS) | binding | cIEF |
| 42Y073-86F04-3 | Low | Low | High | Low | Low |
| | | | (Agg in U, S, | | |
| | | | A & P and | | |
| | | | incr. % Mass | | |
| | | | in U, S & P) | | |
| 42Y073-86F04-5 | Low | Low | Potential | Low | Low |
| | | | (Agg and incr. | | |
| | | | % Mass in U, | | |
| | | | S & P) | | |
| 42Y073-86F04-18 | Low | Low | Low | Low | Low |
| 42Y073-86F04-23 | Low | Low | Low | Low | Low |
| 42Y073-86F04-33 | Low | Low | Low | Low | Low |
| 42Y073-86F08-3 | Low | Low | Low | Potential | Low |
| | | | | (11% Binding | |
| | | | | loss in S & A) | |
| 42Y073-86F08-4 | Low | High | Low | High | Low |
| | | (HMwS in S & | | (37% Binding | |
| | | A) | | loss in S & A) | |
| 42Y073-86F08-16 | Low | Low | Low | Low | Low |
| | | | | (9.57% Binding | |
| | | | | incr. in S & P) | |
| 42Y073-86F08-22 | Low | High | Low | High | Low |
| | | (HMwS in S & | | (41% Binding | |
| | | A) | | loss in S & A) | |
| 42Y073-86F08-47 | Low | Low | Low | Potential | Low |
| | | | | (34% Binding | |
| | | | | incr. in S & P) | |
| 42Y073-2B04-46 | High | High | High | High | Low |
| | (Yield loss | (HMwS and | (Agg and incr. | (36% Binding | |
| | in PBS) | LMwS in U, S | % Mass in S, | loss in S & A) | |
| | | & A) | A) | | |
| 42Y073-1A01-85 | Low | Low | Low | Low | Low |
| | | | | (9.99% Binding | |
| | | | | loss in S & P) | |
| 42Y073-1A01-100 | Low | Low | Low | Low | Low |
| 42Y073-1A01-103 | Low | Low | Low | Low | Low |
| 42Y073-1A01-126 | Low | Low | Low | Potential | Low |
| | | | | (13% Binding | |
| | | | | loss in S & P) | |
| 42Y073-86F08-66 | Low | Low | Low | Potential | Low |
| | | | | (12% Binding | |
| | | | | loss in S & A) | |
| 42Y073-86F08-16 | Low | Low | Low | Low | Low |
| | | | | (Binding loss in | |
| | | | | A) | |
| 42Y073-86F04-23 | Potential | Low | Low | Low | Low |
| | (74.3% | (LMwS in S) | (Rh in U, S & | | |
| | recovery in | | P) | | |
| | U & A) | | | | |

### Epitope Binning of CD96 Binding Proteins

Epitope binning competition assays were conducted to determine the epitopes of CD96 to which the anti-CD96 binding proteins presented herein bind. Briefly, HuCD96-His was incubated with a first CD96 binding protein at room temperature for 1 hour, before measuring binding of a second binding protein captured on a Protein A sensor by BLI. If binding of the second CD96 binding protein was observed, the two binding proteins were deemed to be non-competitive and assigned to different epitope bins. If no binding of the second CD96 binding protein could be seen, the two binding proteins were deemed to be competitive and assigned to the same epitope bin. A self-binning control was included for each CD96 binding protein, using the same binding protein as both the first and the second binding protein.

Competition between all CD96 binding proteins was observed, except for 42Y073-2B04-46, indicating that all except 42Y073-2B04-46 bind similar epitopes of CD96. This data suggests that, with the exception of 42Y073-2B04-46, all CD96 binding proteins presented herein bind to the same epitope of CD96 or to spatially close epitopes such that binding of further CD96 binding proteins is inhibited (i.e. they belong to the same epitope bin). However, the finding that 42Y073-2B04-46 does not compete with the other CD96 binding proteins presented herein suggests that this clone binds a distinct epitope of CD96 (and thus belongs to a distinct epitope bin). Thus, together with the data presented in Table 2, it will be appreciated that the binding and neutralization of human CD155 binding to hCD96v2 expressing CHO cells by the CD96 binding proteins presented herein is not limited to the binding to any particular epitope of CD96.

### Further Binding Affinity Studies of CD96 Binding Proteins to CD96 Isoforms

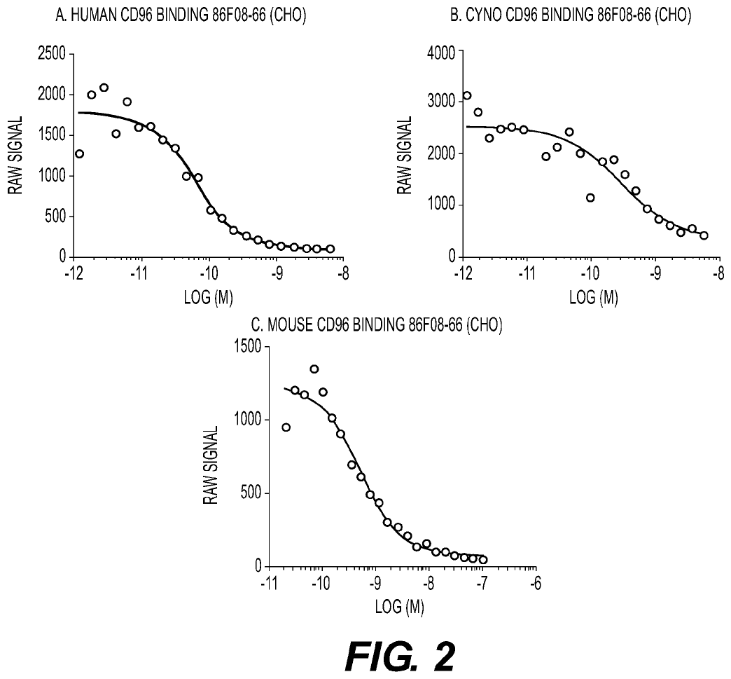

The binding affinity of 42Y073-86F08-66, a fully human IgG1 antibody with wild-type Fc, expressed and purified from CHO cells, to recombinant CD96 was determined using solution equilibrium titration (MSD-SET) assays. 42Y073-86F08-66 bound to recombinant human CD96v2 with a mean KD of 20 pM, and to recombinant cynomolgus monkey CD96v2 with a mean KD of 278 pM (FIG. 2). In addition, it was demonstrated that 42Y073-86F08-66 bound to recombinant murine CD96v2 with a mean KD of 479 pM.

In surface plasmon resonance (SPR) assays, 42Y073-86F08-66 did not bind to recombinant human CD155, human CD226, human TIGIT or human nectin-1, which are homologs in the pathway.

### MSD-Set Assays Procedure:

Summary: MSD-SET (MSD solution equilibrium titration) analysis was used in order to determine the affinities of these antibodies to human and mouse CD96 proteins at 25° ° C. as the dissociation rates were too slow to measure by BIACORE at this temperature. MSD-SET determines the solution phase, equilibrium affinity of antibodies. The method relies on the detection of free antigen at equilibrium in a titrated series of antibody concentrations.

Procedure:

Biotinylated human CD96 protein was used at a constant concentration of 1.5 nM and cynomolgus monkey biotinylated CD96 protein at 3 nM. Antibody samples were titrated 1 in 5 over a 22 point curve from 6 nM for the human CD96 and cynomolgus monkey CD96. The titrated antibody and CD96 protein were incubated for 24 h at room temperature. After 24 h, 5 nM antibodies were coated onto standard bind MSD plates (Meso Scale Discovery, L15XA) for 30 min at room temperature. Plates were then blocked with STARTING BLOCK blocking buffer (Thermo Scientific, #37542) for 30 min with shaking at 700 rpm, followed by three washes with wash buffer. The incubated solutions were added to the MSD plates for 150 s with shaking at 700 rpm followed by one wash. Antigen captured on a plate was detected with a SULFOTAG-labelled streptavidin (Meso Scale Discovery, R32AD-1) by incubation on the plate for 3 min. The plates were washed three times with wash buffer and then read on an MSD SECTOR IMAGER instrument using 1× Read Buffer T with surfactant (Meso Scale Discovery, R92TC-1). The percent free antigen was plotted as a function of titrated antibody using GRAPHPAD PRISM software and fitted to a quadratic equation.

### CD96 Binding Proteins Binding to HEK Cells Overexpressing CD96 Proteins

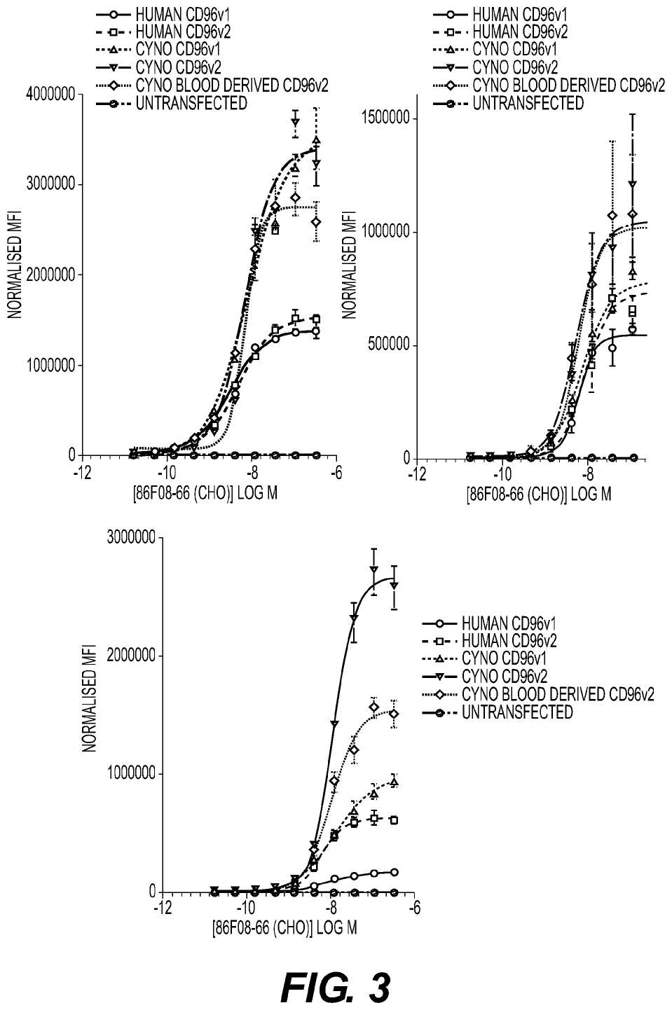

The binding of 42Y073-86F08-66 to the known isoforms of human or cynomolgus monkey CD96 on cell membranes was determined by flow cytometry using HEK cells that had been transfected with these different isoforms (FIG. 3). Despite the differences in maximum binding response to the expressed isoforms, and between experiments (most likely due to variation in expression efficiency), comparable binding activities as indicated by the EC50 of 42Y073-86F08-66 have been demonstrated to these CD96 isoforms (Table 8). Taken together these data suggest that 42Y073-86F08-66 is capable of recognising all membrane forms of CD96 presented in human and cynomolgus monkey.

TABLE 8
Potency of 42Y073-86F08-66 for binding to HEK cells
transiently transfected with CD96 isoforms

| | Human | Human | Cyno | Cyno | Blood derived |
|---|---|---|---|---|---|
| | CD96v1 | CD96v2 | CD96v1 | CD96v2 | Cyno CD96v2 |
| Geometric mean | 5.28 | 6.44 | 9.17 | (5.54-11.21) | 7.34 |
| EC50 (Range) | (3.13-8.89) | (4.72-8.78) | (6.83-12.3) | 7.88 | (5.27-10.23) |
| nM | | | | | |
| (n = 3) | | | | | |

### Binding Affinity of CD96 Binding Proteins to Native CD96 on Human and Cynomolgus Monkey T Cells

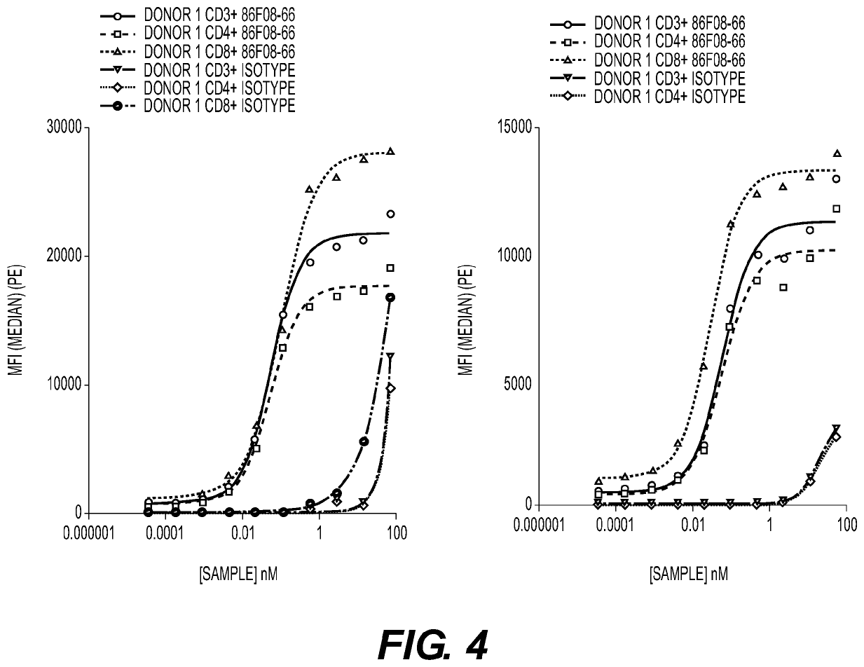

To confirm that 42Y073-86F08-66 binds to native CD96 expressed on primary human and monkey cells, the binding of 42Y073-86F08-66 to CD3⁺ T cells and subsets (CD4⁺ or CD8⁺) in both species was determined by flow cytometry. 42Y073-86F08-66 bound with high affinity to human CD4+ and CD8⁺ T cells (EC50s of 47 pm and 45 pM respectively) (FIG. 4 and Table 9). 42Y073-86F08-66 has a higher potency (pM) for binding to primary human T cells compared with HEK cells that over-express CD96 (nM) as a result of the relatively low expression of CD96 on primary cells compared with the expression of CD96 after transient transfection of CD96 isoforms under the control of a strong, constitutive promoter element.

TABLE 9
Potency of 42Y073-86F08-66 to primary human T cells (total
CD3⁺ T cells vs CD4⁺ or CD8⁺ subsets)

| | Human total | Human CD4⁺ | Human CD8⁺ |
|---|---|---|---|
| | CD3+ T cells | T cells | T cells |
| Geometric mean EC50 | 49.9 | 47.0 | 44.7 |
| (Range) pM | (39.6-62.6) | (37.7-58.5) | (31.6-63.3) |
| (n = 6) | | | |

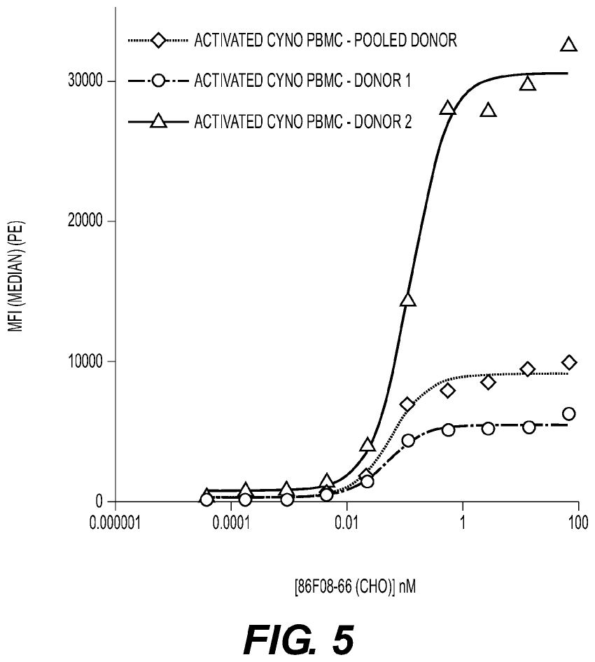

To confirm that 42Y073-86F08-66 binds to CD96 in cynomolgus monkeys, binding to primary monkey T cells was determined. Based on the binding of 42Y073-86F08-66 and a commercially available anti-CD96 antibody 6F9, cynomolgus monkey T cells were found to express much lower levels of CD96 compared with human T cells. A similar observation was made for rhesus monkey T cells. To robustly quantify CD96 target engagement, purified cynomolgus monkey CD3⁺ T cells were activated using anti-CD2/3/28 beads for 17 days to upregulate CD96 expression. 42Y073-86F08-66 binding was determined for 3 different activated T cell samples from cynomolgus monkeys, and 42Y073-86F08-66 binding was confirmed in all these samples. While there was a differential maximal signal among samples, the EC50s were very similar (mean 76.6 pM), and within 2-fold of that observed for human T cells (49.9 pM) (FIG. 5).

### Internalisation of CD96 Binding Proteins Upon Binding to Human T Cells and NK Cells

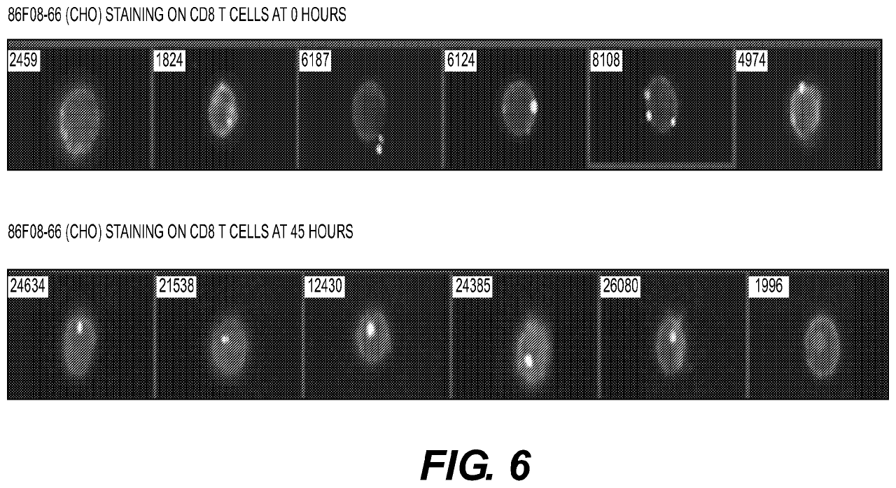

The fate of 42Y073-86F08-66 following binding was determined in human PBMC cultures from 3 donors using imaging cytometry. Highly punctate staining with 42Y073-86F08-66 was observed on the cell membrane in all 3 cell populations (CD4⁺ T cells, CD8⁺ T cells and NK cells) from all 3 donors at baseline, and this staining pattern remained across the 45 hour time-course (FIG. 6). In all three cell populations defined (CD4⁺, CD8⁺ and NK cells) the internalisation of 42Y073-86F08-66-PE was slow, and not complete over a 45 hour time course. These data suggest that 42Y073-86F08-66 internalisation is slow relative to other antibodies that target T cell surface receptors.

### CD96 Binding Proteins Inhibits Binding of CD96 Ligand CD155 to CD96-Expressing Cells

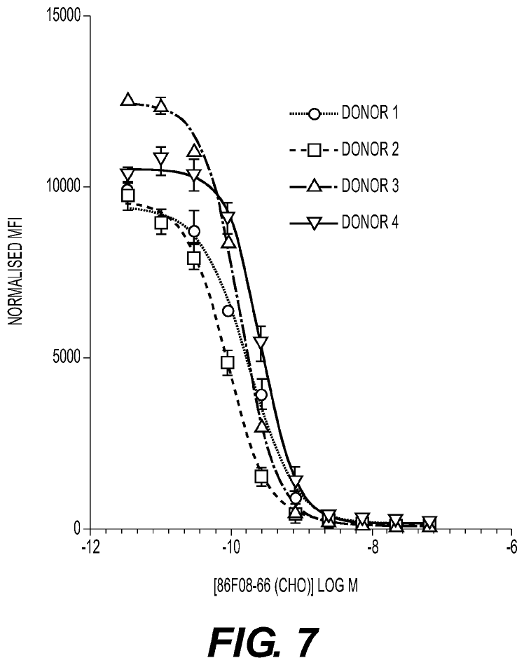

In primary human T cells it was demonstrated that pre-complexation of 42Y073-86F08-66 to membrane CD96 inhibited the latter from binding to its ligand (recombinant human CD155:Fc) (IC50 0.16 nM) (FIG. 7).

### Disruption of Established CD96:CD155 Interactions by CD96 Binding Proteins

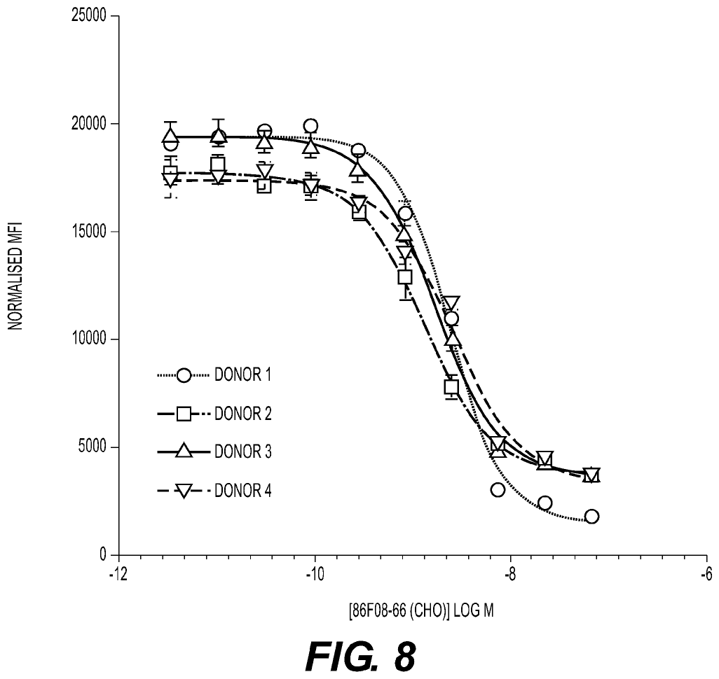

When CD155:Fc was pre-bound to primary human T cells, 42Y073-86F08-66 could compete-off CD155 in a dose-dependent manner (IC50 1.93 nM) (FIG. 8), indicating that 42Y073-86F08-66 can displace CD96-bound CD155, the natural ligand for CD96.

### Fc Receptor Engagement by CD96 Binding Proteins

Since 42Y073-86F08-66 is a fully human IgG1 antibody with a WT Fc, it is expected to bind to relevant Fc receptors.

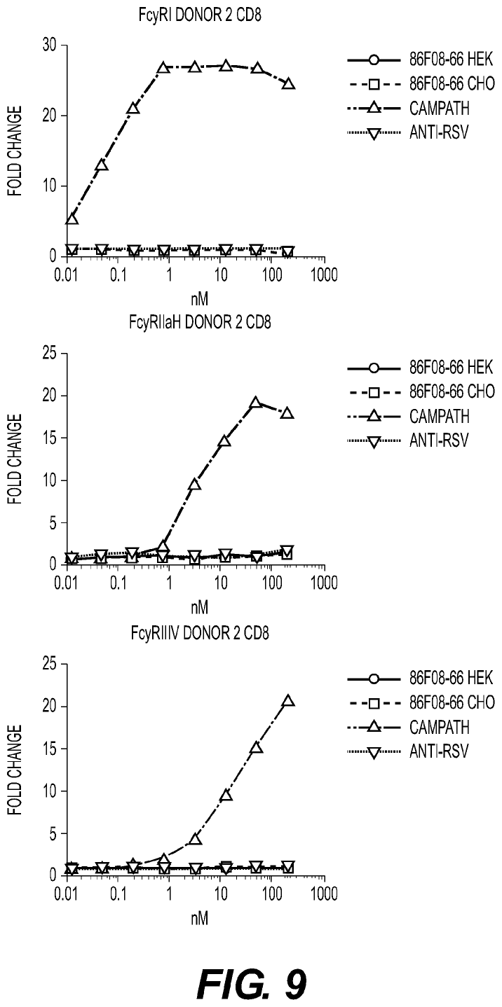

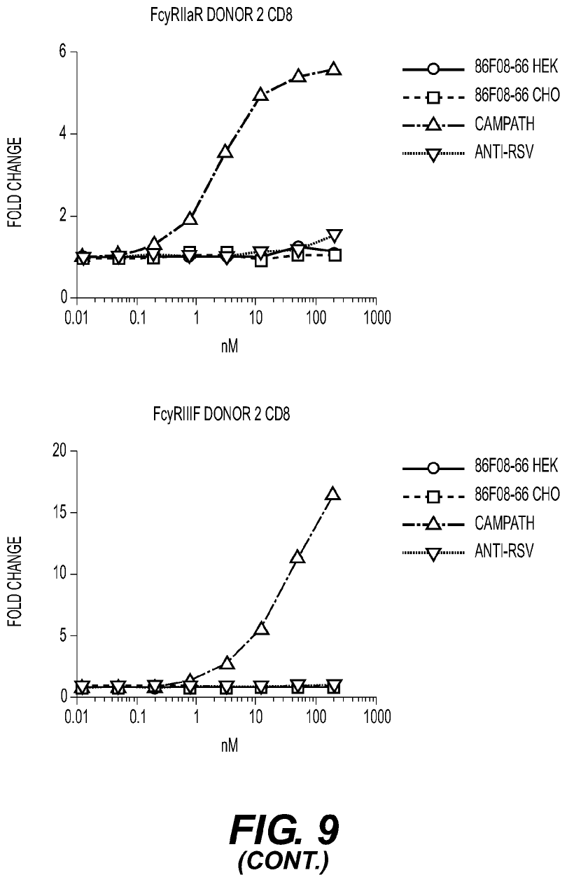

Using a panel of recombinant cell lines (Promega) expressing activatory human Fcγ receptor reporters (FcγRI, FcγRIIa(R), FcγRIIa(H), FcγRIIa(V) and FcγRIIIa(F)) we demonstrated that the binding of 42Y073-86F08-66 to CD96 on primary human T cells did not result in the activation of human Fcγ receptors (FIG. 9). In contract, a control anti-CD52 antibody (Campath) efficiently elicited FcγR activation signals with all of the Fcγ receptor-expressing cells tested.

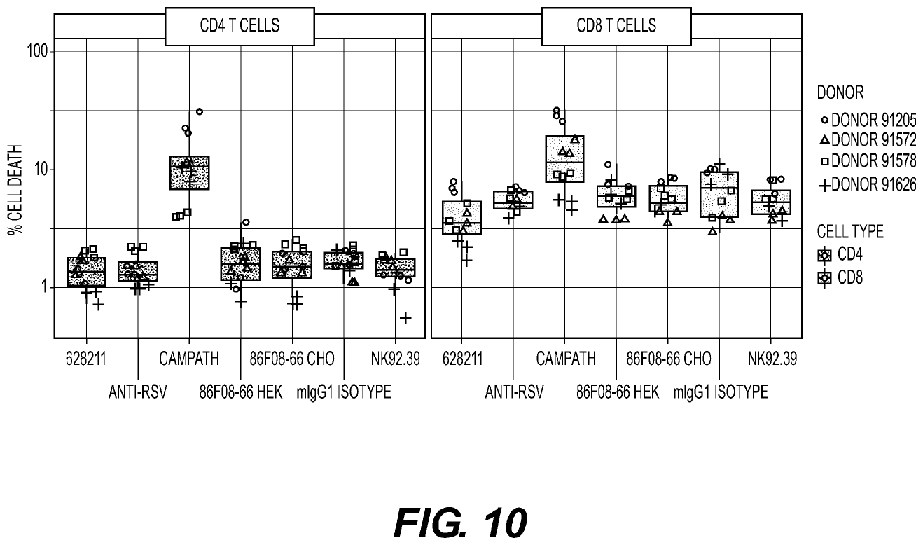

It was important to confirm the findings of the reporter assays by cell killing assays using primary human cells. In fresh human PBMC cultures containing NK cells, there was no evidence of increased cell death of either CD4 or CD8 T cells in the presence of 42Y073-86F08-66 (FIG. 10). In the same experiments both CD4 and CD8 T cells were effectively depleted in the presence of the anti-CD52 antibody (Campath).

The first step of the classical pathway of complement activation is mediated by the binding of complement component C1q to cells that are opsonised with antibodies. The affinity of human complement component C1q binding to 42Y073-86F08-66 was determined using SPR and was higher (KD 94.4 nM) than that of the IgG1 WT isotype control (KD 643.5 nM). The potential for 42Y073-86F08-66 to induce complement-dependent cytotoxicity (CDC) was investigated using primary human T cells as targets.

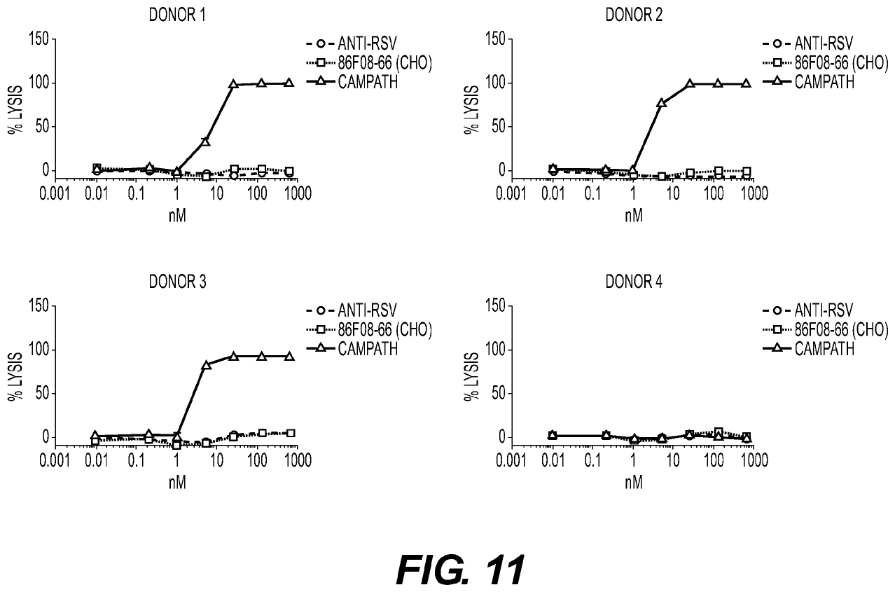

In CDC assays employing human serum as a source of complement, there was no evidence of depletion of either CD4 or CD8 T cells by 42Y073-86F08-66, whereas a control anti-CD52 antibody efficiently mediated depletion of both subsets (FIG. 11). Taken together these data suggest that the risk of ADCC or CDC-mediated depletion of CD96 expressing T cells decorated with 42Y073-86F08-66 is insignificant.

### Example 3

### In Vitro Efficacy and Mechanism Studies of CD96 Binding Proteins

### In Vitro Efficacy of 42Y073-86F08-66 in a Primary Mixed Human PBMC-MLR Assay

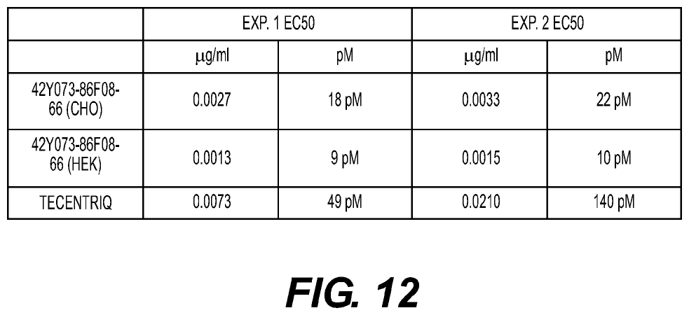

The effect of 42Y073-86F08-66, expressed and purified from CHO cells, and from HEK cells was tested in a primary PBMC assay with no addition of CD155, anti-CD3 or anti-TIGIT. For this assay, PBMCs from 8 different human donors were mixed together and added to the well with different antibodies in solution. After incubation for 3 days, IFNγ in the supernatant was measured by using the homogenous time resolved fluorescence (HTRF) detection method. In this assay, anti-CD3 was not required as PBMCs were activated by the MHC mismatch among different donors. The potency was similar for both CD96 binding proteins (expressed in CHO or HEK cells) (FIG. 12). Both appeared to be more potent than Tecentriq (EC50 49 pM). In a separate repeat of the same experiment, the EC50s were 22 pM, 10 pM and 140 pM for 42Y073-86F08-66 (HEK), 42Y073-86F08-66 (CHO) and Tecentriq respectively (FIG. 12). The cell viability was also measured at the end of 3 days, and only Tecentriq showed some reduced cell viability. None of the anti-CD96 mAbs or isotype controls reduced cell viability, consistent with the observed lack of cell depletion in the ADCC assays.

### CD4⁺ T Cells are the Major Source of 42Y073-86F08-66-Induced IFNγ in the Mixed Human PBMC-MLR Assay

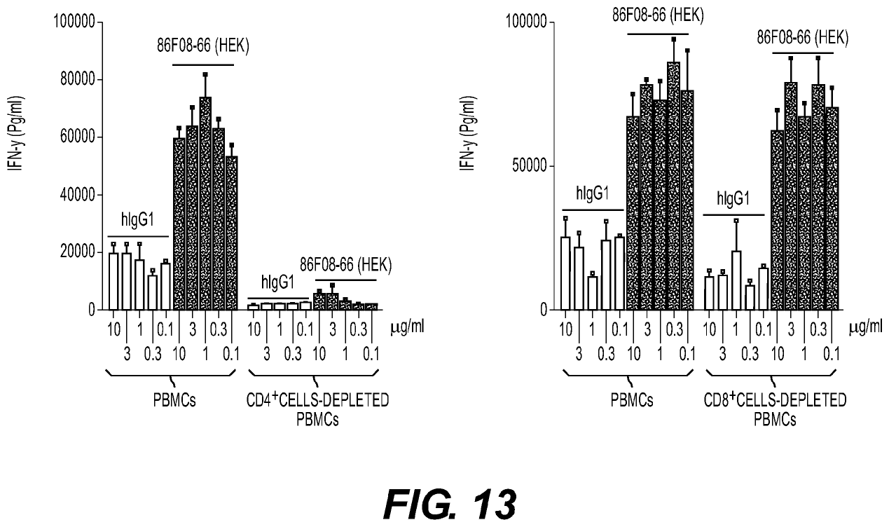

To address the question of what cell sub-populations are responsible for the observed increase of secreted IFNγ in the supernatant upon 42Y073-86F08-66 (HEK) and 42Y073-86F08-66 (CHO) treatment in the PBMC assays, cell depletion studies were carried out. PBMCs from 4 different donors were depleted of CD4⁺ T cells or CD8⁺ T cells separately before mixing together for lymphocyte activation and antibody treatment. Human CD4 or CD8 MicroBeads (Miltenyi) were used to carry out the depletion, and the purity of CD4⁺ or CD8⁺ T cell depletion was validated by flow cytometry. CD4⁺ cells were depleted from PBMCs by 98.5-99.5%, and CD8⁺ cells were depleted from PBMCs by 97.2-100%. Compared with the non-depleted cells, the data obtained from mixed PBMC-MLR assay with T cell depletion indicated that CD4⁺ T cells, but not CD8⁺ T cells were the major subset responsible for the induction of IFNγ by 42Y073-86F08-66 (HEK) in this assay (FIG. 13).

### Flow Cytometry Study to Understand the Mechanism of Action of CD96 Binding Proteins

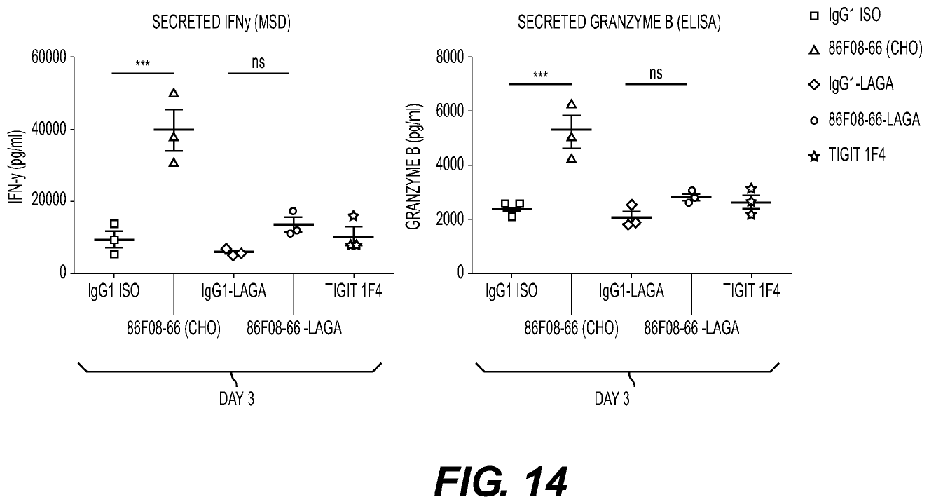

To further understand the mechanism of action for anti-CD96, flow cytometry studies were carried out to study expression changes of cell surface receptors as well as the intracellular cytokines in different cell populations in human PBMCs. The same mixed PBMC-MLR assay system was used. PBMCs from 8 donors were mixed together for cell activation via MHC-mismatch. After anti-CD96 or isotype control antibodies were added, the PBMCs were incubated for 0 day (baseline) or 3 days, and cells were subsequently fixed and various markers were quantified by flow cytometry. To confirm the activity of 42Y073-86F08-66 in this experiment, secreted IFNγ and Granzyme B in the supernatant were also measured by MSD and ELISA after day 3. As observed before, 42Y073-86F08-66 enhanced IFNγ and Granzyme B release (FIG. 14). No increase of secreted IFNγ or Granzyme B was observed with an anti-TIGIT mAb clone 1F4 (Roche, hIgG1-WT).

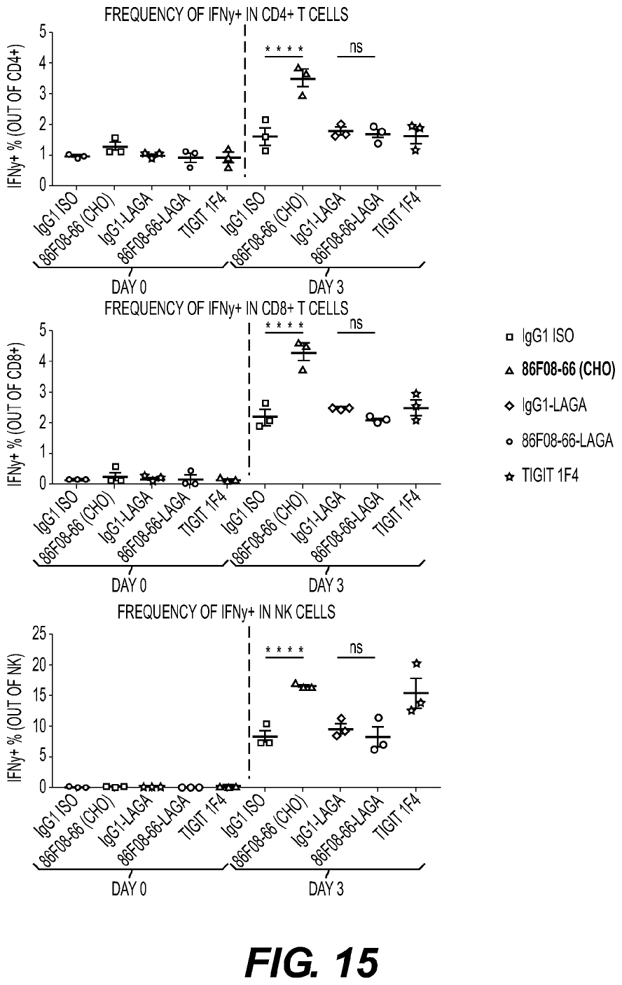

To understand what cell populations contributed to the increased IFNγ by 42Y073-86F08-66, intracellular staining of IFNγ was measured in different cell populations by flow cytometry. Even in the IgG1 isotype control treated group, there was a significant increase in the expression (data not shown) as well as the frequency of IFNγ+CD4+, CD8+ and NK cell subsets at day 3 compared to day 0, indicating lymphocyte activation by mixing the PBMCs from different donors (FIG. 15). Treatment with 42Y073-86F08-66 further significantly increased the frequency of IFNγ⁺ cells in all three immune subsets, namely, CD4⁺, CD8⁺ and NK cells (FIG. 15). Anti-TIGIT antibody (clone 1F4) only increased the frequency of IFNγ+ cells among NK cell population.

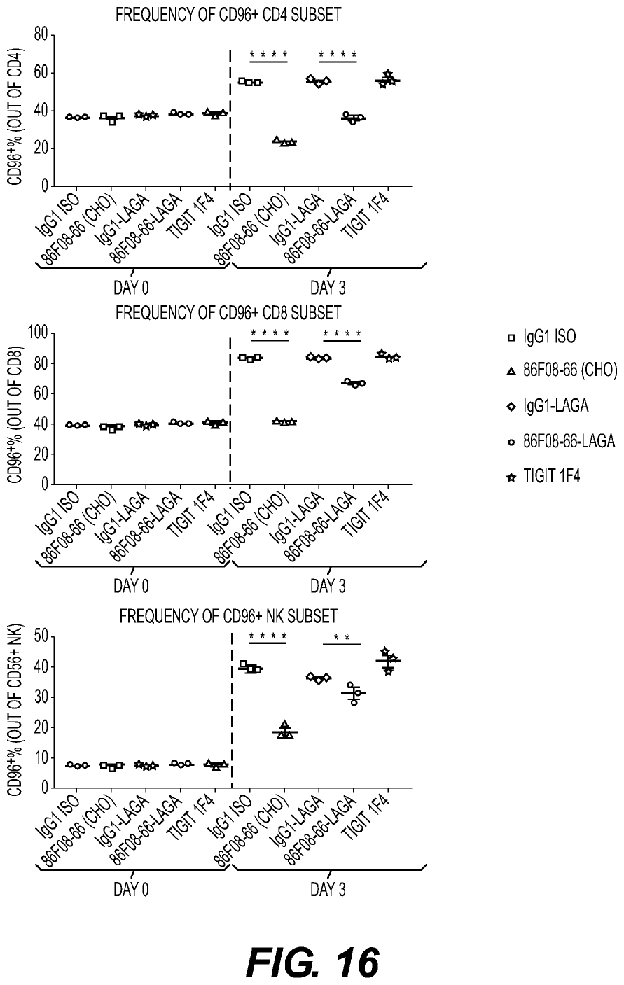

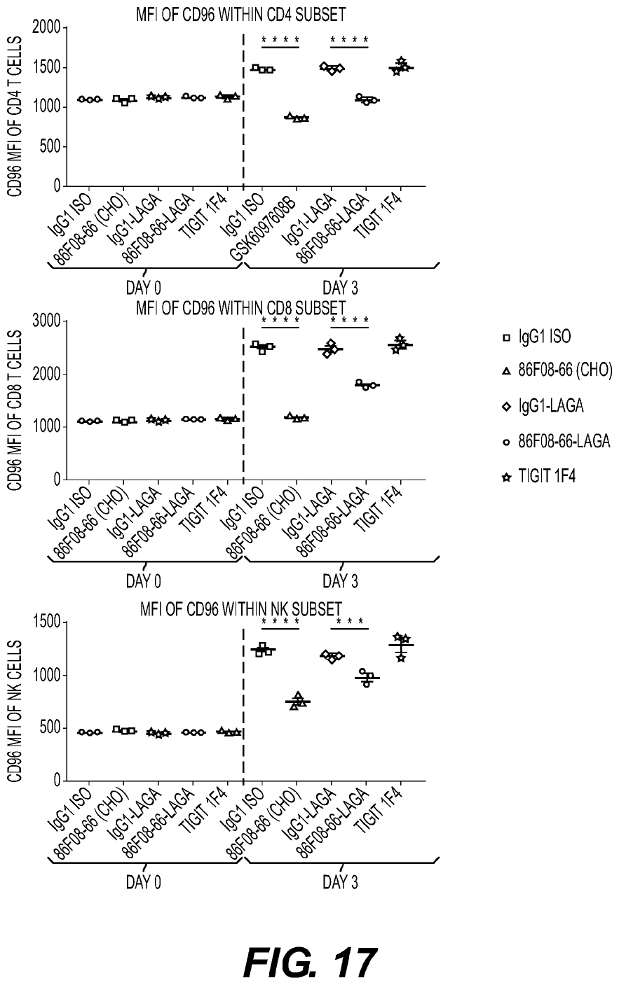

Additionally, CD96 expression was significantly increased in all three cell populations upon cell activation on day 3 comparing to day 0, a profile commonly observed for checkpoint proteins. Anti-CD96 antibody treatment significantly reduced the CD96 MFI and CD96⁺ cell frequencies in all 3 subsets, CD4⁺, CD8⁺ and NK cells, compared to the matched isotype control group. The Fc-disabled 86F08-66-LAGA antibody treatment also led to a reduction in CD96 profile, although the decrease in CD96 expression in the 42Y073-86F08-66 treated group was much more significant (FIGS. 16-17). Anti-TIGIT ab clone 1F4 showed no effect. This appeared reduction of CD96 expression upon 42Y073-86F08-66 treatment could be due to competition between the anti-CD96 detection antibody and the therapeutic Ab which indicates receptor occupancy by the therapeutic antibody, or it could be due to internalization of CD96 receptor upon binding of 42Y073-86F08-66. The detection antibody used in the analysis is a commercial Ab clone 6F9 (BD Biosciences).

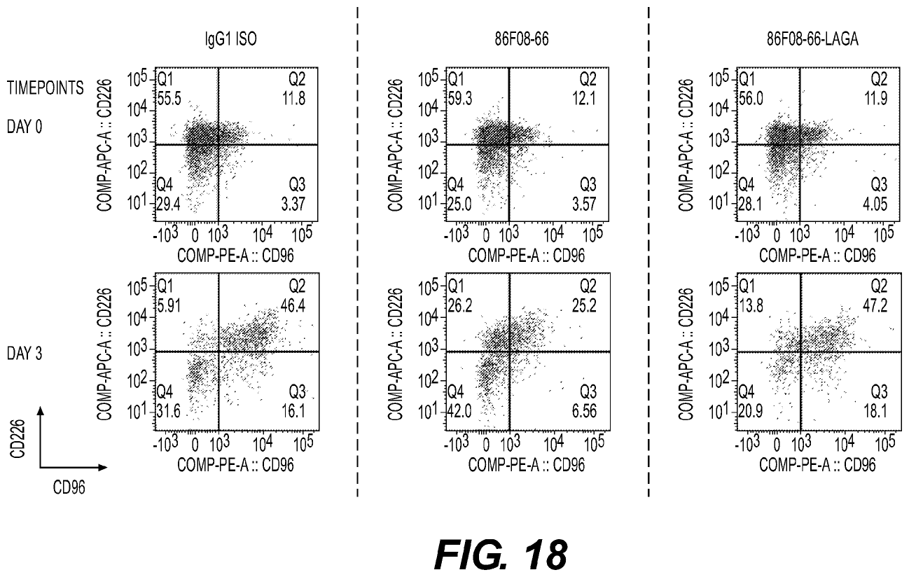

CD226 is one of the major activating receptors in NK cells, however, its role in T cells is not as well established. To understand the mechanism of in more detail, we focused on characterizing the CD226+ population of NK cells in the PBMC-MLR assay. Upregulation of CD96 expression upon cell activation on day 3 increased the frequency of CD226+CD96+NK cells. Treatment with 42Y073-86F08-66 resulted in higher ratio of CD226⁺ single positive vs CD226⁺CD96⁺ double positive NK cells on day 3 (26.2%/25.2%=1) compared to both the isotype control (5.91%/46.4%=0.127), as well as the 86F08-66 IgG1 LAGA Fc-disabled antibody (13.8%/47.2%=0.29), potentially through target occupancy and/or internalization of CD96 (FIG. 18). As CD226 is the activating receptor in the axis and CD96 is a putative checkpoint receptor in the same axis, the CD226⁺ single positive cells may represent more activated NK cells than the CD226⁺CD96⁺ double positive cells.

42Y073-86F08-66 treatment resulted in a higher frequency of IFNγ⁺ cells and GrzB⁺ cells among CD226⁺NK cells on day 3 after 42Y073-86F08-66 treatment. No effect was observed with the Fc-disabled IgG1-LAGA lead antibody (86F08-66-LAGA).

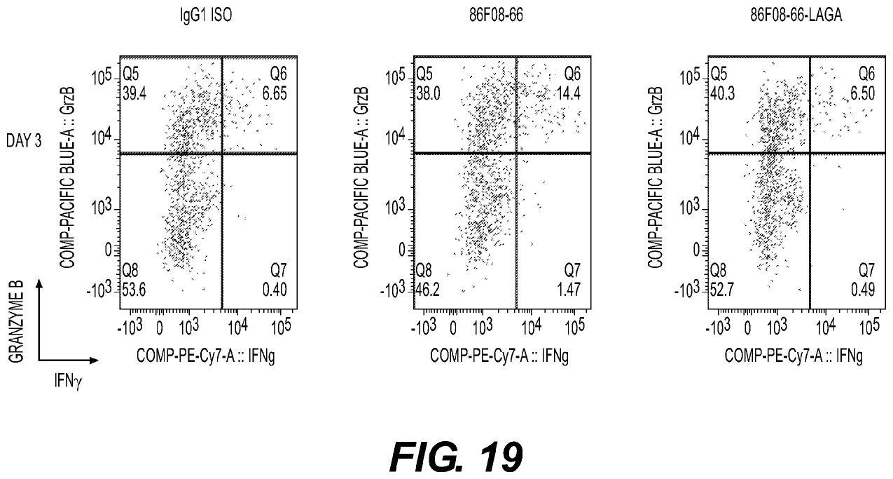

Overall, 42Y073-86F08-66 treatment resulted in more IFNγ⁺GrzB⁺ double positive cells among total NK cells at day 3 comparing to the isotype control Ab or the Fc-disabled IgG1-LAGA lead antibody (86F08-66-LAGA) (FIG. 19).

### CD155-PBMC Assay for Screening of CD96 Binding Proteins

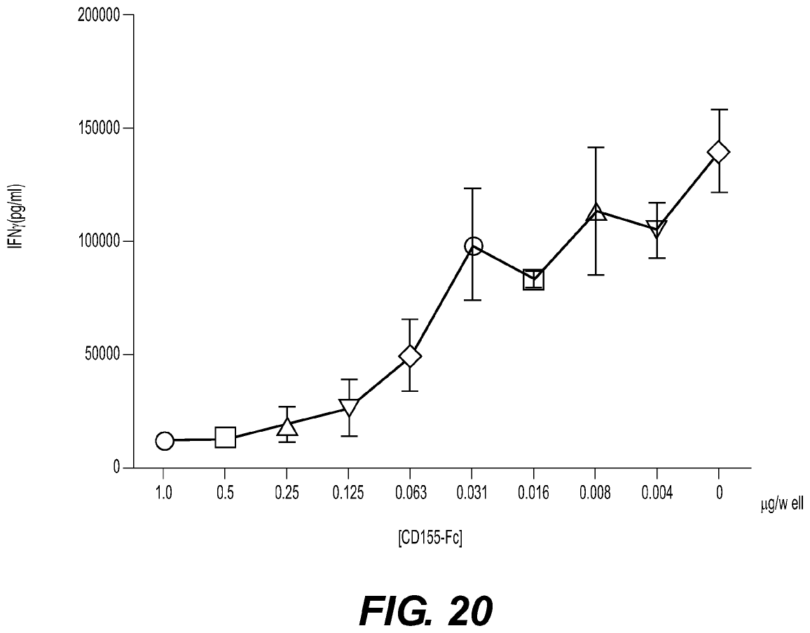

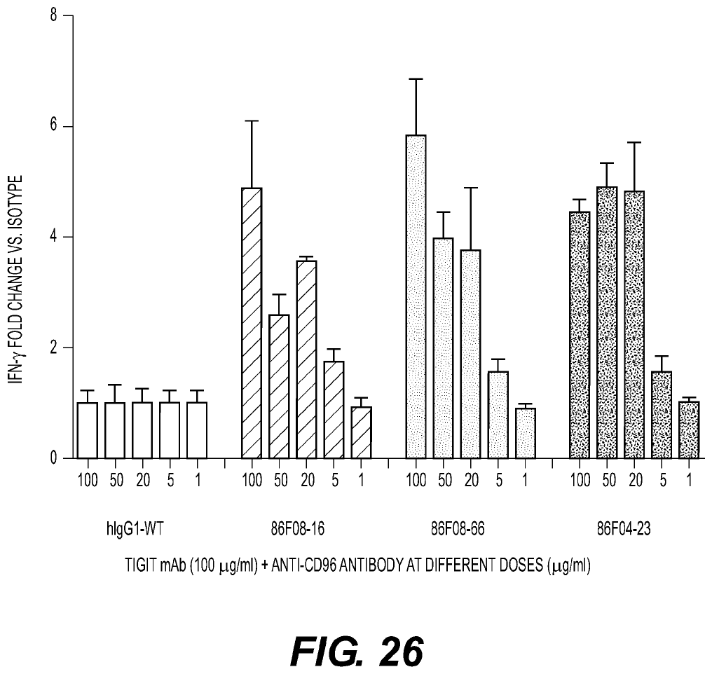

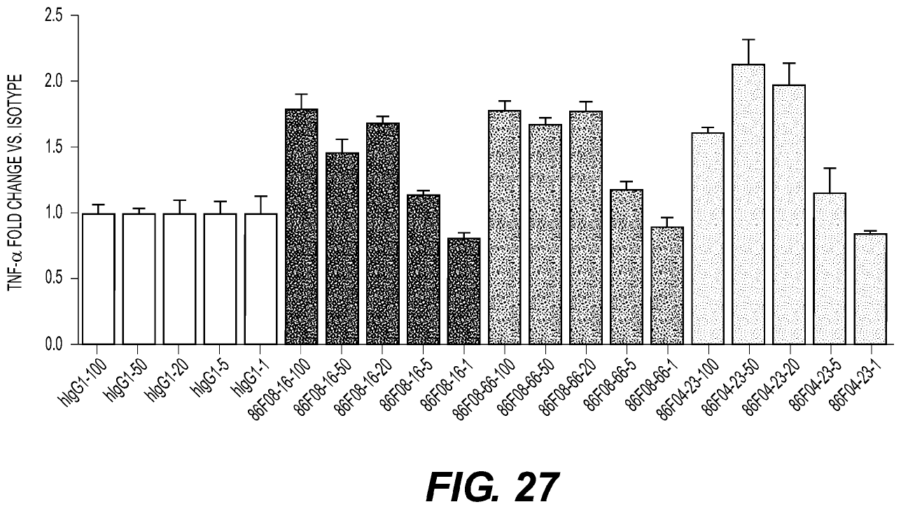

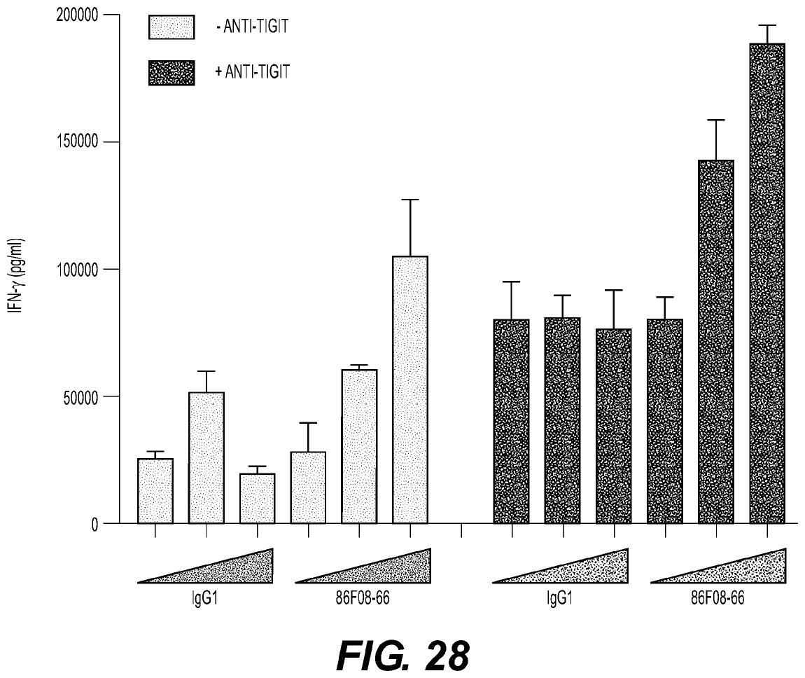

To determine the inhibitory effect of plate-bound CD155-Fc on IFNγ production in human PBMCs, round-bottom 96-well non-TC plate (#351177) were coated with rhCD155-Fc (Cat #9174-CD-050, R&D Systems) at different doses overnight at 4° C. and blocked with AIM-V medium (Cat #12055-091, Thermo Fisher) containing 5% BSA (cat #9576, Sigma) for 30 min at room temperature. PBMCs (Cat #70025, Stemcell Technologies) were pre-treated with CD96 binding proteins, anti-TIGIT mAb (Cat #MAB7898, R&D Systems) or isotype control at room temperature for 10 min were added into wells at 2×10⁵ cells/well in AIM-V medium containing 0.01 μg/ml of anti-CD3 mAb and cultured for 3 days. The supernatants were harvested and stored at −20° C. for measurement of IFNγ and Granzyme B by MSD or ELISA. The inhibitory effect of plate-bound CD155-Fc on IFNγ production in human PMBCs is illustrated in FIG. 20. CD96 binding proteins appear to mitigate CD155-Fc mediated IFNγ inhibition (vs. IgG1-WT isotype control antibody) (FIG. 26). Additional assays were conducted determining the ability of CD96 binding proteins mitigate CD155-Fc mediated TNFα suppression (in the presence of anti-TIGIT mAb) (FIG. 27). Further assays conducted with and without anti-TIGIT mAb indicate that the ability of the CD96 binding proteins to mitigate CD155-Fc inhibition on IFNγ production is apparent both with and without the presence of anti-TIGIT mAb (FIG. 28).

### Activity of CD96 Binding Proteins in Human Tumor Infiltrating Lymphocyte (TIL) Assays

The tumor microenvironment (TME) is immune-suppressive, and tumor infiltrating lymphocytes (TIL) found in the tumors are often immuno-dysfunctional or ‘exhausted’. A primary human TIL assay was developed to evaluate the potential therapeutic effect of 42Y073-86F08-66 in vitro. Fresh, primary resected tumors were mechanically and enzymatically dissociated into single cell suspensions that contain both tumor cells and TILs. The TILs in the cell suspension were mildly activated with a suboptimal dose of soluble anti CD3 (clone HIT3a) and were plated in triplicate into 96 well ultra-low attachment round bottomed spheroid formation plates. The antibodies were then added to the wells, and IFNγ in the supernatant was measured after 6 days. 42Y073-86F08-66 was evaluated in this assay at 3 concentrations (10, 2, and 0.5 μg/mL) alone or in combination with either anti-PD-1 (Keytruda/pembrolizumab) (10 μg/mL) or anti-TIGIT (100 μg/mL). Anti-PD-1 alone was also tested as a positive control for induction of IFNγ above anti-CD3 alone. Appropriate isotype controls were also included.

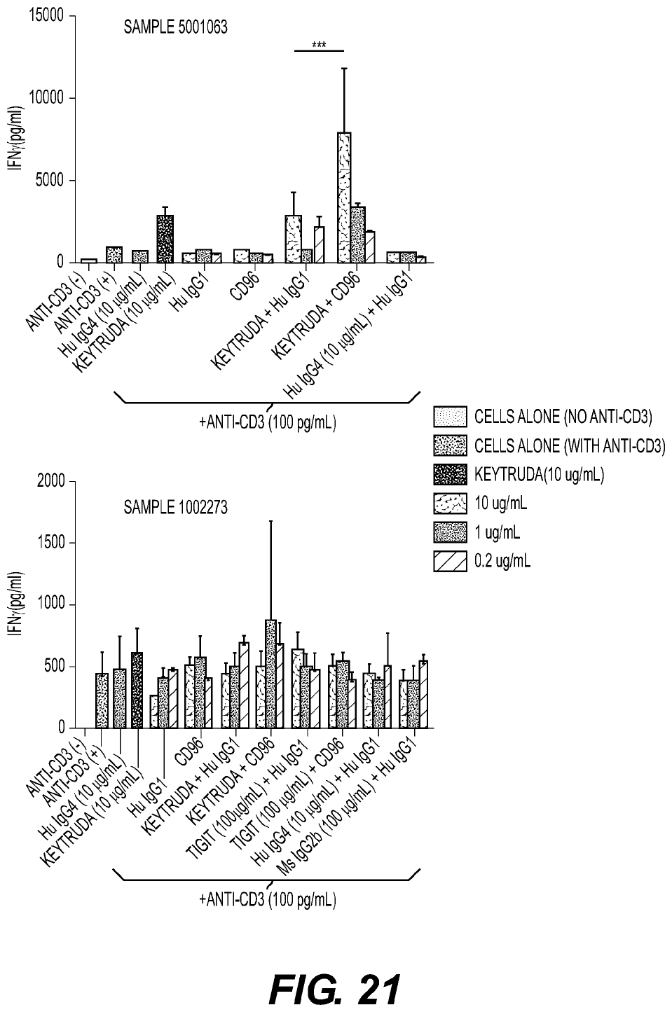

A total of 6 tumors (4 endometrial and 2 renal) were tested in this assay. For the 4 endometrial tumors, anti-PD-1 together with anti-CD3 treatment induced equivalent IFNγ levels as anti-CD3 alone, indicating that the anti-CD3 stimulation may be too high and further technical development may be necessary. In one of the two renal tumors (Sample 5001063), anti-PD-1 treatment augmented (p<0.05 by 1-way Anova) IFNγ production above anti-CD3 alone. Additionally, anti-PD-1+42Y073-86F08-66 (10 μg/mL) combination treatment significantly (p<0.001 by one-way Anova) enhanced IFNγ levels to 7764 pg/mL, higher than the 10 g/mL 42Y073-86F08-66 alone (599 pg/ml), anti-PD-1 alone (2692 pg/mL), or a simple additive effect of the two (FIG. 21). For the second renal tumor (Sample 1002273), anti-PD-1 treatment did not enhance IFNγ production above anti-CD3 stimulation alone. These tumors were not pre-screened for PD-1 axis expression or CD96-axis expression. The complexity of these TIL assays is broadly recognized and variation in response to anti-PD-1 treatment among patient tumors has been observed. This is not surprising given the overall response rate for anti-PD-1 (Keytruda) in unscreened patients is below 25%.

### Example 4

### In Vivo Efficacy and Mechanism Studies

### Bioluminescence Imaging Study of CD96 Binding Protein in a NK Cell Dependent B16F10 Melanoma Lung Colonization Model

NK cells are part of the innate lymphocyte family and play a prominent role in controlling early tumor growth and the spread of metastases through cytotoxic activity and the release of inflammatory cytokines. A frequently used in vivo model of studying NK cell-dependent anti-cancer activity is the B16F10 melanoma lung colonization model. This model was also described in the CD96 cancer related publications (Blake S. J., et al. 2016). We decided to use the same model to study 42Y073-86F08-66 (produced in HEK cells) activity in vivo. To measure the efficacy of anti-CD96 mAb in controlling lung metastasis in real time, and in a more quantitative way, instead of using regular B16F10 melanoma cells, B16F10 cells encoding Red Firefly luciferase (RFluc) were used to allow in vivo imaging of the luciferase signal from the lung indicative of tumor burden.

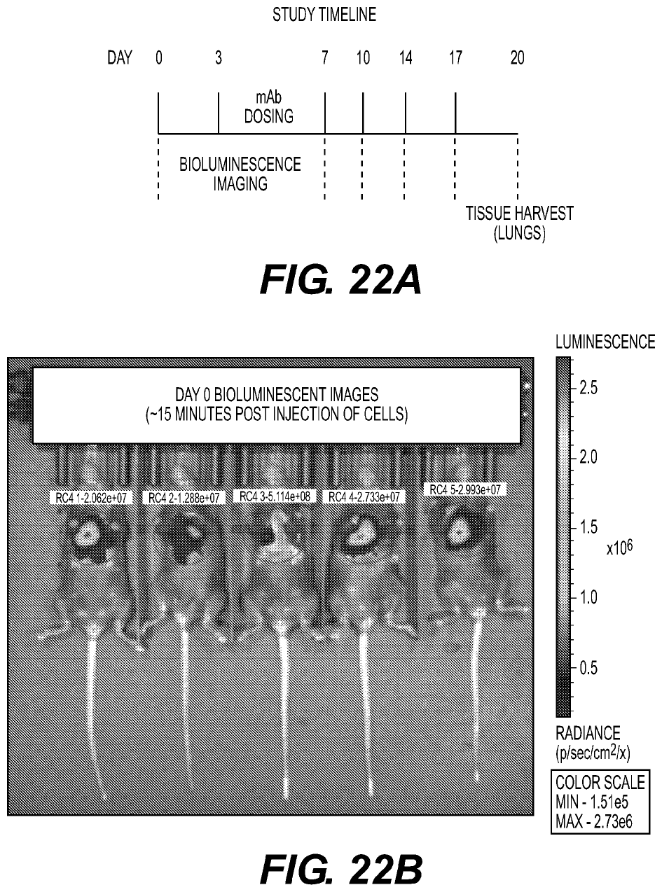

Following the tail vein administration of ˜ 500,000 B16F10 RFluc metastatic melanoma cells, in vivo bioluminescent imaging was performed at Day 0 (˜15 minutes post injection of cells), Day 7, 10, 14, 17, and 20. (Initial cell colonization occurs in the lungs as early as 15 minutes post injection from previous model development studies). Dosing frequency was twice per week (FIG. 22). Following in vivo imaging at Day 20, mice were harvested for ex vivo bioluminescent imaging of the mouse lungs.

To further evaluate the role of specific immune cell types (CD4⁺, CD8⁺, and NK Cells) in this system, CD4⁺ cells, CD8⁺ cells, NK cells or both CD4⁺ and CD8⁺ cells were depleted using an established antibody treatment method. Subsequent flow cytometry analysis confirmed the depletion of NK cells as well as T cells in the CD4⁺/CD8⁺ depletion group.

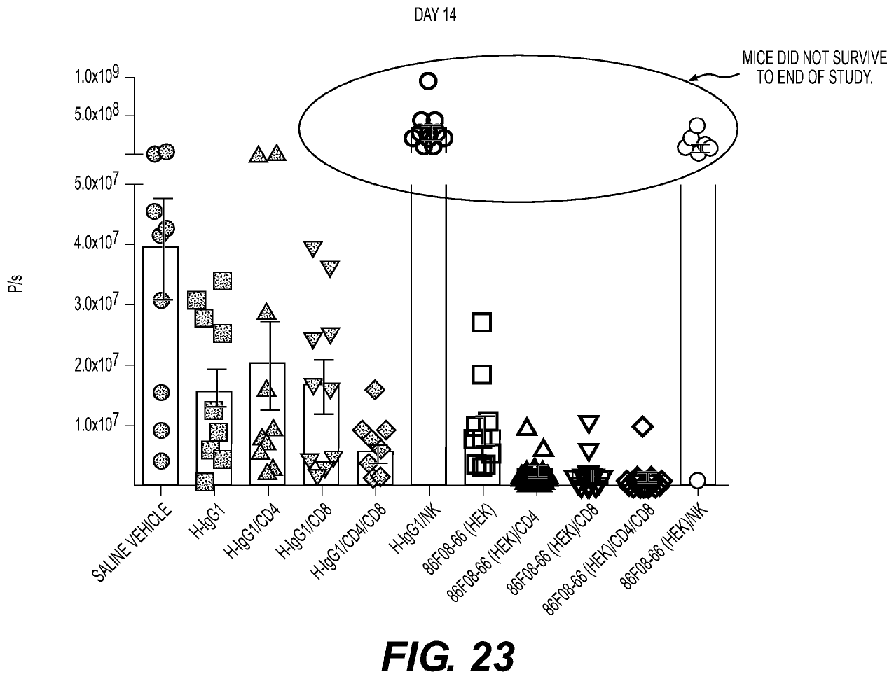

Bioluminescent imaging at Day 14 revealed significantly increased signal in the lungs of NK cell depleted groups (FIG. 23). When there is lower lung signal (p/s) there is less metastases. In fact, 19 out of 20 mice did not survive to the end of the study presumably due to heavy lung tumor burden. This data clearly supports the critical role of NK cells as primarily responsible for suppressing lung metastasis in this model.

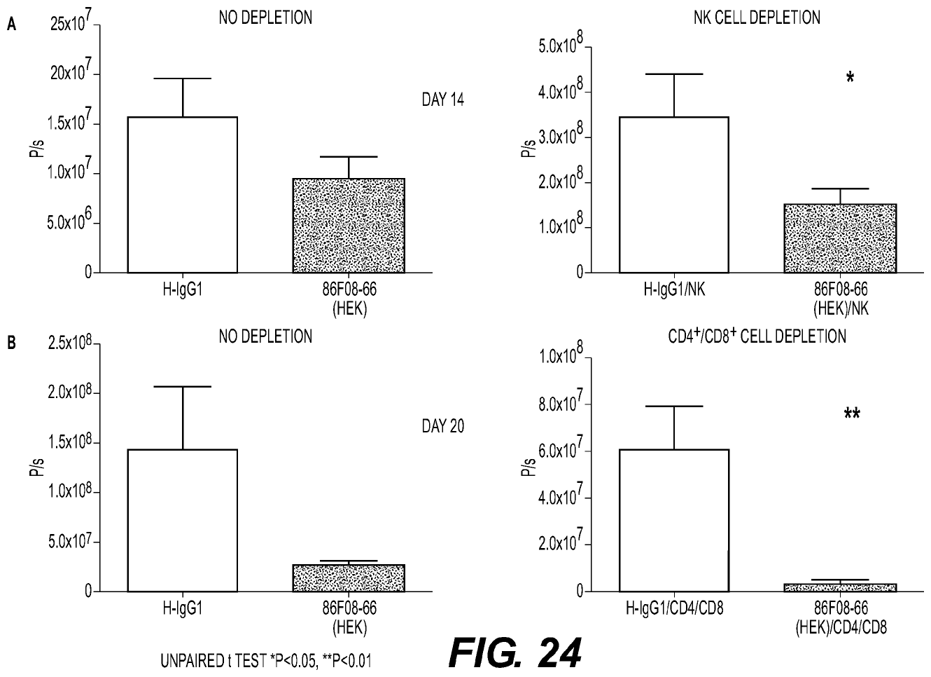

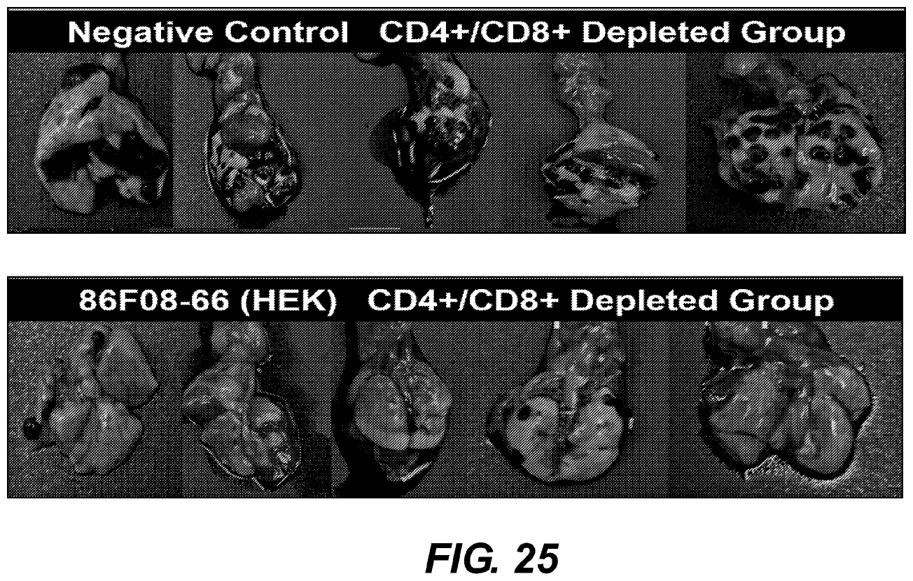

In vivo bioluminescent imaging performed at Day 14 when the NK depleted mice were still alive showed that for NK depleted groups, 42Y073-86F08-66 (produced in HEK cells) treatment significantly reduced lung metastasis (* P<0.05) comparing to isotype control treatment (FIG. 24, A). For the undepleted group, the trend was also observed but not statistically significant. At Day 20, for the undepleted groups, lung signal was decreased in 42Y073-86F08-66 treated group but was not statistically significant (ns). For the T cell depleted groups, 42Y073-86F08-66 significantly reduced (** P<0.01) lung signal compared to isotype control (FIG. 24, B). When there is lower lung signal (p/s) there is less metastases. The digital picture of the lungs after mice were sacrificed on day 20 confirmed the effect of 42Y073-86F08-66 (FIG. 25).

### Example 5

### Efficacy Data and Mechanism Summary

### Evidence of Activity of 42Y073-86F08-66 on T Cells

Human: CD96 is expressed at readily detectable level on both CD4 and CD8 T cells in PBMCs, as well as in tumor microenvironment. In TILs, besides NK cells, CD96 was found on CD8, CD4 Teff cells as well as Tregs. Consistent with its expression pattern, flow cytometry analysis showed that 42Y073-86F08-66 treatment increased the percentage of IFNγ⁺ CD4⁺ and CD8⁺ T cells in the mixed PBMC-MLR assay. Depletion studies showed a dramatic loss of IFNγ release by 42Y073-86F08-66 in the mixed PBMC-MLR assay when CD4 T cells were depleted. However, CD8 depletion was not very impactful in this particular assay.

Mouse: In the in vivo setting, for the B16F10 lung colorization assay, on day 14 after tumor cell injection when NK depleted mice were still alive, when NK cells were depleted, 42Y073-86F08-66 treatment still showed a statistically significant reduction of lung metastasis comparing to isotype control, presumably through activating T cells.

### Evidence of NK Cell Activity

Human: CD96 is expressed at readily detectable level on NK cells in PBMCs, as well as in the tumor microenvironment. In fact, among TIL subpopulations, the highest level of CD96 expression was found on NK cells. CD226 is one of the major activating receptors for NK cells, and strong NK activity target validation data for CD96 was also reported in the literature. In the in vitro setting, intracellular cytokine staining studies using flow cytometry showed increased IFNγ⁺GrzB⁺ NK cells upon 42Y073-86F08-66 treatment in the mixed PBMC-MLR assays.

Mouse: In the in vivo setting, using a well-recognized highly NK cell dependent model, the B16F10 lung colonization model, 42Y073-86F08-66 was able to significantly suppress lung metastasis when both CD4 and CD8 T cells were depleted, strongly suggesting through NK activity.

### Example 6: Anti-CD96 mAb—Nonclinical Toxicology Studies

GSK6097608B (hereafter referred to as GSK6097608 and which is the same as 42Y073-86F08-66) is a monoclonal antibody (mAb) targeting cluster of differentiation (CD)96 that is being developed for the treatment of cancer. Intravenous (IV) dose-range and 4-week repeat-dose toxicity studies with an anti-CD96 mAb have been conducted in cynomolgus monkeys and BALB/c mice. Additionally, a single-dose PK and PD study was conducted in monkeys. An in vitro CRA in human blood samples and a combination CRA with dostarlimab (anti-PD-1 mAb) have been conducted. An assessment of the binding profile was conducted using a human microarray with follow-up confirmatory binding assays. A preliminary immunohistochemistry (IHC) study was performed using selected human and cynomolgus monkey tissues.

BALB/c mouse and cynomolgus monkey were selected as nonclinical species in which to assess the safety profile of the anti-CD96 mAb. The monkey was considered to be an appropriate nonclinical species in which to assess the potential toxicities of the anti-CD96 mAb because the anti-CD96 mAb cross-reacts with similar affinity to human and monkey CD96 receptors. Additional nonclinical safety studies were performed in BALB/c mice based on initial PK and efficacy study results, as well as in vitro binding of the anti-CD96 mAb to murine splenocytes and cytokine production by murine splenocytes prestimulated with CD3/CD28 and incubated with the anti-CD96 mAb.

The anti-CD96 mAb was well tolerated in monkey toxicology studies following 4 weekly doses up to 100 mg/kg/week. CD96 receptor occupancy (RO) or target engagement on CD8+ T cells, CD4+ T cells, and NK cells was maintained throughout all studies. A low incidence of low titer anti-drug antibodies (ADA) was observed that did not affect receptor binding or systemic exposure. There were no changes in the number of circulating cells expressing CD96 and no histopathology findings in tissues, including primary and secondary immune tissues, suggesting the risk of fragment crystallizable (Fc)-dependent depletion of CD96-expressing effector cells is low, the anti-CD96 mAb was tolerated for 4 weeks at 100 mg/kg/week in mice; however, a lower dose of 10 mg/kg/week caused immune-mediated anaphylaxis due to ADA or circulating immune complexes following the third weekly dose administration.

An in vitro CRA evaluating the anti-CD96 mAb alone, dostarlimab alone, and the combination of the anti-CD96 mAb with dostarlimab was conducted using PBMCs isolated from 5 male and 5 female healthy donors. Compared with media controls, there were minimal increases in interleukin (IL)-10, an immunoregulatory anti-inflammatory cytokine, in most donors with each antibody treatment and mild to moderate treatment-related increases in IL-6 and/or tumor necrosis factor alpha (TNF-α) for 1 donor in each of the anti-CD96 mAb-alone and dostarlimab-alone conditions. These levels were less than the anti-CD3 and anti-CD28 positive controls that induced a pan-cytokine response. Overall, there was no augmentation of cytokine response with the combined treatment; however, the 1 donor with increased IL-6 and TNF-α in the anti-CD96 mAb-alone condition was also more sensitive to the combined treatment. The results indicate an overall low risk for cytokine release syndrome (CRS), but there may be individual participant responses for induction of cytokines.

Based on the tolerability and absence of relevant adverse findings, the no observed adverse effect level (NOAEL) was determined to be 100 mg/kg/week (the highest dose tested) in both monkeys (gender-averaged Week 4 mean area under the curve [AUC]0-168h: 580 mg·h/mL [range: 483 to 647 mg·h/mL] and Cmax: 5.54 mg/mL [range: 5.10 to 6.54 mg/mL]) and mice (gender-averaged Week 4 mean AUC0-168h: 319 mg·h/mL and Cmax: 2.98 mg/mL).

### Example 7: First Time in Human Study of Anti-CD96 mAb as a Monotherapy and in Combination with Dostarlimab (Also Known as TSR-042)

GSK6097608B (hereafter referred to as GSK6097608 and which is the same as 42Y073-86F08-66) is a monoclonal antibody (mAb) targeting cluster of differentiation (CD)96 (anti-CD96 mAb) that is being developed for the treatment of cancer. Engagement of CD96 by a related receptor, CD155, functions as an ‘off switch,’ or immune checkpoint, to downregulate immune responses. This CD96 antagonist antibody was developed to block the CD96:CD155 inhibitory axis and increase T cell and natural killer (NK) cell antitumor activity. This first-time-in-human (FTIH) study will evaluate the safety, tolerability, pharmacokinetics (PK), pharmacodynamics (PD), and preliminary clinical activity of GSK6097608 given as monotherapy and in combination with dostarlimab. Dostarlimab (also known as TSR-042) is an investigational humanized mAb of the immunoglobulin (Ig) G4 (IgG4) isotype; it has a high affinity for binding to programmed cell death protein 1 (PD-1), resulting in inhibition of binding to programmed death ligand (PD-L)1 and PD-L2. Based on the observed antitumor activity of other antibodies in the same class, dostarlimab is expected to exhibit clinical activity in a broad spectrum of cancers.

Based on evidence supporting the molecular interplay between pathways and the utility of addressing diverse immune populations, GSK6097608 may not only work in concert with PD-(L)1 (programmed cell death protein 1 and/or programmed death ligand 1) inhibition, but also benefit patients who are refractory to, or have developed resistance to, current T cell-based therapeutics.

Scientific Rationale for Combination with Anti-PD-1

Despite the therapeutic benefit of blocking the immune-checkpoint pathways PD-(L)1 and CTLA-4 across multiple tumor types, most patients do not respond to monotherapy with checkpoint inhibitors, and strategies to increase their activity by combination approaches are being actively explored. The rationale for combining an anti-CD96 mAb (GSK6097608) with an agent designed to block the PD-(L)1 pathway (dostarlimab, anti-PD-1 mAb) is based on evidence supporting the molecular interplay between pathways, the utility of addressing diverse immune populations, and therapeutic complementarity between intervention strategies.

On a mechanistic level, PD-1 signaling has been shown to dephosphorylate the intracellular domain of CD226, attenuating the potential for co-stimulation following CD96-mediated CD155 redirection. Notably, the expression of CD96 is upregulated in melanoma tumor tissue following nivolumab (anti-PD-1) treatment, implicating the CD96 inhibitory axis as a possible adaptive resistance mechanism to PD-1 blockade. Reciprocal upregulation of pathway components may also be observed with CD96 blockade, as GSK6097608-mediated induction of interferon gamma (IFNγ) has the potential to upregulate PD-L1 expression. Collectively, these observations suggest that co-blockade of CD96 and PD-(L)1 may be necessary to enable effective antitumor immune responses.

Clinical Experience with Dostarlimab

Dostarlimab is currently being developed as a monotherapy for patients with recurrent or advanced solid tumors, including endometrial cancer (microsatellite stable and microsatellite instability-high [MSI-H] tumors), non-small-cell lung cancer (NSCLC), and nonendometrial MSI-H solid tumors and polymerase E-mutated cancer. In addition, dostarlimab is being developed as a combination therapy with other therapeutic agents for patients with advanced solid tumors (including melanoma, NSCLC, and colorectal cancer) or advanced or metastatic cancer (including endothelial ovarian cancer, triple-negative breast cancer, and urothelial carcinoma).

As of 21 Jan. 2019, there were 4 ongoing Phase 1 studies, 2 ongoing Phase 2 studies, and 1 ongoing Phase 3 study with dostarlimab.

The safety and tolerability of dostarlimab have been evaluated in over 627 participants with advanced cancer who have received at least 1 dose of dostarlimab. Of the 335 participants treated with dostarlimab monotherapy in the FTIH Study 4010-01-001 (GARNET), 93.7% reported at least 1 treatment-emergent adverse event (TEAE), with events of fatigue, nausea, and diarrhea being the most frequently reported. Study intervention-related TEAEs≥Grade 3 were reported in 36 participants (10.7%). The majority of study intervention-related TEAEs≥Grade 3 occurred in only 2 participants each, with the exception of fatigue (6 participants), alanine aminotransferase (ALT) increased (4 participants), anemia (4 participants), aspartate aminotransferase (AST) increased (3 participants), and lipase increased (3 participants). Serious adverse events (SAEs) occurred in 106 participants (31.6%); in 21 of these participants, these SAEs were considered study-intervention related. All study intervention-related SAEs occurred in 1 participant each, with the exception of pneumonitis (4 participants), dyspnea (2 participants), pyrexia (2 participants), and rash maculopapular (2 participants). Twenty-three participants (6.9%) who received dostarlimab monotherapy experienced at least 1 immune-related adverse event (irAE) with severity of ≥Grade 3; for 18 of 23 participants with ≥Grade 3 irAEs, the adverse event (AE) was assessed as study-intervention related by investigators. The majority of ≥Grade 3 irAEs were reported in ≤2 participants each. The irAEs ≥Grade 3 reported in >2 participants were AST increased (4 participants), ALT increased (4 participants), lipase increased (4 participants), and rash (3 participants).

Of the 292 participants treated with dostarlimab in combination with other therapeutic agents, 94.5% reported at least 1 TEAE, with events of fatigue, nausea, and dyspnea being the most frequently reported. SAEs occurred in 108 participants (37.0%); in 20 of these 108 participants, the SAEs were related to the study intervention.

Based on the safety data from human experience and the available nonclinical pharmacology and toxicology information, dostarlimab has demonstrated an acceptable clinical and nonclinical safety profile that appears to be consistent with the safety experience of approved mAb PD-1 inhibitors, pembrolizumab and nivolumab.

Based on the observed antitumor activity of other antibodies in the same class, dostarlimab is expected to exhibit clinical activity in a broad spectrum of cancers. Preliminary efficacy data from 15 participants with MSI-H endometrial cancer and 24 participants with NSCLC who had at least 1 tumor assessment were presented at the April 2018 annual meeting of the American Association for Cancer Research. Responses were assessed by investigators using immune-related Response Evaluation Criteria in Solid Tumors (irRECIST). Among the 15 participants with MSI-H endometrial cancer, the overall response rate was 47% and consisted of all partial responses (PRs); 20% had stable disease (SD) and 33% had disease progression. Among the 24 participants with NSCLC, the ORR was 29% and consisted of all PRs; 42% had SD, and 17% had disease progression. Thus, preliminary efficacy data from participants with NSCLC treated with dostarlimab appear to be comparable to the reported efficacy of other PD-(L)1 inhibitors, such as pembrolizumab, in participants with advanced and recurrent cancer (ORR: 19%) [Herbst, 2016].

### Objectives and Endpoints

This FTIH, open-label, dose-escalation study will assess the safety, tolerability, PK, PD, and preliminary clinical activity of GSK6097608 in participants with locally advanced, recurrent, or metastatic solid tumors as monotherapy (Arm A) or in combination with dostarlimab (Arm B); the study will be used to define the recommended Phase 2 dose (RP2D).

TABLE 10
Study summary:

| | |
|---|---|
| Objectives | Endpoints |

Primary

| | |
|---|---|
| To determine the safety, tolerability, and | Incidence of DLTs |
| the RP2D of GSK6097608 administered | Incidence, duration, and severity of AEs |
| IV as monotherapy (Arm A) or in | and SAEs |
| combination with dostarlimab (Arm B) to | |
| participants with advanced or recurrent | |
| solid tumors | |

Secondary

| | |
|---|---|
| To further characterize the safety and | Changes in safety assessments (eg, |
| tolerability of GSK6097608 administered | laboratory parameters, vital signs, |
| IV as monotherapy (Arm A) or in | cardiac parameters) |
| combination with dostarlimab (Arm B) to | Dose modifications (eg, dose reductions |
| participants with advanced or recurrent | or delays) |
| solid tumors | Withdrawals due to AEs |
| To evaluate the antitumor activity of | ORR based on RECIST 1.1 criteria |
| GSK6097608 as monotherapy (Arm A) or | |
| in combination with dostarlimab (Arm B) | |
| in participants with advanced or recurrent | |
| solid tumors | |
| To evaluate immunogenicity to | Incidence and titers of ADA to |
| GSK6097608 as monotherapy (Arm A) | GSK6097608 (Arm A and Arm B) and |
| and in combination with dostarlimab | dostarlimab (Arm B) |
| (Arm B) | |
| To characterize the PK properties of | GSK6097608 (Arms A and B): plasma |
| GSK6097608 as monotherapy (Arm A) | concentrations, PK parameters such as |
| and in combination with dostarlimab | Cmax, Cmin, AUC, t½, as data permit |
| (Arm B) | Dostarlimab (Arm B): plasma |
| | concentrations, PK parameters such as |
| | Cmax, Cmin, AUC, t½, as data permit |

Exploratory

| | |
|---|---|
| To further evaluate the clinical activity of | ORR based on iRECIST criteria |
| GSK6097608 | DCR, TTR, DoR, and TTP based on |
| | RECIST 1.1 and iRECIST, as data permit |
| To evaluate the PD effect of | PD assessment of blood and tumor |
| GSK6097608 in blood and tumor when | biomarkers, which may include the CD96 |
| administered as monotherapy (Arm A) or | axis, immune cell phenotypes, gene |
| in combination with dostarlimab (Arm B) | expression (RNA), genomic DNA, T-cell |
| | or B-cell receptor sequences, cell-free |
| | tumor nucleic acid, and various |
| | measures of immune function |
| To evaluate the exposure-response | Relationships between parameters of PK, |
| relationships for GSK6097608 for PD, | clinical activity, safety endpoints, and PD |
| clinical activity, and safety | markers, which may include |
| | PK (plasma concentrations, PK |
| | parameters such as Cmax, Cmin, |
| | AUC, t½, as data permit) |
| | PD biomarkers (eg, immune cell |
| | phenotypes, measures of immune |
| | function) |
| | ORR based on RECIST 1.1 criteria |
| | Safety (eg, laboratory parameters, |
| | AEs, and SAEs) |
| To explore the association between | Correlation between antitumor activity |
| GSK6097608 antitumor activity and | and baseline biomarkers, which may |
| biomarkers in tumor and blood as | include the CD96 axis and other immune |
| monotherapy (Arm A) and in combination | phenotyping markers by IHC or |
| with dostarlimab (Arm B) | transcription (RNA) in tumor, soluble |
| | serum biomarkers, and germline or tumor |
| | DNA characteristics |
| Pharmacogenetics: To investigate the | Germline genetic evaluations may be |
| relationship between genetic variations in | conducted for: |
| the germline DNA and response to | Response, including |
| therapy | GSK6097608 alone or in |
| | combination with any |
| | concomitant medicines |
| | Disease susceptibility, severity, |
| | progression, and related |
| | conditions |
| To explore the gut microbiome | Sequencing of the microbiome from stool |
| composition and relationship to treatment | samples. Analysis of the data to identify |
| response | potential selection biomarkers for |
| | participant enrichment |
| | Correlation between antibiotic/probiotic |
| | use prior to treatment and antitumor |
| | activity |

Abbreviations: ADA = anti-drug antibodies; AE = adverse event; AUC = area under the curve; Cmax = maximum concentration; Cmin = minimum concentration; DCR = disease control rate; DLT = dose-limiting toxicity; DNA = deoxyribonucleic acid; DoR = duration of response; IHC = immunohistochemistry; iRECIST = modified Response Evaluation Criteria in Solid Tumors, version 1.1 for immune-based therapeutics; IV = intravenous(ly); ORR = overall response rate; PD = pharmacodynamic(s); PK = pharmacokinetic(s); RNA = ribonucleic acid; RECIST 1.1 = Response Evaluation Criteria in Solid Tumors, version 1.1; RP2D = recommended Phase 2 dose; SAE = serious adverse event; t½ = half-life; TTP = time to progression; TTR = time to response.

### Overall Design

This is a FTTH, open-label, nonrandomized, multicenter study designed to investigate the safety, tolerability, PK, PD, and preliminary clinical activity of escalating doses of GSK6097608 administered IV as monotherapy (Arm A) or in combination with dostarilimab (Arm B) to participants with locally advanced, recurrent, or metastatic solid tumors.

GSK6097608 administered IV every 3 weeks (Q3W) will be evaluated as monotherapy in escalating doses (Arm A) under guidance from a Dose Escalation Committee (DEC). To further evaluate the PK and PD, additional participants will be enrolled in a PK/PD cohort at the RP2D (up to 15 participants) in the following tumor types: non-small-cell lung cancer (NSCLC), head and neck squamous cell carcinoma (HNSCC), or other tumor types defined based on emerging nonclinical and/or clinical data. Additional participants may also be enrolled in PK/PD cohorts at previously cleared dose levels (up to 15 participants per cohort) for dose exploration. These additional participants will contribute to the assessment of safety and preliminary anticancer activity, as well as the overall PK/PD data profile.

Once a dose of GSK6097608 has been identified that is both tolerable and has adequate drug exposure based on PK data, enrollment in the combination arm (Arm B) may begin. In Arm B, escalating doses of GSK6097608 will be evaluated with a fixed dosing regimen of dostarlimab. To further evaluate the PK and PD, additional participants will be enrolled in a PK/PD cohort at the RP2D (up to 15 participants) in the following tumor types: NSCLC, HNSCC, or other tumor types defined based on emerging nonclinical and/or clinical data. In addition to the cohort treated at the RP2D, participants may also be enrolled in PK/PD cohorts at previously cleared dose levels (up to 15 participants per cohort) for dose exploration.

These additional participants will contribute to the assessment of safety and preliminary anticancer activity, as well as the overall PK/PD data profile.

Assessment of disease status at Screening and during study intervention visits will be performed by the investigator in accordance with Response Evaluation Criteria in Solid Tumors, version 1.1 (RECIST 1.1) and a modified RECIST for immune-based therapeutics (iRECIST). A decision to discontinue treatment due to disease progression will be based upon iRECIST; however, some secondary and exploratory anticancer activity analyses will be based on RECIST 1.1. Scans will be collected centrally and stored to allow for the option of central review.

### Analysis

After each dosing cohort, the Neuenschwander Continual Reassessment Method (N-CRM) will be used to guide monotherapy and combination-therapy dose escalation. Dose-escalation decisions will be based primarily on dose-limiting toxicities (DLTs); however, the totality of clinical safety assessments, PK, and PD data will be considered. No formal statistical hypotheses will be tested, and analyses will be descriptive.

This is a 2-arm, open-label, intervention study.

### Number of Participants

Approximately 100 participants will be enrolled to receive study intervention. The total number of participants to be enrolled is an estimate and will depend on the number needed to adequately characterize the DLT profile and determine the RP2D.

### Intervention Groups and Duration

In Arm A (monotherapy), GSK6097608 will be administered IV over approximately 30 minutes to participants under medical supervision of an investigator or appropriately qualified designee.

In Arm B (combination therapy), GSK6097608 will be administered IV first. Dostarlimab will then be administered IV over 30 minutes following the end of the GSK6097608 infusion. Both study interventions will be administered under medical supervision of an investigator or designee.

The study comprises 3 periods: Screening (assessments up to 28 days prior to first dose), Treatment (until disease progression, unacceptable toxicity, death, or withdrawal of consent), and Treatment Discontinuation and Follow-Up (90 days). The total duration of study participation begins with the signing of the informed consent form (ICF) and continues through the final protocol-defined follow-up assessment period. The maximum duration of study participation will depend on the participant's duration of study intervention, with an approximate duration of up to 2 years.

### Tumor Imaging and Disease Assessments

Lesion assessment method and timing, evaluation of disease, disease progression, and response criteria will be conducted according to Response Evaluation Criteria in Solid Tumors, version 1.1 (RECIST 1.1). RECIST 1.1 will be used in the assessment of disease burden (target and nontarget lesions determination) at Screening and as the primary measure of tumor response endpoints. iRECIST will be used by investigators to assess tumor response and progression and make treatment decisions.

### Example 8: Treatment of Cancer with a Dual Combination of a TIGIT Antagonist and a PVRIG Antagonist

A patient being treated for cancer undergoes a treatment regimen in which a first therapy is administered subcutaneously and a second therapy is administered intravenously. More specifically, the patient is first administered a PD-1 antagonist by subcutaneous injection. Subsequently, the patient is administered a combination of a TIGIT antagonist and a PVRIG antagonist by intravenous injection.

A suitable PD-1 antagonist for the treatment regimen comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156. Another suitable PD-1 antagonist comprises VH comprising SEQ ID NO: 158 and VL comprising SEQ ID NO: 159. Yet another suitable PD-1 antagonist comprises HC comprising SEQ ID NO: 160 and LC comprising SEQ ID NO: 161. Dostarlimab is still another suitable PD-1 antagonist.

A suitable TIGIT antagonist for the treatment regimen comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. Another suitable TIGIT antagonist comprises VH comprising SEQ ID NO: 329 and VL comprising SEQ ID NO: 330. Vibostolimab is still another suitable TIGIT antagonist. Yet another suitable TIGIT antagonist comprises an anti-TIGIT antibody comprising a heavy chain and a light chain variable sequence as shown in Table X.

A suitable PVRIG antagonist for the treatment regimen comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. Another suitable PVRIG antagonist comprises VH comprising SEQ ID NO: 279 and VL comprising SEQ ID NO: 280.

### Example 9: Treatment of Cancer with a Dual Combination of a PD-1 Antagonist and a TIGIT Antagonist Followed by a Dual Combination of a Different PD-1 Antagonist and a CD96 Antagonist

A patient being treated for cancer undergoes a treatment regimen in which the patient receives a first therapeutic treatment for a period of time and then is switched to a second, different therapeutic treatment for a period of time. More specifically, the first therapeutic treatment includes administration of a first PD-1 antagonist in combination with a TIGIT antagonist for a period of time, after which administration of the first PD-1 antagonist and the TIGIT antagonist is discontinued. Subsequently, a second therapeutic treatment is initiated that includes administration of a second PD-1 antagonist, different from the first PD-1 antagonist, in combination with a CD96 antagonist.

In one example, the patient is administered a combination of a first PD-1 antagonist such as pembrolizumab and a TIGIT antagonist such as vibostolimab for a period of time, after which the treatment with the first PD-1 antagonist and the TIGIT antagonist is discontinued. Subsequently, the patient is administered a combination of a second, different PD-1 antagonist such as dostarlimab and a CD96 antagonist for a period of time.

In another example, the patient is administered a combination of a first PD-1 antagonist such as dostarlimab and a CD96 antagonist for a period of time, after which the treatment with the first PD-1 antagonist and the CD96 antagonist is discontinued. Subsequently, the patient is administered a combination of a second, different PD-1 antagonist such as pembrolizumab and a TIGIT antagonist such as vibostolimab for a period of time.

Another suitable PD-1 antagonist for the treatment regimen comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156. Still another suitable PD-1 antagonist comprises VH comprising SEQ ID NO: 158 and VL comprising SEQ ID NO: 159. Yet another suitable PD-1 antagonist comprises HC comprising SEQ ID NO: 160 and LC comprising SEQ ID NO: 161.

Another suitable TIGIT antagonist for the treatment regimen comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. Still another suitable TIGIT antagonist comprises VH comprising SEQ ID NO: 329 and VL comprising SEQ ID NO: 330. Another suitable TIGIT antagonist for the treatment regimen comprises an anti-TIGIT antibody comprising a heavy chain and a light chain variable sequence as shown in Table X.

A suitable CD96 antagonist comprises CDRH1 comprising SEQ ID NO: 115, CDRH2 comprising SEQ ID NO: 145, CDRH3 comprising SEQ ID NO: 147, CDRL1 comprising SEQ ID NO: 97, CDRL2 comprising SEQ ID NO: 99, and CDRL3 comprising SEQ ID NO: 101. Another suitable CD96 antagonist comprises VH comprising SEQ ID NO: 86 and VL comprising SEQ ID NO: 85. Yet another suitable CD96 antagonist comprises HC comprising SEQ ID NO: 165 and LC comprising SEQ ID NO: 166.

### Example 10: Treatment of Cancer with a Triple Combination of a PD-1 Antagonist, a TIGIT Antagonist, and a PVRIG Antagonist

A patient being treated for cancer undergoes a treatment regimen using a combination of three antagonists. More specifically, the patient is administered a combination of a PD-1 antagonist, a TIGIT antagonist, and a PVRIG antagonist. The PD-1 antagonist, TIGIT antagonist, and PVRIG antagonist may be administered sequentially. Alternatively, the PD-1 antagonist, TIGIT antagonist, and PVRIG antagonist may be administered concurrently.

A suitable PD-1 antagonist for the treatment regimen comprises CDRH1 comprising SEQ ID NO: 151, CDRH2 comprising SEQ ID NO: 152, CDRH3 comprising SEQ ID NO: 153, CDRL1 comprising SEQ ID NO: 154, CDRL2 comprising SEQ ID NO: 155, and CDRL3 comprising SEQ ID NO: 156. Another suitable PD-1 antagonist comprises VH comprising SEQ ID NO: 158 and VL comprising SEQ ID NO: 159. Yet another suitable PD-1 antagonist comprises HC comprising SEQ ID NO: 160 and LC comprising SEQ ID NO: 161. Dostarlimab is still another suitable PD-1 antagonist.

A suitable TIGIT antagonist for the treatment regimen comprises CDRH1 comprising SEQ ID NO: 334, CDRH2 comprising SEQ ID NO: 335, CDRH3 comprising SEQ ID NO: 336, CDRL1 comprising SEQ ID NO: 331, CDRL2 comprising SEQ ID NO: 332, and CDRL3 comprising SEQ ID NO: 333. Another suitable TIGIT antagonist comprises VH comprising SEQ ID NO: 329 and VL comprising SEQ ID NO: 330. Vibostolimab is still another suitable TIGIT antagonist. Another suitable TIGIT antagonist for the treatment regimen comprises an anti-TIGIT antibody comprising a heavy chain and a light chain variable sequence as shown in Table X.

A suitable PVRIG antagonist for the treatment regimen comprises CDRH1 comprising SEQ ID NO: 223, CDRH2 comprising SEQ ID NO: 224, CDRH3 comprising SEQ ID NO: 225, CDRL1 comprising SEQ ID NO: 226, CDRL2 comprising SEQ ID NO: 227, and CDRL3 comprising SEQ ID NO: 228. Another suitable PVRIG antagonist comprises VH comprising SEQ ID NO: 279 and VL comprising SEQ ID NO: 280.

### Example 11: Dissecting the CD226 Immune Axis in Cancer Using CyTOF-Based High-Dimensional Immunophenotyping

Expression of CD226 axis members (CD96, PVRIG, TIGIT, CD226) was analyzed across immune subsets in various human tumor indications using high-parameter CyTOF (mass cytometry by time of flight) characterization. The results indicated a notable divergence in expression profiles between the CD226 axis members across tumor indications. For example, TIGIT expression was found to be highest on CD4+ regulatory T (Treg) cells, where its expression correlated strongly with ICOS, FoxP3, CD25, and CCR8. By contrast, CD96 and PVRIG exhibited broad expression across T and NK cell subsets. Despite relatively consistent expression profiles of certain CD226 axis members (i.e., TIGIT on Treg cells) across tumors, several cell subsets/clusters unique to specific indications were also identified.

Materials and Methods:

Tumor samples (104) derived from a variety of cancer indications were immunophenotyped using a bespoke 46-parameter CyTOF panel. Using a suite of high dimensional analytical tools, including FlowSOM, UMAP, and tSNE, we revealed distinct expression profiles for each receptor. Of note, this study encompassed 6 separate experiments, and a reference control (healthy PBMC) was used to evaluate batch effects across experiments.

All material was purchased from Discovery Life Sciences (DLS, Huntsville, AL) as pre-processed/dissociated cryopreserved samples. All samples were treatment-naïve. On the day of staining, samples were thawed and directly stained, ex vivo. To prevent significant material loss, samples were acquired on the CyTOF within 48 hours of staining. For some patients, matched peripheral blood mononuclear cells (PBMCs) were also collected and analyzed (data not shown here). Tumor types analyzed are shown in Table 11 below.

TABLE 11
Cancer Patient Samples

| Indication | Tumor Information |
|---|---|
| Bladder (n = 5) | Primary tumor |
| Breast (n = 5) | Primary tumor |
| ccRCC (n = 13) | Primary tumor |
| Cervical (n = 3) | Primary tumor |
| CRC (n = 5) | Primary tumor |
| EC (n = 2) | Primary tumor |
| Gastric (n = 10) | Primary tumor |
| HNSCC (n = 9) | Primary tumor |
| Liver (n = 5) | Primary tumor |
| Lung SCC (n = 7) | Primary tumor |
| Melanoma (n = 6) | Metastatic lymph node |
| NSCLC (n = 24) | Primary tumor |
| Ovarian (n = 10) | Primary tumor |

Cell Thawing

Frozen cells were thawed in a 37° C. water bath, slowly added to 1 mL pre-warmed AIM-V medium, and another 9 mL AIM-V medium were added to the cells. Cells were then centrifuged at 300×g for 5 minutes at room temperature, resuspended in 1 mL AIM-V medium and filtered through a 70 μM filter. The washing step was repeated with 10 ml AIM-V medium. Cells were counted at 1:10 dilution using a Vi-Cell cell counter. According to cell count, 2-4e6 cells in 200 μL Cell Staining Buffer were added to the wells of a round-bottom polystyrene 96-well plate.

Cell Staining

All centrifugation steps prior to fixation were done at 300×g for 3 minutes at room temperature. Following a first wash in 200 μl PBS, cells were resuspended in 100 μL viability stain (1:4000 Cisplatin-198Pt in PBS) and incubated for 5 minutes at room temperature. Cells were washed twice in 200 μl Cell Staining Buffer.

Cells were resuspended in 45 μL Cell Staining Buffer with 5 μL Human TruStain FcX and incubated for 10 minutes at room temperature. 50 μL of primary surface antibody cocktail was then directly added to the cell suspension at antibody dilutions empirically determined during panel optimization and incubated for 30 minutes at room temperature. During this step, each sample was stained with CD45 antibodies labeled with Cadmium isotope for live barcoding and subsequent multiplexing for CyTOF acquisition. Cells were then washed twice in 200 μl Cell Staining Buffer, resuspended in 100 μL Cell Staining Buffer containing 2 μL anti-FITC and incubated for 20 minutes at room temperature.

Cells were then washed twice in 200 μl Cell Staining Buffer, resuspended in 100 uL freshly prepared 1× Fix/Perm solution and incubated at 4° C. for 45 minutes. All centrifugation steps after fixation were done at 800×g for 3 minutes at room temperature. Following incubation, cells were washed twice with 100 μL 1× Perm/Wash buffer and 50 μL of intracellular antibody cocktail was added to the cell suspension at antibody dilutions empirically determined during panel optimization and incubated for 30 minutes at room temperature. Cells were washed twice with 200 μL 1× Perm/Wash buffer and resuspended in 200 μL DNA Intercalator solution at 1:2000 in MaxPar Fix/Perm Buffer and incubated at 4° C. overnight.

CyTOF2 Acquisition

On the day of mass cytometry acquisition, cells were washed twice in Cell Staining Buffer, followed by two washes in milliQ water. All wash volumes were 200 μL and centrifugation was at 800×g for 3 min at room temperature. Samples were acquired on the CyTOF 2.0 (Fluidigm) after instrument setup and calibration procedures. A custom mass cytometry panel was used for analysis (Table 12) with 42 channels detecting unique proteins, 2 additional channels for CD45 barcoding, and 2 cell ID markers.

TABLE 12
Custom Mass Cytometry Panel

| Live WBC | | B | | NK | T | Differentiation | | Effector | CD226 | Other |
|---|---|---|---|---|---|---|---|---|---|---|
| 10 | PMN | cells | Myeloid | cells | cells | & Memory | | status | axis | (IO) |
| DNA (anterc.) | CD15 | CD19 | CD11b | NKG2D | CD3 | CD45RD | CD57 | Granz. A | CD96 | PD-1 |
| Cisplatin | | | CD11c | CD56 | CD4 | CD45RA | CD101 | Granz. B | CD226 | PD-L1 |
| CD45 | | | CD14 | CD16 | CD8 | TCF1 | KLRG1 | CD25 | TIGIT | CCR8 |
| | | | CD208 | | FoxP3 | Tbet | CX3CR1 | CD38 | CD155 | ICOS |
| | | | HLA-DR | | | CD27 | | IFNγ | CD112 | |
| | | | | | | CD26 | | KI67 | PVRIG\hat{ } | |
| | | | | | | CD127 | | CD107a | | |

\hat{ } PVRIG was stained after cell permeabilization and was only integrated into the panel after the first 43 dissociated tumor cells (DTC) were studied.

Data Acquisition and Analysis Workflow

Samples were acquired on a CyTOF2 and Super Sampler. Flow cytometry standard (FCS) pre-processing was conducted using Fluidigm CyTOF Software (randomization, normalization, and concatenation). Downstream analysis and visualization were performed using FlowJo (for uniform manifold approximation and projection (UMAP) and biaxial gating) and Array Studio and Morpheus (for co-expression and correlations).

Results and Conclusions

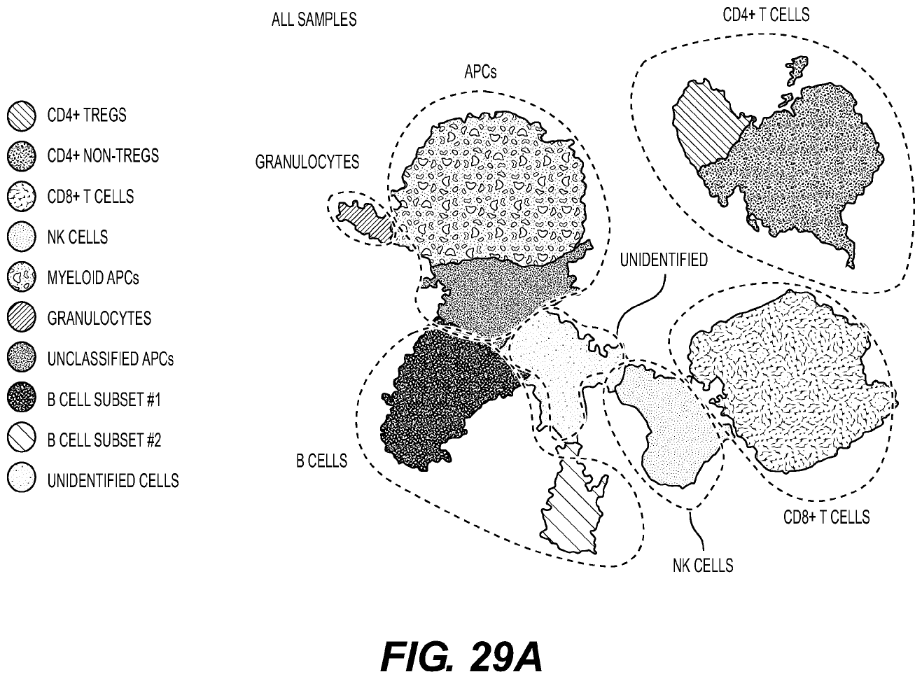

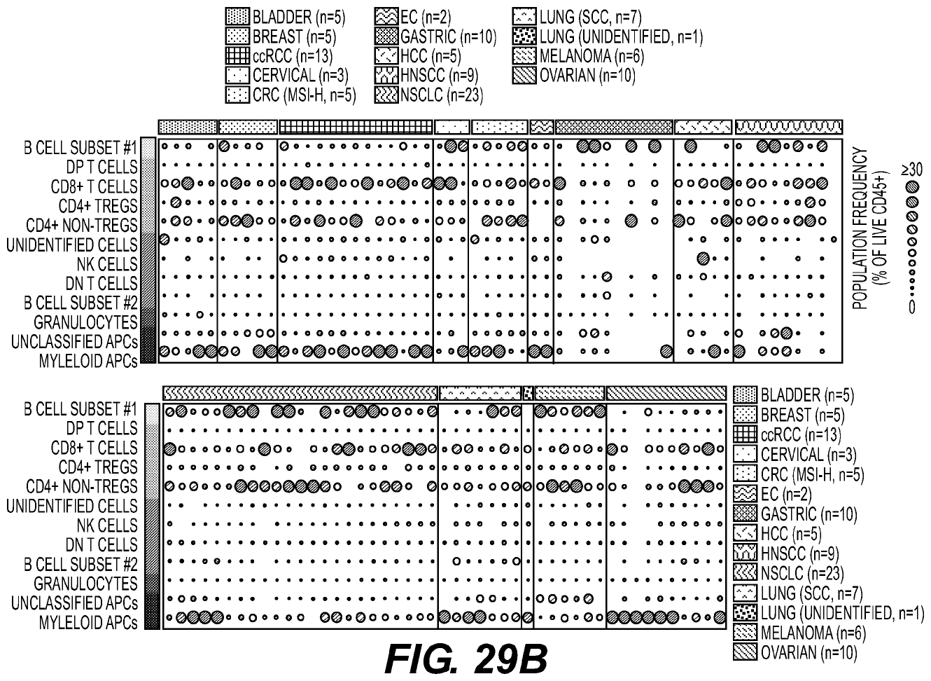

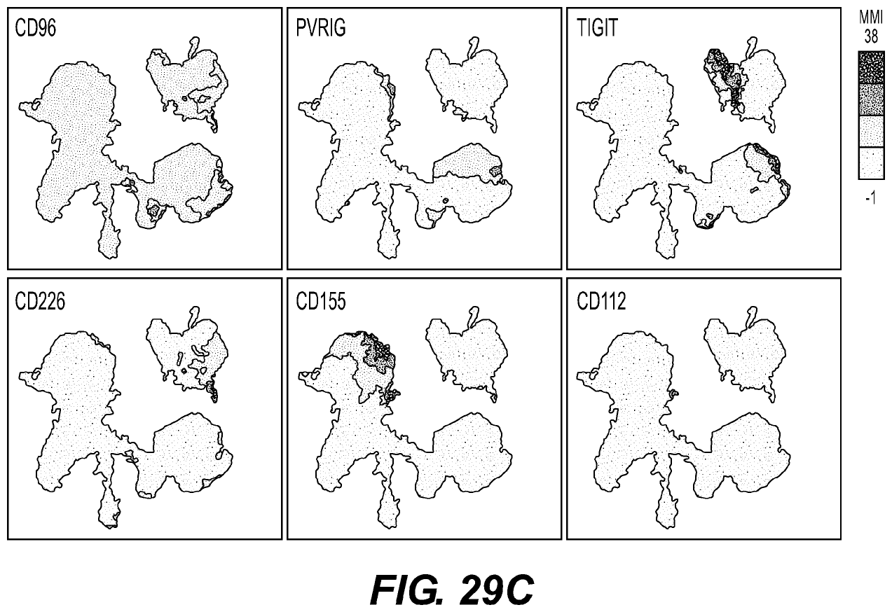

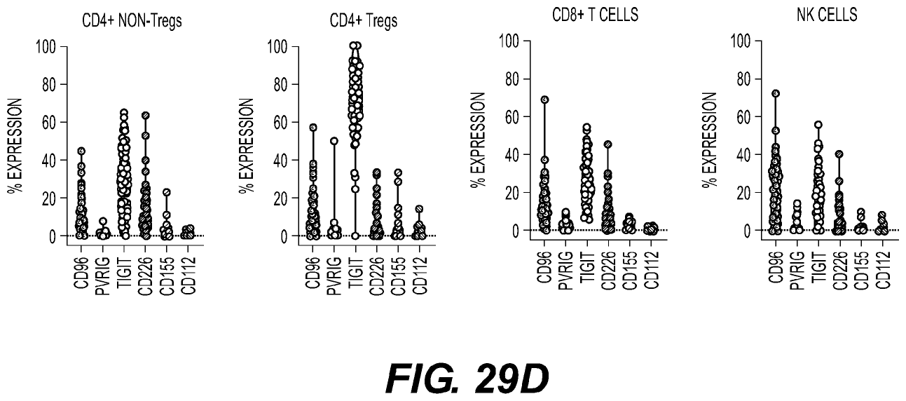

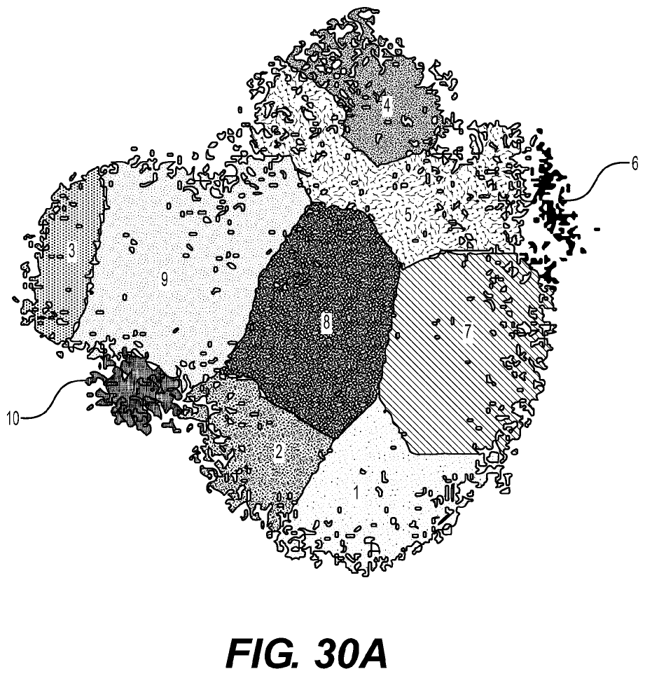

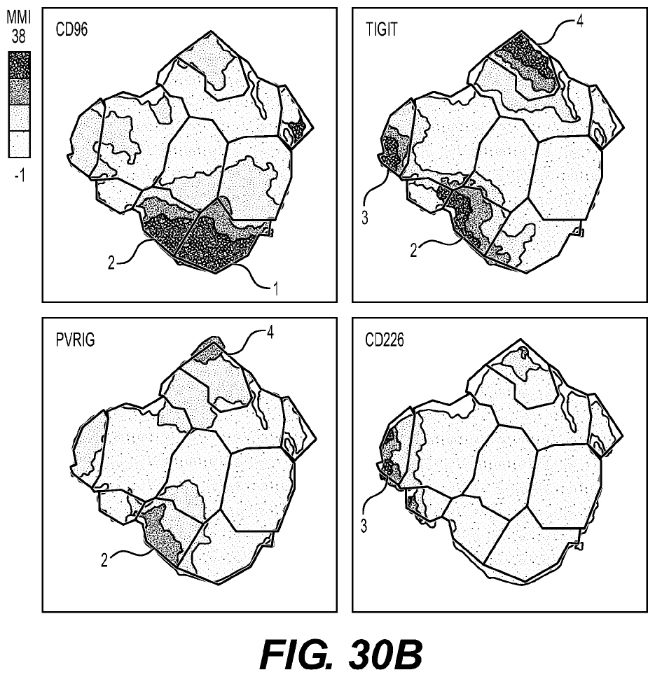

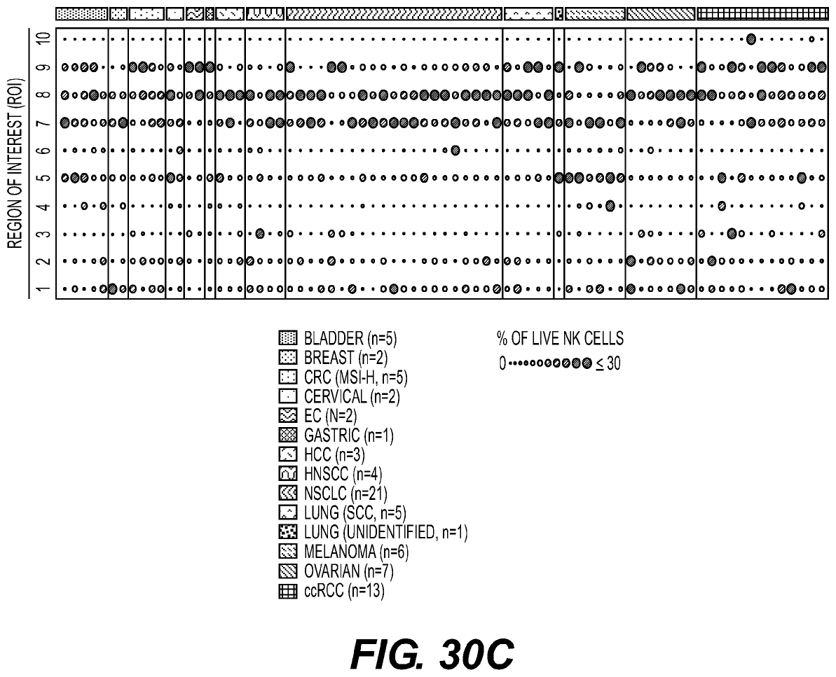

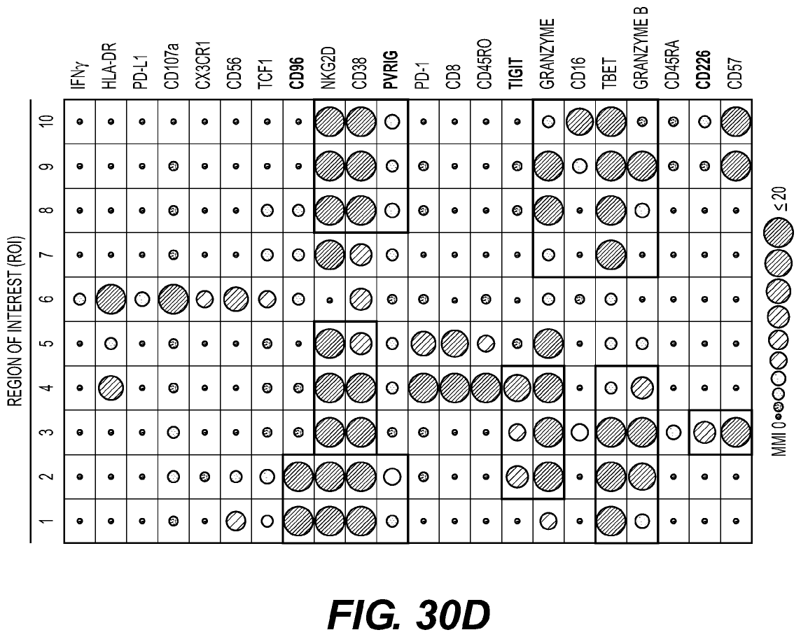

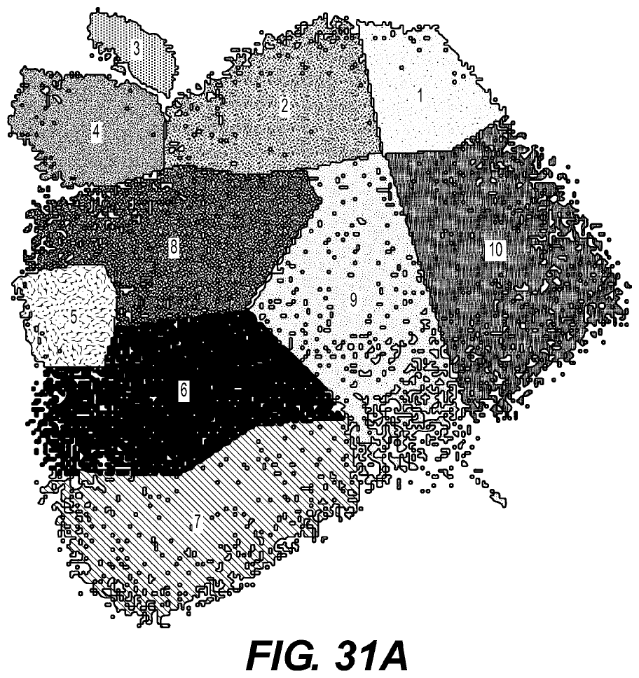

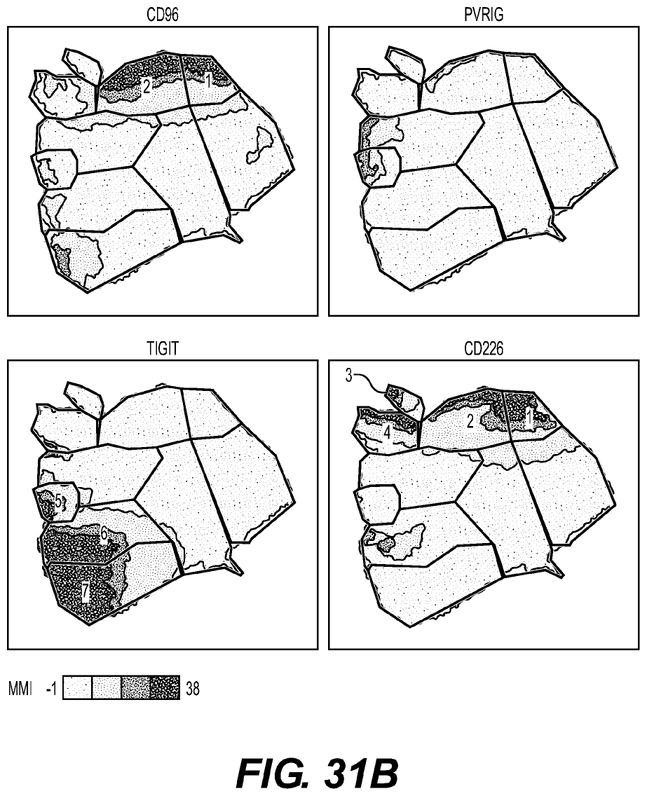

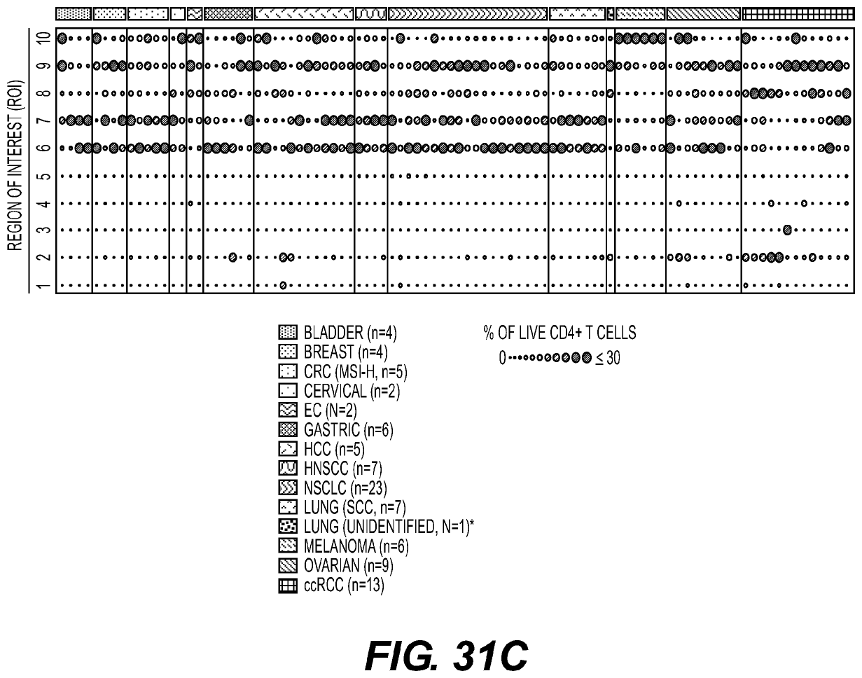

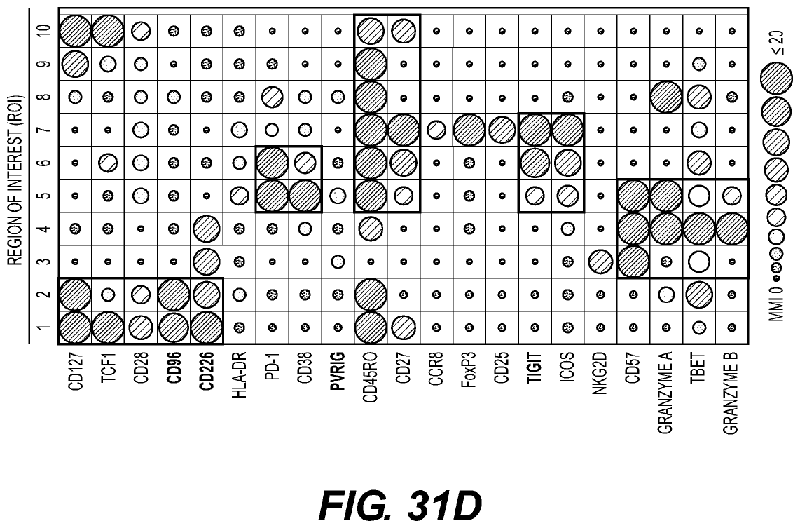

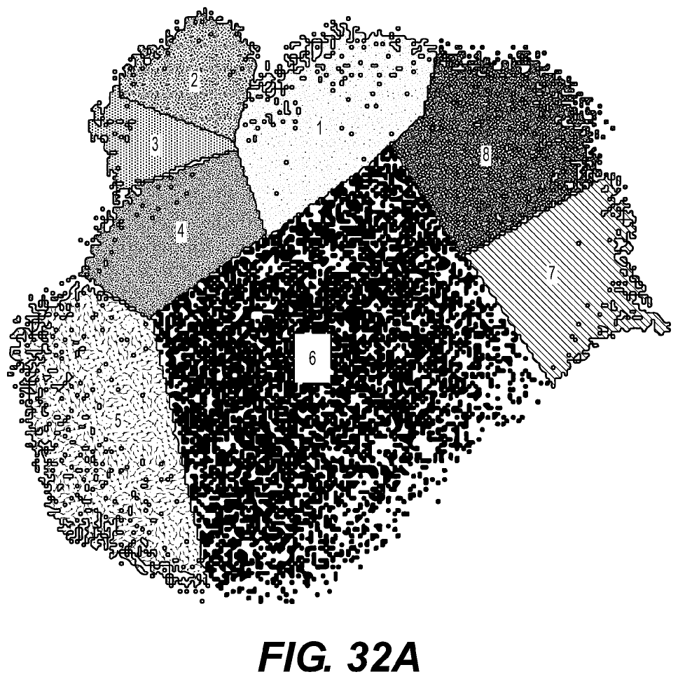

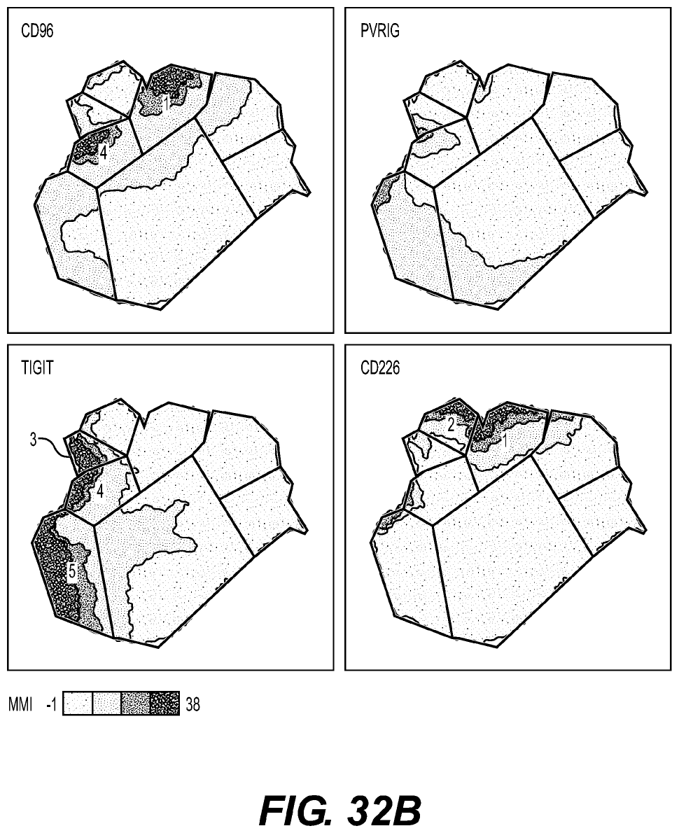

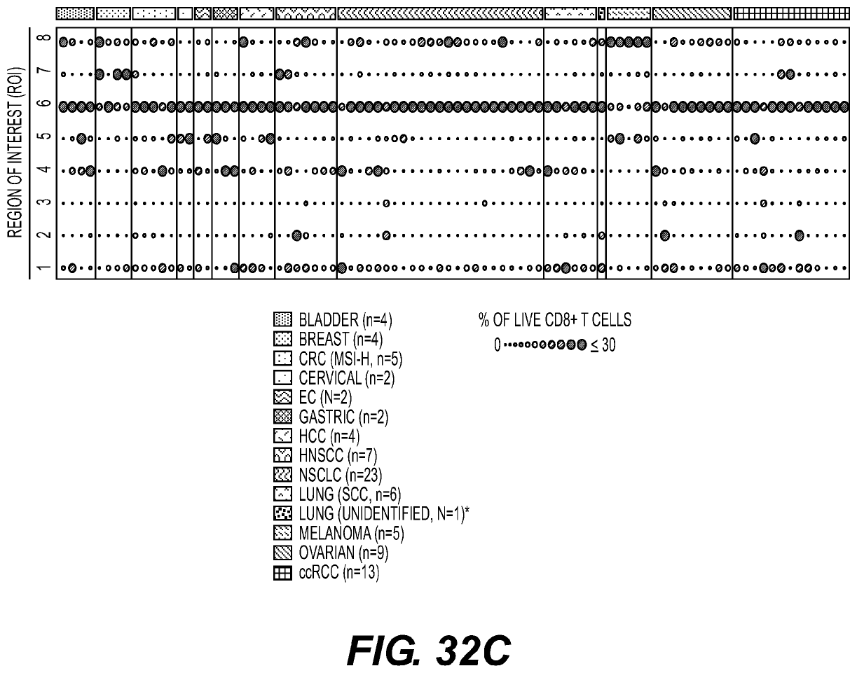

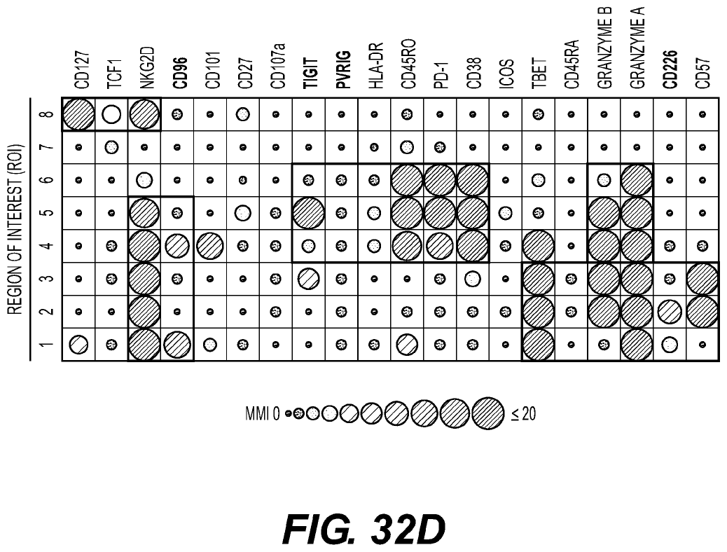

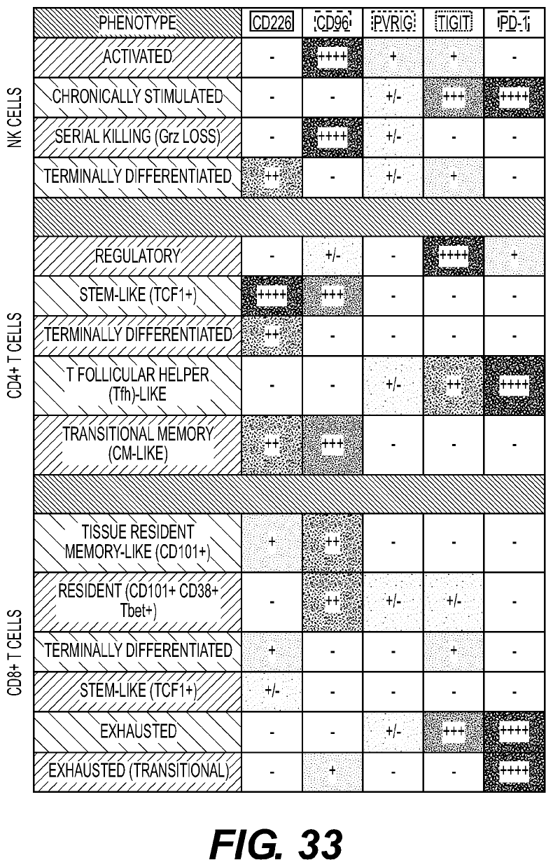

The results of the expression profiling analyses described above are shown in FIGS. 29-33.

Observations regarding immune populations (more broadly): Select clusters of T/NK cells expressing CD226 axis members were found across patient samples, and while select populations were found to be uniquely associated with certain indications (FIGS. 29A-29D), most indications exhibited notable heterogeneity.

CD226 axis observations: TIGIT was most notably associated with highly activated T cells/Tregs. TIGIT correlated more closely with PD-1 relative to CD96/PVRIG/CD226. TIGIT was found on chronically stimulated NK cells alongside PD-1.

CD96 was found on stem-like (TCF 1+) memory T cells (both CD4+ and CD8+) and various populations of activated/serial killer NK cells. CD96 was most highly expressed on NK cells, but was also found on various T cell subsets.

CD226 expression was relatively distinct amongst axis members, but did correlate with CD96 on certain T cell populations (particularly CD4+).

PVRIG was expressed at low levels across NK and CD8+ T cell populations. Additional resolution is needed to find unique PVRIG-expressing immune populations.

CD155 exhibited broad myeloid/APC expression, whereas appreciable CD112 expression was not detected on immune populations.

SEQUENCE LISTINGS

Kabat defined CDRs are underlined.
42Y073-86F08-1 variable light chain
amino acid sequence
SEQ ID NO: 1
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-1 variable heavy chain
amino acid sequence
SEQ ID NO: 2
QVQLVQSGAEVKKPGSSVKVSCKASGGTFSSYAISWVRQAPGQGL
EWMGGIIPIFGTASYAQKFQGRVTITADESTSTAYMELSSLRSED
TAVYYCARGAGYYGDKDPMDVWGQGTTVTVSS
42Y073-86F08-1 variable light chain
nucleic acid sequence
SEQ ID NO: 3
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-1 variable heavy chain
nucleic acid sequence
SEQ ID NO: 4
CAGGTGCAGCTGGTGCAGAGCGGCGCCGAGGTGAAAAAGCCCGGC
AGCAGCGTGAAGGTGAGCTGCAAGGCCTCCGGGGGGACCTTCAGC
AGCTACGCCATCAGCTGGGTGAGGCAGGCTCCCGGACAGGGCCTG
GAGTGGATGGGGGGCATCATCCCCATTTTCGGCACCGCCAGCTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGCGCCGGCTACTACGGCGAC
AAGGACCCCATGGACGTGTGGGGCCAGGGCACCACCGTGACTGTG
AGCAGC
42Y073-86F04-3 variable light chain
amino acid sequence
SEQ ID NO: 5
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIK
42Y073-86F04-3 variable heavy chain
amino acid sequence
SEQ ID NO: 6
QVQLVQSGAEVKKPGSSVKVSCKASGGTFSYNAISWVRQAPGQGL
EWMGGIIPIMGTARYAQKFQGRVTITADESTSTAYMELSSLRSED
TAVYYCARLLGESGMDVWGQGTTVTVSS
42Y073-86F04-3 variable light chain
nucleic acid sequence
SEQ ID NO: 7
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
CCCTACTTCAGCCCCCCCACTTTCGGCGGCGGCACCAAGGTGGAG
ATTAAG
42Y073-86F04-3 variable heavy chain
nucleic acid sequence
SEQ ID NO: 8
CAGGTGCAGCTGGTGCAGAGCGGCGCCGAGGTGAAAAAGCCCGGC
AGCAGCGTGAAGGTGAGCTGCAAGGCCTCCGGCGGGACCTTCAGC
TACAACGCCATCAGCTGGGTGAGGCAGGCTCCCGGACAGGGCCTG
GAGTGGATGGGCGGCATCATCCCCATTATGGGCACCGCCAGGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGCTGCTGGGCGAGAGCGGCATG
GACGTGTGGGGCCAGGGCACCACCGTGACTGTGAGCAGC
42Y073-86F04-4 variable light chain
amino acid sequence
SEQ ID NO: 9
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIK
42Y073-86F04-4 variable heavy chain
amino acid sequence
SEQ ID NO: 10
QVQLVQSGAEVKKPGSSVKVSCKASGGTFESEAISWVRQAPGQGL
EWMGGIIPIFGRARYAQKFQGRVTITADESTSTAYMELSSLRSED
TAVYYCARLLGESGMDVWGQGTTVTVSS
42Y073-86F04-4 variable light chain
nucleic acid sequence
SEQ ID NO: 11
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
CCCTACTTCAGCCCCCCCACTTTCGGCGGCGGCACCAAGGTGGAG
ATTAAG
42Y073-86F04-4 variable heavy chain
nucleic acid sequence
SEQ ID NO: 12
CAGGTGCAGCTGGTGCAGAGCGGCGCCGAGGTGAAAAAGCCCGGC
AGCAGCGTGAAGGTGAGCTGCAAGGCCTCCGGCGGGACCTTCGAG
AGCGAGGCCATCAGCTGGGTGAGGCAGGCTCCCGGACAGGGCCTG
GAGTGGATGGGCGGCATCATCCCCATTTTCGGCAGGGCCAGGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGCTGCTGGGCGAGAGCGGCATG
GACGTGTGGGGCCAGGGCACCACCGTGACTGTGAGCAGC
42Y073-86F04-5 variable light chain
amino acid sequence
SEQ ID NO: 13
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIK
42Y073-86F04-5 variable heavy chain
amino acid sequence
SEQ ID NO: 14
QVQLVQSGAEVKKPGSSVKVSCKASGGTFSSHAISWVRQAPGQGL
EWMGGIIPIFGRGKYAQKFQGRVTITADESTSTAYMELSSLRSED
TAVYYCARLLGESGMDVWGQGTTVTVSS
42Y073-86F04-5 variable light chain
nucleic acid sequence
SEQ ID NO: 15
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
CCCTACTTCAGCCCCCCCACTTTCGGCGGCGGCACCAAGGTGGAG
ATTAAG
42Y073-86F04-5 variable heavy chain
nucleic acid sequence
SEQ ID NO: 16
CAGGTGCAGCTGGTGCAGAGCGGCGCCGAGGTGAAAAAGCCCGGC
AGCAGCGTGAAGGTGAGCTGCAAGGCCTCCGGGGGGACCTTCAGC
AGCCACGCCATCAGCTGGGTGAGGCAGGCTCCCGGACAGGGCCTG
GAGTGGATGGGCGGCATCATCCCCATTTTCGGCAGGGGCAAGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGCTGCTGGGCGAGAGCGGCATG
GACGTGTGGGGCCAGGGCACCACCGTGACTGTGAGCAGC
42Y073-86F04-6 variable light chain
amino acid sequence
SEQ ID NO: 17
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIK
42Y073-86F04-6 variable heavy chain
amino acid sequence
SEQ ID NO: 18
QVQLVQSGAEVKKPGSSVKVSCKASGGTFSGHAISWVRQAPGQGL
EWMGGIIPIFGRARYAQKFQGRVTITADESTSTAYMELSSLRSED
TAVYYCARLLGESGMDVWGQGTTVTVSS
42Y073-86F04-6 variable light chain
nucleic acid sequence
SEQ ID NO: 19
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
CCCTACTTCAGCCCCCCCACTTTCGGCGGCGGCACCAAGGTGGAG
ATTAAG
42Y073-86F04-6 variable heavy chain
nucleic acid sequence
SEQ ID NO: 20
CAGGTGCAGCTGGTGCAGAGCGGCGCCGAGGTGAAAAAGCCCGGC
AGCAGCGTGAAGGTGAGCTGCAAGGCCTCCGGCGGGACCTTCAGC
GGCCACGCCATCAGCTGGGTGAGGCAGGCTCCCGGACAGGGCCTG
GAGTGGATGGGCGGCATCATCCCCATTTTCGGCAGGGCCAGGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGCTGCTGGGCGAGAGCGGCATG
GACGTGTGGGGCCAGGGCACCACCGTGACTGTGAGCAGC
42Y073-86F04-18 variable light chain
amino acid sequence
SEQ ID NO: 21
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIK
42Y073-86F04-18 variable heavy chain
amino acid sequence
SEQ ID NO: 22
QVQLVQSGAEVKKPGSSVKVSCKASGGTFSSRAISWVRQAPGQGL
EWMGGIIPIMGTARYAQKFQGRVTITADESTSTAYMELSSLRSED
TAVYYCARLLGESGMDVWGQGTTVTVSS
42Y073-86F04-18 variable light chain
nucleic acid sequence
SEQ ID NO: 23
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
CCCTACTTCAGCCCCCCCACTTTCGGCGGCGGCACCAAGGTGGAG
ATTAAG
42Y073-86F04-18 variable heavy chain
nucleic acid sequence
SEQ ID NO: 24
CAGGTGCAGCTGGTGCAGAGCGGCGCCGAGGTGAAAAAGCCCGGC
AGCAGCGTGAAGGTGAGCTGCAAGGCCTCCGGCGGGACCTTCAGC
AGCAGGGCCATCAGCTGGGTGAGGCAGGCTCCCGGACAGGGCCTG
GAGTGGATGGGGGCATCATCCCCATTATGGGCACCGCCAGGTACG
CCCAGAAGTTCCAGGGAAGGGTCACCATCACCGCCGACGAGAGCA
CCAGCACCGCCTACATGGAACTCAGCAGCCTGAGGAGCGAGGACA
CCGCCGTGTACTATTGCGCCAGGCTGCTGGGCGAGAGCGGCATGG
ACGTGTGGGGCCAGGGCACCACCGTGACTGTGAGCAGC
42Y073-86F04-33 variable light chain
amino acid sequence
SEQ ID NO: 25
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIK
42Y073-86F04-33 variable heavy chain
amino acid sequence
SEQ ID NO: 26
QVQLVQSGAEVKKPGSSVKVSCKASGGTFSSYAISWVRQAPGQGL
EWMGGILPIFGRANYAQKFQGRVTITADESTSTAYMELSSLRSED
TAVYYCARLLGESGMDVWGQGTTVTVSS
42Y073-86F04-33 variable light chain
nucleic acid sequence
SEQ ID NO: 27
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
CCCTACTTCAGCCCCCCCACTTTCGGCGGCGGCACCAAGGTGGAG
ATTAAG
42Y073-86F04-33 variable heavy chain
nucleic acid sequence
SEQ ID NO: 28
CAGGTGCAGCTGGTGCAGAGCGGCGCCGAGGTGAAAAAGCCCGGC
AGCAGCGTGAAGGTGAGCTGCAAGGCCTCCGGGGGGACCTTCAGC
AGCTACGCCATCAGCTGGGTGAGGCAGGCTCCCGGACAGGGCCTG
GAGTGGATGGGCGGCATCCTGCCCATTTTCGGCAGGGCCAACTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGCTGCTGGGCGAGAGCGGCATG
GACGTGTGGGGCCAGGGCACCACCGTGACTGTGAGCAGC
42Y073-86F04-88 variable light chain
amino acid sequence
SEQ ID NO: 29
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIK
42Y073-86F04-88 variable heavy chain
amino acid sequence
SEQ ID NO: 30
QVQLVQSGAEVKKPGSSVKVSCKASGGTFSSSAISWVRQAPGQGL
EWMGGIIPIFGRANYAQKFQGRVTITADESTSTAYMELSSLRSED
TAVYYCARLLGESGMDVWGQGTTVTVSS
42Y073-86F04-88 variable light chain
nucleic acid sequence
SEQ ID NO: 31
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
CCCTACTTCAGCCCCCCCACTTTCGGCGGCGGCACCAAGGTGGAG
ATTAAG
42Y073-86F04-88 variable heavy chain
nucleic acid sequence
SEQ ID NO: 32
CAGGTGCAGCTGGTGCAGAGCGGCGCCGAGGTGAAAAAGCCCGGC
AGCAGCGTGAAGGTGAGCTGCAAGGCCTCCGGCGGGACCTTCAGC
AGCAGCGCCATCAGCTGGGTGAGGCAGGCTCCCGGACAGGGCCTG
GAGTGGATGGGCGGCATCATCCCCATTTTCGGCAGGGCCAACTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGCTGCTGGGCGAGAGCGGCATG
GACGTGTGGGGCCAGGGCACCACCGTGACTGTGAGCAGC
42Y073-86F08-3 variable light chain
amino acid sequence
SEQ ID NO: 33
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-3 variable heavy chain
amino acid sequence
SEQ ID NO: 34
QVQLVQSGAEVKKPGSSVKVSCKASGGTFVNYAISWVRQAPGQGL
EWMGGIIPALGTANYAQKFQGRVTITADESTSTAYMELSSLRSED
TAVYYCARGAGYYGDKDPMDVWGQGTTVTVSS
42Y073-86F08-3 variable light chain
nucleic acid sequence
SEQ ID NO: 35
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-3 variable heavy chain
nucleic acid sequence
SEQ ID NO: 36
CAGGTGCAGCTGGTGCAGAGCGGCGCCGAGGTGAAAAAGCCCGGC
AGCAGCGTGAAGGTGAGCTGCAAGGCCTCCGGGGGGACCTTCGTG
AACTACGCCATCAGCTGGGTGAGGCAGGCTCCCGGACAGGGCCTG
GAGTGGATGGGCGGCATCATCCCCGCCCTGGGCACCGCCAACTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGCGCCGGCTACTACGGCGAC
AAGGACCCCATGGACGTGTGGGGCCAGGGCACCACCGTGACTGTG
AGCAGC
42Y073-86F08-4 variable light chain
amino acid sequence
SEQ ID NO: 37
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-4 variable heavy chain
amino acid sequence
SEQ ID NO: 38
QVQLVQSGAEVKKPGSSVKVSCKASGGTFSEYAIHWVRQAPGQGL
EWMGNIIPIFGTAGYAQKFQGRVTITADESTSTAYMELSSLRSED
TAVYYCARGAGYYGDKDPMDVWGQGTTVTVSS
42Y073-86F08-4 variable light chain
nucleic acid sequence
SEQ ID NO: 39
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-4 variable heavy chain
nucleic acid sequence
SEQ ID NO: 40
CAGGTGCAGCTGGTGCAGAGCGGCGCCGAGGTGAAAAAGCCCGGC
AGCAGCGTGAAGGTGAGCTGCAAGGCCTCCGGGGGGACCTTCAGC
GAGTACGCCATCCACTGGGTGAGGCAGGCTCCCGGACAGGGCCTG
GAGTGGATGGGCAACATCATCCCCATTTTCGGCACCGCCGGCTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGCGCCGGCTACTACGGCGAC
AAGGACCCCATGGACGTGTGGGGCCAGGGCACCACCGTGACTGTG
AGCAGC
42Y073-86F08-8 variable light chain
amino acid sequence
SEQ ID NO: 41
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-8 variable heavy chain
amino acid sequence
SEQ ID NO: 42
QVQLVQSGAEVKKPGSSVKVSCKASGGTFWLYAISWVRQAPGQGL
EWMGGIIPQLGTANYAQKFQGRVTITADESTSTAYMELSSLRSED
TAVYYCARGAGYYGDKDPMDVWGQGTTVTVSS
42Y073-86F08-8 variable light chain
nucleic acid sequence
SEQ ID NO: 43
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-8 variable heavy chain
nucleic acid sequence
SEQ ID NO: 44
CAGGTGCAGCTGGTGCAGAGCGGCGCCGAGGTGAAAAAGCCCGGC
AGCAGCGTGAAGGTGAGCTGCAAGGCCTCCGGGGGGACCTTCTGG
CTGTACGCCATCAGCTGGGTGAGGCAGGCTCCCGGACAGGGCCTG
GAGTGGATGGGCGGCATCATCCCCCAGCTGGGCACCGCCAACTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGCGCCGGCTACTACGGCGAC
AAGGACCCCATGGACGTGTGGGGCCAGGGCACCACCGTGACTGTG
AGCAGC
42Y073-86F08-17 variable light chain
amino acid sequence
SEQ ID NO: 45
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-17 variable heavy chain
amino acid sequence
SEQ ID NO: 46
QVQLVQSGAEVKKPGSSVKVSCKASGGTFREYAISWVRQAPGQGL
EWMGGIIPVFGTANYAQKFQGRVTITADESTSTAYMELSSLRSED
TAVYYCARGAGYYGDKDPMDVWGQGTTVTVSS
42Y073-86F08-17 variable light chain
nucleic acid sequence
SEQ ID NO: 47
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-17 variable heavy chain
nucleic acid sequence
SEQ ID NO: 48
CAGGTGCAGCTGGTGCAGAGCGGCGCCGAGGTGAAAAAGCCCGGC
AGCAGCGTGAAGGTGAGCTGCAAGGCCTCCGGGGGGACCTTCAGG
GAGTACGCCATCAGCTGGGTGAGGCAGGCTCCCGGACAGGGCCTG
GAGTGGATGGGCGGCATCATCCCCGTGTTCGGCACCGCCAACTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGCGCCGGCTACTACGGCGAC
AAGGACCCCATGGACGTGTGGGGCCAGGGCACCACCGTGACTGTG
AGCAGC
42Y073-86F08-22 variable light chain
amino acid sequence
SEQ ID NO: 49
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-22 variable heavy chain
amino acid sequence
SEQ ID NO: 50
QVQLVQSGAEVKKPGASVKVSCKASGYTFDSYAMHWVRQAPGQGL
EWMGGIIPIFGTAWYAQKFQGRVTITADESTSTAYMELSSLRSED
TAVYYCARGAGYYGDKDPMDVWGQGTTVTVSS
42Y073-86F08-22 variable light chain
nucleic acid sequence
SEQ ID NO: 51
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-22 variable heavy chain
nucleic acid sequence
SEQ ID NO: 52
CAGGTGCAGCTGGTGCAGAGCGGCGCCGAGGTGAAAAAGCCCGGC
GCCAGCGTGAAGGTGAGCTGCAAGGCCTCCGGCTACACCTTCGAC
AGCTACGCCATGCACTGGGTGAGGCAGGCTCCCGGACAGGGCCTG
GAGTGGATGGGCGGCATCATCCCCATTTTCGGCACCGCCTGGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGCGCCGGCTACTACGGCGAC
AAGGACCCCATGGACGTGTGGGGCCAGGGCACCACCGTGACTGTG
AGCAGC
42Y073-86F08-47 variable light chain
amino acid sequence
SEQ ID NO: 53
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-47 variable heavy chain
amino acid sequence
SEQ ID NO: 54
QVQLVQSGAEVKKPGSSVKVSCKASGGTFSQYAIHYWVRQAPGQG
LEWMGVIIPIFGKANYAQKFQGRVTITADESTSTAYMELSSLRSE
DTAVYYCARGAGYYGDKDPMDVWGQGTTVTVSS
42Y073-86F08-47 variable light chain
nucleic acid sequence
SEQ ID NO: 55
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-47 variable heavy chain
nucleic acid sequence
SEQ ID NO: 56
CAGGTGCAGCTGGTGCAGAGCGGCGCCGAGGTGAAAAAGCCCGGC
AGCAGCGTGAAGGTGAGCTGCAAGGCCTCCGGGGGGACCTTCAGC
CAGTACGCCATCCACTGGGTGAGGCAGGCTCCCGGACAGGGCCTG
GAGTGGATGGGCGTGATCATCCCCATTTTCGGCAAGGCCAACTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGCGCCGGCTACTACGGCGAC
AAGGACCCCATGGACGTGTGGGGCCAGGGCACCACCGTGACTGTG
AGCAGC
42Y073-2B04-46 variable light chain
amino acid sequence
SEQ ID NO: 57
EIVLTQSPATLSLSPGERATLSCRASQSVSSYLAWYQQKPGQAPR
LLIYDASKRATGIPARFSGSGSGTDFTLTISSLEPEDFAVYYCQQ
LDNWPITFGGGTKVEIK
42Y073-2B04-46 variable heavy chain
amino acid sequence
SEQ ID NO: 58
QVQLQQWGAGLLKPSETLSLTCAVYGGSFRRYYWSWIRQPPGKGL
EWIGEIDGWGSTNYNPSLKSRVTISVDTSKNQFSLKLSSVTAADT
AVYYCARGGSVDFWSGSDYYYYMDVWGKGATVTVSS
42Y073-2B04-46 variable light chain
nucleic acid sequence
SEQ ID NO: 59
GAGATCGTGCTGACCCAGAGCCCCGCAACCCTGTCCCTGAGCCCC
GGCGAAAGGGCCACTCTGAGCTGCAGGGCCAGCCAGAGCGTGAGC
AGCTACCTCGCCTGGTACCAGCAGAAGCCCGGCCAGGCCCCTAGG
CTGCTGATCTACGACGCCAGCAAGAGGGCCACCGGCATTCCCGCC
AGGTTCAGCGGCAGCGGCAGCGGCACCGACTTCACCCTGACCATC
AGCAGCCTGGAGCCCGAGGACTTCGCCGTCTACTACTGCCAGCAG
CTGGACAACTGGCCCATCACCTTCGGGGGGGGCACCAAGGTGGAG
ATCAAG
42Y073-2B04-46 variable heavy chain
nucleic acid sequence
SEQ ID NO: 60
CAGGTGCAGCTGCAGCAGTGGGGCGCCGGACTGCTGAAGCCCAGC
GAGACCCTGAGCCTGACCTGCGCCGTGTACGGCGGGTCCTTCAGG
AGGTACTACTGGAGCTGGATCAGGCAGCCCCCCGGCAAAGGCCTG
GAGTGGATCGGCGAGATCGACGGCTGGGGCAGCACCAACTACAAC
CCCAGCCTCAAGAGCAGGGTGACCATCAGCGTGGACACCAGCAAG
AACCAGTTCAGCCTGAAGCTGAGCAGCGTGACCGCCGCCGACACC
GCCGTGTACTATTGCGCCAGGGGCGGCAGCGTGGACTTCTGGAGC
GGCAGCGACTACTACTACTACATGGACGTGTGGGGCAAGGGCGCC
ACCGTCACCGTGAGCAGC
42Y073-1A01-85 variable light chain
amino acid sequence
SEQ ID NO: 61
DIQMTQSPSSLSASVGDRVTITCRASQSISSYLNWYQQKPGKAPK
LLIYAASSLQSGVPSRFSGSGSGTDFTLTISSLQPEDFATYYCQQ
SLYPPRTFGGGTKVEIK
42Y073-1A01-85 variable heavy chain
amino acid sequence
SEQ ID NO: 62
QVQLVQSGAEVKKPGASVKVSCKASGYTFTSYPMHWVRQAPGQGL
EWMGIINPSGGFTSYAQKFQGRVTMTRDTSTSTVYMELSSLRSED
TAVYYCARETAYYTTKGNWFDPWGQGTLVTVSA
42Y073-1A01-85 variable light chain
nucleic acid sequence
SEQ ID NO: 63
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
AGCCTGTACCCCCCCAGGACTTTCGGCGGCGGCACCAAGGTGGAG
ATTAAG
42Y073-1A01-85 variable heavy chain
nucleic acid sequence
SEQ ID NO: 64
CAGGTGCAGCTGGTGCAGAGCGGCGCCGAAGTGAAAAAGCCCGGC
GCCAGCGTGAAGGTCAGCTGCAAGGCCTCCGGGTACACCTTCACC
AGCTACCCCATGCACTGGGTGAGGCAGGCCCCCGGCCAGGGCCTC
GAGTGGATGGGCATCATCAACCCCAGCGGAGGCTTCACCAGCTAC
GCCCAGAAGTTCCAGGGCAGGGTGACCATGACAAGGGACACCAGC
ACCAGCACCGTGTACATGGAGCTGAGCAGCCTGAGGAGCGAGGAC
ACCGCCGTGTATTACTGCGCAAGGGAGACCGCCTACTACACCACC
AAGGGCAACTGGTTCGACCCCTGGGGCCAGGGCACCCTGGTGACC
GTGAGCGCC
42Y073-1A01-97 variable light chain
amino acid sequence
SEQ ID NO: 65
DIQMTQSPSSLSASVGDRVTITCRASQSISSYLNWYQQKPGKAPK
LLIYAASSLQSGVPSRFSGSGSGTDFTLTISSLQPEDFATYYCQQ
SLYPPRTFGGGTKVEIK
42Y073-1A01-97 variable heavy chain
amino acid sequence
SEQ ID NO: 66
QVQLVQSGAEVKKPGASVKVSCKASGYTFTRAAMHWVRQAPGQGL
EWMGIINPAGGYTSYAQKFQGRVTMTRDTSTSTVYMELSSLRSED
TAVYYCARETAYYTTKGNWFDPWGQGTLVTVSS
42Y073-1A01-97 variable light chain
nucleic acid sequence
SEQ ID NO: 67
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
AGCCTGTACCCCCCCAGGACTTTCGGCGGCGGCACCAAGGTGGAG
ATTAAG
42Y073-1A01-97 variable heavy chain
nucleic acid sequence
SEQ ID NO: 68
CAGGTGCAGCTGGTGCAGAGCGGCGCCGAAGTGAAAAAGCCCGGC
GCCAGCGTGAAGGTCAGCTGCAAGGCCTCCGGGTACACCTTCACC
AGGGCCGCCATGCACTGGGTGAGGCAGGCCCCCGGCCAGGGCCTC
GAGTGGATGGGCATCATCAACCCCGCCGGAGGCTACACCAGCTAC
GCCCAGAAGTTCCAGGGCAGGGTGACCATGACAAGGGACACCAGC
ACCAGCACCGTGTACATGGAGCTGAGCAGCCTGAGGAGCGAGGAC
ACCGCCGTGTATTACTGCGCAAGGGAGACCGCCTACTACACCACC
AAGGGCAACTGGTTCGACCCCTGGGGCCAGGGCACCCTGGTGACC
GTGAGCTCT
42Y073-1A01-100 variable light chain
amino acid sequence
SEQ ID NO: 69
DIQMTQSPSSLSASVGDRVTITCRASQSISSYLNWYQQKPGKAPK
LLIYAASSLQSGVPSRFSGSGSGTDFTLTISSLQPEDFATYYCQQ
SLYPPRTFGGGTKVEIK
42Y073-1A01-100 variable heavy chain
amino acid sequence
SEQ ID NO: 70
QVQLVQSGAEVKKPGASVKVSCKASGYTFTTYRMHWVRQAPGQGL
EWMGIINPSGGLTQYAQKFQGRVTMTRDTSTSTVYMELSSLRSED
TAVYYCARETAYYTTKGNWFDPWGQGTLVTVSS
42Y073-1A01-100 variable light chain
nucleic acid sequence
SEQ ID NO: 71
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
AGCCTGTACCCCCCCAGGACTTTCGGCGGCGGCACCAAGGTGGAG
ATTAAG
42Y073-1A01-100 variable heavy chain
nucleic acid sequence
SEQ ID NO: 72
CAGGTGCAGCTGGTGCAGAGCGGCGCCGAAGTGAAAAAGCCCGGC
GCCAGCGTGAAGGTCAGCTGCAAGGCCTCCGGGTACACCTTCACC
ACCTACAGGATGCACTGGGTGAGGCAGGCCCCCGGCCAGGGCCTC
GAGTGGATGGGCATCATCAACCCCAGCGGAGGCCTGACCCAGTAC
GCCCAGAAGTTCCAGGGCAGGGTGACCATGACAAGGGACACCAGC
ACCAGCACCGTGTACATGGAGCTGAGCAGCCTGAGGAGCGAGGAC
ACCGCCGTGTATTACTGCGCAAGGGAGACCGCCTACTACACCACC
AAGGGCAACTGGTTCGACCCCTGGGGCCAGGGCACCCTGGTGACC
GTGAGCTCT
42Y073-1A01-103 variable light chain
amino acid sequence
SEQ ID NO: 73
DIQMTQSPSSLSASVGDRVTITCRASQSISSYLNWYQQKPGKAPK
LLIYAASSLQSGVPSRFSGSGSGTDFTLTISSLQPEDFATYYCQQ
SLYPPRTFGGGTKVEIK
42Y073-1A01-103 variable heavy chain
amino acid sequence
SEQ ID NO: 74
QVQLVQSGAEVKKPGASVKVSCKASGYTFTAYQMHWVRQAPGQGL
EWMGIINPAGGWTSYAQKFQGRVTMTRDTSTSTVYMELSSLRSED
TAVYYCARETAYYTTKGNWFDPWGQGTLVTVSS
42Y073-1A01-103 variable light chain
nucleic acid sequence
SEQ ID NO: 75
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
AGCCTGTACCCCCCCAGGACTTTCGGCGGCGGCACCAAGGTGGAG
ATTAAG
42Y073-1A01-103 variable heavy chain
nucleic acid sequence
SEQ ID NO: 76
CAGGTGCAGCTGGTGCAGAGCGGCGCCGAAGTGAAAAAGCCCGGC
GCCAGCGTGAAGGTCAGCTGCAAGGCCTCCGGGTACACCTTCACC
GCCTACCAGATGCACTGGGTGAGGCAGGCCCCCGGCCAGGGCCTC
GAGTGGATGGGCATCATCAACCCCGCCGGAGGCTGGACCAGCTAC
GCCCAGAAGTTCCAGGGCAGGGTGACCATGACAAGGGACACCAGC
ACCAGCACCGTGTACATGGAGCTGAGCAGCCTGAGGAGCGAGGAC
ACCGCCGTGTATTACTGCGCAAGGGAGACCGCCTACTACACCACC
AAGGGCAACTGGTTCGACCCCTGGGGCCAGGGCACCCTGGTGACC
GTGAGCTCT
42Y073-1A01-126 variable light chain
amino acid sequence
SEQ ID NO: 77
DIQMTQSPSSLSASVGDRVTITCRASQSISSYLNWYQQKPGKAPK
LLIYAASSLQSGVPSRFSGSGSGTDFTLTISSLQPEDFATYYCQQ
SLYPPRTFGGGTKVEIK
42Y073-1A01-126 variable heavy chain
amino acid sequence
SEQ ID NO: 78
QVQLVQSGAEVKKPGASVKVSCKASGYTFTRYNMHWVRQAPGQGL
EWMGWINPAGGSTSYAQKFQGRVTMTRDTSTSTVYMELSSLRSED
TAVYYCARETAYYTTKGNWFDPWGQGTLVTVSA
42Y073-1A01-126 variable light chain
nucleic acid sequence
SEQ ID NO: 79
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
AGCCTGTACCCCCCCAGGACTTTCGGCGGCGGCACCAAGGTGGAG
ATTAAG
42Y073-1A01-126 variable heavy chain
nucleic acid sequence
SEQ ID NO: 80
CAGGTGCAGCTGGTGCAGAGCGGCGCCGAAGTGAAAAAGCCCGGC
GCCAGCGTGAAGGTCAGCTGCAAGGCCTCCGGGTACACCTTCACC
AGGTACAACATGCACTGGGTGAGGCAGGCCCCCGGCCAGGGCCTC
GAGTGGATGGGCTGGATCAACCCCGCCGGAGGCAGCACCAGCTAC
GCCCAGAAGTTCCAGGGCAGGGTGACCATGACAAGGGACACCAGC
ACCAGCACCGTGTACATGGAGCTGAGCAGCCTGAGGAGCGAGGAC
ACCGCCGTGTATTACTGCGCAAGGGAGACCGCCTACTACACCACC
AAGGGCAACTGGTTCGACCCCTGGGGCCAGGGCACCCTGGTGACC
GTGAGCGCC
42Y073-1A01-191 variable light chain
amino acid sequence
SEQ ID NO: 81
DIQMTQSPSSLSASVGDRVTITCRASQSISSYLNWYQQKPGKAPK
LLIYAASSLQSGVPSRFSGSGSGTDFTLTISSLQPEDFATYYCQQ
SLYPPRTFGGGTKVEIK
42Y073-1A01-191 variable heavy chain
amino acid sequence
SEQ ID NO: 82
QVQLVQSGAEVKKPGASVKVSCKASGYTFTKYRMHWVRQAPGQGL
EWMGIINPQGGSTSYAQKFQGRVTMTRDTSTSTVYMELSSLRSED
TAVYYCARETAYYTTKGNWFDPWGQGTLVTVSA
42Y073-1A01-191 variable light chain
nucleic acid sequence
SEQ ID NO: 83
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
AGCCTGTACCCCCCCAGGACTTTCGGCGGCGGCACCAAGGTGGAG
ATTAAG
42Y073-1A01-191 variable heavy chain
nucleic acid sequence
SEQ ID NO: 84
CAGGTGCAGCTGGTGCAGAGCGGCGCCGAAGTGAAAAAGCCCGGC
GCCAGCGTGAAGGTCAGCTGCAAGGCCTCCGGGTACACCTTCACC
AAGTACAGGATGCACTGGGTGAGGCAGGCCCCCGGCCAGGGCCTC
GAGTGGATGGGCATCATCAACCCCCAGGGAGGCAGCACCAGCTAC
GCCCAGAAGTTCCAGGGCAGGGTGACCATGACAAGGGACACCAGC
ACCAGCACCGTGTACATGGAGCTGAGCAGCCTGAGGAGCGAGGAC
ACCGCCGTGTATTACTGCGCAAGGGAGACCGCCTACTACACCACC
AAGGGCAACTGGTTCGACCCCTGGGGCCAGGGCACCCTGGTGACC
GTGAGCGCC
42Y073-86F08-66 variable light chain
amino acid sequence
SEQ ID NO: 85
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-66 variable heavy chain
amino acid sequence
SEQ ID NO: 86
QVQLVQSGAEVKKPGSSVKVSCKASGGTFVEYAISWVRQAPGQGL
EWMGGIIPAFGTAQYAQKFQGRVTITADESTSTAYMELSSLRSED
TAVYYCARGAGYYGDKDPMDVWGQGTTVTVSS
42Y073-86F08-66 variable light chain
nucleic acid sequence
SEQ ID NO: 87
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-66 variable heavy chain
nucleic acid sequence
SEQ ID NO: 88
CAGGTGCAGCTGGTGCAGAGCGGCGCAGAGGTGAAAAAGCCCGGC
AGCAGCGTGAAGGTGAGCTGCAAGGCCTCCGGCGGGACCTTCGTG
GAGTACGCCATCAGCTGGGTGAGGCAGGCTCCCGGACAGGGCCTG
GAGTGGATGGGCGGCATCATCCCCGCCTTCGGCACCGCCCAGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGAGCCGGCTACTACGGCGAC
AAGGACCCCATGGACGTGTGGGGCCAGGGCACCACCGTGACTGTG
AGCAGC
42Y073-86F08-16 variable light chain
amino acid sequence
SEQ ID NO: 89
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIK
42Y073-86F08-16 variable heavy chain
amino acid sequence
SEQ ID NO: 90
QVQLVQSGAEVKKPGSSVKVSCKASGGTFNEYAISWVRQAPGQGL
EWMGGIVPVFGTAKYAQKFQGRVTITADESTSTAYMELSSLRSED
TAVYYCARGAGYYGDKDPMDVWGQGTTVTVSS
42Y073-86F08-16 variable light chain
nucleic acid sequence
SEQ ID NO: 91
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAG
42Y073-86F08-16 variable heavy chain
nucleic acid sequence
SEQ ID NO: 92
CAGGTGCAGCTGGTGCAGAGCGGCGCCGAGGTGAAAAAGCCCGGC
AGCAGCGTGAAGGTGAGCTGCAAGGCCTCCGGGGGGACCTTCAAC
GAGTACGCCATCAGCTGGGTGAGGCAGGCTCCCGGACAGGGCCTG
GAGTGGATGGGCGGCATCGTGCCCGTGTTCGGCACCGCCAAGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGCGCCGGCTACTACGGCGAC
AAGGACCCCATGGACGTGTGGGGCCAGGGCACCACCGTGACTGTG
AGCAGC
42Y073-86F04-23 variable light chain
amino acid sequence
SEQ ID NO: 93
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIK
42Y073-86F04-23 variable heavy chain
amino acid sequence
SEQ ID NO: 94
QVQLVQSGAEVKKPGSSVKVSCKASGGTFSGYPISWVRQAPGQGL
EWMGGIIPIMGTARYAQKFQGRVTITADESTSTAYMELSSLRSED
TAVYYCARLLGESGMDVWGQGTTVTVSS
42Y073-86F04-23 variable light chain
nucleic acid sequence
SEQ ID NO: 95
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
CCCTACTTCAGCCCCCCCACTTTCGGCGGCGGCACCAAGGTGGAG
ATTAAG
42Y073-86F04-23 variable heavy chain
nucleic acid sequence
SEQ ID NO: 96
CAGGTGCAGCTGGTGCAGAGCGGCGCCGAGGTGAAAAAGCCCGGC
AGCAGCGTGAAGGTGAGCTGCAAGGCCTCCGGCGGGACCTTCAGC
GGCTACCCCATCAGCTGGGTGAGGCAGGCTCCCGGACAGGGCCTG
GAGTGGATGGGGGGCATCATCCCCATTATGGGCACCGCCAGGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGCTGCTGGGCGAGAGCGGCATG
GACGTGTGGGGCCAGGGCACCACCGTGACTGTGAGCAGC
CDRL1 amino acid sequence 1
SEQ ID NO: 97
RASQSISSYLN
CDRL1 amino acid sequence 2
SEQ ID NO: 98
RASQSVSSYLA
CDRL2 amino acid sequence 1
SEQ ID NO: 99
AASSLOS
CDRL2 amino acid sequence 2
SEQ ID NO: 100
DASKRAT
CDRL3 amino acid sequence 1
SEQ ID NO: 101
QQVLHTIT
CDRL3 amino acid sequence 2
SEQ ID NO: 102
QQPYFSPPT
CDRL3 amino acid sequence 3
SEQ ID NO: 103
QQLDNWPIT
CDRL3 amino acid sequence 4
SEQ ID NO: 104
QQSLYPPRT
CDRH1 amino acid sequence 1
SEQ ID NO: 105
SYAIS
CDRH1 amino acid sequence 2
SEQ ID NO: 106
YNAIS
CDRH1 amino acid sequence 3
SEQ ID NO: 107
SEAIS
CDRH1 amino acid sequence 4
SEQ ID NO: 108
SHAIS
CDRH1 amino acid sequence 5
SEQ ID NO: 109
GHAIS
CDRH1 amino acid sequence 6
SEQ ID NO: 110
SRAIS
CDRH1 amino acid sequence 7
SEQ ID NO: 111
SSAIS
CDRH1 amino acid sequence 8
SEQ ID NO: 112
NYAIS
CDRH1 amino acid sequence 9
SEQ ID NO: 113
EYAIH
CDRH1 amino acid sequence 10
SEQ ID NO: 114
LYAIS
CDRH1 amino acid sequence 11
SEQ ID NO: 115
EYAIS
CDRH1 amino acid sequence 12
SEQ ID NO: 116
SYAMH
CDRH1 amino acid sequence 13
SEQ ID NO: 117
QYAIHY
CDRH1 amino acid sequence 14
SEQ ID NO: 118
RYYWS
CDRH1 amino acid sequence 15
SEQ ID NO: 119
SYPMH
CDRH1 amino acid sequence 16
SEQ ID NO: 120
RAAMH
CDRH1 amino acid sequence 17
SEQ ID NO: 121
TYRMH
CDRH1 amino acid sequence 18
SEQ ID NO: 122
AYQMH
CDRH1 amino acid sequence 19
SEQ ID NO: 123
RYNMH
CDRH1 amino acid sequence 20
SEQ ID NO: 124
KYRMH
CDRH1 amino acid sequence 21
SEQ ID NO: 125
GYPIS
CDRH2 amino acid sequence 1
SEQ ID NO: 126
GIIPIFGTASYAQKFQG
CDRH2 amino acid sequence 2
SEQ ID NO: 127
GIIPIMGTARYAQKFQG
CDRH2 amino acid sequence 3
SEQ ID NO: 128
GIIPIFGRARYAQKFQG
CDRH2 amino acid sequence 4
SEQ ID NO: 129
GIIPIFGRGKYAQKFQG
CDRH2 amino acid sequence 5
SEQ ID NO: 130
GILPIFGRANYAQKFQG
CDRH2 amino acid sequence 6
SEQ ID NO: 131
GIIPIFGRANYAQKFQG
CDRH2 amino acid sequence 7
SEQ ID NO: 132
GIIPALGTANYAQKFQG
CDRH2 amino acid sequence 8
SEQ ID NO: 133
NIIPIFGTAGYAQKFQG
CDRH2 amino acid sequence 9
SEQ ID NO: 134
GIIPQLGTANYAQKFQG
CDRH2 amino acid sequence 10
SEQ ID NO: 135
GIIPVFGTANYAQKFQG
CDRH2 amino acid sequence 11
SEQ ID NO: 136
GIIPIFGTAWYYAQKFQG
CDRH2 amino acid sequence 12
SEQ ID NO: 137
VIIPIFGKANYYAQKFQG
CDRH2 amino acid sequence 13
SEQ ID NO: 138
EIDGWGSTNYNPSLKS
CDRH2 amino acid sequence 14
SEQ ID NO: 139
IINPSGGFTSYAQKFQG
CDRH2 amino acid sequence 15
SEQ ID NO: 140
IINPAGGYTSYAQKFQG
CDRH2 amino acid sequence 16
SEQ ID NO: 141
IINPSGGLTQYAQKFQG
CDRH2 amino acid sequence 17
SEQ ID NO: 142
IINPAGGWTSYAQKFQG
CDRH2 amino acid sequence 18
SEQ ID NO: 143
WINPAGGSTSYAQKFQG
CDRH2 amino acid sequence 19
SEQ ID NO: 144
IINPQGGSTSYAQKFQG
CDRH2 amino acid sequence 20
SEQ ID NO: 145
GIIPAFGTAQYAQKFQG
CDRH2 amino acid sequence 21
SEQ ID NO: 146
GIVPVFGTAKYAQKFQG
CDRH3 amino acid sequence 1
SEQ ID NO: 147
GAGYYGDKDPMDV
CDRH3 amino acid sequence 2
SEQ ID NO: 148
LLGESGMDV
CDRH3 amino acid sequence 3
SEQ ID NO: 149
GGSVDFWSGSDYYYYMDV
CDRH3 amino acid sequence 4
SEQ ID NO: 150
ETAYYTTKGNWFDP
PD-1 antagonist CDRH1
SEQ ID NO: 151
SYDMS
PD-1 antagonist CDRH2
SEQ ID NO: 152
TISGGGSYTYYQDSVKG
PD-1 antagonist CDRH3
SEQ ID NO: 153
PYYAMDY
PD-1 antagonist CDRL1
SEQ ID NO: 154
KASQDVGTAVA
PD-1 antagonist CDRL2
SEQ ID NO: 155
WASTLHT
PD-1 antagonist CDRL3
SEQ ID NO: 156
QHYSSYPWT
PD-1 antagonist alternative CDRL3
SEQ ID NO: 157
QHYNSYPWT
PD-1 antagonist heavy chain
variable region
SEQ ID NO: 158
EVQLLESGGGLVQPGGSLRLSCAASGFTFSSYDMSWVRQAPGKGL
EWVSTISGGGSYTYYQDSVKGRFTISRDNSKNTLYLQMNSLRAED
TAVYYCASPYYAMDYWGQGTTVTVSS
PD-1 antagonist light chain
variable region
SEQ ID NO: 159
DIQLTQSPSFLSAYVGDRVTITCKASQDVGTAVAWYQQKPGKAPK
LLIYWASTLHTGVPSRFSGSGSGTEFTLTISSLQPEDFATYYCQH
YSSYPWTFGQGTKLEIK
PD-1 antagonist monoclonal antibody
heavy chain
SEQ ID NO: 160
EVQLLESGGGLVQPGGSLRLSCAASGFTFSSYDMSWVRQAPGKGL
EWVSTISGGGSYTYYQDSVKGRFTISRDNSKNTLYLQMNSLRAED
TAVYYCASPYYAMDYWGQGTTVTVSSASTKGPSVFPLAPCSRSTS
ESTAALGCLVKDYFPEPVTVSWNSGALTSGVHTFPAVLQSSGLYS
LSSVVTVPSSSLGTKTYTCNVDHKPSNTKVDKRVESKYGPPCPPC
PAPEFLGGPSVFLFPPKPKDTLMISRTPEVTCVVVDVSQEDPEVQ
FNWYVDGVEVHNAKTKPREEQFNSTYRVVSVLTVLHQDWLNGKEY
KCKVSNKGLPSSIEKTISKAKGQPREPQVYTLPPSQEEMTKNQVS
LTCLVKGFYPSDIAVEWESNGQPENNYKTTPPVLDSDGSFFLYSR
LTVDKSRWQEGNVFSCSVMHEALHNHYTQKSLSLSLGK
PD-1 antagonist monoclonal antibody
light chain
SEQ ID NO: 161
DIQLTQSPSFLSAYVGDRVTITCKASQDVGTAVAWYQQKPGKAPK
LLIYWASTLHTGVPSRFSGSGSGTEFTLTISSLQPEDFATYYCQH
YSSYPWTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
PD-1 antagonist heavy chain
sequence
with N380D modification
SEQ NO: 162
EVQLLESGGGLVQPGGSLRLSCAASGFTFSSYDMSWVRQAPGKGL
EWVSTISGGGSYTYYQDSVKGRFTISRDNSKNTLYLQMNSLRAED
TAVYYCASPYYAMDYWGQGTTVTVSSASTKGPSVFPLAPCSRSTS
ESTAALGCLVKDYFPEPVTVSWNSGALTSGVHTFPAVLQSSGLYS
LSSVVTVPSSSLGTKTYTCNVDHKPSNTKVDKRVESKYGPPCPPC
PAPEFLGGPSVFLFPPKPKDTLMISRTPEVTCVVVDVSQEDPEVQ
FNWYVDGVEVHNAKTKPREEQFNSTYRVVSVLTVLHQDWLNGKEY
KCKVSNKGLPSSIEKTISKAKGQPREPQVYTLPPSQEEMTKNQVS
LTCLVKGFYPSDIAVEWESDGQPENNYKTTPPVLDSDGSFFLYSR
LTVDKSRWQEGNVFSCSVMHEALHNHYTQKSLSLSLGK
PD-1 antagonist light chain
sequence with N385D modification
SEQ NO: 163
EVQLLESGGGLVQPGGSLRLSCAASGFTFSSYDMSWVRQAPGKGL
EWVSTISGGGSYTYYQDSVKGRFTISRDNSKNTLYLQMNSLRAED
TAVYYCASPYYAMDYWGQGTTVTVSSASTKGPSVFPLAPCSRSTS
ESTAALGCLVKDYFPEPVTVSWNSGALTSGVHTFPAVLQSSGLYS
LSSVVTVPSSSLGTKTYTCNVDHKPSNTKVDKRVESKYGPPCPPC
PAPEFLGGPSVFLFPPKPKDTLMISRTPEVTCVVVDVSQEDPEVQ
FNWYVDGVEVHNAKTKPREEQFNSTYRVVSVLTVLHQDWLNGKEY
KCKVSNKGLPSSIEKTISKAKGQPREPQVYTLPPSQEEMTKNQVS
LTCLVKGFYPSDIAVEWESNGQPEDNYKTTPPVLDSDGSFFLYSR
LTVDKSRWQEGNVFSCSVMHEALHNHYTQKSLSLSLGK
PD-1 antagonist heavy chain
sequence with N380D and N385D modifications
SEQ NO: 164
EVQLLESGGGLVQPGGSLRLSCAASGFTFSSYDMSWVRQAPGKGL
EWVSTISGGGSYTYYQDSVKGRFTISRDNSKNTLYLQMNSLRAED
TAVYYCASPYYAMDYWGQGTTVTVSSASTKGPSVFPLAPCSRSTS
ESTAALGCLVKDYFPEPVTVSWNSGALTSGVHTFPAVLQSSGLYS
LSSVVTVPSSSLGTKTYTCNVDHKPSNTKVDKRVESKYGPPCPPC
PAPEFLGGPSVFLFPPKPKDTLMISRTPEVTCVVVDVSQEDPEVQ
FNWYVDGVEVHNAKTKPREEQFNSTYRVVSVLTVLHQDWLNGKEY
KCKVSNKGLPSSIEKTISKAKGQPREPQVYTLPPSQEEMTKNQVS
LTCLVKGFYPSDIAVEWESDGQPEDNYKTTPPVLDSDGSFFLYSR
LTVDKSRWQEGNVFSCSVMHEALHNHYTQKSLSLSLGK
42Y073-86F08-66 full length heavy chain
amino acid sequence
SEQ ID NO: 165
QVQLVQSGAEVKKPGSSVKVSCKASGGTFVEYAISWVRQAPGQGL
EWMGGIIPAFGTAQYAQKFQGRVTITADESTSTAYMELSSLRSED
TAVYYCARGAGYYGDKDPMDVWGQGTTVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK
42Y073-86F08-66 full length light chain
amino acid sequence
SEQ ID NO: 166
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC
42Y073-86F08-66 full length heavy chain
DNA sequence
SEQ ID NO: 167
CAGGTGCAGCTGGTGCAGAGCGGCGCAGAGGTGAAAAAGCCCGGC
AGCAGCGTGAAGGTGAGCTGCAAGGCCTCCGGCGGGACCTTCGTG
GAGTACGCCATCAGCTGGGTGAGGCAGGCTCCCGGACAGGGCCTG
GAGTGGATGGGCGGCATCATCCCCGCCTTCGGCACCGCCCAGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGGGAGCCGGCTACTACGGCGAC
AAGGACCCCATGGACGTGTGGGGCCAGGGCACCACCGTGACTGTG
AGCAGCGCCAGCACCAAGGGCCCCAGCGTGTTCCCCCTGGCCCCC
AGCAGCAAGAGCACCAGCGGCGGCACAGCCGCCCTGGGCTGCCTG
GTGAAGGACTACTTCCCCGAGCCCGTGACCGTGTCCTGGAACAGC
GGAGCCCTGACCAGCGGCGTGCACACCTTCCCCGCCGTGCTGCAG
AGCAGCGGCCTGTACAGCCTGAGCAGCGTGGTGACCGTGCCCAGC
AGCAGCCTGGGCACCCAGACCTACATCTGTAACGTGAACCACAAG
CCCAGCAACACCAAGGTGGACAAGAAGGTGGAGCCCAAGAGCTGT
GACAAGACCCACACCTGCCCCCCCTGCCCTGCCCCCGAGCTGCTG
GGAGGCCCCAGCGTGTTCCTGTTCCCCCCCAAGCCTAAGGACACC
CTGATGATCAGCAGAACCCCCGAGGTGACCTGTGTGGTGGTGGAT
GTGAGCCACGAGGACCCTGAGGTGAAGTTCAACTGGTACGTGGAC
GGCGTGGAGGTGCACAATGCCAAGACCAAGCCCAGGGAGGAGCAG
TACAACAGCACCTACCGGGTGGTGTCCGTGCTGACCGTGCTGCAC
CAGGATTGGCTGAACGGCAAGGAGTACAAGTGTAAGGTGTCCAAC
AAGGCCCTGCCTGCCCCTATCGAGAAAACCATCAGCAAGGCCAAG
GGCCAGCCCAGAGAGCCCCAGGTGTACACCCTGCCCCCTAGCAGA
GATGAGCTGACCAAGAACCAGGTGTCCCTGACCTGCCTGGTGAAG
GGCTTCTACCCCAGCGACATCGCCGTGGAGTGGGAGAGCAACGGC
CAGCCCGAGAACAACTACAAGACCACCCCCCCTGTGCTGGACAGC
GATGGCAGCTTCTTCCTGTACAGCAAGCTGACCGTGGACAAGAGC
AGATGGCAGCAGGGCAACGTGTTCAGCTGCTCCGTGATGCACGAG
GCCCTGCACAATCACTACACCCAGAAGAGCCTGAGCCTGTCCCCT
GGCAAG
42Y073-86F08-66 full length light chain
DNA sequence
SEQ ID NO: 168
GACATCCAGCTGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGCGGCGTGCCCTCT
AGGTTTAGCGGCAGCGGCAGCGGCACCGACTTCACCCTGACCATC
AGCAGCCTCCAGCCCGAGGACTTCGCCACCTACTACTGCCAGCAG
GTGCTGCACACCATCACTTTCGGCGGCGGCACCAAGGTGGAGATT
AAGCGTACGGTGGCCGCCCCCAGCGTGTTCATCTTCCCCCCCAGC
GATGAGCAGCTGAAGAGCGGCACCGCCAGCGTGGTGTGTCTGCTG
AACAACTTCTACCCCCGGGAGGCCAAGGTGCAGTGGAAGGTGGAC
AATGCCCTGCAGAGCGGCAACAGCCAGGAGAGCGTGACCGAGCAG
GACAGCAAGGACTCCACCTACAGCCTGAGCAGCACCCTGACCCTG
AGCAAGGCCGACTACGAGAAGCACAAGGTGTACGCCTGTGAGGTG
ACCCACCAGGGCCTGTCCAGCCCCGTGACCAAGAGCTTCAACCGG
GGCGAGTGC
42Y073-86F08-16 full length light chain
amino acid sequence
SEQ ID NO: 169
DIQLTQSPSSLSASVGDRVTITCRASQSISSYLNWYQQKPGKAPK
LLIYAASSLQSGVPSRFSGSGSGTDFTLTISSLQPEDFATYYCQQ
VLHTITFGGGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSFNRGEC
42Y073-86F08-16 full length heavy chain
amino acid sequence
SEQ ID NO: 170
QVQLVQSGAEVKKPGSSVKVSCKASGGTFNEYAISWVRQAPGQGL
EWMGGIVPVEGTAKYAQKFQGRVTITADESTSTAYMELSSLRSED
TAVYYCARGAGYYGDKDPMDVWGQGTTVTVSSASTKGPSVFPLAP
SSKSTSGGTAALGQLVKDYFPEPVTVSWNSGALTSGVHTFPAVLQ
SSGLYSLSSVVTVPSSSLGTQTYICNVNHKPSNTKVDKKVEPKSC
DKTHTQPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK
42Y073-86F08-16 full length light chain
DNA sequence
SEQ ID NO: 171
GACATCCAGCTGACCCAGAGCCCTAGCAGCCTGAGCGCCAGCGTG
GGAGACAGGGTGACCATCACCTGCAGGGCCAGCCAGTCCATCAGC
AGGTACCTGAACTGGTACCAGCAGAAGCCCGGCAAGGCCCCCAAG
GTGGTGATGTACGCCGCAAGCTCACTGCAGAGGGGGGTGCCCTCT
AGGTTTAGCGGCAGCGGCAGCGGCACCGACTTCACCCTGACCATC
AGCAGGGTGCAGCCCGAGGACTTCGCCACCTACTACTGCCAGCAG
GTGCTGCACACCATGACTTTCGGGGGGGGCACCAAGGTGGAGATT
AAGCGTACGGTGGGCGCCCCCAGGGTGTTCATCTTCCCCCCCAGG
GATGAGCAGGTGAAGAGCGGCACCGCCAGCGTGGTGTGTCTGCTG
AACAACTTCTACCCCCGGGAGGCCAAGGTGCAGTGGAAGGTGGAG
AATGCCCTGCAGAGCGGCAACAGCCAGGAGAGCGTGACCGAGCAG
GACAGCAAGGACTCCACCTACAGCCTGAGCAGCACCCTGACCGTG
AGGAAGGCCGACTACGAGAAGCACAAGGTGTACGCCTGTGAGGTG
ACCCACCAGGGCCTGTCCAGCCCCGTGACCAAGAGCTTCAACGGG
GGGGAGTGC
42Y073-86F08-16 full length heavy chain
DNA sequence
SEQ ID NO: 172
CAGGTGCAGCTGGTGCAGAGGGGCGCCGAGGTGAAAAAGCCCGGC
AGCAGCGTGAAGGTGAGCTGCAAGGCCTCCGGGGGGACCTTCAAC
GAGTACGCCATCAGGTGGGTGAGGCAGGGTCCCGGACAGGGCCTG
GAGTGGATGGGGGGCATCGTGCCCGTGTTCGGCACCGCCAAGTAC
GCGCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGGGCCAGGGGGGCCGGCTACTACGGCGAG
AAGGACCCCATGGACGTGTGGGGCCAGGGCACCACCGTGACTGTG
AGCAGGGCCAGCACCAAGGGCCCCAGCGTGTTCCCCCTGGCCCCC
AGCAGCAAGAGCACCAGGGGGGGCACAGCCGCCCTGGGCTGCCTG
GTGAAGGACTACTTCCCCGAGCCCGTGACCGTGTCCTGGAACAGC
GGAGCCCTGACCAGCGGCGTGCACACGTTGCCCGCGGTGCTGCAG
AGCAGCGGCCTGTACAGCGTGAGCAGCGTGGTGACCGTGCCCAGC
AGCAGCCTGGGCACCCAGACCTACATCTGTAACGTGAACCACAAG
CCCAGCAACACCAAGGTGGACAAGAAGGTGGAGCCCAAGAGCTGT
GACAAGACCCACACCTGCCCCCCCTGCCCTGCGCCCGAGCTGCTG
GGAGGCCCCAGCGTGTTCCTGTTCCCCCCCAAGCCTAAGGACACC
CTGATGATCAGCAGAACCCCCGAGGTGACCTGTGTGGTGGTGGAT
GTGAGCCACGAGGACCCTGAGGTGAAGTTCAACTGGTACGTGGAC
GGCGTGGAGGTGCACAATGCCAAGACCAAGCCCAGGGAGGAGCAG
TACAACAGCACCTACCGGGTGGTGTCCGTGCTGACCGTGCTGCAC
CAGGATTGGCTGAACGGCAAGGAGTACAAGTGTAAGGTGTCCAAC
AAGGCCCTGCCTGCCCCTATCGAGAAAACCATCAGCAAGGCCAAG
GGCCAGCCCAGAGAGCCCCAGGTGTACACCCTGCCCCCTAGCAGA
GATGAGCTGACCAAGAACCAGGTGTCCCTGACCTGCCTGGTGAAG
GGCTTCTACCCCAGCGACATCGCCGTGGAGTGGGAGAGGAACGGC
CAGCCCGAGAACAACTACAAGACCACCCCCCGTGTGGTGGACAGC
GATGGGAGCTTCTTCCTGTACAGCAAGCTGACCGTGGACAAGAGC
AGATGGCAGCAGGGCAACGTGTTCAGCTGCTCCGTGATGCACGAG
GCCCTGCACAATCACTACACCCAGAAGAGCGTGAGCCTGTCCGGT
GGCAAG
42Y073-86F04-23 full length light chain
amino acid sequence
SEQ ID NO: 173
DIQMTQSPSSLSASVGDRVTITCRASQSISSYLNWYQQKPGKAPK
LLIYAASSLQSGVPSRFSGSGSGTDFTLTISSLQPEDFATYYCQQ
PYFSPPTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
42Y073-86F04-23 full length Heavy chain
amino acid sequence
SEQ ID NO: 174
QVQLVQSGAEVKKPGSSVKVSCKASGGTFSGYPISWVRQAPGQGL
EWMGGIIPIMGTARYAQKFQGRVTITADESTSTAYMELSSLRSED
TAVYYCARLLGESGMDVWGQGTTVTVSSASTKGPSVFPLAPSSKS
TSGGTAALGQLVKDYFPEPVTVSWNSGALTSGVHTFPAVLQSSGL
YSLSSVVTVPSSSLGTQTYICNVNHKPSNTKVDKKVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSRDELT
KNQVSLTOLVKGFYPSDIAVEWESNGQPENNYKTTPPVLDSDGSF
FLYSKLTVDKSRWQQGNVFSCSVMHEALHNHYTQKSLSLSPGK
42Y073-86F04-23 full length Light chain
DNA sequence
SEQ ID NO: 175
GACATCCAGATGACCCAGAGCCCTAGCAGCCTGAGCGCCAGCGTG
GGAGACAGGGTGACCATCACCTGCAGGGCCAGCCAGTCCATCAGC
AGCTACCTGAACTGGTACCAGCAGAAGCCCGGCAAGGCCCCCAAG
CTGCTGATCTACGCCGCAAGCTCACTGCAGAGGGGGGTGCCCTCT
AGGTTTAGCGGCAGCGGCAGGGGCACCGACTTCACCCTGACCATC
AGCAGCCTCCAGCCCGAGGACTTCGCCACCTACTACTGCCAGCAG
CCCTACTTCAGCCCCCCCACTTTCGGGGGGGGCACCAAGGTGGAG
ATTAAGCGTACGGTGGCCGCCCCCAGCGTGTTCATCTTCCCCCCC
AGCGATGAGCAGCTGAAGAGCGGCACCGCCAGCGTGGTGTGTCTG
CTGAACAACTTCTACCCCCGGGAGGCCAAGGTGCAGTGGAAGGTG
GACAATGCCCTGCAGAGCGGCAACAGCCAGGAGAGCGTGACCGAG
CAGGACAGCAAGGACTCCACCTACAGCCTGAGCAGCACCCTGACC
CTGAGCAAGGCCGACTACGAGAAGCACAAGGTGTACGCCTGTGAG
GTGACCCACCAGGGCCTGTCCAGCCCCGTGACCAAGAGCTTCAAC
CGGGGCGAGTGC
42Y073-86F04-23 full length Heavy chain
DNA sequence
SEQ ID NO: 176
CAGGTGCAGCTGGTGCAGAGGGGCGCCGAGGTGAAAAAGCCCGGC
AGCAGCGTGAAGGTGAGCTGCAAGGCCTCCGGGGGGACCTTCAGG
GGCTACCCCATCAGCTGGGTGAGGCAGGCTCCCGGACAGGGCCTG
GAGTGGATGGGGGGCATCATCCCCATTATGGGCACCGCCAGGTAC
GCCCAGAAGTTCCAGGGAAGGGTCACCATCACCGCCGACGAGAGC
ACCAGCACCGCCTACATGGAACTCAGCAGCCTGAGGAGCGAGGAC
ACCGCCGTGTACTATTGCGCCAGGCTGCTGGGCGAGAGCGGCATG
GACGTGTGGGGCCAGGGCACCACCGTGACTGTGAGCAGCGCCAGC
ACCAAGGGCCCCAGCGTGTTCCCCCTGGCCCCCAGCAGCAAGAGC
ACCAGCGGCGGCACAGCCGCCCTGGGCTGCCTGGTGAAGGACTAC
TTCCCCGAGCCCGTGACCGTGTCCTGGAACAGCGGAGCCCTGACC
AGCGGCGTGCACACCTTCCCCGCCGTGCTGCAGAGCAGCGGCCTG
TACAGCCTGAGCAGCGTGGTGACCGTGCCCAGCAGCAGCCTGGGC
ACCCAGACCTACATCTGTAACGTGAACCACAAGCCCAGCAACACC
AAGGTGGACAAGAAGGTGGAGCCCAAGAGCTGTGAGAAGACCCAC
ACCTGCCCCCCCTGCCCTGCCCCCGAGCTGCTGGGAGGCCCCAGC
GTGTTCCTGTTCCCCCCCAAGCCTAAGGACACCCTGATGATCAGC
AGAACCCCCGAGGTGACCTGTGTGGTGGTGGATGTGAGCCACGAG
GACCCTGAGGTGAAGTTCAACTGGTACGTGGAGGGGGTGGAGGTG
CACAATGCCAAGACCAAGCCCAGGGAGGAGCAGTACAACAGCACC
TACCGGGTGGTGTCCGTGCTGACCGTGCTGCACCAGGATTGGCTG
AACGGCAAGGAGTACAAGTGTAAGGTGTCCAACAAGGCCCTGCCT
GCCCCTATCGAGAAAACCATCAGCAAGGCCAAGGGCCAGCCCAGA
GAGCCCCAGGTGTACACCCTGCCCCCTAGCAGAGATGAGCTGACC
AAGAACCAGGTGTCCCTGACCTGCCTGGTGAAGGGCTTCTACCCC
AGCGACATCGCCGTGGAGTGGGAGAGCAACGGCCAGCCCGAGAAC
AACTACAAGACCACCCCCCCTGTGCTGGACAGCGATGGCAGCTTC
TTCCTGTAGAGCAAGCTGACCGTGGACAAGAGCAGATGGCAGCAG
GGCAACGTGTTCAGCTGCTCCGTGATGCACGAGGCCCTGCACAAT
CACTACACCCAGAAGAGCCTGAGCCTGTCCCCTGGCAAG
CD155-fc amino acid sequence used in examples
(sequence in bold type is the mature CD155
ECD (extracellular domain)
without leader sequence,
the remainder is the human IgG1 Fc
region)
SEQ ID NO: 177
WPPPGTGDVVVQAPTQVPGFLGDSVTLPCYLQVPNMEVTHVSQLT
WARHGESGSMAVFHQTQGPSYSESKRLEFVAARLGAELRNASLRM
FGLRVEDEGNYTCLFVTFPQGSRSVDIWLRVLAKPQNTAEVQKVQ
LTGEPVPMARCVSTGGRPPAQITWHSDLGGMPNTSQVPGFLSGTV
TVTSLWILVPSSQVDGKNVTCKVEHESFEKPQLLTVNLTVYYPPE
VSISGYDNNWYLGQNEATLTCDARSNPEPTGYNWSTTMGPLPPFA
VAQGAQLLIRPVDKPINTTLICNVTNALGARQAELTVQVKEGPPS
EHSGISRNSGENLYFQGDPKSCDKTHTCPPCPAPELLGGPSVFLF
PPKPKDTLMISRTPEVTCVVVDVSHEDPEVKFNWYVDGVEVHNAK
TKPREEQYNSTYRVVSVLTVLHQDWLNGKEYKCKVSNKALPAPIE
KTISKAKGQPREPQVYTLPPSRDELTKNQVSLTCLVKGFYPSDIA
VEWESNGQPENNYKTTPPVLDSDGSFFLYSKLTVDKSRWQQGNVF
SCSVMHEALHNHYTQKSLSLSPGK
CD155 amino acid sequence
(native leader sequence
is underlined and transmembrane and
cytoplasmic portion is in bold type)
SEQ ID NO: 178
MARAMAAAWPLLLVALLVLSWPPPGTGDVVVQAPTQVPGFLGDSV
TLPCYLQVPNMEVTHVSQLTWARHGESGSMAVFHQTQGPSYSESK
RLEFVAARLGAELRNASLRMFGLRVEDEGNYTCLFVTFPQGSRSV
DIWLRVLAKPQNTAEVQKVQLTGEPVPMARCVSTGGRPPAQITWH
SDLGGMPNTSQVPGFLSGTVTVTSLWILVPSSQVDGKNVTCKVEH
ESFEKPQLLTVNLTVYYPPEVSISGYDNNWYLGQNEATLTCDARS
NPEPTGYNWSTTMGPLPPFAVAQGAQLLIRPVDKPINTTLICNVT
NALGARQAELTVQVKEGPPSEHSGISRNAIIFLVLGILVFLILLG
IGIYFYWSKCSREVLWHCHLCPSSTEHASASANGHVSYSAVSREN
SSSQDPQTEGTR
Human IgG1 Fc region amino acid sequence
SEQ ID NO: 179
ASTKGPSVFPLAPSSKSTSGGTAALGQLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human IgG1 Fc region nucleic acid sequence
SEQ ID NO: 180
GCCAGCACCAAGGGCCCCAGCGTGTTCCCCCTGGCCCCCAGCAGC
AAGAGCACCAGCGGGGGCACAGCCGCCCTGGGCTGCCTGGTGAAG
GACTACTTCCCCGAGCCCGTGACCGTGTCCTGGAACAGCGGAGCC
CTGACCAGCGGCGTGCACACCTTCCCCGCCGTGCTGCAGAGCAGC
GGCCTGTACAGCCTGAGCAGCGTGGTGACCGTGCCCAGCAGCAGC
CTGGGCACCCAGACCTACATCTGTAACGTGAACCACAAGCCCAGC
AACACCAAGGTGGACAAGAAGGTGGAGCCCAAGAGCTGTGAGAAG
ACCCACACCTGCCCCCCCTGCCCTGCCCCCGAGCTGCTGGGAGGC
CCCAGCGTGTTCCTGTTCCCCCCCAAGCCTAAGGACACCCTGATG
ATCAGCAGAACCCCCGAGGTGACCTGTGTGGTGGTGGATGTGAGC
CACGAGGACCCTGAGGTGAAGTTCAACTGGTACGTGGACGGCGTG
GAGGTGCACAATGCCAAGACCAAGCCCAGGGAGGAGCAGTACAAC
AGCACCTACCGGGTGGTGTCCGTGCTGACCGTGCTGCACCAGGAT
TGGCTGAACGGCAAGGAGTACAAGTGTAAGGTGTCCAACAAGGCC
CTGCCTGCCCCTATCGAGAAAACCATCAGCAAGGCCAAGGGCCAG
CCCAGAGAGCCCCAGGTGTACACCCTGCCCCCTAGCAGAGATGAG
CTGACCAAGAACCAGGTGTCCCTGACCTGCCTGGTGAAGGGCTTC
TACCCCAGCGACATCGCCGTGGAGTGGGAGAGCAACGGCCAGCCC
GAGAACAACTACAAGACCACCCCCCCTGTGCTGGACAGCGATGGC
AGCTTCTTCCTGTACAGCAAGCTGACCGTGGACAAGAGCAGATGG
CAGCAGGGCAACGTGTTCAGCTGCTCCGTGATGCACGAGGCCCTG
CACAATCACTACACCCAGAAGAGCCTGAGCCTGTCCCCTGGCAAG

| SEQ ID | |
|---|---|
| NO | Sequence |
| 181 | FTFSEYTMN |
| 182 | AIVGSGDSTYYADSVKG |
| 183 | AKDYSSGDWIDYGMDV |
| 184 | QASQDISNYLN |
| 185 | DASNLAT |
| 186 | QQFDLLPPT |
| 187 | FTFSDYAMI |
| 188 | AISGGGESTYYADSVKG |
| 189 | AKDYSSGDWIDYGMDV |
| 190 | QASQDISNYLN |
| 191 | DASNLAT |
| 192 | QQFDLLPPT |
| 193 | GTFDNYYIS |
| 194 | GIFPIFGTANYAQKFQG |
| 195 | AREVGHYSGSPYYMDV |
| 196 | RASQSINSWLA |
| 197 | DASSLES |
| 198 | QQVGPYLT |
| 199 | FTFGDYAMS |
| 200 | FIGSKFYGGETEYTASVKG |
| 201 | ARGPRRYTYGMDV |
| 202 | RASQSISSYLN |
| 203 | AASSLQS |
| 204 | QQSSTPLT |
| 205 | FTFDDYAVH |
| 206 | GISWSSGLIGYADSVKG |
| 207 | AKGPPTYQDYFDL |
| 208 | RASQSVSRYLA |
| 209 | DASNRAT |
| 210 | QQVSFFPPIT |
| 211 | FTFSGHLMS |
| 212 | AISGSAGETYYADSVKG |
| 213 | ARDAYYDDWSGWADWYFDL |
| 214 | RASQSVSRYLA |
| 215 | DASNRAT |
| 216 | QQVSLLPPT |
| 217 | FTFGDVAMS |
| 218 | YIGSKAYGGETEYTASVKG |
| 219 | ARAGHSYGSIASNWFDP |
| 220 | RASQSISSYLN |
| 221 | GASSLQS |
| 222 | QQGFYTPWT |
| 223 | GTFSSAAIS |
| 224 | NIIPIVGIANYAQKFQG |
| 225 | ARDTGRGYTRHFWFDP |
| 226 | RASQSISSYLN |
| 227 | AASSLQS |
| 228 | QQSDILYT |
| 229 | GTFSNYAIS |
| 230 | GIIPIFGTANYAQKFQG |
| 231 | ARGRGALALVGPYYGMDV |
| 232 | RSSQSLLHSNGYNYLD |
| 233 | LGSHRAS |
| 234 | MQALRAPT |
| 235 | FTFGDYAMS |
| 236 | FIGSKAYGGTTEYTASVKG |
| 237 | ARGPRRYTYGMDV |
| 238 | RASQSISSYLN |
| 239 | AASSLQS |
| 240 | QQSSTPLT |
| 241 | GTFSSYAIS |
| 242 | GIIPISGTANYAQKFQG |
| 243 | ARDTGRGYTRHFWFDP |
| 244 | RASQSISSYLN |
| 245 | AASSLQS |
| 246 | QQSDILYT |
| 247 | GTFGNYAIS |
| 248 | GIIPIPGIANYAQKFQG |
| 249 | ARDTGRGYTRHFWFDP |
| 250 | RASQSISSYLN |
| 251 | AASSLQS |
| 252 | QQSDILYT |
| 253 | GTFSSAAIS |
| 254 | GIFPISGHANYAQKFQG |
| 255 | ARDTGRGYTRHFWFDP |
| 256 | RASQSISSYLN |
| 257 | AASSLQS |
| 258 | QQSDILYT |
| 259 | GTFATYAIS |
| 260 | GIFPLSGTANYAQKFQG |
| 261 | ARDTGRGYTRHFWFDP |
| 262 | RASQSISSYLN |
| 263 | AASSLQS |
| 264 | QQSDILYT |
| 265 | EVQLVESGGGLVKPGGSLRLSCAASGFTFS |
| | EYTMNWVRQAPGKGLEWVSAIVGSGDSTYY |
| | ADSVKGRFTISRDNSKNTLYLQMNSLRAED |
| | TAVYYCAKDYSSGDWIDYGMDVWGQGTTVT |
| | VSS |
| 266 | DIQMTQSPSSLSASVGDRVTITCQASQDIS |
| | NYLNWYQQKPGKAPKLLIYDASNLATGVPS |
| | RFSGSGSGTDFTFTISSLQPEDIATYYCQQ |
| | FDLLPPTFGGGTKVEIK |
| 267 | EVQLLESGGGLVQPGGSLRLSCAASGFTFS |
| | DYAMIWVRQAPGKGLEWVSAISGGGESTYY |
| | ADSVKGRFTISRDNSKNTLYLQMNSLRAED |
| | TAVYYCAKDYSSGDWIDYGMDVWGQGTTVT |
| | VSS |
| 268 | DIQMTQSPSSLSASVGDRVTITCQASQDIS |
| | NYLNWYQQKPGKAPKLLIYDASNLATGVPS |
| | RFSGSGSGTDFTFTISSLQPEDIATYYCQQ |
| | FDLLPPTFGGGTKVEIK |
| 269 | QVQLVQSGAEVKKPGSSVKVSCKASGGTFD |
| | NYYISWVRQAPGQGLEWMGGIFPIFGTANY |
| | AQKFQGRVTITADESTSTAYMELSSLRSED |
| | TAVYYCAREVGHYSGSPYYMDVWGKGTTVT |
| | VSS |
| 270 | DIQMTQSPSTLSASVGDRVTITCRASQSIN |
| | SWLAWYQQKPGKAPKLLISDASSLESGVPS |
| | RFSGSGSGTEFTLTISSLQPDDFATYYCQQ |
| | VGPYLTFGGGTKVEIK |
| 271 | EVQLVESGGGLVQPGRSLRLSCTASGFTFG |
| | DYAMSWFRQAPGKGLEWVGFIGSKFYGGET |
| | EYTASVKGRFTISRDGSKSIAYLQMNSLKT |
| | EDTAVYYCARGPRRYTYGMDVWGQGTTVTV |
| | SS |
| 272 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SSTPLTFGGGTKVEIK |
| 273 | EVQLVESGGGLVQPGRSLRLSCAASGFTFD |
| | DYAVHWVRQAPGKGLEWVSGISWSSGLIGY |
| | ADSVKGRFTISRDNAKNSLYLQMNSLRAED |
| | TAVYYCAKGPPTYQDYFDLWGRGTLVTVSS |
| 274 | EIVLTQSPATLSLSPGERATLSCRASQSVS |
| | RYLAWYQQKPGQAPRLLIYDASNRATGIPA |
| | RFSGSGSGTDFTLTISSLEPEDFAVYYCQQ |
| | VSFFPPITFGGGTKVEIK |
| 275 | EVQLLESGGGLVQPGGSLRLSCAASGFTFS |
| | GHLMSWVRQAPGKGLEWVSAISGSAGETYY |
| | ADSVKGRFTISRDNSKNTLYLQMNSLRAED |
| | TAVYYCARDAYYDDWSGWADWYFDLWGRGT |
| | LVTVSS |
| 276 | EIVLTQSPATLSLSPGERATLSCRASQSVS |
| | RYLAWYQQKPGQAPRLLIYDASNRATGIPA |
| | RFSGSGSGTDFTLTISSLEPEDFAVYYCQQ |
| | VSLLPPTFGGGTKVEIK |
| 277 | EVQLVESGGGLVQPGRSLRLSCTASGFTFG |
| | DVAMSWFRQAPGKGLEWVGYIGSKAYGGET |
| | EYTASVKGRFTISRDGSKSIAYLQMNSLKT |
| | EDTAVYYCARAGHSYGSIASNWFDPWGQGT |
| | LVTVSS |
| 278 | DIQMTQSPSSLSASVGDRVTITCRASQSIS |
| | SYLNWYQQKPGKAPKLLIYGASSLQSGVPS |
| | RFSGSGSGTDFTLTISSLQPEDFATYYCQQ |
| | GFYTPWTFGGGTKVEIK |
| 279 | QVQLVQSGAEVKKPGSSVKVSCKASGGTFS |
| | SAAISWVRQAPGQGLEWMGNIIPIVGIANY |
| | AQKFQGRVTITADESTSTAYMELSSLRSED |
| | TAVYYCARDTGRGYTRHFWFDPWGQGTLVT |
| | VSS |
| 280 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SDILYTFGGGTKVEIK |
| 281 | EVQLVQSGAEVKKPGSSVKVSCKASGGTFS |
| | NYAISWVRQAPGQGLEWMGGIIPIFGTANY |
| | AQKFQGRVTITADESTSTAYMELSSLRSED |
| | TAVYYCARGRGALALVGPYYGMDVWGQGTT |
| | VTVSS |
| 282 | DIVMTQSPLSLPVTPGEPASISCRSSQSLL |
| | HSNGYNYLDWYLQKPGQSPQLLIYLGSHRA |
| | SGVPDRFSGSGSGTDFTLKISRVEAEDVGV |
| | YYCMQALRAPTFGGGTKVEIK |
| 283 | EVQLVESGGGLVQPGRSLRLSCTASGFTFG |
| | DYAMSWFRQAPGKGLEWVGFIGSKAYGGTT |
| | EYTASVKGRFTISRDGSKSIAYLQMNSLKT |
| | EDTAVYYCARGPRRYTYGMDVWGQGTTVTV |
| | SS |
| 284 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SSTPLTFGGGTKVEIK |
| 285 | QVQLVQSGAEVKKPGSSVKVSCKASGGTFS |
| | SYAISWVRQAPGQGLEWMGGIIPISGTANY |
| | AQKFQGRVTITADESTSTAYMELSSLRSED |
| | TAVYYCARDTGRGYTRHFWFDPWGQGTLVT |
| | VSS |
| 286 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SDILYTFGGGTKVEIK |
| 287 | QVQLVQSGAEVKKPGSSVKVSCKASGGTFG |
| | NYAISWVRQAPGQGLEWMGGIIPIPGIANY |
| | AQKFQGRVTITADESTSTAYMELSSLRSED |
| | TAVYYCARDTGRGYTRHFWFDPWGQGTLVT |
| | VSS |
| 288 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SDILYTFGGGTKVEIK |
| 289 | QVQLVQSGAEVKKPGSSVKVSCKASGGTFS |
| | SAAISWVRQAPGQGLEWMGGIFPISGHANY |
| | AQKFQGRVTITADESTSTAYMELSSLRSED |
| | TAVYYCARDTGRGYTRHFWFDPWGQGTLVT |
| | VSS |
| 290 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SDILYTFGGGTKVEIK |
| 291 | QVQLVQSGAEVKKPGSSVKVSCKASGGTFA |
| | TYAISWVRQAPGQGLEWMGGIFPLSGTANY |
| | AQKFQGRVTITADESTSTAYMELSSLRSED |
| | TAVYYCARDTGRGYTRHFWFDPWGQGTLVT |
| | VSS |
| 292 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SDILYTFGGGTKVEIK |
| 293 | GYTFTDYN |
| 294 | TYPYIGGS |
| 295 | AREDKTARNAMDY |
| 296 | ENIYSN |
| 297 | EAT |
| 298 | QHFWGTPYT |
| 299 | QVQLVQSGAEVKKPGASVKVSCKASGYTFT |
| | DYNINWVRQAPGQGLEWMGYIYPYIGGSGY |
| | AQKFQGRVTMTRDTSTSTVYMELSSLRSED |
| | TAVYYCAREDKTARNAMDYWGQGTLVTVSS |
| 300 | DIQMTQSPSSLSASVGDRVTITCRVSENIY |
| | SNLAWYQQKPGKAPKLLIYEATNLAEGVPS |
| | RFSGSGSGTDFTLTISSLQPEDFATYYCQH |
| | FWGTPYTFGQGTKLEIK |
| 301 | QVQLVQSGAEVKKPGASVKVSCKASGYAFTN |
| | YLIEWVRQAPGQGLEWIGVINPGSGGIYYA |
| | QKFQGRVTMTADTSTSTVYMELSSLRSEDT |
| | AVYYCARSETHDTWFAYWGQGTLVTVSS |
| 302 | DIQMTQSPSSLSASVGDRVTITCKASQSVR |
| | IAVAWFQQKPGKAPKALIYLASTRHTGVPS |
| | RFSGSGSGTDFTLTISSVQPEDFATYYCLQ |
| | HWNYPYTFGQGTKLEIK |
| 303 | GTFSSAAIS |
| 304 | NIIPIVGIANYAQKFQG |
| 305 | ARDTGRGYTRHFWFDP |
| 306 | RASQSISSYLN |
| 307 | AASSLQS |
| 308 | QQSDILYT |
| 309 | QVQLVQSGAEVKKPGSSVKVSCKASGGTFS |
| | SAAISWVRQAPGQGLEWMGNIIPIVGIANY |
| | AQKFQGRVTITADESTSTAYMELSSLRSED |
| | TAVYYCARDTGRGYTRHFWFDPWGQGTLVT |
| | VSS |
| 310 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SDILYTFGGGTKVEIK |
| 311 | DIVMTQSPDSLAVSLGERATINCKSSQTVL |
| | YSSNNKKYLAWYQQKPGQPPNLLIYWASTR |
| | ESGVPDRFSGSGSGTDFTLTISSLQAEDVA |
| | VYYCQQYYSTPFTFGPGTKVEIKRTVAAPS |
| | VFIFPPSDEQLKSGTASVVCLLNNFYPREA |
| | KVQWKVDNALQSGNSQESVTEQDSKDSTYS |
| | LSSTLTLSKADYEKHKVYACEVTHQGLSSP |
| | VTKSFNRGEC |
| 312 | EVQLQQSGPGLVKPSQTLSLTCAISGDSVS |
| | SNSAAWNWIRQSPSRGLEWLGKTYYRFKWY |
| | SDYAVSVKGRITINPDTSKNQFSLQLNSVT |
| | PEDTAVFYCTRESTTYDLLAGPFDYWGQGT |
| | LVTVSSASTKGPSVFPLAPSSKSTSGGTAA |
| | LGCLVKDYFPEPVTVSWNSGALTSGVHTFP |
| | AVLQSSGLYSLSSVVTVPSSSLGTQTYICN |
| | VNHKPSNTKVDKKVEPKSCDKTHTCPPCPA |
| | PELLGGPSVFLFPPKPKDTLMISRTPEVTC |
| | VVVDVSHEDPEVKFNWYVDGVEVHNAKTKP |
| | REEQYNSTYRVVSVLTVLHQDWLNGKEYKC |
| | KVSNKALPAPIEKTISKAKGQPREPQVYTL |
| | PPSREEMTKNQVSLTCLVKGFYPSDIAVEW |
| | ESNGQPENNYKTTPPVLDSDGSFFLYSKLT |
| | VDKSRWQQGNVFSCSVMHEALHNHYTQKSL |
| | SLSPGK |
| 313 | DIVMTQSPSSLAVSPGEKVTMTCKSSQSLY |
| | YSGVKENLLAWYQQKPGQSPKLLIYYASIR |
| | FTGVPDRFTGSGSGTDYTLTITSVQAEDMG |
| | QYFCQQGINNPLTFGDGTKLEIKR |
| 314 | DVVLTQTPLSLSVSFGDQVSISCRSSQSLV |
| | NSYGNTFLSWYLHKPGQSPQLLIFGISNRF |
| | SGVPDRFSGSGSGTDFTLKISTIKPEDLGM |
| | YYCLQGTHQPPTFGPGTKLEVK |
| 315 | EVQLVESGGGLTQPGKSLKLSCEASGFTFS |
| | SFTMHWVRQSPGKGLEWVAFIRSGSGIVFY |
| | ADAVRGRFTISRDNAKNLLFLQMNDLKSED |
| | TAMYYCARRPLGHNTFDSWGQGTLVTVSS |
| 316 | EVQLQQSGPELVKPGTSMKISCKASGYSFT |
| | GHLMNWVKQSHGKNLEWIGLIIPYNGGTSY |
| | NQKFKGKATLTVDKSSSTAYMELLSLTSDD |
| | SAVYFCSRGLRGFYAMDYWGQGTSVTVSS |
| 317 | KSSQSLYYSGVKENLLA |
| 318 | ASIRFT |
| 319 | QQGINNPLT |
| 320 | GFTFSSFTMH |
| 321 | FIRSGSGIVFYADAVRG |
| 322 | RPLGHNTFDS |
| 323 | RSSQSLVNSYGNTFLS |
| 324 | GISNRFS |
| 325 | LQGTHQPPT |
| 326 | GYSFTGHLMN |
| 327 | LIIPYNGGTSYNQKFKG |
| 328 | GLRGFYAMDY |
| 329 | EVQLVQSGAEVKKPGSSVKVSCKASGYTFS |
| | SYVMHWVRQAPGQGLEWIGYIDPYNDGAKY |
| | AQKFQGRVTLTSDKSTSTAYMELSSLRSED |
| | TAVYYCARGGPYGWYFDVWGQGTTVTVSS |
| 330 | DIQMTQSPSSLSASVGDRVTITCRASEHIY |
| | SYLSWYQQKPGKVPKLLIYNAKTLAEGVPS |
| | RFSGSGSGTDFTLTISSLQPEDVATYYCQH |
| | HFGSPLTFGQGTRLEIK |
| 331 | RASEHIYSYLS |
| 332 | NAKTLAE |
| 333 | QHHFGSPLT |
| 334 | SYVMH |
| 335 | YIDPYNDGAKYAQKFQG |
| 336 | GGPYGWYFDV |
| 337 | EVQLQQSGPELVKPGSSVKMSCKASGYTFS |
| | SYVMHWVKQKPGQGLEWIGYIDPYNDGAKY |
| | NEKFKGKATLTSDKSSSTAYMELSSLTSED |
| | SAVYYCARGGPYGWYFDVWGAGTTVTVSS |
| 338 | DIQMTQSPASLSASVGETVTITCRASEHIYS |
| | YLSWYQQKQGKSPQLLVYNAKTLAEGVPSR |
| | FSGSGSGTQFSLKINSLQPEDFGTYYCQHH |
| | FGSPLTFGAGTTLELK |
| 339 | EVQLQESGPGLVKPSQSLSLTCSVTGSSIA |
| | SDYWGWIRKFPGNKMEWMGFITYSGSTSYN |
| | PSLKSRISITRDTSKNQFFLQLHSVTTDDT |
| | ATYSCARMPSFITLASLSTWEGYFDFWGPG |
| | TMVTVSS |
| 340 | DIQMTQSPSLLSASVGDRVTLNCKASQSIH |
| | KNLAWYQQKLGEAPKFLIYYANSLQTGIPS |
| | RFSGSGSGTDFTLTISGLQPEDVATYFCQQ |
| | YYSGWTFGGGTKVELK |
| 341 | DVQLQESGPGLVKPSQSLSLTCTVTGYSIT |
| | SDYAWNWIRQFPGNKLEWMGYISNSGSASY |
| | NPSLKSRISITRDTSKNQFFLQLNSVTTED |
| | TATYYCATLIYYDYGGAMNFWGQGTSVTVS |
| | S |
| 342 | DIVMTQSHKFMSTSVGDRVSITCKASQGVS |
| | TTVAWYQQKPGQSPKLLIYSASYRYTGVPD |
| | RFTGSGSGTDFTFTISSVQSEDLAVYYCQH |
| | YYSTPWTFGGGTKLEIK |
| 343 | QVHLQESGPGLVKPSETLSLTCTVSGGSVS |
| | SGIYYWSWIRQPPGKGLEWIGYIYYSGSTN |
| | YNPSLKSRVTISVDTSKNQFSLKLSSVTAA |
| | DTAVYYCARDYYVSGNYYNVDYYFFGVDVW |
| | GQGTTVTVSS |
| 344 | QVQLQESGPGLVKPSETLSLTCTVSGGSVS |
| | SGIYYWSWIRQPPGKGLEWIGYIYYSGSTN |
| | YNPSLKSRVTISVDTSKNQFSLKLSSVTAA |
| | DTAVYYCARDYYVSGNYYNVDYYFFGVDVW |
| | GQGTTVTVSS |
| 345 | EIVLTQSPATLSLSPGERATLSCRASQSVS |
| | SYLAWYQQKPGQAPRLLIYDASNRATGIPA |
| | RFSGSGSGTDFTLTISSLEPEDFAVYYCQQ |
| | RSNWPPLFTFGPGTKVDIK |
| 346 | RASQSVSSYLA |
| 347 | DASNRAT |
| 348 | QQRSNWPPLFT |
| 349 | SGIYYWS |
| 350 | YIYYSGSTNYNPSLKS |
| 351 | DYYVSGNYYNVDYYFF |
| 352 | EVQLVESGGGLVQPGGSLRLSCAASGFTFS |
| | SYGMSWVRQAPGKGLEWVANIKQDGSEKYY |
| | VDSVKGRFTISRDNAKNSLYLQMNSLRAED |
| | TAVYYCARVSYYYDSSKLRWAEYFQHWGQG |
| | TLVTVSS |
| 353 | EIVLTQSPATLSLSPGERATLSCRASQSVS |
| | SYLAWYQQKPGQAPRLLIYDASKRATGIPA |
| | RFSGSGSGTDFTLTISSLEPEDFAVYYCQQ |
| | VHNFPLTFGGGTKVEIK |
| 354 | EVQLVESGGGWQPGRSLRLSCAASGFTFES |
| | YGMVWVRQAPGKGLEWVASILYDGSNRYYA |
| | DSVKGRFTVSRDNSKNTLYLQMNSLRAEDT |
| | AVYYCARVSYYYDSVELRWAEYFQHWGQGT |
| | LVTVSS |
| 355 | EIVLTQSPATLSLSPGERATLSCRASQSVS |
| | SYLAWYQQKPGQAPRLLIYDASKRATGIPA |
| | RFSGSGSGTDFTLTISSLEPEDFAVYYCQQ |
| | VHNFPLTFGGGTKVEIK |
| 356 | QVQLVQSGAEVKKPGASVKVSCKASGYTFT |
| | SYYMHWVRQAPGQGLEWMGVINPSGGSTSY |
| | AQKFQGRVTMTRDTSTSTVYMELSSLRSED |
| | TAVYYCARDHSDYWSGILDVWGQGTMVTVS |
| | S |
| 357 | EIVLTQSPGTLSLSPGERATLSCRASQSVR |
| | SSYLAWYQQKPGQAPRLLIYGASSRATGIP |
| | DRFSGSGSGTDFTLTISRLEPEDFAVYYCQ |
| | QYFSPPWTFGGGTKVEIK |
| 358 | QVQLVQSGAEVKKPGASVKVSCKASGYTFE |
| | KYYMHWVRQAPGQGLEWMGVIGPSGASTSY |
| | AQKFQGRVTMTRDTSTSTVYMELSSLRSED |
| | TAVYYCARDHSDYWSGILHSWGQGTMVTVS |
| | S |
| 359 | EIVLTQSPGTLSLSPGERATLSCRASQSVR |
| | SSYLAWYQQKPGQAPRLLIYGASSRATGIP |
| | DRFSGSGSGTDFTLTISRLEPEDFAVYYCQ |
| | QYFSPPWTFGGGTKVEIK |
| 360 | QVQLVQSGAEVKKPGASVKVSCKASGYTFT |
| | SYYMHWVRQAPGQGLEWMGVIGPSGASTSY |
| | AQKFQGRVTLTRDTSTSTVYMELSSLRSED |
| | TAVYYCARDHSDYWSGIMEVWGQGTMVTVS |
| | S |
| 361 | EIVLTQSPGTLSLSPGERATLSCRASQSVR |
| | SSYLAWYQQKPGQAPRLLIYGASSRATGIP |
| | DRFSGSGSGTDFTLTISRLEPEDFAVYYCQ |
| | QYFSPPWTFGGGTKVEIK |
| 362 | QVQLVQSGAEVKKPGASVKVSCKASGYTFT |
| | SYYMHWVRQAPGQGLEWMGVIGPSGASTSY |
| | AQKFQGRVTLTRDTSTSTVYMELSSLRSED |
| | TAVYYCARDHSDYWSGIMEVWGQGTTVTVS |
| | S |
| 363 | EIVLTQSPGTLSLSPGERATLSCRASQSVR |
| | SSYLAWYQQKPGQAPRLLIYGASSRATGIP |
| | DRFSGSGSGTDFTLTISRLEPEDFAVYYCQ |
| | QYFSPPWTFGGGTKVEIK |
| 364 | QVQLQESGPGLVKPSETLSLTCAVSGYSIS |
| | SGYYWAWIRQPPGKGLEWIGSIYHSGSTYY |
| | NPSLKSRVTISVDTSKNQFSLKLSSVTAAD |
| | TAVYYCAIEGANYYDFGYVAFDIWGQGTMV |
| | TVSS |
| 365 | EIVMTQSPATLSVSPGERATLSCRASQSVS |
| | SNLAWYQQKPGQAPRLLIYGASTRATGIPA |
| | RFSGSGSGTEFTLTISSLQSEDFAVYYCQQ |
| | SPPWPRTFGGGTKVEIK |
| 366 | QVQLQESGPGLVKPSETLSLTCTVSGGSIS |
| | SGSYYLAWIRQPPGKGLEWIGSIFRSGSTY |
| | YNPSLESRVTISVDTSKNQFSLKLSSVTAA |
| | DTAVYYCAIEGANFKDFGYVAFDIWGQGTM |
| | VTVSS |
| 367 | EIVMTQSPATLSVSPGERATLSCRASQSVS |
| | SNLAWYQQKPGQAPRLLIYGASTRATGIPA |
| | RFSGSGSGTEFTLTISSLQSEDFAVYYCQQ |
| | SPPWPRTFGGGTKVEIK |
| 368 | QLQLQESGPGLVKPSETLSLTCTVSGGSIS |
| | SSRYYWAWIRQPPGKGLEWIGSIGTSGSTY |
| | YNPSLKSRVTISVDTSKNQFSLKLSSVTAT |
| | DTAVYYCAIEGANFRDFGYVAFDIWGQGTM |
| | VTVSS |
| 369 | EIVMTQSPATLSVSPGERATLSCRASQSVS |
| | SNLAWYQQKPGQAPRLLIYGASTRATGIPA |
| | RFSGSGSGTEFTLTISSLQSEDFAVYYCQQ |
| | SPPWPRTFGGGTKVEIK |
| 370 | QVQLVQSGAEVKKPGSSVKVSCKASGGTFS |
| | SYAISVWRQAPGQGLEWMGGIIPIFGTANY |
| | AQKFQGRVTITADESTSTAYMELSSLRSED |
| | TAVYYCARLHLGSSAYYGMDVWGQGTTVTV |
| | SS |
| 371 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | RYVFPPTFGGGTKVEIK |
| 372 | QVQLVQSGAEVKKPGSSVKVSCKASGGTFQ |
| | NYAISVWRQAPGQGLEWMGVIVPIFGTANY |
| | AQKFQGRVTVTADESTSTAYMELSSLRSED |
| | TAVYYCARLHLGQKAYYGMDVWGQGTTVTV |
| | SS |
| 373 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | RYVFPPTFGGGTKVEIK |
| 374 | EVQLVESGGGLVQPGRSLRLSCAASGFTFG |
| | DYAMHVWRQAPGKGLEVWSGITWNSGSIGY |
| | ADSVKGRFTISRDNAKNSLYLQMNSLRAED |
| | TALYYCAKPVPKSRGLDVWGQGTMVTVSS |
| 375 | DIQLTQSPSSVSASVGDRVTITCRASQGIS |
| | SWLAWYQQKPGKAPKLLIYGASSLQSGVPS |
| | RFSGSGSGTDFTLTISSLQPEDFATYYCQQ |
| | AFYLPWTFGGGTKVEIK |
| 376 | QVQLVESGGGLVQPGRSLRLSCAASGFTFR |
| | DYAMHVWRQAPGKGLEVWSGITWNSGLIGY |
| | ADSVKGRFTISRDNAKNSLYLQMNSLRAED |
| | TALYYCAKPVPRLRGLDVWGQGTMVTVSS |
| 377 | DIQLTQSPSSVSASVGDRVTITCRASQGIS |
| | SWLAWYQQKPGKAPKLLIYGASSLQSGVPS |
| | RFSGSGSGTDFTLTISSLQPEDFATYYCQQ |
| | AFYLPWTFGGGTKVEIK |
| 378 | EVQLVESGGGVVQPGRSLRLSCAASGFTFG |
| | SYYMHWVRQAPGKGLEVWAVIWPDGSNKLY |
| | ADSVKGRFTISRDNSKNTLYLQMNSLRAED |
| | TALYYCAKPVPKSRALDVWGQGTMVTVSS |
| 379 | DIQLTQSPSSVSASVGDRVTITCRASQGIS |
| | SWLAWYQQKPGKAPKLLIYGASSLQSGVPS |
| | RFSGSGSGTDFTLTISSLQPEDFATYYCQQ |
| | AFYLPWTFGGGTKVEIK |
| 380 | QVQLVESGGGVVQPGRSLRLSCAASGFTFS |
| | SSYMHWVRQAPGKGLEVWAVIGADGSNKYY |
| | ADSVEGRFTISRDNSKNTLYLQMNSLRAED |
| | TALYYCAKPVPRRRGLDVWGQGTMVTVSS |
| 381 | DIQLTQSPSSVSASVGDRVTITCRASQGIS |
| | SWLAWYQQKPGKAPKLLIYGASSLQSGVPS |
| | RFSGSGSGTDFTLTISSLQPEDFATYYCQQ |
| | AFYLPWTFGGGTKVEIK |
| 382 | QLQLQESGPGLVKPSETLSLTCTVSGGSIS |
| | SGSYYLAWIRQPPGKGLEWIGSIFRSGSTY |
| | YNPSLESRVTISVDTSKNQFSLKLSSVTAA |
| | DTAVYYCAIEGANFKDFGYVAFDIWGQGTT |
| | VTVSS |
| 383 | EIVMTQSPATLSVSPGERATLSCRASQSVS |
| | SNLAWYQQKPGQAPRLLIYGASTRATGIPA |
| | RFSGSGSGTEFTLTISSLQSEDFAVYYCQQ |
| | SPPWPRTFGGGTKVEIK |
| 384 | QLQLQESGPGLVKPSETLSLTCTVSGGSIS |
| | SGSYYLAWIRQPPGKGLEWIGSIFRSGSTY |
| | YNPSLESRVTISVDTSKNQFSLKLSSVTAA |
| | DTAVYYCAIEGANFKDFGYVAFDIWGQGTT |
| | VTVSS |
| 385 | EIVLTQSPATLSLSPGERATLSCRASQSVS |
| | SYLAWYQQKPGQAPRLLIYDASNRATGIPA |
| | RFSGSGSGTDFTLTISSLEPEDFAVYYCQQ |
| | ENPRPRTFGGGTKVEIK |
| 386 | QLQLQESGPGLVKPSETLSLTCTVSGGSIS |
| | SGSYYLAWIRQPPGKGLEWIGSIFRSGSTY |
| | YNPSLESRVTISVDTSKNQFSLKLSSVTAA |
| | DTAVYYCAIEGANFKDFGYVAFDIWGQGTT |
| | VTVSS |
| 387 | EIVMTQSPATLSVSPGERATLSCRASKSVS |
| | SNLAWYQQKPGQAPRLLIYFASTRATGIPA |
| | RFSGSGSGTEFTLTISSLQSEDFAVYYCQQ |
| | TSPWPRTFGGGTKVEIK |
| 388 | QVQLVQSGAEVKKPGSSVKVSCKASGGTFS |
| | SYAISWVRQAPGQGLEWMGGIIPIFGTANY |
| | AQKFQGRVTITADESTSTAYMELSSLRSED |
| | TAVYYCAREAQSYRVPFDIWGQGTMVTVSS |
| 389 | EIVMTQSPATLSVSPGERATLSCRASQSVS |
| | SNLAWYQQKPGQAPRLLIYGASTRATGIPA |
| | RFSGSGSGTEFTLTISSLQSEDFAVYYCQQ |
| | YAIWPPFTFGGGTKVEIK |
| 390 | QVQLVQSGAEVKKPGSSVKVSCKASGGTFS |
| | SYLISWVRQAPGQGLEWMGGIYPIFATANY |
| | AQKFQGRVTITADESTSTAYMELSSLRSED |
| | TAVYYCAREAQSYRVPFDIWGQGTMVTVSS |
| 391 | EIVMTQSPATLSVSPGERATLSCRASQSVS |
| | SNLAWYQQKPGQAPRLLIYGASTRATGIPA |
| | RFSGSGSGTEFTLTISSLQSEDFAVYYCQQ |
| | YAIWPPFTFGGGTKVEIK |
| 392 | QVQLQESGPGLVKPSQTLSLTCTVSGGSIE |
| | SGLYYWGWIRQPPGKGLEWIGSIYYSGSTY |
| | YNPSLKSRATISVDTSKNQFSLKLSSVTAA |
| | DTAVYYCARDGVLALNKRSFDIWGQGTMVT |
| | VSSASTKGPSVFPLAPSSKSTSGGTAALGC |
| | LVKDYFPEPVTVSWNSGALTSGVHTFPAVL |
| | QSSGLYSLSSVVTVPSSSLGTQTYICNVNH |
| | KPSNTKVDKRVEPKSCDKTHTCPPCPAPEL |
| | LGGPSVFLFPPKPKDTLMISRTPEVTCVVV |
| | DVSHEDPEVKFNWYVDGVEVHNAKTKPREE |
| | QYNSTYRVVSVLTVLHQDWLNGKEYKCKVS |
| | NKALPAPIEKTISKAKGQPREPQVYTLPPS |
| | REEMTKNQVSLTCLVKGFYPSDIAVEWESN |
| | GQPENNYKTTPPVLDSDGSFFLYSKLTVDK |
| | SRWQQGNVFSCSVMHEALHNHYTQKSLSLS |
| | PGK |
| 393 | EIVLTQSPGTLSLSPGERATLSCRASQSVS |
| | SSYLAWYQQKPGQAPRLLIYGASSRATGIP |
| | DRFSGSGSGTDFTLTISRLEPEDFAVYYCQ |
| | QHTVRPPLTFGGGTKVEIKRTVAAPSVFIF |
| | PPSDEQLKSGTASVVCLLNNFYPREAKVQW |
| | KVDNALQSGNSQESVTEQDSKDSTYSLSST |
| | LTLSKADYEKHKVYACEVTHQGLSSPVTKS |
| | FNRGEC |
| 394 | EIVLTQSPGTLSLSPGERATLSCRASQSVS |
| | SSYLAWYQQKPGQAPRLLIYGASSRATGIP |
| | DRFSGSGSGTDFTLTISRLEPEDFAVYYCQ |
| | QHTVRPPLTFGGGTKVEIK |
| 395 | QVQLQESGPGLVKPSQTLSLTCTVSGGSIE |
| | SGLYYWGWIRQPPGKGLEWIGSIYYSGSTY |
| | YNPSLKSRATISVDTSKNQFSLKLSSVTAA |
| | DTAVYYCARDGVLALNKRSFDIWGQGTMVT |
| | VSS |
| 396 | DYYMY |
| 397 | YITKGGGSTYYPDTVKG |
| 398 | QTNYDFTMDY |
| 399 | KASQDVGTSVA |
| 400 | WASARHT |
| 401 | QQYSSYPLT |
| 402 | EVKLVESGGGLVQPGGSLKLSCATSGFTFS |
| | DYYMYWIRQTPEKRLEWVAYITKGGGSTYY |
| | PDTVKGRFTISRDNAKNTLYLQVSRLKSED |
| | TAIYYCARQTNYDFTMDYWGQGTSVTVSS |
| 403 | DIVMTQSHKFMSTSVGDRVSIICKASQDVG |
| | TSVAWYQQKPGQSPNLLIYWASARHTGVPD |
| | RFTGSGSGTDFTLTISNVQSEDLADYFCQQ |
| | YSSYPLTFGAGTKLELK |
| 404 | EVQLVETGGGLIQPGRSLRLSCAASGFTFS |
| | SYAMHWVRQAPGKGLEWVAVISYAGEVKYY |
| | ADSVKGRFTISRDNSKNTLYLQMNSLRAED |
| | TAVYYCARDPLPLHYYGMDVWGQGTTVTVS |
| | S |
| 405 | QSALTQPRSASGNPGQRVTISCSGSSSNMG |
| | RRPVNWYQQIPGTAPKLLIYSQNQRPSGVP |
| | DRFSGSQSGTSASLTISGLQSEDEAEYFCA |
| | VWDDIGRVLQLGGGTQLAVLG |
| 406 | EVQLVETGGGLIQPGRSLRLSCAASGFTFS |
| | SYAMHWVRQAPGKGLEWVAVISYDGTPVYY |
| | ADSVKGRFTISRDNSKNTLYLQMNSLRAED |
| | TAVYYCARDPLPLHYYGMDVWGQGTTVTVS |
| | S |
| 407 | QSALTQPRSASGNPGQRVTISCSGSSSNMG |
| | RRPVNWYQQIPGTAPKLLIYSQNQRPSGVP |
| | DRFSGSQSGTSASLTISGLQSEDEAEYFCA |
| | VWDGDRRSLQLGGGTQLAVLG |
| 408 | EVQLVESGGGLVQPGGSLRLSCAASGFTFS |
| | SYIMSWVRQAPGKGIEWVATISGGGTNTYY |
| | ADSVKGRFTISRDNSKNTLYLQMNSLRAED |
| | TAVYYCAKWLLSYYAMDYWGQGTLVTVSS |
| 409 | DIQMTQSPSSISASVGDRITITCRASQNIN |
| | VWLSWYQQKPGKAPKLLIYKASKSHTGVPS |
| | RFSGSGSGTDFTLTISSLQPEDFATYYCQQ |
| | GQSYPYTFGQGTKLEIK |
| 410 | QVQLVQSGAEVKKPGASVKVSCKASGYTFT |
| | SYYMSWVRQAPGQGLEWMGIINPSGGSTSY |
| | AQKFQGRVTMTRDTSTSTVYMELSSLRSED |
| | TAVYYCARARYPSSWPYGMDVWGQGTTVTV |
| | SS |
| 411 | QVQLVQSGAEVKKPGASVKVSCKASGYTFL |
| | SYYMNWVRQAPGQGLEWMGIIDPSGGRTSY |
| | AQKFQGRVTMTRDTSTSTVYMELSSLRSED |
| | TAVYYCARARYPSSWPYGTDVWGQGTTVTV |
| | SS |
| 412 | QVQLVQSGAEVKKPGASVKVSCKASGYTFR |
| | SYYMSWVRQAPGQGLEWMGIIDPSGGRTSF |
| | AQKFQGRVTMTRDTSTSTVYMELSSLRSED |
| | TAVYYCARARYPESWPYGMDVWGQGTTVTV |
| | SS |
| 413 | QVQLVQSGAEVKKPGASVKVSCKASGYTFG |
| | SYYMGWVRQAPGQGLEWMGIIDPSGGRTSY |
| | ARKFQGRVTMTRDTSTSTVYMELSSLRSED |
| | TAVYYCARARTPSSWPYGMDVWGQGTTVTV |
| | SS |
| 414 | QLQLQESGPGLVKPSETLSLTCTVSGGSIS |
| | SSRYYWGWIRQPPGKGLEWIGSIYYSGSTY |
| | YNPSLKSRVTISVDTSKNQFSLKLSSVTAA |
| | DTAVYYCARDGLYHTPEYFQHWGQGTLVTV |
| | SS |
| 415 | QLQLQESGPGLVKPSETLSLTCTVSGGSIG |
| | SSQYYWGWIRQPPGKGLEWIGSIYRSGGTY |
| | YNPSLKSRVTISVDTSKNQFSLKLSSVTAA |
| | DTAVYYCARDGLYHTPEYFQHWGQGTLVTV |
| | SS |
| 416 | QLQLQESGPGLVKPSETLSLTCTVSGGSIS |
| | SSRYYWGWIRQPPGKGLEWIGSIYYSGSTY |
| | YNPSLKSRVTISVDTSKNQFSLKLSSVTAA |
| | DTAVYYCARDGLYHTPEYFQHWGQGTLVTV |
| | SS |
| 417 | EVQLVESGGGLVQPGGSLRLSCAASGFTFS |
| | SYSMNWVRQAPGKGLEWVSYISGSSSTIYY |
| | ADSVKGRFTISRDNAKNSLYLQMNSLRAED |
| | TAVYYCARHRIADSPSRAFDIWGQGTMVTV |
| | SS |
| 418 | EVQLVESGGGLVQPGGSLRLSCAASGFTFS |
| | SYSMNWVRQAPGKGLEWVSYISSSGTINYA |
| | DSVKGRFTISRDNAKNSLYLQMNSLRAEDT |
| | AVYYCARHRIADSPSRAFDIWGQGTMVTVS |
| | S |
| 419 | EVQLVESGGGLVQPGGSLRLSCAASGFTIG |
| | GYSMNWVRQAPGKGLEWVSYISSSSTIHYA |
| | DSVKGRFTISRDNAKNSLYLQMNSLRAEDT |
| | AVYYCARHRIGRSPSRAFDIWGQGTMVTVS |
| | S |
| 420 | EVQLLESGGGLVQPGGSLRLSCAASGFTFS |
| | SYAMSWVRQAPGKGLEWVSAISGSGGSTYY |
| | ADSVKGRFTISRDNSKNTLYLQMNSLRAED |
| | TAVYYCAKDPGTDSSGYYYIWRYWGQGTLV |
| | TVSS |
| 421 | EVQLLESGGGLVQPGGSLRLSCAASGFTFS |
| | PYGMSWVRQAPGKGLEWVSSISGSGGRTYY |
| | ADSVKGRFTISRDNSKNTLYLQMNSLRAED |
| | TAVYYCAKDPGTDSSGYYYIWRYWGQGTLV |
| | TVSS |
| 422 | EVQLLESGGGLVQPGGSLRLSCAASGFTFG |
| | PYGMSWVRQAPGKGLEWVSAISGSGASTWY |
| | ADSVKGRFTISRDNSKNTLYLQMNSLRAED |
| | TAVYYCAKDPGTHYSGYYYIWRYWGQGTLV |
| | TVSS |
| 423 | EVQLLESGGGLVQPGGSLRLSCAASGFTFG |
| | PYGMSWVRQAPGKGLEWVSAISGSGASTWH |
| | ADSVKGRFTISRDNSKNTLYLQMNSLRAED |
| | TAVYYCAKDPGTDSTGYYYIWRYWGQGTLV |
| | TVSS |
| 424 | QVQLVQSGAEVKKPGASVKVSCKASGYTFT |
| | SYYMHWVRQAPGQGLEWMGIINPSGGSTSY |
| | AQKFQGRVTMTRDTSTSTVYMELSSLRSED |
| | TAVYYCARDHDIAAAGRLADYWGQGTLVTV |
| | SS |
| 425 | QVQLVQSGAEVKKPGASVKVSCKASGYTFT |
| | EYYMHWVRQAPGQGLEWMGIISPSAGSTSY |
| | AQKFQGRVTMTRDTSTSTVYMELSSLRSED |
| | TAVYYCARDHDIAAAGRLADYWGQGTLVTV |
| | SS |
| 426 | QVQLVQSGAEVKKPGASVKVSCKASGYTFT |
| | EYYMHWVRQAPGQGLEWMGIISPSAGSTKY |
| | AQKFQGRVTMTRDTSTSTVYMELSSLRSED |
| | TAVYYCARDHDIRLAGRLADYWGQGTLVTV |
| | SS |
| 427 | QLQLQESGPGLVKPSETLSLTCTVSGGSIS |
| | SSSYYWGWIRQPPGKGLEWIGSIYYSGSTY |
| | YNPSLKSRVTISVDTSKNQFSLKLSSVTAA |
| | DTAVYYCAREAGRGTTGLLFDYWGQGTLVT |
| | VSS |
| 428 | QLQLQESGPGLVKPSETLSLTCTVSGGSIS |
| | SSLYYWGWIRQPPGKGLEWIGSIYYSGSTF |
| | YNPSFKSRVTISVDTSKNQFSLKLSSVTAA |
| | DTAVYYCAREAGRGTTGLLFDYWGQGTLVT |
| | VSS |
| 429 | QLQLQESGPGLVKPSETLSLTCTVSGGSIA |
| | SSVYYWGWIRQPPGKGLEWIGSIYYSGSTW |
| | YNPSLKSRVTISVDTSKNQFSLKLSSVTAA |
| | DTAVYYCAREAGRTGTGLLFDYWGQGTLVT |
| | VSS |
| 430 | DIQMTQSPSSLSASVGDRVTITCRASQSIN |
| | SYLNWYQQKPGKAPKLLIYAASSLQSGVPS |
| | RFSGSGSGTDFTLTISSLQPEDFATYYCQQ |
| | SLLTPFTFGGGTKVEIK |
| 431 | EIVLTQSPATLSLSPGERATLSCRASQSVS |
| | SYLAWYQQKPGQAPRLLIYDASNRATGIPA |
| | RFSGSGSGTDFTLTISSLEPEDFAVYYCQQ |
| | RFAFPYTFGGGTKVEIK |
| 432 | EIVLTQSPATLSLSPGERATLSCRASQSVS |
| | SYLAWYQQKPGQAPRLLIYDASNRATGIPA |
| | RFSGSGSGTDFTLTISSLEPEDFAVYYCQQ |
| | RFAHPYTFGGGTKVEIK |
| 433 | DIVMTQSPDSLAVSLGERATINCKSSQSVL |
| | FSSNNKNYLAWYQQKPGQPPKLLIYWASTR |
| | ESGVPDRFSGSGSGTDFTLTISSLQAEDVA |
| | VYYCQQSYFFPTFGGGTKVEIK |
| 434 | EIVMTQSPATLSVSPGERATLSCRASQSVS |
| | SNLAWYQQKPGQAPRLLIYSASTRATGIPA |
| | RFSGSGSGTEFTLTISSLQSEDFAVYYCQQ |
| | YVPHPPFTFGGGTKVEIK |
| 435 | DIQMTQSPSSVSASVGDRVTITCRASQGIS |
| | SWLAWYQQKPGKAPKLLISAASSLQSGVPS |
| | RFSGSGSGTDFTLTISSLQPEDFATYYCQQ |
| | AVILPITFGGGTKVEIK |
| 436 | DIQMTQSPSTLSASVGDRVTITCRASQSIS |
| | SWLAWYQQKPGKAPKLLIYKASSLESGVPS |
| | RFSGSGSGTEFTLTISSLQPDDFATYYCQQ |
| | YGILPRTFGGGTKVEIK |
| 437 | EVQLVESGGGLVQPGGSLRLSCAASGFTFS |
| | DHYMDWVRQAPGKGLEWVGRTRNKANSYTT |
| | EYAASVKGRFTISRDDSKNSLYLQMNSLKT |
| | EDTAVYYCARGQYYYGSSSRGYYYMDVWGQ |
| | GTTVTVSS |
| 438 | EIVLTQSPGTLSLSPGERATLSCRASQSVS |
| | SSYLAWYQQKPGQAPRLLIYGASSRATGIP |
| | DRFSGSGSGTDFTLTISRLEPEDFAVYYCQ |
| | QAVPSPLTFGGGTKVEIK |
| 439 | EVQLLESGGGLVQPGGSLRLSCAASGFTFS |
| | TYAMSWVRQAPGKGLEWVSAISGSGGSTYY |
| | ADSVKGRFTISRDNSKNTLYLQMNSLRAED |
| | TAVYYCAKGPRYQDRAGMDVWGQGTTVTVS |
| | S |
| 440 | DIQMTQSPSSLSASVGDRVTITCRASQSIS |
| | SYLNWYQQKPGKAPKLLIYAASSLQSGVPS |
| | RFSGSGSGTDFTLTISSLQPEDFATYYCQQ |
| | SLATPYTFGGGTKVEIK |
| 441 | QVQLVQSGAEVKKPGSSVKVSCKASGGTFS |
| | SYAISWVRQAPGQGLEWMGSIIPIFGTANY |
| | AQKFQGRVTITADESTSTAYMELSSLRSED |
| | TAVYYCARGPSEVGAILGYVWFDPWGQGTL |
| | VTVSS |
| 442 | DIVMTQSPLSLPVTPGEPASISCRSSQSLL |
| | HSNGYNYLDWYLQKPGQSPQLLIYLGSNRA |
| | SGVPDRFSGSGSGTDFTLKISRVEAEDVGV |
| | YYCMQARRIPITFGGGTKVEIK |
| 443 | QVQLVQSGAEVKKPGSSVKVSCKASGGTFS |
| | SYAISWVRQAPGQGLEWMGGIIPIFGTASY |
| | AQKFQGRVTITADESTSTAYMELSSLRSED |
| | TAVYYCARQSTWHKLYGTDVWGQGTTVTVS |
| | S |
| 444 | DIQMTQSPSSVSASVGDRVTITCRASQGIS |
| | SWLAWYQQKPGKAPKLLIYAASSLQSGVPS |
| | RFSGSGSGTDFTLTISSLQPEDFATYYCQQ |
| | GDSLPPTFGGGTKVEIK |
| 445 | QVQLVQSGAEVKKPGSSVKVSCKASGGTFL |
| | SSAISWVRQAPGQGLEWMGSLIPYFGTANY |
| | AQKFQGRVTITADESTSTAYMELSSLRSED |
| | TAVYYCARGPSEVGAILGYVWFDPWGQGTL |
| | VTVSS |
| 446 | QVQLVQSGAEVKKPGSSVKVSCKASGGTFS |
| | AWAISWVRQAPGQGLEWMGSIIPYFGKANY |
| | AQKFQGRVTITADESTSTAYMELSSLRSED |
| | TAVYYCARGPSEVSGILGYVWFDPWGQGTL |
| | VTVSS |
| 447 | QVQLVQSGAEVKKPGSSVKVSCKASGGTFL |
| | SSAISWVRQAPGQGLEWMGSIIPLFGKANY |
| | AQKFQGRVTITADESTSTAYMELSSLRSED |
| | TAVYYCARGPSEVKGILGYVWFDPWGQGTL |
| | VTVSS |
| 448 | QVQLVQSGAEVKKPGSSVKVSCKASGGTFL |
| | SSAISWVRQAPGQGLEWMGSIIPYFGKANY |
| | AQKFQGRVTITADESTSTAYMELSSLRSED |
| | TAVYYCARGPSEVKGILGYVWFDPWGQGTL |
| | VTVSS |

1. A method for the treatment of a cancer in a human in need thereof, the method comprising administering to the human a CD96 binding protein, a PD-1 antagonist, and a TIGIT antagonist, wherein the CD96 binding protein comprises CDRH1 of SEQ ID NO: 115; CDRH2 of SEQ ID NO: 145; CDRH3 of SEQ ID NO: 147; CDRL1 of SEQ ID NO: 97; CDRL2 of SEQ ID NO: 99; and CDRL3 of SEQ ID NO: 101.

2. The method of claim 1, wherein the CD96 binding protein comprises a VH region of SEQ ID NO: 86 and a VL region of SEQ ID NO: 85.

3. The method of claim 1, wherein the CD96 binding protein is an antibody.

4. The method of claim 3, wherein the CD96 antibody further comprises a human wild-type IgG1 Fc domain.

5. The method of claim 4, wherein the CD96 antibody comprises a heavy chain of SEQ ID NO: 165 and a light chain of SEQ ID NO: 166.

6. The method of claim 1, wherein the TIGIT antagonist is an anti-TIGIT antibody.

7. The method of claim 6, wherein the anti-TIGIT antibody comprises a VH region of SEQ ID NO: 362 and a VL region of SEQ ID NO: 363.

8. The method of claim 7, wherein the anti-TIGIT antibody is EOS884488.

9. The method of claim 1, wherein the PD-1 antagonist is an anti-PD-1 antibody.

10. The method of claim 9, wherein the anti-PD-1 antibody comprises a heavy chain sequence of SEQ ID NO: 160 and a light chain sequence of SEQ ID NO: 161.

11. The method of claim 10, wherein the anti-PD-1 antibody is dostarlimab.

12. The method of claim 1, wherein the PD-1 antagonist and TIGIT antagonist are administered to the human in a ratio of about 1.0:0.80 to about 1:0:1.2 (PD-1 antagonist:TIGIT antagonist).

13. The method of claim 1, wherein the CD96 binding protein, PD-1 antagonist, and the TIGIT antagonist are administered to the human simultaneously or sequentially in any order.

14. The method of claim 1, wherein the human is administered a pharmaceutical composition comprising the CD96 binding protein, the PD-1 antagonist, and the TIGIT antagonist.

15. A combination comprising a CD96 binding protein, a PD-1 antagonist, and a TIGIT antagonist for use in the treatment of a cancer, wherein the CD96 binding protein comprises CDRH1 of SEQ ID NO: 115; CDRH2 of SEQ ID NO: 145; CDRH3 of SEQ ID NO: 147; CDRL1 of SEQ ID NO: 97; CDRL2 of SEQ ID NO: 99; and CDRL3 of SEQ ID NO: 101.

16. (canceled)

17. The method of claim 1, wherein the cancer is a solid tumor.

18. The method of claim 1, wherein the cancer is selected from the group consisting of ovarian cancer, lung cancer, gastric cancer, bladder cancer, colorectal cancer, liver cancer, renal cancer, and head and neck squamous cell carcinoma.

19. The method of claim 1, wherein the cancer is lung cancer.

20. The method of claim 19, wherein the lung cancer is non-small cell lung cancer (NSCLC).


SEQUENCE LISTING (448 sequences)

SEQ ID NO: 1 - protein, 106 aa, Artificial Sequence
  source: /note=“Description of Artificial Sequence Synthetic polypeptide”
DIQLTQSPSSLSASVGDRVTITCRASQSISSYLNWYQQKPGKAPKLLIYAASSLQSGVPSRFSGSGSGTDFTLTISSLQPEDFATYYCQQVLHTITFGGGTKVEIK

SEQ ID NO: 2 - protein, 122 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARGAGYYGDKDPMDVWGQGTTVTVSS

SEQ ID NO: 3 - DNA, 318 bp, Artificial Sequence
  source: /note=“Description of Artificial Sequence Synthetic polynucleotide”
gacatccagctgacccagagccctagcagcctgagcgccagcgtgggagacagggtgaccatcacctgcagggccagccagtccatcagcagctacctgaactggtaccagcagaagcccggcaaggcccccaagctgctgatctacgccgcaagctcactgcagagcggcgtgccctctaggtttagcggcagcggcagcggcaccgacttcaccctgaccatcagcagcctccagcccgaggacttcgccacctactactgccagcaggtgctgcacaccatcactttcggcggcggcaccaaggtggagattaag

SEQ ID NO: 4 - DNA, 366 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcagctacgccatcagctgggtgaggcaggctcccggacagggcctggagtggatgggcggcatcatccccattttcggcaccgccagctacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagc

SEQ ID NO: 5 - protein, 107 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQPYFSPPTFGGGTKVEIK

SEQ ID NO: 6 - protein, 118 aa, Artificial Sequence
  source: /note=“Description of Artificial Sequence Synthetic polypeptide”
QVQLVQSGAEVKKPGSSVKVSCKASGGTFSYNAISWVRQAPGQGLEWMGGIIPIMGTARYAQKFQGRVTITADESTSTAYMELSSLRSEDTAVYYCARLLGESGMDVWGQGTTVTVSS

SEQ ID NO: 7 - DNA, 321 bp, Artificial Sequence
  source: /note=“Description of Artificial Sequence Synthetic polynucleotide”
gacatccagatgacccagagccctagcagcctgagcgccagcgtgggagacagggtgaccatcacctgcagggccagccagtccatcagcagctacctgaactggtaccagcagaagcccggcaaggcccccaagctgctgatctacgccgcaagctcactgcagagcggcgtgccctctaggtttagcggcagcggcagcggcaccgacttcaccctgaccatcagcagcctccagcccgaggacttcgccacctactactgccagcagccctacttcagcccccccactttcggcggcggcaccaaggtggagattaag

SEQ ID NO: 8 - DNA, 354 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagctacaacgccatcagctgggtgaggcaggctcccggacagggcctggagtggatgggcggcatcatccccattatgggcaccgccaggtacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagc

SEQ ID NO: 9 - protein, 107 aa, Artificial Sequence; identical to SEQ ID NO: 5
  source: /note=“Description of Artificial Sequence Synthetic polypeptide”

SEQ ID NO: 10 - protein, 118 aa, Artificial Sequence
  source: /note=“Description of Artificial Sequence Synthetic polypeptide”
QVQLVQSGAEVKKPGSSVKVSCKASGGTFESEAISWVRQAPGQGLEWMGGIIPIFGRARYAQKFQGRVTITADESTSTAYMELSSLRSEDTAVYYCARLLGESGMDVWGQGTTVTVSS

SEQ ID NO: 11 - DNA, 321 bp, Artificial Sequence; identical to SEQ ID NO: 7
  source: /note=“Description of Artificial Sequence Synthetic polynucleotide”

SEQ ID NO: 12 - DNA, 354 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcgagagcgaggccatcagctgggtgaggcaggctcccggacagggcctggagtggatgggcggcatcatccccattttcggcagggccaggtacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagc

SEQ ID NO: 13 - protein, 107 aa, Artificial Sequence; identical to SEQ ID NO: 5
  source: /note=“Description of Artificial Sequence Synthetic polypeptide”

SEQ ID NO: 14 - protein, 118 aa, Artificial Sequence
  source: /note=“Description of Artificial Sequence Synthetic polypeptide”
QVQLVQSGAEVKKPGSSVKVSCKASGGTFSSHAISWVRQAPGQGLEWMGGIIPIFGRGKYAQKFQGRVTITADESTSTAYMELSSLRSEDTAVYYCARLLGESGMDVWGQGTTVTVSS

SEQ ID NO: 15 - DNA, 321 bp, Artificial Sequence; identical to SEQ ID NO: 7
  source: /note=“Description of Artificial Sequence Synthetic polynucleotide”

SEQ ID NO: 16 - DNA, 354 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcagccacgccatcagctgggtgaggcaggctcccggacagggcctggagtggatgggcggcatcatccccattttcggcaggggcaagtacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagc

SEQ ID NO: 17 - protein, 107 aa, Artificial Sequence; identical to SEQ ID NO: 5
  source: /note=“Description of Artificial Sequence Synthetic polypeptide”

SEQ ID NO: 18 - protein, 118 aa, Artificial Sequence
  source: /note=“Description of Artificial Sequence Synthetic polypeptide”
QVQLVQSGAEVKKPGSSVKVSCKASGGTFSGHAISWVRQAPGQGLEWMGGIIPIFGRARYAQKFQGRVTITADESTSTAYMELSSLRSEDTAVYYCARLLGESGMDVWGQGTTVTVSS

SEQ ID NO: 19 - DNA, 321 bp, Artificial Sequence; identical to SEQ ID NO: 7
  source: /note=“Description of Artificial Sequence Synthetic polynucleotide”

SEQ ID NO: 20 - DNA, 354 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcggccacgccatcagctgggtgaggcaggctcccggacagggcctggagtggatgggcggcatcatccccattttcggcagggccaggtacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagc

SEQ ID NO: 21 - protein, 107 aa, Artificial Sequence; identical to SEQ ID NO: 5
  source: /note=“Description of Artificial Sequence Synthetic polypeptide”

SEQ ID NO: 22 - protein, 118 aa, Artificial Sequence
  source: /note=“Description of Artificial Sequence Synthetic polypeptide”
QVQLVQSGAEVKKPGSSVKVSCKASGGTFSSRAISWVRQAPGQGLEWMGGIIPIMGTARYAQKFQGRVTITADESTSTAYMELSSLRSEDTAVYYCARLLGESGMDVWGQGTTVTVSS

SEQ ID NO: 23 - DNA, 321 bp, Artificial Sequence; identical to SEQ ID NO: 7
  source: /note=“Description of Artificial Sequence Synthetic polynucleotide”

SEQ ID NO: 24 - DNA, 354 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcagcagggccatcagctgggtgaggcaggctcccggacagggcctggagtggatgggcggcatcatccccattatgggcaccgccaggtacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagc

SEQ ID NO: 25 - protein, 107 aa, Artificial Sequence; identical to SEQ ID NO: 5
  source: /note=“Description of Artificial Sequence Synthetic polypeptide”

SEQ ID NO: 26 - protein, 118 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGILPIFGRANYAQKFQGRVTITADESTSTAYMELSSLRSEDTAVYYCARLLGESGMDVWGQGTTVTVSS

SEQ ID NO: 27 - DNA, 321 bp, Artificial Sequence; identical to SEQ ID NO: 7
  source: /note=“Description of Artificial Sequence Synthetic polynucleotide”

SEQ ID NO: 28 - DNA, 354 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcagctacgccatcagctgggtgaggcaggctcccggacagggcctggagtggatgggcggcatcctgcccattttcggcagggccaactacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagc

SEQ ID NO: 29 - protein, 107 aa, Artificial Sequence; identical to SEQ ID NO: 5
  source: /note=“Description of Artificial Sequence Synthetic polypeptide”

SEQ ID NO: 30 - protein, 118 aa, Artificial Sequence
  source: /note=“Description of Artificial Sequence Synthetic polypeptide”
QVQLVQSGAEVKKPGSSVKVSCKASGGTFSSSAISWVRQAPGQGLEWMGGIIPIFGRANYAQKFQGRVTITADESTSTAYMELSSLRSEDTAVYYCARLLGESGMDVWGQGTTVTVSS

SEQ ID NO: 31 - DNA, 321 bp, Artificial Sequence; identical to SEQ ID NO: 7
  source: /note=“Description of Artificial Sequence Synthetic polynucleotide”

SEQ ID NO: 32 - DNA, 354 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcagcagcgccatcagctgggtgaggcaggctcccggacagggcctggagtggatgggcggcatcatccccattttcggcagggccaactacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagc

SEQ ID NO: 33 - protein, 106 aa, Artificial Sequence; identical to SEQ ID NO: 1
  source: /note=“Description of Artificial Sequence Synthetic polypeptide”

SEQ ID NO: 34 - protein, 122 aa, Artificial Sequence
  source: /note=“Description of Artificial Sequence Synthetic polypeptide”
QVQLVQSGAEVKKPGSSVKVSCKASGGTFVNYAISWVRQAPGQGLEWMGGIIPALGTANYAQKFQGRVTITADESTSTAYMELSSLRSEDTAVYYCARGAGYYGDKDPMDVWGQGTTVTVSS

SEQ ID NO: 35 - DNA, 318 bp, Artificial Sequence; identical to SEQ ID NO: 3
  source: /note=“Description of Artificial Sequence Synthetic polynucleotide”

SEQ ID NO: 36 - DNA, 366 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcgtgaactacgccatcagctgggtgaggcaggctcccggacagggcctggagtggatgggcggcatcatccccgccctgggcaccgccaactacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagc

SEQ ID NO: 37 - protein, 106 aa, Artificial Sequence; identical to SEQ ID NO: 1
  source: /note=“Description of Artificial Sequence Synthetic polypeptide”

SEQ ID NO: 38 - protein, 122 aa, Artificial Sequence
  source: /note=“Description of Artificial Sequence Synthetic polypeptide”
QVQLVQSGAEVKKPGSSVKVSCKASGGTFSEYAIHWVRQAPGQGLEWMGNIIPIFGTAGYAQKFQGRVTITADESTSTAYMELSSLRSEDTAVYYCARGAGYYGDKDPMDVWGQGTTVTVSS

SEQ ID NO: 39 - DNA, 318 bp, Artificial Sequence; identical to SEQ ID NO: 3
  source: /note=“Description of Artificial Sequence Synthetic polynucleotide”

SEQ ID NO: 40 - DNA, 366 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcgagtacgccatccactgggtgaggcaggctcccggacagggcctggagtggatgggcaacatcatccccattttcggcaccgccggctacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagc

SEQ ID NO: 41 - protein, 106 aa, Artificial Sequence; identical to SEQ ID NO: 1
  source: /note=“Description of Artificial Sequence Synthetic polypeptide”

SEQ ID NO: 42 - protein, 122 aa, Artificial Sequence
  source: /note=“Description of Artificial Sequence Synthetic polypeptide”
QVQLVQSGAEVKKPGSSVKVSCKASGGTFWLYAISWVRQAPGQGLEWMGGIIPQLGTANYAQKFQGRVTITADESTSTAYMELSSLRSEDTAVYYCARGAGYYGDKDPMDVWGQGTTVTVSS

SEQ ID NO: 43 - DNA, 318 bp, Artificial Sequence; identical to SEQ ID NO: 3
  source: /note=“Description of Artificial Sequence Synthetic polynucleotide”

SEQ ID NO: 44 - DNA, 366 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttctggctgtacgccatcagctgggtgaggcaggctcccggacagggcctggagtggatgggcggcatcatcccccagctgggcaccgccaactacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagc

SEQ ID NO: 45 - protein, 106 aa, Artificial Sequence; identical to SEQ ID NO: 1
  source: /note=“Description of Artificial Sequence Synthetic polypeptide”

SEQ ID NO: 46 - protein, 122 aa, Artificial Sequence
  source: /note=“Description of Artificial Sequence Synthetic polypeptide”
QVQLVQSGAEVKKPGSSVKVSCKASGGTFREYAISWVRQAPGQGLEWMGGIIPVFGTANYAQKFQGRVTITADESTSTAYMELSSLRSEDTAVYYCARGAGYYGDKDPMDVWGQGTTVTVSS

SEQ ID NO: 47 - DNA, 318 bp, Artificial Sequence; identical to SEQ ID NO: 3
  source: /note=“Description of Artificial Sequence Synthetic polynucleotide”

SEQ ID NO: 48 - DNA, 366 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagggagtacgccatcagctgggtgaggcaggctcccggacagggcctggagtggatgggcggcatcatccccgtgttcggcaccgccaactacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagc

SEQ ID NO: 49 - protein, 106 aa, Artificial Sequence; identical to SEQ ID NO: 1
  source: /note=“Description of Artificial Sequence Synthetic polypeptide”

SEQ ID NO: 50 - protein, 122 aa, Artificial Sequence
  source: /note=“Description of Artificial Sequence Synthetic polypeptide”
QVQLVQSGAEVKKPGASVKVSCKASGYTFDSYAMHWVRQAPGQGLEWMGGIIPIFGTAWYAQKFQGRVTITADESTSTAYMELSSLRSEDTAVYYCARGAGYYGDKDPMDVWGQGTTVTVSS

SEQ ID NO: 51 - DNA, 318 bp, Artificial Sequence; identical to SEQ ID NO: 3
  source: /note=“Description of Artificial Sequence Synthetic polynucleotide”

SEQ ID NO: 52 - DNA, 366 bp, Artificial Sequence
  source: /note=“Description of Artificial Sequence Synthetic polynucleotide”
caggtgcagctggtgcagagcggcgccgaggtgaaaaagcccggcgccagcgtgaaggtgagctgcaaggcctccggctacaccttcgacagctacgccatgcactgggtgaggcaggctcccggacagggcctggagtggatgggcggcatcatccccattttcggcaccgcctggtacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagc

SEQ ID NO: 53 - protein, 106 aa, Artificial Sequence; identical to SEQ ID NO: 1
  source: /note=“Description of Artificial Sequence Synthetic polypeptide”

SEQ ID NO: 54 - protein, 122 aa, Artificial Sequence
  source: /note=“Description of Artificial Sequence Synthetic polypeptide”
QVQLVQSGAEVKKPGSSVKVSCKASGGTFSQYAIHWVRQAPGQGLEWMGVIIPIFGKANYAQKFQGRVTITADESTSTAYMELSSLRSEDTAVYYCARGAGYYGDKDPMDVWGQGTTVTVSS

SEQ ID NO: 55 - DNA, 318 bp, Artificial Sequence; identical to SEQ ID NO: 3
  source: /note=“Description of Artificial Sequence Synthetic polynucleotide”

SEQ ID NO: 56 - DNA, 366 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagccagtacgccatccactgggtgaggcaggctcccggacagggcctggagtggatgggcgtgatcatccccattttcggcaaggccaactacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagc

SEQ ID NO: 57 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASKRATGIPARFSGSGSGTDFTLTISSLEPEDFAVYYCQQLDNWPITFGGGTKVEIK

SEQ ID NO: 58 - protein, 126 aa, Artificial Sequence
  source: /note=“Description of Artificial Sequence Synthetic polypeptide”
QVQLQQWGAGLLKPSETLSLTCAVYGGSFRRYYWSWIRQPPGKGLEWIGEIDGWGSTNYNPSLKSRVTISVDTSKNQFSLKLSSVTAADTAVYYCARGGSVDFWSGSDYYYYMDVWGKGATVTVSS

SEQ ID NO: 59 - DNA, 321 bp, Artificial Sequence
  source: /note=“Description of Artificial Sequence Synthetic polynucleotide”
gagatcgtgctgacccagagccccgcaaccctgtccctgagccccggcgaaagggccactctgagctgcagggccagccagagcgtgagcagctacctcgcctggtaccagcagaagcccggccaggcccctaggctgctgatctacgacgccagcaagagggccaccggcattcccgccaggttcagcggcagcggcagcggcaccgacttcaccctgaccatcagcagcctggagcccgaggacttcgccgtctactactgccagcagctggacaactggcccatcaccttcgggggcggcaccaaggtggagatcaag

SEQ ID NO: 60 - DNA, 378 bp, Artificial Sequence
  source: /note=“Description of Artificial Sequence Synthetic polynucleotide”
caggtgcagctgcagcagtggggcgccggactgctgaagcccagcgagaccctgagcctgacctgcgccgtgtacggcgggtccttcaggaggtactactggagctggatcaggcagccccccggcaaaggcctggagtggatcggcgagatcgacggctggggcagcaccaactacaaccccagcctcaagagcagggtgaccatcagcgtggacaccagcaagaaccagttcagcctgaagctgagcagcgtgaccgccgccgacaccgccgtgtactattgcgccaggggcggcagcgtggacttctggagcggcagcgactactactactacatggacgtgtggggcaagggcgccaccgtcaccgtgagcagc

SEQ ID NO: 61 - protein, 107 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSLYPPRTFGGGTKVEIK

SEQ ID NO: 62 - protein, 123 aa, Artificial Sequence
  source: /note=“Description of Artificial Sequence Synthetic polypeptide”
QVQLVQSGAEVKKPGASVKVSCKASGYTFTSYPMHWVRQAPGQGLEWMGIINPSGGFTSYAQKFQGRVTMTRDTSTSTVYMELSSLRSEDTAVYYCARETAYYTTKGNWFDPWGQGTLVTVSA

SEQ ID NO: 63 - DNA, 321 bp, Artificial Sequence
  source: /note=“Description of Artificial Sequence Synthetic polynucleotide”
gacatccagatgacccagagccctagcagcctgagcgccagcgtgggagacagggtgaccatcacctgcagggccagccagtccatcagcagctacctgaactggtaccagcagaagcccggcaaggcccccaagctgctgatctacgccgcaagctcactgcagagcggcgtgccctctaggtttagcggcagcggcagcggcaccgacttcaccctgaccatcagcagcctccagcccgaggacttcgccacctactactgccagcagagcctgtacccccccaggactttcggcggcggcaccaaggtggagattaag

SEQ ID NO: 64 - DNA, 369 bp, Artificial Sequence
  source: /note=“Description of Artificial Sequence Synthetic polynucleotide”
caggtgcagctggtgcagagcggcgccgaagtgaaaaagcccggcgccagcgtgaaggtcagctgcaaggcctccgggtacaccttcaccagctaccccatgcactgggtgaggcaggcccccggccagggcctcgagtggatgggcatcatcaaccccagcggaggcttcaccagctacgcccagaagttccagggcagggtgaccatgacaagggacaccagcaccagcaccgtgtacatggagctgagcagcctgaggagcgaggacaccgccgtgtattactgcgcaagggagaccgcctactacaccaccaagggcaactggttcgacccctggggccagggcaccctggtgaccgtgagcgcc

SEQ ID NO: 65 - protein, 107 aa, Artificial Sequence; identical to SEQ ID NO: 61
  source: /note=“Description of Artificial Sequence Synthetic polypeptide”

SEQ ID NO: 66 - protein, 123 aa, Artificial Sequence
  source: /note=“Description of Artificial Sequence Synthetic polypeptide”
QVQLVQSGAEVKKPGASVKVSCKASGYTFTRAAMHWVRQAPGQGLEWMGIINPAGGYTSYAQKFQGRVTMTRDTSTSTVYMELSSLRSEDTAVYYCARETAYYTTKGNWFDPWGQGTLVTVSS

SEQ ID NO: 67 - DNA, 321 bp, Artificial Sequence; identical to SEQ ID NO: 63
  source: /note=“Description of Artificial Sequence Synthetic polynucleotide”

SEQ ID NO: 68 - DNA, 369 bp, Artificial Sequence
  source: /note=“Description of Artificial Sequence Synthetic polynucleotide”
caggtgcagctggtgcagagcggcgccgaagtgaaaaagcccggcgccagcgtgaaggtcagctgcaaggcctccgggtacaccttcaccagggccgccatgcactgggtgaggcaggcccccggccagggcctcgagtggatgggcatcatcaaccccgccggaggctacaccagctacgcccagaagttccagggcagggtgaccatgacaagggacaccagcaccagcaccgtgtacatggagctgagcagcctgaggagcgaggacaccgccgtgtattactgcgcaagggagaccgcctactacaccaccaagggcaactggttcgacccctggggccagggcaccctggtgaccgtgagctct

SEQ ID NO: 69 - protein, 107 aa, Artificial Sequence; identical to SEQ ID NO: 61
  source: /note=“Description of Artificial Sequence Synthetic polypeptide”

SEQ ID NO: 70 - protein, 123 aa, Artificial Sequence
  source: /note=“Description of Artificial Sequence Synthetic polypeptide”
QVQLVQSGAEVKKPGASVKVSCKASGYTFTTYRMHWVRQAPGQGLEWMGIINPSGGLTQYAQKFQGRVTMTRDTSTSTVYMELSSLRSEDTAVYYCARETAYYTTKGNWFDPWGQGTLVTVSS

SEQ ID NO: 71 - DNA, 321 bp, Artificial Sequence; identical to SEQ ID NO: 63
  source: /note=“Description of Artificial Sequence Synthetic polynucleotide”

SEQ ID NO: 72 - DNA, 369 bp, Artificial Sequence
  source: /note=“Description of Artificial Sequence Synthetic polynucleotide”
caggtgcagctggtgcagagcggcgccgaagtgaaaaagcccggcgccagcgtgaaggtcagctgcaaggcctccgggtacaccttcaccacctacaggatgcactgggtgaggcaggcccccggccagggcctcgagtggatgggcatcatcaaccccagcggaggcctgacccagtacgcccagaagttccagggcagggtgaccatgacaagggacaccagcaccagcaccgtgtacatggagctgagcagcctgaggagcgaggacaccgccgtgtattactgcgcaagggagaccgcctactacaccaccaagggcaactggttcgacccctggggccagggcaccctggtgaccgtgagctct

SEQ ID NO: 73 - protein, 107 aa, Artificial Sequence; identical to SEQ ID NO: 61
  source: /note=“Description of Artificial Sequence Synthetic polypeptide”

SEQ ID NO: 74 - protein, 123 aa, Artificial Sequence
  source: /note=“Description of Artificial Sequence Synthetic polypeptide”
QVQLVQSGAEVKKPGASVKVSCKASGYTFTAYQMHWVRQAPGQGLEWMGIINPAGGWTSYAQKFQGRVTMTRDTSTSTVYMELSSLRSEDTAVYYCARETAYYTTKGNWFDPWGQGTLVTVSS

SEQ ID NO: 75 - DNA, 321 bp, Artificial Sequence; identical to SEQ ID NO: 63
  source: /note=“Description of Artificial Sequence Synthetic polynucleotide”

SEQ ID NO: 76 - DNA, 369 bp, Artificial Sequence
  source: /note=“Description of Artificial Sequence Synthetic polynucleotide”
caggtgcagctggtgcagagcggcgccgaagtgaaaaagcccggcgccagcgtgaaggtcagctgcaaggcctccgggtacaccttcaccgcctaccagatgcactgggtgaggcaggcccccggccagggcctcgagtggatgggcatcatcaaccccgccggaggctggaccagctacgcccagaagttccagggcagggtgaccatgacaagggacaccagcaccagcaccgtgtacatggagctgagcagcctgaggagcgaggacaccgccgtgtattactgcgcaagggagaccgcctactacaccaccaagggcaactggttcgacccctggggccagggcaccctggtgaccgtgagctct

SEQ ID NO: 77 - protein, 107 aa, Artificial Sequence; identical to SEQ ID NO: 61
  source: /note=“Description of Artificial Sequence Synthetic polypeptide”

SEQ ID NO: 78 - protein, 123 aa, Artificial Sequence
  source: /note=“Description of Artificial Sequence Synthetic polypeptide”
QVQLVQSGAEVKKPGASVKVSCKASGYTFTRYNMHWVRQAPGQGLEWMGWINPAGGSTSYAQKFQGRVTMTRDTSTSTVYMELSSLRSEDTAVYYCARETAYYTTKGNWFDPWGQGTLVTVSA

SEQ ID NO: 79 - DNA, 321 bp, Artificial Sequence; identical to SEQ ID NO: 63
  source: /note=“Description of Artificial Sequence Synthetic polynucleotide”

SEQ ID NO: 80 - DNA, 369 bp, Artificial Sequence
  source: /note=“Description of Artificial Sequence Synthetic polynucleotide”
caggtgcagctggtgcagagcggcgccgaagtgaaaaagcccggcgccagcgtgaaggtcagctgcaaggcctccgggtacaccttcaccaggtacaacatgcactgggtgaggcaggcccccggccagggcctcgagtggatgggctggatcaaccccgccggaggcagcaccagctacgcccagaagttccagggcagggtgaccatgacaagggacaccagcaccagcaccgtgtacatggagctgagcagcctgaggagcgaggacaccgccgtgtattactgcgcaagggagaccgcctactacaccaccaagggcaactggttcgacccctggggccagggcaccctggtgaccgtgagcgcc

SEQ ID NO: 81 - protein, 107 aa, Artificial Sequence; identical to SEQ ID NO: 61
  source: /note=“Description of Artificial Sequence Synthetic polypeptide”

SEQ ID NO: 82 - protein, 123 aa, Artificial Sequence
  source: /note=“Description of Artificial Sequence Synthetic polypeptide”
QVQLVQSGAEVKKPGASVKVSCKASGYTFTKYRMHWVRQAPGQGLEWMGIINPQGGSTSYAQKFQGRVTMTRDTSTSTVYMELSSLRSEDTAVYYCARETAYYTTKGNWFDPWGQGTLVTVSA

SEQ ID NO: 83 - DNA, 321 bp, Artificial Sequence; identical to SEQ ID NO: 63
  source: /note=“Description of Artificial Sequence Synthetic polynucleotide”

SEQ ID NO: 84 - DNA, 369 bp, Artificial Sequence
  source: /note=“Description of Artificial Sequence Synthetic polynucleotide”
caggtgcagctggtgcagagcggcgccgaagtgaaaaagcccggcgccagcgtgaaggtcagctgcaaggcctccgggtacaccttcaccaagtacaggatgcactgggtgaggcaggcccccggccagggcctcgagtggatgggcatcatcaacccccagggaggcagcaccagctacgcccagaagttccagggcagggtgaccatgacaagggacaccagcaccagcaccgtgtacatggagctgagcagcctgaggagcgaggacaccgccgtgtattactgcgcaagggagaccgcctactacaccaccaagggcaactggttcgacccctggggccagggcaccctggtgaccgtgagcgcc

SEQ ID NO: 85 - protein, 106 aa, Artificial Sequence; identical to SEQ ID NO: 1
  source: /note=“Description of Artificial Sequence Synthetic polypeptide”

SEQ ID NO: 86 - protein, 122 aa, Artificial Sequence
  source: /note=“Description of Artificial Sequence Synthetic polypeptide”
QVQLVQSGAEVKKPGSSVKVSCKASGGTFVEYAISWVRQAPGQGLEWMGGIIPAFGTAQYAQKFQGRVTITADESTSTAYMELSSLRSEDTAVYYCARGAGYYGDKDPMDVWGQGTTVTVSS

SEQ ID NO: 87 - DNA, 318 bp, Artificial Sequence; identical to SEQ ID NO: 3
  source: /note=“Description of Artificial Sequence Synthetic polynucleotide”

SEQ ID NO: 88 - DNA, 366 bp, Artificial Sequence
  source: /note=“Description of Artificial Sequence Synthetic polynucleotide”
caggtgcagctggtgcagagcggcgcagaggtgaaaaagcccggcagcagcgtgaaggtgagctgcaaggcctccggcgggaccttcgtggagtacgccatcagctgggtgaggcaggctcccggacagggcctggagtggatgggcggcatcatccccgccttcggcaccgcccagtacgcccagaagttccagggaagggtcaccatcaccgccgacgagagcaccagcaccgcctacatggaactcagcagcctgaggagcgaggacaccgccgtgtactattgcgccaggggagccggctactacggcgacaaggaccccatggacgtgtggggccagggcaccaccgtgactgtgagcagc

SEQ ID NO: 89 - protein, 106 aa, Artificial Sequence; identical to SEQ ID NO: 1
  source: /note=“Description of Artificial Sequence Synthetic polypeptide”

SEQ ID NO: 90 - protein, 122 aa, Artificial Sequence
  source: /note=“Description of Artificial Sequence Synthetic polypeptide”
QVQLVQSGAEVKKPGSSVKVSCKASGGTFNEYAISWVRQAPGQGLEWMGGIVPVFGTAKYAQKFQGRVTITADESTSTAYMELSSLRSEDTAVYYCARGAGYYGDKDPMDVWGQGTTVTVSS

SEQ ID NO: 91 - DNA, 318 bp, Artificial Sequence; identical to SEQ ID NO: 3
  source: /note=“Description of Artificial Sequence Synthetic polynucleotide”

SEQ ID NO: 92 - DNA, 366 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcaacgagtacgccatcagctgggtgaggcaggctcccggacagggcctggagtggatgggcggcatcgtgcccgtgttcggcaccgccaagtacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagc

SEQ ID NO: 93 - protein, 107 aa, Artificial Sequence; identical to SEQ ID NO: 5
  source: /note=“Description of Artificial Sequence Synthetic polypeptide”

SEQ ID NO: 94 - protein, 118 aa, Artificial Sequence
  source: /note=“Description of Artificial Sequence Synthetic polypeptide”
QVQLVQSGAEVKKPGSSVKVSCKASGGTFSGYPISWVRQAPGQGLEWMGGIIPIMGTARYAQKFQGRVTITADESTSTAYMELSSLRSEDTAVYYCARLLGESGMDVWGQGTTVTVSS

SEQ ID NO: 95 - DNA, 321 bp, Artificial Sequence; identical to SEQ ID NO: 7
  source: /note=“Description of Artificial Sequence Synthetic polynucleotide”

SEQ ID NO: 96 - DNA, 354 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcggctaccccatcagctgggtgaggcaggctcccggacagggcctggagtggatgggcggcatcatccccattatgggcaccgccaggtacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagc

SEQ ID NO: 97 - protein, 11 aa, Artificial Sequence
  source: /note=“Description of Artificial Sequence Synthetic peptide”
RASQSISSYLN

SEQ ID NO: 98 - protein, 11 aa, Artificial Sequence
  source: /note=“Description of Artificial Sequence Synthetic peptide”
RASQSVSSYLA

SEQ ID NO: 99 - protein, 7 aa, Artificial Sequence
  source: /note=“Description of Artificial Sequence Synthetic peptide”
AASSLQS

SEQ ID NO: 100 - protein, 7 aa, Artificial Sequence
  source: /note=“Description of Artificial Sequence Synthetic peptide”
DASKRAT

SEQ ID NO: 101 - protein, 8 aa, Artificial Sequence
  source: /note=“Description of Artificial Sequence Synthetic peptide”
QQVLHTIT

SEQ ID NO: 102 - protein, 9 aa, Artificial Sequence
  source: /note=“Description of Artificial Sequence Synthetic peptide”
QQPYFSPPT

SEQ ID NO: 103 - protein, 9 aa, Artificial Sequence
  source: /note=“Description of Artificial Sequence Synthetic peptide”
QQLDNWPIT

SEQ ID NO: 104 - protein, 9 aa, Artificial Sequence
  source: /note=“Description of Artificial Sequence Synthetic peptide”
QQSLYPPRT

SEQ ID NO: 105 - protein, 5 aa, Artificial Sequence
  source: /note=“Description of Artificial Sequence Synthetic peptide”
SYAIS

SEQ ID NO: 106 - protein, 5 aa, Artificial Sequence
  source: /note=“Description of Artificial Sequence Synthetic peptide”
YNAIS

SEQ ID NO: 107 - protein, 5 aa, Artificial Sequence
  source: /note=“Description of Artificial Sequence Synthetic peptide”
SEAIS

SEQ ID NO: 108 - protein, 5 aa, Artificial Sequence
  source: /note=“Description of Artificial Sequence Synthetic peptide”
SHAIS

SEQ ID NO: 109 - protein, 5 aa, Artificial Sequence
  source: /note=“Description of Artificial Sequence Synthetic peptide”
GHAIS

SEQ ID NO: 110 - protein, 5 aa, Artificial Sequence
  source: /note=“Description of Artificial Sequence Synthetic peptide”
SRAIS

SEQ ID NO: 111 - protein, 5 aa, Artificial Sequence
  source: /note=“Description of Artificial Sequence Synthetic peptide”
SSAIS

SEQ ID NO: 112 - protein, 5 aa, Artificial Sequence
  source: /note=“Description of Artificial Sequence Synthetic peptide”
NYAIS

SEQ ID NO: 113 - protein, 5 aa, Artificial Sequence
  source: /note=“Description of Artificial Sequence Synthetic peptide”
EYAIH

SEQ ID NO: 114 - protein, 5 aa, Artificial Sequence
  source: /note=“Description of Artificial Sequence Synthetic peptide”
LYAIS

SEQ ID NO: 115 - protein, 5 aa, Artificial Sequence
  source: /note=“Description of Artificial Sequence Synthetic peptide”
EYAIS

SEQ ID NO: 116 - protein, 5 aa, Artificial Sequence
  source: /note=“Description of Artificial Sequence Synthetic peptide”
SYAMH

SEQ ID NO: 117 - protein, 5 aa, Artificial Sequence
  source: /note=“Description of Artificial Sequence Synthetic peptide”
QYAIH

SEQ ID NO: 118 - protein, 5 aa, Artificial Sequence
  source: /note=“Description of Artificial Sequence Synthetic peptide”
RYYWS

SEQ ID NO: 119 - protein, 5 aa, Artificial Sequence
  source: /note=“Description of Artificial Sequence Synthetic peptide”
SYPMH

SEQ ID NO: 120 - protein, 5 aa, Artificial Sequence
  source: /note=“Description of Artificial Sequence Synthetic peptide”
RAAMH

SEQ ID NO: 121 - protein, 5 aa, Artificial Sequence
  source: /note=“Description of Artificial Sequence Synthetic peptide”
TYRMH

SEQ ID NO: 122 - protein, 5 aa, Artificial Sequence
  source: /note=“Description of Artificial Sequence Synthetic peptide”
AYQMH

SEQ ID NO: 123 - protein, 5 aa, Artificial Sequence
  source: /note=“Description of Artificial Sequence Synthetic peptide”
RYNMH

SEQ ID NO: 124 - protein, 5 aa, Artificial Sequence
  source: /note=“Description of Artificial Sequence Synthetic peptide”
KYRMH

SEQ ID NO: 125 - protein, 5 aa, Artificial Sequence
  source: /note=“Description of Artificial Sequence Synthetic peptide”
GYPIS

SEQ ID NO: 126 - protein, 17 aa, Artificial Sequence
  source: /note=“Description of Artificial Sequence Synthetic peptide”
GIIPIFGTASYAQKFQG

SEQ ID NO: 127 - protein, 17 aa, Artificial Sequence
  source: /note=“Description of Artificial Sequence Synthetic peptide”
GIIPIMGTARYAQKFQG

SEQ ID NO: 128 - protein, 17 aa, Artificial Sequence
  source: /note=“Description of Artificial Sequence Synthetic peptide”
GIIPIFGRARYAQKFQG

SEQ ID NO: 129 - protein, 17 aa, Artificial Sequence
  source: /note=“Description of Artificial Sequence Synthetic peptide”
GIIPIFGRGKYAQKFQG

SEQ ID NO: 130 - protein, 17 aa, Artificial Sequence
  source: /note=“Description of Artificial Sequence Synthetic peptide”
GILPIFGRANYAQKFQG

SEQ ID NO: 131 - protein, 17 aa, Artificial Sequence
  source: /note=“Description of Artificial Sequence Synthetic peptide”
GIIPIFGRANYAQKFQG

SEQ ID NO: 132 - protein, 17 aa, Artificial Sequence
  source: /note=“Description of Artificial Sequence Synthetic peptide”
GIIPALGTANYAQKFQG

SEQ ID NO: 133 - protein, 17 aa, Artificial Sequence
  source: /note=“Description of Artificial Sequence Synthetic peptide”
NIIPIFGTAGYAQKFQG

SEQ ID NO: 134 - protein, 17 aa, Artificial Sequence
  source: /note=“Description of Artificial Sequence Synthetic peptide”
GIIPQLGTANYAQKFQG

SEQ ID NO: 135 - protein, 17 aa, Artificial Sequence
  source: /note=“Description of Artificial Sequence Synthetic peptide”
GIIPVFGTANYAQKFQG

SEQ ID NO: 136 - protein, 17 aa, Artificial Sequence
  source: /note=“Description of Artificial Sequence Synthetic peptide”
GIIPIFGTAWYAQKFQG

SEQ ID NO: 137 - protein, 17 aa, Artificial Sequence
  source: /note=“Description of Artificial Sequence Synthetic peptide”
VIIPIFGKANYAQKFQG

SEQ ID NO: 138 - protein, 16 aa, Artificial Sequence
  source: /note=“Description of Artificial Sequence Synthetic peptide”
EIDGWGSTNYNPSLKS

SEQ ID NO: 139 - protein, 17 aa, Artificial Sequence
  source: /note=“Description of Artificial Sequence Synthetic peptide”
IINPSGGFTSYAQKFQG

SEQ ID NO: 140 - protein, 17 aa, Artificial Sequence
  source: /note=“Description of Artificial Sequence Synthetic peptide”
IINPAGGYTSYAQKFQG

SEQ ID NO: 141 - protein, 17 aa, Artificial Sequence
  source: /note=“Description of Artificial Sequence Synthetic peptide”
IINPSGGLTQYAQKFQG

SEQ ID NO: 142 - protein, 17 aa, Artificial Sequence
  source: /note=“Description of Artificial Sequence Synthetic peptide”
IINPAGGWTSYAQKFQG

SEQ ID NO: 143 - protein, 17 aa, Artificial Sequence
  source: /note=“Description of Artificial Sequence Synthetic peptide”
WINPAGGSTSYAQKFQG

SEQ ID NO: 144 - protein, 17 aa, Artificial Sequence
  source: /note=“Description of Artificial Sequence Synthetic peptide”
IINPQGGSTSYAQKFQG

SEQ ID NO: 145 - protein, 17 aa, Artificial Sequence
  source: /note=“Description of Artificial Sequence Synthetic peptide”
GIIPAFGTAQYAQKFQG

SEQ ID NO: 146 - protein, 17 aa, Artificial Sequence
  source: /note=“Description of Artificial Sequence Synthetic peptide”
GIVPVFGTAKYAQKFQG

SEQ ID NO: 147 - protein, 13 aa, Artificial Sequence
  source: /note=“Description of Artificial Sequence Synthetic peptide”
GAGYYGDKDPMDV

SEQ ID NO: 148 - protein, 9 aa, Artificial Sequence
  source: /note=“Description of Artificial Sequence Synthetic peptide”
LLGESGMDV

SEQ ID NO: 149 - protein, 18 aa, Artificial Sequence
  source: /note=“Description of Artificial Sequence Synthetic peptide”
GGSVDFWSGSDYYYYMDV

SEQ ID NO: 150 - protein, 14 aa, Artificial Sequence
  source: /note=“Description of Artificial Sequence Synthetic peptide”
ETAYYTTKGNWFDP

SEQ ID NO: 151 - protein, 5 aa, Artificial Sequence
  source: /note=“Description of Artificial Sequence Synthetic peptide”
SYDMS

SEQ ID NO: 152 - protein, 17 aa, Artificial Sequence
  source: /note=“Description of Artificial Sequence Synthetic peptide”
TISGGGSYTYYQDSVKG

SEQ ID NO: 153 - protein, 7 aa, Artificial Sequence
  source: /note=“Description of Artificial Sequence Synthetic peptide”
PYYAMDY

SEQ ID NO: 154 - protein, 11 aa, Artificial Sequence
  source: /note=“Description of Artificial Sequence Synthetic peptide”
KASQDVGTAVA

SEQ ID NO: 155 - protein, 7 aa, Artificial Sequence
  source: /note=“Description of Artificial Sequence Synthetic peptide”
WASTLHT

SEQ ID NO: 156 - protein, 9 aa, Artificial Sequence
  source: /note=“Description of Artificial Sequence Synthetic peptide”
QHYSSYPWT

SEQ ID NO: 157 - protein, 9 aa, Artificial Sequence
  source: /note=“Description of Artificial Sequence Synthetic peptide”
QHYNSYPWT

SEQ ID NO: 158 - protein, 116 aa, Artificial Sequence
  source: /note=“Description of Artificial Sequence Synthetic polypeptide”
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 159 - protein, 107 aa, Artificial Sequence
  source: /note=“Description of Artificial Sequence Synthetic polypeptide”
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 160 - protein, 443 aa, Artificial Sequence
  source: /note=“Description of Artificial Sequence Synthetic polypeptide”
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 161 - protein, 214 aa, Artificial Sequence
  source: /note=“Description of Artificial Sequence Synthetic polypeptide”
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 443 aa, Artificial Sequence
  source: /note=“Description of Artificial Sequence Synthetic polypeptide”
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESDGQPENNYKTTPPVLDSDGSFFLYSRLTVDKSRWQEGNVFSCSVMHEALHNHYTQKSLSLSLGK

SEQ ID NO: 163 - protein, 443 aa, Artificial Sequence
  source: /note=“Description of Artificial Sequence Synthetic polypeptide”
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSLGK

SEQ ID NO: 164 - protein, 443 aa, Artificial Sequence
  source: /note=“Description of Artificial Sequence Synthetic polypeptide”
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESDGQPEDNYKTTPPVLDSDGSFFLYSRLTVDKSRWQEGNVFSCSVMHEALHNHYTQKSLSLSLGK

SEQ ID NO: 165 - protein, 452 aa, Artificial Sequence
  source: /note=“Description of Artificial Sequence Synthetic polypeptide”
QVQLVQSGAEVKKPGSSVKVSCKASGGTFVEYAISWVRQAPGQGLEWMGGIIPAFGTAQYAQKFQGRVTITADESTSTAYMELSSLRSEDTAVYYCARGAGYYGDKDPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 213 aa, Artificial Sequence
  source: /note=“Description of Artificial Sequence Synthetic polypeptide”
DIQLTQSPSSLSASVGDRVTITCRASQSISSYLNWYQQKPGKAPKLLIYAASSLQSGVPSRFSGSGSGTDFTLTISSLQPEDFATYYCQQVLHTITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - DNA, 1356 bp, Artificial Sequence
  source: /note=“Description of Artificial Sequence Synthetic polynucleotide”
caggtgcagctggtgcagagcggcgcagaggtgaaaaagcccggcagcagcgtgaaggtgagctgcaaggcctccggcgggaccttcgtggagtacgccatcagctgggtgaggcaggctcccggacagggcctggagtggatgggcggcatcatccccgccttcggcaccgcccagtacgcccagaagttccagggaagggtcaccatcaccgccgacgagagcaccagcaccgcctacatggaactcagcagcctgaggagcgaggacaccgccgtgtactattgcgccaggggagccggctactacggcgacaaggaccccatggacgtgtggggccagggcaccaccgtgactgtgagcagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 168 - DNA, 639 bp, Artificial Sequence
  source: /note=“Description of Artificial Sequence Synthetic polynucleotide”
gacatccagctgacccagagccctagcagcctgagcgccagcgtgggagacagggtgaccatcacctgcagggccagccagtccatcagcagctacctgaactggtaccagcagaagcccggcaaggcccccaagctgctgatctacgccgcaagctcactgcagagcggcgtgccctctaggtttagcggcagcggcagcggcaccgacttcaccctgaccatcagcagcctccagcccgaggacttcgccacctactactgccagcaggtgctgcacaccatcactttcggcggcggcaccaaggtggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 169 - protein, 213 aa, Artificial Sequence; identical to SEQ ID NO: 166
  source: /note=“Description of Artificial Sequence Synthetic polypeptide”

SEQ ID NO: 170 - protein, 452 aa, Artificial Sequence
  source: /note=“Description of Artificial Sequence Synthetic polypeptide”
QVQLVQSGAEVKKPGSSVKVSCKASGGTFNEYAISWVRQAPGQGLEWMGGIVPVFGTAKYAQKFQGRVTITADESTSTAYMELSSLRSEDTAVYYCARGAGYYGDKDPMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - DNA, 639 bp, Artificial Sequence; identical to SEQ ID NO: 168
  source: /note=“Description of Artificial Sequence Synthetic polynucleotide”

SEQ ID NO: 172 - DNA, 1356 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcaacgagtacgccatcagctgggtgaggcaggctcccggacagggcctggagtggatgggcggcatcgtgcccgtgttcggcaccgccaagtacgcccagaagttccagggaagggtcaccatcaccgccgacgagagcaccagcaccgcctacatggaactcagcagcctgaggagcgaggacaccgccgtgtactattgcgccaggggcgccggctactacggcgacaaggaccccatggacgtgtggggccagggcaccaccgtgactgtgagcagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 173 - protein, 214 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQPYFSPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 448 aa, Artificial Sequence
  source: /note=“Description of Artificial Sequence Synthetic polypeptide”
QVQLVQSGAEVKKPGSSVKVSCKASGGTFSGYPISWVRQAPGQGLEWMGGIIPIMGTARYAQKFQGRVTITADESTSTAYMELSSLRSEDTAVYYCARLLGESGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - DNA, 642 bp, Artificial Sequence
  source: /note=“Description of Artificial Sequence Synthetic polynucleotide”
gacatccagatgacccagagccctagcagcctgagcgccagcgtgggagacagggtgaccatcacctgcagggccagccagtccatcagcagctacctgaactggtaccagcagaagcccggcaaggcccccaagctgctgatctacgccgcaagctcactgcagagcggcgtgccctctaggtttagcggcagcggcagcggcaccgacttcaccctgaccatcagcagcctccagcccgaggacttcgccacctactactgccagcagccctacttcagcccccccactttcggcggcggcaccaaggtggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 176 - DNA, 1344 bp, Artificial Sequence
  source: /note=“Description of Artificial Sequence Synthetic polynucleotide”
caggtgcagctggtgcagagcggcgccgaggtgaaaaagcccggcagcagcgtgaaggtgagctgcaaggcctccggcgggaccttcagcggctaccccatcagctgggtgaggcaggctcccggacagggcctggagtggatgggcggcatcatccccattatgggcaccgccaggtacgcccagaagttccagggaagggtcaccatcaccgccgacgagagcaccagcaccgcctacatggaactcagcagcctgaggagcgaggacaccgccgtgtactattgcgccaggctgctgggcgagagcggcatggacgtgtggggccagggcaccaccgtgactgtgagcagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 177 - protein, 564 aa, Artificial Sequence
  source: /note=“Description of Artificial Sequence Synthetic polypeptide”
WPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNSGENLYFQGDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 179 - protein, 330 aa, Homo sapiens [sequence omitted]

SEQ ID NO: 180 - DNA, 990 bp, Homo sapiens
gccagcaccaagggccccagcgtgttccccctggcccccagcagcaagagcaccagcggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 181 - protein, 9 aa, Artificial Sequence
  source: /note=“Description of Artificial Sequence Synthetic peptide”
FTFSEYTMN

SEQ ID NO: 182 - protein, 17 aa, Artificial Sequence
  source: /note=“Description of Artificial Sequence Synthetic peptide”
AIVGSGDSTYYADSVKG

SEQ ID NO: 183 - protein, 16 aa, Artificial Sequence
  source: /note=“Description of Artificial Sequence Synthetic peptide”
AKDYSSGDWIDYGMDV

SEQ ID NO: 184 - protein, 11 aa, Artificial Sequence
  source: /note=“Description of Artificial Sequence Synthetic peptide”
QASQDISNYLN

SEQ ID NO: 185 - protein, 7 aa, Artificial Sequence
  source: /note=“Description of Artificial Sequence Synthetic peptide”
DASNLAT

SEQ ID NO: 186 - protein, 9 aa, Artificial Sequence
  source: /note=“Description of Artificial Sequence Synthetic peptide”
QQFDLLPPT

SEQ ID NO: 187 - protein, 9 aa, Artificial Sequence
  source: /note=“Description of Artificial Sequence Synthetic peptide”
FTFSDYAMI

SEQ ID NO: 188 - protein, 17 aa, Artificial Sequence
  source: /note=“Description of Artificial Sequence Synthetic peptide”
AISGGGESTYYADSVKG

SEQ ID NO: 189 - protein, 16 aa, Artificial Sequence
  source: /note=“Description of Artificial Sequence Synthetic peptide”
AKDYSSGDWIDYGMDV

SEQ ID NO: 190 - protein, 11 aa, Artificial Sequence
  source: /note=“Description of Artificial Sequence Synthetic peptide”
QASQDISNYLN

SEQ ID NO: 191 - protein, 7 aa, Artificial Sequence
  source: /note=“Description of Artificial Sequence Synthetic peptide”
DASNLAT

SEQ ID NO: 192 - protein, 9 aa, Artificial Sequence
  source: /note=“Description of Artificial Sequence Synthetic peptide”
QQFDLLPPT

SEQ ID NO: 193 - protein, 9 aa, Artificial Sequence
  source: /note=“Description of Artificial Sequence Synthetic peptide”
GTFDNYYIS

SEQ ID NO: 194 - protein, 17 aa, Artificial Sequence
  source: /note=“Description of Artificial Sequence Synthetic peptide”
GIFPIFGTANYAQKFQG

SEQ ID NO: 195 - protein, 16 aa, Artificial Sequence
  source: /note=“Description of Artificial Sequence Synthetic peptide”
AREVGHYSGSPYYMDV

SEQ ID NO: 196 - protein, 11 aa, Artificial Sequence
  source: /note=“Description of Artificial Sequence Synthetic peptide”
RASQSINSWLA

SEQ ID NO: 197 - protein, 7 aa, Artificial Sequence
  source: /note=“Description of Artificial Sequence Synthetic peptide”
DASSLES

SEQ ID NO: 198 - protein, 8 aa, Artificial Sequence
  source: /note=“Description of Artificial Sequence Synthetic peptide”
QQVGPYLT

SEQ ID NO: 199 - protein, 9 aa, Artificial Sequence
  source: /note=“Description of Artificial Sequence Synthetic peptide”
FTFGDYAMS

SEQ ID NO: 200 - protein, 19 aa, Artificial Sequence
  source: /note=“Description of Artificial Sequence Synthetic peptide”
FIGSKFYGGETEYTASVKG

SEQ ID NO: 201 - protein, 13 aa, Artificial Sequence
  source: /note=“Description of Artificial Sequence Synthetic peptide”
ARGPRRYTYGMDV

SEQ ID NO: 202 - protein, 11 aa, Artificial Sequence
  source: /note=“Description of Artificial Sequence Synthetic peptide”
RASQSISSYLN

SEQ ID NO: 203 - protein, 7 aa, Artificial Sequence
  source: /note=“Description of Artificial Sequence Synthetic peptide”
AASSLQS

SEQ ID NO: 204 - protein, 8 aa, Artificial Sequence
  source: /note=“Description of Artificial Sequence Synthetic peptide”
QQSSTPLT

SEQ ID NO: 205 - protein, 9 aa, Artificial Sequence
  source: /note=“Description of Artificial Sequence Synthetic peptide”
FTFDDYAVH

SEQ ID NO: 206 - protein, 17 aa, Artificial Sequence
  source: /note=“Description of Artificial Sequence Synthetic peptide”
GISWSSGLIGYADSVKG

SEQ ID NO: 207 - protein, 13 aa, Artificial Sequence
  source: /note=“Description of Artificial Sequence Synthetic peptide”
AKGPPTYQDYFDL

SEQ ID NO: 208 - protein, 11 aa, Artificial Sequence
  source: /note=“Description of Artificial Sequence Synthetic peptide”
RASQSVSRYLA

SEQ ID NO: 209 - protein, 7 aa, Artificial Sequence
  source: /note=“Description of Artificial Sequence Synthetic peptide”
DASNRAT

SEQ ID NO: 210 - protein, 10 aa, Artificial Sequence
  source: /note=“Description of Artificial Sequence Synthetic peptide”
QQVSFFPPIT

SEQ ID NO: 211 - protein, 9 aa, Artificial Sequence
  source: /note=“Description of Artificial Sequence Synthetic peptide”
FTFSGHLMS

SEQ ID NO: 212 - protein, 17 aa, Artificial Sequence
  source: /note=“Description of Artificial Sequence Synthetic peptide”
AISGSAGETYYADSVKG

SEQ ID NO: 213 - protein, 19 aa, Artificial Sequence
  source: /note=“Description of Artificial Sequence Synthetic peptide”
ARDAYYDDWSGWADWYFDL

SEQ ID NO: 214 - protein, 11 aa, Artificial Sequence
  source: /note=“Description of Artificial Sequence Synthetic peptide”
RASQSVSRYLA

SEQ ID NO: 215 - protein, 7 aa, Artificial Sequence
  source: /note=“Description of Artificial Sequence Synthetic peptide”
DASNRAT

SEQ ID NO: 216 - protein, 9 aa, Artificial Sequence
  source: /note=“Description of Artificial Sequence Synthetic peptide”
QQVSLLPPT

SEQ ID NO: 217 - protein, 9 aa, Artificial Sequence
  source: /note=“Description of Artificial Sequence Synthetic peptide”
FTFGDVAMS

SEQ ID NO: 218 - protein, 19 aa, Artificial Sequence
  source: /note=“Description of Artificial Sequence Synthetic peptide”
YIGSKAYGGETEYTASVKG

SEQ ID NO: 219 - protein, 17 aa, Artificial Sequence
  source: /note=“Description of Artificial Sequence Synthetic peptide”
ARAGHSYGSIASNWFDP

SEQ ID NO: 220 - protein, 11 aa, Artificial Sequence
  source: /note=“Description of Artificial Sequence Synthetic peptide”
RASQSISSYLN

SEQ ID NO: 221 - protein, 7 aa, Artificial Sequence
  source: /note=“Description of Artificial Sequence Synthetic peptide”
GASSLQS

SEQ ID NO: 222 - protein, 9 aa, Artificial Sequence
  source: /note=“Description of Artificial Sequence Synthetic peptide”
QQGFYTPWT

SEQ ID NO: 223 - protein, 9 aa, Artificial Sequence
  source: /note=“Description of Artificial Sequence Synthetic peptide”
GTFSSAAIS

SEQ ID NO: 224 - protein, 17 aa, Artificial Sequence
  source: /note=“Description of Artificial Sequence Synthetic peptide”
NIIPIVGIANYAQKFQG

SEQ ID NO: 225 - protein, 16 aa, Artificial Sequence
  source: /note=“Description of Artificial Sequence Synthetic peptide”
ARDTGRGYTRHFWFDP

SEQ ID NO: 226 - protein, 11 aa, Artificial Sequence
  source: /note=“Description of Artificial Sequence Synthetic peptide”
RASQSISSYLN

SEQ ID NO: 227 - protein, 7 aa, Artificial Sequence
  source: /note=“Description of Artificial Sequence Synthetic peptide”
AASSLQS

SEQ ID NO: 228 - protein, 8 aa, Artificial Sequence
  source: /note=“Description of Artificial Sequence Synthetic peptide”
QQSDILYT

SEQ ID NO: 229 - protein, 9 aa, Artificial Sequence
  source: /note=“Description of Artificial Sequence Synthetic peptide”
GTFSNYAIS

SEQ ID NO: 230 - protein, 17 aa, Artificial Sequence
  source: /note=“Description of Artificial Sequence Synthetic peptide”
GIIPIFGTANYAQKFQG

SEQ ID NO: 231 - protein, 18 aa, Artificial Sequence
  source: /note=“Description of Artificial Sequence Synthetic peptide”
ARGRGALALVGPYYGMDV

SEQ ID NO: 232 - protein, 16 aa, Artificial Sequence
  source: /note=“Description of Artificial Sequence Synthetic peptide”
RSSQSLLHSNGYNYLD

SEQ ID NO: 233 - protein, 7 aa, Artificial Sequence
  source: /note=“Description of Artificial Sequence Synthetic peptide”
LGSHRAS

SEQ ID NO: 234 - protein, 8 aa, Artificial Sequence
  source: /note=“Description of Artificial Sequence Synthetic peptide”
MQALRAPT

SEQ ID NO: 235 - protein, 9 aa, Artificial Sequence
  source: /note=“Description of Artificial Sequence Synthetic peptide”
FTFGDYAMS

SEQ ID NO: 236 - protein, 19 aa, Artificial Sequence
  source: /note=“Description of Artificial Sequence Synthetic peptide”
FIGSKAYGGTTEYTASVKG

SEQ ID NO: 237 - protein, 13 aa, Artificial Sequence
  source: /note=“Description of Artificial Sequence Synthetic peptide”
ARGPRRYTYGMDV

SEQ ID NO: 238 - protein, 11 aa, Artificial Sequence
  source: /note=“Description of Artificial Sequence Synthetic peptide”
RASQSISSYLN

SEQ ID NO: 239 - protein, 7 aa, Artificial Sequence
  source: /note=“Description of Artificial Sequence Synthetic peptide”
AASSLQS

SEQ ID NO: 240 - protein, 8 aa, Artificial Sequence
  source: /note=“Description of Artificial Sequence Synthetic peptide”
QQSSTPLT

SEQ ID NO: 241 - protein, 9 aa, Artificial Sequence
  source: /note=“Description of Artificial Sequence Synthetic peptide”
GTFSSYAIS

SEQ ID NO: 242 - protein, 17 aa, Artificial Sequence
  source: /note=“Description of Artificial Sequence Synthetic peptide”
GIIPISGTANYAQKFQG

SEQ ID NO: 243 - protein, 16 aa, Artificial Sequence
  source: /note=“Description of Artificial Sequence Synthetic peptide”
ARDTGRGYTRHFWFDP

SEQ ID NO: 244 - protein, 11 aa, Artificial Sequence
  source: /note=“Description of Artificial Sequence Synthetic peptide”
RASQSISSYLN

SEQ ID NO: 245 - protein, 7 aa, Artificial Sequence
  source: /note=“Description of Artificial Sequence Synthetic peptide”
AASSLQS

SEQ ID NO: 246 - protein, 8 aa, Artificial Sequence
  source: /note=“Description of Artificial Sequence Synthetic peptide”
QQSDILYT

SEQ ID NO: 247 - protein, 9 aa, Artificial Sequence
  source: /note=“Description of Artificial Sequence Synthetic peptide”
GTFGNYAIS

SEQ ID NO: 248 - protein, 17 aa, Artificial Sequence
  source: /note=“Description of Artificial Sequence Synthetic peptide”
GIIPIPGIANYAQKFQG

SEQ ID NO: 249 - protein, 16 aa, Artificial Sequence
  source: /note=“Description of Artificial Sequence Synthetic peptide”
ARDTGRGYTRHFWFDP

SEQ ID NO: 250 - protein, 11 aa, Artificial Sequence
  source: /note=“Description of Artificial Sequence Synthetic peptide”
RASQSISSYLN

SEQ ID NO: 251 - protein, 7 aa, Artificial Sequence
  source: /note=“Description of Artificial Sequence Synthetic peptide”
AASSLQS

SEQ ID NO: 252 - protein, 8 aa, Artificial Sequence
  source: /note=“Description of Artificial Sequence Synthetic peptide”
QQSDILYT

SEQ ID NO: 253 - protein, 9 aa, Artificial Sequence
  source: /note=“Description of Artificial Sequence Synthetic peptide”
GTFSSAAIS

SEQ ID NO: 254 - protein, 17 aa, Artificial Sequence
  source: /note=“Description of Artificial Sequence Synthetic peptide”
GIFPISGHANYAQKFQG

SEQ ID NO: 255 - protein, 16 aa, Artificial Sequence
  source: /note=“Description of Artificial Sequence Synthetic peptide”
ARDTGRGYTRHFWFDP

SEQ ID NO: 256 - protein, 11 aa, Artificial Sequence
  source: /note=“Description of Artificial Sequence Synthetic peptide”
RASQSISSYLN

SEQ ID NO: 257 - protein, 7 aa, Artificial Sequence
  source: /note=“Description of Artificial Sequence Synthetic peptide”
AASSLQS

SEQ ID NO: 258 - protein, 8 aa, Artificial Sequence
  source: /note=“Description of Artificial Sequence Synthetic peptide”
QQSDILYT

SEQ ID NO: 259 - protein, 9 aa, Artificial Sequence
  source: /note=“Description of Artificial Sequence Synthetic peptide”
GTFATYAIS

SEQ ID NO: 260 - protein, 17 aa, Artificial Sequence
  source: /note=“Description of Artificial Sequence Synthetic peptide”
GIFPLSGTANYAQKFQG

SEQ ID NO: 261 - protein, 16 aa, Artificial Sequence
  source: /note=“Description of Artificial Sequence Synthetic peptide”
ARDTGRGYTRHFWFDP

SEQ ID NO: 262 - protein, 11 aa, Artificial Sequence
  source: /note=“Description of Artificial Sequence Synthetic peptide”
RASQSISSYLN

SEQ ID NO: 263 - protein, 7 aa, Artificial Sequence
  source: /note=“Description of Artificial Sequence Synthetic peptide”
AASSLQS

SEQ ID NO: 264 - protein, 8 aa, Artificial Sequence
  source: /note=“Description of Artificial Sequence Synthetic peptide”
QQSDILYT

SEQ ID NO: 265 - protein, 123 aa, Artificial Sequence
  source: /note=“Description of Artificial Sequence Synthetic polypeptide”
EVQLVESGGGLVKPGGSLRLSCAASGFTFSEYTMNWVRQAPGKGLEWVSAIVGSGDSTYYADSVKGRFTISRDNSKNTLYLQMNSLRAEDTAVYYCAKDYSSGDWIDYGMDVWGQGTTVTVSS

SEQ ID NO: 266 - protein, 107 aa, Artificial Sequence
  source: /note=“Description of Artificial Sequence Synthetic polypeptide”
DIQMTQSPSSLSASVGDRVTITCQASQDISNYLNWYQQKPGKAPKLLIYDASNLATGVPSRFSGSGSGTDFTFTISSLQPEDIATYYCQQFDLLPPTFGGGTKVEIK

SEQ ID NO: 267 - protein, 123 aa, Artificial Sequence
  source: /note=“Description of Artificial Sequence Synthetic polypeptide”
EVQLLESGGGLVQPGGSLRLSCAASGFTFSDYAMIWVRQAPGKGLEWVSAISGGGESTYYADSVKGRFTISRDNSKNTLYLQMNSLRAEDTAVYYCAKDYSSGDWIDYGMDVWGQGTTVTVSS

SEQ ID NO: 268 - protein, 107 aa, Artificial Sequence; identical to SEQ ID NO: 266
  source: /note=“Description of Artificial Sequence Synthetic polypeptide”

SEQ ID NO: 269 - protein, 123 aa, Artificial Sequence
  source: /note=“Description of Artificial Sequence Synthetic polypeptide”
QVQLVQSGAEVKKPGSSVKVSCKASGGTFDNYYISWVRQAPGQGLEWMGGIFPIFGTANYAQKFQGRVTITADESTSTAYMELSSLRSEDTAVYYCAREVGHYSGSPYYMDVWGKGTTVTVSS

SEQ ID NO: 270 - protein, 106 aa, Artificial Sequence
  source: /note=“Description of Artificial Sequence Synthetic polypeptide”
DIQMTQSPSTLSASVGDRVTITCRASQSINSWLAWYQQKPGKAPKLLISDASSLESGVPSRFSGSGSGTEFTLTISSLQPDDFATYYCQQVGPYLTFGGGTKVEIK

SEQ ID NO: 271 - protein, 122 aa, Artificial Sequence
  source: /note=“Description of Artificial Sequence Synthetic polypeptide”
EVQLVESGGGLVQPGRSLRLSCTASGFTFGDYAMSWFRQAPGKGLEWVGFIGSKFYGGETEYTASVKGRFTISRDGSKSIAYLQMNSLKTEDTAVYYCARGPRRYTYGMDVWGQGTTVTVSS

SEQ ID NO: 272 - protein, 106 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSSTPLTFGGGTKVEIK

SEQ ID NO: 273 - protein, 120 aa, Artificial Sequence
  source: /note=“Description of Artificial Sequence Synthetic polypeptide”
EVQLVESGGGLVQPGRSLRLSCAASGFTFDDYAVHWVRQAPGKGLEWVSGISWSSGLIGYADSVKGRFTISRDNAKNSLYLQMNSLRAEDTAVYYCAKGPPTYQDYFDLWGRGTLVTVSS

SEQ ID NO: 274 - protein, 108 aa, Artificial Sequence
  source: /note=“Description of Artificial Sequence Synthetic polypeptide”
EIVLTQSPATLSLSPGERATLSCRASQSVSRYLAWYQQKPGQAPRLLIYDASNRATGIPARFSGSGSGTDFTLTISSLEPEDFAVYYCQQVSFFPPITFGGGTKVEIK

SEQ ID NO: 275 - protein, 126 aa, Artificial Sequence
  source: /note=“Description of Artificial Sequence Synthetic polypeptide”
EVQLLESGGGLVQPGGSLRLSCAASGFTFSGHLMSWVRQAPGKGLEWVSAISGSAGETYYADSVKGRFTISRDNSKNTLYLQMNSLRAEDTAVYYCARDAYYDDWSGWADWYFDLWGRGTLVTVSS

SEQ ID NO: 276 - protein, 107 aa, Artificial Sequence
  source: /note=“Description of Artificial Sequence Synthetic polypeptide”
EIVLTQSPATLSLSPGERATLSCRASQSVSRYLAWYQQKPGQAPRLLIYDASNRATGIPARFSGSGSGTDFTLTISSLEPEDFAVYYCQQVSLLPPTFGGGTKVEIK

SEQ ID NO: 277 - protein, 126 aa, Artificial Sequence
  source: /note=“Description of Artificial Sequence Synthetic polypeptide”
EVQLVESGGGLVQPGRSLRLSCTASGFTFGDVAMSWFRQAPGKGLEWVGYIGSKAYGGETEYTASVKGRFTISRDGSKSIAYLQMNSLKTEDTAVYYCARAGHSYGSIASNWFDPWGQGTLVTVSS

SEQ ID NO: 278 - protein, 107 aa, Artificial Sequence
  source: /note=“Description of Artificial Sequence Synthetic polypeptide”
DIQMTQSPSSLSASVGDRVTITCRASQSISSYLNWYQQKPGKAPKLLIYGASSLQSGVPSRFSGSGSGTDFTLTISSLQPEDFATYYCQQGFYTPWTFGGGTKVEIK

SEQ ID NO: 279 - protein, 123 aa, Artificial Sequence
  source: /note=“Description of Artificial Sequence Synthetic polypeptide”
QVQLVQSGAEVKKPGSSVKVSCKASGGTFSSAAISWVRQAPGQGLEWMGNIIPIVGIANYAQKFQGRVTITADESTSTAYMELSSLRSEDTAVYYCARDTGRGYTRHFWFDPWGQGTLVTVSS

SEQ ID NO: 280 - protein, 106 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSDILYTFGGGTKVEIK

SEQ ID NO: 281 - protein, 125 aa, Artificial Sequence
  source: /note=“Description of Artificial Sequence Synthetic polypeptide”
EVQLVQSGAEVKKPGSSVKVSCKASGGTFSNYAISWVRQAPGQGLEWMGGIIPIFGTANYAQKFQGRVTITADESTSTAYMELSSLRSEDTAVYYCARGRGALALVGPYYGMDVWGQGTTVTVSS

SEQ ID NO: 282 - protein, 111 aa, Artificial Sequence
  source: /note=“Description of Artificial Sequence Synthetic polypeptide”
DIVMTQSPLSLPVTPGEPASISCRSSQSLLHSNGYNYLDWYLQKPGQSPQLLIYLGSHRASGVPDRFSGSGSGTDFTLKISRVEAEDVGVYYCMQALRAPTFGGGTKVEIK

SEQ ID NO: 283 - protein, 122 aa, Artificial Sequence
  source: /note=“Description of Artificial Sequence Synthetic polypeptide”
EVQLVESGGGLVQPGRSLRLSCTASGFTFGDYAMSWFRQAPGKGLEWVGFIGSKAYGGTTEYTASVKGRFTISRDGSKSIAYLQMNSLKTEDTAVYYCARGPRRYTYGMDVWGQGTTVTVSS

SEQ ID NO: 284 - protein, 106 aa, Artificial Sequence; identical to SEQ ID NO: 272
  source: /note=“Description of Artificial Sequence Synthetic polypeptide”

SEQ ID NO: 285 - protein, 123 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPISGTANYAQKFQGRVTITADESTSTAYMELSSLRSEDTAVYYCARDTGRGYTRHFWFDPWGQGTLVTVSS

SEQ ID NO: 286 - protein, 106 aa, Artificial Sequence; identical to SEQ ID NO: 280
  source: /note=“Description of Artificial Sequence Synthetic polypeptide”

SEQ ID NO: 287 - protein, 123 aa, Artificial Sequence
  source: /note=“Description of Artificial Sequence Synthetic polypeptide”
QVQLVQSGAEVKKPGSSVKVSCKASGGTFGNYAISWVRQAPGQGLEWMGGIIPIPGIANYAQKFQGRVTITADESTSTAYMELSSLRSEDTAVYYCARDTGRGYTRHFWFDPWGQGTLVTVSS

SEQ ID NO: 288 - protein, 106 aa, Artificial Sequence; identical to SEQ ID NO: 280
  source: /note=“Description of Artificial Sequence Synthetic polypeptide”

SEQ ID NO: 289 - protein, 123 aa, Artificial Sequence
  source: /note=“Description of Artificial Sequence Synthetic polypeptide”
QVQLVQSGAEVKKPGSSVKVSCKASGGTFSSAAISWVRQAPGQGLEWMGGIFPISGHANYAQKFQGRVTITADESTSTAYMELSSLRSEDTAVYYCARDTGRGYTRHFWFDPWGQGTLVTVSS

SEQ ID NO: 290 - protein, 106 aa, Artificial Sequence; identical to SEQ ID NO: 280
  source: /note=“Description of Artificial Sequence Synthetic polypeptide”

SEQ ID NO: 291 - protein, 123 aa, Artificial Sequence
  source: /note=“Description of Artificial Sequence Synthetic polypeptide”
QVQLVQSGAEVKKPGSSVKVSCKASGGTFATYAISWVRQAPGQGLEWMGGIFPLSGTANYAQKFQGRVTITADESTSTAYMELSSLRSEDTAVYYCARDTGRGYTRHFWFDPWGQGTLVTVSS

SEQ ID NO: 292 - protein, 106 aa, Artificial Sequence; identical to SEQ ID NO: 280
  source: /note=“Description of Artificial Sequence Synthetic polypeptide”

SEQ ID NO: 293 - protein, 8 aa, Artificial Sequence
  source: /note=“Description of Artificial Sequence Synthetic peptide”
GYTFTDYN

SEQ ID NO: 294 - protein, 8 aa, Artificial Sequence
  source: /note=“Description of Artificial Sequence Synthetic peptide”
IYPYIGGS

SEQ ID NO: 295 - protein, 13 aa, Artificial Sequence
  source: /note=“Description of Artificial Sequence Synthetic peptide”
AREDKTARNAMDY

SEQ ID NO: 296 - protein, 6 aa, Artificial Sequence
  source: /note=“Description of Artificial Sequence Synthetic peptide”
ENIYSN

SEQ ID NO: 297 - protein, 3 aa, Artificial Sequence
  source: /note=“Description of Artificial Sequence Synthetic peptide”
EAT

SEQ ID NO: 298 - protein, 9 aa, Artificial Sequence
  source: /note=“Description of Artificial Sequence Synthetic peptide”
QHFWGTPYT

SEQ ID NO: 299 - protein, 120 aa, Artificial Sequence
  source: /note=“Description of Artificial Sequence Synthetic polypeptide”
QVQLVQSGAEVKKPGASVKVSCKASGYTFTDYNINWVRQAPGQGLEWMGYIYPYIGGSGYAQKFQGRVTMTRDTSTSTVYMELSSLRSEDTAVYYCAREDKTARNAMDYWGQGTLVTVSS

SEQ ID NO: 300 - protein, 107 aa, Artificial Sequence
  source: /note=“Description of Artificial Sequence Synthetic polypeptide”
DIQMTQSPSSLSASVGDRVTITCRVSENIYSNLAWYQQKPGKAPKLLIYEATNLAEGVPSRFSGSGSGTDFTLTISSLQPEDFATYYCQHFWGTPYTFGQGTKLEIK

SEQ ID NO: 301 - protein, 119 aa, Artificial Sequence
  source: /note=“Description of Artificial Sequence Synthetic polypeptide”
QVQLVQSGAEVKKPGASVKVSCKASGYAFTNYLIEWVRQAPGQGLEWIGVINPGSGGIYYAQKFQGRVTMTADTSTSTVYMELSSLRSEDTAVYYCARSETHDTWFAYWGQGTLVTVSS

SEQ ID NO: 302 - protein, 107 aa, Artificial Sequence
  source: /note=“Description of Artificial Sequence Synthetic polypeptide”
DIQMTQSPSSLSASVGDRVTITCKASQSVRIAVAWFQQKPGKAPKALIYLASTRHTGVPSRFSGSGSGTDFTLTISSVQPEDFATYYCLQHWNYPYTFGQGTKLEIK

SEQ ID NO: 303 - protein, 9 aa, Artificial Sequence
  source: /note=“Description of Artificial Sequence Synthetic peptide”
GTFSSAAIS

SEQ ID NO: 304 - protein, 17 aa, Artificial Sequence
  source: /note=“Description of Artificial Sequence Synthetic peptide”
NIIPIVGIANYAQKFQG

SEQ ID NO: 305 - protein, 16 aa, Artificial Sequence
  source: /note=“Description of Artificial Sequence Synthetic peptide”
ARDTGRGYTRHFWFDP

SEQ ID NO: 306 - protein, 11 aa, Artificial Sequence
  source: /note=“Description of Artificial Sequence Synthetic peptide”
RASQSISSYLN

SEQ ID NO: 307 - protein, 7 aa, Artificial Sequence
  source: /note=“Description of Artificial Sequence Synthetic peptide”
AASSLQS

SEQ ID NO: 308 - protein, 8 aa, Artificial Sequence
  source: /note=“Description of Artificial Sequence Synthetic peptide”
QQSDILYT

SEQ ID NO: 309 - protein, 123 aa, Artificial Sequence; identical to SEQ ID NO: 279
  source: /note=“Description of Artificial Sequence Synthetic polypeptide”

SEQ ID NO: 310 - protein, 106 aa, Artificial Sequence; identical to SEQ ID NO: 280
  source: /note=“Description of Artificial Sequence Synthetic polypeptide”

SEQ ID NO: 311 - protein, 220 aa, Artificial Sequence
  source: /note=“Description of Artificial Sequence Synthetic polypeptide”
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 312 - protein, 456 aa, Artificial Sequence
  source: /note=“Description of Artificial Sequence Synthetic polypeptide”
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 114 aa, Artificial Sequence
  source: /note=“Description of Artificial Sequence Synthetic polypeptide”
DIVMTQSPSSLAVSPGEKVTMTCKSSQSLYYSGVKENLLAWYQQKPGQSPKLLIYYASIRFTGVPDRFTGSGSGTDYTLTITSVQAEDMGQYFCQQGINNPLTFGDGTKLEIKR

SEQ ID NO: 314 - protein, 112 aa, Artificial Sequence
  source: /note=“Description of Artificial Sequence Synthetic polypeptide”
DVVLTQTPLSLSVSFGDQVSISCRSSQSLVNSYGNTFLSWYLHKPGQSPQLLIFGISNRFSGVPDRFSGSGSGTDFTLKISTIKPEDLGMYYCLQGTHQPPTFGPGTKLEVK

SEQ ID NO: 315 - protein, 119 aa, Artificial Sequence
  source: /note=“Description of Artificial Sequence Synthetic polypeptide”
EVQLVESGGGLTQPGKSLKLSCEASGFTFSSFTMHWVRQSPGKGLEWVAFIRSGSGIVFYADAVRGRFTISRDNAKNLLFLQMNDLKSEDTAMYYCARRPLGHNTFDSWGQGTLVTVSS

SEQ ID NO: 316 - protein, 119 aa, Artificial Sequence
  source: /note=“Description of Artificial Sequence Synthetic polypeptide”
EVQLQQSGPELVKPGTSMKISCKASGYSFTGHLMNWVKQSHGKNLEWIGLIIPYNGGTSYNQKFKGKATLTVDKSSSTAYMELLSLTSDDSAVYFCSRGLRGFYAMDYWGQGTSVTVSS

SEQ ID NO: 317 - protein, 17 aa, Artificial Sequence
  source: /note=“Description of Artificial Sequence Synthetic peptide”
KSSQSLYYSGVKENLLA

SEQ ID NO: 318 - protein, 6 aa, Artificial Sequence
  source: /note=“Description of Artificial Sequence Synthetic peptide”
ASIRFT

SEQ ID NO: 319 - protein, 9 aa, Artificial Sequence
  source: /note=“Description of Artificial Sequence Synthetic peptide”
QQGINNPLT

SEQ ID NO: 320 - protein, 10 aa, Artificial Sequence
  source: /note=“Description of Artificial Sequence Synthetic peptide”
GFTFSSFTMH

SEQ ID NO: 321 - protein, 17 aa, Artificial Sequence
  source: /note=“Description of Artificial Sequence Synthetic peptide”
FIRSGSGIVFYADAVRG

SEQ ID NO: 322 - protein, 10 aa, Artificial Sequence
  source: /note=“Description of Artificial Sequence Synthetic peptide”
RPLGHNTFDS

SEQ ID NO: 323 - protein, 16 aa, Artificial Sequence
  source: /note=“Description of Artificial Sequence Synthetic peptide”
RSSQSLVNSYGNTFLS

SEQ ID NO: 324 - protein, 7 aa, Artificial Sequence
  source: /note=“Description of Artificial Sequence Synthetic peptide”
GISNRFS

SEQ ID NO: 325 - protein, 9 aa, Artificial Sequence
  source: /note=“Description of Artificial Sequence Synthetic peptide”
LQGTHQPPT

SEQ ID NO: 326 - protein, 10 aa, Artificial Sequence
  source: /note=“Description of Artificial Sequence Synthetic peptide”
GYSFTGHLMN

SEQ ID NO: 327 - protein, 17 aa, Artificial Sequence
  source: /note=“Description of Artificial Sequence Synthetic peptide”
LIIPYNGGTSYNQKFKG

SEQ ID NO: 328 - protein, 10 aa, Artificial Sequence
  source: /note=“Description of Artificial Sequence Synthetic peptide”
GLRGFYAMDY

SEQ ID NO: 329 - protein, 119 aa, Artificial Sequence
  source: /note=“Description of Artificial Sequence Synthetic polypeptide”
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 330 - protein, 107 aa, Artificial Sequence
  source: /note=“Description of Artificial Sequence Synthetic polypeptide”
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 331 - protein, 11 aa, Artificial Sequence
  source: /note=“Description of Artificial Sequence Synthetic peptide”
RASEHIYSYLS

SEQ ID NO: 332 - protein, 7 aa, Artificial Sequence
  source: /note=“Description of Artificial Sequence Synthetic peptide”
NAKTLAE

SEQ ID NO: 333 - protein, 9 aa, Artificial Sequence
  source: /note=“Description of Artificial Sequence Synthetic peptide”
QHHFGSPLT

SEQ ID NO: 334 - protein, 5 aa, Artificial Sequence
  source: /note=“Description of Artificial Sequence Synthetic peptide”
SYVMH

SEQ ID NO: 335 - protein, 17 aa, Artificial Sequence
  source: /note=“Description of Artificial Sequence Synthetic peptide”
YIDPYNDGAKYAQKFQG

SEQ ID NO: 336 - protein, 10 aa, Artificial Sequence
  source: /note=“Description of Artificial Sequence Synthetic peptide”
GGPYGWYFDV

SEQ ID NO: 337 - protein, 119 aa, Artificial Sequence
  source: /note=“Description of Artificial Sequence Synthetic polypeptide”
EVQLQQSGPELVKPGSSVKMSCKASGYTFSSYVMHWVKQKPGQGLEWIGYIDPYNDGAKYNEKFKGKATLTSDKSSSTAYMELSSLTSEDSAVYYCARGGPYGWYFDVWGAGTTVTVSS

SEQ ID NO: 338 - protein, 107 aa, Artificial Sequence
  source: /note=“Description of Artificial Sequence Synthetic polypeptide”
DIQMTQSPASLSASVGETVTITCRASEHIYSYLSWYQQKQGKSPQLLVYNAKTLAEGVPSRFSGSGSGTQFSLKINSLQPEDFGTYYCQHHFGSPLTFGAGTTLELK

SEQ ID NO: 339 - protein, 127 aa, Artificial Sequence
  source: /note=“Description of Artificial Sequence Synthetic polypeptide”
EVQLQESGPGLVKPSQSLSLTCSVTGSSIASDYWGWIRKFPGNKMEWMGFITYSGSTSYNPSLKSRISITRDTSKNQFFLQLHSVTTDDTATYSCARMPSFITLASLSTWEGYFDFWGPGTMVTVSS

SEQ ID NO: 340 - protein, 106 aa, Artificial Sequence
  source: /note=“Description of Artificial Sequence Synthetic polypeptide”
DIQMTQSPSLLSASVGDRVTLNCKASQSIHKNLAWYQQKLGEAPKFLIYYANSLQTGIPSRFSGSGSGTDFTLTISGLQPEDVATYFCQQYYSGWTFGGGTKVELK

SEQ ID NO: 341 - protein, 121 aa, Artificial Sequence
  source: /note=“Description of Artificial Sequence Synthetic polypeptide”
DVQLQESGPGLVKPSQSLSLTCTVTGYSITSDYAWNWIRQFPGNKLEWMGYISNSGSASYNPSLKSRISITRDTSKNQFFLQLNSVTTEDTATYYCATLIYYDYGGAMNFWGQGTSVTVSS

SEQ ID NO: 342 - protein, 107 aa, Artificial Sequence
  source: /note=“Description of Artificial Sequence Synthetic polypeptide”
DIVMTQSHKFMSTSVGDRVSITCKASQGVSTTVAWYQQKPGQSPKLLIYSASYRYTGVPDRFTGSGSGTDFTFTISSVQSEDLAVYYCQHYYSTPWTFGGGTKLEIK

SEQ ID NO: 343 - protein, 130 aa, Artificial Sequence
  source: /note=“Description of Artificial Sequence Synthetic polypeptide”
QVHLQESGPGLVKPSETLSLTCTVSGGSVSSGIYYWSWIRQPPGKGLEWIGYIYYSGSTNYNPSLKSRVTISVDTSKNQFSLKLSSVTAADTAVYYCARDYYVSGNYYNVDYYFFGVDVWGQGTTVTVSS

SEQ ID NO: 344 - protein, 130 aa, Artificial Sequence
  source: /note=“Description of Artificial Sequence Synthetic polypeptide”
QVQLQESGPGLVKPSETLSLTCTVSGGSVSSGIYYWSWIRQPPGKGLEWIGYIYYSGSTNYNPSLKSRVTISVDTSKNQFSLKLSSVTAADTAVYYCARDYYVSGNYYNVDYYFFGVDVWGQGTTVTVSS

SEQ ID NO: 345 - protein, 109 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLFTFGPGTKVDIK

SEQ ID NO: 346 - protein, 11 aa, Artificial Sequence
  source: /note=“Description of Artificial Sequence Synthetic peptide”
RASQSVSSYLA

SEQ ID NO: 347 - protein, 7 aa, Artificial Sequence
  source: /note=“Description of Artificial Sequence Synthetic peptide”
DASNRAT

SEQ ID NO: 348 - protein, 11 aa, Artificial Sequence
  source: /note=“Description of Artificial Sequence Synthetic peptide”
QQRSNWPPLFT

SEQ ID NO: 349 - protein, 7 aa, Artificial Sequence
  source: /note=“Description of Artificial Sequence Synthetic peptide”
SGIYYWS

SEQ ID NO: 350 - protein, 16 aa, Artificial Sequence
  source: /note=“Description of Artificial Sequence Synthetic peptide”
YIYYSGSTNYNPSLKS

SEQ ID NO: 351 - protein, 16 aa, Artificial Sequence
  source: /note=“Description of Artificial Sequence Synthetic peptide”
DYYVSGNYYNVDYYFF

SEQ ID NO: 352 - protein, 127 aa, Artificial Sequence
  source: /note=“Description of Artificial Sequence Synthetic polypeptide”
EVQLVESGGGLVQPGGSLRLSCAASGFTFSSYGMSWVRQAPGKGLEWVANIKQDGSEKYYVDSVKGRFTISRDNAKNSLYLQMNSLRAEDTAVYYCARVSYYYDSSKLRWAEYFQHWGQGTLVTVSS

SEQ ID NO: 353 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASKRATGIPARFSGSGSGTDFTLTISSLEPEDFAVYYCQQVHNFPLTFGGGTKVEIK

SEQ ID NO: 354 - protein, 126 aa, Artificial Sequence
  source: /note=“Description of Artificial Sequence Synthetic polypeptide”
EVQLVESGGGWQPGRSLRLSCAASGFTFESYGMVWVRQAPGKGLEWVASILYDGSNRYYADSVKGRFTVSRDNSKNTLYLQMNSLRAEDTAVYYCARVSYYYDSVELRWAEYFQHWGQGTLVTVSS

SEQ ID NO: 355 - protein, 107 aa, Artificial Sequence; identical to SEQ ID NO: 353
  source: /note=“Description of Artificial Sequence Synthetic polypeptide”

SEQ ID NO: 356 - protein, 121 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGVINPSGGSTSYAQKFQGRVTMTRDTSTSTVYMELSSLRSEDTAVYYCARDHSDYWSGILDVWGQGTMVTVSS

SEQ ID NO: 357 - protein, 108 aa, Artificial Sequence
  source: /note=“Description of Artificial Sequence Synthetic polypeptide”
EIVLTQSPGTLSLSPGERATLSCRASQSVRSSYLAWYQQKPGQAPRLLIYGASSRATGIPDRFSGSGSGTDFTLTISRLEPEDFAVYYCQQYFSPPWTFGGGTKVEIK

SEQ ID NO: 358 - protein, 121 aa, Artificial Sequence
  source: /note=“Description of Artificial Sequence Synthetic polypeptide”
QVQLVQSGAEVKKPGASVKVSCKASGYTFEKYYMHWVRQAPGQGLEWMGVIGPSGASTSYAQKFQGRVTMTRDTSTSTVYMELSSLRSEDTAVYYCARDHSDYWSGILHSWGQGTMVTVSS

SEQ ID NO: 359 - protein, 108 aa, Artificial Sequence; identical to SEQ ID NO: 357
  source: /note=“Description of Artificial Sequence Synthetic polypeptide”

SEQ ID NO: 360 - protein, 121 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGVIGPSGASTSYAQKFQGRVTLTRDTSTSTVYMELSSLRSEDTAVYYCARDHSDYWSGIMEVWGQGTMVTVSS

SEQ ID NO: 361 - protein, 108 aa, Artificial Sequence; identical to SEQ ID NO: 357
  source: /note=“Description of Artificial Sequence Synthetic polypeptide”

SEQ ID NO: 362 - protein, 121 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGVIGPSGASTSYAQKFQGRVTLTRDTSTSTVYMELSSLRSEDTAVYYCARDHSDYWSGIMEVWGQGTTVTVSS

SEQ ID NO: 363 - protein, 108 aa, Artificial Sequence; identical to SEQ ID NO: 357
  source: /note=“Description of Artificial Sequence Synthetic polypeptide”

SEQ ID NO: 364 - protein, 124 aa, Artificial Sequence
  source: /note=“Description of Artificial Sequence Synthetic polypeptide”
QVQLQESGPGLVKPSETLSLTCAVSGYSISSGYYWAWIRQPPGKGLEWIGSIYHSGSTYYNPSLKSRVTISVDTSKNQFSLKLSSVTAADTAVYYCAIEGANYYDFGYVAFDIWGQGTMVTVSS

SEQ ID NO: 365 - protein, 107 aa, Artificial Sequence
  source: /note=“Description of Artificial Sequence Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQSPPWPRTFGGGTKVEIK

SEQ ID NO: 366 - protein, 125 aa, Artificial Sequence
  source: /note=“Description of Artificial Sequence Synthetic polypeptide”
QVQLQESGPGLVKPSETLSLTCTVSGGSISSGSYYLAWIRQPPGKGLEWIGSIFRSGSTYYNPSLESRVTISVDTSKNQFSLKLSSVTAADTAVYYCAIEGANFKDFGYVAFDIWGQGTMVTVSS

SEQ ID NO: 367 - protein, 107 aa, Artificial Sequence; identical to SEQ ID NO: 365
  source: /note=“Description of Artificial Sequence Synthetic polypeptide”

SEQ ID NO: 368 - protein, 125 aa, Artificial Sequence
  source: /note=“Description of Artificial Sequence Synthetic polypeptide”
QLQLQESGPGLVKPSETLSLTCTVSGGSISSSRYYWAWIRQPPGKGLEWIGSIGTSGSTYYNPSLKSRVTISVDTSKNQFSLKLSSVTATDTAVYYCAIEGANFRDFGYVAFDIWGQGTMVTVSS

SEQ ID NO: 369 - protein, 107 aa, Artificial Sequence; identical to SEQ ID NO: 365
  source: /note=“Description of Artificial Sequence Synthetic polypeptide”

SEQ ID NO: 370 - protein, 122 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIIPIFGTANYAQKFQGRVTITADESTSTAYMELSSLRSEDTAVYYCARLHLGSSAYYGMDVWGQGTTVTVSS

SEQ ID NO: 371 - protein, 107 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQRYVFPPTFGGGTKVEIK

SEQ ID NO: 372 - protein, 122 aa, Artificial Sequence
  source: /note=“Description of Artificial Sequence Synthetic polypeptide”
QVQLVQSGAEVKKPGSSVKVSCKASGGTFQNYAISVWRQAPGQGLEWMGVIVPIFGTANYAQKFQGRVTVTADESTSTAYMELSSLRSEDTAVYYCARLHLGQKAYYGMDVWGQGTTVTVSS

SEQ ID NO: 373 - protein, 107 aa, Artificial Sequence; identical to SEQ ID NO: 371
  source: /note=“Description of Artificial Sequence Synthetic polypeptide”

SEQ ID NO: 374 - protein, 119 aa, Artificial Sequence
  source: /note=“Description of Artificial Sequence Synthetic polypeptide”
EVQLVESGGGLVQPGRSLRLSCAASGFTFGDYAMHVWRQAPGKGLEVWSGITWNSGSIGYADSVKGRFTISRDNAKNSLYLQMNSLRAEDTALYYCAKPVPKSRGLDVWGQGTMVTVSS

SEQ ID NO: 375 - protein, 107 aa, Artificial Sequence
  source: /note=“Description of Artificial Sequence Synthetic polypeptide”
DIQLTQSPSSVSASVGDRVTITCRASQGISSWLAWYQQKPGKAPKLLIYGASSLQSGVPSRFSGSGSGTDFTLTISSLQPEDFATYYCQQAFYLPWTFGGGTKVEIK

SEQ ID NO: 376 - protein, 119 aa, Artificial Sequence
  source: /note=“Description of Artificial Sequence Synthetic polypeptide”
QVQLVESGGGLVQPGRSLRLSCAASGFTFRDYAMHVWRQAPGKGLEVWSGITWNSGLIGYADSVKGRFTISRDNAKNSLYLQMNSLRAEDTALYYCAKPVPRLRGLDVWGQGTMVTVSS

SEQ ID NO: 377 - protein, 107 aa, Artificial Sequence; identical to SEQ ID NO: 375
  source: /note=“Description of Artificial Sequence Synthetic polypeptide”

SEQ ID NO: 378 - protein, 119 aa, Artificial Sequence
  source: /note=“Description of Artificial Sequence Synthetic polypeptide”
EVQLVESGGGVVQPGRSLRLSCAASGFTFGSYYMHWVRQAPGKGLEVWAVIWPDGSNKLYADSVKGRFTISRDNSKNTLYLQMNSLRAEDTALYYCAKPVPKSRALDVWGQGTMVTVSS

SEQ ID NO: 379 - protein, 107 aa, Artificial Sequence; identical to SEQ ID NO: 375
  source: /note=“Description of Artificial Sequence Synthetic polypeptide”

SEQ ID NO: 380 - protein, 119 aa, Artificial Sequence
  source: /note=“Description of Artificial Sequence Synthetic polypeptide”
QVQLVESGGGVVQPGRSLRLSCAASGFTFSSSYMHWVRQAPGKGLEVWAVIGADGSNKYYADSVEGRFTISRDNSKNTLYLQMNSLRAEDTALYYCAKPVPRRRGLDVWGQGTMVTVSS

SEQ ID NO: 381 - protein, 107 aa, Artificial Sequence; identical to SEQ ID NO: 375
  source: /note=“Description of Artificial Sequence Synthetic polypeptide”

SEQ ID NO: 382 - protein, 125 aa, Artificial Sequence
  source: /note=“Description of Artificial Sequence Synthetic polypeptide”
QLQLQESGPGLVKPSETLSLTCTVSGGSISSGSYYLAWIRQPPGKGLEWIGSIFRSGSTYYNPSLESRVTISVDTSKNQFSLKLSSVTAADTAVYYCAIEGANFKDFGYVAFDIWGQGTTVTVSS

SEQ ID NO: 383 - protein, 107 aa, Artificial Sequence; identical to SEQ ID NO: 365
  source: /note=“Description of Artificial Sequence Synthetic polypeptide”

SEQ ID NO: 384 - protein, 125 aa, Artificial Sequence; identical to SEQ ID NO: 382
  source: /note=“Description of Artificial Sequence Synthetic polypeptide”

SEQ ID NO: 385 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQENPRPRTFGGGTKVEIK

SEQ ID NO: 386 - protein, 125 aa, Artificial Sequence; identical to SEQ ID NO: 382
  source: /note=“Description of Artificial Sequence Synthetic polypeptide”

SEQ ID NO: 387 - protein, 107 aa, Artificial Sequence
  source: /note=“Description of Artificial Sequence Synthetic polypeptide”
EIVMTQSPATLSVSPGERATLSCRASKSVSSNLAWYQQKPGQAPRLLIYFASTRATGIPARFSGSGSGTEFTLTISSLQSEDFAVYYCQQTSPWPRTFGGGTKVEIK

SEQ ID NO: 388 - protein, 120 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREAQSYRVPFDIWGQGTMVTVSS

SEQ ID NO: 389 - protein, 108 aa, Artificial Sequence
  source: /note=“Description of Artificial Sequence Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQQYAIWPPFTFGGGTKVEIK

SEQ ID NO: 390 - protein, 120 aa, Artificial Sequence
  source: /note=“Description of Artificial Sequence Synthetic polypeptide”
QVQLVQSGAEVKKPGSSVKVSCKASGGTFSSYLISWVRQAPGQGLEWMGGIYPIFATANYAQKFQGRVTITADESTSTAYMELSSLRSEDTAVYYCAREAQSYRVPFDIWGQGTMVTVSS

SEQ ID NO: 391 - protein, 108 aa, Artificial Sequence; identical to SEQ ID NO: 389
  source: /note=“Description of Artificial Sequence Synthetic polypeptide”

SEQ ID NO: 392 - protein, 453 aa, Artificial Sequence
  source: /note=“Description of Artificial Sequence Synthetic polypeptide”
QVQLQESGPGLVKPSQTLSLTCTVSGGSIESGLYYWGWIRQPPGKGLEWIGSIYYSGSTYYNPSLKSRATISVDTSKNQFSLKLSSVTAADTAVYYCARDGVLALNKRSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 393 - protein, 216 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQHTVRPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 394 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQHTVRPPLTFGGGTKVEIK

SEQ ID NO: 395 - protein, 123 aa, Artificial Sequence
  source: /note=“Description of Artificial Sequence Synthetic polypeptide”
QVQLQESGPGLVKPSQTLSLTCTVSGGSIESGLYYWGWIRQPPGKGLEWIGSIYYSGSTYYNPSLKSRATISVDTSKNQFSLKLSSVTAADTAVYYCARDGVLALNKRSFDIWGQGTMVTVSS

SEQ ID NO: 396 - protein, 5 aa, Artificial Sequence
  source: /note=“Description of Artificial Sequence Synthetic peptide”
DYYMY

SEQ ID NO: 397 - protein, 17 aa, Artificial Sequence
  source: /note=“Description of Artificial Sequence Synthetic peptide”
YITKGGGSTYYPDTVKG

SEQ ID NO: 398 - protein, 10 aa, Artificial Sequence
  source: /note=“Description of Artificial Sequence Synthetic peptide”
QTNYDFTMDY

SEQ ID NO: 399 - protein, 11 aa, Artificial Sequence
  source: /note=“Description of Artificial Sequence Synthetic peptide”
KASQDVGTSVA

SEQ ID NO: 400 - protein, 7 aa, Artificial Sequence
  source: /note=“Description of Artificial Sequence Synthetic peptide”
WASARHT

SEQ ID NO: 401 - protein, 9 aa, Artificial Sequence
  source: /note=“Description of Artificial Sequence Synthetic peptide”
QQYSSYPLT

SEQ ID NO: 402 - protein, 119 aa, Artificial Sequence
  source: /note=“Description of Artificial Sequence Synthetic polypeptide”
EVKLVESGGGLVQPGGSLKLSCATSGFTFSDYYMYWIRQTPEKRLEWVAYITKGGGSTYYPDTVKGRFTISRDNAKNTLYLQVSRLKSEDTAIYYCARQTNYDFTMDYWGQGTSVTVSS

SEQ ID NO: 403 - protein, 107 aa, Artificial Sequence
  source: /note=“Description of Artificial Sequence Synthetic polypeptide”
DIVMTQSHKFMSTSVGDRVSIICKASQDVGTSVAWYQQKPGQSPNLLIYWASARHTGVPDRFTGSGSGTDFTLTISNVQSEDLADYFCQQYSSYPLTFGAGTKLELK

SEQ ID NO: 404 - protein, 121 aa, Artificial Sequence
  source: /note=“Description of Artificial Sequence Synthetic polypeptide”
EVQLVETGGGLIQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSS

SEQ ID NO: 405 - protein, 111 aa, Artificial Sequence
  source: /note=“Description of Artificial Sequence Synthetic polypeptide”
QSALTQPRSASGNPGQRVTISCSGSSSNMGRRPVNWYQQIPGTAPKLLIYSQNQRPSGVPDRFSGSQSGTSASLTISGLQSEDEAEYFCAVWDDIGRVLQLGGGTQLAVLG

SEQ ID NO: 406 - protein, 121 aa, Artificial Sequence
  source: /note=“Description of Artificial Sequence Synthetic polypeptide”
EVQLVETGGGLIQPGRSLRLSCAASGFTFSSYAMHWVRQAPGKGLEWVAVISYDGTPVYYADSVKGRFTISRDNSKNTLYLQMNSLRAEDTAVYYCARDPLPLHYYGMDVWGQGTTVTVSS

SEQ ID NO: 407 - protein, 111 aa, Artificial Sequence
  source: /note=“Description of Artificial Sequence Synthetic polypeptide”
QSALTQPRSASGNPGQRVTISCSGSSSNMGRRPVNWYQQIPGTAPKLLIYSQNQRPSGVPDRFSGSQSGTSASLTISGLQSEDEAEYFCAVWDGDRRSLQLGGGTQLAVLG

SEQ ID NO: 408 - protein, 119 aa, Artificial Sequence
  source: /note=“Description of Artificial Sequence Synthetic polypeptide”
EVQLVESGGGLVQPGGSLRLSCAASGFTFSSYIMSWVRQAPGKGLEWVATISGGGTNTYYADSVKGRFTISRDNSKNTLYLQMNSLRAEDTAVYYCAKWLLSYYAMDYWGQGTLVTVSS

SEQ ID NO: 409 - protein, 107 aa, Artificial Sequence
  source: /note=“Description of Artificial Sequence Synthetic polypeptide”
DIQMTQSPSSLSASVGDRITITCRASQNINVWLSWYQQKPGKAPKLLIYKASKSHTGVPSRFSGSGSGTDFTLTISSLQPEDFATYYCQQGQSYPYTFGQGTKLEIK

SEQ ID NO: 410 - protein, 122 aa, Artificial Sequence
  source: /note=“Description of Artificial Sequence Synthetic polypeptide”
QVQLVQSGAEVKKPGASVKVSCKASGYTFTSYYMSWVRQAPGQGLEWMGIINPSGGSTSYAQKFQGRVTMTRDTSTSTVYMELSSLRSEDTAVYYCARARYPSSWPYGMDVWGQGTTVTVSS

SEQ ID NO: 411 - protein, 122 aa, Artificial Sequence
  source: /note=“Description of Artificial Sequence Synthetic polypeptide”
QVQLVQSGAEVKKPGASVKVSCKASGYTFLSYYMNWVRQAPGQGLEWMGIIDPSGGRTSYAQKFQGRVTMTRDTSTSTVYMELSSLRSEDTAVYYCARARYPSSWPYGTDVWGQGTTVTVSS

SEQ ID NO: 412 - protein, 122 aa, Artificial Sequence
  source: /note=“Description of Artificial Sequence Synthetic polypeptide”
QVQLVQSGAEVKKPGASVKVSCKASGYTFRSYYMSWVRQAPGQGLEWMGIIDPSGGRTSFAQKFQGRVTMTRDTSTSTVYMELSSLRSEDTAVYYCARARYPESWPYGMDVWGQGTTVTVSS

SEQ ID NO: 413 - protein, 122 aa, Artificial Sequence
  source: /note=“Description of Artificial Sequence Synthetic polypeptide”
QVQLVQSGAEVKKPGASVKVSCKASGYTFGSYYMGWVRQAPGQGLEWMGIIDPSGGRTSYARKFQGRVTMTRDTSTSTVYMELSSLRSEDTAVYYCARARTPSSWPYGMDVWGQGTTVTVSS

SEQ ID NO: 414 - protein, 122 aa, Artificial Sequence
  source: /note=“Description of Artificial Sequence Synthetic polypeptide”
QLQLQESGPGLVKPSETLSLTCTVSGGSISSSRYYWGWIRQPPGKGLEWIGSIYYSGSTYYNPSLKSRVTISVDTSKNQFSLKLSSVTAADTAVYYCARDGLYHTPEYFQHWGQGTLVTVSS

SEQ ID NO: 415 - protein, 122 aa, Artificial Sequence
  source: /note=“Description of Artificial Sequence Synthetic polypeptide”
QLQLQESGPGLVKPSETLSLTCTVSGGSIGSSQYYWGWIRQPPGKGLEWIGSIYRSGGTYYNPSLKSRVTISVDTSKNQFSLKLSSVTAADTAVYYCARDGLYHTPEYFQHWGQGTLVTVSS

SEQ ID NO: 416 - protein, 122 aa, Artificial Sequence; identical to SEQ ID NO: 414
  source: /note=“Description of Artificial Sequence Synthetic polypeptide”

SEQ ID NO: 417 - protein, 122 aa, Artificial Sequence
  source: /note=“Description of Artificial Sequence Synthetic polypeptide”
EVQLVESGGGLVQPGGSLRLSCAASGFTFSSYSMNWVRQAPGKGLEWVSYISGSSSTIYYADSVKGRFTISRDNAKNSLYLQMNSLRAEDTAVYYCARHRIADSPSRAFDIWGQGTMVTVSS

SEQ ID NO: 418 - protein, 121 aa, Artificial Sequence
  source: /note=“Description of Artificial Sequence Synthetic polypeptide”
EVQLVESGGGLVQPGGSLRLSCAASGFTFSSYSMNWVRQAPGKGLEWVSYISSSGTINYADSVKGRFTISRDNAKNSLYLQMNSLRAEDTAVYYCARHRIADSPSRAFDIWGQGTMVTVSS

SEQ ID NO: 419 - protein, 121 aa, Artificial Sequence
  source: /note=“Description of Artificial Sequence Synthetic polypeptide”
EVQLVESGGGLVQPGGSLRLSCAASGFTIGGYSMNWVRQAPGKGLEWVSYISSSSTIHYADSVKGRFTISRDNAKNSLYLQMNSLRAEDTAVYYCARHRIGRSPSRAFDIWGQGTMVTVSS

SEQ ID NO: 420 - protein, 124 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDPGTDSSGYYYIWRYWGQGTLVTVSS

SEQ ID NO: 421 - protein, 124 aa, Artificial Sequence
  source: /note=“Description of Artificial Sequence Synthetic polypeptide”
EVQLLESGGGLVQPGGSLRLSCAASGFTFSPYGMSWVRQAPGKGLEWVSSISGSGGRTYYADSVKGRFTISRDNSKNTLYLQMNSLRAEDTAVYYCAKDPGTDSSGYYYIWRYWGQGTLVTVSS

SEQ ID NO: 422 - protein, 124 aa, Artificial Sequence
  source: /note=“Description of Artificial Sequence Synthetic polypeptide”
EVQLLESGGGLVQPGGSLRLSCAASGFTFGPYGMSWVRQAPGKGLEWVSAISGSGASTWYADSVKGRFTISRDNSKNTLYLQMNSLRAEDTAVYYCAKDPGTHYSGYYYIWRYWGQGTLVTVSS

SEQ ID NO: 423 - protein, 124 aa, Artificial Sequence
  source: /note=“Description of Artificial Sequence Synthetic polypeptide”
EVQLLESGGGLVQPGGSLRLSCAASGFTFGPYGMSWVRQAPGKGLEWVSAISGSGASTWHADSVKGRFTISRDNSKNTLYLQMNSLRAEDTAVYYCAKDPGTDSTGYYYIWRYWGQGTLVTVSS

SEQ ID NO: 424 - protein, 122 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDHDIAAAGRLADYWGQGTLVTVSS

SEQ ID NO: 425 - protein, 122 aa, Artificial Sequence
  source: /note=“Description of Artificial Sequence Synthetic polypeptide”
QVQLVQSGAEVKKPGASVKVSCKASGYTFTEYYMHWVRQAPGQGLEWMGIISPSAGSTSYAQKFQGRVTMTRDTSTSTVYMELSSLRSEDTAVYYCARDHDIAAAGRLADYWGQGTLVTVSS

SEQ ID NO: 426 - protein, 122 aa, Artificial Sequence
  source: /note=“Description of Artificial Sequence Synthetic polypeptide”
QVQLVQSGAEVKKPGASVKVSCKASGYTFTEYYMHWVRQAPGQGLEWMGIISPSAGSTKYAQKFQGRVTMTRDTSTSTVYMELSSLRSEDTAVYYCARDHDIRLAGRLADYWGQGTLVTVSS

SEQ ID NO: 427 - protein, 123 aa, Artificial Sequence
  source: /note=“Description of Artificial Sequence Synthetic polypeptide”
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAREAGRGTTGLLFDYWGQGTLVTVSS

SEQ ID NO: 428 - protein, 123 aa, Artificial Sequence
  source: /note=“Description of Artificial Sequence Synthetic polypeptide”
QLQLQESGPGLVKPSETLSLTCTVSGGSISSSLYYWGWIRQPPGKGLEWIGSIYYSGSTFYNPSFKSRVTISVDTSKNQFSLKLSSVTAADTAVYYCAREAGRGTTGLLFDYWGQGTLVTVSS

SEQ ID NO: 429 - protein, 123 aa, Artificial Sequence
  source: /note=“Description of Artificial Sequence Synthetic polypeptide”
QLQLQESGPGLVKPSETLSLTCTVSGGSIASSVYYWGWIRQPPGKGLEWIGSIYYSGSTWYNPSLKSRVTISVDTSKNQFSLKLSSVTAADTAVYYCAREAGRTGTGLLFDYWGQGTLVTVSS

SEQ ID NO: 430 - protein, 107 aa, Artificial Sequence
  source: /note=“Description of Artificial Sequence Synthetic polypeptide”
DIQMTQSPSSLSASVGDRVTITCRASQSINSYLNWYQQKPGKAPKLLIYAASSLQSGVPSRFSGSGSGTDFTLTISSLQPEDFATYYCQQSLLTPFTFGGGTKVEIK

SEQ ID NO: 431 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRFAFPYTFGGGTKVEIK

SEQ ID NO: 432 - protein, 107 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQRFAHPYTFGGGTKVEIK

SEQ ID NO: 433 - protein, 112 aa, Artificial Sequence
  source: /note=“Description of Artificial Sequence Synthetic polypeptide”
DIVMTQSPDSLAVSLGERATINCKSSQSVLFSSNNKNYLAWYQQKPGQPPKLLIYWASTRESGVPDRFSGSGSGTDFTLTISSLQAEDVAVYYCQQSYFFPTFGGGTKVEIK

SEQ ID NO: 434 - protein, 108 aa, Artificial Sequence
  source: /note=“Description of Artificial Sequence Synthetic polypeptide”
EIVMTQSPATLSVSPGERATLSCRASQSVSSNLAWYQQKPGQAPRLLIYSASTRATGIPARFSGSGSGTEFTLTISSLQSEDFAVYYCQQYVPHPPFTFGGGTKVEIK

SEQ ID NO: 435 - protein, 107 aa, Artificial Sequence
  source: /note=“Description of Artificial Sequence Synthetic polypeptide”
DIQMTQSPSSVSASVGDRVTITCRASQGISSWLAWYQQKPGKAPKLLISAASSLQSGVPSRFSGSGSGTDFTLTISSLQPEDFATYYCQQAVILPITFGGGTKVEIK

SEQ ID NO: 436 - protein, 107 aa, Artificial Sequence
  source: /note=“Description of Artificial Sequence Synthetic polypeptide”
DIQMTQSPSTLSASVGDRVTITCRASQSISSWLAWYQQKPGKAPKLLIYKASSLESGVPSRFSGSGSGTEFTLTISSLQPDDFATYYCQQYGILPRTFGGGTKVEIK

SEQ ID NO: 437 - protein, 128 aa, Artificial Sequence
  source: /note=“Description of Artificial Sequence Synthetic polypeptide”
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCARGQYYYGSSSRGYYYMDVWGQGTTVTVSS

SEQ ID NO: 438 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQAVPSPLTFGGGTKVEIK

SEQ ID NO: 439 - protein, 121 aa, Artificial Sequence
  source: /note=“Description of Artificial Sequence Synthetic polypeptide”
EVQLLESGGGLVQPGGSLRLSCAASGFTFSTYAMSWVRQAPGKGLEWVSAISGSGGSTYYADSVKGRFTISRDNSKNTLYLQMNSLRAEDTAVYYCAKGPRYQDRAGMDVWGQGTTVTVSS

SEQ ID NO: 440 - protein, 107 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSRFSGSGSGTDFTLTISSLQPEDFATYYCQQSLATPYTFGGGTKVEIK

SEQ ID NO: 441 - protein, 125 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 442 - protein, 112 aa, Artificial Sequence
  source: /note=“Description of Artificial Sequence Synthetic polypeptide”
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 443 - protein, 121 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGGIIPIFGTASYAQKFQGRVTITADESTSTAYMELSSLRSEDTAVYYCARQSTWHKLYGTDVWGQGTTVTVSS

SEQ ID NO: 444 - protein, 107 aa, Artificial Sequence
  source: /note=“Description of Artificial Sequence Synthetic polypeptide”
DIQMTQSPSSVSASVGDRVTITCRASQGISSWLAWYQQKPGKAPKLLIYAASSLQSGVPSRFSGSGSGTDFTLTISSLQPEDFATYYCQQGDSLPPTFGGGTKVEIK

SEQ ID NO: 445 - protein, 125 aa, Artificial Sequence
  source: /note=“Description of Artificial Sequence Synthetic polypeptide”
QVQLVQSGAEVKKPGSSVKVSCKASGGTFLSSAISWVRQAPGQGLEWMGSLIPYFGTANYAQKFQGRVTITADESTSTAYMELSSLRSEDTAVYYCARGPSEVGAILGYVWFDPWGQGTLVTVSS

SEQ ID NO: 446 - protein, 125 aa, Artificial Sequence
  source: /note=“Description of Artificial Sequence Synthetic polypeptide”
QVQLVQSGAEVKKPGSSVKVSCKASGGTFSAWAISWVRQAPGQGLEWMGSIIPYFGKANYAQKFQGRVTITADESTSTAYMELSSLRSEDTAVYYCARGPSEVSGILGYVWFDPWGQGTLVTVSS

SEQ ID NO: 447 - protein, 125 aa, Artificial Sequence
  source: /note=“Description of Artificial Sequence Synthetic polypeptide”
QVQLVQSGAEVKKPGSSVKVSCKASGGTFLSSAISWVRQAPGQGLEWMGSIIPLFGKANYAQKFQGRVTITADESTSTAYMELSSLRSEDTAVYYCARGPSEVKGILGYVWFDPWGQGTLVTVSS

SEQ ID NO: 448 - protein, 125 aa, Artificial Sequence
  source: /note=“Description of Artificial Sequence Synthetic polypeptide”
QVQLVQSGAEVKKPGSSVKVSCKASGGTFLSSAISWVRQAPGQGLEWMGSIIPYFGKANYAQKFQGRVTITADESTSTAYMELSSLRSEDTAVYYCARGPSEVKGILGYVWFDPWGQGTLVTVSS